SEQUENCE LISTING (303 sequences)

SEQ ID NO: 1 - protein, 4 aa, synthetic construct
GDIR

SEQ ID NO: 2 - protein, 4 aa, synthetic construct
GDIK

SEQ ID NO: 3 - protein, 24 aa, synthetic construct
MPMGSLQPLATLYLLGMLVASVLA

SEQ ID NO: 4 - protein, 4 aa, unidentified
  source 1..4: note = cloning site/kozak sequence
VDTA

SEQ ID NO: 5 - protein, 4 aa, Human immunodeficiency virus
REKR

SEQ ID NO: 6 - protein, 9 aa, Human immunodeficiency virus
RRVVEREKR

SEQ ID NO: 7 - protein, 9 aa, synthetic construct
ERVVEREKE

SEQ ID NO: 8 - protein, 4 aa, synthetic construct
SEKS

SEQ ID NO: 9 - protein, 79 aa, synthetic construct
MRVMGIQRNYPQWWIWSMLGFWMLMICNGMWVTVYYGVPVWKEAKTTLFCASDAKAYEKEVHNVWATHACVPTDPNPQE

SEQ ID NO: 10 - RNA, 101 nt, synthetic construct
  source 1..101: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 11 - RNA, 200 nt, synthetic construct
  source 1..200: mol_type = other RNA
  misc_difference 86..200: note = Nucleotide may be deleted
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 12 - RNA, 110 nt, synthetic construct
  source 1..110: mol_type = other RNA
  misc_difference 86..110: note = Nucleotide may be deleted
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 13 - RNA, 110 nt, synthetic construct; identical to SEQ ID NO: 12
  source 1..110: mol_type = other RNA
  misc_difference 91..110: note = Nucleotide may be deleted

SEQ ID NO: 14 - protein, 5 aa, synthetic construct
  VARIANT 3: note = Any amino acid
LPXTG

SEQ ID NO: 15 - protein, 6 aa, synthetic construct
  VARIANT 3: note = Any amino acid
LPXTGG

SEQ ID NO: 16 - DNA, 145 bp, synthetic construct
  source 1..145: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgct

SEQ ID NO: 17 - DNA, 165 bp, synthetic construct
  source 1..165: mol_type = other DNA
actagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattct

SEQ ID NO: 18 - protein, 5 aa, synthetic construct
GGGGG

SEQ ID NO: 19 - protein, 24 aa, Homo sapiens
MPMGSLQPLATLYLLGMLVASVLA

SEQ ID NO: 20 - protein, 847 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGAKRRVVEREKRAAGLGALFLGFLGAAGSTMGAASITLTVQARQLLSGIVQQQSNLLRAIEAQQHMLQLTVWGIKQLQARVLALERYLKDQQLLGMWGCSGKLICTTNVPWNTSWSNKSETDIWDNMTWMQWEREISNYTETIYKLLEDSQNQQERNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 21 - protein, 853 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASITLTVQARQLLSGIVQQQSNLLRAIEAQQHMLQLTVWGIKQLQARVLALERYLKDQQLLGMWGCSGKLICCTNVPWNTSWSNKSETDIWDNMTWMQWEREISNYTETIYKLLEDSQNQQERNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 22 - protein, 853 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASITLTVQARQLLGSAGSAGSGSAGSGSAGSGSAGSTVWGIKQLQARVLALERYLKDQQLLGMWGCSGKLICCTNVPWNTSWSNKSETDIWDNMTWMQWEREISNYTETIYKLLEDSQNQQERNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 23 - protein, 853 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASITLTVQARQLLSGIVQQQSNLLRAPEAQQHMLQPPVWGIKQLQARVLALERYLKDQQLLGMWGCSGKLICCTNVPWNTSWSNKSETDIWDNMTWMQWEREISNYTETIYKLLEDSQNQQERNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 24 - protein, 853 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNTTTELRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLGSAGSAGSGSAGSGSAGSGSAGSTVWGIKQLQARVLALERYLKDQQLLGMWGCSGKLICCTNVPWNTSWSNKSETDIWDNMTWMQWEREISNYTETIYKLLEDSQNQQEKNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 25 - protein, 853 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNTTTELRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLSGIVQQQSNLLRAPEAQQHMLQPPVWGIKQLQARVLALERYLKDQQLLGMWGCSGKLICCTNVPWNTSWSNKSETDIWDNMTWMQWEREISNYTETIYKLLEDSQNQQEKNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 26 - protein, 853 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNMTTELRDKKQKVYSLFYRLDVVQINENQGNRSNNSNKEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGAKRRVVEREKRAAGLGALFLGFLGAAGSTMGAASITLTVQARQLLSGIVQQQSNLLRAIEAQQHMLQLTVWGIKQLQARVLALERYLKDQQLLGMWGCSGKLICTTNVPWNTSWSNKSETDIWDNMTWMQWEREISNYTETIYKLLEDSQNQQERNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 27 - protein, 859 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNMTTELRDKKQKVYSLFYRLDVVQINENQGNRSNNSNKEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASITLTVQARQLLSGIVQQQSNLLRAIEAQQHMLQLTVWGIKQLQARVLALERYLKDQQLLGMWGCSGKLICCTNVPWNTSWSNKSETDIWDNMTWMQWEREISNYTETIYKLLEDSQNQQERNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 28 - protein, 859 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNMTTELRDKKQKVYSLFYRLDVVQINENQGNRSNNSNKEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASITLTVQARQLLGSAGSAGSGSAGSGSAGSGSAGSTVWGIKQLQARVLALERYLKDQQLLGMWGCSGKLICCTNVPWNTSWSNKSETDIWDNMTWMQWEREISNYTETIYKLLEDSQNQQERNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 29 - protein, 859 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNMTTELRDKKQKVYSLFYRLDVVQINENQGNRSNNSNKEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASITLTVQARQLLSGIVQQQSNLLRAPEAQQHMLQPPVWGIKQLQARVLALERYLKDQQLLGMWGCSGKLICCTNVPWNTSWSNKSETDIWDNMTWMQWEREISNYTETIYKLLEDSQNQQERNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 30 - protein, 859 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNMTTELRDKKQKVYSLFYRLDVVQINENQGNRSNNSNKEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLGSAGSAGSGSAGSGSAGSGSAGSTVWGIKQLQARVLALERYLKDQQLLGMWGCSGKLICCTNVPWNTSWSNKSETDIWDNMTWMQWEREISNYTETIYKLLEDSQNQQEKNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 31-32 - skipped (2 entries)

SEQ ID NO: 33 - protein, 657 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNTTTELRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLGSAGSAGSGSAGSGSAGSGSAGSTVWGIKQLQARVLALERYLKDQQLLGMWGCSGKLICCTNVPWNTSWSNKSETDIWDNMTWMNWSREISNYTETIYKLLEDSQNQQEKNNQSLLALDNGS

SEQ ID NO: 34 - skipped

SEQ ID NO: 35 - protein, 656 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICTNYAPKLRSMMRGEIKNCSFNMTTELRDKKQKVYSLFYRLDVVQINENQGNRSNNSNKEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 36 - protein, 650 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICTNYAPKLRSMMRGEIKNCSFNTTTEIRDKEKKEYALFYKPDIVPLSETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 37 - protein, 650 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICTNYAPKLRSMMRGEIKNCSFNTTTEIRDKEKKEYALFYKPDIVPLSETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKPPVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 38 - protein, 650 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICTNYAPKLRSMMRGEIKNCSFNTTTEIRDKKKKEYALFYKPDIVPLSETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKPPVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 39 - protein, 648 aa, synthetic construct
MGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNTTTEIRDKKKKEYALFYKPDIVPLSETNNTSEYRLINCNTSAVTQVCPKLTFEPIPIHYCAPAGYAILKCNDETFNGTGPCSNVSTVQCTHGIRPVLSTQLLLNGSLAEKEIVIRSENLTNNAKIIIVHLHTPVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 40 - protein, 648 aa, synthetic construct
MGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNTTTEIRDKKKKEYALFYKPDIVPLSETNNTSEYRLINCNTSACTQVCPKLTFEPIPIHYCAPAGYAILKCNDETFNGTGPCSNVSTVQCTHGIRPVLSTQLLLNGSLAEKEIVIRSENLTNNAKIIIVHLHTPVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 41 - protein, 720 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICTNYAPKLRSMMRGEIKNCSFNTTTEIRDKKKKEYALFYKPDIVPLSETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKPPVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGSEQKLISEEDLLEVLFQGPGAVGQDTQEVIVVPHSLPFKVVVISAILALVVLTIISLIILIMLWQKKPR

SEQ ID NO: 42 - protein, 720 aa, synthetic construct; identical to SEQ ID NO: 41

SEQ ID NO: 43 - protein, 663 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNMTTELRDKKQKVYSLFYRLDVVQINENQGNRSNNSNKEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLGSAGSAGSGSAGSGSAGSGSAGSTVWGIKQLQARVLALERYLKDQQLLGMWGCSGKLICCTNVPWNTSWSNKSETDIWDNMTWMNWSREISNYTETIYKLLEDSQNQQEKNNQSLLALDNGS

SEQ ID NO: 44 - protein, 663 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNMTTELRDKKQKVYSLFYRLDVVQINENQGNRSNNSNKEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLSGIVQQQSNLLRAPEAQQHMLQPPVWGIKQLQARVLALERYLKDQQLLGMWGCSGKLICCTNVPWNTSWSNKSETDIWDNMTWMNWSREISNYTETIYKLLEDSQNQQEKNNQSLLALDNGS

SEQ ID NO: 45 - protein, 657 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNTTTELRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLSGIVQQQSNLLRAPEAQQHMLQPPVWGIKQLQARVLALERYLKDQQLLGMWGCSGKLICCTNVPWNTSWSNKSETDIWDNMTWMNWSREISNYTETIYKLLEDSQNQQEKNNQSLLALDNGS

SEQ ID NO: 46 - DNA, 1995 bp, synthetic construct
  source 1..1995: mol_type = other DNA
atgcctatgggcagcctgcaacctctggccacactgtatctcctgggcatgctggtcgcctctgtgctggccaagggcaagctgtgggtgacagtgtactacggcgtgcctgtgtggaaagaggctaaaaccacactgttttgcgccagcgacgcccgcgcctatgagaaggaggtgcacaacatctgggccacccacgcctgtgtgccaacagacccttctccacaggagctggtgctgggcaacgtgaccgagaacttcaacatgtggaagaacgacatggtggaccagatgcacgaggacatcatcagcctgtgggaccagagcctgaagccctgcgtgaaactgacacctctgtgcgtgaccctgatttgtacaaactacgcccctaagctgagaagcatgatgagaggcgagatcaagaactgctccttcaacatgaccaccgagcttagagataagaagcaaaaggtctacagcctcttctacagactggatgtggtgcagatcaacgagaatcaaggcaatagaagcaacaacagcaacaaagaataccggttgatcaattgcaacacatctgccgtgacccaggtgtgccccaagctgaccttcgagcctatccctatccactactgcgccccagccggctacgctatcctgaagtgcaacaatgagacattcaatggcactggcccttgtagcaatgtgtccaccgtgcagtgcacgcacggcatcagacctgtgctgagcacacagctgctgctgaacggcagcctggccgagaaagagatcgtgatcagaagcgagaacctgacaaacaacgccaagatcatcatcgtgcacctgaacacctctgttgaaatcgtctgcacaagacctaacaacaacaccagaaagagcgtgcggatcggacctggacagaccttctacgccaccggcgacattatcggcgatatcaagcaggctcactgtaatatcagcgaggaaaagtggaatgatacactgcagaaggtgggcatcgagctgcagaagcacttccccaacaagaccatcaagtacaaccagtctgctggaggtgacatggaaattacaacacattcttttaactgcggcggagagttcttctactgtaacaccagcaatctgttcaacggcacatataacgggacctacattagcaccaattctagcgccaacagcaccagcaccattaccctgcaatgcaggatcaagcagatcatcaacatgtggcagggcgtgggaagagccatgtacgccccccccatcgccggcaacatcacctgcagatccaatatcacaggtctgctactgacccgggacggcggaacaaatagcaatgagacagaaacctttcggcccgccggcggtgacatgcgggataactggcggagcgaactgtacaagtacaaagtggtcgaaatccagcctctgggcatcgcccctaccggatgtaaacggagagtggtggaaggcggcggaggcagcggcggcggcggcagcgcagccggcctgggagccctgttcctgggcttcctgggcgctgctggcagcaccatgggagccgccagcatgactctcaccgttcaagccagacagctactgggcagcgctggctccgccggaagtggctccgctggctcgggcagcgccggaagcggcagcgccggcagcaccgtgtggggaatcaagcagctgcaggctagagtgctcgccctggaacggtacctgaaggaccagcagctgctcggcatgtggggctgttccggcaagctgatctgctgcaccaacgtgccctggaacaccagttggtccaacaagtctgaaaccgacatctgggataacatgacctggatgaactggagcagagaaatcagtaactacaccgaaaccatctacaagctgctggaagatagccagaaccagcaggagaaaaacaaccagagcctgctggccctggacaacggctcctgatga

SEQ ID NO: 47 - DNA, 1995 bp, synthetic construct
  source 1..1995: mol_type = other DNA
atgcctatgggctctctgcagcccctggctaccctgtacctgctgggcatgctggtcgccagcgttctggccaagggcaagctgtgggtgaccgtgtactacggcgttcctgtgtggaaggaagccaagaccacccttttttgtgcctctgacgcccgcgcatatgagaaggaagtgcacaacatctgggccacacacgcctgtgtgcccacagaccctagcccccaagagctggtgctgggaaatgtgacagagaacttcaacatgtggaagaacgacatggtggaccagatgcatgaagatatcatctccctgtgggaccagagcctgaaaccttgcgtgaagctgacacccctgtgtgtgacccttatttgcaccaactacgcccctaagctgaggtccatgatgcggggcgagatcaagaactgcagcttcaatatgacaaccgagctgagagataagaagcagaaagtgtacagcctgttctatagactggacgtggtacagatcaacgagaaccaaggcaacagaagcaataacagcaataaggagtaccggctgatcaattgcaacaccagcgccgtgacccaggtgtgccccaagctgaccttcgagcctatccctatccactactgcgcccccgccggatacgccatcctgaagtgcaacaatgagacattcaatggcaccggcccctgcagcaacgtgtccacagtgcagtgcacccacggcattagaccagtgctgtctactcagctgctgctgaacggctcactggcagaaaaggaaatcgtgatccggagcgagaatctgaccaataacgccaagatcattatcgttcacctgaatacatctgtggaaatcgtgtgtacaagacctaacaacaacacccgaaagagcgtgcggatcggacctggccagacattctacgccaccggcgatatcatcggcgacatcaagcaagctcactgcaacatcagcgaggaaaagtggaacgacaccctgcagaaggtgggcatcgagctgcagaagcacttcccaaacaagacaatcaaatacaaccagtctgccggaggcgatatggaaatcaccacccattcttttaattgtggcggcgaattcttctactgcaacaccagcaacctgtttaacggaacctacaacggcacctacatcagcaccaacagcagcgctaatagcacaagtaccatcaccctgcagtgtcggatcaagcagataatcaacatgtggcagggcgtcggcagagctatgtacgccccacctatcgctggcaacattacatgcagaagcaatatcaccggcctgctgctgacccgggacggcggtacaaacagcaacgagacagaaacctttagaccagccggcggcgacatgagagataactggagaagcgagctgtacaagtacaaagtcgtggaaatccagcctctgggcatcgcccccaccggctgcaagcggagagtggtggaaggcggaggaggctctggcggtggaggctcggctgccggcctgggcgccctgttcctgggcttcctgggagccgctggcagcacaatgggagccgcctccatgaccctgacagtccaggcccggcagctgctcagcggaatcgtgcagcagcaatctaacctgcttagagcccctgaagcccagcaacacatgctgcaacctcctgtgtggggcatcaagcagctgcaggccagagtgctggctctggaaagatacctgaaagaccagcagctgttaggcatgtggggatgtagcggcaagttgatctgctgcaccaacgtgccttggaataccagctggagtaacaaaagcgagacagatatctgggataacatgacctggatgaactggagcagagagatcagcaactacaccgagaccatctacaagctgctcgaggacagccagaaccagcaggaaaagaacaaccagtccctgctggccctggacaacggaagctgatga

SEQ ID NO: 48 - DNA, 1977 bp, synthetic construct
  source 1..1977: mol_type = other DNA
atgcctatgggaagcctgcagcccctggccacactgtacctgctgggcatgctggtggccagcgtgctggctaaaggcaagctgtgggtcaccgtgtactacggcgtgcccgtgtggaaggaggccaagaccacactgttctgcgcctctgatgccagagcctacgagaaagaggtgcacaacatttgggccacacacgcctgcgtgcctacagaccccagcccccaggagctggtgctggggaacgtgaccgagaatttcaacatgtggaagaacgacatggtggaccagatgcacgaggacatcatcagcctgtgggatcagagcctgaagccctgcgtgaagctaacacctctgtgcgtcaccctgatctgtacaaactacgctccaaagctgcggagcatgatgcggggcgaaatcaaaaactgcagctttaataccaccaccgaactgagagataagaaaaagaaggaatacgccctgttctacaagcctgacatcgtgcctctgagcgagacaaataacacaagcgagtacagactgatcaactgcaatactagcgctgtgacccaggtttgtcctaagctgaccttcgagcctatccccatccactactgcgccccagccggctacgccatcctgaaatgcaacaatgagacattcaacggcaccggaccttgttccaacgtgtccaccgttcagtgtacccacggcatcagacctgtgctgagcacccagctgttgcttaatggatctctggccgagaaggaaatcgtgatccggtccgaaaacctgaccaacaacgccaagatcatcatcgtccatctgaacaccagcgtggaaatcgtgtgcacaaggcctaacaacaacacccggaagtccgtgcggatcggccctggccagaccttctatgctacaggcgacatcattggcgacatcaagcaggcccactgcaacatcagcgaggaaaaatggaacgacaccctgcaaaaggtgggcatcgagctgcaaaagcacttccccaacaaaaccatcaagtacaaccagagcgccggcggagatatggaaattacaacccacagcttcaattgcggcggcgagttcttctattgtaataccagcaacctgtttaacggcacctacaacggcacatacatcagcacaaattctagcgccaactccacgagcaccatcactctgcagtgcagaatcaaacagatcatcaacatgtggcagggcgtgggtagagccatgtacgccccacctatcgccggaaacatcacgtgtagaagcaacatcaccggcctgctgcttacacgggacggcggcaccaacagcaacgagaccgagaccttcagacccgccggaggcgacatgagagataactggcggtctgagctgtataagtacaaggtggtggaaattcagcctctgggcatcgcccctaccggctgtaaaagaagagtggtggaaggcggcggaggctctggcggcggcggctctgctgccggactgggcgctctgtttctggggttcctgggagccgctggctccacaatgggcgccgcgagcatgaccctgacagtgcaggcccgccagctgctgggctctgctggaagcgctggaagcggatctgccggctccggttctgccggcagcggcagcgccggcagcaccgtgtggggcatcaagcagctgcaagccagagtgctggccctggaaagatacctgaaggaccaacagctgctgggcatgtggggctgcagcggcaagctgatctgctgcaccaacgtaccttggaataccagctggtccaacaagagcgagacagatatctgggacaatatgacctggatgaactggagcagagagatcagcaattacaccgagaccatctacaagctgctggaagattcccagaaccagcaggagaagaacaaccagagcctgttggctctggacaacggctcttgatga

SEQ ID NO: 49 - DNA, 1977 bp, synthetic construct
  source 1..1977: mol_type = other DNA
atgcctatgggcagcctgcagcctctggccacactgtacctgctcggcatgctggtcgccagcgttttggccaagggcaagctgtgggtgaccgtgtactacggcgtgcccgtgtggaaggaagccaaaacaacactgttctgcgccagtgatgccagagcctacgaaaaagaagtgcataatatctgggccacccacgcctgcgtgcccaccgatcctagccctcaggagctggtgctgggcaacgtgacagagaacttcaacatgtggaagaacgacatggtggaccagatgcacgaggacatcatctctctgtgggaccagtctctgaagccctgtgtgaagctgacccctctgtgcgtgacactcatctgcaccaattacgcccctaagctgagaagcatgatgagaggcgagatcaagaactgttctttcaacaccacaaccgagctgcgcgataagaaaaagaaagagtatgccctgttctacaagcccgacatcgtgcctctgagcgaaacaaacaacacctctgaatataggctgatcaactgcaacaccagcgccgtgacccaggtgtgccccaagctgacattcgagcctatccccatccactactgtgcccctgctggctacgccatcctgaagtgcaacaacgagacgttcaacggcaccggcccttgcagcaacgtgtccacagtgcagtgtacccacggcatccggcctgtgctcagcacccagctgctgctgaacggcagcctggccgagaaggagatcgtgatcagaagcgaaaatctgacaaacaacgccaagatcattatcgtgcacctgaacacaagcgtcgagattgtctgcaccagaccgaataacaacaccagaaagtccgtgcggatcggacctggccagaccttctacgctaccggcgacatcatcggcgatatcaagcaggcccactgcaatatctccgaggaaaagtggaatgacaccctgcagaaggttggaatcgagctgcaaaagcacttccccaacaagaccatcaaatacaaccagagcgccggcggagatatggaaatcaccacccactcatttaattgcggcggggagttcttctattgtaacacatctaacctgttcaatggcacctacaacggcacctacatctccaccaacagcagcgctaatagcactagcaccattacactgcaatgtagaatcaagcagatcattaacatgtggcagggcgtgggcagagccatgtacgctccaccaatcgccggaaatatcacctgcagaagcaacatcacaggtcttctgctgacccgggacggcggaaccaacagcaacgagacagagacctttagacccgccggaggcgacatgcgggacaactggcggagcgagctgtacaagtacaaggtggtcgagatccagcctctcggcatcgcccctacaggatgtaaaagaagggtggtggagggcggcggtggctccggcggcggcggatctgctgccggcctgggagccctgtttctgggcttcctgggcgccgccggctctacaatgggagctgctagcatgacactgaccgtgcaggcccggcagctgctgtctggcatcgtgcagcagcagagcaacctgctgcgggcccctgaggcccagcaacacatgctgcagcctccagtgtggggaataaagcagctgcaggctagagtgctggcactggaaagatacctgaaagaccagcaactgctgggcatgtggggctgcagcggcaagctgatttgctgcaccaacgtgccatggaacaccagctggtccaacaagagcgaaaccgacatctgggacaatatgacctggatgaactggagcagagagatcagcaactacaccgaaaccatctacaaactgctggaagatagccagaaccagcaggagaagaacaaccaaagcctgctggccctcgacaatggctcttgatga

SEQ ID NO: 50 - DNA, 1974 bp, synthetic construct
  source 1..1974: mol_type = other DNA
atgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtacaaactacgcccctaagctgagaagcatgatgagaggcgagatcaagaactgctccttcaacatgaccaccgagcttagagataagaagcaaaaggtctacagcctcttctacagactggatgtggtgcagatcaacgagaatcaaggcaatagaagcaacaacagcaacaaagaataccggttgatcaattgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggattgatga

SEQ ID NO: 51 - DNA, 2166 bp, synthetic construct
  source 1..2166: mol_type = other DNA
atgccaatgggctcccttcagcccttggctaccctgtaccttcttgggatgttggtcgcatctgtgcttgccgcggagaacctgtgggtgacggtctattatggagtaccggtctggaaagaggcaaaaaccaccctcttttgcgccagcgacgctagggcctacgagaaagaagtgcataatgtgtgggcgactcacgcttgcgtcccgaccgatcccagtccccaggagctcgtattgggtaacgtaacagagaacttcaatatgtggaagaatgacatggtggaccagatgcatgaagatatcatatccctttgggaccagagtcttaaaccctgcgttaaattgaccccgctttgcgtgacgcttatctgtaccaactacgcccccaagctgaggtccatgatgaggggcgagatcaagaattgctcatttaatacaacgacggaaatcagggataagaagaaaaaagaatacgcattgttttataagccagatatcgttcctctgtcagagactaataataccagtgagtacaggcttattaactgtaatacatccgcatgtacacaggcgtgccctaaagttacatttgagccgattccgattcattattgtgcacctgcggggtatgctattcttaaatgcaacgatgagaccttcaatggcaccggtccgtgttccaatgtatcaaccgtccagtgtacccacggtatccgaccagtggttagtactcagctcttgctcaatggctcactcgcggaaaaagaaattgtaatcagatctgaaaatcttacaaataacgccaaaatcattattgtccatctccatacccctgtagagatcgtatgtactcgaccaaacaataatacacgcaagagtgtaaggattgggccgggccagacattctatgcgactggagacattatcggcgatataaaacaagcgcattgtaacatcagtgaagagaagtggaacgatactttgcagaaagtgggaatcgaactgcaaaagcactttccaaacaaaactataaaatataaccagagtgcagggggagatatggagatcacaacccacagctttaattgcggcggcgagtttttctattgcaacacttccaacctctttaatggaacttataacgggacttatatctcaactaattctagtgctaattcaacgagcacgataacacttcaatgcagaatcaaacagatcatcaacatgtggcagggcgttggtcgctgcatgtacgcccctcctatagcaggtaatattacgtgcaggtctaatatcacaggtttgctcctgacgcgcgacgggggcaccaactcaaacgagacggagacgttccggcctgctggcggagatatgagagataactggagatctgaactgtataaatacaaggtagtgaaaatagaacctttgggagtggcacccaccagatgcaagcggcgagtggttggacgccgccgcagacgacgagccgttggaattggagcagtatttcttggctttctcggggcggcaggctccactatgggcgcagcatccatgaccttgaccgttcaggccaggaatcttttgtccggcatcgtccagcaacagagcaacctgcttcgggctccagaagcacagcagcatcttctcaagcccccagtttggggaatcaagcaacttcaggcaagagttttggccgtagagcgatatctcagggaccagcaactcctcggtatatggggctgtagtggaaagcttatttgttgcaccaacgttccatggaatagttcctggtccaatcgcaatctttccgagatatgggacaatatgacctggttgcagtgggacaaagaaatttcaaactatacccaaattatttatgggttgcttgaggagtcacagaaccagcaagaaaagaacgaacaggaccttctggcccttgatggctctgaacagaaactgatctctgaggaggacctgcttgaggtgctgtttcagggacctggcgccgtgggccaggacacacaggaggtgatcgtggtgccccatagcctgcctttcaaggtggttgtgatcagcgccatcctggcactggtggtgctgacaatcatcagcctgataatcctgatcatgctgtggcaaaagaaacccagataatga

SEQ ID NO: 52 - protein, 853 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNTTTELRDKKKKEYALFYKPDIVPLSETNNTSEYRLINCNTSACTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQWFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLGSAGSAGSGSAGSGSAGSGSAGSTVWGIKQLQARVLALERYLKDQQLLGMWGCSGKLICCTNVPWNTSWSNKSETDIWDNMTWMQWEREISNYTETIYKLLEDSQNQQEKNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 53 - protein, 853 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNTTTELRDKKKKEYALFYKPDIVPLSETNNTSEYRLINCNTSACTMVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPLNLTRKSVRIGPGQWFYAMGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKMIINMWQGVGRCMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLGSAGSAGSGSAGSGSAGSGSAGSTVWGIKQLQARVLALERYLKDQQLLGMWGCSGKLICCTNVPWNTSWSNKSETDIWDNMTWMQWEREISNYTETIYKLLEDSQNQQEKNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 54 - protein, 853 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNTTTELRDKKKKEYALFYKPDIVPLSETNNTSEYRLINCNTSAVTMVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPLNLTRKSVRIGPGQTFYAMGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKMIINMWQGVGRAMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLGSAGSAGSGSAGSGSAGSGSAGSTVWGIKQLQARVLALERYLKDQQLLGMWGCSGKLICCTNVPWNTSWSNKSETDIWDNMTWMQWEREISNYTETIYKLLEDSQNQQEKNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 55 - protein, 853 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNTTTELRDKKKKEYALFYKPDIVPLSETNNTSEYRLINCNTSACTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQWFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLGSAGSAGSGSAGSGSAGSGSAGSTVWGIKQLQARVLALERYLKDQQLLGMWGCSGKLICCTNVPWNTSWSNKSETDIWDNMTWMQWEREISNYTETIYKLLEDSQNQQEKNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASAA

SEQ ID NO: 56 - protein, 853 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNTTTELRDKKKKEYALFYKPDIVPLSETNNTSEYRLINCNTSACTMVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPLNLTRKSVRIGPGQWFYAMGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKMIINMWQGVGRCMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLGSAGSAGSGSAGSGSAGSGSAGSTVWGIKQLQARVLALERYLKDQQLLGMWGCSGKLICCTNVPWNTSWSNKSETDIWDNMTWMQWEREISNYTETIYKLLEDSQNQQEKNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASAA

SEQ ID NO: 57 - protein, 853 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNTTTELRDKKKKEYALFYKPDIVPLSETNNTSEYRLINCNTSAVTMVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPLNLTRKSVRIGPGQTFYAMGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKMIINMWQGVGRAMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLGSAGSAGSGSAGSGSAGSGSAGSTVWGIKQLQARVLALERYLKDQQLLGMWGCSGKLICCTNVPWNTSWSNKSETDIWDNMTWMQWEREISNYTETIYKLLEDSQNQQEKNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASAA

SEQ ID NO: 58 - protein, 745 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNTTTELRDKKKKEYALFYKPDIVPLSETNNTSEYRLINCNTSACTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQWFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLGSAGSAGSGSAGSGSAGSGSAGSTVWGIKQLQARVLALERYLKDQQLLGMWGCSGKLICCTNVPWNTSWSNKSETDIWDNMTWMQWEREISNYTETIYKLLEDSQNQQEKNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPI

SEQ ID NO: 59 - protein, 745 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNTTTELRDKKKKEYALFYKPDIVPLSETNNTSEYRLINCNTSACTMVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPLNLTRKSVRIGPGQWFYAMGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKMIINMWQGVGRCMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLGSAGSAGSGSAGSGSAGSGSAGSTVWGIKQLQARVLALERYLKDQQLLGMWGCSGKLICCTNVPWNTSWSNKSETDIWDNMTWMQWEREISNYTETIYKLLEDSQNQQEKNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPI

SEQ ID NO: 60 - protein, 745 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNTTTELRDKKKKEYALFYKPDIVPLSETNNTSEYRLINCNTSAVTMVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPLNLTRKSVRIGPGQTFYAMGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKMIINMWQGVGRAMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLGSAGSAGSGSAGSGSAGSGSAGSTVWGIKQLQARVLALERYLKDQQLLGMWGCSGKLICCTNVPWNTSWSNKSETDIWDNMTWMQWEREISNYTETIYKLLEDSQNQQEKNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPI

SEQ ID NO: 61 - protein, 702 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNTTTELRDKKKKEYALFYKPDIVPLSETNNTSEYRLINCNTSACTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQWFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLGSAGSAGSGSAGSGSAGSGSAGSTVWGIKQLQARVLALERYLKDQQLLGMWGCSGKLICCTNVPWNTSWSNKSETDIWDNMTWMQWEREISNYTETIYKLLEDSQNQQEKNEQDLLALDSWNSLWNWFSITKWLWYIKIFIMIVGGLIGLRIVFAVLSIVNRVRQGI

SEQ ID NO: 62 - protein, 702 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNTTTELRDKKKKEYALFYKPDIVPLSETNNTSEYRLINCNTSACTMVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPLNLTRKSVRIGPGQWFYAMGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKMIINMWQGVGRCMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLGSAGSAGSGSAGSGSAGSGSAGSTVWGIKQLQARVLALERYLKDQQLLGMWGCSGKLICCTNVPWNTSWSNKSETDIWDNMTWMQWEREISNYTETIYKLLEDSQNQQEKNEQDLLALDSWNSLWNWFSITKWLWYIKIFIMIVGGLIGLRIVFAVLSIVNRVRQGI

SEQ ID NO: 63 - protein, 702 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRSMMRGEIKNCSFNTTTELRDKKKKEYALFYKPDIVPLSETNNTSEYRLINCNTSAVTMVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPLNLTRKSVRIGPGQTFYAMGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKMIINMWQGVGRAMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLGSAGSAGSGSAGSGSAGSGSAGSTVWGIKQLQARVLALERYLKDQQLLGMWGCSGKLICCTNVPWNTSWSNKSETDIWDNMTWMQWEREISNYTETIYKLLEDSQNQQEKNEQDLLALDSWNSLWNWFSITKWLWYIKIFIMIVGGLIGLRIVFAVLSIVNRVRQGI

SEQ ID NO: 64 - DNA, 2964 bp, synthetic construct
  source 1..2964: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacaccaccaccgagatccgcgacaagaagaagaaggagtacgccctgttctacaagcccgacatcgtgcccctgtccgagaccaacaacacctccgagtaccgcctgatcaactgcaacacctccgccgtgacccaggtgtgccccaagctgaccttcgagcccatccccatccactactgcgcccccgccggctacgccatcctgaagtgcaacaacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgaacacctccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgcgccatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtggagatccagcccctgggcatcgcccccaccggcgccaagcgccgcgtggtggagcgcgagaagcgcgccgccggcctgggcgccctgttcctgggcttcctgggcgccgccggctccaccatgggcgccgcctccatcaccctgaccgtgcaggcccgccagctgctgtccggcatcgtgcagcagcagtccaacctgctgcgcgccatcgaggcccagcagcacatgctgcagctgaccgtgtggggcatcaagcagctgcaggcccgcgtgctggccctggagcggtacctgaaggaccagcagctgctgggcatgtggggctgctccggcaagctgatctgcaccaccaacgtgccctggaacacctcctggtccaacaagtccgagaccgacatctgggacaacatgacctggatgcagtgggagcgcgagatctccaactacaccgagaccatctacaagctgctggaggactcccagaaccagcaggagcgcaacgagcaggacctgctggccctggactcctggaactccctgtggaactggttctccatcaccaagtggctgtggtacatcaagatcttcatcatgatcgtgggcggcctgatcggcctgcgcatcgtgttcgccgtgctgtccatcgtgaaccgcgtgcgccagggcatctcccccctgtccctgcagaccctgacccccaacccccgcgagcccgaccgcctgcgcggcatcgaggaggagggcggcgagcaggaccgcgaccgctccatccgcctggtgtccggcttcctgcccatcgtgtgggacgacctgcgctccctgtgcctgttctcctaccaccgcctgcgcgacttcctgctgctggccgcccgcgtggtggagctgctgggccgctcctccctgcgcggcctgcagcgcggctgggaggtgctgaagtacctgggctccctggtgcagtactggggcctggagctgaagaagtccgccatctccctgttcgacaccctggccatcgccgtggccgagggcaccgaccgcatcatcgagctgatccagggcttctgccgcgccatccgcaacatccccacccgcatccgccagggcttcgaggcctccctgctgtaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 65 - DNA, 2982 bp, synthetic construct
  source 1..2982: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacaccaccaccgagatccgcgacaagaagaagaaggagtacgccctgttctacaagcccgacatcgtgcccctgtccgagaccaacaacacctccgagtaccgcctgatcaactgcaacacctccgccgtgacccaggtgtgccccaagctgaccttcgagcccatccccatccactactgcgcccccgccggctacgccatcctgaagtgcaacaacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgaacacctccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgcgccatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtggagatccagcccctgggcatcgcccccaccggctgcaagcgccgcgtggtggagggcggcggcggctccggcggcggcggctccgccgccggcctgggcgccctgttcctgggcttcctgggcgccgccggctccaccatgggcgccgcctccatcaccctgaccgtgcaggcccgccagctgctgtccggcatcgtgcagcagcagtccaacctgctgcgcgccatcgaggcccagcagcacatgctgcagctgaccgtgtggggcatcaagcagctgcaggcccgcgtgctggccctggagcggtacctgaaggaccagcagctgctgggcatgtggggctgctccggcaagctgatctgctgcaccaacgtgccctggaacacctcctggtccaacaagtccgagaccgacatctgggacaacatgacctggatgcagtgggagcgcgagatctccaactacaccgagaccatctacaagctgctggaggactcccagaaccagcaggagcgcaacgagcaggacctgctggccctggactcctggaactccctgtggaactggttctccatcaccaagtggctgtggtacatcaagatcttcatcatgatcgtgggcggcctgatcggcctgcgcatcgtgttcgccgtgctgtccatcgtgaaccgcgtgcgccagggcatctcccccctgtccctgcagaccctgacccccaacccccgcgagcccgaccgcctgcgcggcatcgaggaggagggcggcgagcaggaccgcgaccgctccatccgcctggtgtccggcttcctgcccatcgtgtgggacgacctgcgctccctgtgcctgttctcctaccaccgcctgcgcgacttcctgctgctggccgcccgcgtggtggagctgctgggccgctcctccctgcgcggcctgcagcgcggctgggaggtgctgaagtacctgggctccctggtgcagtactggggcctggagctgaagaagtccgccatctccctgttcgacaccctggccatcgccgtggccgagggcaccgaccgcatcatcgagctgatccagggcttctgccgcgccatccgcaacatccccacccgcatccgccagggcttcgaggcctccctgctgtaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 66 - DNA, 2982 bp, synthetic construct
  source 1..2982: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacaccaccaccgagatccgcgacaagaagaagaaggagtacgccctgttctacaagcccgacatcgtgcccctgtccgagaccaacaacacctccgagtaccgcctgatcaactgcaacacctccgccgtgacccaggtgtgccccaagctgaccttcgagcccatccccatccactactgcgcccccgccggctacgccatcctgaagtgcaacaacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgaacacctccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgcgccatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtggagatccagcccctgggcatcgcccccaccggctgcaagcgccgcgtggtggagggcggcggcggctccggcggcggcggctccgccgccggcctgggcgccctgttcctgggcttcctgggcgccgccggctccaccatgggcgccgcctccatcaccctgaccgtgcaggcccgccagctgctgggctccgccggctccgccggctccggctccgccggctccggctccgccggctccggctccgccggctccaccgtgtggggcatcaagcagctgcaggcccgcgtgctggccctggagcggtacctgaaggaccagcagctgctgggcatgtggggctgctccggcaagctgatctgctgcaccaacgtgccctggaacacctcctggtccaacaagtccgagaccgacatctgggacaacatgacctggatgcagtgggagcgcgagatctccaactacaccgagaccatctacaagctgctggaggactcccagaaccagcaggagcgcaacgagcaggacctgctggccctggactcctggaactccctgtggaactggttctccatcaccaagtggctgtggtacatcaagatcttcatcatgatcgtgggcggcctgatcggcctgcgcatcgtgttcgccgtgctgtccatcgtgaaccgcgtgcgccagggcatctcccccctgtccctgcagaccctgacccccaacccccgcgagcccgaccgcctgcgcggcatcgaggaggagggcggcgagcaggaccgcgaccgctccatccgcctggtgtccggcttcctgcccatcgtgtgggacgacctgcgctccctgtgcctgttctcctaccaccgcctgcgcgacttcctgctgctggccgcccgcgtggtggagctgctgggccgctcctccctgcgcggcctgcagcgcggctgggaggtgctgaagtacctgggctccctggtgcagtactggggcctggagctgaagaagtccgccatctccctgttcgacaccctggccatcgccgtggccgagggcaccgaccgcatcatcgagctgatccagggcttctgccgcgccatccgcaacatccccacccgcatccgccagggcttcgaggcctccctgctgtaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 67 - DNA, 2982 bp, synthetic construct
  source 1..2982: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacaccaccaccgagatccgcgacaagaagaagaaggagtacgccctgttctacaagcccgacatcgtgcccctgtccgagaccaacaacacctccgagtaccgcctgatcaactgcaacacctccgccgtgacccaggtgtgccccaagctgaccttcgagcccatccccatccactactgcgcccccgccggctacgccatcctgaagtgcaacaacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgaacacctccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgcgccatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtggagatccagcccctgggcatcgcccccaccggctgcaagcgccgcgtggtggagggcggcggcggctccggcggcggcggctccgccgccggcctgggcgccctgttcctgggcttcctgggcgccgccggctccaccatgggcgccgcctccatcaccctgaccgtgcaggcccgccagctgctgtccggcatcgtgcagcagcagtccaacctgctgcgcgcccccgaggcccagcagcacatgctgcagccccccgtgtggggcatcaagcagctgcaggcccgcgtgctggccctggagcggtacctgaaggaccagcagctgctgggcatgtggggctgctccggcaagctgatctgctgcaccaacgtgccctggaacacctcctggtccaacaagtccgagaccgacatctgggacaacatgacctggatgcagtgggagcgcgagatctccaactacaccgagaccatctacaagctgctggaggactcccagaaccagcaggagcgcaacgagcaggacctgctggccctggactcctggaactccctgtggaactggttctccatcaccaagtggctgtggtacatcaagatcttcatcatgatcgtgggcggcctgatcggcctgcgcatcgtgttcgccgtgctgtccatcgtgaaccgcgtgcgccagggcatctcccccctgtccctgcagaccctgacccccaacccccgcgagcccgaccgcctgcgcggcatcgaggaggagggcggcgagcaggaccgcgaccgctccatccgcctggtgtccggcttcctgcccatcgtgtgggacgacctgcgctccctgtgcctgttctcctaccaccgcctgcgcgacttcctgctgctggccgcccgcgtggtggagctgctgggccgctcctccctgcgcggcctgcagcgcggctgggaggtgctgaagtacctgggctccctggtgcagtactggggcctggagctgaagaagtccgccatctccctgttcgacaccctggccatcgccgtggccgagggcaccgaccgcatcatcgagctgatccagggcttctgccgcgccatccgcaacatccccacccgcatccgccagggcttcgaggcctccctgctgtaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 68 - DNA, 2982 bp, synthetic construct
  source 1..2982: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacaccaccaccgagctgcgcgacaagaagaagaaggagtacgccctgttctacaagcccgacatcgtgcccctgtccgagaccaacaacacctccgagtaccgcctgatcaactgcaacacctccgccgtgacccaggtgtgccccaagctgaccttcgagcccatccccatccactactgcgcccccgccggctacgccatcctgaagtgcaacaacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgaacacctccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgcgccatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtggagatccagcccctgggcatcgcccccaccggctgcaagcgccgcgtggtggagggcggcggcggctccggcggcggcggctccgccgccggcctgggcgccctgttcctgggcttcctgggcgccgccggctccaccatgggcgccgcctccatgaccctgaccgtgcaggcccgccagctgctgggctccgccggctccgccggctccggctccgccggctccggctccgccggctccggctccgccggctccaccgtgtggggcatcaagcagctgcaggcccgcgtgctggccctggagcggtacctgaaggaccagcagctgctgggcatgtggggctgctccggcaagctgatctgctgcaccaacgtgccctggaacacctcctggtccaacaagtccgagaccgacatctgggacaacatgacctggatgcagtgggagcgcgagatctccaactacaccgagaccatctacaagctgctggaggactcccagaaccagcaggagaagaacgagcaggacctgctggccctggactcctggaactccctgtggaactggttctccatcaccaagtggctgtggtacatcaagatcttcatcatgatcgtgggcggcctgatcggcctgcgcatcgtgttcgccgtgctgtccatcgtgaaccgcgtgcgccagggcatctcccccctgtccctgcagaccctgacccccaacccccgcgagcccgaccgcctgcgcggcatcgaggaggagggcggcgagcaggaccgcgaccgctccatccgcctggtgtccggcttcctgcccatcgtgtgggacgacctgcgctccctgtgcctgttctcctaccaccgcctgcgcgacttcctgctgctggccgcccgcgtggtggagctgctgggccgctcctccctgcgcggcctgcagcgcggctgggaggtgctgaagtacctgggctccctggtgcagtactggggcctggagctgaagaagtccgccatctccctgttcgacaccctggccatcgccgtggccgagggcaccgaccgcatcatcgagctgatccagggcttctgccgcgccatccgcaacatccccacccgcatccgccagggcttcgaggcctccctgctgtaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 69 - DNA, 2982 bp, synthetic construct
  source 1..2982: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacaccaccaccgagctgcgcgacaagaagaagaaggagtacgccctgttctacaagcccgacatcgtgcccctgtccgagaccaacaacacctccgagtaccgcctgatcaactgcaacacctccgccgtgacccaggtgtgccccaagctgaccttcgagcccatccccatccactactgcgcccccgccggctacgccatcctgaagtgcaacaacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgaacacctccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgcgccatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtggagatccagcccctgggcatcgcccccaccggctgcaagcgccgcgtggtggagggcggcggcggctccggcggcggcggctccgccgccggcctgggcgccctgttcctgggcttcctgggcgccgccggctccaccatgggcgccgcctccatgaccctgaccgtgcaggcccgccagctgctgtccggcatcgtgcagcagcagtccaacctgctgcgcgcccccgaggcccagcagcacatgctgcagccccccgtgtggggcatcaagcagctgcaggcccgcgtgctggccctggagcggtacctgaaggaccagcagctgctgggcatgtggggctgctccggcaagctgatctgctgcaccaacgtgccctggaacacctcctggtccaacaagtccgagaccgacatctgggacaacatgacctggatgcagtgggagcgcgagatctccaactacaccgagaccatctacaagctgctggaggactcccagaaccagcaggagaagaacgagcaggacctgctggccctggactcctggaactccctgtggaactggttctccatcaccaagtggctgtggtacatcaagatcttcatcatgatcgtgggcggcctgatcggcctgcgcatcgtgttcgccgtgctgtccatcgtgaaccgcgtgcgccagggcatctcccccctgtccctgcagaccctgacccccaacccccgcgagcccgaccgcctgcgcggcatcgaggaggagggcggcgagcaggaccgcgaccgctccatccgcctggtgtccggcttcctgcccatcgtgtgggacgacctgcgctccctgtgcctgttctcctaccaccgcctgcgcgacttcctgctgctggccgcccgcgtggtggagctgctgggccgctcctccctgcgcggcctgcagcgcggctgggaggtgctgaagtacctgggctccctggtgcagtactggggcctggagctgaagaagtccgccatctccctgttcgacaccctggccatcgccgtggccgagggcaccgaccgcatcatcgagctgatccagggcttctgccgcgccatccgcaacatccccacccgcatccgccagggcttcgaggcctccctgctgtaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 70 - DNA, 2982 bp, synthetic construct
  source 1..2982: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacatgaccaccgagctgcgcgacaagaagcagaaggtgtactccctgttctaccgcctggacgtggtgcagatcaacgagaaccagggcaaccgctccaacaactccaacaaggagtaccgcctgatcaactgcaacacctccgccgtgacccaggtgtgccccaagctgaccttcgagcccatccccatccactactgcgcccccgccggctacgccatcctgaagtgcaacaacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgaacacctccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgcgccatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtggagatccagcccctgggcatcgcccccaccggcgccaagcgccgcgtggtggagcgcgagaagcgcgccgccggcctgggcgccctgttcctgggcttcctgggcgccgccggctccaccatgggcgccgcctccatcaccctgaccgtgcaggcccgccagctgctgtccggcatcgtgcagcagcagtccaacctgctgcgcgccatcgaggcccagcagcacatgctgcagctgaccgtgtggggcatcaagcagctgcaggcccgcgtgctggccctggagcggtacctgaaggaccagcagctgctgggcatgtggggctgctccggcaagctgatctgcaccaccaacgtgccctggaacacctcctggtccaacaagtccgagaccgacatctgggacaacatgacctggatgcagtgggagcgcgagatctccaactacaccgagaccatctacaagctgctggaggactcccagaaccagcaggagcgcaacgagcaggacctgctggccctggactcctggaactccctgtggaactggttctccatcaccaagtggctgtggtacatcaagatcttcatcatgatcgtgggcggcctgatcggcctgcgcatcgtgttcgccgtgctgtccatcgtgaaccgcgtgcgccagggcatctcccccctgtccctgcagaccctgacccccaacccccgcgagcccgaccgcctgcgcggcatcgaggaggagggcggcgagcaggaccgcgaccgctccatccgcctggtgtccggcttcctgcccatcgtgtgggacgacctgcgctccctgtgcctgttctcctaccaccgcctgcgcgacttcctgctgctggccgcccgcgtggtggagctgctgggccgctcctccctgcgcggcctgcagcgcggctgggaggtgctgaagtacctgggctccctggtgcagtactggggcctggagctgaagaagtccgccatctccctgttcgacaccctggccatcgccgtggccgagggcaccgaccgcatcatcgagctgatccagggcttctgccgcgccatccgcaacatccccacccgcatccgccagggcttcgaggcctccctgctgtaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 71 - DNA, 3000 bp, synthetic construct
  source 1..3000: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacatgaccaccgagctgcgcgacaagaagcagaaggtgtactccctgttctaccgcctggacgtggtgcagatcaacgagaaccagggcaaccgctccaacaactccaacaaggagtaccgcctgatcaactgcaacacctccgccgtgacccaggtgtgccccaagctgaccttcgagcccatccccatccactactgcgcccccgccggctacgccatcctgaagtgcaacaacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgaacacctccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgcgccatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtggagatccagcccctgggcatcgcccccaccggctgcaagcgccgcgtggtggagggcggcggcggctccggcggcggcggctccgccgccggcctgggcgccctgttcctgggcttcctgggcgccgccggctccaccatgggcgccgcctccatcaccctgaccgtgcaggcccgccagctgctgtccggcatcgtgcagcagcagtccaacctgctgcgcgccatcgaggcccagcagcacatgctgcagctgaccgtgtggggcatcaagcagctgcaggcccgcgtgctggccctggagcggtacctgaaggaccagcagctgctgggcatgtggggctgctccggcaagctgatctgctgcaccaacgtgccctggaacacctcctggtccaacaagtccgagaccgacatctgggacaacatgacctggatgcagtgggagcgcgagatctccaactacaccgagaccatctacaagctgctggaggactcccagaaccagcaggagcgcaacgagcaggacctgctggccctggactcctggaactccctgtggaactggttctccatcaccaagtggctgtggtacatcaagatcttcatcatgatcgtgggcggcctgatcggcctgcgcatcgtgttcgccgtgctgtccatcgtgaaccgcgtgcgccagggcatctcccccctgtccctgcagaccctgacccccaacccccgcgagcccgaccgcctgcgcggcatcgaggaggagggcggcgagcaggaccgcgaccgctccatccgcctggtgtccggcttcctgcccatcgtgtgggacgacctgcgctccctgtgcctgttctcctaccaccgcctgcgcgacttcctgctgctggccgcccgcgtggtggagctgctgggccgctcctccctgcgcggcctgcagcgcggctgggaggtgctgaagtacctgggctccctggtgcagtactggggcctggagctgaagaagtccgccatctccctgttcgacaccctggccatcgccgtggccgagggcaccgaccgcatcatcgagctgatccagggcttctgccgcgccatccgcaacatccccacccgcatccgccagggcttcgaggcctccctgctgtaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 72 - DNA, 3000 bp, synthetic construct
  source 1..3000: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacatgaccaccgagctgcgcgacaagaagcagaaggtgtactccctgttctaccgcctggacgtggtgcagatcaacgagaaccagggcaaccgctccaacaactccaacaaggagtaccgcctgatcaactgcaacacctccgccgtgacccaggtgtgccccaagctgaccttcgagcccatccccatccactactgcgcccccgccggctacgccatcctgaagtgcaacaacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgaacacctccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgcgccatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtggagatccagcccctgggcatcgcccccaccggctgcaagcgccgcgtggtggagggcggcggcggctccggcggcggcggctccgccgccggcctgggcgccctgttcctgggcttcctgggcgccgccggctccaccatgggcgccgcctccatcaccctgaccgtgcaggcccgccagctgctgggctccgccggctccgccggctccggctccgccggctccggctccgccggctccggctccgccggctccaccgtgtggggcatcaagcagctgcaggcccgcgtgctggccctggagcggtacctgaaggaccagcagctgctgggcatgtggggctgctccggcaagctgatctgctgcaccaacgtgccctggaacacctcctggtccaacaagtccgagaccgacatctgggacaacatgacctggatgcagtgggagcgcgagatctccaactacaccgagaccatctacaagctgctggaggactcccagaaccagcaggagcgcaacgagcaggacctgctggccctggactcctggaactccctgtggaactggttctccatcaccaagtggctgtggtacatcaagatcttcatcatgatcgtgggcggcctgatcggcctgcgcatcgtgttcgccgtgctgtccatcgtgaaccgcgtgcgccagggcatctcccccctgtccctgcagaccctgacccccaacccccgcgagcccgaccgcctgcgcggcatcgaggaggagggcggcgagcaggaccgcgaccgctccatccgcctggtgtccggcttcctgcccatcgtgtgggacgacctgcgctccctgtgcctgttctcctaccaccgcctgcgcgacttcctgctgctggccgcccgcgtggtggagctgctgggccgctcctccctgcgcggcctgcagcgcggctgggaggtgctgaagtacctgggctccctggtgcagtactggggcctggagctgaagaagtccgccatctccctgttcgacaccctggccatcgccgtggccgagggcaccgaccgcatcatcgagctgatccagggcttctgccgcgccatccgcaacatccccacccgcatccgccagggcttcgaggcctccctgctgtaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 73 - DNA, 3000 bp, synthetic construct
  source 1..3000: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacatgaccaccgagctgcgcgacaagaagcagaaggtgtactccctgttctaccgcctggacgtggtgcagatcaacgagaaccagggcaaccgctccaacaactccaacaaggagtaccgcctgatcaactgcaacacctccgccgtgacccaggtgtgccccaagctgaccttcgagcccatccccatccactactgcgcccccgccggctacgccatcctgaagtgcaacaacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgaacacctccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgcgccatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtggagatccagcccctgggcatcgcccccaccggctgcaagcgccgcgtggtggagggcggcggcggctccggcggcggcggctccgccgccggcctgggcgccctgttcctgggcttcctgggcgccgccggctccaccatgggcgccgcctccatcaccctgaccgtgcaggcccgccagctgctgtccggcatcgtgcagcagcagtccaacctgctgcgcgcccccgaggcccagcagcacatgctgcagccccccgtgtggggcatcaagcagctgcaggcccgcgtgctggccctggagcggtacctgaaggaccagcagctgctgggcatgtggggctgctccggcaagctgatctgctgcaccaacgtgccctggaacacctcctggtccaacaagtccgagaccgacatctgggacaacatgacctggatgcagtgggagcgcgagatctccaactacaccgagaccatctacaagctgctggaggactcccagaaccagcaggagcgcaacgagcaggacctgctggccctggactcctggaactccctgtggaactggttctccatcaccaagtggctgtggtacatcaagatcttcatcatgatcgtgggcggcctgatcggcctgcgcatcgtgttcgccgtgctgtccatcgtgaaccgcgtgcgccagggcatctcccccctgtccctgcagaccctgacccccaacccccgcgagcccgaccgcctgcgcggcatcgaggaggagggcggcgagcaggaccgcgaccgctccatccgcctggtgtccggcttcctgcccatcgtgtgggacgacctgcgctccctgtgcctgttctcctaccaccgcctgcgcgacttcctgctgctggccgcccgcgtggtggagctgctgggccgctcctccctgcgcggcctgcagcgcggctgggaggtgctgaagtacctgggctccctggtgcagtactggggcctggagctgaagaagtccgccatctccctgttcgacaccctggccatcgccgtggccgagggcaccgaccgcatcatcgagctgatccagggcttctgccgcgccatccgcaacatccccacccgcatccgccagggcttcgaggcctccctgctgtaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 74 - DNA, 3000 bp, synthetic construct
  source 1..3000: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacatgaccaccgagctgcgcgacaagaagcagaaggtgtactccctgttctaccgcctggacgtggtgcagatcaacgagaaccagggcaaccgctccaacaactccaacaaggagtaccgcctgatcaactgcaacacctccgccgtgacccaggtgtgccccaagctgaccttcgagcccatccccatccactactgcgcccccgccggctacgccatcctgaagtgcaacaacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgaacacctccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgcgccatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtggagatccagcccctgggcatcgcccccaccggctgcaagcgccgcgtggtggagggcggcggcggctccggcggcggcggctccgccgccggcctgggcgccctgttcctgggcttcctgggcgccgccggctccaccatgggcgccgcctccatgaccctgaccgtgcaggcccgccagctgctgggctccgccggctccgccggctccggctccgccggctccggctccgccggctccggctccgccggctccaccgtgtggggcatcaagcagctgcaggcccgcgtgctggccctggagcggtacctgaaggaccagcagctgctgggcatgtggggctgctccggcaagctgatctgctgcaccaacgtgccctggaacacctcctggtccaacaagtccgagaccgacatctgggacaacatgacctggatgcagtgggagcgcgagatctccaactacaccgagaccatctacaagctgctggaggactcccagaaccagcaggagaagaacgagcaggacctgctggccctggactcctggaactccctgtggaactggttctccatcaccaagtggctgtggtacatcaagatcttcatcatgatcgtgggcggcctgatcggcctgcgcatcgtgttcgccgtgctgtccatcgtgaaccgcgtgcgccagggcatctcccccctgtccctgcagaccctgacccccaacccccgcgagcccgaccgcctgcgcggcatcgaggaggagggcggcgagcaggaccgcgaccgctccatccgcctggtgtccggcttcctgcccatcgtgtgggacgacctgcgctccctgtgcctgttctcctaccaccgcctgcgcgacttcctgctgctggccgcccgcgtggtggagctgctgggccgctcctccctgcgcggcctgcagcgcggctgggaggtgctgaagtacctgggctccctggtgcagtactggggcctggagctgaagaagtccgccatctccctgttcgacaccctggccatcgccgtggccgagggcaccgaccgcatcatcgagctgatccagggcttctgccgcgccatccgcaacatccccacccgcatccgccagggcttcgaggcctccctgctgtaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 75 - DNA, 3000 bp, synthetic construct
  source 1..3000: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacatgaccaccgagctgcgcgacaagaagcagaaggtgtactccctgttctaccgcctggacgtggtgcagatcaacgagaaccagggcaaccgctccaacaactccaacaaggagtaccgcctgatcaactgcaacacctccgccgtgacccaggtgtgccccaagctgaccttcgagcccatccccatccactactgcgcccccgccggctacgccatcctgaagtgcaacaacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgaacacctccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgcgccatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtggagatccagcccctgggcatcgcccccaccggctgcaagcgccgcgtggtggagggcggcggcggctccggcggcggcggctccgccgccggcctgggcgccctgttcctgggcttcctgggcgccgccggctccaccatgggcgccgcctccatgaccctgaccgtgcaggcccgccagctgctgtccggcatcgtgcagcagcagtccaacctgctgcgcgcccccgaggcccagcagcacatgctgcagccccccgtgtggggcatcaagcagctgcaggcccgcgtgctggccctggagcggtacctgaaggaccagcagctgctgggcatgtggggctgctccggcaagctgatctgctgcaccaacgtgccctggaacacctcctggtccaacaagtccgagaccgacatctgggacaacatgacctggatgcagtgggagcgcgagatctccaactacaccgagaccatctacaagctgctggaggactcccagaaccagcaggagaagaacgagcaggacctgctggccctggactcctggaactccctgtggaactggttctccatcaccaagtggctgtggtacatcaagatcttcatcatgatcgtgggcggcctgatcggcctgcgcatcgtgttcgccgtgctgtccatcgtgaaccgcgtgcgccagggcatctcccccctgtccctgcagaccctgacccccaacccccgcgagcccgaccgcctgcgcggcatcgaggaggagggcggcgagcaggaccgcgaccgctccatccgcctggtgtccggcttcctgcccatcgtgtgggacgacctgcgctccctgtgcctgttctcctaccaccgcctgcgcgacttcctgctgctggccgcccgcgtggtggagctgctgggccgctcctccctgcgcggcctgcagcgcggctgggaggtgctgaagtacctgggctccctggtgcagtactggggcctggagctgaagaagtccgccatctccctgttcgacaccctggccatcgccgtggccgagggcaccgaccgcatcatcgagctgatccagggcttctgccgcgccatccgcaacatccccacccgcatccgccagggcttcgaggcctccctgctgtaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 76 - DNA, 2373 bp, synthetic construct
  source 1..2373: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgcccgcgcctacgagaaggaggtgcacaacgtgtgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacaccaccaccgagatccgcgacaaggagaagaaggagtacgccctgttctacaagcccgacatcgtgcccctgtccgagaccaacaacacctccgagtaccgcctgatcaactgcaacacctccgcctgcacccaggcctgccccaaggtgaccttcgagcccatccccatccactactgcgcccccgccggctacgccatcctgaagtgcaacgacgagaccttcaacggcaccggcccctgctccaacgtgtccaccgtgcagtgcacccacggcatccgccccgtggtgtccacccagctgctgctgaacggctccctggccgagaaggagatcgtgatccgctccgagaacctgaccaacaacgccaagatcatcatcgtgcacctgcacacccccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgctgcatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggactaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 77 - DNA, 2373 bp, synthetic construct
  source 1..2373: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgcccgcgcctacgagaaggaggtgcacaacgtgtgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacaccaccaccgagatccgcgacaaggagaagaaggagtacgccctgttctacaagcccgacatcgtgcccctgtccgagaccaacaacacctccgagtaccgcctgatcaactgcaacacctccgcctgcacccaggcctgccccaaggtgaccttcgagcccatccccatccactactgcgcccccgccggctacgccatcctgaagtgcaacgacgagaccttcaacggcaccggcccctgctccaacgtgtccaccgtgcagtgcacccacggcatccgccccgtggtgtccacccagctgctgctgaacggctccctggccgagaaggagatcgtgatccgctccgagaacctgaccaacaacgccaagatcatcatcgtgcacctgcacacccccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgctgcatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagccccccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggactaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 78 - DNA, 2373 bp, synthetic construct
  source 1..2373: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgcccgcgcctacgagaaggaggtgcacaacgtgtgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacaccaccaccgagatccgcgacaagaagaagaaggagtacgccctgttctacaagcccgacatcgtgcccctgtccgagaccaacaacacctccgagtaccgcctgatcaactgcaacacctccgcctgcacccaggcctgccccaaggtgaccttcgagcccatccccatccactactgcgcccccgccggctacgccatcctgaagtgcaacgacgagaccttcaacggcaccggcccctgctccaacgtgtccaccgtgcagtgcacccacggcatccgccccgtggtgtccacccagctgctgctgaacggctccctggccgagaaggagatcgtgatccgctccgagaacctgaccaacaacgccaagatcatcatcgtgcacctgcacacccccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgctgcatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagccccccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggactaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 79 - DNA, 2367 bp, synthetic construct
  source 1..2367: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacaccaccaccgagatccgcgacaagaagaagaaggagtacgccctgttctacaagcccgacatcgtgcccctgtccgagaccaacaacacctccgagtaccgcctgatcaactgcaacacctccgccgtgacccaggtgtgccccaagctgaccttcgagcccatccccatccactactgcgcccccgccggctacgccatcctgaagtgcaacgacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgcacacccccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgcgccatgtacgccccccccatcgccggcaacatcacctgccgctccaacatcaccggcctgctgctgacccgcgacggcggcaccaactccaacgagaccgagaccttccgccccggcggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgtccctgggcttcctgggcgccgccggctccaccatgggcgccgcctccatgaccctgaccgtgcaggcccgcaacctgctgtccggcatcgtgcagcagcagtccaacctgctgcgcgcccccgagccccagcagcacctgctgaaggacacccactggggcatcaagcagctgcaggcccgcgtgctggccgtggagcactacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggactaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 80 - DNA, 2367 bp, synthetic construct
  source 1..2367: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacaccaccaccgagatccgcgacaagaagaagaaggagtacgccctgttctacaagcccgacatcgtgcccctgtccgagaccaacaacacctccgagtaccgcctgatcaactgcaacacctccgcctgcacccaggtgtgccccaagctgaccttcgagcccatccccatccactactgcgcccccgccggctacgccatcctgaagtgcaacgacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgcacacccccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgctgcatgtacgccccccccatcgccggcaacatcacctgccgctccaacatcaccggcctgctgctgacccgcgacggcggcaccaactccaacgagaccgagaccttccgccccggcggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgtccctgggcttcctgggcgccgccggctccaccatgggcgccgcctccatgaccctgaccgtgcaggcccgcaacctgctgtccggcatcgtgcagcagcagtccaacctgctgcgcgcccccgagccccagcagcacctgctgaaggacacccactggggcatcaagcagctgcaggcccgcgtgctggccgtggagcactacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggactaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 81 - DNA, 2982 bp, synthetic construct
  source 1..2982: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacaccaccaccgagctgcgcgacaagaagaagaaggagtacgccctgttctacaagcccgacatcgtgcccctgtccgagaccaacaacacctccgagtaccgcctgatcaactgcaacacctccgcctgcacccaggtgtgccccaagctgaccttcgagcccatccccatccactactgcgcccccgccggctacgccatcctgaagtgcaacaacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgaacacctccgtggagatcgtgtgcacccgccccaacaacaacacccgcaagtccgtgcgcatcggccccggccagtggttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgctgcatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtggagatccagcccctgggcatcgcccccaccggctgcaagcgccgcgtggtggagggcggcggcggctccggcggcggcggctccgccgccggcctgggcgccctgttcctgggcttcctgggcgccgccggctccaccatgggcgccgcctccatgaccctgaccgtgcaggcccgccagctgctgggctccgccggctccgccggctccggctccgccggctccggctccgccggctccggctccgccggctccaccgtgtggggcatcaagcagctgcaggcccgcgtgctggccctggagcgctacctgaaggaccagcagctgctgggcatgtggggctgctccggcaagctgatctgctgcaccaacgtgccctggaacacctcctggtccaacaagtccgagaccgacatctgggacaacatgacctggatgcagtgggagcgcgagatctccaactacaccgagaccatctacaagctgctggaggactcccagaaccagcaggagaagaacgagcaggacctgctggccctggactcctggaactccctgtggaactggttctccatcaccaagtggctgtggtacatcaagatcttcatcatgatcgtgggcggcctgatcggcctgcgcatcgtgttcgccgtgctgtccatcgtgaaccgcgtgcgccagggcatctcccccctgtccctgcagaccctgacccccaacccccgcgagcccgaccgcctgcgcggcatcgaggaggagggcggcgagcaggaccgcgaccgctccatccgcctggtgtccggcttcctgcccatcgtgtgggacgacctgcgctccctgtgcctgttctcctaccaccgcctgcgcgacttcctgctgctggccgcccgcgtggtggagctgctgggccgctcctccctgcgcggcctgcagcgcggctgggaggtgctgaagtacctgggctccctggtgcagtactggggcctggagctgaagaagtccgccatctccctgttcgacaccctggccatcgccgtggccgagggcaccgaccgcatcatcgagctgatccagggcttctgccgcgccatccgcaacatccccacccgcatccgccagggcttcgaggcctccctgctgtaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 82 - DNA, 2982 bp, synthetic construct
  source 1..2982: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacaccaccaccgagctgcgcgacaagaagaagaaggagtacgccctgttctacaagcccgacatcgtgcccctgtccgagaccaacaacacctccgagtaccgcctgatcaactgcaacacctccgcctgcaccatggtgtgccccaagctgaccttcgagcccatccccatccactactgcgcccccgccggctacgccatcctgaagtgcaacaacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgaacacctccgtggagatcgtgtgcacccgccccctgaacctgacccgcaagtccgtgcgcatcggccccggccagtggttctacgccatgggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagatgatcatcaacatgtggcagggcgtgggccgctgcatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtggagatccagcccctgggcatcgcccccaccggctgcaagcgccgcgtggtggagggcggcggcggctccggcggcggcggctccgccgccggcctgggcgccctgttcctgggcttcctgggcgccgccggctccaccatgggcgccgcctccatgaccctgaccgtgcaggcccgccagctgctgggctccgccggctccgccggctccggctccgccggctccggctccgccggctccggctccgccggctccaccgtgtggggcatcaagcagctgcaggcccgcgtgctggccctggagcgctacctgaaggaccagcagctgctgggcatgtggggctgctccggcaagctgatctgctgcaccaacgtgccctggaacacctcctggtccaacaagtccgagaccgacatctgggacaacatgacctggatgcagtgggagcgcgagatctccaactacaccgagaccatctacaagctgctggaggactcccagaaccagcaggagaagaacgagcaggacctgctggccctggactcctggaactccctgtggaactggttctccatcaccaagtggctgtggtacatcaagatcttcatcatgatcgtgggcggcctgatcggcctgcgcatcgtgttcgccgtgctgtccatcgtgaaccgcgtgcgccagggcatctcccccctgtccctgcagaccctgacccccaacccccgcgagcccgaccgcctgcgcggcatcgaggaggagggcggcgagcaggaccgcgaccgctccatccgcctggtgtccggcttcctgcccatcgtgtgggacgacctgcgctccctgtgcctgttctcctaccaccgcctgcgcgacttcctgctgctggccgcccgcgtggtggagctgctgggccgctcctccctgcgcggcctgcagcgcggctgggaggtgctgaagtacctgggctccctggtgcagtactggggcctggagctgaagaagtccgccatctccctgttcgacaccctggccatcgccgtggccgagggcaccgaccgcatcatcgagctgatccagggcttctgccgcgccatccgcaacatccccacccgcatccgccagggcttcgaggcctccctgctgtaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 83 - DNA, 2982 bp, synthetic construct
  source 1..2982: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacaccaccaccgagctgcgcgacaagaagaagaaggagtacgccctgttctacaagcccgacatcgtgcccctgtccgagaccaacaacacctccgagtaccgcctgatcaactgcaacacctccgccgtgaccatggtgtgccccaagctgaccttcgagcccatccccatccactactgcgcccccgccggctacgccatcctgaagtgcaacaacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgaacacctccgtggagatcgtgtgcacccgccccctgaacctgacccgcaagtccgtgcgcatcggccccggccagaccttctacgccatgggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagatgatcatcaacatgtggcagggcgtgggccgcgccatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtggagatccagcccctgggcatcgcccccaccggctgcaagcgccgcgtggtggagggcggcggcggctccggcggcggcggctccgccgccggcctgggcgccctgttcctgggcttcctgggcgccgccggctccaccatgggcgccgcctccatgaccctgaccgtgcaggcccgccagctgctgggctccgccggctccgccggctccggctccgccggctccggctccgccggctccggctccgccggctccaccgtgtggggcatcaagcagctgcaggcccgcgtgctggccctggagcgctacctgaaggaccagcagctgctgggcatgtggggctgctccggcaagctgatctgctgcaccaacgtgccctggaacacctcctggtccaacaagtccgagaccgacatctgggacaacatgacctggatgcagtgggagcgcgagatctccaactacaccgagaccatctacaagctgctggaggactcccagaaccagcaggagaagaacgagcaggacctgctggccctggactcctggaactccctgtggaactggttctccatcaccaagtggctgtggtacatcaagatcttcatcatgatcgtgggcggcctgatcggcctgcgcatcgtgttcgccgtgctgtccatcgtgaaccgcgtgcgccagggcatctcccccctgtccctgcagaccctgacccccaacccccgcgagcccgaccgcctgcgcggcatcgaggaggagggcggcgagcaggaccgcgaccgctccatccgcctggtgtccggcttcctgcccatcgtgtgggacgacctgcgctccctgtgcctgttctcctaccaccgcctgcgcgacttcctgctgctggccgcccgcgtggtggagctgctgggccgctcctccctgcgcggcctgcagcgcggctgggaggtgctgaagtacctgggctccctggtgcagtactggggcctggagctgaagaagtccgccatctccctgttcgacaccctggccatcgccgtggccgagggcaccgaccgcatcatcgagctgatccagggcttctgccgcgccatccgcaacatccccacccgcatccgccagggcttcgaggcctccctgctgtaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 84 - DNA, 2982 bp, synthetic construct
  source 1..2982: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacaccaccaccgagctgcgcgacaagaagaagaaggagtacgccctgttctacaagcccgacatcgtgcccctgtccgagaccaacaacacctccgagtaccgcctgatcaactgcaacacctccgcctgcacccaggtgtgccccaagctgaccttcgagcccatccccatccactactgcgcccccgccggctacgccatcctgaagtgcaacaacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgaacacctccgtggagatcgtgtgcacccgccccaacaacaacacccgcaagtccgtgcgcatcggccccggccagtggttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgctgcatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtggagatccagcccctgggcatcgcccccaccggctgcaagcgccgcgtggtggagggcggcggcggctccggcggcggcggctccgccgccggcctgggcgccctgttcctgggcttcctgggcgccgccggctccaccatgggcgccgcctccatgaccctgaccgtgcaggcccgccagctgctgggctccgccggctccgccggctccggctccgccggctccggctccgccggctccggctccgccggctccaccgtgtggggcatcaagcagctgcaggcccgcgtgctggccctggagcgctacctgaaggaccagcagctgctgggcatgtggggctgctccggcaagctgatctgctgcaccaacgtgccctggaacacctcctggtccaacaagtccgagaccgacatctgggacaacatgacctggatgcagtgggagcgcgagatctccaactacaccgagaccatctacaagctgctggaggactcccagaaccagcaggagaagaacgagcaggacctgctggccctggactcctggaactccctgtggaactggttctccatcaccaagtggctgtggtacatcaagatcttcatcatgatcgtgggcggcctgatcggcctgcgcatcgtgttcgccgtgctgtccatcgtgaaccgcgtgcgccagggcatctcccccctgtccctgcagaccctgacccccaacccccgcgagcccgaccgcctgcgcggcatcgaggaggagggcggcgagcaggaccgcgaccgctccatccgcctggtgtccggcttcctgcccatcgtgtgggacgacctgcgctccctgtgcctgttctcctaccaccgcctgcgcgacttcctgctgctggccgcccgcgtggtggagctgctgggccgctcctccctgcgcggcctgcagcgcggctgggaggtgctgaagtacctgggctccctggtgcagtactggggcctggagctgaagaagtccgccatctccctgttcgacaccctggccatcgccgtggccgagggcaccgaccgcatcatcgagctgatccagggcttctgccgcgccatccgcaacatccccacccgcatccgccagggcttcgaggcctccgccgcctaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 85 - DNA, 2982 bp, synthetic construct
  source 1..2982: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacaccaccaccgagctgcgcgacaagaagaagaaggagtacgccctgttctacaagcccgacatcgtgcccctgtccgagaccaacaacacctccgagtaccgcctgatcaactgcaacacctccgcctgcaccatggtgtgccccaagctgaccttcgagcccatccccatccactactgcgcccccgccggctacgccatcctgaagtgcaacaacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgaacacctccgtggagatcgtgtgcacccgccccctgaacctgacccgcaagtccgtgcgcatcggccccggccagtggttctacgccatgggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagatgatcatcaacatgtggcagggcgtgggccgctgcatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtggagatccagcccctgggcatcgcccccaccggctgcaagcgccgcgtggtggagggcggcggcggctccggcggcggcggctccgccgccggcctgggcgccctgttcctgggcttcctgggcgccgccggctccaccatgggcgccgcctccatgaccctgaccgtgcaggcccgccagctgctgggctccgccggctccgccggctccggctccgccggctccggctccgccggctccggctccgccggctccaccgtgtggggcatcaagcagctgcaggcccgcgtgctggccctggagcgctacctgaaggaccagcagctgctgggcatgtggggctgctccggcaagctgatctgctgcaccaacgtgccctggaacacctcctggtccaacaagtccgagaccgacatctgggacaacatgacctggatgcagtgggagcgcgagatctccaactacaccgagaccatctacaagctgctggaggactcccagaaccagcaggagaagaacgagcaggacctgctggccctggactcctggaactccctgtggaactggttctccatcaccaagtggctgtggtacatcaagatcttcatcatgatcgtgggcggcctgatcggcctgcgcatcgtgttcgccgtgctgtccatcgtgaaccgcgtgcgccagggcatctcccccctgtccctgcagaccctgacccccaacccccgcgagcccgaccgcctgcgcggcatcgaggaggagggcggcgagcaggaccgcgaccgctccatccgcctggtgtccggcttcctgcccatcgtgtgggacgacctgcgctccctgtgcctgttctcctaccaccgcctgcgcgacttcctgctgctggccgcccgcgtggtggagctgctgggccgctcctccctgcgcggcctgcagcgcggctgggaggtgctgaagtacctgggctccctggtgcagtactggggcctggagctgaagaagtccgccatctccctgttcgacaccctggccatcgccgtggccgagggcaccgaccgcatcatcgagctgatccagggcttctgccgcgccatccgcaacatccccacccgcatccgccagggcttcgaggcctccgccgcctaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 86 - DNA, 2982 bp, synthetic construct
  source 1..2982: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacaccaccaccgagctgcgcgacaagaagaagaaggagtacgccctgttctacaagcccgacatcgtgcccctgtccgagaccaacaacacctccgagtaccgcctgatcaactgcaacacctccgccgtgaccatggtgtgccccaagctgaccttcgagcccatccccatccactactgcgcccccgccggctacgccatcctgaagtgcaacaacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgaacacctccgtggagatcgtgtgcacccgccccctgaacctgacccgcaagtccgtgcgcatcggccccggccagaccttctacgccatgggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagatgatcatcaacatgtggcagggcgtgggccgcgccatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtggagatccagcccctgggcatcgcccccaccggctgcaagcgccgcgtggtggagggcggcggcggctccggcggcggcggctccgccgccggcctgggcgccctgttcctgggcttcctgggcgccgccggctccaccatgggcgccgcctccatgaccctgaccgtgcaggcccgccagctgctgggctccgccggctccgccggctccggctccgccggctccggctccgccggctccggctccgccggctccaccgtgtggggcatcaagcagctgcaggcccgcgtgctggccctggagcgctacctgaaggaccagcagctgctgggcatgtggggctgctccggcaagctgatctgctgcaccaacgtgccctggaacacctcctggtccaacaagtccgagaccgacatctgggacaacatgacctggatgcagtgggagcgcgagatctccaactacaccgagaccatctacaagctgctggaggactcccagaaccagcaggagaagaacgagcaggacctgctggccctggactcctggaactccctgtggaactggttctccatcaccaagtggctgtggtacatcaagatcttcatcatgatcgtgggcggcctgatcggcctgcgcatcgtgttcgccgtgctgtccatcgtgaaccgcgtgcgccagggcatctcccccctgtccctgcagaccctgacccccaacccccgcgagcccgaccgcctgcgcggcatcgaggaggagggcggcgagcaggaccgcgaccgctccatccgcctggtgtccggcttcctgcccatcgtgtgggacgacctgcgctccctgtgcctgttctcctaccaccgcctgcgcgacttcctgctgctggccgcccgcgtggtggagctgctgggccgctcctccctgcgcggcctgcagcgcggctgggaggtgctgaagtacctgggctccctggtgcagtactggggcctggagctgaagaagtccgccatctccctgttcgacaccctggccatcgccgtggccgagggcaccgaccgcatcatcgagctgatccagggcttctgccgcgccatccgcaacatccccacccgcatccgccagggcttcgaggcctccgccgcctaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 87 - DNA, 2658 bp, synthetic construct
  source 1..2658: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacaccaccaccgagctgcgcgacaagaagaagaaggagtacgccctgttctacaagcccgacatcgtgcccctgtccgagaccaacaacacctccgagtaccgcctgatcaactgcaacacctccgcctgcacccaggtgtgccccaagctgaccttcgagcccatccccatccactactgcgcccccgccggctacgccatcctgaagtgcaacaacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgaacacctccgtggagatcgtgtgcacccgccccaacaacaacacccgcaagtccgtgcgcatcggccccggccagtggttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgctgcatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtggagatccagcccctgggcatcgcccccaccggctgcaagcgccgcgtggtggagggcggcggcggctccggcggcggcggctccgccgccggcctgggcgccctgttcctgggcttcctgggcgccgccggctccaccatgggcgccgcctccatgaccctgaccgtgcaggcccgccagctgctgggctccgccggctccgccggctccggctccgccggctccggctccgccggctccggctccgccggctccaccgtgtggggcatcaagcagctgcaggcccgcgtgctggccctggagcgctacctgaaggaccagcagctgctgggcatgtggggctgctccggcaagctgatctgctgcaccaacgtgccctggaacacctcctggtccaacaagtccgagaccgacatctgggacaacatgacctggatgcagtgggagcgcgagatctccaactacaccgagaccatctacaagctgctggaggactcccagaaccagcaggagaagaacgagcaggacctgctggccctggactcctggaactccctgtggaactggttctccatcaccaagtggctgtggtacatcaagatcttcatcatgatcgtgggcggcctgatcggcctgcgcatcgtgttcgccgtgctgtccatcgtgaaccgcgtgcgccagggcatctcccccctgtccctgcagaccctgacccccaacccccgcgagcccgaccgcctgcgcggcatcgaggaggagggcggcgagcaggaccgcgaccgctccatccgcctggtgtccggcttcctgcccatctaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 88 - DNA, 2658 bp, synthetic construct
  source 1..2658: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacaccaccaccgagctgcgcgacaagaagaagaaggagtacgccctgttctacaagcccgacatcgtgcccctgtccgagaccaacaacacctccgagtaccgcctgatcaactgcaacacctccgcctgcaccatggtgtgccccaagctgaccttcgagcccatccccatccactactgcgcccccgccggctacgccatcctgaagtgcaacaacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgaacacctccgtggagatcgtgtgcacccgccccctgaacctgacccgcaagtccgtgcgcatcggccccggccagtggttctacgccatgggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagatgatcatcaacatgtggcagggcgtgggccgctgcatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtggagatccagcccctgggcatcgcccccaccggctgcaagcgccgcgtggtggagggcggcggcggctccggcggcggcggctccgccgccggcctgggcgccctgttcctgggcttcctgggcgccgccggctccaccatgggcgccgcctccatgaccctgaccgtgcaggcccgccagctgctgggctccgccggctccgccggctccggctccgccggctccggctccgccggctccggctccgccggctccaccgtgtggggcatcaagcagctgcaggcccgcgtgctggccctggagcgctacctgaaggaccagcagctgctgggcatgtggggctgctccggcaagctgatctgctgcaccaacgtgccctggaacacctcctggtccaacaagtccgagaccgacatctgggacaacatgacctggatgcagtgggagcgcgagatctccaactacaccgagaccatctacaagctgctggaggactcccagaaccagcaggagaagaacgagcaggacctgctggccctggactcctggaactccctgtggaactggttctccatcaccaagtggctgtggtacatcaagatcttcatcatgatcgtgggcggcctgatcggcctgcgcatcgtgttcgccgtgctgtccatcgtgaaccgcgtgcgccagggcatctcccccctgtccctgcagaccctgacccccaacccccgcgagcccgaccgcctgcgcggcatcgaggaggagggcggcgagcaggaccgcgaccgctccatccgcctggtgtccggcttcctgcccatctaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 89 - DNA, 2658 bp, synthetic construct
  source 1..2658: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacaccaccaccgagctgcgcgacaagaagaagaaggagtacgccctgttctacaagcccgacatcgtgcccctgtccgagaccaacaacacctccgagtaccgcctgatcaactgcaacacctccgccgtgaccatggtgtgccccaagctgaccttcgagcccatccccatccactactgcgcccccgccggctacgccatcctgaagtgcaacaacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgaacacctccgtggagatcgtgtgcacccgccccctgaacctgacccgcaagtccgtgcgcatcggccccggccagaccttctacgccatgggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagatgatcatcaacatgtggcagggcgtgggccgcgccatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtggagatccagcccctgggcatcgcccccaccggctgcaagcgccgcgtggtggagggcggcggcggctccggcggcggcggctccgccgccggcctgggcgccctgttcctgggcttcctgggcgccgccggctccaccatgggcgccgcctccatgaccctgaccgtgcaggcccgccagctgctgggctccgccggctccgccggctccggctccgccggctccggctccgccggctccggctccgccggctccaccgtgtggggcatcaagcagctgcaggcccgcgtgctggccctggagcgctacctgaaggaccagcagctgctgggcatgtggggctgctccggcaagctgatctgctgcaccaacgtgccctggaacacctcctggtccaacaagtccgagaccgacatctgggacaacatgacctggatgcagtgggagcgcgagatctccaactacaccgagaccatctacaagctgctggaggactcccagaaccagcaggagaagaacgagcaggacctgctggccctggactcctggaactccctgtggaactggttctccatcaccaagtggctgtggtacatcaagatcttcatcatgatcgtgggcggcctgatcggcctgcgcatcgtgttcgccgtgctgtccatcgtgaaccgcgtgcgccagggcatctcccccctgtccctgcagaccctgacccccaacccccgcgagcccgaccgcctgcgcggcatcgaggaggagggcggcgagcaggaccgcgaccgctccatccgcctggtgtccggcttcctgcccatctaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 90 - DNA, 2529 bp, synthetic construct
  source 1..2529: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacaccaccaccgagctgcgcgacaagaagaagaaggagtacgccctgttctacaagcccgacatcgtgcccctgtccgagaccaacaacacctccgagtaccgcctgatcaactgcaacacctccgcctgcacccaggtgtgccccaagctgaccttcgagcccatccccatccactactgcgcccccgccggctacgccatcctgaagtgcaacaacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgaacacctccgtggagatcgtgtgcacccgccccaacaacaacacccgcaagtccgtgcgcatcggccccggccagtggttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgctgcatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtggagatccagcccctgggcatcgcccccaccggctgcaagcgccgcgtggtggagggcggcggcggctccggcggcggcggctccgccgccggcctgggcgccctgttcctgggcttcctgggcgccgccggctccaccatgggcgccgcctccatgaccctgaccgtgcaggcccgccagctgctgggctccgccggctccgccggctccggctccgccggctccggctccgccggctccggctccgccggctccaccgtgtggggcatcaagcagctgcaggcccgcgtgctggccctggagcgctacctgaaggaccagcagctgctgggcatgtggggctgctccggcaagctgatctgctgcaccaacgtgccctggaacacctcctggtccaacaagtccgagaccgacatctgggacaacatgacctggatgcagtgggagcgcgagatctccaactacaccgagaccatctacaagctgctggaggactcccagaaccagcaggagaagaacgagcaggacctgctggccctggactcctggaactccctgtggaactggttctccatcaccaagtggctgtggtacatcaagatcttcatcatgatcgtgggcggcctgatcggcctgcgcatcgtgttcgccgtgctgtccatcgtgaaccgcgtgcgccagggcatctaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 91 - DNA, 2529 bp, synthetic construct
  source 1..2529: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacaccaccaccgagctgcgcgacaagaagaagaaggagtacgccctgttctacaagcccgacatcgtgcccctgtccgagaccaacaacacctccgagtaccgcctgatcaactgcaacacctccgcctgcaccatggtgtgccccaagctgaccttcgagcccatccccatccactactgcgcccccgccggctacgccatcctgaagtgcaacaacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgaacacctccgtggagatcgtgtgcacccgccccctgaacctgacccgcaagtccgtgcgcatcggccccggccagtggttctacgccatgggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagatgatcatcaacatgtggcagggcgtgggccgctgcatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtggagatccagcccctgggcatcgcccccaccggctgcaagcgccgcgtggtggagggcggcggcggctccggcggcggcggctccgccgccggcctgggcgccctgttcctgggcttcctgggcgccgccggctccaccatgggcgccgcctccatgaccctgaccgtgcaggcccgccagctgctgggctccgccggctccgccggctccggctccgccggctccggctccgccggctccggctccgccggctccaccgtgtggggcatcaagcagctgcaggcccgcgtgctggccctggagcgctacctgaaggaccagcagctgctgggcatgtggggctgctccggcaagctgatctgctgcaccaacgtgccctggaacacctcctggtccaacaagtccgagaccgacatctgggacaacatgacctggatgcagtgggagcgcgagatctccaactacaccgagaccatctacaagctgctggaggactcccagaaccagcaggagaagaacgagcaggacctgctggccctggactcctggaactccctgtggaactggttctccatcaccaagtggctgtggtacatcaagatcttcatcatgatcgtgggcggcctgatcggcctgcgcatcgtgttcgccgtgctgtccatcgtgaaccgcgtgcgccagggcatctaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 92 - DNA, 2529 bp, synthetic construct
  source 1..2529: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacaccaccaccgagctgcgcgacaagaagaagaaggagtacgccctgttctacaagcccgacatcgtgcccctgtccgagaccaacaacacctccgagtaccgcctgatcaactgcaacacctccgccgtgaccatggtgtgccccaagctgaccttcgagcccatccccatccactactgcgcccccgccggctacgccatcctgaagtgcaacaacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgaacacctccgtggagatcgtgtgcacccgccccctgaacctgacccgcaagtccgtgcgcatcggccccggccagaccttctacgccatgggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagatgatcatcaacatgtggcagggcgtgggccgcgccatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtggagatccagcccctgggcatcgcccccaccggctgcaagcgccgcgtggtggagggcggcggcggctccggcggcggcggctccgccgccggcctgggcgccctgttcctgggcttcctgggcgccgccggctccaccatgggcgccgcctccatgaccctgaccgtgcaggcccgccagctgctgggctccgccggctccgccggctccggctccgccggctccggctccgccggctccggctccgccggctccaccgtgtggggcatcaagcagctgcaggcccgcgtgctggccctggagcgctacctgaaggaccagcagctgctgggcatgtggggctgctccggcaagctgatctgctgcaccaacgtgccctggaacacctcctggtccaacaagtccgagaccgacatctgggacaacatgacctggatgcagtgggagcgcgagatctccaactacaccgagaccatctacaagctgctggaggactcccagaaccagcaggagaagaacgagcaggacctgctggccctggactcctggaactccctgtggaactggttctccatcaccaagtggctgtggtacatcaagatcttcatcatgatcgtgggcggcctgatcggcctgcgcatcgtgttcgccgtgctgtccatcgtgaaccgcgtgcgccagggcatctaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 93 - DNA, 2394 bp, synthetic construct
  source 1..2394: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccaagggcaagctgtgggtgaccgtgtactacggcgtgcccgtgtggaaggaggccaagaccaccctgttctgcgcctccgacgcccgcgcctacgagaaggaggtgcacaacatctgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacaccaccaccgagctgcgcgacaagaagaagaaggagtacgccctgttctacaagcccgacatcgtgcccctgtccgagaccaacaacacctccgagtaccgcctgatcaactgcaacacctccgccgtgacccaggtgtgccccaagctgaccttcgagcccatccccatccactactgcgcccccgccggctacgccatcctgaagtgcaacaacgagaccttcaacggcaccggcccctgctccaacgtgtccaccgtgcagtgcacccacggcatccgccccgtgctgtccacccagctgctgctgaacggctccctggccgagaaggagatcgtgatccgctccgagaacctgaccaacaacgccaagatcatcatcgtgcacctgaacacctccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgcgccatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtggagatccagcccctgggcatcgcccccaccggctgcaagcgccgcgtggtggagggcggcggcggctccggcggcggcggctccgccgccggcctgggcgccctgttcctgggcttcctgggcgccgccggctccaccatgggcgccgcctccatgaccctgaccgtgcaggcccgccagctgctgggctccgccggctccgccggctccggctccgccggctccggctccgccggctccggctccgccggctccaccgtgtggggcatcaagcagctgcaggcccgcgtgctggccctggagcgctacctgaaggaccagcagctgctgggcatgtggggctgctccggcaagctgatctgctgcaccaacgtgccctggaacacctcctggtccaacaagtccgagaccgacatctgggacaacatgacctggatgaactggtcccgcgagatctccaactacaccgagaccatctacaagctgctggaggactcccagaaccagcaggagaagaacaaccagtccctgctggccctggacaacggctcctaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 94 - DNA, 2391 bp, synthetic construct
  source 1..2391: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatgcccatgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgcccgcgcctacgagaaggaggtgcacaacgtgtgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgcaccaactacgcccccaagctgcgctccatgatgcgcggcgagatcaagaactgctccttcaacatgaccaccgagctgcgcgacaagaagcagaaggtgtactccctgttctaccgcctggacgtggtgcagatcaacgagaaccagggcaaccgctccaacaactccaacaaggagtaccgcctgatcaactgcaacacctccgcctgcacccaggcctgccccaaggtgaccttcgagcccatccccatccactactgcgcccccgccggctacgccatcctgaagtgcaacaacgagaccttcaacggcaccggcccctgctccaacgtgtccaccgtgcagtgcacccacggcatccgccccgtggtgtccacccagctgctgctgaacggctccctggccgagaaggagatcgtgatccgctccgagaacctgaccaacaacgccaagatcatcatcgtgcacctgaacacctccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgctgcatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggactaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 95 - DNA, 1947 bp, synthetic construct
  source 1..1947: mol_type = other DNA
atgggatctctgcaacctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgaacacctctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtcggccggtgtatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggactgataa

SEQ ID NO: 96 - DNA, 1312 bp, synthetic construct
  source 1..1312: mol_type = other DNA
cctgaagtgcaacgacaagaccttcaacggcaagggcccctgcaagaacgtgtccaccgtgcagtgtacccacggcatcagaccagtggtgtctacccagctgctgctgaatggctctctggccgaggaagaagtggtcatcagaagcgacaacttcaccaacaacgccaagaccatcatcgtgcagctgaaagagagcgtcgagatcaactgcacccggcctaacaacaatacccggaagtccatccacatcggccctggcagatggttttacaccaccggcgagattatcggcgacatcagacaggcccactgcaacatcagccgggccaagtggaacgacaccctgaagcagatcgtgatcaagctgagagagcagttcgagaacaagacgatcgtgttcaaccacagctctggcggcgaccccgagattgtgatgcactcctttaactgtggcggcgagttcttctactgcaacagcacccagctgttcaactccacctggaacaacaacacagagggcagcaacaataccgagggcaacaccatcacactgccctgccggatcaagcagatcatcaatatgtggcaagagatcggcaaggctatgtacgcccctcctatccggggccagatcagatgcagcagcaatatcacaggcctgctgctcaccagagatggcggcatcaacgagaacggcaccgagatcttcagacccggcggaggcgacatgcgggacaattggagaagcgagctgtacaagtacaaggtggtcaagatcgagcccctgggcgtcgcccctaccaagtgcaagagaagagtcgtgggccgtagaaggcggcggagagctgttggaattggcgccgtgttcctgggctttctgggagccgctggatctacaatgggcgctgccagcatgaccctgacagtgcaggctagacagctgctgtccggcattgtccagcagcagaacaactgcctgagagcccctgagtgtcaacaaagaatgctgcagctgaccgtgtggggcatcaaacagctgcaggccagagttctggccgtggaaagatacctgggcgaccagcagctcctcggcatctggggatgttctggcaagctgatttgctgcaccgccgtgccttggaatgccagctggtctaacaagagcctggaccggatctggaacaatatgacctggatggaatgggagcgcgagatcgacaactacacctccgagatctacaccctgatcgaggaaagccagaaccagcaagagaaaaacgagcaagagctgctcgagctggattaatga

SEQ ID NO: 97 - DNA, 1944 bp, synthetic construct
  source 1..1944: mol_type = other DNA
atgggctcgctccagccgctcgcgacgctgtacctcctgggcatgctcgtggcgtccgtgctggcggccgagaacctgtgggtgacggtgtactacggcgtgcccgtgtggaaggaggccaacaccacgctgttctgcgccagcgacgccaaggcctacgacaccgaggtgcacaacgtgtgggcgacccacgcctgcgtgccgacggaccccaacccccaggagatcgtgctggagaacgtgaccgagaacttcaacatgtggaagaacaacatggtggagcagatgcacgaggacatcatctcgctgtgggaccagtccctgaagccgtgcgtgaagctgacgcccctgtgcgtgaccctgaactgtagcaccgccaccgtgaacaacagagccgtggacgagatgaagaactgctccttcaacatcaccaccgagatccgcgacaagaagcagaaggtgtacgcgctgttctaccggctggacgtggtgccgatcgacgacaacaacaacaactccagcaactaccgcctgatcaactgcaacaccagcgcctgcacccaggcctgcccgaaggtgtccttcgagcccatccccatccactactgcgcgccggccggcttcgccatcctgaagtgcaacgacaagaagttcaacggcaccggcccctgcaagaacgtgtccaccgtgcagtgcacccacgggatcaagcccgtggtgtccacgcagctgctgctgaacggctccctggccgaggaggagatcatcatccgctccgagaacatcacgaacaacgccaagaccatcatcgtgcagctgaacgagtccgtggagatcaactgcaccaggcccaacaacaacacccgcaagtccatccggatcggccctggccaggcgttctacgccaccggcgacatcatcggcgacatccgccaggcgcactgcaacatctcgggcacgaagtggaacaagaccctgcagcaggtggcgaagaagctgcgcgagcacttcaacaacaagaccatcatcttcaagcccagctccggcggcgacctggagatcacgacccactccttcaactgccgcggcgagttcttctactgcaacacctccggcctgttcaactcgacgtggatcgggaacggcacgaagaacaacaacaacaccaacgacaccatcaccctgccctgccgcatcaagcagatcatcaacatgtggcagggcgtgggccagtgcatgtacgcgccgcccatcgagggcaagatcacctgcaagtccaacatcaccggcctgctcctgacgcgcgacggcggcaacaacaacaccaacgagaccgagatcttcaggccgggcggcggcgacatgcgcgacaactggcgctcggagctgtacaagtacaaggtggtgaagatcgagcccctgggcgtggcgccgacgcgctgcaagagacgcgtggtgggccgcagacgaaggagacgggccgtgggcatcggcgcggtgttcctgggcttcctgggagcagctggttcgacgatgggcgcagcttccatgaccctgacagtgcaggcacgcaacctgctctccggcatcgtccagcagcagtcgaacctgcttcgagcccccgaggcgcagcagcacctcctcaagctgaccgtgtggggcatcaagcagctgcaggcacgcgtgctagccgtggagcgctacctccgcgaccagcagctgctcggaatctggggctgctcgggcaagctgatctgctgcaccaacgtgccgtggaacagctcctggtccaaccgcaacctctcggagatctgggacaacatgacctggctccagtgggacaaggagatctcgaactacacccagatcatctacggcctgctggaggagtcccagaaccagcaggagaagaacgagcaggacctgctggccctggactgataa

SEQ ID NO: 98 - DNA, 1947 bp, synthetic construct; identical to SEQ ID NO: 95
  source 1..1947: mol_type = other DNA

SEQ ID NO: 99 - DNA, 1947 bp, synthetic construct
  source 1..1947: mol_type = other DNA
atgggatctctgcaacctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtcggccggtgtatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggactgataa

SEQ ID NO: 100 - DNA, 2559 bp, synthetic construct
  source 1..2559: mol_type = other DNA
atgagagtgacggggatactgaggaattatccacaatggtggatatggggcatcttaggcttttggatgctaatgacttgtaatggggaaggaaacttgtgggtcacagtctactatggggtaccagtatggaaagaagcaaaaactactctgttttgtgcatcagatgccaaagcgtataagaaagaagtgcataatgtctgggcgacacatgcctgtgtacccacagaccccagcccacaagaactgtttttggaaaatgtaacagaaaattttaacatgtggaaaaatgatatggtagatcagatgcatgaggatataatcagtctatgggatcaaagcctaaagccatgtgtaaagttgaccccactctgtgtcactttaatttgtagtactgctactgttaacaatagagcagttgatgaaatgaaaaattgctccttcaatacaaccacagaaataagagataagaaaaagaaggaatatgcacttttttatagatctgatgtagtaccacttgatgaaacaaacaataccagtgagtatagattaataaattgtaatacctcagccgtaacacaagcctgtccaaaggtcacttttgaaccaattcctatacattattgtgctccagctggttatgcgattctaaagtgcaatgatgagacattcaatggaacaggaccatgcagtaatgtcagcacagtacaatgtacacatggaattaggccagtggtatcaacccaactactgttaaatggtagtctggcagaaaaagagatagtaattagatctgaaaacctgacaaacaatgccaaaataataatagtccatcttcacacccctgtagaaattgtgtgtacaaggcccggccataatacaaggaaaagtgtgagaataggcccaggacaaacattctatgcaacaggagacataataggagatataagacaagcacattgtaacattaatgaaagtaaatggaatgaaactttacaaaaggtaggtatagaattgcaaaaacacttccctaataagacaataaagtataaccaatccgcaggaggagacatggaaattacaacacatagctttaattgtggaggagaatttttctattgcaatacatcaaagctgtttaatagtacatacaatggtacatacataagtacaaacagcacaaacagtacttcatacatcacgcttcaatgcagaataaaacaaattataaacatgtggcagggggtaggaagagcaatgtatgctcctcccattgcaggaaacataacatgtagatcaaatatcacagggctactattgacacgtgatggagggatcaataatgttagcaacgagacagagacattcaggcctgcaggaggagatatgagggataattggagaagtgaattatataaatataaagtagtagaagttcagccattaggaatagcaccaactggtgcaaaaaggagagtggtggagagagaaaaaagagcagcaggactaggagctttgttccttgggttcttgggagcagcaggaagcactatgggcgcagcatcaataacgctgacggtacaggccagacaattgttgtctggtatagtgcaacagcaaagcaatttgctgagggctatagaggcgcaacagcatatgttgcaactcacggtctggggcatcaagcagctccaggcaagagtcctggctctggaaagatacctaaaggatcaacagctcctagggatgtggggctgctctggaaaactcatctgcaccactaatgtgccttggaacactagttggagtaataaatctgaaatggatatttggaataacatgacatggatgcagtgggaaagagaaattagcaattacacagagacaatatacatgttgcttgaagactcgcaacgccagcaggagagaaatgaaaaagatttactagcattggacagttggaacagtctgtggaattggtttaacataacaaactggctgtggtatataaaaatattcataatgatagtagggggcttgataggtttaagaatagtttttgctgtgctatctatagtgaatagagtcaggcagggatactcacctttgtcgttgcagacccttaccccaaacccgagggaacccgacaggctcagaggaatcgaagaagaaggtggagagcaagacagagacagatccattcgattagtgagcggattcttgccaattgtctgggacgacctgcggagcctgtgcctcttcagttaccaccgattgagagactttctattgctggcagcgagagtggtggaacttctgggacacagcagtctcaggggactgcagagggggtgggaagtccttaagtatctgggaagtcttgtgcagtattggggtctggaactaaaaaggagtgctattagtctttttgataccctagcaatagcagtagctgaaggaacagataggattatagaattaatacaaggattttgtagagctatccgcaacatacctacaagaataagacaaggctttgaagcatctttgctataataa

SEQ ID NO: 101 - DNA, 1947 bp, synthetic construct
  source 1..1947: mol_type = other DNA
atgggatctctgcagcctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcacacaggcttgccccaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcaccagacctggccacaacacccggaagtctgtgcggattggacccggccagaccttttatgccaccggcgatatcatcggcgacatcagacaggcccactgtaacatcaacgagagcaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtgggcagatgcatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggactgataa

SEQ ID NO: 102 - DNA, 1947 bp, synthetic construct
  source 1..1947: mol_type = other DNA
atgggatctctgcagcctctggccacactgtacctgctgggaatgctggtggcttctgtgctggccgccgagaatctgtgggtcacagtgtactatggcgtgcccgtgtggaaagaggcctgtaccacactgttctgtgccagcgacgccaaggcctacaagaaaaaggtgcgcaacgtctgggccacacactgctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgacacaggcttgccccaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcaccagacctggccacaacacccggaagtctgtgcggattggacccggccagtggttttatgccaccggcgatatcatcggcgacatcagacaggcccactgtaacatcaacgagagcaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtgggcagagctatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtgcagcagcagagcaattgcctcagagcccctgagtgtcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggactgataa

SEQ ID NO: 103 - protein, 647 aa, synthetic construct
MGSLQPLATLYLLGMLVASVLAAENLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 104 - protein, 642 aa, synthetic construct
MGSLQPLATLYLLGMLVASCLGVEKLWVTVYYGVPVWKEACTTLFCASDAKAYDTKVRNVWATHCCVPTDPNPQEVVLENVTEHFNMWKNNMVEQMQEDIISLWDQSLKPCVKLTPLCVTLNCSTATVNNRAVDEMKNCSFNITTSIRDKVQKEYALFYKLDVVPIDNNNTSYRLISCDTSVITQACPKISFEPIPIHYCAPAGFAILKCNDKTFNGKGPCKNVSTVQCTHGIRPVVSTQLLLNGSLAEEEVVIRSDNFTNNAKTIIVQLKESVEINCTRPNNNTRKSIHIGPGRWFYTTGEIIGDIRQAHCNISRAKWNDTLKQIVIKLREQFENKTIVFNHSSGGDPEIVMHSFNCGGEFFYCNSTQLFNSTWNNNTEGSNNTEGNTITLPCRIKQIINMWQEIGKAMYAPPIRGQIRCSSNITGLLLTRDGGINENGTEIFRPGGGDMRDNWRSELYKYKVVKIEPLGVAPTKCKRRVVGRRRRRRAVGIGAVFLGFLGAAGSTMGAASMTLTVQARQLLSGIVQQQNNCLRAPECQQRMLQLTVWGIKQLQARVLAVERYLGDQQLLGIWGCSGKLICCTAVPWNASWSNKSLDRIWNNMTWMEWEREIDNYTSEIYTLIEESQNQQEKNEQELLELD

SEQ ID NO: 105 - protein, 646 aa, synthetic construct
MGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCSTATVNNRAVDEMKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 106 - protein, 647 aa, synthetic construct; identical to SEQ ID NO: 103

SEQ ID NO: 107 - protein, 647 aa, synthetic construct
MGSLQPLATLYLLGMLVASVLAAENLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 108 - protein, 851 aa, synthetic construct
MRVTGILRNYPQWWIWGILGFWMLMTCNGEGNLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPGHNTRKSVRIGPGQTFYATGDIIGDIRQAHCNINESKWNETLQKVGIELQKHFPNKTIKYNQSAGGDMEITTHSFNCGGEFFYCNTSKLFNSTYNGTYISTNSTNSTSYITLQCRIKQIINMWQGVGRAMYAPPIAGNITCRSNITGLLLTRDGGINNVSNETETFRPAGGDMRDNWRSELYKYKVVEVQPLGIAPTGAKRRVVEREKRAAGLGALFLGFLGAAGSTMGAASITLTVQARQLLSGIVQQQSNLLRAIEAQQHMLQLTVWGIKQLQARVLALERYLKDQQLLGMWGCSGKLICTTNVPWNTSWSNKSEMDIWNNMTWMQWEREISNYTETIYMLLEDSQRQQERNEKDLLALDSWNSLWNWFNITNWLWYIKIFIMIVGGLIGLRIVFAVLSIVNRVRQGYSPLSLQTLTPNPREPDRLRGIEEEGGEQDRDRSIRLVSGFLPIVWDDLRSLCLFSYHRLRDFLLLAARVVELLGHSSLRGLQRGWEVLKYLGSLVQYWGLELKRSAISLFDTLAIAVAEGTDRIIELIQGFCRAIRNIPTRIRQGFEASLL

SEQ ID NO: 109 - protein, 647 aa, synthetic construct
MGSLQPLATLYLLGMLVASVLAAENLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPGHNTRKSVRIGPGQTFYATGDIIGDIRQAHCNINESKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 110 - protein, 647 aa, synthetic construct
MGSLQPLATLYLLGMLVASVLAAENLWVTVYYGVPVWKEACTTLFCASDAKAYKKKVRNVWATHCCVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPGHNTRKSVRIGPGQWFYATGDIIGDIRQAHCNINESKWNETLQKVGIELQKHFPNKTIKYNQSAGGDMEITTHSFNCGGEFFYCNTSKLFNSTYNGTYISTNSTNSTSYITLQCRIKQIINMWQGVGRAMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNCLRAPECQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 111 - protein, 6 aa, synthetic construct
LPSTGG

SEQ ID NO: 112 - protein, 5 aa, synthetic construct
LPSTG

SEQ ID NO: 113 - protein, 655 aa, synthetic construct
MPMGSLQPLATLYLLGMLVASVLAAENLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDLPSTGG

SEQ ID NO: 114 - DNA, 1989 bp, synthetic construct
  source 1..1989: mol_type = other DNA
gtcgacgccaccatgcccatgggatctctgcaacctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtcggccggtgtatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggacctgcctagcaccggaggatgataaggatcc

SEQ ID NO: 115 - DNA, 2445 bp, synthetic construct
  source 1..2445: mol_type = other DNA
gtcgacgccaccatgcctatgggatctctgcagcctctggccacactgtacctgctgggaatgctggtggctagcgtgctggccaagggaaagctgtgggtcacagtgtactacggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcagacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtggaaatccagccactgggaatcgccccaacaggctgcaagagaagagtggttgaaggcggcggaggatctggcggaggcggatctgcagttggaatcggagctgtgttcctgggctttctgggagccgccggatctacaatgggagctgccagcatgaccctgaccgtgcaggctagaaatctgctgagcggcaaccccgactggctgcctgatatgacagtgtggggcatcaagcagctgcaggccagagtgctggccgtggaaagatacctgagagatcagcagctcctcggaatctggggctgtagcggcaagctgatctgctgcaccaacgtgccctggaacagctcctggtccaacagaaacctgtccgagatctgggataacatgacctggctgcagtgggacaaagagatctccaactacacccagatcatctacggcctgctggaagagagccagaaccagcaagagaaaaacgagcaggacctgctggccctggatggcggaggaagcggagatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagatcgagctgatcggcaacgagaaccacggcctgtatctggccgaccagtacgtgaagggcattgccaagagccggaagtcctgatgaggatcc

SEQ ID NO: 116 - DNA, 2472 bp, synthetic construct
  source 1..2472: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggcctgcaccacactgttttgcgcctccgatgccagagcctacgagaagaaagtgcacaacgtgtgggccacacactgctgcgtgccaaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcacacaggcttgccccaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcacctgaataccagcgtcgagatcgtgtgcacccggcctaacaacaacacccggaagtctgtgcggatcggccctggccagtggttttatgccaccggcgatatcatcggcgacatcagacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaattgtctgagagcccctgagtgccagcagcatctgctgaaactgaccgtgtggggcatcaagcagctgcaggcaagagtgctggcagtggaaagatacctgcgggaccagcagctcctcggaatctggggatgtagcggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcgacattatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaggtgctgttcaaggacatcctggacaagattgagctgatcggcaacgagaaccacggcctgtatctggccgaccagtacgtgaagggaatcgccaagagcagaaagagctgatgaggatcc

SEQ ID NO: 117 - DNA, 2472 bp, synthetic construct
  source 1..2472: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtggccaacgtttgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgccgtgacacaggcttgccccaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcacctgaataccagcgtcgagatcgtgtgcacccggcctaacaacaacacccggaagtctgtgcggatcggccctggccagacattctatgccaccggcgatatcatcggcgacatcagacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtgggcagagctatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggccagagtgctgacagtggaaagatacgccagggaccagcagctcctcggaatctggggatgtagcggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggctctggatggcggaggaagcggagatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 118 - DNA, 2472 bp, synthetic construct
  source 1..2472: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacatctgggccacacacgcctgcgtgccaaccgatccatctcctcaagaactggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgccgtgacacaagtgtgccccaagctgaccttcgagcccattcctatccactactgtgcccctgccggctacgccatcctgaagtgcaacaacgagacattcaacggcacaggcccctgcagcaatgtgtccaccgtgcagtgtacccacggcatcagacctgtgctgagcacacagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcagacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtgggcagagctatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagcagctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcgacattatcaagctgctgaacgagcaagtgaacaaagaaatgaacagctccaacctgtacatgagcatgtccagctggtgctacacccacagtcttgatggcgccggactgttcctgtttgaccacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 119 - DNA, 2472 bp, synthetic construct
  source 1..2472: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacatctgggccacacacgcctgcgtgccaaccgatccatctcctcaagaactggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacaagcgccgtgacaatggtctgccccaagctgaccttcgagcccattcctatccactactgtgcccctgccggctacgccatcctgaagtgcaacaacgagacattcaacggcacaggcccctgcagcaatgtgtccaccgtgcagtgtacccacggcatcagacctgtgctgagcacacagctgctgctgaatggaagcctggccgagaaagaaatcgtgatcagaagcgagaacctgaccaacaacgccaagatcatcatcgtccacctgaacacctccgtggaaatcgtgtgcacccggcctctgaatctgaccagaaagagcgtgcggatcggccctggccagaccttttatgccatgggcgacatcatcggcgacatccggcaggcccactgcaacatctctgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagatgatcattaacatgtggcaaggcgtcggcagggctatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagcagctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcgatatcatcaagctgctgaacgagcaagtgaacaaagaaatgaacagctccaacctgtacatgagcatgtccagctggtgctacacccacagtcttgatggcgccggactgttcctgtttgaccacgccgccgaggaatacgagcacgccaagaagctgatcattttcctgaacgagaacaacgtgccagtgcagctgaccagcattagcgccccagagcacaagttcgagggcctgacacagatctttcagaaggcctacgaacacgagcagcacatcagcgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 120 - DNA, 2478 bp, synthetic construct
  source 1..2478: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtggtgccagtctctgaagccctgcgtgaagctgacccctctgtgtgtgaccctgatctgctctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgccgtgacacaggcttgccccaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcagacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggctgcggcgtgggcagagctatgtatgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgttgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcgacattatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 121 - DNA, 2472 bp, synthetic construct
  source 1..2472: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcagacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcgacattatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 122 - protein, 807 aa, synthetic construct
MPMGSLQPLATLYLLGMLVASVLAKGKLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIRQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVEIQPLGIAPTGCKRRVVEGGGGSGGGGSAVGIGAVFLGFLGAAGSTMGAASMTLTVQARNLLSGNPDWLPDMTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 123 - protein, 816 aa, synthetic construct
MPMGSLQPLATLYLLGMLVASVLAAENLWVTVYYGVPVWKEACTTLFCASDARAYEKKVHNVWATHCCVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQWFYATGDIIGDIRQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNCLRAPECQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 124 - protein, 816 aa, synthetic construct
MPMGSLQPLATLYLLGMLVASVLAAENLWVTVYYGVPVWKEAKTTLFCASDARAYEKEVANVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIRQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLTVERYARDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 125 - protein, 816 aa, synthetic construct
MPMGSLQPLATLYLLGMLVASVLAAENLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIRQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 126 - protein, 816 aa, synthetic construct
MPMGSLQPLATLYLLGMLVASVLAAENLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTMVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPLNLTRKSVRIGPGQTFYAMGDIIGDIRQAHCNISEEKWNDTLQKVGIELQKHFPNKTIKYNQSAGGDMEITTHSFNCGGEFFYCNTSNLFNGTYNGTYISTNSSANSTSTITLQCRIKMIINMWQGVGRAMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 127 - protein, 818 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWCQSLKPCVKLTPLCVTLICSDATVKTGTVEEMKNCSFNTTTEIRDKKKKEYALFYKPDIVPLSETNNTSEYRLINCNTSAVTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIRQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGCGVGRAMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 128 - protein, 816 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIRQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 129 - DNA, 2532 bp, synthetic construct
  source 1..2532: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcggaggcggttctggcggcggaggaagcggaggcggaggctcaggtggtggcggatctggagatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 130 - DNA, 2517 bp, synthetic construct
  source 1..2517: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcggaggcggttctggcggcggaggaagcggaggcggaggctcaggtgatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 131 - DNA, 2502 bp, synthetic construct
  source 1..2502: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcggaggcggttctggcggcggaggaagcggagatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 132 - DNA, 2487 bp, synthetic construct
  source 1..2487: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcggaggcggttctggcgatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 133 - DNA, 2541 bp, synthetic construct
  source 1..2541: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcggaggcggttctggcggcggaggaagcggaggcggaggctcaggtggtggcggatctggactgagcaaggatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 134 - DNA, 2526 bp, synthetic construct
  source 1..2526: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcggaggcggttctggcggcggaggaagcggaggcggaggctcaggtctgagcaaggatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 135 - DNA, 2511 bp, synthetic construct
  source 1..2511: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcggaggcggttctggcggcggaggaagcggactgagcaaggatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 136 - DNA, 2496 bp, synthetic construct
  source 1..2496: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcggaggcggttctggcctgagcaaggatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 137 - DNA, 2481 bp, synthetic construct
  source 1..2481: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcctgagcaaggatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 138 - protein, 836 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGGGGSGGGGSGGGGSGGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 139 - protein, 831 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGGGGSGGGGSGGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 140 - protein, 826 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGGGGSGGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 141 - protein, 821 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 142 - protein, 839 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGGGGSGGGGSGGGGSGGGGSGLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 143 - protein, 834 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGGGGSGGGGSGGGGSGLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 144 - protein, 829 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGGGGSGGGGSGLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 145 - protein, 824 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGGGGSGLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 146 - protein, 819 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 147 - DNA, 2223 bp, synthetic construct
  source 1..2223: mol_type = other DNA
gtcgacgccaccatgcctatgggatctctgcagcctctggccacactgtacctgctgggaatgctggtggcttctgtgctggccgagggcaatctgtgggtcacagtgtactatggcgtgcccgtgtggaaagaggccaagaccacactgttctgtgccagcgacgccaaggcctacaagaaagaggtggccaacgtctgggccacacacgcctgtgttcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgacacaggcttgccccaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtgggcagagctatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtggaagtgcagcccctgggaatcgctccaacaggcgctaagagaagagtggtggaacgcgagaaaagagccgctggactgggagccctgtttctgggttttctgggagccgccggaagcacaatgggagctgcctctatcacactgaccgtgcaggctagacagctgctgagcggaattgtgcagcagcagagcaacctgctgagagccattgaagcccagcagcacatgctgcagctgaccgtgtggggaatcaaacagctgcaggccagagtgctgaccctggaaagatacgccaaggaccagcagctcctcggcatgtggggatgttctggcaagctgatctgcaccaccaacgtgccctggaacacctcctggtccaacaagagcgagatggacatctggaacaacatgacctggatgcagtgggagagagagatcagcaactacaccgagacaatctacatgctgctcgaggacagccagcggcagcaagagagaaacgagaaggatctgctggccctggactcctggaatagcctgtggaactggttcaatatcaccaactggctgtggtacatcaagatcttcatcatgatcgtcggcggcctgatcggcctgagaatcgtgtttgccgtgctgagcatcgtgaacagagtgcggcagggaatcagcccactgagcctgcaaaccctgacacctaatcctagagagcccgaccggctgagaggcatcgaagaagaaggcggcgagcaggacagagacagatccatcagactggtgtcctgataaggatcc

SEQ ID NO: 148 - DNA, 2109 bp, synthetic construct
  source 1..2109: mol_type = other DNA
gtcgacgccaccatgcctatgggatctctgcagcctctggccacactgtacctgctgggaatgctggtggcttctgtgctggccgagggcaatctgtgggtcacagtgtactatggcgtgcccgtgtggaaagaggccaagaccacactgttctgtgccagcgacgccaaggcctacaagaaagaggtggccaacgtctgggccacacacgcctgtgttcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgacacaggcttgccccaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtgggcagagctatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtggaagtgcagcccctgggaatcgctccaacaggcgctaagagaagagtggtggaacgcgagaaaagagccgctggactgggagccctgtttctgggttttctgggagccgccggaagcacaatgggagctgcctctatcacactgaccgtgcaggctagacagctgctgagcggaattgtgcagcagcagagcaacctgctgagagccattgaagcccagcagcacatgctgcagctgaccgtgtggggaatcaaacagctgcaggccagagtgctgaccctggaaagatacgccaaggaccagcagctcctcggcatgtggggatgttctggcaagctgatctgcaccaccaacgtgccctggaacacctcctggtccaacaagagcgagatggacatctggaacaacatgacctggatgcagtgggagagagagatcagcaactacaccgagacaatctacatgctgctcgaggacagccagcggcagcaagagagaaacgagaaggatctgctggccctggactcctggaatagcctgtggaactggttcaatatcaccaactggctgtggtacatcaagatcttcatcatgatcgtcggcggcctgatcggcctgagaatcgtgtttgccgtgctgagcatcgtgaacagagtgcggcagggatactgataaggatcc

SEQ ID NO: 149 - DNA, 2475 bp, synthetic construct
  source 1..2475: mol_type = other DNA
gtcgacgccaccatgggatctctgcaacctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtggtgccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgaacacctctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggctgcggcgtcggccgggctatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggacggcggaggatctggcgacattatcaagctgctgaacgagcaagtgaacaaagagatgaacagctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgataaggatcc

SEQ ID NO: 150 - DNA, 2469 bp, synthetic construct
  source 1..2469: mol_type = other DNA
gtcgacgccaccatgggatctctgcaacctctggccacactgtacctgctgggaatgctggtggcttctgtgctggccgccgagaatctgtgggtcacagtgtactatggcgtgcccgtgtggaaagaggcctgcaccacactgttctgtgccagcgacgccaaggcctacaagaaaaaggtgcggaacgtctgggccacacactgctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgaacacctctgtggaaatcgtgtgcacccggcctaacaacaacacccggaagtctgtgcggatcggccctggccagtggttctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtcggccggtgtatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaattgcctcagagcccctgagtgccagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggacggcggaggatctggcgacattatcaagctgctgaacgagcaagtgaacaaagagatgaacagctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgataaggatcc

SEQ ID NO: 151 - DNA, 2469 bp, synthetic construct
  source 1..2469: mol_type = other DNA
gtcgacgccaccatgggatctctgcaacctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacatctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgactatggtgtgtcctaaactgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtgctgtctacccagctgctgctgaatggaagcctggccgagaaagaaatcgtgatcagaagcgagaacctgaccaacaacgccaagatcatcattgtgcatctgaacacctctgtggaaatcgtgtgcacccggcctctgaacctgacccggaagtctgtgcggatcggccctggccagacattctatgccatgggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagatgatcatcaatatgtggcaaggcgtcggccgggctatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggacggcggaggatctggcgacattatcaagctgctgaacgagcaagtgaacaaagagatgaacagctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgataaggatcc

SEQ ID NO: 152 - DNA, 2469 bp, synthetic construct
  source 1..2469: mol_type = other DNA
gtcgacgccaccatgggatctctgcaacctctggccacactgtacctgctgggaatgctggtggcttctgtgctggccgccgagaatctgtgggtcacagtgtactatggcgtgcccgtgtggaaagaggcctgcaccacactgttctgtgccagcgacgccaaggcctacaagaaaaaggtgcggaacgtctgggccacacactgctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgaacacctctgtggaaatcgtgtgcacccggcctaacaacaacacccggaagtctgtgcggatcggccctggccagtggttctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtcggccgggctatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaattgcctcagagcccctgagtgccagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggacggcggaggatctggcgacattatcaagctgctgaacgagcaagtgaacaaagagatgaacagctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgataaggatcc

SEQ ID NO: 153 - DNA, 2469 bp, synthetic construct
  source 1..2469: mol_type = other DNA
gtcgacgccaccatgggatctctgcaacctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgaacacctctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtcggccggtgtatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggacggcggaggatctggcgacattatcaagctgctgaacgagcaagtgaacaaagagatgaacagctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgataaggatcc

SEQ ID NO: 154 - DNA, 2475 bp, synthetic construct
  source 1..2475: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacatctgggccacacacgcctgcgtgccaaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgacacaagtgtgccccaagctgaccttcgagcccattcctatccactactgtgcccctgccggctacgccatcctgaagtgcaacaacgagacattcaacggcacaggcccctgcagcaatgtgtccaccgtgcagtgtacccacggcatcagacctgtgctgagcacacagctgctgctgaatggaagcctggccgagaaagaaatcgtgatcagaagcgagaacctgaccaacaacgccaagatcatcatcgtgcacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtgggcagagctatgtacgcccctcctatcgccggcaacatcacctgtcggagcaatatcacaggcctgctgctcaccagagatggcggcatcaacaacgtgtccaacgaaaccgaaaccttccggcctgccggcggagacatgagagacaattggagaagcgagctgtacaagtacaaggtggtcaagatcgagcccctgggcgtcgcaccaacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaacagcagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggatggcggaggatctggcgacattatcaagctgctgaacgagcaagtgaacaaagaaatgaacagctccaacctgtacatgagcatgtccagctggtgttacacccacagccttgatggcgccggactgttcctgtttgatcacgccgccgaggaatacgagcacgccaagaagctgatcatcttcctgaacgagaacaatgtgcccgtgcagctgaccagcatctctgcccctgagcacaagttcgagggcctgacacagatctttcagaaggcctacgaacacgagcagcacatctccgagtccatcaacaatatcgtggaccacgccattaagagcaaggatcacgccaccttcaactttctccagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 155 - DNA, 2448 bp, synthetic construct
  source 1..2448: mol_type = other DNA
gtcgacgccaccatgcctatgggatctctgcagcctctggccacactgtacctgctgggaatgctggtggcttctgtgctggccgagggcaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtgcacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtcggccggtgtatgtacgcccctcctatcgccggcaacatcacctgtcggagcaatatcacaggcctgctgctcaccagagatggcggcatcaacaacgtgtccaacgaaaccgaaaccttccggcctgccggcggagacatgagagacaattggagaagcgagctgtacaagtacaaggtggtggaagtgcagcccctgggaatcgctcctaccggctgcaagagaagagtggttgaaggcggcggaggatctggcggaggcggatctgctgttggaatcggagctgtgttcctgggctttctgggagccgccggatctacaatgggagctgccagcatgaccctgaccgtgcaggctagaaatctgctgagcggcaaccccgactggctgcctgatatgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggatggcggaggaagcggagatatcatcaagctgctgaacgagcaagtgaacaaagaaatgaacagcagcaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcatctctgcccctgagcacaagttcgagggcctgacacagatctttcagaaggcctacgaacacgagcagcacatctccgagtccatcaacaatatcgtggaccacgccattaagagcaaggatcacgccaccttcaactttctccagtggtacgtggccgaacagcacgaggaagaagtgctgttcaaggacatcctggacaagatcgagctgatcggcaacgagaaccacggcctgtatctggccgaccagtacgtgaagggaatcgccaagagccggaagtcctgatgaggatcc

SEQ ID NO: 156 - DNA, 2475 bp, synthetic construct
  source 1..2475: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtggccaacgtctgggccacacacgcctgtgttcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgacacaggcttgccccaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtgcacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtgggcagagctatgtacgcccctcctatcgccggcaacatcacctgtcggagcaatatcacaggcctgctgctcaccagagatggcggcatcaacaacgtgtccaacgaaaccgaaaccttccggcctgccggcggagacatgagagacaattggagaagcgagctgtacaagtacaaggtggtcaagatcgagcccctgggcgtcgcaccaacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctgaccgtggaaagatacgccagagatcagcagctcctcggaatctggggctgtagcggcaagctgatctgctgcaccaacgtgccctggaactccagctggtccaaccggaatctgagcgagatctgggataacatgacctggctgcagtgggacaaagagatctccaactacacccagatcatctacggcctgctggaagagagccagaaccagcaagagaaaaacgagcaggacctgctggccctggatggcggaggatctggcgacattatcaagctgctgaacgagcaagtgaacaaagaaatgaacagcagcaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcatctctgcccctgagcacaagttcgagggcctgacacagatctttcagaaggcctacgaacacgagcagcacatctccgagtccatcaacaatatcgtggaccacgccattaagagcaaggatcacgccaccttcaactttctccagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 157 - protein, 733 aa, synthetic construct
MPMGSLQPLATLYLLGMLVASVLAEGNLWVTVYYGVPVWKEAKTTLFCASDAKAYKKEVANVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRAMYAPPIAGNITCRSNITGLLLTRDGGINNVSNETETFRPAGGDMRDNWRSELYKYKVVEVQPLGIAPTGAKRRVVEREKRAAGLGALFLGFLGAAGSTMGAASITLTVQARQLLSGIVQQQSNLLRAIEAQQHMLQLTVWGIKQLQARVLTLERYAKDQQLLGMWGCSGKLICTTNVPWNTSWSNKSEMDIWNNMTWMQWEREISNYTETIYMLLEDSQRQQERNEKDLLALDSWNSLWNWFNITNWLWYIKIFIMIVGGLIGLRIVFAVLSIVNRVRQGISPLSLQTLTPNPREPDRLRGIEEEGGEQDRDRSIRLVS

SEQ ID NO: 158 - protein, 695 aa, synthetic construct
MPMGSLQPLATLYLLGMLVASVLAEGNLWVTVYYGVPVWKEAKTTLFCASDAKAYKKEVANVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRAMYAPPIAGNITCRSNITGLLLTRDGGINNVSNETETFRPAGGDMRDNWRSELYKYKVVEVQPLGIAPTGAKRRVVEREKRAAGLGALFLGFLGAAGSTMGAASITLTVQARQLLSGIVQQQSNLLRAIEAQQHMLQLTVWGIKQLQARVLTLERYAKDQQLLGMWGCSGKLICTTNVPWNTSWSNKSEMDIWNNMTWMQWEREISNYTETIYMLLEDSQRQQERNEKDLLALDSWNSLWNWFNITNWLWYIKIFIMIVGGLIGLRIVFAVLSIVNRVRQGY

SEQ ID NO: 159 - protein, 817 aa, synthetic construct
MGSLQPLATLYLLGMLVASVLAAENLWVTVYYGVPVWKEAKTTLFCASDAKAYKKEVHNVWATHACVPTDPSPQELFLENVTENFNMWKNDMVDQMHEDIISLWCQSLKPCVKLTPLCVTLICSTATVNNRAVDEMKNCSFNTTTEIRDKKKKEYALFYRSDVVPLDETNNTSEYRLINCNTSAVTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGCGVGRAMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 160 - protein, 815 aa, synthetic construct
MGSLQPLATLYLLGMLVASVLAAENLWVTVYYGVPVWKEACTTLFCASDAKAYKKKVRNVWATHCCVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQWFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNCLRAPECQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 161 - protein, 815 aa, synthetic construct
MGSLQPLATLYLLGMLVASVLAAENLWVTVYYGVPVWKEAKTTLFCASDAKAYKKEVHNIWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTMVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPLNLTRKSVRIGPGQTFYAMGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKMIINMWQGVGRAMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 162 - protein, 815 aa, synthetic construct
MGSLQPLATLYLLGMLVASVLAAENLWVTVYYGVPVWKEACTTLFCASDAKAYKKKVRNVWATHCCVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQWFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRAMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNCLRAPECQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 163 - protein, 815 aa, synthetic construct
MGSLQPLATLYLLGMLVASVLAAENLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 164 - protein, 817 aa, synthetic construct
MPMGSLQPLATLYLLGMLVASVLAAENLWVTVYYGVPVWKEAKTTLFCASDAKAYKKEVHNIWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRAMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 165 - protein, 808 aa, synthetic construct
MPMGSLQPLATLYLLGMLVASVLAEGNLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVEVQPLGIAPTGCKRRVVEGGGGSGGGGSAVGIGAVFLGFLGAAGSTMGAASMTLTVQARNLLSGNPDWLPDMTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 166 - protein, 817 aa, synthetic construct
MPMGSLQPLATLYLLGMLVASVLAAENLWVTVYYGVPVWKEAKTTLFCASDAKAYKKEVANVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRAMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLTVERYARDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 167 - protein, 6 aa, synthetic construct
GGGGGG

SEQ ID NO: 168 - protein, 5 aa, synthetic construct
LPSTG

SEQ ID NO: 169 - DNA, 2454 bp, synthetic construct
  source 1..2454: mol_type = other DNA
atgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaaacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggattctggcggagacattatcaagctgctgaacgagcaagtgaacaaagaaatgcagtcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtccggctcctgatga

SEQ ID NO: 170 - DNA, 1968 bp, synthetic construct
  source 1..1968: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggattgatgaggatcc

SEQ ID NO: 171 - DNA, 1968 bp, synthetic construct
  source 1..1968: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctaacgccaccgtgaaaaacggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggattgatgaggatcc

SEQ ID NO: 172 - DNA, 2472 bp, synthetic construct
  source 1..2472: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctaacgccaccgtgaaaaacggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaaacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggattctggcggagacattatcaagctgctgaacgagcaagtgaacaaagaaatgcagtcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtccggctcctgatgaggatcc

SEQ ID NO: 173 - protein, 792 aa, synthetic construct
AENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 174 - protein, 624 aa, synthetic construct
AENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 175 - protein, 624 aa, synthetic construct
AENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSNATVKNGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 176 - protein, 792 aa, synthetic construct
AENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSNATVKNGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 177 - DNA, 1989 bp, synthetic construct
  source 1..1989: mol_type = other DNA
gtcgacgccaccatgcctatgggatctctgcaacctctggccacactgtacctgctgggaatgctggtggcttctgtgctggccgccgagaatctgtgggtcacagtgtactatggcgtgcccgtgtgggaagatgccgacacacctctgttttgtgccagcgacgccaaggcctacagcaccgagtctcacaatgtgtgggccactcacgcctgcgtgccaaccgatccatctcctcaagagatcagcctggacaacgtgaccgagaacttcaacatgtggaagaacaacatggtcgagcagatgcacgaggacatcatcagcctgtgggacgagagcctgaagccttgcgtgaagctgacccctctgtgcgtgaccctgaactgcaccaacgtgaacaacagcagcgccaccaacaactccatggtggacgatagagagggcctgaagaactgcagcttcaacatcaccaccgagctgcgggacaagaagaagcaagagcacgccctgttctaccggctggacatcgtgcccatcaacggcaacagcaactccaacagctccgtgggcgactacagactgatcaactgcaacgtgtccacatgcaagcaggcctgtcctaagatgagcttcgaccccattcctatccactactgtgcccctgccggcttcgccatcctgaagtgcagagacaagaagttcaacggcaccggcagctgcaagaatgtgtccaccgtgcagtgtacccacggcatcaagcctgtgatcagcacccagctgctgctgaatggctctgtggccgaagaggaaatcatgatccgcagcgagaactttaccaacaacgccaagaacatcatcgtccagttcaacaagaccatcgacatcatgtgcacccggcctaacaacaacacccggaagtccatcagcctcggacctggacaggccatctatgccaccggcgacatcatcggcaatatcagacaggcccactgcaacatctctggcgccgactggggcaacatgatcagaaatgtgtccgagaagctgaaagagatcttcaacaaaaccaccatcaccttcaaggcctccgctggcggcgacctggaaatcaccacacacagcttcaattgcaggggcgagttcttctactgcgacaccagcgacctgttcaactccagcagattcaacaatagcagcaacgacacgaacgacaccatcacactgccctgcaagatcaagcagatcgtgcggatgtggcagcgcgtgggccagtgtatgtatgcccctcctatcgccggcaacatcacatgcagaagcaatatcaccggcctgctgctcaccagagatggcggcggaaacaacaccaacgagacagaaaccttccggcctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggctgtggaaagatacctgagggaccagcagctcctcggaatctggggctgttctggcaagctgatctgctgcaccaatgtgccctggaacagctcctggtccaaccggaatctgagcgagatctgggacaatatgacctggctgcagtgggacaaagagattagcaactacacccagatcatctacggactgctggaagagagccagaaccagcaagagaaaaacgagcaggacctgctggccctggactgataaggatcc

SEQ ID NO: 178 - DNA, 1989 bp, synthetic construct
  source 1..1989: mol_type = other DNA
gtcgacgccaccatgcctatgggatctctgcagcctctggccacactgtacctgctgggaatgctggtggctagcgtgctggcctccaacaatctgtgggtcaccgtgtactacggcgtgcccgtttgggaagatgccgacacacctctgttctgtgcctccgatgccaaggcctacagcaccgagtctcacaacgtgtgggccactcatgcctgcgtgccaaccgatccatctcctcaagagatcagcctggacaacgtgaccgagaacttcaacatgtggaagaacaacatggtcgagcagatgcacgaggacatcatcagcctgtgggacgagagcctgaagccttgcgtgaagctgacccctctgtgcgtgaccctgaactgcaccaacgtgaacaacagcagcgccaccaacaactccatggtggacgatagagagggcctgaagaactgcagcttcaacatcaccaccgagctgcgggacaagaagaagcaagagcacgccctgttctaccggctggacatcgtgcccatcaacggcaacagcaactccaacagctccgtgggcgactacagactgatcaactgcaacgtgtccaccatcaagcaggcctgtcctaagatgagcttcgaccccattcctatccactactgtgcccctgccggcttcgccatcctgaagtgcagagacaagaagttcaacggcaccggcagctgcaagaatgtgtccaccgtgcagtgtacccacggcatcaagcctgtgatcagcacccagctgctgctgaatggctctgtggccgaagaggaaatcatgatccgcagcgagaactttaccaacaacgccaagaacatcatcgtccagttcaacaagaccatcgacatcatgtgcacccggcctaacaacaacacccggaagtccatcagcctcggacctggacaggccatctatgccaccggcgacatcatcggcaatatcagacaggcccactgcaacatctctggcgccgactggggcaacatgatcagaaatgtgtccgagaagctgaaagagatcttcaacaaaaccaccatcaccttcaaggcctccgctggcggcgacctggaaatcaccacacacagcttcaattgcaggggcgagttcttctactgcgacaccagcgacctgttcaactccagcagattcaacaatagcagcaacgacacgaacgacaccatcacactgccctgcaagatcaagcagatcgtgcggatgtggcagcgcgttggccaggctatgtatgcccctcctatcgccggcaacatcacatgcagaagcaatatcaccggcctgctgctcaccagagatggcggcggaaacaacaccaacgagacagaaaccttccggcctgccggcggagacatgagagacaattggagaagcgagctgtacaagtacaaggtggtcaagatcaaccctctgggcgtcgcccctaccagatgtagaagaagagtcgtcggcagacggcggaggcggagagctattggaatgggagctgtgctgctgggctttctgggaacagccggctctacaatgggagccgcctctatcaccctgaccgtgcaagttcgacagctgctgtctggcatcgtgcagcagcagagcaacctgctgagagcccctgaagctcagcagcatctcctgcagctgacagtgtggggaatcaagcagctccaggccagaatcctggccgtcgagagatacctgaaggaccagcagctcctcggaatctggggctgttctggcaagctgatctgctgcaccaatgtgccctggaacgccagctggtccaacagaagctacaacgacatctgggagaacctgacctgggtcgagtgggagagagagatctccaactacacccagcagatctacagcctgctggaagagagccagaaccagcaagagaagaacgagcaggacctgctggccctggactgataaggatcc

SEQ ID NO: 179 - DNA, 1989 bp, synthetic construct
  source 1..1989: mol_type = other DNA
gtcgacgccaccatgcctatgggatctctgcagcctctggccacactgtacctgctgggaatgctggtggctagcgtgctggcctccaacaatctgtgggtcaccgtgtactacggcgtgcccgtttgggaagatgccgacacacctctgttctgtgcctccgatgccaaggcctacagcaccgagtctcacaacgtgtgggccactcatgcctgcgtgccaaccgatccatctcctcaagagatcagcctggacaacgtgaccgagaacttcaacatgtggaagaacaacatggtcgagcagatgcacgaggacatcatcagcctgtgggacgagagcctgaagccttgcgtgaagctgacccctctgtgcgtgaccctgaactgcaccaacgtgaacaacagcagcgccaccaacaactccatggtggacgatagagagggcctgaagaactgcagcttcaacatcaccaccgagctgcgggacaagaagaagcaagagcacgccctgttctaccggctggacatcgtgcccatcaacggcaacagcaactccaacagctccgtgggcgactacagactgatcaactgcaacgtgtccacctgcaagcaggcctgtcctaagatgagcttcgaccccattcctatccactactgtgcccctgccggcttcgccatcctgaagtgcagagacaagaagttcaacggcaccggcagctgcaagaatgtgtccaccgtgcagtgtacccacggcatcaagcctgtgatcagcacccagctgctgctgaatggctctgtggccgaagaggaaatcatgatccgcagcgagaactttaccaacaacgccaagaacatcatcgtccagttcaacaagaccatcgacatcatgtgcacccggcctaacaacaacacccggaagtccatcagcctcggacctggacaggccatctatgccaccggcgacatcatcggcaatatcagacaggcccactgcaacatctctggcgccgactggggcaacatgatcagaaatgtgtccgagaagctgaaagagatcttcaacaaaaccaccatcaccttcaaggcctccgctggcggcgacctggaaatcaccacacacagcttcaattgcaggggcgagttcttctactgcgacaccagcgacctgttcaactccagcagattcaacaatagcagcaacgacacgaacgacaccatcacactgccctgcaagatcaagcagatcgtgcggatgtggcagcgcgttggccagtgcatgtatgcccctcctatcgccggcaacatcacatgcagaagcaatatcaccggcctgctgctcaccagagatggcggcggaaacaacaccaacgagacagaaaccttccggcctgccggcggagacatgagagacaattggagaagcgagctgtacaagtacaaggtggtcaagatcaaccctctgggcgtcgcccctaccagatgtagaagaagagtcgtcggcagacggcggaggcggagagctattggaatgggagctgtgctgctgggctttctgggaacagccggctctacaatgggagccgcctctatcaccctgaccgtgcaagttcgacagctgctgtctggcatcgtgcagcagcagagcaacctgctgagagcccctgaagctcagcagcatctcctgcagctgacagtgtggggaatcaagcagctccaggccagaatcctggccgtcgagagatacctgaaggaccagcagctcctcggaatctggggctgttctggcaagctgatctgctgcaccaatgtgccctggaacgccagctggtccaacagaagctacaacgacatctgggagaacctgacctgggtcgagtgggagagagagatctccaactacacccagcagatctacagcctgctggaagagagccagaaccagcaagagaagaacgagcaggacctgctggccctggactgataaggatcc

SEQ ID NO: 180 - DNA, 1950 bp, synthetic construct
  source 1..1950: mol_type = other DNA
gtcgacgccaccatgcctatgggatctctgcagcctctggccacactgtacctgctgggaatgctggtggctagcgtgctggcctccaacaatctgtgggtcaccgtgtactacggcgtgcccgtttgggaagatgccgacacacctctgttctgtgcctccgatgccaaggcctacagcaccgagtctcacaacgtgtgggccactcatgcctgcgtgccaaccgatccatctcctcaagagatcagcctggacaacgtgaccgagaacttcaacatgtggaagaacaacatggtcgagcagatgcacgaggacatcatcagcctgtgggacgagagcctgaagccttgcgtgaagctgacccctctgtgcgtgaccctgaactgcaccaacgtgaacaacagcagcgccaccaacaactccatggtggacgatagagagggcctgaagaactgcagcttcaacatcaccaccgagctgcgggacaagaagaagcaagagcacgccctgttctaccggctggacatcgtgcccatcaacggcaacagcaactccaacagctccgtgggcgactacagactgatcaactgcaacgtgtccacatgcaagcaggcctgtcctaagatgagcttcgaccccattcctatccactactgtgcccctgccggcttcgccatcctgaagtgcagagacaagaagttcaacggcaccggcagctgcaagaatgtgtccaccgtgcagtgtacccacggcatcaagcctgtgatcagcacccagctgctgctgaatggctctgtggccgaagaggaaatcatgatccgcagcgagaactttaccaacaacgccaagaacatcatcgtccagttcaacaagaccatcgacatcatgtgcacccggcctaacaacaacacccggaagtccatcagcctcggacctggacaggccatctatgccaccggcgacatcatcggcaatatcagacaggcccactgcaacatctctggcgccgactggggcaacatgatcagaaatgtgtccgagaagctgaaagagatcttcaacaaaaccaccatcaccttcaaggcctccgctggcggcgacctggaaatcaccacacacagcttcaattgcaggggcgagttcttctactgcgacaccagcgacctgttcaactccagcagattcaacaatagcagcaacgacacgaacgacaccatcacactgccctgcaagatcaagcagatcgtgcggatgtggcagcgcgtgggccagtgtatgtatgcccctcctatcgccggcaacatcacatgcagaagcaatatcaccggcctgctgctcaccagagatggcggcggaaacaacaccaacgagacagaaaccttccggcctgccggcggagacatgagagacaattggagaagcgagctgtacaagtacaaggtggtcaagatcaaccctctgggcgtcgcccctaccagatgtagaagaagagtcgtcggcagacggcggaggcggagagctattggaatgggagctgtgctgctgggctttctgggaacagccggctctacaatgggagccgcctctatcaccctgaccgtgcaagttcgacagctgctgagcggcaaccctgactggctgcctgatatgacagtgtggggaatcaagcagctgcaggccagaatcctggccgtggaaagatacctgaaggaccagcagctcctcggaatctggggctgttctggcaagctgatctgctgcaccaatgtgccctggaacgccagctggtccaacagaagctacaacgacatctgggagaacctgacctgggtcgagtgggagagagagatctccaactacacccagcagatctacagcctgctggaagagagccagaaccagcaagagaagaacgagcaggacctgctggccctggactgataaggatcc

SEQ ID NO: 181 - DNA, 1989 bp, synthetic construct
  source 1..1989: mol_type = other DNA
gtcgacgccaccatgcctatgggatctctgcagcctctggccacactgtacctgctgggaatgctggtggctagcgtgctggcctccaacaatctgtgggtcaccgtgtactacggcgtgcccgtttgggaagatgcctgcacacctctgttctgtgcctccgatgccaaggcctacagcaccaagagcagaaacgtgtgggccacctgttgctgcgtgcccacagatccatctccacaagagatcagcctggacaacgtgaccgagaacttcaacatgtggaagaacaacatggtcgagcagatgcacgaggacatcatcagcctgtgggacgagagcctgaagccttgcgtgaagctgacccctctgtgcgtgaccctgaactgcaccaacgtgaacaacagcagcgccaccaacaactccatggtggacgatagagagggcctgaagaactgcagcttcaacatcaccaccgagctgcgggacaagaagaagcaagagcacgccctgttctaccggctggacatcgtgcccatcaacggcaacagcaactccaacagctccgtgggcgactacagactgatcaactgcaacgtgtccaccatcaagcaggcctgtcctaagatgagcttcgaccccattcctatccactactgtgcccctgccggcttcgccatcctgaagtgcagagacaagaagttcaacggcaccggcagctgcaagaatgtgtccaccgtgcagtgtacccacggcatcaagcctgtgatcagcacccagctgctgctgaatggctctgtggccgaagaggaaatcatgatccgcagcgagaactttaccaacaacgccaagaacatcatcgtccagttcaacaagaccatcgacatcatgtgcacccggcctaacaacaacacccggaagtccatcagcctcggacctggacaagcttggtacgccaccggcgacatcatcggcaatatcagacaggcccactgcaacatctctggcgccgactggggcaacatgatcagaaatgtgtccgagaagctgaaagagatcttcaacaaaaccaccatcaccttcaaggcctccgctggcggcgacctggaaatcaccacacacagcttcaattgcaggggcgagttcttctactgcgacaccagcgacctgttcaactccagcagattcaacaatagcagcaacgacacgaacgacaccatcacactgccctgcaagatcaagcagatcgtgcggatgtggcagcgcgttggccaggctatgtatgcccctcctatcgccggcaacatcacatgcagaagcaatatcaccggcctgctgctcaccagagatggcggcggaaacaacaccaacgagacagagacatttcggcctgccggcggagacatgagagacaattggagaagcgagctgtacaagtacaaggtggtcaagatcaaccctctgggcgtcgcccctaccagatgtagaagaagagtcgtcggcagacggcggaggcggagagctattggaatgggagctgtgctgctgggctttctgggaacagccggctctacaatgggagccgccagcatgacactgaccgtgcaagttcgacagctgctgagcggaatcgtgcagcagcagagcaactgtctgagagcccctgagtgccagcagtgtctgctgcaactgacagtgtggggaatcaagcagctgcaggccagaatcctggccgtggaaagatacctgaaggaccagcagctcctcggaatctggggctgttctggcaagctgatctgctgcaccaatgtgccctggaacgccagctggtccaacagaagctacaacgacatctgggagaacctgacctgggtcgagtgggagagagagatctccaactacacccagcagatctacagcctgctggaagagagccagaaccagcaagagaagaacgagcaggacctgctggccctggactgataaggatcc

SEQ ID NO: 182 - DNA, 1989 bp, synthetic construct
  source 1..1989: mol_type = other DNA
gtcgacgccaccatgcctatgggatctctgcagcctctggccacactgtacctgctgggaatgctggtggctagcgtgctggcctccaacaatctgtgggtcaccgtgtactacggcgtgcccgtttgggaagatgccgagacacctctgttctgtgcctccgatgccaaggcctacagcaccgagaagcacaacgtgtgggccactcatgcctgcgtgccaaccgatccatctcctcaagagatcagcctggacaacgtgaccgagaacttcaacatgtggaagaacaacatggtcgagcagatgcacaccgacatcatcagcctgtgggacgagagcctgaagccttgcgtgaagctgacccctctgtgcgtgaccctgaactgcaccaacgtgaacaacagcagcgccaccaacaactccatggtggacgatagagagggcctgaagaactgcagcttcaacatcaccaccgagctgcgggacaagaaaaaacaggtgcacgccctgttctaccggctggacatcgtgcccatcaacggcaacagcaactccaacagctccgtgggcgactacagactgatcaactgcaacgtgtccaccatcaagcaggcctgtcctaagatgagcttcgaccccattcctatccactactgtgcccctgccggcttcgccatcctgaagtgcagagacaagaagttcaacggcaccggcagctgcaagaatgtgtccaccgtgcagtgtacccacggcatcaagcctgtgatcagcacccagctgctgctgaatggctctgtggccgaagaggaaatcatgatccgcagcgagaactttaccaacaacgccaagaacatcatcgtccagttcaacaagaccatcgacatcatgtgcacccggcctaacaacaacaccagaaagagcatcagactcggcccaggccaggccatctatgccacaggcgatatcatcggcaacatccggcaggcccactgcaatatctctggcgccgattggggcaacatgatccggaacgtgtccgaaaagctgaaagagatcttcaacaaaaccaccatcaccttcaaggcctccgctggcggcgacctggaaatcaccacacacagcttcaattgcaggggcgagttcttctactgcgacaccagcgacctgttcaactccagcagattcaacaatagcagcaacgacacgaacgacaccatcacactgccctgcaagatcaagcagatcgtgcggatgtggcagcgcgttggccaggctatgtatgctcctcctatcgccggcaatatcacctgtcggagcaacattaccggcctgctgctcaccagagatggcggcggaaacaataccaacgagacagaaaccttccggccagccggcggagacatgagagacaattggagaagcgagctgtacaagtacaaggtggtcaagatcaaccctctgggcgtcgcccctaccagatgtagaagaagagtcgtcggcagacggcggaggcggagagctattggaatgggagctgtgctgctgggctttctgggaacagccggctctacaatgggagccgcctctatcaccctgaccgtgcaagtgcgcaatctgctgtctggcatcgtgcagcagcagagcaacctgctgagagcccctgaagctcagcagcatctcctgcagctgacagtgtggggaatcaagcagctccaggccagaatcctggccgtcgagagatacctgagggaccagcagctcctcggaatctggggctgttctggcaagctgatctgctgcaccaatgtgccctggaacgccagctggtccaacagaagctacaacgacatctgggagaacctgacctgggtcgagtgggagagagagatctccaactacacccagcagatctacagcctgctggaagagagccagaaccagcaagagaagaacgagcaggacctgctggccctggactgataaggatcc

SEQ ID NO: 183 - DNA, 2022 bp, synthetic construct
  source 1..2022: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagagccgagaatctgtgggtcacagtgtactatggcgtgcccgtgtgggaagatgccgacacacctctgttttgtgccagcgacgccaaggcctacagcaccgagtctcacaatgtgtgggccactcacgcctgcgtgccaaccgatccatctcctcaagagatcagcctggacaacgtgaccgagaacttcaacatgtggaagaacaacatggtcgagcagatgcacgaggacatcatcagcctgtgggacgagagcctgaagccttgcgtgaagctgacccctctgtgcgtgaccctgaactgcaccaacgtgaacaacagcagcgccaccaacaactccatggtggacgatagagagggcctgaagaactgcagcttcaacatcaccaccgagctgcgggacaagaagaagcaagagcacgccctgttctaccggctggacatcgtgcccatcaacggcaacagcaactccaacagctccgtgggcgactacagactgatcaactgcaacgtgtccacatgcaagcaggcctgtcctaagatgagcttcgaccccattcctatccactactgtgcccctgccggcttcgccatcctgaagtgcagagacaagaagttcaacggcaccggcagctgcaagaatgtgtccaccgtgcagtgtacccacggcatcaagcctgtgatcagcacccagctgctgctgaatggctctgtggccgaagaggaaatcatgatccgcagcgagaactttaccaacaacgccaagaacatcatcgtccagttcaacaagaccatcgacatcatgtgcacccggcctaacaacaacacccggaagtccatcagcctcggacctggacaggccatctatgccaccggcgacatcatcggcaatatcagacaggcccactgcaacatctctggcgccgactggggcaacatgatcagaaatgtgtccgagaagctgaaagagatcttcaacaaaaccaccatcaccttcaaggcctccgctggcggcgacctggaaatcaccacacacagcttcaattgcaggggcgagttcttctactgcgacaccagcgacctgttcaactccagcagattcaacaatagcagcaacgacacgaacgacaccatcacactgccctgcaagatcaagcagatcgtgcggatgtggcagcgcgtgggccagtgtatgtatgcccctcctatcgccggcaacatcacatgcagaagcaatatcaccggcctgctgctcaccagagatggcggcggaaacaacaccaacgagacagaaaccttccggcctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggctgtggaaagatacctgagggaccagcagctcctcggaatctggggctgttctggcaagctgatctgctgcaccaatgtgccctggaacagctcctggtccaaccggaatctgagcgagatctgggacaatatgacctggctgcagtgggacaaagagattagcaactacacccagatcatctacggactgctggaagagagccagaaccagcaagagaaaaacgagcaggacctgctggccctggactgataaggatcc

SEQ ID NO: 184 - DNA, 2022 bp, synthetic construct
  source 1..2022: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagatccaacaatctgtgggtcaccgtgtactacggcgtgcccgtttgggaagatgccgacacacctctgttctgtgcctccgatgccaaggcctacagcaccgagtctcacaacgtgtgggccactcatgcctgcgtgccaaccgatccatctcctcaagagatcagcctggacaacgtgaccgagaacttcaacatgtggaagaacaacatggtcgagcagatgcacgaggacatcatcagcctgtgggacgagagcctgaagccttgcgtgaagctgacccctctgtgcgtgaccctgaactgcaccaacgtgaacaacagcagcgccaccaacaactccatggtggacgatagagagggcctgaagaactgcagcttcaacatcaccaccgagctgcgggacaagaagaagcaagagcacgccctgttctaccggctggacatcgtgcccatcaacggcaacagcaactccaacagctccgtgggcgactacagactgatcaactgcaacgtgtccaccatcaagcaggcctgtcctaagatgagcttcgaccccattcctatccactactgtgcccctgccggcttcgccatcctgaagtgcagagacaagaagttcaacggcaccggcagctgcaagaatgtgtccaccgtgcagtgtacccacggcatcaagcctgtgatcagcacccagctgctgctgaatggctctgtggccgaagaggaaatcatgatccgcagcgagaactttaccaacaacgccaagaacatcatcgtccagttcaacaagaccatcgacatcatgtgcacccggcctaacaacaacacccggaagtccatcagcctcggacctggacaggccatctatgccaccggcgacatcatcggcaatatcagacaggcccactgcaacatctctggcgccgactggggcaacatgatcagaaatgtgtccgagaagctgaaagagatcttcaacaaaaccaccatcaccttcaaggcctccgctggcggcgacctggaaatcaccacacacagcttcaattgcaggggcgagttcttctactgcgacaccagcgacctgttcaactccagcagattcaacaatagcagcaacgacacgaacgacaccatcacactgccctgcaagatcaagcagatcgtgcggatgtggcagcgcgttggccaggctatgtatgcccctcctatcgccggcaacatcacatgcagaagcaatatcaccggcctgctgctcaccagagatggcggcggaaacaacaccaacgagacagaaaccttccggcctgccggcggagacatgagagacaattggagaagcgagctgtacaagtacaaggtggtcaagatcaaccctctgggcgtcgcccctaccagatgtagaagaagagtcgtcggcagacggcggaggcggagagctattggaatgggagctgtgctgctgggctttctgggaacagccggctctacaatgggagccgcctctatcaccctgaccgtgcaagttcgacagctgctgtctggcatcgtgcagcagcagagcaacctgctgagagcccctgaagctcagcagcatctcctgcagctgacagtgtggggaatcaagcagctccaggccagaatcctggccgtcgagagatacctgaaggaccagcagctcctcggaatctggggctgttctggcaagctgatctgctgcaccaatgtgccctggaacgccagctggtccaacagaagctacaacgacatctgggagaacctgacctgggtcgagtgggagagagagatctccaactacacccagcagatctacagcctgctggaagagagccagaaccagcaagagaagaacgagcaggacctgctggccctggactgataaggatcc

SEQ ID NO: 185 - DNA, 2022 bp, synthetic construct
  source 1..2022: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagatccaacaatctgtgggtcaccgtgtactacggcgtgcccgtttgggaagatgccgacacacctctgttctgtgcctccgatgccaaggcctacagcaccgagtctcacaacgtgtgggccactcatgcctgcgtgccaaccgatccatctcctcaagagatcagcctggacaacgtgaccgagaacttcaacatgtggaagaacaacatggtcgagcagatgcacgaggacatcatcagcctgtgggacgagagcctgaagccttgcgtgaagctgacccctctgtgcgtgaccctgaactgcaccaacgtgaacaacagcagcgccaccaacaactccatggtggacgatagagagggcctgaagaactgcagcttcaacatcaccaccgagctgcgggacaagaagaagcaagagcacgccctgttctaccggctggacatcgtgcccatcaacggcaacagcaactccaacagctccgtgggcgactacagactgatcaactgcaacgtgtccacctgcaagcaggcctgtcctaagatgagcttcgaccccattcctatccactactgtgcccctgccggcttcgccatcctgaagtgcagagacaagaagttcaacggcaccggcagctgcaagaatgtgtccaccgtgcagtgtacccacggcatcaagcctgtgatcagcacccagctgctgctgaatggctctgtggccgaagaggaaatcatgatccgcagcgagaactttaccaacaacgccaagaacatcatcgtccagttcaacaagaccatcgacatcatgtgcacccggcctaacaacaacacccggaagtccatcagcctcggacctggacaggccatctatgccaccggcgacatcatcggcaatatcagacaggcccactgcaacatctctggcgccgactggggcaacatgatcagaaatgtgtccgagaagctgaaagagatcttcaacaaaaccaccatcaccttcaaggcctccgctggcggcgacctggaaatcaccacacacagcttcaattgcaggggcgagttcttctactgcgacaccagcgacctgttcaactccagcagattcaacaatagcagcaacgacacgaacgacaccatcacactgccctgcaagatcaagcagatcgtgcggatgtggcagcgcgttggccagtgcatgtatgcccctcctatcgccggcaacatcacatgcagaagcaatatcaccggcctgctgctcaccagagatggcggcggaaacaacaccaacgagacagaaaccttccggcctgccggcggagacatgagagacaattggagaagcgagctgtacaagtacaaggtggtcaagatcaaccctctgggcgtcgcccctaccagatgtagaagaagagtcgtcggcagacggcggaggcggagagctattggaatgggagctgtgctgctgggctttctgggaacagccggctctacaatgggagccgcctctatcaccctgaccgtgcaagttcgacagctgctgtctggcatcgtgcagcagcagagcaacctgctgagagcccctgaagctcagcagcatctcctgcagctgacagtgtggggaatcaagcagctccaggccagaatcctggccgtcgagagatacctgaaggaccagcagctcctcggaatctggggctgttctggcaagctgatctgctgcaccaatgtgccctggaacgccagctggtccaacagaagctacaacgacatctgggagaacctgacctgggtcgagtgggagagagagatctccaactacacccagcagatctacagcctgctggaagagagccagaaccagcaagagaagaacgagcaggacctgctggccctggactgataaggatcc

SEQ ID NO: 186 - DNA, 1983 bp, synthetic construct
  source 1..1983: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagatccaacaatctgtgggtcaccgtgtactacggcgtgcccgtttgggaagatgccgacacacctctgttctgtgcctccgatgccaaggcctacagcaccgagtctcacaacgtgtgggccactcatgcctgcgtgccaaccgatccatctcctcaagagatcagcctggacaacgtgaccgagaacttcaacatgtggaagaacaacatggtcgagcagatgcacgaggacatcatcagcctgtgggacgagagcctgaagccttgcgtgaagctgacccctctgtgcgtgaccctgaactgcaccaacgtgaacaacagcagcgccaccaacaactccatggtggacgatagagagggcctgaagaactgcagcttcaacatcaccaccgagctgcgggacaagaagaagcaagagcacgccctgttctaccggctggacatcgtgcccatcaacggcaacagcaactccaacagctccgtgggcgactacagactgatcaactgcaacgtgtccacatgcaagcaggcctgtcctaagatgagcttcgaccccattcctatccactactgtgcccctgccggcttcgccatcctgaagtgcagagacaagaagttcaacggcaccggcagctgcaagaatgtgtccaccgtgcagtgtacccacggcatcaagcctgtgatcagcacccagctgctgctgaatggctctgtggccgaagaggaaatcatgatccgcagcgagaactttaccaacaacgccaagaacatcatcgtccagttcaacaagaccatcgacatcatgtgcacccggcctaacaacaacacccggaagtccatcagcctcggacctggacaggccatctatgccaccggcgacatcatcggcaatatcagacaggcccactgcaacatctctggcgccgactggggcaacatgatcagaaatgtgtccgagaagctgaaagagatcttcaacaaaaccaccatcaccttcaaggcctccgctggcggcgacctggaaatcaccacacacagcttcaattgcaggggcgagttcttctactgcgacaccagcgacctgttcaactccagcagattcaacaatagcagcaacgacacgaacgacaccatcacactgccctgcaagatcaagcagatcgtgcggatgtggcagcgcgtgggccagtgtatgtatgcccctcctatcgccggcaacatcacatgcagaagcaatatcaccggcctgctgctcaccagagatggcggcggaaacaacaccaacgagacagaaaccttccggcctgccggcggagacatgagagacaattggagaagcgagctgtacaagtacaaggtggtcaagatcaaccctctgggcgtcgcccctaccagatgtagaagaagagtcgtcggcagacggcggaggcggagagctattggaatgggagctgtgctgctgggctttctgggaacagccggctctacaatgggagccgcctctatcaccctgaccgtgcaagttcgacagctgctgagcggcaaccctgactggctgcctgatatgacagtgtggggaatcaagcagctgcaggccagaatcctggccgtggaaagatacctgaaggaccagcagctcctcggaatctggggctgttctggcaagctgatctgctgcaccaatgtgccctggaacgccagctggtccaacagaagctacaacgacatctgggagaacctgacctgggtcgagtgggagagagagatctccaactacacccagcagatctacagcctgctggaagagagccagaaccagcaagagaagaacgagcaggacctgctggccctggactgataaggatcc

SEQ ID NO: 187 - DNA, 2022 bp, synthetic construct
  source 1..2022: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagatccaacaatctgtgggtcaccgtgtactacggcgtgcccgtttgggaagatgcctgcacacctctgttctgtgcctccgatgccaaggcctacagcaccaagagcagaaacgtgtgggccacctgttgctgcgtgcccacagatccatctccacaagagatcagcctggacaacgtgaccgagaacttcaacatgtggaagaacaacatggtcgagcagatgcacgaggacatcatcagcctgtgggacgagagcctgaagccttgcgtgaagctgacccctctgtgcgtgaccctgaactgcaccaacgtgaacaacagcagcgccaccaacaactccatggtggacgatagagagggcctgaagaactgcagcttcaacatcaccaccgagctgcgggacaagaagaagcaagagcacgccctgttctaccggctggacatcgtgcccatcaacggcaacagcaactccaacagctccgtgggcgactacagactgatcaactgcaacgtgtccaccatcaagcaggcctgtcctaagatgagcttcgaccccattcctatccactactgtgcccctgccggcttcgccatcctgaagtgcagagacaagaagttcaacggcaccggcagctgcaagaatgtgtccaccgtgcagtgtacccacggcatcaagcctgtgatcagcacccagctgctgctgaatggctctgtggccgaagaggaaatcatgatccgcagcgagaactttaccaacaacgccaagaacatcatcgtccagttcaacaagaccatcgacatcatgtgcacccggcctaacaacaacacccggaagtccatcagcctcggacctggacaagcttggtacgccaccggcgacatcatcggcaatatcagacaggcccactgcaacatctctggcgccgactggggcaacatgatcagaaatgtgtccgagaagctgaaagagatcttcaacaaaaccaccatcaccttcaaggcctccgctggcggcgacctggaaatcaccacacacagcttcaattgcaggggcgagttcttctactgcgacaccagcgacctgttcaactccagcagattcaacaatagcagcaacgacacgaacgacaccatcacactgccctgcaagatcaagcagatcgtgcggatgtggcagcgcgttggccaggctatgtatgcccctcctatcgccggcaacatcacatgcagaagcaatatcaccggcctgctgctcaccagagatggcggcggaaacaacaccaacgagacagagacatttcggcctgccggcggagacatgagagacaattggagaagcgagctgtacaagtacaaggtggtcaagatcaaccctctgggcgtcgcccctaccagatgtagaagaagagtcgtcggcagacggcggaggcggagagctattggaatgggagctgtgctgctgggctttctgggaacagccggctctacaatgggagccgccagcatgacactgaccgtgcaagttcgacagctgctgagcggaatcgtgcagcagcagagcaactgtctgagagcccctgagtgccagcagtgtctgctgcaactgacagtgtggggaatcaagcagctgcaggccagaatcctggccgtggaaagatacctgaaggaccagcagctcctcggaatctggggctgttctggcaagctgatctgctgcaccaatgtgccctggaacgccagctggtccaacagaagctacaacgacatctgggagaacctgacctgggtcgagtgggagagagagatctccaactacacccagcagatctacagcctgctggaagagagccagaaccagcaagagaagaacgagcaggacctgctggccctggactgataaggatcc

SEQ ID NO: 188 - DNA, 2022 bp, synthetic construct
  source 1..2022: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagatccaacaatctgtgggtcaccgtgtactacggcgtgcccgtttgggaagatgccgagacacctctgttctgtgcctccgatgccaaggcctacagcaccgagaagcacaacgtgtgggccactcatgcctgcgtgccaaccgatccatctcctcaagagatcagcctggacaacgtgaccgagaacttcaacatgtggaagaacaacatggtcgagcagatgcacaccgacatcatcagcctgtgggacgagagcctgaagccttgcgtgaagctgacccctctgtgcgtgaccctgaactgcaccaacgtgaacaacagcagcgccaccaacaactccatggtggacgatagagagggcctgaagaactgcagcttcaacatcaccaccgagctgcgggacaagaaaaaacaggtgcacgccctgttctaccggctggacatcgtgcccatcaacggcaacagcaactccaacagctccgtgggcgactacagactgatcaactgcaacgtgtccaccatcaagcaggcctgtcctaagatgagcttcgaccccattcctatccactactgtgcccctgccggcttcgccatcctgaagtgcagagacaagaagttcaacggcaccggcagctgcaagaatgtgtccaccgtgcagtgtacccacggcatcaagcctgtgatcagcacccagctgctgctgaatggctctgtggccgaagaggaaatcatgatccgcagcgagaactttaccaacaacgccaagaacatcatcgtccagttcaacaagaccatcgacatcatgtgcacccggcctaacaacaacaccagaaagagcatcagactcggcccaggccaggccatctatgccacaggcgatatcatcggcaacatccggcaggcccactgcaatatctctggcgccgattggggcaacatgatccggaacgtgtccgaaaagctgaaagagatcttcaacaaaaccaccatcaccttcaaggcctccgctggcggcgacctggaaatcaccacacacagcttcaattgcaggggcgagttcttctactgcgacaccagcgacctgttcaactccagcagattcaacaatagcagcaacgacacgaacgacaccatcacactgccctgcaagatcaagcagatcgtgcggatgtggcagcgcgttggccaggctatgtatgctcctcctatcgccggcaatatcacctgtcggagcaacattaccggcctgctgctcaccagagatggcggcggaaacaataccaacgagacagaaaccttccggccagccggcggagacatgagagacaattggagaagcgagctgtacaagtacaaggtggtcaagatcaaccctctgggcgtcgcccctaccagatgtagaagaagagtcgtcggcagacggcggaggcggagagctattggaatgggagctgtgctgctgggctttctgggaacagccggctctacaatgggagccgcctctatcaccctgaccgtgcaagtgcgcaatctgctgtctggcatcgtgcagcagcagagcaacctgctgagagcccctgaagctcagcagcatctcctgcagctgacagtgtggggaatcaagcagctccaggccagaatcctggccgtcgagagatacctgagggaccagcagctcctcggaatctggggctgttctggcaagctgatctgctgcaccaatgtgccctggaacgccagctggtccaacagaagctacaacgacatctgggagaacctgacctgggtcgagtgggagagagagatctccaactacacccagcagatctacagcctgctggaagagagccagaaccagcaagagaagaacgagcaggacctgctggccctggactgataaggatcc

SEQ ID NO: 189 - DNA, 1956 bp, synthetic construct
  source 1..1956: mol_type = other DNA
gtcgacgccaccatgggatctctgcaacctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacgatcccgaagtgcacaatgtgtgggccactcacgcctgcgtgcccaccgatcctaatcctcaagagatgttcctggaaaacgtgaccgagaacttcaacatgtggaagaacgacatggtcgagcagatgcaccaggacatcatcagcctgtggtcccagagcctgaagccttgcgtgaagctgacccctctgtgcgtgaccctgaattgcagcgacgtgatcaacagcaccggcaccatgaagaactgcagcttcagcgtgaccaccgagctgcgggacagaaagcagaagaagcacgccctgttctaccggctggacatcgtgcccctggacgagaacgacaacagcagcaaggactacagactgatcaactgcaacaccagcacatgcacccaggcctgtcctaaagtgaccttcgatcccattcctatccactactgtgcccctgccggctacgccatcctgaagtgcaacaacaagaccttcaacggcacaggcccctgcaacaacgtgtccaccgtgcagtgtacccacggcatcaagccagtggtgtctacccagctgctgctgaatggctctctggccgaggaagaagtgatcatcagaagcgagaacctgaccgacaacgtcaagaccatcatcgtgcagctgaaagaacccgtgggcatcaattgcacccggcctaacaacaacatccggcagagcatcagaatcggccctggccagacctttttcgccaccggcgatatcatcggcgacatcagagaggcccactgcaatatcagcagcaccaagtggcgggaaaccctgcagcaagttggaggcaagctggccaagtacttcaacaacaaaacgatcaagttcacccagcactctggcggcgacctggaaatcaccacacacagcttcaactgcagaggcgagttcttctactgtaataccagccagctgttcaactccacctacaacgagacaggctccatcaatggcaccggcaatagcaccatcaagctgccctgccggatcaagcagatcatcaatatgtggcaaggcgtgggccagtgtatgtacgcccctcctatcgccggcaacatcatctgcagaagcaagatcaccggcctgctgctcacaagagatggcggcatcaacaagagcgaggaaatcttcagacccggcggaggcaacatgaaggacaactggcgggacaattggcggagcgagctgtacaagtacaaggtggtcaagatcgagcccctgggcgtcgcacctacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtccagcagcagagcaatctgctcagagcccctgaggctcagcagcatctcctgaaactgaccgtgtggggaatcaagcagctgcaggccagagttctggccgtggaaagatacctgagggaccagcagctcctcggcatctggggctgttctggcaagctgatctgctgcaccaacgtgccctggaacagctcctggtccaacagaaacctgagcgagatctgggacaacatgacctggctgcagtgggacaaagagatcagcaactacacccagatcatctacggactgctggaagagagccagaaccagcaagagaaaaacgagcaggacctgctggccctggactgataaggatcc

SEQ ID NO: 190 - DNA, 1941 bp, synthetic construct
  source 1..1941: mol_type = other DNA
gtcgacgccaccatgggatctctgcaacctctggccacactgtacctgctgggaatgctggtggctagcgtgctggccaatctgtgggtcacagtgtactacggcgtgcccgtgtggaaagaggccaagaccacactgttctgtgccagcgacgccaaggcctacgatcccgaagtgcacaatgtgtgggccactcacgcctgcgtgcccaccgatcctaatcctcaagagatgttcctggaaaacgtgaccgagaacttcaacatgtggaagaacgacatggtcgagcagatgcaccaggacatcatcagcctgtggtcccagagcctgaagccttgcgtgaagctgacccctctgtgcgtgaccctgaattgcagcgacgtgatcaacagcaccggcaccatgaagaactgcagcttcagcgtgaccaccgagctgcgggacagaaagcagaagaagcacgccctgttctaccggctggacatcgtgcccctggacgagaacgacaacagcagcaaggactacagactgatcaactgcaacaccagcacatgcacccaggcctgtcctaaagtgaccttcgatcccattcctatccactactgtgcccctgccggctacgccatcctgaagtgcaacaacaagaccttcaacggcacaggcccctgcaacaacgtgtccaccgtgcagtgtacccacggcatcaagccagtggtgtctacccagctgctgctgaatggctctctggccgaggaagaagtgatcatcagaagcgagaacctgaccgacaacgtcaagaccatcatcgtgcagctgaaagaacccgtgggcatcaattgcacccggcctaacaacaacatccggcagagcatcagaatcggccctggccagacctttttcgccaccggcgatatcatcggcgacatcagagaggcccactgcaatatcagcagcaccaagtggcgggaaaccctgcagcaagttggaggcaagctggccaagtacttcaacaacaaaacgatcaagttcacccagcactctggcggcgacctggaaatcaccacacacagcttcaactgcagaggcgagttcttctactgtaataccagccagctgttcaactccacctacaacgagacaggctccatcaatggcaccggcaatagcaccatcaagctgccctgccggatcaagcagatcatcaatatgtggcaaggcgtgggccagtgtatgtacgcccctcctatcgccggcaacatcatctgcagaagcaagatcaccggcctgctgctcacaagagatggcggcatcaacaagagcgaggaaatcttcagacccggcggaggcaacatgaaggacaattggagaagcgagctgtacaagtacaaggtggtggaaatcaagcccctgggcgtcgcccctacaaagtgcaagagaagagtggtggaacggcggagaaggcggagagctgttgttggactgggagccgtgttcctgggctttcttggagccgccggatctacaatgggagccgcctctatcaccctgaccgtgcaggctagacagctgctgagcggaattgtccagcagcagaacaacctgctgagagcccctgaagcacagcaacacatgctgcagctgaccgtgtggggaatcaaacagctgcaggccagagtcctggccatcgagagatacctgaaggaccagcagctcctcggcatctggggctgttctggcaagctgatctgctgtactgccgtgccttggaacagctcctggtccaacaagtcccaagaggacatctgggacaacatgacctggatgcagtgggacagagagatcagcaactacaccaacaccatctaccgcctgctggaagatagccagacacagcaagagaaaaacgagaaggacctgctggccctggactgatgaggatcc

SEQ ID NO: 191 - DNA, 1941 bp, synthetic construct
  source 1..1941: mol_type = other DNA
gtcgacgccaccatgggatctctgcaacctctggccacactgtacctgctgggaatgctggtggctagcgtgctggccaatctgtgggtcacagtgtactacggcgtgcccgtgtggaaagaggcctgcaccacactgttttgtgccagcgacgccaaggcctacgatcccaaagtgcggaatgtgtgggccactcactgctgcgtgcccaccgatcctaatcctcaagagatgttcctggaaaacgtgaccgagaacttcaacatgtggaagaacgacatggtcgagcagatgcaccaggacatcatcagcctgtggtcccagagcctgaagccttgcgtgaagctgacccctctgtgcgtgaccctgaattgcagcgacgtgatcaacagcaccggcaccatgaagaactgcagcttcagcgtgaccaccgagctgcgggacagaaagcagaagaagcacgccctgttctaccggctggacatcgtgcccctggacgagaacgacaacagcagcaaggactacagactgatcaactgcaacaccagcacatgcacccaggcctgtcctaaagtgaccttcgatcccattcctatccactactgtgcccctgccggctacgccatcctgaagtgcaacaacaagaccttcaacggcacaggcccctgcaacaacgtgtccaccgtgcagtgtacccacggcatcaagccagtggtgtctacccagctgctgctgaatggctctctggccgaggaagaagtgatcatcagaagcgagaacctgaccgacaacgtcaagaccatcatcgtgcagctgaaagaacccgtgggcatcaattgcacccggcctaacaacaacatccggcagagcatcagaatcggccctggccagacttggtttgccaccggcgatatcatcggcgacatcagagaggcccactgcaatatcagcagcaccaagtggcgggaaaccctgcagcaagttggaggcaagctggccaagtacttcaacaacaaaacgatcaagttcacccagcactctggcggcgacctggaaatcaccacacacagcttcaactgcagaggcgagttcttctactgtaataccagccagctgttcaactccacctacaacgagacaggctccatcaatggcaccggcaatagcaccatcaagctgccctgccggatcaagcagatcatcaatatgtggcaaggcgtgggccagtgtatgtacgcccctcctatcgccggcaacatcatctgcagaagcaagatcaccggcctgctgctcacaagagatggcggcatcaacaagagcgaggaaatcttcagacccggcggaggcaacatgaaggacaattggagaagcgagctgtacaagtacaaggtggtggaaatcaagcccctgggcgtcgcccctacaaagtgcaagagaagagtggtggaacggcggagaaggcggagagctgttgttggactgggagccgtgttcctgggctttcttggagccgccggatctacaatgggagccgcctctatgaccctgaccgtgcaggctagacagctgctgagcggaattgtccagcagcagaacaactgcctgagagcccctgagtgccagcagtgtatgctgcagctgacagtgtggggaatcaagcagctgcaggccagagttctggccatcgagagatacctgaaggaccagcagctcctcggcatctggggctgttctggcaagctgatctgctgtactgccgtgccttggaacagctcctggtccaacaagtcccaagaggacatctgggacaacatgacctggatgcagtgggacagagagatcagcaactacaccaacaccatctaccgcctgctggaagatagccagacacagcaagagaaaaacgagaaggacctgctggccctggactgatgaggatcc

SEQ ID NO: 192 - DNA, 1902 bp, synthetic construct
  source 1..1902: mol_type = other DNA
gtcgacgccaccatgggatctctgcaacctctggccacactgtacctgctgggaatgctggtggctagcgtgctggccaatctgtgggtcacagtgtactacggcgtgcccgtgtggaaagaggccaagaccacactgttctgtgccagcgacgccaaggcctacgatcccgaagtgcacaatgtgtgggccactcacgcctgcgtgcccaccgatcctaatcctcaagagatgttcctggaaaacgtgaccgagaacttcaacatgtggaagaacgacatggtcgagcagatgcaccaggacatcatcagcctgtggtcccagagcctgaagccttgcgtgaagctgacccctctgtgcgtgaccctgaattgcagcgacgtgatcaacagcaccggcaccatgaagaactgcagcttcagcgtgaccaccgagctgcgggacagaaagcagaagaagcacgccctgttctaccggctggacatcgtgcccctggacgagaacgacaacagcagcaaggactacagactgatcaactgcaacaccagcacatgcacccaggcctgtcctaaagtgaccttcgatcccattcctatccactactgtgcccctgccggctacgccatcctgaagtgcaacaacaagaccttcaacggcacaggcccctgcaacaacgtgtccaccgtgcagtgtacccacggcatcaagccagtggtgtctacccagctgctgctgaatggctctctggccgaggaagaagtgatcatcagaagcgagaacctgaccgacaacgtcaagaccatcatcgtgcagctgaaagaacccgtgggcatcaattgcacccggcctaacaacaacatccggcagagcatcagaatcggccctggccagacctttttcgccaccggcgatatcatcggcgacatcagagaggcccactgcaatatcagcagcaccaagtggcgggaaaccctgcagcaagttggaggcaagctggccaagtacttcaacaacaaaacgatcaagttcacccagcactctggcggcgacctggaaatcaccacacacagcttcaactgcagaggcgagttcttctactgtaataccagccagctgttcaactccacctacaacgagacaggctccatcaatggcaccggcaatagcaccatcaagctgccctgccggatcaagcagatcatcaatatgtggcaaggcgtgggccagtgtatgtacgcccctcctatcgccggcaacatcatctgcagaagcaagatcaccggcctgctgctcacaagagatggcggcatcaacaagagcgaggaaatcttcagacccggcggaggcaacatgaaggacaattggagaagcgagctgtacaagtacaaggtggtggaaatcaagcccctgggcgtcgcccctacaaagtgcaagagaagagtggtggaacggcggagaaggcggagagctgttgttggactgggagccgtgttcctgggctttcttggagccgccggatctacaatgggagccgcctctatcaccctgaccgtgcaggctagacagctgctgagcggcaatcctgactggctgcctgatatgaccgtgtggggaatcaagcagctgcaggccagagttctggccatcgagagatacctgaaggaccagcagctcctcggcatctggggctgttctggcaagctgatctgctgtactgccgtgccttggaacagctcctggtccaacaagtcccaagaggacatctgggacaacatgacctggatgcagtgggacagagagatcagcaactacaccaacaccatctaccgcctgctggaagatagccagacacagcaagagaaaaacgagaaggacctgctggccctggactgatgaggatcc

SEQ ID NO: 193 - DNA, 1941 bp, synthetic construct
  source 1..1941: mol_type = other DNA
gtcgacgccaccatgggatctctgcaacctctggccacactgtacctgctgggaatgctggtggctagcgtgctggccaatctgtgggtcacagtgtactacggcgtgcccgtgtggaaggatgccgagacaacactgttctgtgccagcgacgccaaggcctacgatcccgagaaacacaacgtgtgggccactcacgcctgcgtgccaaccgatcctaatcctcaagagatgttcctggaaaacgtgaccgagaacttcaacatgtggaagaacgacatggtcgagcagatgcacaccgacatcatcagcctgtggtcccagagcctgaagccttgcgtgaagctgacccctctgtgcgtgaccctgaattgcagcgacgtgatcaacagcaccggcaccatgaagaactgcagcttcagcgtgaccaccgagctgcgggacagaaagcagaaagtgcacgccctgttctaccggctggacatcgttcctctggacgagaacgacaacagcagcaaggactacagactgatcaactgcaacaccagcacatgcacccaggcctgtcctaaagtgaccttcgatcccattcctatccactactgtgcccctgccggctacgccatcctgaagtgcaacaacaagaccttcaacggcacaggcccctgcaacaacgtgtccaccgtgcagtgtacccacggcatcaagccagtggtgtctacccagctgctgctgaatggctctctggccgaggaagaagtgatcatcagaagcgagaacctgaccgacaacgtcaagaccatcatcgtgcagctgaaagaacccgtgggcatcaattgcacccggcctaacaacaacatccggcagagcatcagaatcggccctggccagacctttttcgccaccggcgatatcatcggcgacatcagagaggcccactgcaatatcagcagcaccaagtggcgggaaaccctgcagcaagttggaggcaagctggccaagtacttcaacaacaaaacgatcaagttcacccagcactctggcggcgacctggaaatcaccacacacagcttcaactgcagaggcgagttcttctactgtaataccagccagctgttcaactccacctacaacgagacaggctccatcaatggcaccggcaatagcaccatcaagctgccctgccggatcaagcagatcatcaatatgtggcagcgcgtgggccagtgtatgtacgcccctccaatcgccggcaacatcatctgcagaagcaagatcaccggcctgctgctcacaagagatggcggcatcaacaagagcgaggaaatcttcagacccggcggaggcaacatgaaggacaattggagaagcgagctgtacaagtacaaggtggtggaaatcaagcccctgggcgtcgcccctacacggtgcaaaagaagagtggtcgagcggcggagaaggcggagagctgttgttggactgggagccgtgttcctgggctttcttggagccgccggatctacaatgggagccgcctctatcaccctgaccgtgcaggctagaaatctgctgagcggcatcgtccagcagcagagcaatctgctcagagcccctgaggcacagcaacacatgctgcagctgacagtgtggggaatcaagcagctgcaggccagagttctggccatcgagagatacctgagggaccagcagctcctcggcatctggggctgttctggcaagctgatctgctgtactgccgtgccttggaacagctcctggtccaacaagtcccaagaggacatctgggacaacatgacctggatgcagtgggacagagagatcagcaactacaccaacaccatctaccgcctgctggaagatagccagacacagcaagagaagaacgagaaggacctgctggccctggactgatgaggatcc

SEQ ID NO: 194 - DNA, 1995 bp, synthetic construct
  source 1..1995: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagagccgagaatctgtgggtcacagtgtactatggcgtgcccgtgtggaaagaggccaagaccacactgttctgtgccagcgacgccaaggcctacgatcccgaagtgcacaatgtgtgggccactcacgcctgcgtgcccaccgatcctaatcctcaagagatgttcctggaaaacgtgaccgagaacttcaacatgtggaagaacgacatggtcgagcagatgcaccaggacatcatcagcctgtggtcccagagcctgaagccttgcgtgaagctgacccctctgtgcgtgaccctgaattgcagcgacgtgatcaacagcaccggcaccatgaagaactgcagcttcagcgtgaccaccgagctgcgggacagaaagcagaagaagcacgccctgttctaccggctggacatcgtgcccctggacgagaacgacaacagcagcaaggactacagactgatcaactgcaacaccagcacatgcacccaggcctgtcctaaagtgaccttcgatcccattcctatccactactgtgcccctgccggctacgccatcctgaagtgcaacaacaagaccttcaacggcacaggcccctgcaacaacgtgtccaccgtgcagtgtacccacggcatcaagccagtggtgtctacccagctgctgctgaatggctctctggccgaggaagaagtgatcatcagaagcgagaacctgaccgacaacgtcaagaccatcatcgtgcagctgaaagaacccgtgggcatcaattgcacccggcctaacaacaacatccggcagagcatcagaatcggccctggccagacctttttcgccaccggcgatatcatcggcgacatcagagaggcccactgcaatatcagcagcaccaagtggcgggaaaccctgcagcaagttggaggcaagctggccaagtacttcaacaacaaaacgatcaagttcacccagcactctggcggcgacctggaaatcaccacacacagcttcaactgcagaggcgagttcttctactgtaataccagccagctgttcaactccacctacaacgagacaggctccatcaatggcaccggcaatagcaccatcaagctgccctgccggatcaagcagatcatcaatatgtggcaaggcgtgggccagtgtatgtacgcccctcctatcgccggcaacatcatctgcagaagcaagatcaccggcctgctgctcacaagagatggcggcatcaacaagagcgaggaaatcttcagacccggcggaggcaacatgaaggacaactggcgggacaattggcggagcgagctgtacaagtacaaggtggtcaagatcgagcccctgggcgtcgcacctacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtccagcagcagagcaatctgctcagagcccctgaggctcagcagcatctcctgaaactgaccgtgtggggaatcaagcagctgcaggccagagttctggccgtggaaagatacctgagggaccagcagctcctcggcatctggggctgttctggcaagctgatctgctgcaccaacgtgccctggaacagctcctggtccaacagaaacctgagcgagatctgggacaacatgacctggctgcagtgggacaaagagatcagcaactacacccagatcatctacggactgctggaagagagccagaaccagcaagagaaaaacgagcaggacctgctggccctggactgataaggatcc

SEQ ID NO: 195 - DNA, 1980 bp, synthetic construct
  source 1..1980: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagaaatctgtgggtcacagtgtactacggcgtgcccgtgtggaaagaggccaagaccacactgttctgtgccagcgacgccaaggcctacgatcccgaagtgcacaatgtgtgggccactcacgcctgcgtgcccaccgatcctaatcctcaagagatgttcctggaaaacgtgaccgagaacttcaacatgtggaagaacgacatggtcgagcagatgcaccaggacatcatcagcctgtggtcccagagcctgaagccttgcgtgaagctgacccctctgtgcgtgaccctgaattgcagcgacgtgatcaacagcaccggcaccatgaagaactgcagcttcagcgtgaccaccgagctgcgggacagaaagcagaagaagcacgccctgttctaccggctggacatcgtgcccctggacgagaacgacaacagcagcaaggactacagactgatcaactgcaacaccagcacatgcacccaggcctgtcctaaagtgaccttcgatcccattcctatccactactgtgcccctgccggctacgccatcctgaagtgcaacaacaagaccttcaacggcacaggcccctgcaacaacgtgtccaccgtgcagtgtacccacggcatcaagccagtggtgtctacccagctgctgctgaatggctctctggccgaggaagaagtgatcatcagaagcgagaacctgaccgacaacgtcaagaccatcatcgtgcagctgaaagaacccgtgggcatcaattgcacccggcctaacaacaacatccggcagagcatcagaatcggccctggccagacctttttcgccaccggcgatatcatcggcgacatcagagaggcccactgcaatatcagcagcaccaagtggcgggaaaccctgcagcaagttggaggcaagctggccaagtacttcaacaacaaaacgatcaagttcacccagcactctggcggcgacctggaaatcaccacacacagcttcaactgcagaggcgagttcttctactgtaataccagccagctgttcaactccacctacaacgagacaggctccatcaatggcaccggcaatagcaccatcaagctgccctgccggatcaagcagatcatcaatatgtggcaaggcgtgggccagtgtatgtacgcccctcctatcgccggcaacatcatctgcagaagcaagatcaccggcctgctgctcacaagagatggcggcatcaacaagagcgaggaaatcttcagacccggcggaggcaacatgaaggacaattggagaagcgagctgtacaagtacaaggtggtggaaatcaagcccctgggcgtcgcccctacaaagtgcaagagaagagtggtggaacggcggagaaggcggagagctgttgttggactgggagccgtgttcctgggctttcttggagccgccggatctacaatgggagccgcctctatcaccctgaccgtgcaggctagacagctgctgagcggaattgtccagcagcagaacaacctgctgagagcccctgaagcacagcaacacatgctgcagctgaccgtgtggggaatcaaacagctgcaggccagagtcctggccatcgagagatacctgaaggaccagcagctcctcggcatctggggctgttctggcaagctgatctgctgtactgccgtgccttggaacagctcctggtccaacaagtcccaagaggacatctgggacaacatgacctggatgcagtgggacagagagatcagcaactacaccaacaccatctaccgcctgctggaagatagccagacacagcaagagaaaaacgagaaggacctgctggccctggactgatgaggatcc

SEQ ID NO: 196 - DNA, 1980 bp, synthetic construct
  source 1..1980: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagaaatctgtgggtcacagtgtactacggcgtgcccgtgtggaaagaggcctgcaccacactgttttgtgccagcgacgccaaggcctacgatcccaaagtgcggaatgtgtgggccactcactgctgcgtgcccaccgatcctaatcctcaagagatgttcctggaaaacgtgaccgagaacttcaacatgtggaagaacgacatggtcgagcagatgcaccaggacatcatcagcctgtggtcccagagcctgaagccttgcgtgaagctgacccctctgtgcgtgaccctgaattgcagcgacgtgatcaacagcaccggcaccatgaagaactgcagcttcagcgtgaccaccgagctgcgggacagaaagcagaagaagcacgccctgttctaccggctggacatcgtgcccctggacgagaacgacaacagcagcaaggactacagactgatcaactgcaacaccagcacatgcacccaggcctgtcctaaagtgaccttcgatcccattcctatccactactgtgcccctgccggctacgccatcctgaagtgcaacaacaagaccttcaacggcacaggcccctgcaacaacgtgtccaccgtgcagtgtacccacggcatcaagccagtggtgtctacccagctgctgctgaatggctctctggccgaggaagaagtgatcatcagaagcgagaacctgaccgacaacgtcaagaccatcatcgtgcagctgaaagaacccgtgggcatcaattgcacccggcctaacaacaacatccggcagagcatcagaatcggccctggccagacttggtttgccaccggcgatatcatcggcgacatcagagaggcccactgcaatatcagcagcaccaagtggcgggaaaccctgcagcaagttggaggcaagctggccaagtacttcaacaacaaaacgatcaagttcacccagcactctggcggcgacctggaaatcaccacacacagcttcaactgcagaggcgagttcttctactgtaataccagccagctgttcaactccacctacaacgagacaggctccatcaatggcaccggcaatagcaccatcaagctgccctgccggatcaagcagatcatcaatatgtggcaaggcgtgggccagtgtatgtacgcccctcctatcgccggcaacatcatctgcagaagcaagatcaccggcctgctgctcacaagagatggcggcatcaacaagagcgaggaaatcttcagacccggcggaggcaacatgaaggacaattggagaagcgagctgtacaagtacaaggtggtggaaatcaagcccctgggcgtcgcccctacaaagtgcaagagaagagtggtggaacggcggagaaggcggagagctgttgttggactgggagccgtgttcctgggctttcttggagccgccggatctacaatgggagccgcctctatgaccctgaccgtgcaggctagacagctgctgagcggaattgtccagcagcagaacaactgcctgagagcccctgagtgccagcagtgtatgctgcagctgacagtgtggggaatcaagcagctgcaggccagagttctggccatcgagagatacctgaaggaccagcagctcctcggcatctggggctgttctggcaagctgatctgctgtactgccgtgccttggaacagctcctggtccaacaagtcccaagaggacatctgggacaacatgacctggatgcagtgggacagagagatcagcaactacaccaacaccatctaccgcctgctggaagatagccagacacagcaagagaaaaacgagaaggacctgctggccctggactgatgaggatcc

SEQ ID NO: 197 - DNA, 1941 bp, synthetic construct
  source 1..1941: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagaaatctgtgggtcacagtgtactacggcgtgcccgtgtggaaagaggccaagaccacactgttctgtgccagcgacgccaaggcctacgatcccgaagtgcacaatgtgtgggccactcacgcctgcgtgcccaccgatcctaatcctcaagagatgttcctggaaaacgtgaccgagaacttcaacatgtggaagaacgacatggtcgagcagatgcaccaggacatcatcagcctgtggtcccagagcctgaagccttgcgtgaagctgacccctctgtgcgtgaccctgaattgcagcgacgtgatcaacagcaccggcaccatgaagaactgcagcttcagcgtgaccaccgagctgcgggacagaaagcagaagaagcacgccctgttctaccggctggacatcgtgcccctggacgagaacgacaacagcagcaaggactacagactgatcaactgcaacaccagcacatgcacccaggcctgtcctaaagtgaccttcgatcccattcctatccactactgtgcccctgccggctacgccatcctgaagtgcaacaacaagaccttcaacggcacaggcccctgcaacaacgtgtccaccgtgcagtgtacccacggcatcaagccagtggtgtctacccagctgctgctgaatggctctctggccgaggaagaagtgatcatcagaagcgagaacctgaccgacaacgtcaagaccatcatcgtgcagctgaaagaacccgtgggcatcaattgcacccggcctaacaacaacatccggcagagcatcagaatcggccctggccagacctttttcgccaccggcgatatcatcggcgacatcagagaggcccactgcaatatcagcagcaccaagtggcgggaaaccctgcagcaagttggaggcaagctggccaagtacttcaacaacaaaacgatcaagttcacccagcactctggcggcgacctggaaatcaccacacacagcttcaactgcagaggcgagttcttctactgtaataccagccagctgttcaactccacctacaacgagacaggctccatcaatggcaccggcaatagcaccatcaagctgccctgccggatcaagcagatcatcaatatgtggcaaggcgtgggccagtgtatgtacgcccctcctatcgccggcaacatcatctgcagaagcaagatcaccggcctgctgctcacaagagatggcggcatcaacaagagcgaggaaatcttcagacccggcggaggcaacatgaaggacaattggagaagcgagctgtacaagtacaaggtggtggaaatcaagcccctgggcgtcgcccctacaaagtgcaagagaagagtggtggaacggcggagaaggcggagagctgttgttggactgggagccgtgttcctgggctttcttggagccgccggatctacaatgggagccgcctctatcaccctgaccgtgcaggctagacagctgctgagcggcaatcctgactggctgcctgatatgaccgtgtggggaatcaagcagctgcaggccagagttctggccatcgagagatacctgaaggaccagcagctcctcggcatctggggctgttctggcaagctgatctgctgtactgccgtgccttggaacagctcctggtccaacaagtcccaagaggacatctgggacaacatgacctggatgcagtgggacagagagatcagcaactacaccaacaccatctaccgcctgctggaagatagccagacacagcaagagaaaaacgagaaggacctgctggccctggactgatgaggatcc

SEQ ID NO: 198 - DNA, 1980 bp, synthetic construct
  source 1..1980: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagaaatctgtgggtcacagtgtactacggcgtgcccgtgtggaaggatgccgagacaacactgttctgtgccagcgacgccaaggcctacgatcccgagaaacacaacgtgtgggccactcacgcctgcgtgccaaccgatcctaatcctcaagagatgttcctggaaaacgtgaccgagaacttcaacatgtggaagaacgacatggtcgagcagatgcacaccgacatcatcagcctgtggtcccagagcctgaagccttgcgtgaagctgacccctctgtgcgtgaccctgaattgcagcgacgtgatcaacagcaccggcaccatgaagaactgcagcttcagcgtgaccaccgagctgcgggacagaaagcagaaagtgcacgccctgttctaccggctggacatcgttcctctggacgagaacgacaacagcagcaaggactacagactgatcaactgcaacaccagcacatgcacccaggcctgtcctaaagtgaccttcgatcccattcctatccactactgtgcccctgccggctacgccatcctgaagtgcaacaacaagaccttcaacggcacaggcccctgcaacaacgtgtccaccgtgcagtgtacccacggcatcaagccagtggtgtctacccagctgctgctgaatggctctctggccgaggaagaagtgatcatcagaagcgagaacctgaccgacaacgtcaagaccatcatcgtgcagctgaaagaacccgtgggcatcaattgcacccggcctaacaacaacatccggcagagcatcagaatcggccctggccagacctttttcgccaccggcgatatcatcggcgacatcagagaggcccactgcaatatcagcagcaccaagtggcgggaaaccctgcagcaagttggaggcaagctggccaagtacttcaacaacaaaacgatcaagttcacccagcactctggcggcgacctggaaatcaccacacacagcttcaactgcagaggcgagttcttctactgtaataccagccagctgttcaactccacctacaacgagacaggctccatcaatggcaccggcaatagcaccatcaagctgccctgccggatcaagcagatcatcaatatgtggcagcgcgtgggccagtgtatgtacgcccctccaatcgccggcaacatcatctgcagaagcaagatcaccggcctgctgctcacaagagatggcggcatcaacaagagcgaggaaatcttcagacccggcggaggcaacatgaaggacaattggagaagcgagctgtacaagtacaaggtggtggaaatcaagcccctgggcgtcgcccctacacggtgcaaaagaagagtggtcgagcggcggagaaggcggagagctgttgttggactgggagccgtgttcctgggctttcttggagccgccggatctacaatgggagccgcctctatcaccctgaccgtgcaggctagaaatctgctgagcggcatcgtccagcagcagagcaatctgctcagagcccctgaggcacagcaacacatgctgcagctgacagtgtggggaatcaagcagctgcaggccagagttctggccatcgagagatacctgagggaccagcagctcctcggcatctggggctgttctggcaagctgatctgctgtactgccgtgccttggaacagctcctggtccaacaagtcccaagaggacatctgggacaacatgacctggatgcagtgggacagagagatcagcaactacaccaacaccatctaccgcctgctggaagatagccagacacagcaagagaagaacgagaaggacctgctggccctggactgatgaggatcc

SEQ ID NO: 199 - DNA, 1971 bp, synthetic construct
  source 1..1971: mol_type = other DNA
gtcgacgccaccatgcctatgggatctctgcagcctctggccacactgtacctgctgggaatgctggtggctagcgtgctggccaagggaaagctgtgggtcacagtgtactacggcgtgcccgtgtggaaagaggccaagaccacactgttctgcgcctccgatgccagagcctacgagaaagaggtgcacaacgtctgggccacacacgcctgtgtgcctaccgatccatctcctcaagagctgttcctgaagaacgtgaccgagaacttcaacatgtggaagaacgacatggtggaccagatgcacgaggacatcatcagcctgtgggaccagagcctgaagccttgcgtgaagctgacccctctgtgcgtgaccctgatctgcagcaatgccaccgtgaagaaccggaccgtggaagagatgaagaactgcagcttcaacaccaccaccgagatccgggacaaagagaagaaagagtacgccctgttctacaagcccgacgtggtgcccctggacgagacaaacaataccagcgagtaccggctgatcaactgcaacaccagcgcctgcactcaggcctgtcctaaagtgaccttcgagcccattcctatccactactgtgcccctgccggctacgccatcctgaagtgcaacaacgagacattcaacggcacaggcccctgcagcaacgtgtccacagtgcagtgtacccacggcatcagacccgtggtgtctacacagctgctgctgaatggaagcctggccgagaaagaaatcgtgatcagaagcgagaacctgaccaacaacgccaagatcatcatcgtgcacctgaacacccctgtggaaatcgtgtgcacccggcctaacaacaacaccagaaagagcgtgcggatcggccctggccagaccttttatgccaccggcgatatcatcggcgacatcagacaggcccactgtaacatcagcgagaagcagtggaacgacaccctgcagaaagtgggcaaagagctgaagaagcacttccccaacaagaccattaagtacgcccagagcgctggcggcgacatggaaatcaccacacacagcttcaattgtggcggcgagttcttctactgcaataccgccaagctgttcaacggaacctacaacggcaccgacatcagcaccaacagcagcgccgacagcaacagcaccatcactctgcagtgccggatcaagcagatcatcaatatgtggcaaggcgtcggccggtgtatgtacgcccctcctatcgccggcaacatcacatgcaagagccacatcacaggcctgctgctcacaagagatggcggcacaaacagctccggcaaagaggaaatcttccggcctgccggcggagacatgcgggataattggagaagcgagctgtacaagtacaaggtggtggaaatccagccactgggaatcgccccaacaggctgcaagagaagagtggtcgagcggagaagaaggcggagagctgctggactgggtgccctgtttctgggctttcttggagccgccggaagcacaatgggagccgcctctattaccctgaccgtgcaggctagacagctgctctctggaatcgtgcagcagcagagcaacctgctgagagcccctgaagctcagcagcacatgctgcagctgacagtgtggggcatcaagcagctgcaggcaagagtgctggccctggaaagatacctgaaggatcagcagctcctcggcatgtggggctgttctggcaagctgatctgctgcaccaacgtgccctggaacacctcttggagcaacaagagcgagacagacatctgggacaacatgacctggatgcagtgggagagagagatcagcaactacaccgagacaatctacaaactgctcgaggacagccagaaccagcaagagagaaacgagcaggacctgctggctctggactgatgaggatcc

SEQ ID NO: 200 - DNA, 2004 bp, synthetic construct
  source 1..2004: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagaaagggaaagctgtgggtcacagtgtactacggcgtgcccgtgtggaaagaggccaagaccacactgttctgcgcctccgatgccagagcctacgagaaagaggtgcacaacgtctgggccacacacgcctgtgtgcctaccgatccatctcctcaagagctgttcctgaagaacgtgaccgagaacttcaacatgtggaagaacgacatggtggaccagatgcacgaggacatcatcagcctgtgggaccagagcctgaagccttgcgtgaagctgacccctctgtgcgtgaccctgatctgcagcaatgccaccgtgaagaaccggaccgtggaagagatgaagaactgcagcttcaacaccaccaccgagatccgggacaaagagaagaaagagtacgccctgttctacaagcccgacgtggtgcccctggacgagacaaacaataccagcgagtaccggctgatcaactgcaacaccagcgcctgcactcaggcctgtcctaaagtgaccttcgagcccattcctatccactactgtgcccctgccggctacgccatcctgaagtgcaacaacgagacattcaacggcacaggcccctgcagcaacgtgtccacagtgcagtgtacccacggcatcagacccgtggtgtctacacagctgctgctgaatggaagcctggccgagaaagaaatcgtgatcagaagcgagaacctgaccaacaacgccaagatcatcatcgtgcacctgaacacccctgtggaaatcgtgtgcacccggcctaacaacaacaccagaaagagcgtgcggatcggccctggccagaccttttatgccaccggcgatatcatcggcgacatcagacaggcccactgtaacatcagcgagaagcagtggaacgacaccctgcagaaagtgggcaaagagctgaagaagcacttccccaacaagaccattaagtacgcccagagcgctggcggcgacatggaaatcaccacacacagcttcaattgtggcggcgagttcttctactgcaataccgccaagctgttcaacggaacctacaacggcaccgacatcagcaccaacagcagcgccgacagcaacagcaccatcactctgcagtgccggatcaagcagatcatcaatatgtggcaaggcgtcggccggtgtatgtacgcccctcctatcgccggcaacatcacatgcaagagccacatcacaggcctgctgctcacaagagatggcggcacaaacagctccggcaaagaggaaatcttccggcctgccggcggagacatgcgggataattggagaagcgagctgtacaagtacaaggtggtggaaatccagccactgggaatcgccccaacaggctgcaagagaagagtggtcgagcggagaagaaggcggagagctgctggactgggtgccctgtttctgggctttcttggagccgccggaagcacaatgggagccgcctctattaccctgaccgtgcaggctagacagctgctctctggaatcgtgcagcagcagagcaacctgctgagagcccctgaagctcagcagcacatgctgcagctgacagtgtggggcatcaagcagctgcaggcaagagtgctggccctggaaagatacctgaaggatcagcagctcctcggcatgtggggctgttctggcaagctgatctgctgcaccaacgtgccctggaacacctcttggagcaacaagagcgagacagacatctgggacaacatgacctggatgcagtgggagagagagatcagcaactacaccgagacaatctacaaactgctcgaggacagccagaaccagcaagagagaaacgagcaggacctgctggctctggactgatgaggatcc

SEQ ID NO: 201 - DNA, 1971 bp, synthetic construct
  source 1..1971: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctgttcctgaagaacgtgaccgagaacttcaacatgtggaagaacgacatggtggaccagatgcacgaggacatcatcagcctgtgggaccagagcctgaagccttgcgtgaagctgacccctctgtgcgtgaccctgatctgcagcaatgccaccgtgaagaaccggaccgtggaagagatgaagaactgcagcttcaacaccaccaccgagatccgggacaaagagaagaaagagtacgccctgttctacaagcccgacgtggtgcccctggacgagacaaacaataccagcgagtaccggctgatcaactgcaacaccagcgcctgcactcaggcctgtcctaaagtgaccttcgagcccattcctatccactactgtgcccctgccggctacgccatcctgaagtgcaacaacgagacattcaacggcacaggcccctgcagcaacgtgtccacagtgcagtgtacccacggcatcagacccgtggtgtctacacagctgctgctgaatggaagcctggccgagaaagaaatcgtgatcagaagcgagaacctgaccaacaacgccaagatcatcatcgtgcacctgaacacccctgtggaaatcgtgtgcacccggcctaacaacaacaccagaaagagcgtgcggatcggccctggccagaccttttatgccaccggcgatatcatcggcgacatcagacaggcccactgtaacatcagcgagaagcagtggaacgacaccctgcagaaagtgggcaaagagctgaagaagcacttccccaacaagaccattaagtacgcccagagcgctggcggcgacatggaaatcaccacacacagcttcaattgtggcggcgagttcttctactgcaataccgccaagctgttcaacggaacctacaacggcaccgacatcagcaccaacagcagcgccgacagcaacagcaccatcactctgcagtgccggatcaagcagatcatcaatatgtggcaaggcgtcggccggtgtatgtacgcccctcctatcgccggcaacatcacatgcaagagccacatcacaggcctgctgctcacaagagatggcggcacaaacagctccggcaaagaggaaatcttccggcctgccggcggagacatgcgggataattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagcagctggtccaacagaaacctgagcgagatctgggataacatgacctggctgcagtgggacaaagaaatcagcaactacacccagatcatctacggcctgctggaagagagccagaaccagcaagagaaaaacgagcaggacctgctggccctggactgataaggatcc

SEQ ID NO: 202 - DNA, 2013 bp, synthetic construct
  source 1..2013: mol_type = other DNA
gtcgacgccaccatgcctatgggatctctgcagcctctggccacactgtacctgctgggaatgctggtggcttctgtgctggccgagggcaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctgaagaacgtgaccgagaacttcaacatgtggaagaacgacatggtggaccagatgcacgaggacatcatcagcctgtgggaccagagcctgaagccttgcgtgaagctgacccctctgtgcgtgaccctgatctgcagcaacgccatcgtgaagaacagcaccaccgagaagatgagcaatgtgaccgtgaacaacatcaccattgaggaaatgaagaactgcagcttcaacaccaccacagagatccgggacaagattaagaaagagcgggccctgttctacagacccgacatcgtgcccctgaacgacgagacaaacaacaccagcaagtaccggctgatcaactgcaacacctccgcctgcactcaggcctgtcctaaagtgaccttcgagcccattcctatccactactgtgcccctgccggctatgccatcctgaagtgcaacgacgaaaccttcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacgcccctgtggaaatcgtgtgcacccggccttacaacaacacccggaagtctgtgcggatcggccctggccagacattctatgccaccggcgatatcatcggcgaccctagacaggcccactgtaacatcagcaaagagatctggaacaagaccctgcaagaagtgggcaaagagctgcagaagcacttccccaacaagaccatccggtacaaccagtctgccggcggagacatggaaatcaccacacacagcttcaattgtggcggcgagttcttctactgcaataccagcaacctgttcaacagcacctacaacgacacctacatcagccccaactccaccaatagcaccagcatcatcaccctgcagtgcaagatcaagcagatcatcaatatgtggcaaggcgtcggccggtgtatgtacgcccctcctatcgccggcaacatcacatgcaagagcaatatcaccggcctgctgctcaccagagatggcggcattaacaacgtgtccaacgagacagagacattcagacccgctggcggcgacatgcgggacaattggagaagcgagctgtacaagtacaaggtggtggaagtgcagcccctgggaatcgctcctaccggctgcaagagaagagtggtggaaagacgcagacggcggagagctgttggactgggtgctctgttcctgggctttctgggagccgccggatctacaatgggagctgccagcatcacactgaccgtgcaggctagacagctgctgagcggaattgtgcagcagcagagcaacctgctgagagcccctgaagcacagcagcacatgctgcagctgacagtgtggggcatcaaacagctgcaggccagagtgctggccctggaaagatacctgaaggaccagcagctcctcggaatctggggctgttctggcaagctgatctgctgcaccaacgtgccctggaacacctcctggtccaacaagagcgagacagacatttggaacaacatgacctggatgcagtgggagagagagatcagcaactacaccggcaccatctacaagctgctggaagatagccagaaccagcaagagcggaacgagaaggacctgctggctctggactgatgaggatcc

SEQ ID NO: 203 - DNA, 2046 bp, synthetic construct
  source 1..2046: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagagagggcaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctgaagaacgtgaccgagaacttcaacatgtggaagaacgacatggtggaccagatgcacgaggacatcatcagcctgtgggaccagagcctgaagccttgcgtgaagctgacccctctgtgcgtgaccctgatctgcagcaacgccatcgtgaagaacagcaccaccgagaagatgagcaatgtgaccgtgaacaacatcaccattgaggaaatgaagaactgcagcttcaacaccaccacagagatccgggacaagattaagaaagagcgggccctgttctacagacccgacatcgtgcccctgaacgacgagacaaacaacaccagcaagtaccggctgatcaactgcaacacctccgcctgcactcaggcctgtcctaaagtgaccttcgagcccattcctatccactactgtgcccctgccggctatgccatcctgaagtgcaacgacgaaaccttcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacgcccctgtggaaatcgtgtgcacccggccttacaacaacacccggaagtctgtgcggatcggccctggccagacattctatgccaccggcgatatcatcggcgaccctagacaggcccactgtaacatcagcaaagagatctggaacaagaccctgcaagaagtgggcaaagagctgcagaagcacttccccaacaagaccatccggtacaaccagtctgccggcggagacatggaaatcaccacacacagcttcaattgtggcggcgagttcttctactgcaataccagcaacctgttcaacagcacctacaacgacacctacatcagccccaactccaccaatagcaccagcatcatcaccctgcagtgcaagatcaagcagatcatcaatatgtggcaaggcgtcggccggtgtatgtacgcccctcctatcgccggcaacatcacatgcaagagcaatatcaccggcctgctgctcaccagagatggcggcattaacaacgtgtccaacgagacagagacattcagacccgctggcggcgacatgcgggacaattggagaagcgagctgtacaagtacaaggtggtggaagtgcagcccctgggaatcgctcctaccggctgcaagagaagagtggtggaaagacgcagacggcggagagctgttggactgggtgctctgttcctgggctttctgggagccgccggatctacaatgggagctgccagcatcacactgaccgtgcaggctagacagctgctgagcggaattgtgcagcagcagagcaacctgctgagagcccctgaagcacagcagcacatgctgcagctgacagtgtggggcatcaaacagctgcaggccagagtgctggccctggaaagatacctgaaggaccagcagctcctcggaatctggggctgttctggcaagctgatctgctgcaccaacgtgccctggaacacctcctggtccaacaagagcgagacagacatttggaacaacatgacctggatgcagtgggagagagagatcagcaactacaccggcaccatctacaagctgctggaagatagccagaaccagcaagagcggaacgagaaggacctgctggctctggactgatgaggatcc

SEQ ID NO: 204 - DNA, 2013 bp, synthetic construct
  source 1..2013: mol_type = other DNA
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctgaagaacgtgaccgagaacttcaacatgtggaagaacgacatggtggaccagatgcacgaggacatcatcagcctgtgggaccagagcctgaagccttgcgtgaagctgacccctctgtgcgtgaccctgatctgcagcaacgccatcgtgaagaacagcaccaccgagaagatgagcaatgtgaccgtgaacaacatcaccattgaggaaatgaagaactgcagcttcaacaccaccacagagatccgggacaagattaagaaagagcgggccctgttctacagacccgacatcgtgcccctgaacgacgagacaaacaacaccagcaagtaccggctgatcaactgcaacacctccgcctgcactcaggcctgtcctaaagtgaccttcgagcccattcctatccactactgtgcccctgccggctatgccatcctgaagtgcaacgacgaaaccttcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacgcccctgtggaaatcgtgtgcacccggccttacaacaacacccggaagtctgtgcggatcggccctggccagacattctatgccaccggcgatatcatcggcgaccctagacaggcccactgtaacatcagcaaagagatctggaacaagaccctgcaagaagtgggcaaagagctgcagaagcacttccccaacaagaccatccggtacaaccagtctgccggcggagacatggaaatcaccacacacagcttcaattgtggcggcgagttcttctactgcaataccagcaacctgttcaacagcacctacaacgacacctacatcagccccaactccaccaatagcaccagcatcatcaccctgcagtgcaagatcaagcagatcatcaatatgtggcaaggcgtcggccggtgtatgtacgcccctcctatcgccggcaacatcacatgcaagagcaatatcaccggcctgctgctcaccagagatggcggcattaacaacgtgtccaacgagacagagacattcagacccgctggcggcgacatgcgggacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgacactgaccgtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaactccagctggtccaacagaaacctgagcgagatctgggataacatgacctggctgcagtgggacaaagaaatcagcaactacacccagatcatctacggactgctggaagagagccagaaccagcaagagaaaaacgagcaggacctgctggccctggactgataaggatcc

SEQ ID NO: 205 - DNA, 1974 bp, synthetic construct
  source 1..1974: mol_type = other DNA
gtcgacgccaccatgcctatgggatctctgcagcctctggccacactgtacctgctgggaatgctggtggcttctgtgctggccgagggcaatctgtgggtcacagtgtactatggcgtgcccgtgtggaaagaggccaagaccacactgttctgtgccagcgacgccaaggcctacgtgaaagaggtgcacaacgtctgggccacacacgcctgtgtgcctaccgatccatctcctcaagagctgttcctgaagaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagcaagttcgaggaaatgaagaactgcagcttcaacaccaccaccgagatccgggacaaagagaagaaagagtacgccctgttctacaggcccgacatcgtgcccctggacaacgagacaagcaacatcagcgagtaccggctgatcaactgcaacaccagcgcctgcactcaggcctgtcctaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacaccagaaagagcgtgcggatcggccctggccagaccttttatgccaccggcgacatcatcggcaaccccagacaggcccactgtaacatcagcaaagagcggtggaacgacaccctgcagaaagtgggcaaagagctgcagaagcacttccccaacaagaccatcaagtacaaccagagcgctggcggcgacatggaaatcaccacacacagcttcaattgtggcggcgagttcttctactgcaatacctccaacctgttcaacagcacctacaacgacacctacatcagcaccaactccaccaatagcaccagctacattaccctgcagtgccggatcaagcagatcatcaatatgtggcaaggcgtcggccggtgtatgtacgcccctcctatcgccggcaacatcacatgcaagtccaacatcaccggcctgctgctcacaagagatggcggcatcaacaaggacagcaacgaaaccgaaaccttccggcctgccggcggagacatgagagacaattggagaagcgagctgtacaagtacaaggtggtggaagtgcagcccctgggaatcgctcctaccggctgcaagagaagagtggtggaaagacgcagacggcggagagctgctggactgggtgctctgtttctgggctttctgggagccgccggatctacaatgggagctgccagcatcaccctgaccgtgcaggctagacaactgctgtctggcatcgtgcagcagcagagcaacctgctgagagcccctgaagctcagcagcacatgctgcagctgacagtgtggggcatcaaacagctgcaggccagagttctggccctggaaagatacctgaaggatcagcagctcctcggcatgtggggctgttctggcaagctgatctgctgcaccaacgtgccctggaacacctcctggtccaacaagagcgagacagacatctgggacaacatgacctggatgcagtgggagagagagatcagcaactacaccggcaccatctacaagctgctcgaggacagccagaaccagcaagagagaaacgagcaggacctgctggctctggactgatgaggatcc

SEQ ID NO: 206 - DNA, 2007 bp, synthetic construct
  source 1..2007: mol_type = other DNA
gtcgacgccaccatggatgctatgaagaggggcctgtgctgcgtgctgcttctgtgtggcgctgtgtttgtgtcccctagccaagagatccacgccagattcagaagaggcgccagagagggcaatctgtgggtcacagtgtactatggcgtgcccgtgtggaaagaggccaagaccacactgttctgtgccagcgacgccaaggcctacgtgaaagaggtgcacaacgtctgggccacacacgcctgtgtgcctaccgatccatctcctcaagagctgttcctgaagaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagcaagttcgaggaaatgaagaactgcagcttcaacaccaccaccgagatccgggacaaagagaagaaagagtacgccctgttctacaggcccgacatcgtgcccctggacaacgagacaagcaacatcagcgagtaccggctgatcaactgcaacaccagcgcctgcactcaggcctgtcctaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacaccagaaagagcgtgcggatcggccctggccagaccttttatgccaccggcgacatcatcggcaaccccagacaggcccactgtaacatcagcaaagagcggtggaacgacaccctgcagaaagtgggcaaagagctgcagaagcacttccccaacaagaccatcaagtacaaccagagcgctggcggcgacatggaaatcaccacacacagcttcaattgtggcggcgagttcttctactgcaatacctccaacctgttcaacagcacctacaacgacacctacatcagcaccaactccaccaatagcaccagctacattaccctgcagtgccggatcaagcagatcatcaatatgtggcaaggcgtcggccggtgtatgtacgcccctcctatcgccggcaacatcacatgcaagtccaacatcaccggcctgctgctcacaagagatggcggcatcaacaaggacagcaacgaaaccgaaaccttccggcctgccggcggagacatgagagacaattggagaagcgagctgtacaagtacaaggtggtggaagtgcagcccctgggaatcgctcctaccggctgcaagagaagagtggtggaaagacgcagacggcggagagctgctggactgggtgctctgtttctgggctttctgggagccgccggatctacaatgggagctgccagcatcaccctgaccgtgcaggctagacaactgctgtctggcatcgtgcagcagcagagcaacctgctgagagcccctgaagctcagcagcacatgctgcagctgacagtgtggggcatcaaacagctgcaggccagagttctggccctggaaagatacctgaaggatcagcagctcctcggcatgtggggctgttctggcaagctgatctgctgcaccaacgtgccctggaacacctcctggtccaacaagagcgagacagacatctgggacaacatgacctggatgcagtgggagagagagatcagcaactacaccggcaccatctacaagctgctcgaggacagccagaaccagcaagagagaaacgagcaggacctgctggctctggactgatgaggatcc

SEQ ID NO: 207 - DNA, 1974 bp, synthetic construct
  source 1..1974: mol_type = other DNA
gtcgacgccaccatgcctatgggatctctgcagcctctggccacactgtacctgctgggaatgctggtggcttctgtgctggccgccgagaatctggtcacagtgtactatggcgtgcccgtgtggaaagaggccaagaccacactgttctgtgccagcgacgccaaggcctacgtgaaagaggtgcacaacgtctgggccacacacgcctgtgtgcctaccgatccatctcctcaagagctgttcctgaagaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagcaagttcgaggaaatgaagaactgcagcttcaacaccaccaccgagatccgggacaaagagaagaaagagtacgccctgttctacaggcccgacatcgtgcccctggacaacgagacaagcaacatcagcgagtaccggctgatcaactgcaacaccagcgcctgcactcaggcctgtcctaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacaccagaaagagcgtgcggatcggccctggccagaccttttatgccaccggcgacatcatcggcaaccccagacaggcccactgtaacatcagcaaagagcggtggaacgacaccctgcagaaagtgggcaaagagctgcagaagcacttccccaacaagaccatcaagtacaaccagagcgctggcggcgacatggaaatcaccacacacagcttcaattgtggcggcgagttcttctactgcaatacctccaacctgttcaacagcacctacaacgacacctacatcagcaccaactccaccaatagcaccagctacattaccctgcagtgccggatcaagcagatcatcaatatgtggcaaggcgtcggccggtgtatgtacgcccctcctatcgccggcaacatcacatgcaagtccaacatcaccggcctgctgctcacaagagatggcggcatcaacaaggacagcaacgaaaccgaaaccttccggcctgccggcggagacatgagagacaattggagaagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaactccagctggtccaacagaaacctgagcgagatctgggataacatgacctggctgcagtgggacaaagaaatcagcaactacacccagatcatctacggactgctggaagagagccagaaccagcaagagaaaaacgagcaggacctgctggccctggactgataaggatcc

SEQ ID NO: 208 - protein, 655 aa, synthetic construct
MPMGSLQPLATLYLLGMLVASVLAAENLWVTVYYGVPVWEDADTPLFCASDAKAYSTESHNVWATHACVPTDPSPQEISLDNVTENFNMWKNNMVEQMHEDIISLWDESLKPCVKLTPLCVTLNCTNVNNSSATNNSMVDDREGLKNCSFNITTELRDKKKQEHALFYRLDIVPINGNSNSNSSVGDYRLINCNVSTCKQACPKMSFDPIPIHYCAPAGFAILKCRDKKFNGTGSCKNVSTVQCTHGIKPVISTQLLLNGSVAEEEIMIRSENFTNNAKNIIVQFNKTIDIMCTRPNNNTRKSISLGPGQAIYATGDIIGNIRQAHCNISGADWGNMIRNVSEKLKEIFNKTTITFKASAGGDLEITTHSFNCRGEFFYCDTSDLFNSSRFNNSSNDTNDTITLPCKIKQIVRMWQRVGQCMYAPPIAGNITCRSNITGLLLTRDGGGNNTNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 209 - protein, 655 aa, synthetic construct
MPMGSLQPLATLYLLGMLVASVLASNNLWVTVYYGVPVWEDADTPLFCASDAKAYSTESHNVWATHACVPTDPSPQEISLDNVTENFNMWKNNMVEQMHEDIISLWDESLKPCVKLTPLCVTLNCTNVNNSSATNNSMVDDREGLKNCSFNITTELRDKKKQEHALFYRLDIVPINGNSNSNSSVGDYRLINCNVSTIKQACPKMSFDPIPIHYCAPAGFAILKCRDKKFNGTGSCKNVSTVQCTHGIKPVISTQLLLNGSVAEEEIMIRSENFTNNAKNIIVQFNKTIDIMCTRPNNNTRKSISLGPGQAIYATGDIIGNIRQAHCNISGADWGNMIRNVSEKLKEIFNKTTITFKASAGGDLEITTHSFNCRGEFFYCDTSDLFNSSRFNNSSNDTNDTITLPCKIKQIVRMWQRVGQAMYAPPIAGNITCRSNITGLLLTRDGGGNNTNETETFRPAGGDMRDNWRSELYKYKVVKINPLGVAPTRCRRRVVGRRRRRRAIGMGAVLLGFLGTAGSTMGAASITLTVQVRQLLSGIVQQQSNLLRAPEAQQHLLQLTVWGIKQLQARILAVERYLKDQQLLGIWGCSGKLICCTNVPWNASWSNRSYNDIWENLTWVEWEREISNYTQQIYSLLEESQNQQEKNEQDLLALD

SEQ ID NO: 210 - protein, 655 aa, synthetic construct
MPMGSLQPLATLYLLGMLVASVLASNNLWVTVYYGVPVWEDADTPLFCASDAKAYSTESHNVWATHACVPTDPSPQEISLDNVTENFNMWKNNMVEQMHEDIISLWDESLKPCVKLTPLCVTLNCTNVNNSSATNNSMVDDREGLKNCSFNITTELRDKKKQEHALFYRLDIVPINGNSNSNSSVGDYRLINCNVSTCKQACPKMSFDPIPIHYCAPAGFAILKCRDKKFNGTGSCKNVSTVQCTHGIKPVISTQLLLNGSVAEEEIMIRSENFTNNAKNIIVQFNKTIDIMCTRPNNNTRKSISLGPGQAIYATGDIIGNIRQAHCNISGADWGNMIRNVSEKLKEIFNKTTITFKASAGGDLEITTHSFNCRGEFFYCDTSDLFNSSRFNNSSNDTNDTITLPCKIKQIVRMWQRVGQCMYAPPIAGNITCRSNITGLLLTRDGGGNNTNETETFRPAGGDMRDNWRSELYKYKVVKINPLGVAPTRCRRRVVGRRRRRRAIGMGAVLLGFLGTAGSTMGAASITLTVQVRQLLSGIVQQQSNLLRAPEAQQHLLQLTVWGIKQLQARILAVERYLKDQQLLGIWGCSGKLICCTNVPWNASWSNRSYNDIWENLTWVEWEREISNYTQQIYSLLEESQNQQEKNEQDLLALD

SEQ ID NO: 211 - protein, 642 aa, synthetic construct
MPMGSLQPLATLYLLGMLVASVLASNNLWVTVYYGVPVWEDADTPLFCASDAKAYSTESHNVWATHACVPTDPSPQEISLDNVTENFNMWKNNMVEQMHEDIISLWDESLKPCVKLTPLCVTLNCTNVNNSSATNNSMVDDREGLKNCSFNITTELRDKKKQEHALFYRLDIVPINGNSNSNSSVGDYRLINCNVSTCKQACPKMSFDPIPIHYCAPAGFAILKCRDKKFNGTGSCKNVSTVQCTHGIKPVISTQLLLNGSVAEEEIMIRSENFTNNAKNIIVQFNKTIDIMCTRPNNNTRKSISLGPGQAIYATGDIIGNIRQAHCNISGADWGNMIRNVSEKLKEIFNKTTITFKASAGGDLEITTHSFNCRGEFFYCDTSDLFNSSRFNNSSNDTNDTITLPCKIKQIVRMWQRVGQCMYAPPIAGNITCRSNITGLLLTRDGGGNNTNETETFRPAGGDMRDNWRSELYKYKVVKINPLGVAPTRCRRRVVGRRRRRRAIGMGAVLLGFLGTAGSTMGAASITLTVQVRQLLSGNPDWLPDMTVWGIKQLQARILAVERYLKDQQLLGIWGCSGKLICCTNVPWNASWSNRSYNDIWENLTWVEWEREISNYTQQIYSLLEESQNQQEKNEQDLLALD

SEQ ID NO: 212 - protein, 655 aa, synthetic construct
MPMGSLQPLATLYLLGMLVASVLASNNLWVTVYYGVPVWEDACTPLFCASDAKAYSTKSRNVWATCCCVPTDPSPQEISLDNVTENFNMWKNNMVEQMHEDIISLWDESLKPCVKLTPLCVTLNCTNVNNSSATNNSMVDDREGLKNCSFNITTELRDKKKQEHALFYRLDIVPINGNSNSNSSVGDYRLINCNVSTIKQACPKMSFDPIPIHYCAPAGFAILKCRDKKFNGTGSCKNVSTVQCTHGIKPVISTQLLLNGSVAEEEIMIRSENFTNNAKNIIVQFNKTIDIMCTRPNNNTRKSISLGPGQAWYATGDIIGNIRQAHCNISGADWGNMIRNVSEKLKEIFNKTTITFKASAGGDLEITTHSFNCRGEFFYCDTSDLFNSSRFNNSSNDTNDTITLPCKIKQIVRMWQRVGQAMYAPPIAGNITCRSNITGLLLTRDGGGNNTNETETFRPAGGDMRDNWRSELYKYKVVKINPLGVAPTRCRRRVVGRRRRRRAIGMGAVLLGFLGTAGSTMGAASMTLTVQVRQLLSGIVQQQSNCLRAPECQQCLLQLTVWGIKQLQARILAVERYLKDQQLLGIWGCSGKLICCTNVPWNASWSNRSYNDIWENLTWVEWEREISNYTQQIYSLLEESQNQQEKNEQDLLALD

SEQ ID NO: 213 - protein, 655 aa, synthetic construct
MPMGSLQPLATLYLLGMLVASVLASNNLWVTVYYGVPVWEDAETPLFCASDAKAYSTEKHNVWATHACVPTDPSPQEISLDNVTENFNMWKNNMVEQMHTDIISLWDESLKPCVKLTPLCVTLNCTNVNNSSATNNSMVDDREGLKNCSFNITTELRDKKKQVHALFYRLDIVPINGNSNSNSSVGDYRLINCNVSTIKQACPKMSFDPIPIHYCAPAGFAILKCRDKKFNGTGSCKNVSTVQCTHGIKPVISTQLLLNGSVAEEEIMIRSENFTNNAKNIIVQFNKTIDIMCTRPNNNTRKSIRLGPGQAIYATGDIIGNIRQAHCNISGADWGNMIRNVSEKLKEIFNKTTITFKASAGGDLEITTHSFNCRGEFFYCDTSDLFNSSRFNNSSNDTNDTITLPCKIKQIVRMWQRVGQAMYAPPIAGNITCRSNITGLLLTRDGGGNNTNETETFRPAGGDMRDNWRSELYKYKVVKINPLGVAPTRCRRRVVGRRRRRRAIGMGAVLLGFLGTAGSTMGAASITLTVQVRNLLSGIVQQQSNLLRAPEAQQHLLQLTVWGIKQLQARILAVERYLRDQQLLGIWGCSGKLICCTNVPWNASWSNRSYNDIWENLTWVEWEREISNYTQQIYSLLEESQNQQEKNEQDLLALD

SEQ ID NO: 214 - protein, 666 aa, synthetic construct
MDAMKRGLCCVLLLCGAVFVSPSQEIHARFRRGARAENLWVTVYYGVPVWEDADTPLFCASDAKAYSTESHNVWATHACVPTDPSPQEISLDNVTENFNMWKNNMVEQMHEDIISLWDESLKPCVKLTPLCVTLNCTNVNNSSATNNSMVDDREGLKNCSFNITTELRDKKKQEHALFYRLDIVPINGNSNSNSSVGDYRLINCNVSTCKQACPKMSFDPIPIHYCAPAGFAILKCRDKKFNGTGSCKNVSTVQCTHGIKPVISTQLLLNGSVAEEEIMIRSENFTNNAKNIIVQFNKTIDIMCTRPNNNTRKSISLGPGQAIYATGDIIGNIRQAHCNISGADWGNMIRNVSEKLKEIFNKTTITFKASAGGDLEITTHSFNCRGEFFYCDTSDLFNSSRFNNSSNDTNDTITLPCKIKQIVRMWQRVGQCMYAPPIAGNITCRSNITGLLLTRDGGGNNTNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 215 - protein, 666 aa, synthetic construct
MDAMKRGLCCVLLLCGAVFVSPSQEIHARFRRGARSNNLWVTVYYGVPVWEDADTPLFCASDAKAYSTESHNVWATHACVPTDPSPQEISLDNVTENFNMWKNNMVEQMHEDIISLWDESLKPCVKLTPLCVTLNCTNVNNSSATNNSMVDDREGLKNCSFNITTELRDKKKQEHALFYRLDIVPINGNSNSNSSVGDYRLINCNVSTIKQACPKMSFDPIPIHYCAPAGFAILKCRDKKFNGTGSCKNVSTVQCTHGIKPVISTQLLLNGSVAEEEIMIRSENFTNNAKNIIVQFNKTIDIMCTRPNNNTRKSISLGPGQAIYATGDIIGNIRQAHCNISGADWGNMIRNVSEKLKEIFNKTTITFKASAGGDLEITTHSFNCRGEFFYCDTSDLFNSSRFNNSSNDTNDTITLPCKIKQIVRMWQRVGQAMYAPPIAGNITCRSNITGLLLTRDGGGNNTNETETFRPAGGDMRDNWRSELYKYKVVKINPLGVAPTRCRRRVVGRRRRRRAIGMGAVLLGFLGTAGSTMGAASITLTVQVRQLLSGIVQQQSNLLRAPEAQQHLLQLTVWGIKQLQARILAVERYLKDQQLLGIWGCSGKLICCTNVPWNASWSNRSYNDIWENLTWVEWEREISNYTQQIYSLLEESQNQQEKNEQDLLALD

SEQ ID NO: 216 - protein, 666 aa, synthetic construct
MDAMKRGLCCVLLLCGAVFVSPSQEIHARFRRGARSNNLWVTVYYGVPVWEDADTPLFCASDAKAYSTESHNVWATHACVPTDPSPQEISLDNVTENFNMWKNNMVEQMHEDIISLWDESLKPCVKLTPLCVTLNCTNVNNSSATNNSMVDDREGLKNCSFNITTELRDKKKQEHALFYRLDIVPINGNSNSNSSVGDYRLINCNVSTCKQACPKMSFDPIPIHYCAPAGFAILKCRDKKFNGTGSCKNVSTVQCTHGIKPVISTQLLLNGSVAEEEIMIRSENFTNNAKNIIVQFNKTIDIMCTRPNNNTRKSISLGPGQAIYATGDIIGNIRQAHCNISGADWGNMIRNVSEKLKEIFNKTTITFKASAGGDLEITTHSFNCRGEFFYCDTSDLFNSSRFNNSSNDTNDTITLPCKIKQIVRMWQRVGQCMYAPPIAGNITCRSNITGLLLTRDGGGNNTNETETFRPAGGDMRDNWRSELYKYKVVKINPLGVAPTRCRRRVVGRRRRRRAIGMGAVLLGFLGTAGSTMGAASITLTVQVRQLLSGIVQQQSNLLRAPEAQQHLLQLTVWGIKQLQARILAVERYLKDQQLLGIWGCSGKLICCTNVPWNASWSNRSYNDIWENLTWVEWEREISNYTQQIYSLLEESQNQQEKNEQDLLALD

SEQ ID NO: 217 - protein, 653 aa, synthetic construct
MDAMKRGLCCVLLLCGAVFVSPSQEIHARFRRGARSNNLWVTVYYGVPVWEDADTPLFCASDAKAYSTESHNVWATHACVPTDPSPQEISLDNVTENFNMWKNNMVEQMHEDIISLWDESLKPCVKLTPLCVTLNCTNVNNSSATNNSMVDDREGLKNCSFNITTELRDKKKQEHALFYRLDIVPINGNSNSNSSVGDYRLINCNVSTCKQACPKMSFDPIPIHYCAPAGFAILKCRDKKFNGTGSCKNVSTVQCTHGIKPVISTQLLLNGSVAEEEIMIRSENFTNNAKNIIVQFNKTIDIMCTRPNNNTRKSISLGPGQAIYATGDIIGNIRQAHCNISGADWGNMIRNVSEKLKEIFNKTTITFKASAGGDLEITTHSFNCRGEFFYCDTSDLFNSSRFNNSSNDTNDTITLPCKIKQIVRMWQRVGQCMYAPPIAGNITCRSNITGLLLTRDGGGNNTNETETFRPAGGDMRDNWRSELYKYKVVKINPLGVAPTRCRRRVVGRRRRRRAIGMGAVLLGFLGTAGSTMGAASITLTVQVRQLLSGNPDWLPDMTVWGIKQLQARILAVERYLKDQQLLGIWGCSGKLICCTNVPWNASWSNRSYNDIWENLTWVEWEREISNYTQQIYSLLEESQNQQEKNEQDLLALD

SEQ ID NO: 218 - protein, 666 aa, synthetic construct
MDAMKRGLCCVLLLCGAVFVSPSQEIHARFRRGARSNNLWVTVYYGVPVWEDACTPLFCASDAKAYSTKSRNVWATCCCVPTDPSPQEISLDNVTENFNMWKNNMVEQMHEDIISLWDESLKPCVKLTPLCVTLNCTNVNNSSATNNSMVDDREGLKNCSFNITTELRDKKKQEHALFYRLDIVPINGNSNSNSSVGDYRLINCNVSTIKQACPKMSFDPIPIHYCAPAGFAILKCRDKKFNGTGSCKNVSTVQCTHGIKPVISTQLLLNGSVAEEEIMIRSENFTNNAKNIIVQFNKTIDIMCTRPNNNTRKSISLGPGQAWYATGDIIGNIRQAHCNISGADWGNMIRNVSEKLKEIFNKTTITFKASAGGDLEITTHSFNCRGEFFYCDTSDLFNSSRFNNSSNDTNDTITLPCKIKQIVRMWQRVGQAMYAPPIAGNITCRSNITGLLLTRDGGGNNTNETETFRPAGGDMRDNWRSELYKYKVVKINPLGVAPTRCRRRVVGRRRRRRAIGMGAVLLGFLGTAGSTMGAASMTLTVQVRQLLSGIVQQQSNCLRAPECQQCLLQLTVWGIKQLQARILAVERYLKDQQLLGIWGCSGKLICCTNVPWNASWSNRSYNDIWENLTWVEWEREISNYTQQIYSLLEESQNQQEKNEQDLLALD

SEQ ID NO: 219 - protein, 666 aa, synthetic construct
MDAMKRGLCCVLLLCGAVFVSPSQEIHARFRRGARSNNLWVTVYYGVPVWEDAETPLFCASDAKAYSTEKHNVWATHACVPTDPSPQEISLDNVTENFNMWKNNMVEQMHTDIISLWDESLKPCVKLTPLCVTLNCTNVNNSSATNNSMVDDREGLKNCSFNITTELRDKKKQVHALFYRLDIVPINGNSNSNSSVGDYRLINCNVSTIKQACPKMSFDPIPIHYCAPAGFAILKCRDKKFNGTGSCKNVSTVQCTHGIKPVISTQLLLNGSVAEEEIMIRSENFTNNAKNIIVQFNKTIDIMCTRPNNNTRKSIRLGPGQAIYATGDIIGNIRQAHCNISGADWGNMIRNVSEKLKEIFNKTTITFKASAGGDLEITTHSFNCRGEFFYCDTSDLFNSSRFNNSSNDTNDTITLPCKIKQIVRMWQRVGQAMYAPPIAGNITCRSNITGLLLTRDGGGNNTNETETFRPAGGDMRDNWRSELYKYKVVKINPLGVAPTRCRRRVVGRRRRRRAIGMGAVLLGFLGTAGSTMGAASITLTVQVRNLLSGIVQQQSNLLRAPEAQQHLLQLTVWGIKQLQARILAVERYLRDQQLLGIWGCSGKLICCTNVPWNASWSNRSYNDIWENLTWVEWEREISNYTQQIYSLLEESQNQQEKNEQDLLALD

SEQ ID NO: 220 - protein, 644 aa, synthetic construct
MGSLQPLATLYLLGMLVASVLAAENLWVTVYYGVPVWKEAKTTLFCASDAKAYDPEVHNVWATHACVPTDPNPQEMFLENVTENFNMWKNDMVEQMHQDIISLWSQSLKPCVKLTPLCVTLNCSDVINSTGTMKNCSFSVTTELRDRKQKKHALFYRLDIVPLDENDNSSKDYRLINCNTSTCTQACPKVTFDPIPIHYCAPAGYAILKCNNKTFNGTGPCNNVSTVQCTHGIKPVVSTQLLLNGSLAEEEVIIRSENLTDNVKTIIVQLKEPVGINCTRPNNNIRQSIRIGPGQTFFATGDIIGDIREAHCNISSTKWRETLQQVGGKLAKYFNNKTIKFTQHSGGDLEITTHSFNCRGEFFYCNTSQLFNSTYNETGSINGTGNSTIKLPCRIKQIINMWQGVGQCMYAPPIAGNIICRSKITGLLLTRDGGINKSEEIFRPGGGNMKDNWRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 221 - protein, 639 aa, synthetic construct
MGSLQPLATLYLLGMLVASVLANLWVTVYYGVPVWKEAKTTLFCASDAKAYDPEVHNVWATHACVPTDPNPQEMFLENVTENFNMWKNDMVEQMHQDIISLWSQSLKPCVKLTPLCVTLNCSDVINSTGTMKNCSFSVTTELRDRKQKKHALFYRLDIVPLDENDNSSKDYRLINCNTSTCTQACPKVTFDPIPIHYCAPAGYAILKCNNKTFNGTGPCNNVSTVQCTHGIKPVVSTQLLLNGSLAEEEVIIRSENLTDNVKTIIVQLKEPVGINCTRPNNNIRQSIRIGPGQTFFATGDIIGDIREAHCNISSTKWRETLQQVGGKLAKYFNNKTIKFTQHSGGDLEITTHSFNCRGEFFYCNTSQLFNSTYNETGSINGTGNSTIKLPCRIKQIINMWQGVGQCMYAPPIAGNIICRSKITGLLLTRDGGINKSEEIFRPGGGNMKDNWRSELYKYKVVEIKPLGVAPTKCKRRVVERRRRRRAVVGLGAVFLGFLGAAGSTMGAASITLTVQARQLLSGIVQQQNNLLRAPEAQQHMLQLTVWGIKQLQARVLAIERYLKDQQLLGIWGCSGKLICCTAVPWNSSWSNKSQEDIWDNMTWMQWDREISNYTNTIYRLLEDSQTQQEKNEKDLLALD

SEQ ID NO: 222 - protein, 639 aa, synthetic construct
MGSLQPLATLYLLGMLVASVLANLWVTVYYGVPVWKEACTTLFCASDAKAYDPKVRNVWATHCCVPTDPNPQEMFLENVTENFNMWKNDMVEQMHQDIISLWSQSLKPCVKLTPLCVTLNCSDVINSTGTMKNCSFSVTTELRDRKQKKHALFYRLDIVPLDENDNSSKDYRLINCNTSTCTQACPKVTFDPIPIHYCAPAGYAILKCNNKTFNGTGPCNNVSTVQCTHGIKPVVSTQLLLNGSLAEEEVIIRSENLTDNVKTIIVQLKEPVGINCTRPNNNIRQSIRIGPGQTWFATGDIIGDIREAHCNISSTKWRETLQQVGGKLAKYFNNKTIKFTQHSGGDLEITTHSFNCRGEFFYCNTSQLFNSTYNETGSINGTGNSTIKLPCRIKQIINMWQGVGQCMYAPPIAGNIICRSKITGLLLTRDGGINKSEEIFRPGGGNMKDNWRSELYKYKVVEIKPLGVAPTKCKRRVVERRRRRRAVVGLGAVFLGFLGAAGSTMGAASMTLTVQARQLLSGIVQQQNNCLRAPECQQCMLQLTVWGIKQLQARVLAIERYLKDQQLLGIWGCSGKLICCTAVPWNSSWSNKSQEDIWDNMTWMQWDREISNYTNTIYRLLEDSQTQQEKNEKDLLALD

SEQ ID NO: 223 - protein, 626 aa, synthetic construct
MGSLQPLATLYLLGMLVASVLANLWVTVYYGVPVWKEAKTTLFCASDAKAYDPEVHNVWATHACVPTDPNPQEMFLENVTENFNMWKNDMVEQMHQDIISLWSQSLKPCVKLTPLCVTLNCSDVINSTGTMKNCSFSVTTELRDRKQKKHALFYRLDIVPLDENDNSSKDYRLINCNTSTCTQACPKVTFDPIPIHYCAPAGYAILKCNNKTFNGTGPCNNVSTVQCTHGIKPVVSTQLLLNGSLAEEEVIIRSENLTDNVKTIIVQLKEPVGINCTRPNNNIRQSIRIGPGQTFFATGDIIGDIREAHCNISSTKWRETLQQVGGKLAKYFNNKTIKFTQHSGGDLEITTHSFNCRGEFFYCNTSQLFNSTYNETGSINGTGNSTIKLPCRIKQIINMWQGVGQCMYAPPIAGNIICRSKITGLLLTRDGGINKSEEIFRPGGGNMKDNWRSELYKYKVVEIKPLGVAPTKCKRRVVERRRRRRAVVGLGAVFLGFLGAAGSTMGAASITLTVQARQLLSGNPDWLPDMTVWGIKQLQARVLAIERYLKDQQLLGIWGCSGKLICCTAVPWNSSWSNKSQEDIWDNMTWMQWDREISNYTNTIYRLLEDSQTQQEKNEKDLLALD

SEQ ID NO: 224 - protein, 639 aa, synthetic construct
MGSLQPLATLYLLGMLVASVLANLWVTVYYGVPVWKDAETTLFCASDAKAYDPEKHNVWATHACVPTDPNPQEMFLENVTENFNMWKNDMVEQMHTDIISLWSQSLKPCVKLTPLCVTLNCSDVINSTGTMKNCSFSVTTELRDRKQKVHALFYRLDIVPLDENDNSSKDYRLINCNTSTCTQACPKVTFDPIPIHYCAPAGYAILKCNNKTFNGTGPCNNVSTVQCTHGIKPVVSTQLLLNGSLAEEEVIIRSENLTDNVKTIIVQLKEPVGINCTRPNNNIRQSIRIGPGQTFFATGDIIGDIREAHCNISSTKWRETLQQVGGKLAKYFNNKTIKFTQHSGGDLEITTHSFNCRGEFFYCNTSQLFNSTYNETGSINGTGNSTIKLPCRIKQIINMWQRVGQCMYAPPIAGNIICRSKITGLLLTRDGGINKSEEIFRPGGGNMKDNWRSELYKYKVVEIKPLGVAPTRCKRRVVERRRRRRAVVGLGAVFLGFLGAAGSTMGAASITLTVQARNLLSGIVQQQSNLLRAPEAQQHMLQLTVWGIKQLQARVLAIERYLRDQQLLGIWGCSGKLICCTAVPWNSSWSNKSQEDIWDNMTWMQWDREISNYTNTIYRLLEDSQTQQEKNEKDLLALD

SEQ ID NO: 225 - protein, 657 aa, synthetic construct
MDAMKRGLCCVLLLCGAVFVSPSQEIHARFRRGARAENLWVTVYYGVPVWKEAKTTLFCASDAKAYDPEVHNVWATHACVPTDPNPQEMFLENVTENFNMWKNDMVEQMHQDIISLWSQSLKPCVKLTPLCVTLNCSDVINSTGTMKNCSFSVTTELRDRKQKKHALFYRLDIVPLDENDNSSKDYRLINCNTSTCTQACPKVTFDPIPIHYCAPAGYAILKCNNKTFNGTGPCNNVSTVQCTHGIKPVVSTQLLLNGSLAEEEVIIRSENLTDNVKTIIVQLKEPVGINCTRPNNNIRQSIRIGPGQTFFATGDIIGDIREAHCNISSTKWRETLQQVGGKLAKYFNNKTIKFTQHSGGDLEITTHSFNCRGEFFYCNTSQLFNSTYNETGSINGTGNSTIKLPCRIKQIINMWQGVGQCMYAPPIAGNIICRSKITGLLLTRDGGINKSEEIFRPGGGNMKDNWRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 226 - protein, 652 aa, synthetic construct
MDAMKRGLCCVLLLCGAVFVSPSQEIHARFRRGARNLWVTVYYGVPVWKEAKTTLFCASDAKAYDPEVHNVWATHACVPTDPNPQEMFLENVTENFNMWKNDMVEQMHQDIISLWSQSLKPCVKLTPLCVTLNCSDVINSTGTMKNCSFSVTTELRDRKQKKHALFYRLDIVPLDENDNSSKDYRLINCNTSTCTQACPKVTFDPIPIHYCAPAGYAILKCNNKTFNGTGPCNNVSTVQCTHGIKPVVSTQLLLNGSLAEEEVIIRSENLTDNVKTIIVQLKEPVGINCTRPNNNIRQSIRIGPGQTFFATGDIIGDIREAHCNISSTKWRETLQQVGGKLAKYFNNKTIKFTQHSGGDLEITTHSFNCRGEFFYCNTSQLFNSTYNETGSINGTGNSTIKLPCRIKQIINMWQGVGQCMYAPPIAGNIICRSKITGLLLTRDGGINKSEEIFRPGGGNMKDNWRSELYKYKVVEIKPLGVAPTKCKRRVVERRRRRRAVVGLGAVFLGFLGAAGSTMGAASITLTVQARQLLSGIVQQQNNLLRAPEAQQHMLQLTVWGIKQLQARVLAIERYLKDQQLLGIWGCSGKLICCTAVPWNSSWSNKSQEDIWDNMTWMQWDREISNYTNTIYRLLEDSQTQQEKNEKDLLALD

SEQ ID NO: 227 - protein, 652 aa, synthetic construct
MDAMKRGLCCVLLLCGAVFVSPSQEIHARFRRGARNLWVTVYYGVPVWKEACTTLFCASDAKAYDPKVRNVWATHCCVPTDPNPQEMFLENVTENFNMWKNDMVEQMHQDIISLWSQSLKPCVKLTPLCVTLNCSDVINSTGTMKNCSFSVTTELRDRKQKKHALFYRLDIVPLDENDNSSKDYRLINCNTSTCTQACPKVTFDPIPIHYCAPAGYAILKCNNKTFNGTGPCNNVSTVQCTHGIKPVVSTQLLLNGSLAEEEVIIRSENLTDNVKTIIVQLKEPVGINCTRPNNNIRQSIRIGPGQTWFATGDIIGDIREAHCNISSTKWRETLQQVGGKLAKYFNNKTIKFTQHSGGDLEITTHSFNCRGEFFYCNTSQLFNSTYNETGSINGTGNSTIKLPCRIKQIINMWQGVGQCMYAPPIAGNIICRSKITGLLLTRDGGINKSEEIFRPGGGNMKDNWRSELYKYKVVEIKPLGVAPTKCKRRVVERRRRRRAVVGLGAVFLGFLGAAGSTMGAASMTLTVQARQLLSGIVQQQNNCLRAPECQQCMLQLTVWGIKQLQARVLAIERYLKDQQLLGIWGCSGKLICCTAVPWNSSWSNKSQEDIWDNMTWMQWDREISNYTNTIYRLLEDSQTQQEKNEKDLLALD

SEQ ID NO: 228 - protein, 639 aa, synthetic construct
MDAMKRGLCCVLLLCGAVFVSPSQEIHARFRRGARNLWVTVYYGVPVWKEAKTTLFCASDAKAYDPEVHNVWATHACVPTDPNPQEMFLENVTENFNMWKNDMVEQMHQDIISLWSQSLKPCVKLTPLCVTLNCSDVINSTGTMKNCSFSVTTELRDRKQKKHALFYRLDIVPLDENDNSSKDYRLINCNTSTCTQACPKVTFDPIPIHYCAPAGYAILKCNNKTFNGTGPCNNVSTVQCTHGIKPVVSTQLLLNGSLAEEEVIIRSENLTDNVKTIIVQLKEPVGINCTRPNNNIRQSIRIGPGQTFFATGDIIGDIREAHCNISSTKWRETLQQVGGKLAKYFNNKTIKFTQHSGGDLEITTHSFNCRGEFFYCNTSQLFNSTYNETGSINGTGNSTIKLPCRIKQIINMWQGVGQCMYAPPIAGNIICRSKITGLLLTRDGGINKSEEIFRPGGGNMKDNWRSELYKYKVVEIKPLGVAPTKCKRRVVERRRRRRAVVGLGAVFLGFLGAAGSTMGAASITLTVQARQLLSGNPDWLPDMTVWGIKQLQARVLAIERYLKDQQLLGIWGCSGKLICCTAVPWNSSWSNKSQEDIWDNMTWMQWDREISNYTNTIYRLLEDSQTQQEKNEKDLLALD

SEQ ID NO: 229 - protein, 652 aa, synthetic construct
MDAMKRGLCCVLLLCGAVFVSPSQEIHARFRRGARNLWVTVYYGVPVWKDAETTLFCASDAKAYDPEKHNVWATHACVPTDPNPQEMFLENVTENFNMWKNDMVEQMHTDIISLWSQSLKPCVKLTPLCVTLNCSDVINSTGTMKNCSFSVTTELRDRKQKVHALFYRLDIVPLDENDNSSKDYRLINCNTSTCTQACPKVTFDPIPIHYCAPAGYAILKCNNKTFNGTGPCNNVSTVQCTHGIKPVVSTQLLLNGSLAEEEVIIRSENLTDNVKTIIVQLKEPVGINCTRPNNNIRQSIRIGPGQTFFATGDIIGDIREAHCNISSTKWRETLQQVGGKLAKYFNNKTIKFTQHSGGDLEITTHSFNCRGEFFYCNTSQLFNSTYNETGSINGTGNSTIKLPCRIKQIINMWQRVGQCMYAPPIAGNIICRSKITGLLLTRDGGINKSEEIFRPGGGNMKDNWRSELYKYKVVEIKPLGVAPTRCKRRVVERRRRRRAVVGLGAVFLGFLGAAGSTMGAASITLTVQARNLLSGIVQQQSNLLRAPEAQQHMLQLTVWGIKQLQARVLAIERYLRDQQLLGIWGCSGKLICCTAVPWNSSWSNKSQEDIWDNMTWMQWDREISNYTNTIYRLLEDSQTQQEKNEKDLLALD

SEQ ID NO: 230 - protein, 649 aa, synthetic construct
MPMGSLQPLATLYLLGMLVASVLAKGKLWVTVYYGVPVWKEAKTTLFCASDARAYEKEVHNVWATHACVPTDPSPQELFLKNVTENFNMWKNDMVDQMHEDIISLWDQSLKPCVKLTPLCVTLICSNATVKNRTVEEMKNCSFNTTTEIRDKEKKEYALFYKPDVVPLDETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTPVEIVCTRPNNNTRKSVRIGPGQTFYATGDIIGDIRQAHCNISEKQWNDTLQKVGKELKKHFPNKTIKYAQSAGGDMEITTHSFNCGGEFFYCNTAKLFNGTYNGTDISTNSSADSNSTITLQCRIKQIINMWQGVGRCMYAPPIAGNITCKSHITGLLLTRDGGTNSSGKEEIFRPAGGDMRDNWRSELYKYKVVEIQPLGIAPTGCKRRVVERRRRRRAAGLGALFLGFLGAAGSTMGAASITLTVQARQLLSGIVQQQSNLLRAPEAQQHMLQLTVWGIKQLQARVLALERYLKDQQLLGMWGCSGKLICCTNVPWNTSWSNKSETDIWDNMTWMQWEREISNYTETIYKLLEDSQNQQERNEQDLLALD

SEQ ID NO: 231 - protein, 660 aa, synthetic construct
MDAMKRGLCCVLLLCGAVFVSPSQEIHARFRRGARKGKLWVTVYYGVPVWKEAKTTLFCASDARAYEKEVHNVWATHACVPTDPSPQELFLKNVTENFNMWKNDMVDQMHEDIISLWDQSLKPCVKLTPLCVTLICSNATVKNRTVEEMKNCSFNTTTEIRDKEKKEYALFYKPDVVPLDETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTPVEIVCTRPNNNTRKSVRIGPGQTFYATGDIIGDIRQAHCNISEKQWNDTLQKVGKELKKHFPNKTIKYAQSAGGDMEITTHSFNCGGEFFYCNTAKLFNGTYNGTDISTNSSADSNSTITLQCRIKQIINMWQGVGRCMYAPPIAGNITCKSHITGLLLTRDGGTNSSGKEEIFRPAGGDMRDNWRSELYKYKVVEIQPLGIAPTGCKRRVVERRRRRRAAGLGALFLGFLGAAGSTMGAASITLTVQARQLLSGIVQQQSNLLRAPEAQQHMLQLTVWGIKQLQARVLALERYLKDQQLLGMWGCSGKLICCTNVPWNTSWSNKSETDIWDNMTWMQWEREISNYTETIYKLLEDSQNQQERNEQDLLALD

SEQ ID NO: 232 - protein, 649 aa, synthetic construct
MPMGSLQPLATLYLLGMLVASVLAAENLWVTVYYGVPVWKEAKTTLFCASDARAYEKEVHNVWATHACVPTDPSPQELFLKNVTENFNMWKNDMVDQMHEDIISLWDQSLKPCVKLTPLCVTLICSNATVKNRTVEEMKNCSFNTTTEIRDKEKKEYALFYKPDVVPLDETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTPVEIVCTRPNNNTRKSVRIGPGQTFYATGDIIGDIRQAHCNISEKQWNDTLQKVGKELKKHFPNKTIKYAQSAGGDMEITTHSFNCGGEFFYCNTAKLFNGTYNGTDISTNSSADSNSTITLQCRIKQIINMWQGVGRCMYAPPIAGNITCKSHITGLLLTRDGGTNSSGKEEIFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 233 - protein, 663 aa, synthetic construct
MPMGSLQPLATLYLLGMLVASVLAEGNLWVTVYYGVPVWKEAKTTLFCASDAKAYKKEVHNVWATHACVPTDPSPQELFLKNVTENFNMWKNDMVDQMHEDIISLWDQSLKPCVKLTPLCVTLICSNAIVKNSTTEKMSNVTVNNITIEEMKNCSFNTTTEIRDKIKKERALFYRPDIVPLNDETNNTSKYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHAPVEIVCTRPYNNTRKSVRIGPGQTFYATGDIIGDPRQAHCNISKEIWNKTLQEVGKELQKHFPNKTIRYNQSAGGDMEITTHSFNCGGEFFYCNTSNLFNSTYNDTYISPNSTNSTSIITLQCKIKQIINMWQGVGRCMYAPPIAGNITCKSNITGLLLTRDGGINNVSNETETFRPAGGDMRDNWRSELYKYKVVEVQPLGIAPTGCKRRVVERRRRRRAVGLGALFLGFLGAAGSTMGAASITLTVQARQLLSGIVQQQSNLLRAPEAQQHMLQLTVWGIKQLQARVLALERYLKDQQLLGIWGCSGKLICCTNVPWNTSWSNKSETDIWNNMTWMQWEREISNYTGTIYKLLEDSQNQQERNEKDLLALD

SEQ ID NO: 234 - protein, 674 aa, synthetic construct
MDAMKRGLCCVLLLCGAVFVSPSQEIHARFRRGAREGNLWVTVYYGVPVWKEAKTTLFCASDAKAYKKEVHNVWATHACVPTDPSPQELFLKNVTENFNMWKNDMVDQMHEDIISLWDQSLKPCVKLTPLCVTLICSNAIVKNSTTEKMSNVTVNNITIEEMKNCSFNTTTEIRDKIKKERALFYRPDIVPLNDETNNTSKYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHAPVEIVCTRPYNNTRKSVRIGPGQTFYATGDIIGDPRQAHCNISKEIWNKTLQEVGKELQKHFPNKTIRYNQSAGGDMEITTHSFNCGGEFFYCNTSNLFNSTYNDTYISPNSTNSTSIITLQCKIKQIINMWQGVGRCMYAPPIAGNITCKSNITGLLLTRDGGINNVSNETETFRPAGGDMRDNWRSELYKYKVVEVQPLGIAPTGCKRRVVERRRRRRAVGLGALFLGFLGAAGSTMGAASITLTVQARQLLSGIVQQQSNLLRAPEAQQHMLQLTVWGIKQLQARVLALERYLKDQQLLGIWGCSGKLICCTNVPWNTSWSNKSETDIWNNMTWMQWEREISNYTGTIYKLLEDSQNQQERNEKDLLALD

SEQ ID NO: 235 - protein, 663 aa, synthetic construct
MPMGSLQPLATLYLLGMLVASVLAAENLWVTVYYGVPVWKEAKTTLFCASDAKAYKKEVHNVWATHACVPTDPSPQELFLKNVTENFNMWKNDMVDQMHEDIISLWDQSLKPCVKLTPLCVTLICSNAIVKNSTTEKMSNVTVNNITIEEMKNCSFNTTTEIRDKIKKERALFYRPDIVPLNDETNNTSKYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHAPVEIVCTRPYNNTRKSVRIGPGQTFYATGDIIGDPRQAHCNISKEIWNKTLQEVGKELQKHFPNKTIRYNQSAGGDMEITTHSFNCGGEFFYCNTSNLFNSTYNDTYISPNSTNSTSIITLQCKIKQIINMWQGVGRCMYAPPIAGNITCKSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 236 - protein, 650 aa, synthetic construct
MPMGSLQPLATLYLLGMLVASVLAEGNLWVTVYYGVPVWKEAKTTLFCASDAKAYVKEVHNVWATHACVPTDPSPQELFLKNVTENFNMWKNDMVDQMHEDIISLWDQSLKPCVKLTPLCVTLICSTATVNNSKFEEMKNCSFNTTTEIRDKEKKEYALFYRPDIVPLDNETSNISEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGNPRQAHCNISKERWNDTLQKVGKELQKHFPNKTIKYNQSAGGDMEITTHSFNCGGEFFYCNTSNLFNSTYNDTYISTNSTNSTSYITLQCRIKQIINMWQGVGRCMYAPPIAGNITCKSNITGLLLTRDGGINKDSNETETFRPAGGDMRDNWRSELYKYKVVEVQPLGIAPTGCKRRVVERRRRRRAAGLGALFLGFLGAAGSTMGAASITLTVQARQLLSGIVQQQSNLLRAPEAQQHMLQLTVWGIKQLQARVLALERYLKDQQLLGMWGCSGKLICCTNVPWNTSWSNKSETDIWDNMTWMQWEREISNYTGTIYKLLEDSQNQQERNEQDLLALD

SEQ ID NO: 237 - protein, 661 aa, synthetic construct
MDAMKRGLCCVLLLCGAVFVSPSQEIHARFRRGAREGNLWVTVYYGVPVWKEAKTTLFCASDAKAYVKEVHNVWATHACVPTDPSPQELFLKNVTENFNMWKNDMVDQMHEDIISLWDQSLKPCVKLTPLCVTLICSTATVNNSKFEEMKNCSFNTTTEIRDKEKKEYALFYRPDIVPLDNETSNISEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGNPRQAHCNISKERWNDTLQKVGKELQKHFPNKTIKYNQSAGGDMEITTHSFNCGGEFFYCNTSNLFNSTYNDTYISTNSTNSTSYITLQCRIKQIINMWQGVGRCMYAPPIAGNITCKSNITGLLLTRDGGINKDSNETETFRPAGGDMRDNWRSELYKYKVVEVQPLGIAPTGCKRRVVERRRRRRAAGLGALFLGFLGAAGSTMGAASITLTVQARQLLSGIVQQQSNLLRAPEAQQHMLQLTVWGIKQLQARVLALERYLKDQQLLGMWGCSGKLICCTNVPWNTSWSNKSETDIWDNMTWMQWEREISNYTGTIYKLLEDSQNQQERNEQDLLALD

SEQ ID NO: 238 - protein, 650 aa, synthetic construct
MPMGSLQPLATLYLLGMLVASVLAAENLVTVYYGVPVWKEAKTTLFCASDAKAYVKEVHNVWATHACVPTDPSPQELFLKNVTENFNMWKNDMVDQMHEDIISLWDQSLKPCVKLTPLCVTLICSTATVNNSKFEEMKNCSFNTTTEIRDKEKKEYALFYRPDIVPLDNETSNISEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGNPRQAHCNISKERWNDTLQKVGKELQKHFPNKTIKYNQSAGGDMEITTHSFNCGGEFFYCNTSNLFNSTYNDTYISTNSTNSTSYITLQCRIKQIINMWQGVGRCMYAPPIAGNITCKSNITGLLLTRDGGINKDSNETETFRPAGGDMRDNWRRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 239 - DNA, 1999 bp, synthetic construct
  source 1..1999: mol_type = other DNA
gtcgacaagcttcccgggccaccatgcccatgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgccaaggcctacgagaaggaggtgcacaacgtgtgggccacccacgcctgcgtgcccaccgacccctccccccaggagctggtgctgaagaacgtgaccgagaacttcaacatgtggaagaacgacatggtggaccagatgcacgaggacatcatctccctgtgggaccagtccctgaagccctgcgtgaagctgacccccctgtgcgtgaccctgaactgctccaacgcccgctccaacgtgaacgtgacctccatcaacaacaccatcatgggcgagatgaagaactgctccttcaacaccaccaccgagatccgcgacaaggagaagaaggagtacgccctgttctacaagcccgacgtggtgcccctgaacgagacctccaacacctccgagtaccgcctgatcaactgcaacacctccgcctgcacccaggcctgccccaaggtgaccttcgagcccatccccatccactactgcgcccccgccggctacgccatcctgaagtgcaacaacaagaccttcaacggcaccggcccctgctccaacgtgtccaccgtgcagtgcacccacggcatccgccccgtggtgtccacccagctgctgctgaacggctccctggccgagaaggagatcgtgatccgctccgagaacctgaccaacaacgccaagatcatcatcgtgcacctgaacacctccgtggagatcgtgtgcacccgccccggcaacaacacccgcaagtccgtgcgcatcggccccggccagaccttctacgccaccggcgacatcatcggcgacatccgccaggcccactgcaacatctccgagggccagtggaacaagaccctgcacgaggtgtccaaggagctgcagaagcacttccccaacaagaccatcaagtacgagcgctccgccggcggcgacatggagatcgccacccactccttcaactgcggcggcgagttcttctactgcaacacctccaacctgttcaacggcacctacaacggcacctacatcaacacctcctccacctcctacatcaccctgcagtgccgcatcaagcagatcatcaacatgtggcagggcgtgggccgctgcatgtacgccccccccatcgccggcaacatcacctgcaagtccaacatcaccggcctgctgctgacccgcgacggcggcaccaagaacaactccaacgaggagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggactagtaagcggccgcggatcc

SEQ ID NO: 240 - DNA, 1999 bp, synthetic construct
  source 1..1999: mol_type = other DNA
gtcgacaagcttcccgggccaccatgcccatgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgccaaggcctacgagaagaaggtgcacaacgtgtgggccacccacgcctgcgtgcccaccgacccctccccccaggagctggtgctgaagaacgtgaccgagaacttcaacatgtggaagaacgacatggtggaccagatgcacgaggacatcatctccctgtgggaccagtccctgaagccctgcgtgaagctgacccccctgtgcgtgaccctgaactgctccaacgcccgctccaacgtgaacgtgacctccatcaacaacaccatcatgggcgagatgaagaactgctccttcaacaccaccaccgagatccgcgacaaggagaagaaggagtacgccctgttctacaagcccgacgtggtgcccctgaacgagacctccaacacctccgagtaccgcctgatcaactgcaacacctccgccgtgacccaggcctgccccaaggtgaccttcgagcccatccccatccactactgcgcccccgccggctacgccatcctgaagtgcaacaacaagaccttcaacggcaccggcccctgctccaacgtgtccaccgtgcagtgcacccacggcatccgccccgtggtgtccacccagctgctgctgaacggctccctggccgagaaggagatcgtgatccgctccgagaacctgaccaacaacgccaagatcatcatcgtgcacctgaacacctccgtggagatcgtgtgcacccgccccggcaacaacacccgcaagtccgtgcgcatcggccccggccagtggttctacgccaccggcgacatcatcggcgacatccgccaggcccactgcaacatctccgagggccagtggaacaagaccctgcacgaggtgtccaaggagctgcagaagcacttccccaacaagaccatcaagtacgagcgctccgccggcggcgacatggagatcgccacccactccttcaactgcggcggcgagttcttctactgcaacacctccaacctgttcaacggcacctacaacggcacctacatcaacacctcctccacctcctacatcaccctgcagtgccgcatcaagcagatcatcaacatgtggcagggcgtgggccgcgccatgtacgccccccccatcgccggcaacatcacctgcaagtccaacatcaccggcctgctgctgacccgcgacggcggcaccaagaacaactccaacgaggagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggactagtaagcggccgcggatcc

SEQ ID NO: 241 - DNA, 1999 bp, synthetic construct
  source 1..1999: mol_type = other DNA
gtcgacaagcttcccgggccaccatgcccatgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgccaaggcctacgagaaggaggtgcacaacgtgtgggccacccacgcctgcgtgcccaccgacccctccccccaggagctggtgctgaagaacgtgaccgagaacttcaacatgtggaagaacgacatggtggaccagatgcacgaggacatcatctccctgtgggaccagtccctgaagccctgcgtgaagctgacccccctgtgcgtgaccctgaactgctccaacgcccgctccaacgtgaacgtgacctccatcaacaacaccatcatgggcgagatgaagaactgctccttcaacaccaccaccgagatccgcgacaaggagaagaaggagtacgccctgttctacaagcccgacgtggtgcccctgaacgagacctccaacacctccgagtaccgcctgatcaactgcaacacctccgcctgcacccaggcctgccccaaggtgaccttcgagcccatccccatccactactgcgcccccgccggctacgccatcctgaagtgcaacaacaagaccttcaacggcaccggcccctgctccaacgtgtccaccgtgcagtgcacccacggcatccgccccgtggtgtccacccagctgctgctgaacggctccctggccgagaaggagatcgtgatccgctccgagaacctgaccaacaacgccaagatcatcatcgtgcacctgaacacctccgtggagatcgtgtgcacccgccccggcaacaacacccgcaagtccgtgcgcatcggccccggccagaccttctacgccaccggcgacatcatcggcgacatccgccaggcccactgcaacatctccgagggccagtggaacaagaccctgcacgaggtgtccaaggagctgcagaagcacttccccaacaagaccatcaagtacgagcgctccgccggcggccgcatggagatcgccacccactccttcaactgcggcggcgagttcttctactgcaacacctccaacctgttcaacggcacctacaacggcacctacatcaacacctcctccacctcctacatcaccctgcagtgccgcatcaagcagatcatcaacatgtggcagggcgtgggccgctgcatgtacgccccccccatcgccggcaacatcacctgcaagtccaacatcaccggcctgctgctgacccgcgacggcggcaccaagaacaactccaacgaggagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggactagtaagcggccgcggatcc

SEQ ID NO: 242 - DNA, 2017 bp, synthetic construct
  source 1..2017: mol_type = other DNA
gtcgacaagcttcccgggccaccatgcccatgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgccaaggcctacgagaaggaggtgcacaacgtgtgggccacccacgcctgcgtgcccaccgacccctccccccaggagctggtgctgaagaacgtgaccgagaacttcaacatgtggaagaacgacatggtggaccagatgcacgaggacatcatctccctgtgggaccagtccctgaagccctgcgtgaagctgacccccctgtgcgtgaccctgaactgctccaacgcccgctccaacgtgaacgtgacctccatcaacaacaccatcatgggcgagatgaagaactgctccttcaacaccaccaccgagatccgcgacaaggagaagaaggagtacgccctgttctacaagcccgacgtggtgcccctgaacgagacctccaacacctccgagtaccgcctgatcaactgcaacacctccgcctgcacccaggcctgccccaaggtgaccttcgagcccatccccatccactactgcgcccccgccggctacgccatcctgaagtgcaacaacaagaccttcaacggcaccggcccctgctccaacgtgtccaccgtgcagtgcacccacggcatccgccccgtggtgtccacccagctgctgctgaacggctccctggccgagaaggagatcgtgatccgctccgagaacctgaccaacaacgccaagatcatcatcgtgcacctgaacacctccgtggagatcgtgtgcacccgccccggcaacaacacccgcaagtccgtgcgcatcggccccggccagaccttctacgccaccggcgacatcatcggcgacatccgccaggcccactgcaacatctccgagggccagtggaacaagaccctgcacgaggtgtccaaggagctgcagaagcacttccccaacaagaccatcaagtacgagcgctccgccggcggcgacatggagatcgccacccactccttcaactgcggcggcgagttcttctactgcaacacctccaacctgttcaacggcacctacaacggcacctacatcaacacctcctccacctcctacatcaccctgcagtgccgcatcaagcagatcatcaacatgtggcagggcgtgggccgctgcatgtacgccccccccatcgccggcaacatcacctgcaagtccaacatcaccggcctgctgctgacccgcgacggcggcaccaagaacaactccaacgaggagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacctgcctgagaccggaggatagtaagcggccgcggatcc

SEQ ID NO: 243 - DNA, 2503 bp, synthetic construct
  source 1..2503: mol_type = other DNA
gtcgacaagcttcccgggccaccatgcccatgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgccaaggcctacgagaaggaggtgcacaacgtgtgggccacccacgcctgcgtgcccaccgacccctccccccaggagctggtgctgaagaacgtgaccgagaacttcaacatgtggaagaacgacatggtggaccagatgcacgaggacatcatctccctgtgggaccagtccctgaagccctgcgtgaagctgacccccctgtgcgtgaccctgaactgctccaacgcccgctccaacgtgaacgtgacctccatcaacaacaccatcatgggcgagatgaagaactgctccttcaacaccaccaccgagatccgcgacaaggagaagaaggagtacgccctgttctacaagcccgacgtggtgcccctgaacgagacctccaacacctccgagtaccgcctgatcaactgcaacacctccgcctgcacccaggcctgccccaaggtgaccttcgagcccatccccatccactactgcgcccccgccggctacgccatcctgaagtgcaacaacaagaccttcaacggcaccggcccctgctccaacgtgtccaccgtgcagtgcacccacggcatccgccccgtggtgtccacccagctgctgctgaacggctccctggccgagaaggagatcgtgatccgctccgagaacctgaccaacaacgccaagatcatcatcgtgcacctgaacacctccgtggagatcgtgtgcacccgccccggcaacaacacccgcaagtccgtgcgcatcggccccggccagaccttctacgccaccggcgacatcatcggcgacatccgccaggcccactgcaacatctccgagggccagtggaacaagaccctgcacgaggtgtccaaggagctgcagaagcacttccccaacaagaccatcaagtacgagcgctccgccggcggcgacatggagatcgccacccactccttcaactgcggcggcgagttcttctactgcaacacctccaacctgttcaacggcacctacaacggcacctacatcaacacctcctccacctcctacatcaccctgcagtgccgcatcaagcagatcatcaacatgtggcagggcgtgggccgctgcatgtacgccccccccatcgccggcaacatcacctgcaagtccaacatcaccggcctgctgctgacccgcgacggcggcaccaagaacaactccaacgaggagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacggtggtggaagcggagatatcatcaagctgctgaacgagcaagtgaacaaagagatgaacagctccaacctgtacatgagcatgtccagctggtgctacacccacagccttgatggcgccggactgttcctgtttgatcacgccgccgaggaatacgagcacgccaagaagctgatcattttcctgaacgagaacaacgtgccagtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctagtaagcggccgcggatcc

SEQ ID NO: 244 - DNA, 2029 bp, synthetic construct
  source 1..2029: mol_type = other DNA
gtcgacaagcttcccgggccaccatgcccatgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgccaaggcctacaagaaggaggtgcacaacgtgtgggccacccacgcctgcgtgcccaccgacccctccccccaggagctgttcctgaagaacgtgaccgagaacttcaacatgtggaagaacgacatggtggaccagatgcacgaggacatcatctccctgtgggaccagtccctgaagccctgcgtgaagctgacccccctgtgcgtgaccctgatctgctccaacgccatcgtgaagaactccaccaccgaggagatgtccaccgccctggtgaagaactccaccaccgaggccatgaagaactgctccttcaacaccaccaccgagatccgcgacaaggagaagaaggagtacgccctgttctaccgccccgacatcgtgcccctgaacaacgagaccggcaacatctccgagtaccgcctgatcaactgcaacacctccgcctgcacccaggcctgccccaaggtgaccttcgagcccatccccatccactactgcgcccccgccggctacgccatcctgaagtgcaacgacgagaccttcaacggcaccggcccctgctccaacgtgtccaccgtgcagtgcacccacggcatccgccccgtggtgtccacccagctgctgctgaacggctccctggccaaggaggagatcgtgatccgctccgagaacctgaccaacaacgccaagatcatcatcgtgcacctgcacacccccgtggagatcgtgtgcacccgccccaacaacaacacccgcaagtccgtgcgcatcggccccggccagaccttctacgccaccggcgacatcatcggcgacccccgcaaggcccactgcaacatctccgagaaggactggaacaagaccctgcaggaggtgggcaaggagctgcagaagcacttccccaacaagaccatccgctacaaccagtccgccggcggcgacatggagatcaccacccactccttcaactgcggcggcgagttcttctactgcaacacctccaagctgttcaactccacctacaacgacacctacatctccaccaactcctccgccaacaactcctccaccatcaccctgcagtgccgcatcaagcagatcatcaacatgtggcagggcgtgggccgctgcatgtacgccccccccatcgccggcaacatcacctgcaagtccaacatcaccggcctgctgctgacccgcgacggcggccccgactccaacgagaccgagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggactagtaagcggccgcggatcc

SEQ ID NO: 245 - DNA, 2029 bp, synthetic construct
  source 1..2029: mol_type = other DNA
gtcgacaagcttcccgggccaccatgcccatgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgccaaggcctacaagaagaaggtgcacaacgtgtgggccacccacgcctgcgtgcccaccgacccctccccccaggagctgttcctgaagaacgtgaccgagaacttcaacatgtggaagaacgacatggtggaccagatgcacgaggacatcatctccctgtgggaccagtccctgaagccctgcgtgaagctgacccccctgtgcgtgaccctgatctgctccaacgccatcgtgaagaactccaccaccgaggagatgtccaccgccctggtgaagaactccaccaccgaggccatgaagaactgctccttcaacaccaccaccgagatccgcgacaaggagaagaaggagtacgccctgttctaccgccccgacatcgtgcccctgaacaacgagaccggcaacatctccgagtaccgcctgatcaactgcaacacctccgccgtgacccaggcctgccccaaggtgaccttcgagcccatccccatccactactgcgcccccgccggctacgccatcctgaagtgcaacgacgagaccttcaacggcaccggcccctgctccaacgtgtccaccgtgcagtgcacccacggcatccgccccgtggtgtccacccagctgctgctgaacggctccctggccaaggaggagatcgtgatccgctccgagaacctgaccaacaacgccaagatcatcatcgtgcacctgcacacccccgtggagatcgtgtgcacccgccccaacaacaacacccgcaagtccgtgcgcatcggccccggccagtggttctacgccaccggcgacatcatcggcgacccccgcaaggcccactgcaacatctccgagaaggactggaacaagaccctgcaggaggtgggcaaggagctgcagaagcacttccccaacaagaccatccgctacaaccagtccgccggcggcgacatggagatcaccacccactccttcaactgcggcggcgagttcttctactgcaacacctccaagctgttcaactccacctacaacgacacctacatctccaccaactcctccgccaacaactcctccaccatcaccctgcagtgccgcatcaagcagatcatcaacatgtggcagggcgtgggccgcgccatgtacgccccccccatcgccggcaacatcacctgcaagtccaacatcaccggcctgctgctgacccgcgacggcggccccgactccaacgagaccgagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggactagtaagcggccgcggatcc

SEQ ID NO: 246 - DNA, 2029 bp, synthetic construct
  source 1..2029: mol_type = other DNA
gtcgacaagcttcccgggccaccatgcccatgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgccaaggcctacaagaaggaggtgcacaacgtgtgggccacccacgcctgcgtgcccaccgacccctccccccaggagctgttcctgaagaacgtgaccgagaacttcaacatgtggaagaacgacatggtggaccagatgcacgaggacatcatctccctgtgggaccagtccctgaagccctgcgtgaagctgacccccctgtgcgtgaccctgatctgctccaacgccatcgtgaagaactccaccaccgaggagatgtccaccgccctggtgaagaactccaccaccgaggccatgaagaactgctccttcaacaccaccaccgagatccgcgacaaggagaagaaggagtacgccctgttctaccgccccgacatcgtgcccctgaacaacgagaccggcaacatctccgagtaccgcctgatcaactgcaacacctccgcctgcacccaggcctgccccaaggtgaccttcgagcccatccccatccactactgcgcccccgccggctacgccatcctgaagtgcaacgacgagaccttcaacggcaccggcccctgctccaacgtgtccaccgtgcagtgcacccacggcatccgccccgtggtgtccacccagctgctgctgaacggctccctggccaaggaggagatcgtgatccgctccgagaacctgaccaacaacgccaagatcatcatcgtgcacctgcacacccccgtggagatcgtgtgcacccgccccaacaacaacacccgcaagtccgtgcgcatcggccccggccagaccttctacgccaccggcgacatcatcggcgacccccgcaaggcccactgcaacatctccgagaaggactggaacaagaccctgcaggaggtgggcaaggagctgcagaagcacttccccaacaagaccatccgctacaaccagtccgccggcggccgcatggagatcaccacccactccttcaactgcggcggcgagttcttctactgcaacacctccaagctgttcaactccacctacaacgacacctacatctccaccaactcctccgccaacaactcctccaccatcaccctgcagtgccgcatcaagcagatcatcaacatgtggcagggcgtgggccgctgcatgtacgccccccccatcgccggcaacatcacctgcaagtccaacatcaccggcctgctgctgacccgcgacggcggccccgactccaacgagaccgagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggactagtaagcggccgcggatcc

SEQ ID NO: 247 - DNA, 2047 bp, synthetic construct
  source 1..2047: mol_type = other DNA
gtcgacaagcttcccgggccaccatgcccatgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgccaaggcctacaagaaggaggtgcacaacgtgtgggccacccacgcctgcgtgcccaccgacccctccccccaggagctgttcctgaagaacgtgaccgagaacttcaacatgtggaagaacgacatggtggaccagatgcacgaggacatcatctccctgtgggaccagtccctgaagccctgcgtgaagctgacccccctgtgcgtgaccctgatctgctccaacgccatcgtgaagaactccaccaccgaggagatgtccaccgccctggtgaagaactccaccaccgaggccatgaagaactgctccttcaacaccaccaccgagatccgcgacaaggagaagaaggagtacgccctgttctaccgccccgacatcgtgcccctgaacaacgagaccggcaacatctccgagtaccgcctgatcaactgcaacacctccgcctgcacccaggcctgccccaaggtgaccttcgagcccatccccatccactactgcgcccccgccggctacgccatcctgaagtgcaacgacgagaccttcaacggcaccggcccctgctccaacgtgtccaccgtgcagtgcacccacggcatccgccccgtggtgtccacccagctgctgctgaacggctccctggccaaggaggagatcgtgatccgctccgagaacctgaccaacaacgccaagatcatcatcgtgcacctgcacacccccgtggagatcgtgtgcacccgccccaacaacaacacccgcaagtccgtgcgcatcggccccggccagaccttctacgccaccggcgacatcatcggcgacccccgcaaggcccactgcaacatctccgagaaggactggaacaagaccctgcaggaggtgggcaaggagctgcagaagcacttccccaacaagaccatccgctacaaccagtccgccggcggcgacatggagatcaccacccactccttcaactgcggcggcgagttcttctactgcaacacctccaagctgttcaactccacctacaacgacacctacatctccaccaactcctccgccaacaactcctccaccatcaccctgcagtgccgcatcaagcagatcatcaacatgtggcagggcgtgggccgctgcatgtacgccccccccatcgccggcaacatcacctgcaagtccaacatcaccggcctgctgctgacccgcgacggcggccccgactccaacgagaccgagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacctgcctgagaccggaggatagtaagcggccgcggatcc

SEQ ID NO: 248 - DNA, 2533 bp, synthetic construct
  source 1..2533: mol_type = other DNA
gtcgacaagcttcccgggccaccatgcccatgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgccaaggcctacaagaaggaggtgcacaacgtgtgggccacccacgcctgcgtgcccaccgacccctccccccaggagctgttcctgaagaacgtgaccgagaacttcaacatgtggaagaacgacatggtggaccagatgcacgaggacatcatctccctgtgggaccagtccctgaagccctgcgtgaagctgacccccctgtgcgtgaccctgatctgctccaacgccatcgtgaagaactccaccaccgaggagatgtccaccgccctggtgaagaactccaccaccgaggccatgaagaactgctccttcaacaccaccaccgagatccgcgacaaggagaagaaggagtacgccctgttctaccgccccgacatcgtgcccctgaacaacgagaccggcaacatctccgagtaccgcctgatcaactgcaacacctccgcctgcacccaggcctgccccaaggtgaccttcgagcccatccccatccactactgcgcccccgccggctacgccatcctgaagtgcaacgacgagaccttcaacggcaccggcccctgctccaacgtgtccaccgtgcagtgcacccacggcatccgccccgtggtgtccacccagctgctgctgaacggctccctggccaaggaggagatcgtgatccgctccgagaacctgaccaacaacgccaagatcatcatcgtgcacctgcacacccccgtggagatcgtgtgcacccgccccaacaacaacacccgcaagtccgtgcgcatcggccccggccagaccttctacgccaccggcgacatcatcggcgacccccgcaaggcccactgcaacatctccgagaaggactggaacaagaccctgcaggaggtgggcaaggagctgcagaagcacttccccaacaagaccatccgctacaaccagtccgccggcggcgacatggagatcaccacccactccttcaactgcggcggcgagttcttctactgcaacacctccaagctgttcaactccacctacaacgacacctacatctccaccaactcctccgccaacaactcctccaccatcaccctgcagtgccgcatcaagcagatcatcaacatgtggcagggcgtgggccgctgcatgtacgccccccccatcgccggcaacatcacctgcaagtccaacatcaccggcctgctgctgacccgcgacggcggccccgactccaacgagaccgagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacggtggtggaagcggagatatcatcaagctgctgaacgagcaagtgaacaaagagatgaacagctccaacctgtacatgagcatgtccagctggtgctacacccacagccttgatggcgccggactgttcctgtttgatcacgccgccgaggaatacgagcacgccaagaagctgatcattttcctgaacgagaacaacgtgccagtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctagtaagcggccgcggatcc

SEQ ID NO: 249 - protein, 652 aa, synthetic construct
MPMGSLQPLATLYLLGMLVASVLAAENLWVTVYYGVPVWKEAKTTLFCASDAKAYEKEVHNVWATHACVPTDPSPQELVLKNVTENFNMWKNDMVDQMHEDIISLWDQSLKPCVKLTPLCVTLNCSNARSNVNVTSINNTIMGEMKNCSFNTTTEIRDKEKKEYALFYKPDVVPLNETSNTSEYRLINCNTSACTQACPKVTFEPIPIHYCAPAGYAILKCNNKTFNGTGPCSNVSTVQCTHGIRPVVSTQLLLNGSLAEKEIVIRSENLTNNAKIIIVHLNTSVEIVCTRPGNNTRKSVRIGPGQTFYATGDIIGDIRQAHCNISEGQWNKTLHEVSKELQKHFPNKTIKYERSAGGDMEIATHSFNCGGEFFYCNTSNLFNGTYNGTYINTSSTSYITLQCRIKQIINMWQGVGRCMYAPPIAGNITCKSNITGLLLTRDGGTKNNSNEETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 250 - protein, 652 aa, synthetic construct
MPMGSLQPLATLYLLGMLVASVLAAENLWVTVYYGVPVWKEAKTTLFCASDAKAYEKKVHNVWATHACVPTDPSPQELVLKNVTENFNMWKNDMVDQMHEDIISLWDQSLKPCVKLTPLCVTLNCSNARSNVNVTSINNTIMGEMKNCSFNTTTEIRDKEKKEYALFYKPDVVPLNETSNTSEYRLINCNTSAVTQACPKVTFEPIPIHYCAPAGYAILKCNNKTFNGTGPCSNVSTVQCTHGIRPVVSTQLLLNGSLAEKEIVIRSENLTNNAKIIIVHLNTSVEIVCTRPGNNTRKSVRIGPGQWFYATGDIIGDIRQAHCNISEGQWNKTLHEVSKELQKHFPNKTIKYERSAGGDMEIATHSFNCGGEFFYCNTSNLFNGTYNGTYINTSSTSYITLQCRIKQIINMWQGVGRAMYAPPIAGNITCKSNITGLLLTRDGGTKNNSNEETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 251 - protein, 652 aa, synthetic construct
MPMGSLQPLATLYLLGMLVASVLAAENLWVTVYYGVPVWKEAKTTLFCASDAKAYEKEVHNVWATHACVPTDPSPQELVLKNVTENFNMWKNDMVDQMHEDIISLWDQSLKPCVKLTPLCVTLNCSNARSNVNVTSINNTIMGEMKNCSFNTTTEIRDKEKKEYALFYKPDVVPLNETSNTSEYRLINCNTSACTQACPKVTFEPIPIHYCAPAGYAILKCNNKTFNGTGPCSNVSTVQCTHGIRPVVSTQLLLNGSLAEKEIVIRSENLTNNAKIIIVHLNTSVEIVCTRPGNNTRKSVRIGPGQTFYATGDIIGDIRQAHCNISEGQWNKTLHEVSKELQKHFPNKTIKYERSAGGRMEIATHSFNCGGEFFYCNTSNLFNGTYNGTYINTSSTSYITLQCRIKQIINMWQGVGRCMYAPPIAGNITCKSNITGLLLTRDGGTKNNSNEETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 252 - protein, 658 aa, synthetic construct
MPMGSLQPLATLYLLGMLVASVLAAENLWVTVYYGVPVWKEAKTTLFCASDAKAYEKEVHNVWATHACVPTDPSPQELVLKNVTENFNMWKNDMVDQMHEDIISLWDQSLKPCVKLTPLCVTLNCSNARSNVNVTSINNTIMGEMKNCSFNTTTEIRDKEKKEYALFYKPDVVPLNETSNTSEYRLINCNTSACTQACPKVTFEPIPIHYCAPAGYAILKCNNKTFNGTGPCSNVSTVQCTHGIRPVVSTQLLLNGSLAEKEIVIRSENLTNNAKIIIVHLNTSVEIVCTRPGNNTRKSVRIGPGQTFYATGDIIGDIRQAHCNISEGQWNKTLHEVSKELQKHFPNKTIKYERSAGGDMEIATHSFNCGGEFFYCNTSNLFNGTYNGTYINTSSTSYITLQCRIKQIINMWQGVGRCMYAPPIAGNITCKSNITGLLLTRDGGTKNNSNEETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDLPETGG

SEQ ID NO: 253 - protein, 820 aa, synthetic construct
MPMGSLQPLATLYLLGMLVASVLAAENLWVTVYYGVPVWKEAKTTLFCASDAKAYEKEVHNVWATHACVPTDPSPQELVLKNVTENFNMWKNDMVDQMHEDIISLWDQSLKPCVKLTPLCVTLNCSNARSNVNVTSINNTIMGEMKNCSFNTTTEIRDKEKKEYALFYKPDVVPLNETSNTSEYRLINCNTSACTQACPKVTFEPIPIHYCAPAGYAILKCNNKTFNGTGPCSNVSTVQCTHGIRPVVSTQLLLNGSLAEKEIVIRSENLTNNAKIIIVHLNTSVEIVCTRPGNNTRKSVRIGPGQTFYATGDIIGDIRQAHCNISEGQWNKTLHEVSKELQKHFPNKTIKYERSAGGDMEIATHSFNCGGEFFYCNTSNLFNGTYNGTYINTSSTSYITLQCRIKQIINMWQGVGRCMYAPPIAGNITCKSNITGLLLTRDGGTKNNSNEETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 254 - protein, 662 aa, synthetic construct
MPMGSLQPLATLYLLGMLVASVLAAENLWVTVYYGVPVWKEAKTTLFCASDAKAYKKEVHNVWATHACVPTDPSPQELFLKNVTENFNMWKNDMVDQMHEDIISLWDQSLKPCVKLTPLCVTLICSNAIVKNSTTEEMSTALVKNSTTEAMKNCSFNTTTEIRDKEKKEYALFYRPDIVPLNNETGNISEYRLINCNTSACTQACPKVTFEPIPIHYCAPAGYAILKCNDETFNGTGPCSNVSTVQCTHGIRPVVSTQLLLNGSLAKEEIVIRSENLTNNAKIIIVHLHTPVEIVCTRPNNNTRKSVRIGPGQTFYATGDIIGDPRKAHCNISEKDWNKTLQEVGKELQKHFPNKTIRYNQSAGGDMEITTHSFNCGGEFFYCNTSKLFNSTYNDTYISTNSSANNSSTITLQCRIKQIINMWQGVGRCMYAPPIAGNITCKSNITGLLLTRDGGPDSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 255 - protein, 662 aa, synthetic construct
MPMGSLQPLATLYLLGMLVASVLAAENLWVTVYYGVPVWKEAKTTLFCASDAKAYKKKVHNVWATHACVPTDPSPQELFLKNVTENFNMWKNDMVDQMHEDIISLWDQSLKPCVKLTPLCVTLICSNAIVKNSTTEEMSTALVKNSTTEAMKNCSFNTTTEIRDKEKKEYALFYRPDIVPLNNETGNISEYRLINCNTSAVTQACPKVTFEPIPIHYCAPAGYAILKCNDETFNGTGPCSNVSTVQCTHGIRPVVSTQLLLNGSLAKEEIVIRSENLTNNAKIIIVHLHTPVEIVCTRPNNNTRKSVRIGPGQWFYATGDIIGDPRKAHCNISEKDWNKTLQEVGKELQKHFPNKTIRYNQSAGGDMEITTHSFNCGGEFFYCNTSKLFNSTYNDTYISTNSSANNSSTITLQCRIKQIINMWQGVGRAMYAPPIAGNITCKSNITGLLLTRDGGPDSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 256 - protein, 662 aa, synthetic construct
MPMGSLQPLATLYLLGMLVASVLAAENLWVTVYYGVPVWKEAKTTLFCASDAKAYKKEVHNVWATHACVPTDPSPQELFLKNVTENFNMWKNDMVDQMHEDIISLWDQSLKPCVKLTPLCVTLICSNAIVKNSTTEEMSTALVKNSTTEAMKNCSFNTTTEIRDKEKKEYALFYRPDIVPLNNETGNISEYRLINCNTSACTQACPKVTFEPIPIHYCAPAGYAILKCNDETFNGTGPCSNVSTVQCTHGIRPVVSTQLLLNGSLAKEEIVIRSENLTNNAKIIIVHLHTPVEIVCTRPNNNTRKSVRIGPGQTFYATGDIIGDPRKAHCNISEKDWNKTLQEVGKELQKHFPNKTIRYNQSAGGRMEITTHSFNCGGEFFYCNTSKLFNSTYNDTYISTNSSANNSSTITLQCRIKQIINMWQGVGRCMYAPPIAGNITCKSNITGLLLTRDGGPDSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 257 - protein, 668 aa, synthetic construct
MPMGSLQPLATLYLLGMLVASVLAAENLWVTVYYGVPVWKEAKTTLFCASDAKAYKKEVHNVWATHACVPTDPSPQELFLKNVTENFNMWKNDMVDQMHEDIISLWDQSLKPCVKLTPLCVTLICSNAIVKNSTTEEMSTALVKNSTTEAMKNCSFNTTTEIRDKEKKEYALFYRPDIVPLNNETGNISEYRLINCNTSACTQACPKVTFEPIPIHYCAPAGYAILKCNDETFNGTGPCSNVSTVQCTHGIRPVVSTQLLLNGSLAKEEIVIRSENLTNNAKIIIVHLHTPVEIVCTRPNNNTRKSVRIGPGQTFYATGDIIGDPRKAHCNISEKDWNKTLQEVGKELQKHFPNKTIRYNQSAGGDMEITTHSFNCGGEFFYCNTSKLFNSTYNDTYISTNSSANNSSTITLQCRIKQIINMWQGVGRCMYAPPIAGNITCKSNITGLLLTRDGGPDSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDLPETGG

SEQ ID NO: 258 - protein, 830 aa, synthetic construct
MPMGSLQPLATLYLLGMLVASVLAAENLWVTVYYGVPVWKEAKTTLFCASDAKAYKKEVHNVWATHACVPTDPSPQELFLKNVTENFNMWKNDMVDQMHEDIISLWDQSLKPCVKLTPLCVTLICSNAIVKNSTTEEMSTALVKNSTTEAMKNCSFNTTTEIRDKEKKEYALFYRPDIVPLNNETGNISEYRLINCNTSACTQACPKVTFEPIPIHYCAPAGYAILKCNDETFNGTGPCSNVSTVQCTHGIRPVVSTQLLLNGSLAKEEIVIRSENLTNNAKIIIVHLHTPVEIVCTRPNNNTRKSVRIGPGQTFYATGDIIGDPRKAHCNISEKDWNKTLQEVGKELQKHFPNKTIRYNQSAGGDMEITTHSFNCGGEFFYCNTSKLFNSTYNDTYISTNSSANNSSTITLQCRIKQIINMWQGVGRCMYAPPIAGNITCKSNITGLLLTRDGGPDSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 259 - DNA, 1952 bp, synthetic construct
  source 1..1952: mol_type = other DNA
atgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgcccgcgcctacgagaaggaggtgcacaacgtgtgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgctccgacgccggctccggcggcgtggaggagatgaagaactgctccttcaacaccaccaccgagatccgcgacaaggagaagaaggagtacgccctgttctacaagcccgacatcgtgcccctgtccgagaccaacaacacctccgagtaccgcctgatcaactgcaacacctccgcctgcacccaggcctgccccaaggtgaccttcgagcccatccccatccactactgcgcccccgccggctacgccatcctgaagtgcaacgacgagaccttcaacggcaccggcccctgctccaacgtgtccaccgtgcagtgcacccacggcatccgccccgtggtgtccacccagctgctgctgaacggctccctggccgagaaggagatcgtgatccgctccgagaacctgaccaacaacgccaagatcatcatcgtgcacctgcacacccccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgctgcatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggactgataagcggccgcggatcc

SEQ ID NO: 260 - protein, 644 aa, synthetic construct
MGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGSGGVEEMKNCSFNTTTEIRDKEKKEYALFYKPDIVPLSETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 261 - protein, 622 aa, synthetic construct
AENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGSGGVEEMKNCSFNTTTEIRDKEKKEYALFYKPDIVPLSETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 262 - DNA, 1952 bp, synthetic construct; identical to SEQ ID NO: 259
  source 1..1952: mol_type = other DNA

SEQ ID NO: 263 - DNA, 1944 bp, synthetic construct
  source 1..1944: mol_type = other DNA
atgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccggctccggcggcgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggattgatga

SEQ ID NO: 264 - DNA, 2448 bp, synthetic construct
  source 1..2448: mol_type = other DNA
atgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccggctccggcggcgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaaacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggattctggcggagacattatcaagctgctgaacgagcaagtgaacaaagaaatgcagtcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtccggctcctgatga

SEQ ID NO: 265 - DNA, 2448 bp, synthetic construct
  source 1..2448: mol_type = other DNA
atgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccggcaacggctccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaaacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggattctggcggagacattatcaagctgctgaacgagcaagtgaacaaagaaatgcagtcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtccggctcctgatga

SEQ ID NO: 266 - DNA, 2448 bp, synthetic construct
  source 1..2448: mol_type = other DNA
atgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccggctccggcggcgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaaacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccgcctccaccagcaccatcactctgcagtgccggatcaagcagatcatcaatatgtggcaaggcgtcggccggtgtatgtacgcccctcctatcgccggcgccatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggattctggcggagacattatcaagctgctgaacgagcaagtgaacaaagaaatgcagtcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtccggctcctgatga

SEQ ID NO: 267 - DNA, 2448 bp, synthetic construct
  source 1..2448: mol_type = other DNA
atgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccggcaacggctccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaaacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccgcctccaccagcaccatcactctgcagtgccggatcaagcagatcatcaatatgtggcaaggcgtcggccggtgtatgtacgcccctcctatcgccggcgccatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggattctggcggagacattatcaagctgctgaacgagcaagtgaacaaagaaatgcagtcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtccggctcctgatga

SEQ ID NO: 268 - DNA, 2022 bp, synthetic construct
  source 1..2022: mol_type = other DNA
atgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccggctccggcggcgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggcctggtgcctcagcagagcggcggcctgaacgacatcttcgaggcccagaagatcgagtggcacgaaggctgatga

SEQ ID NO: 269 - DNA, 1944 bp, synthetic construct
  source 1..1944: mol_type = other DNA
atgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccggcaacggctccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggattgatga

SEQ ID NO: 270 - DNA, 2022 bp, synthetic construct
  source 1..2022: mol_type = other DNA
atgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccggcaacggctccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggcctggtgcctcagcagagcggcggcctgaacgacatcttcgaggcccagaagatcgagtggcacgaaggctgatga

SEQ ID NO: 271 - DNA, 2082 bp, synthetic construct
  source 1..2082: mol_type = other DNA
atgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgcccgcgcctacgagaaggaggtgcacaacgtgtgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgctccgacgccggctccggcggcgtggaggagatgaagaactgctccttcaacaccaccaccgagatccgcgacaaggagaagaaggagtacgccctgttctacaagcccgacatcgtgcccctgtccgagaccaacaacacctccgagtaccgcctgatcaactgcaacacctccgcctgcacccaggcctgccccaaggtgaccttcgagcccatccccatccactactgcgcccccgccggctacgccatcctgaagtgcaacgacgagaccttcaacggcaccggcccctgctccaacgtgtccaccgtgcagtgcacccacggcatccgccccgtggtgtccacccagctgctgctgaacggctccctggccgagaaggagatcgtgatccgctccgagaacctgaccaacaacgccaagatcatcatcgtgcacctgcacacccccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgctgcatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacctggaggtgctgttccagggcccaggccatcaccaccatcaccaccatcatagcgcctggtcccacccccagttcgagaagggcggcggtagtggagggggcggatctggcggctcagcttggagccacccccagttcgaaaagtgataa

SEQ ID NO: 272 - DNA, 2016 bp, synthetic construct
  source 1..2016: mol_type = other DNA
atgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgcccgcgcctacgagaaggaggtgcacaacgtgtgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgctccgacgccggctccggcggcgtggaggagatgaagaactgctccttcaacaccaccaccgagatccgcgacaaggagaagaaggagtacgccctgttctacaagcccgacatcgtgcccctgtccgagaccaacaacacctccgagtaccgcctgatcaactgcaacacctccgcctgcacccaggcctgccccaaggtgaccttcgagcccatccccatccactactgcgcccccgccggctacgccatcctgaagtgcaacgacgagaccttcaacggcaccggcccctgctccaacgtgtccaccgtgcagtgcacccacggcatccgccccgtggtgtccacccagctgctgctgaacggctccctggccgagaaggagatcgtgatccgctccgagaacctgaccaacaacgccaagatcatcatcgtgcacctgcacacccccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgctgcatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacggaggaggcctggtgccacagcagagcggaggcctgaacgacatcttcgaggcccagaagatcgagtggcacgagggctgataa

SEQ ID NO: 273 - DNA, 2016 bp, synthetic construct
  source 1..2016: mol_type = other DNA
atgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgcccgcgcctacgagaaggaggtgcacaacgtgtgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgctccgacgccggcaacggcagcgtggaggagatgaagaactgctccttcaacaccaccaccgagatccgcgacaaggagaagaaggagtacgccctgttctacaagcccgacatcgtgcccctgtccgagaccaacaacacctccgagtaccgcctgatcaactgcaacacctccgcctgcacccaggcctgccccaaggtgaccttcgagcccatccccatccactactgcgcccccgccggctacgccatcctgaagtgcaacgacgagaccttcaacggcaccggcccctgctccaacgtgtccaccgtgcagtgcacccacggcatccgccccgtggtgtccacccagctgctgctgaacggctccctggccgagaaggagatcgtgatccgctccgagaacctgaccaacaacgccaagatcatcatcgtgcacctgcacacccccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgctgcatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacggaggaggcctggtgccacagcagagcggaggcctgaacgacatcttcgaggcccagaagatcgagtggcacgagggctgataa

SEQ ID NO: 274 - DNA, 2082 bp, synthetic construct
  source 1..2082: mol_type = other DNA
atgggctccctgcagcccctggccaccctgtacctgctgggcatgctggtggcctccgtgctggccgccgagaacctgtgggtgaccgtgtactacggcgtgcccgtgtggaaggaggccaagaccaccctgttctgcgcctccgacgcccgcgcctacgagaaggaggtgcacaacgtgtgggccacccacgcctgcgtgcccaccgacccctccccccaggagctggtgctgggcaacgtgaccgagaacttcaacatgtggaagaacgacatggtggaccagatgcacgaggacatcatctccctgtgggaccagtccctgaagccctgcgtgaagctgacccccctgtgcgtgaccctgatctgctccgacgccggcaacggcagcgtggaggagatgaagaactgctccttcaacaccaccaccgagatccgcgacaaggagaagaaggagtacgccctgttctacaagcccgacatcgtgcccctgtccgagaccaacaacacctccgagtaccgcctgatcaactgcaacacctccgcctgcacccaggcctgccccaaggtgaccttcgagcccatccccatccactactgcgcccccgccggctacgccatcctgaagtgcaacgacgagaccttcaacggcaccggcccctgctccaacgtgtccaccgtgcagtgcacccacggcatccgccccgtggtgtccacccagctgctgctgaacggctccctggccgagaaggagatcgtgatccgctccgagaacctgaccaacaacgccaagatcatcatcgtgcacctgcacacccccgtggagatcgtgtgcacccgccccaacaacaacacccgcaagtccgtgcgcatcggccccggccagaccttctacgccaccggcgacatcatcggcgacatcaagcaggcccactgcaacatctccgaggagaagtggaacgacaccctgcagaaggtgggcatcgagctgcagaagcacttccccaacaagaccatcaagtacaaccagtccgccggcggcgacatggagatcaccacccactccttcaactgcggcggcgagttcttctactgcaacacctccaacctgttcaacggcacctacaacggcacctacatctccaccaactcctccgccaactccacctccaccatcaccctgcagtgccgcatcaagcagatcatcaacatgtggcagggcgtgggccgctgcatgtacgccccccccatcgccggcaacatcacctgccgctccaacatcaccggcctgctgctgacccgcgacggcggcaccaactccaacgagaccgagaccttccgccccgccggcggcgacatgcgcgacaactggcgctccgagctgtacaagtacaaggtggtgaagatcgagcccctgggcgtggcccccacccgctgcaagcgccgcgtggtgggccgccgccgccgccgccgcgccgtgggcatcggcgccgtgttcctgggcttcctgggcgccgccggctccaccatgggcgccgcctccatgaccctgaccgtgcaggcccgcaacctgctgtccggcatcgtgcagcagcagtccaacctgctgcgcgcccccgaggcccagcagcacctgctgaagctgaccgtgtggggcatcaagcagctgcaggcccgcgtgctggccgtggagcgctacctgcgcgaccagcagctgctgggcatctggggctgctccggcaagctgatctgctgcaccaacgtgccctggaactcctcctggtccaaccgcaacctgtccgagatctgggacaacatgacctggctgcagtgggacaaggagatctccaactacacccagatcatctacggcctgctggaggagtcccagaaccagcaggagaagaacgagcaggacctgctggccctggacctggaggtgctgttccagggcccaggccatcaccaccatcaccaccatcatagcgcctggtcccacccccagttcgagaagggcggcggtagtggagggggcggatctggcggctcagcttggagccacccccagttcgaaaagtgataa

SEQ ID NO: 275 - protein, 644 aa, synthetic construct; identical to SEQ ID NO: 260

SEQ ID NO: 276 - protein, 622 aa, synthetic construct; identical to SEQ ID NO: 261

SEQ ID NO: 277 - protein, 646 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGSGGVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 278 - protein, 814 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGSGGVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 279 - protein, 814 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGNGSVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 280 - protein, 814 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGNGSVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSAASTSTITLQCRIKQIINMWQGVGRCMYAPPIAGAITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 281 - protein, 672 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGSGGVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGLVPQQSGGLNDIFEAQKIEWHEG

SEQ ID NO: 282 - protein, 646 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGNGSVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 283 - protein, 672 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGNGSVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGLVPQQSGGLNDIFEAQKIEWHEG

SEQ ID NO: 284 - protein, 692 aa, synthetic construct
MGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGSGGVEEMKNCSFNTTTEIRDKEKKEYALFYKPDIVPLSETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDLEVLFQGPGHHHHHHHHSAWSHPQFEKGGGSGGGGSGGSAWSHPQFEK

SEQ ID NO: 285 - protein, 670 aa, synthetic construct
MGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGSGGVEEMKNCSFNTTTEIRDKEKKEYALFYKPDIVPLSETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGLVPQQSGGLNDIFEAQKIEWHEG

SEQ ID NO: 286 - protein, 670 aa, synthetic construct
MGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGNGSVEEMKNCSFNTTTEIRDKEKKEYALFYKPDIVPLSETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGLVPQQSGGLNDIFEAQKIEWHEG

SEQ ID NO: 287 - protein, 692 aa, synthetic construct
MGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDAGNGSVEEMKNCSFNTTTEIRDKEKKEYALFYKPDIVPLSETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDLEVLFQGPGHHHHHHHHSAWSHPQFEKGGGSGGGGSGGSAWSHPQFEK

SEQ ID NO: 288 - protein, 849 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRGMMRGEIKNCSFNTTTEIRDKKKKEYALFYKPDIVPLSETNNTSEYRLINCNTSACTQVCPKLTFEPIPIHYCAPAGYAILKCNDETFNGTGPCSNVSTVQCTHGIRPVLSTQLLLNGSLAEKEIVIRSENLTNNAKIIIVHLHTPVEIVCTRPNNNTRKSVRIGPGQTFYATGDIIGDIKMAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDSWNSLWNWFSITKWLWYIKIFIMIVGGLIGLRIVFAVLSIVNRVRQGYSPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 289 - protein, 849 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRGMMRGEIKNCSFNTTTEIRDKKKKEYALFYKPDIVPLSETNNTSEYRLINCNTSACTQVCPKLTFEPIPIHYCAPAGYAILKCNDETFNGTGPCSNVSTVQCTHGIRPVLSTQLLLNGSLAEKEIVIRSENLTNNAKIIIVHLHTPVEIVCTRPNNNTRKSVRIGPGQTFYATGDIIGDIKMAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 290 - protein, 741 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRGMMRGEIKNCSFNTTTEIRDKKKKEYALFYKPDIVPLSETNNTSEYRLINCNTSACTQVCPKLTFEPIPIHYCAPAGYAILKCNDETFNGTGPCSNVSTVQCTHGIRPVLSTQLLLNGSLAEKEIVIRSENLTNNAKIIIVHLHTPVEIVCTRPNNNTRKSVRIGPGQTFYATGDIIGDIKMAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPI

SEQ ID NO: 291 - protein, 698 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRGMMRGEIKNCSFNTTTEIRDKKKKEYALFYKPDIVPLSETNNTSEYRLINCNTSACTQVCPKLTFEPIPIHYCAPAGYAILKCNDETFNGTGPCSNVSTVQCTHGIRPVLSTQLLLNGSLAEKEIVIRSENLTNNAKIIIVHLHTPVEIVCTRPNNNTRKSVRIGPGQTFYATGDIIGDIKMAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDSWNSLWNWFSITKWLWYIKIFIMIVGGLIGLRIVFAVLSIVNRVRQGI

SEQ ID NO: 292 - protein, 709 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRGMMRGEIKNCSFNTTTEIRDKKKKEYALFYKPDIVPLSETNNTSEYRLINCNTSACTQVCPKLTFEPIPIHYCAPAGYAILKCNDETFNGTGPCSNVSTVQCTHGIRPVLSTQLLLNGSLAEKEIVIRSENLTNNAKIIIVHLHTPVEIVCTRPNNNTRKSVRIGPGQTFYATGDIIGDIKMAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDSWNSLWNWFSITKWLWYIKIFIMIVGGLIGLRIVFAVLSIVNRVRQGIRPVFSSPPSYF

SEQ ID NO: 293 - protein, 741 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRGMMRGEIKNCSFNTTTEIRDKKKKEYALFYKPDIVPLSETNNTSEYRLINCNTSACTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKLVLIGPGQWFYATGDIIGDIKMAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPI

SEQ ID NO: 294 - protein, 698 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRGMMRGEIKNCSFNTTTEIRDKKKKEYALFYKPDIVPLSETNNTSEYRLINCNTSACTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKLVLIGPGQWFYATGDIIGDIKMAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDSWNSLWNWFSITKWLWYIKIFIMIVGGLIGLRIVFAVLSIVNRVRQGI

SEQ ID NO: 295 - protein, 709 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRGMMRGEIKNCSFNTTTEIRDKKKKEYALFYKPDIVPLSETNNTSEYRLINCNTSACTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKLVLIGPGQWFYATGDIIGDIKMAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDSWNSLWNWFSITKWLWYIKIFIMIVGGLIGLRIVFAVLSIVNRVRQGIRPVFSSPPSYF

SEQ ID NO: 296 - protein, 698 aa, synthetic construct; identical to SEQ ID NO: 291

SEQ ID NO: 297 - protein, 849 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRGMMRGEIKNCSFNTTTEIRDKKKKEYALFYKPDIVPLSETNNTSEYRLINCNTSACTQVCPKLTFEPIPIHYCAPAGYAILKCNDETFNGTGPCSNVSTVQCTHGIRPVLSTQLLLNGSLAEKEIVIRSENLTNNAKIIIVHLHTPVEIVCTRPNNNTRKLVLIGPGQWFYATGDIIGDIKMAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASAA

SEQ ID NO: 298 - protein, 741 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRGMMRGEIKNCSFNTTTEIRDKKKKEYALFYKPDIVPLSETNNTSEYRLINCNTSACTQVCPKLTFEPIPIHYCAPAGYAILKCNDETFNGTGPCSNVSTVQCTHGIRPVLSTQLLLNGSLAEKEIVIRSENLTNNAKIIIVHLHTPVEIVCTRPNNNTRKLVLIGPGQWFYATGDIIGDIKMAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPI

SEQ ID NO: 299 - protein, 698 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRGMMRGEIKNCSFNTTTEIRDKKKKEYALFYKPDIVPLSETNNTSEYRLINCNTSACTQVCPKLTFEPIPIHYCAPAGYAILKCNDETFNGTGPCSNVSTVQCTHGIRPVLSTQLLLNGSLAEKEIVIRSENLTNNAKIIIVHLHTPVEIVCTRPNNNTRKLVLIGPGQWFYATGDIIGDIKMAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDSWNSLWNWFSITKWLWYIKIFIMIVGGLIGLRIVFAVLSIVNRVRQGI

SEQ ID NO: 300 - protein, 709 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRGMMRGEIKNCSFNTTTEIRDKKKKEYALFYKPDIVPLSETNNTSEYRLINCNTSACTQVCPKLTFEPIPIHYCAPAGYAILKCNDETFNGTGPCSNVSTVQCTHGIRPVLSTQLLLNGSLAEKEIVIRSENLTNNAKIIIVHLHTPVEIVCTRPNNNTRKLVLIGPGQWFYATGDIIGDIKMAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDSWNSLWNWFSITKWLWYIKIFIMIVGGLIGLRIVFAVLSIVNRVRQGIRPVFSSPPSYF

SEQ ID NO: 301 - protein, 839 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRGMMRGEIKNCSFNTTTEIRDKKKKEYALFYKPDIVPLSETNNTSEYRLINCNTSACTQVCPKLTFEPIPIHYCAPAGYAILKCNDETFNGTGPCSNVSTVQCTHGIRPVLSTQLLLNGSLAEKEIVIRSENLTNNAKIIIVHLHTPVEIVCTRPNNNTRKLVLIGPGQWFYATGDIIGDIKMAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDGSGGSGGSGGSGGSEKAAKAEEAARKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 302 - protein, 827 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRGMMRGEIKNCSFNTTTEIRDKKKKEYALFYKPDIVPLSETNNTSEYRLINCNTSACTQVCPKLTFEPIPIHYCAPAGYAILKCNDETFNGTGPCSNVSTVQCTHGIRPVLSTQLLLNGSLAEKEIVIRSENLTNNAKIIIVHLHTPVEIVCTRPNNNTRKLVLIGPGQWFYATGDIIGDIKMAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDGGSEKAAKAEEAARDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 303 - protein, 823 aa, synthetic construct
MPMGSLQPLATLYLLGMLVASVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICTNYAPKLRGMMRGEIKNCSFNTTTEIRDKKKKEYALFYKPDIVPLSETNNTSEYRLINCNTSACTQVCPKLTFEPIPIHYCAPAGYAILKCNDETFNGTGPCSNVSTVQCTHGIRPVLSTQLLLNGSLAEKEIVIRSENLTNNAKIIIVHLHTPVEIVCTRPNNNTRKLVLIGPGQWFYATGDIIGDIKMAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDGGGLVPQQSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

(19) United States
(12) Patent Application Publication
(10) Pub. No.: US 2026/0217769 A1
(43) Pub. Date: Jul. 30, 2026

(54) COMPOSITIONS COMPRISING HIV-1 ENVELOPES WITH ENGINEERED V1V2 OR MRNAS ENCODING THE SAME FOR BROAD V3-GLYCAN NEUTRALIZING ANTIBODY BINDING

(71) Applicants: Duke University, Durham, NC (US); The Trustees of The University of Pennsylvania, Philadelphia, PA (US)

(72) Inventors: Kevin O. SAUNDERS, Durham, NC (US); Barton F. HAYNES, Durham, NC (US); Daniel WRAPP, Durham, NC (US); Drew WEISSMAN, Philadelphia, PA (US)

(21) Appl. No.: 19/124,194

(22) PCT Filed: Oct. 25, 2023
(86) PCT No.: PCT/US2023/077804

§ 371 Date: Apr. 24, 2025

Related U.S. Application Data
(60) Provisional application No. 63/419,264, filed on Oct. 25, 2022.

(51) Int. Cl. C07K 14/005; A61K 39/00; A61K 39/21

(52) U.S. Cl. CPC C07K 14/005; A61K 39/21; A61K 2039/53; A61K 2039/545; A61K 2039/55555; C12N 2740/16122; C12N 2740/16134

(57) ABSTRACT

The invention is directed to modified HIV-1 envelopes, compositions comprising these modified envelopes, nucleic acids encoding these modified envelopes, compositions comprising these nucleic acids, and methods of using these modified HIV-1 envelopes and/or these nucleic acids to induce immune responses.

This International Patent application claims the benefit of and priority to U.S. Application No. 63/419,264, filed Oct. 25, 2022, entitled “COMPOSITIONS COMPRISING HIV-1 ENVELOPES WITH ENGINEERED VIV2 OR MRNAS ENCODING THE SAME FOR BROAD V3-GLYCAN NEUTRALIZING ANTIBODY BINDING,” the content of which is hereby incorporated by reference in its entirety.

This invention was made with government support from the NIH, NIAID, Division of AIDS for UMI grant AI144371 for the Consortium for HIV/AIDS Vaccine Development (CHAVD). The government has certain rights in the invention.

### TECHNICAL FIELD

The present invention relates in general, to a composition suitable for use in inducing anti-HIV-1 antibodies, and, in particular, to immunogenic compositions comprising envelope proteins and nucleic acids to induce cross-reactive neutralizing antibodies and increase their breadth of coverage. The invention also relates to methods of inducing such broadly neutralizing anti-HIV-1 antibodies using such compositions.

### BACKGROUND

The development of a safe and effective HIV-1 vaccine is one of the highest priorities of the scientific community working on the HIV-1 epidemic. While anti-retroviral treatment (ART) has dramatically prolonged the lives of HIV-1 infected patients, ART is not routinely available in developing countries.

### SUMMARY OF THE INVENTION

In certain embodiments, the invention provides compositions and methods for induction of an immune response, for example cross-reactive (broadly) neutralizing (bn) Ab induction.

In certain aspects the invention provides a recombinant protein or nucleic acid encoding a recombinant protein as described in Tables 5 or 6. In certain aspects the invention provides a selection of HIV-1 envelopes for use as prime and boost immunogens in methods to induce HIV-1 neutralizing antibodies. In certain aspects, the invention provides a selection of HIV-1 envelopes for use as a boost immunogen in methods to induce HIV-1 neutralizing antibodies. In certain aspects, the invention provides a selection of HIV-1 envelopes for use as a prime immunogen in methods to induce HIV-1 neutralizing antibodies.

In certain embodiments, the invention provides a recombinant HIV-1 envelope protein or nucleic acid encoding a recombinant HIV-1 envelope protein as described in Tables 5 or 6. In certain aspects, the invention provides a recombinant HIV-1 envelope sequence or nucleic acid encoding a recombinant protein HIV-1 envelope sequence comprising residues 132-154 (HXB2 numbering) of the N332-GT5 V1 loop. (See Example 1, Table 5 and FIGS. 38-39 and 52). In some embodiments, the recombinant HIV-1 envelope protein is
CH0848.d949.10.17_MD64V1_E169K_D230N_H289N_P291S_F14_Y7121_gp160_CD5ss (SEQ ID NO: 19). In some embodiments, the recombinant HIV-1 envelope protein is CH0848.d949.10.17_MD64V1_E169K_D230N_H289N_P291S_F14_Y712I_SOSL_GS.PC_gp160_CD5ss (SEQ ID NO: 23). In some embodiments, the recombinant HIV-1 envelope protein is
CH0848.d949.10.17_VIswap_E169K_S148G_Q328M_F14_DS_SOSIP_MD2_MD33_Y71 2I_gp150.755_CDSss (SEQ ID NO: 290). In some embodiments, the recombinant HIV-1 envelope protein is
CH0848.d949.10.17_Vlswap_E169K_S148G_Q328M_F14_DS_SOSIP_MD2_MD33_Y71 2I_gp150.712_CD5ss (SEQ ID NO: 291).

In certain aspects, the residues 132-154 of the N332-GT5 V1 loop can be incorporated into any HIV-1 envelope sequences from the CH848 infected individual and variants thereof. See e.g., US2020/0113997 incorporated herein by reference in its entirety including FIGS. 40A-C, 41A-41C, 44A-D, 45, 46, 47A, 49A-B, SOA-D, 51, 52A-B, 53A, 53D, 54A-F, 77A-L, and 78A-B and SEQ ID NOs disclosed therein. In some embodiments, the residues 132-154 of the N332-GT5 V1 loop can be incorporated into envelope CH848.3.D0949.10.17 (also referred to as CH848.d0949.10.17WT) and variants thereof, including, but not limited to, CH848.d0949.10.17 DT (also referred to as CH848.d0949.10.17.N133D.N138T). In some embodiments, the residues 132-154 of the N332-GT5 V1 loop can be incorporated into envelope CH848.d0808.15.15 and variants thereof. In some embodiments, the residues 132-154 of the N332-GT5 V1 loop can be incorporated into envelope CH848.d0358.80.06 and variants thereof. In some embodiments, the residues 132-154 of the N332-GT5 V1 loop can be incorporated into envelope CH848.d1432.5.41 and variants thereof. In some embodiments, the residues 132-154 of the N332-GT5 V1 loop can be incorporated into envelope CH848.d1621.4.44 and variants thereof. In some embodiments, the residues 132-154 of the N332-GT5 V1 loop can be incorporated into envelope CH848.d1305.10.35 and variants thereof.

In certain aspects the invention provides a selection of a series of immunogens and immunogen designs for induction of neutralizing HIV-1 antibodies, e.g. but not limited to V3 glycan epitope targeting antibodies, the selection comprising envelopes as follows: 1) CH848.d0949.10.17 DT (also referred to as CH848.d0949.10.17.N133D.N138T), 2) CH848.d0949.10.17 (also referred to as CH848.d0949.10.17WT), 3) CH848.d0808.15.15, 4) CH848.d0358.80.06, 5) CH848.d1432.5.41, 6) CH848.d1621.4.44 and 7) CH848.d1305.10.35 (see Tables 3 and 4). In some embodiments, the selection further comprises any HIV-1 envelope sequence as described in Tables 5 or 6. In some embodiments, the selection further comprises any HIV-1 envelope sequence with the modification to the V1 loop described herein. In some embodiments the selection comprises additional HIV-1 Envs, P0402.c2.11 and ZM246F. In some embodiments, the HIV-1 envelope protein sequence comprises

CH0848.d949.10.17_MD64V1_E169K_D230N_H289N_P291S_F14_Y712I_gp160_CD5ss (SEQ ID NO: 19). In some embodiments, the HIV-1 envelope protein sequence comprises CH0848.d949.10.17_MD64V1_E169K_D230N_H289N_P291S_F14_Y712I_SOSL_GS.PC_gp160_CD5ss (SEQ ID NO: 23). In some embodiments, the HIV-1 envelope protein sequence comprises
CH0848.d949.10.17_V1swap_E169K_S148G_Q328M_F14_DS_SOSIP_MD2_MD33_Y71 2I_gp150.755_CD5ss (SEQ ID NO: 290). In some embodiments, the HIV-1 envelope protein sequence comprises
CH0848.d949.10.17_V1swap_E169K_S148G_Q328M_F14 DS_SOSIP_MD2_MD33_Y71 2I_gp150.712_CDSss (SEQ ID NO: 291).

In certain aspects the invention provides a selection of a series of immunogens and immunogen designs for induction of neutralizing HIV-1 antibodies, e.g. but not limited to V3 glycan epitope targeting antibodies, the selection comprising envelopes as follows: 1) CH848.d0949.10.17 DT (also referred to as CH848.d0949.10.17.N133D.N138T), 2) CH848.d0949.10.17 (also referred to as CH848.d0949.10.17WT), 3) CH848.d0808.15.15, 4) CH848.d0358.80.06, 5) CH848.d1432.5.41, 6) CH848.d1621.4.44, 7) CH848.d1305.10.35, (see Tables 3 and 4) and 8) any HIV-1 envelope sequence as described in Tables 5 or 6 or any HIV-1 envelope sequence with the modification to the V1 loop described herein. In some embodiments the selection comprises additional HIV-1 Envs, P0402.c2.11 and ZM246F.

In certain embodiments, the methods use compositions comprising HIV-1 envelope immunogens designed to bind to precursors, and/or unmutated common ancestors (UCAs) of different HIV-1 bnAbs. In certain embodiments, these are UCAs of V1V2 glycan and V3 glycan binding antibodies. Thus, in certain embodiments the invention provides HIV-1 envelope immunogen designs with multimerization and variable region sequence optimization for enhanced UCA-targeting. In certain embodiments the invention provides HIV-1 envelope immunogen designs with multimerization and variable region sequence optimization for enhanced targeting and inductions of multiple antibody lineages, e.g. but not limited to V3 lineage, VIV2 lineages of antibodies.

In certain embodiments, the invention provides HIV-1 envelope immunogens designed to bind to more than one unmutated common ancestor (UCA) of different HIV-1 bnAbs. In certain embodiments, the invention provides HIV-1 envelope immunogens designed to bind to one or more of DH270 UCA, BG18 UCA, or BF250 UCA. (See Example 1). In certain embodiments, the invention provides HIV-1 envelope immunogens designed to bind to the UCA of DH270, BG18, and BF250 BnAb lineages.

In certain aspects the invention provides compositions comprising a selection of HIV-1 envelopes and/or nucleic acids encoding these envelopes as described herein for example but not limited to designs as described herein. Without limitations, these selected combinations comprise envelopes which provide representation of the sequence (genetic) and antigenic diversity of the HIV-1 envelope variants which lead to the induction of V1V2 glycan and V3 glycan antibody lineages.

In certain aspects the recombinant HIV-1 envelope optionally comprises any combinations of additional modifications, such as the modifications described in Table 2. In certain aspects the invention provides a recombinant HIV-1 envelope that in addition to comprising residues 132-154 (HXB2 numbering) of the N332-GT5 V1 loop can further lack glycosylation at position N133 and N138 (HXB2 numbering), comprise glycosylation at N301 (HXB2 numbering) and N332 (HXB2 numbering), comprise modifications wherein glycan holes are filled (D230N_H289N_P291S (HXB2 numbering)), comprise the “GDIR” (SEQ ID NO: 1) or “GDIK” motif (SEQ ID NO: 2), or any trimer stabilization modifications, UCA targeting modification, immunogenicity modification, or combinations thereof, for example but not limited to these described in Table 2. In certain embodiments the recombinant envelope optionally comprises any combinations of these modifications.

In certain embodiments, the recombinant HIV-1 envelope binds to precursors, and/or UCAs of different HIV-1 bnAbs. In certain embodiments, these are UCAs of V1V2 glycan and V3 glycan antibodies. In certain embodiments the envelope is 19CV3. In certain embodiments the envelope is any one of the envelopes listed in Table 1 or Table 2. In certain embodiments, the envelope is not CH848 10.17 DT variant described previously in US2020/0113997.

In certain embodiments the envelope is a protomer which could be comprised in a stable trimer.

In certain embodiments the envelope comprises additional mutations stabilizing the envelope trimer. In certain embodiments these include, but are not limited to, SOSIP mutations. In certain embodiments mutations are selected from sets F1-F14, VT1-VT8 mutations described herein, or any combination or subcombination within a set. In certain embodiments, the selected mutations are F14. In other embodiments, the selected mutations are VT8. In certain embodiments, the selected mutations are F14 and VT8 combined.

In certain embodiments, the invention provides a recombinant HIV-1 envelope of Table 5 and/or FIGS. 38-39 and 52. In certain embodiments, the invention provides a nucleic acid encoding any of the recombinant envelopes. In certain embodiments, the invention provides a mRNA of FIG. 40 or 58 or encoding any of the recombinant envelopes of FIG. 52. In certain embodiments, the nucleic acids comprise an mRNA formulated for use as a pharmaceutical composition.

In certain embodiments the inventive designs comprise specific changes (D230N_H289N_P291S (HXB2 numbering)), which fill glycan holes with the introduction of new glycosylation sites to prevent the binding of strain-specific antibodies that could hinder broad neutralizing antibody development (Wagh, Kshitij et al. “Completeness of HIV-1 Envelope Glycan Shield at Transmission Determines Neutralization Breadth.” Cell reports vol. 25,4 (2018): 893-908.e7. doi: 10.1016/j.celrep.2018.09.087; Crooks, Ema T et al. “Vaccine-Elicited Tier 2 HIV-1 Neutralizing Antibodies Bind to Quaternary Epitopes Involving Glycan-Deficient Patches Proximal to the CD4 Binding Site.” PLOS pathogens vol. 11,5 €1004932. 29 May. 2015, doi: 10.1371/journal.ppat. 1004932).

In certain embodiments the inventive designs comprise specific changes E169K (HXB2 numbering). In certain embodiments, CH848.d0949.10.17DT envelope comprises additional modifications D230N.H289N.P291S.E169K and is referred to as CH848.d0949.10.17 Dte. In certain embodiments, CH848.d0949.10.17 envelope comprises additional modifications D230N.H289N.P291S.E169K and is referred to as CH848.d0949.10.17WTe. In certain embodiments, CH848.d0949.10.17DT envelope comprises additional modifications referred to as CH848.0949.10.17DT.GS designs. See Table 2. In certain embodiments, CH848.d0949.10.17DT.GS envelopes comprise additional modifications D230N.H289N.P291S.E169K. See Table 2.

In non-limiting embodiments, the envelope in the selections for immunization are included as trimers. In non-limiting embodiments, the envelope in the selections for immunization are included as trimers and as mRNAs. In non-limiting embodiments, the envelope in the selections for immunization are included as nanoparticles. In non-limiting embodiments, the envelope in the selections for immunization are included as nanoparticles and as mRNAs. In certain embodiments, the mRNAs are also encapsulated in a lipid nanoparticle. The designation scNP refers to a non-limiting embodiment of a protein nanoparticle formed by sortase conjugation reaction. In non-limiting embodiments, nanoparticles comprise fusion proteins, for example ferritin-envelope fusion proteins.

In certain embodiments, the inventive designs comprise modifications, including without limitation fusion of the HIV-1 envelope with ferritin using linkers between the HIV-1 envelope and ferritin designed to optimize ferritin nanoparticle assembly.

In certain embodiments, the invention provides HIV-1 envelopes comprising Lys327 (HXB2 numbering) optimized for administration as a prime to initiate V3 glycan antibody lineage, e.g. DH270 antibody lineage.

In certain embodiments, the invention provides HIV-1 envelopes comprising Lys169 (HXB2 numbering).

In certain embodiments, the invention provides a composition comprising any one of the inventive envelopes, e.g., as disclosed in Tables 5 or 6, or nucleic acid sequences encoding the same. In certain embodiments, the nucleic acid is mRNA. In certain embodiments, the mRNA is comprised in a lipid nano-particle (LNP).

In certain embodiments, the compositions comprise a nucleic acid, wherein the nucleic acid is operably linked to a promoter, and could be inserted in an expression vector. In some embodiments, the expression vector comprises DNA. In certain embodiments, the nucleic acid is a mRNA. In certain embodiments, the nucleic acid is encapsulated in a lipid nanoparticle.

In certain aspects, the invention provides nucleic acids comprising sequences encoding polypeptides or proteins of the invention. In certain embodiments, the nucleic acids are DNAs. In certain embodiments, the nucleic acids are mRNAs. In certain aspects, the invention provides expression vectors comprising the nucleic acids of the invention. In some embodiments, the expression vector comprises DNA.

In some embodiments, the invention provides a nucleic acid of FIG. 40 or 58. In non-limiting embodiments, the nucleic acid is an mRNA. In some embodiments, the mRNA comprises the nucleic acids according to FIG. 40 or 58, wherein thymine (T) will be uridine (U). In some embodiments, the mRNA comprises the nucleic acids according to FIG. 40 or 58, wherein thymine (T) will be 1-methyl-psuedouridine. In some embodiments, the mRNA is modified. In some embodiments, the modification is a modified nucleotide such as S-methyl-cytidine and/or 6-methyl-adenosine and/or modified uridine. In some embodiments, the mRNA comprises the nucleic acids according to FIG. 40 or 58, wherein the poly A tail is about 85 to about 200 nucleotides long. In some embodiments, the mRNA comprises the nucleic acids according to FIG. 40 or 58, wherein the poly A tail is about 85 to about 110 nucleotides long. In some embodiments, the mRNA comprises the nucleic acids according to FIG. 40 or 58, wherein the poly A tail is about 90 to about 110 nucleotides long. In some embodiments, the mRNA comprises the nucleic acids according to FIG. 40 or 58, wherein thymine (T) will be uridine (U) and wherein the sequence comprises the nucleotides up to the poly A tail, wherein the mRNA comprises a poly A tail about 85 to about 200 nucleotides long. In some embodiments, the mRNA comprises the nucleic acids according to FIG. 40 or 58, wherein thymine (T) will be uridine (U) and wherein the sequence comprises the nucleotides up to the poly A tail, wherein the mRNA comprises a poly A tail about 85 to about 110 nucleotides long. In some embodiments, the mRNA comprises the nucleic acids according to FIG. 40 or 58, wherein thymine (T) will be uridine (U) and wherein the sequence comprises the nucleotides up to the poly A tail, wherein the mRNA comprises a poly A tail about 90 to about 110 nucleotides long. In some embodiments, the mRNA comprises the nucleic acids according to FIG. 40 or 58, wherein thymine (T) will be 1-methyl-psuedouridine and wherein the sequence comprises the nucleotides up to the poly A tail, wherein the mRNA comprises a poly A tail about 85 to about 200 nucleotides long. In some embodiments, the mRNA comprises the nucleic acids according to FIG. 40 or 58, wherein thymine (T) will be 1-methyl-psuedouridine and wherein the sequence comprises the nucleotides up to the poly A tail, wherein the mRNA comprises a poly A tail about 85 to about 110 nucleotides long. In some embodiments, the mRNA comprises the nucleic acids according to FIG. 40 or 58, wherein thymine (T) will be 1-methyl-psuedouridine and wherein the sequence comprises the nucleotides up to the poly A tail, wherein the mRNA comprises a poly A tail about 90 to about 110 nucleotides long. In non-limiting embodiments, the mRNA is administered as an LNP.

In certain embodiments, the invention provides compositions comprising a nanoparticle which comprises any one of the envelopes of the invention, e.g., an envelope polypeptide as disclosed in Table 5 or a mRNA encoding an envelope polypeptide as disclosed in Table 6.

In certain embodiments, the invention provides compositions comprising a nanoparticle which comprises any one of the envelopes of the invention, e.g., an envelope polypeptide as disclosed in Table 5 or a mRNA encoding an envelope polypeptide as disclosed in Table 6, wherein the nanoparticle is a ferritin self-assembling nanoparticle.

In certain aspects, the invention provides a composition comprising a nanoparticle and a carrier, wherein the nanoparticle comprises trimers of any of the recombinant HIV-1 envelopes, e.g. an envelope polypeptide as disclosed in Table 5 or a mRNA encoding an envelope polypeptide as disclosed in Table 6. In certain embodiments, the nanoparticle is a ferritin self-assembling nanoparticle. In certain embodiments, the nanoparticle comprises multimers of trimers. Provided also are method for using these compositions comprising nanoparticles.

In certain embodiments, the invention provides a method of inducing an immune response in a subject comprising administering an immunogenic composition comprising any one of the recombinant HIV-1 envelopes of the invention e.g., an envelope polypeptide as disclosed in Tables 5 or a mRNA encoding an envelope polypeptide as disclosed in Table 6, or compositions comprising these recombinant HIV-1 envelopes, in an amount sufficient to induce an immune response. In certain embodiments, the composition is administered as a prime and/or a boost. In certain embodiments, the composition is administered as a single prime or as repetitive immunization prime. In preferred embodiments, the repetitive immunization is administered 3 or 4 times. In certain embodiments, the composition is administered as single boost or as a repetitive series of boosts. In preferred embodiments, the repetitive series of boosts is administered 3 or 4 times. In certain embodiments, the composition comprises nanoparticles. In certain embodiments, methods of the invention further comprise administering an adjuvant.

In certain embodiments, the subject is infected with HIV (e.g., HIV-1). In certain embodiments, the subject is an HIV-uninfected individual. In certain embodiments, the subject is an HIV-infected individual. In certain embodiments, the administration to the HIV-infected individual induces broadly neutralizing antibodies. In certain embodiments the broadly neutralizing antibodies of the HIV-infected individual mediates viral (e.g., HIV-1) clearance from blood and tissues.

In some embodiments, the composition is a first composition administered as a prime. In some embodiments, the composition is a second composition administered as one or more boosts. In some embodiments, the method comprises administering the first composition as a prime and administering the second composition as one or more boosts. In preferred embodiments, the first composition and the second composition are different.

In certain embodiments, the invention provides a composition comprising a plurality of nanoparticles comprising a plurality of the recombinant HIV-1 envelopes or trimers of the invention, e.g., an envelope polypeptide as disclosed in Tables 5 or a mRNA encoding an envelope polypeptide as disclosed in Table 6. In non-limiting embodiments, the envelopes/trimers of the invention are multimeric when comprised in a nanoparticle. The nanoparticle size is suitable for delivery. In non-liming embodiments the nanoparticles are ferritin based nanoparticles.

In certain aspects, the invention provides nucleic acids comprising sequences encoding proteins of the invention, e.g., encoding an envelope polypeptide as disclosed in Table 5 or comprising a mRNA encoding an envelope polypeptide as disclosed in Table 6. In certain embodiments, the nucleic acids are DNAs. In certain embodiments, the nucleic acids are mRNAs, modified or unmodified, suitable for use any use, e.g but not limited to use as pharmaceutical compositions. In certain aspects, the invention provides expression vectors comprising the nucleic acids of the invention. In some embodiments, the expression vector comprises DNA.

In certain aspects, the invention provides a pharmaceutical composition comprising nucleic acids (e.g., mRNAs) encoding the inventive HIV-1 envelopes, e.g., encoding an envelope polypeptide as disclosed in Table 5 or comprising a mRNA encoding an envelope polypeptide as disclosed in Table 6. In certain embodiments, these are optionally formulated in lipid nanoparticles (LNPs). In certain embodiments, the mRNAs are modified. Modifications include without limitations modified ribonucleotides, poly-A tail, 5′cap.

In certain embodiments, the nucleic acids are formulated in lipid, such as but not limited to LNPs. Non-limiting embodiments include LNPs without polyethylene glycol.

In certain aspects the invention provides nucleic acids encoding the inventive protein designs. In non-limiting embodiments, the nucleic acids are mRNA, modified or unmodified, suitable for any use, e.g but not limited to use as pharmaceutical compositions. In certain embodiments, the nucleic acids are formulated in lipid, such as but not limited to LNPs.

In certain aspects the invention provides a method of inducing an immune response comprising administering an immunogenic composition comprising a prime immunogen followed by at least one boost immunogen from Tables 5 or 6, wherein the boost immunogens are administered in an amount sufficient to induce an immune response. In certain embodiments, the prime is one of the CH848.0949.10.17DT, CH848.0949.10.17Dte, CH848.d0949.10.17DT.GS, or CH848.d0949.10.17DT.GS comprising additional modifications D230N.H289N.P291S.E169K designs. See Table 2 and WO2022/087031 which content is herein incorporated by reference in its entirety. In certain embodiments, the first boost is one of the CH848.0949.10.17WT, CH848.0949.10.17Wte designs. See Table 2 and WO2022/087031 which content is herein incorporated by reference in its entirety. In certain embodiments, the first boost is one of the CH848.0949.10.17DT or CH848.0949.10.17Dte designs. See Table 2. In certain embodiments, the boost is CH848.0358.80.06 or CH848.1432.5.41. In some embodiments, the modification to the V1 loop described herein can be incorporated into the envelope used as the prime and/or boost. In some embodiments, the method further comprises administering an immunogenic composition comprising any HIV-1 envelope sequence from the CH848 infected individual and variants thereof comprising the modification to the V1 loop described herein. In some embodiments, the method comprises administering an immunogenic composition comprising any HIV-1 envelope sequence from the CH848 infected individual and variants thereof comprising the modification to the V1 loop described herein as a prime.

In certain embodiments, the methods further comprise administering a boost from Table 4, wherein the boost is CH848.0808.15.15 in any suitable form.

In certain embodiments, the methods further comprise administering a boost from Table 4, wherein the boost is CH848.0358.80.06 in any suitable form.

In certain embodiments, the methods further comprise administering a boost from Table 4, wherein the boost is CH848.1432.5.41 in any suitable form.

In certain embodiments, the methods further comprise administering a boost from Table 4, wherein the boost is CH848.1621.4.44 in any suitable form.

In certain embodiments, the methods further comprise administering a boost from Table 4, wherein the boost is CH848.1305.10.35 in any suitable form.

In certain embodiments, the methods further comprise comprising administering a boost from Table 4, wherein the boost is P0402.c2.11 (G) in any suitable form.

In certain embodiments, the methods further comprise administering a boost from Table 4, wherein the boost is ZM246F (C) in any suitable form.

In certain embodiments, the methods further comprise administering a boost CH848.0358.80.06 in any suitable form.

In certain embodiments, the methods further comprise administering a boost CH848.1432.5.41 in any suitable form.

In certain embodiments, the methods further comprise administering a prime from Tables 5 or 6, wherein the prime is an envelope from Tables 5 or 6 in any suitable form. In certain embodiments, the prime comprises envelope CH848.0949.10.17DT comprising residues 132-154 of the N332-GT5 V1 loop.

In certain embodiments, the prime and/or boost immunogen are administered as a nanoparticle. In certain embodiments, the nanoparticle is a ferritin nanoparticle. In certain embodiments, the methods further comprise administering the prime and/or boost immunogen as a mRNA-LNP formulation.

In certain embodiments, the methods further comprise administering any suitable adjuvant.

### BRIEF DESCRIPTION OF DRAWINGS

The patent or application file contains at least one drawing executed in color. To conform to the requirements for PCT patent applications, many of the figures presented herein are black and white representations of images originally created in color.

FIG. 1 shows structure-based design of a single Env immunogen capable of binding to both DH270 UCA3 and BG18UCA.

FIG. 2 shows expression and purification of the V1 swap trimer.

FIG. 3 shows that V1swap mutations expand the UCA reactivity of the CH848 10.17DT Env.

FIG. 4 shows that V1swap binds to UCA from the DH270 and BG18 lineages with high apparent affinities.

FIG. 5 shows measuring mAb binding to cells transfected with V1swap gp160mRNA.

FIG. 6 shows the improvement of the V1swap trimer through mammalian cell display of a site-saturation library.

FIG. 7 shows the enrichment of the V1swap site saturation library for reactivity to both DH270 UCA3 and BG18 UCA.

FIG. 8 shows epitope presentation of the enriched V1swap site-saturation library.

FIGS. 9A-C show the design rationale for CH848 V1swap immunogen.

FIG. 10 shows that V3 glycan UCAs can be categorized based on their modes of Env engagement.

FIG. 11 shows the second generation V3-glycan germline targeting Env: V1 transplantation from N332-GT5 into the CH848 10.17DT background permits BG18 UCA reactivity.

FIGS. 12A-B shows that the V1swap mutations expand the UCA reactivity of the CH848 Env.

FIG. 13 shows the second generation V3-glycan germline targeting Envs: Translation to mRNA expressed gp160s

FIG. 14 shows the second generation V3-glycan germline targeting Env: Optimization of gp41 stability with proline mutations or GlySer linkers to boost expression.

FIG. 15 shows the second generation V3-glycan germline targeting Env: Optimization of gp41 stability by strengthening interprotomer interactions.

FIGS. 16-19 shows the evaluation of V1swap gp160 variants by high-throughput flow cytometry. The V1swap gp160s were expressed from mRNA constructs indicated.

FIGS. 20-31 show the heatmap and binding summary of all constructs with all antibodies listed in FIG. 20.

FIG. 32 shows that CH848_V1-swap Envelope gp160 reacts with V3-glycan UCAs.

FIG. 33 shows the downselection of mRNAs encoding gp160s.

FIG. 34 shows the third generation V3-glycan germline targeting Env.

FIG. 35 shows the third generation V3-glycan germline targeting Env: Display library is designed to vary V1-swap residues surrounding the DH270 UCA and BG18 UCA epitopes.

FIG. 36 shows the third generation V3-glycan germline targeting Env: Characterization of cell-surfaced expressed V1-swap envelope.

FIG. 37 shows the third generation V3-glycan germline targeting Env: Verification of expression of V1-swap envelope library.

FIG. 38 shows non-limiting embodiments of amino acid and nucleic acid (DNA) sequence of envelopes of the invention. Single peptide is underlined—the signal peptide is MPMGSLQPLATLYLLGMLVASVLA (SEQ ID NO: 3). HV1302795 is a variant of HV1302794 at the bold and italic amino acids. FIG. 38 discloses SEQ ID NOS 19-30, 33 and 35-51, respectively, in order of appearance.

FIG. 39 shows non-limiting embodiments of amino acid sequence of envelopes of the invention. Single peptide is underlined—the signal peptide is MPMGSLQPLATLYLLGMLVASVLA (SEQ ID NO: 3). FIG. 39 discloses SEQ ID NOS 52-63, respectively, in order of appearance.

FIG. 40 shows non-limiting embodiments of nucleic acid (e.g., for production as mRNA) sequence of envelopes. FIG. 40 discloses SEQ ID NOS 64-94, respectively, in order of appearance.

FIG. 41 shows non-limiting embodiments of nucleic acid sequences of envelopes. FIG. 41 discloses SEQ ID NOS 95-102, respectively, in order of appearance.

FIG. 42 shows non-limiting embodiments of amino acid sequences of envelopes. FIG. 42 discloses SEQ ID NOS 103-110, respectively, in order of appearance.

FIG. 43 shows non-limiting embodiments of the sortase design of an envelope. FIG. 43 discloses SEQ ID NOS 111-114, respectively, in order of appearance.

FIGS. 44A-B show non-limiting embodiments for sequences comprising amino acid Arg327 (K327R). In the amino acid sequences (FIG. 44B), underlined is the signal peptide and the preceding four amino acids indicate the cloning site/kozak sequence (VDTA (SEQ ID NO: 4)) neither of which that would not be part of the final recombinant protein. FIG. 44A-B discloses SEQ ID NOS 115-128, respectively, in order of appearance.

FIGS. 45A-B show non-limiting embodiments of sequences comprising varying linkers between the envelope and ferritin proteins. In the amino acid sequences (FIG. 45B), underlined is the signal peptide and the preceding four amino acids indicate the cloning site/kozak sequence (VDTA (SEQ ID NO: 4)) neither of which that would not be part of the final recombinant protein. FIG. 45A-B discloses SEQ ID NOS 129-146, respectively, in order of appearance.

FIGS. 46A-B show non-limited embodiments of designs of 19CV3 sequences. Amino acids H66A_A582T_L587A are referred to JS2 or “joe2” mutations. In the amino acid sequences (FIG. 46B), underlined is the signal peptide and the preceding four amino acids indicate the cloning site/kozak sequence (VDTA (SEQ ID NO: 4)) neither of which that would not be part of the final recombinant protein. FIG. 46A-B discloses SEQ ID NOS 147-166, respectively, in order of appearance.

FIG. 47 shows one embodiment of a design for the production of trimeric HIV-1 Env on ferritin nanoparticles. FIG. 47 discloses “GGGGGG” as SEQ ID NO: 167 and “LPSTG” as SEQ ID NO: 168.

FIG. 48 shows non-limiting examples of envelopes designs and sequences described in Table 3. FIG. 48 discloses SEQ ID NOS 169-176, respectively, in order of appearance.

FIG. 49 shows non-limiting examples of envelope designs and sequences described in Table 4—envelopes CH848.0808.15.15, CH848.1621.4.44, CH848.1305.10.35, P0402.c2.11 (G), ZM246F (C). FIG. 49 discloses SEQ ID NOS 177-238, respectively, in order of appearance.

FIGS. 50A-B show non-limiting examples of designs and sequences. FIG. 50A shows non-limiting examples of designs and sequences based on envelope CH848.0358.80.06 and CH848.1432.5.41. FIG. 50B shows non-limiting examples of envelopes designs and sequences described in Table 4. FIG. 50A-B discloses SEQ ID NOS 239-261, respectively, in order of appearance.

FIG. 51 shows non-limiting examples of envelope designs and sequences of 10.17 DT.GS envelope designs. FIG. 51 discloses SEQ ID NOS 262-287, respectively, in order of appearance.

FIG. 52 shows non-limiting examples of V1swap envelope designs and sequences. FIG. 80 discloses SEQ ID NOs 288-303, respectively, in order of appearance.

FIG. 53 part 1 shows the CH848 V1swap new designs.

FIG. 53 part 2 shows the tested antibodies.

FIG. 53 part 3 shows the heatmap of the additional CH848 V1swap designs. The gp160s were expressed from mRNA constructs.

FIG. 53 parts 4-13 show MFI of the additional CH848 V1swap designs. The gp160s were expressed from mRNA constructs.

FIG. 53 parts 14-19 show the flow cytometry data of the additional CH848 V1swap designs. The gp160s were expressed from mRNA constructs.

FIG. 54 part 1 shows V1swap E169K S148G Q328M membrane-bound Env designs.

FIG. 54 part 2 shows the tested antibodies.

FIG. 54 part 3 shows the gating data of V1swap E169K S148G Q328M membrane-bound Env designs. The gp 160s were expressed from mRNA constructs.

FIG. 54 parts 4-7 show MFI of V1swap E169K S148G Q328M membrane-bound Env designs. The gp 160s were expressed from mRNA constructs.

FIG. 54 parts 8-9 show MFI normalized to N6 of V1swap E169K S148G Q328M membrane-bound Env designs. The gp160s were expressed from mRNA constructs.

FIG. 54 parts 10-12 show the flow cytometry data of V1swap E169K S148G Q328M membrane-bound Env designs. The gp160s were expressed from mRNA constructs. FIG. 55 part 1 shows V1swap with V3 stabilized designs.

FIG. 55 part 2 shows the tested antibodies.

FIG. 55 part 3 shows the heatmap of MFI of all antibodies binding to V1swap with V3 stabilized designs. The gp160s were expressed from mRNA constructs.

FIG. 55 parts 4-9 show MFI of V1swap with V3 stabilized designs. The gp160s were expressed from mRNA constructs.

FIG. 55 part 10 shows the 19b binding data normalized to N6.

FIG. 55 part 11 shows the tail truncation effects on the expression levels.

FIG. 56 part 1 shows the MB Env V1swap designs. The gp160s were expressed from mRNA constructs.

FIG. 56 part 2 shows the tested antibodies.

FIG. 56 part 3 shows the heatmap of MFI of all antibodies binding to MB Env V1swap designs. The gp 160s were expressed from mRNA constructs.

FIG. 56 parts 4-9 show MFI of MB Env V1swap designs. The gp160s were expressed from mRNA constructs.

FIG. 57 parts 1-2 show MFI of CH848 MD64 gp160 designs (combination of three sets of data). The gp 160s were expressed from mRNA constructs.

FIG. 57 parts 3-4 show the flow cytometry of CH848 MD64 gp160 designs (first set). The gp160s were expressed from mRNA constructs.

FIG. 57 parts 5-7 show MFI of CH848 MD64 gp 160 designs (third set). The gp160s were expressed from mRNA constructs.

FIG. 57 parts 8-11 show flow cytometry of CH848 MD64 gp160 designs (third set). The gp160s were expressed from mRNA constructs.

FIG. 57 parts 12-14 show MFI of CH848 MD64 gp160 designs (second set). The gp160s were expressed from mRNA constructs.

FIG. 57 part 15 shows the heatmap of CH848 MD64 gp 160 designs (first and second sets). The gp160s were expressed from mRNA constructs.

FIG. 57 part 16 shows the MFI of the first and third sets of CH848 MD64 gp 160 design (first and second sets). The gp160s were expressed from mRNA constructs.

FIG. 57 part 17 shows the flow cytometry data of CH848 MD64 gp160 design (second set). The gp160s were expressed from mRNA constructs.

FIG. 57 parts 18-23 show MFI of CH848 MD64 gp160 design (first and second sets). The gp160s were expressed from mRNA constructs.

FIG. 57 parts 24-26 show MFI of CH848 MD64 gp160 design (first set). The gp 160s were expressed from mRNA constructs.

FIG. 57 part 27 shows the heatmap of the CH848 MD64 gp160 design (first set). The gp160s were expressed from mRNA constructs.

FIG. 57 part 28 shows MFI of the mock construct.

FIG. 57 part 29 shows the flow cytometry of the mock construct.

FIG. 57 part 30 shows MFI of CH848 MD64 gp160 design (first set). The gp160s were expressed from mRNA constructs.

FIG. 57 part 31 shows the flow cytometry of CH848 MD64 gp 160 design (first set). The gp160s were expressed from mRNA constructs.

FIG. 57 parts 32-35 show MFI of CH848 MD64 gp160 design (first set). The gp 160s were expressed from mRNA constructs.

FIG. 57 part 36 shows the flow cytometry of CH848 MD64 gp160 design (first set). The gp160s were expressed from mRNA constructs.

FIG. 57 parts 37-41 show MFI of CH848 MD64 gp160 design (first set). The gp 160s were expressed from mRNA constructs.

FIG. 58 shows non-limiting embodiments of nucleic acid (e.g., for production as mRNA) sequence of envelopes. FIG. 58 discloses SEQ ID NOS 304-330, respectively, in order of appearance

### DETAILED DESCRIPTION

The development of a safe, highly efficacious prophylactic HIV-1 vaccine is of paramount importance for the control and prevention of HIV-1 infection. A major goal of HIV-1 vaccine development is the induction of broadly neutralizing antibodies (bnAbs) (Immunol. Rev. 254:225-244, 2013). BnAbs are protective in rhesus macaques against SHIV challenge, but as yet, are not reproducibly induced by current vaccines.

For the past 25 years, the HIV vaccine development field has used single or prime boost heterologous Envs as immunogens, but to date has not found a regimen to induce high levels of bnAbs.

Recently, a new paradigm for design of strategies for induction of broadly neutralizing antibodies was introduced, that of B cell lineage immunogen design (Nature Biotech. 30:423, 2012) in which the induction of bnAb lineages is recreated. It was recently demonstrated the power of mapping the co-evolution of bnAbs and founder virus for elucidating the Env evolution pathways that lead to bnAb induction (Nature 496:469, 2013). The invention provides methods of using these pan bnAb envelope immunogens.

In certain aspects, the invention provides compositions for immunizations to induce lineages of broad neutralizing antibodies. In certain embodiments, there is some variance in the immunization regimen; in some embodiments, the selection of HIV-1 envelopes may be grouped in various combinations of primes and boosts, either as nucleic acids, proteins, or combinations thereof. In certain embodiments the compositions are pharmaceutical compositions which are immunogenic. In certain embodiments, the compositions comprise amounts of envelopes which are therapeutic and/or immunogenic.

In one aspect, the invention provides a composition for a prime boost immunization regimen comprising any one of the envelopes described herein, or any combination thereof wherein the envelope is a prime or boost immunogen. In certain embodiments the composition for a prime boost immunization regimen comprises one or more envelopes described herein.

In certain embodiments, the compositions contemplate nucleic acid, as DNA and/or RNA, or recombinant protein immunogens either alone or in any combination. In certain embodiments, the methods contemplate genetic, as DNA and/or RNA, immunization either alone or in combination with recombinant envelope protein(s).

In some embodiments the antigens are nucleic acids, including but not limited to mRNAs which could be modified and/or unmodified. See US Pub 20180028645A1, US Pub 20170369532, US Pub 20090286852, US Pub 20130111615, US Pub 20130197068, US Pub 20130261172, US Pub 20150038558, US Pub 20160032316, US Pub 20170043037, US Pub 20170327842, each content is incorporated by reference in its entirety. mRNAs delivered in LNP formulations have advantages over non-LNPs formulations. See US Pub 20180028645A1.

In certain embodiments the nucleic acid encoding an envelope is operably linked to a promoter inserted an expression vector. In certain aspects the compositions comprise a suitable carrier. In certain aspects the compositions comprise a suitable adjuvant.

In certain embodiments the induced immune response includes induction of antibodies, including but not limited to autologous and/or cross-reactive (broadly) neutralizing antibodies against HIV-1 envelope. Various assays that analyze whether an immunogenic composition induces an immune response, and the type of antibodies induced are known in the art and are also described herein.

In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects the invention provides nucleic acids comprising any one of the nucleic acid sequences of invention. In certain aspects the invention provides nucleic acids consisting essentially of any one of the nucleic acid sequences of invention. In certain aspects the invention provides nucleic acids consisting of any one of the nucleic acid sequences of invention. In certain embodiments the nucleic acid of the invention, is operably linked to a promoter and is inserted in an expression vector. In certain aspects the invention provides an immunogenic composition comprising the expression vector. In some embodiments, the expression vector comprises DNA.

In certain aspects the invention provides a composition comprising at least one of the nucleic acid sequences of the invention. In certain aspects the invention provides a composition comprising any one of the nucleic acid sequences of invention. In certain aspects the invention provides a composition comprising at least one nucleic acid sequence encoding any one of the polypeptides of the invention.

The envelope used in the compositions and methods of the invention can be a gp160, gp150, gp145, gp140, gp120, gp41, N-terminal deletion variants as described herein, cleavage resistant variants as described herein, or codon optimized sequences thereof. In certain embodiments the composition comprises envelopes as trimers. In certain embodiments, envelope proteins are multimerized, for example trimers are attached to a particle such that multiple copies of the trimer are attached and the multimerized envelope is prepared and formulated for immunization in a human. In certain embodiments, the compositions comprise envelopes, including but not limited to trimers as a particulate, high-density array on liposomes or other particles, for example but not limited to nanoparticles. In some embodiments, the trimers are in a well ordered, near native like or closed conformation. In some embodiments the trimer compositions comprise a homogenous mix of native like trimers. In some embodiments the trimer compositions comprise at least 85%, 90%, 95% native like trimers.

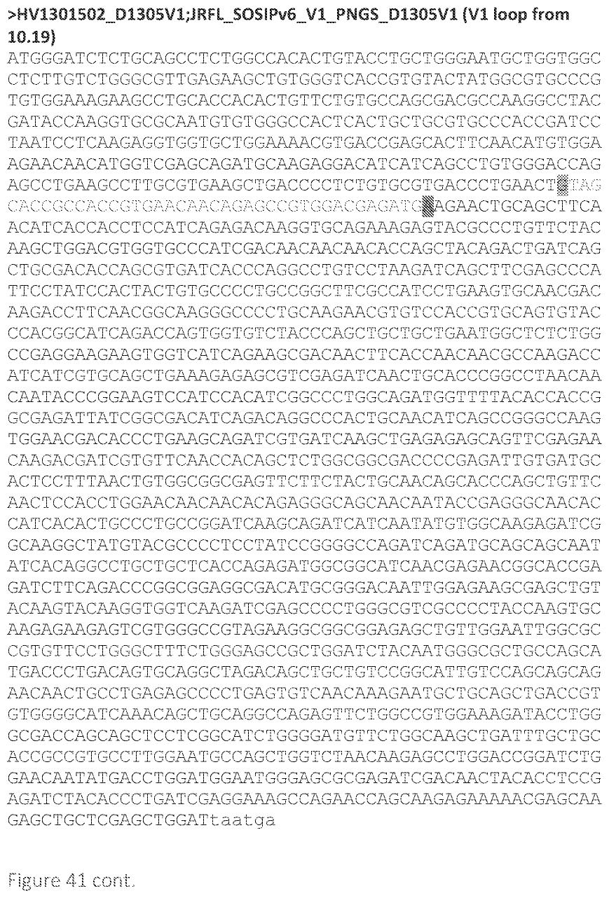

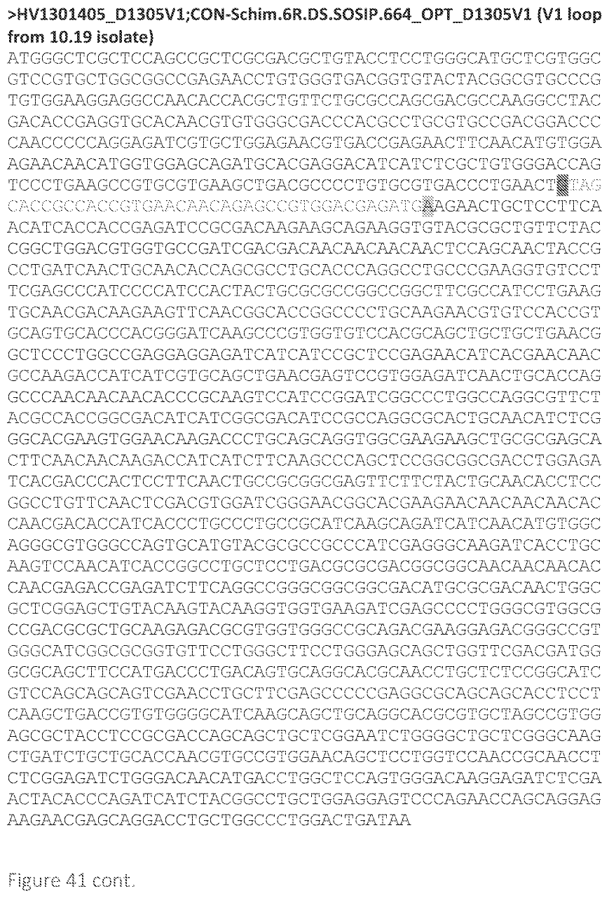

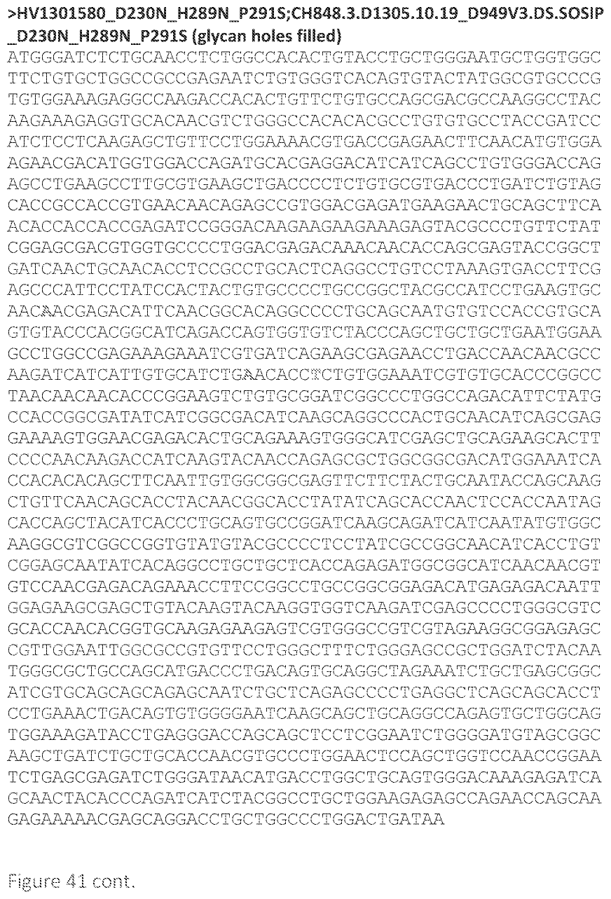

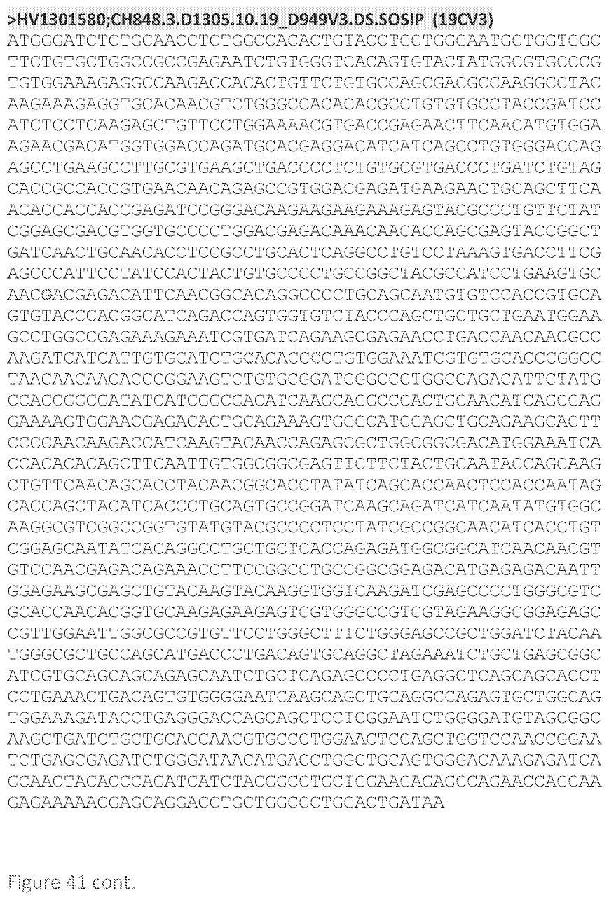

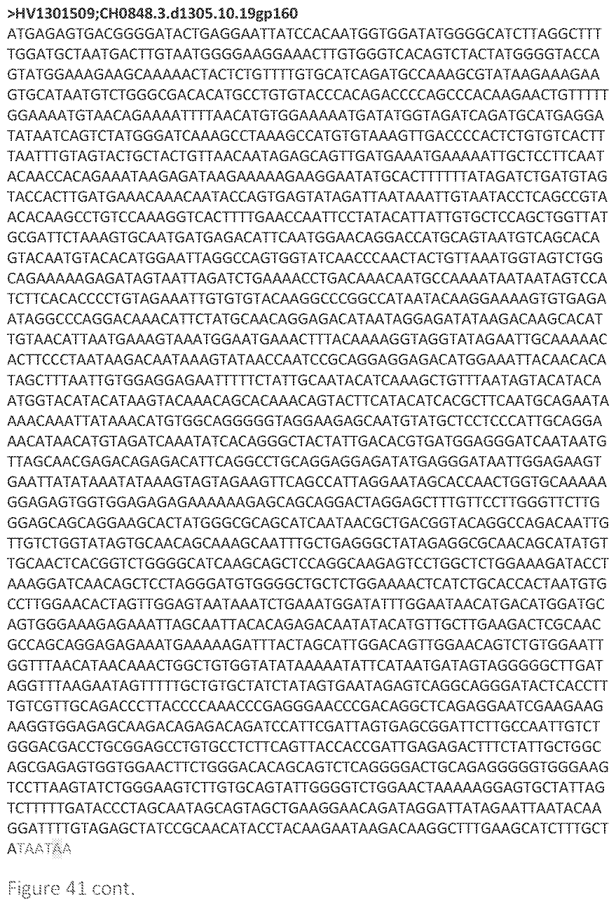

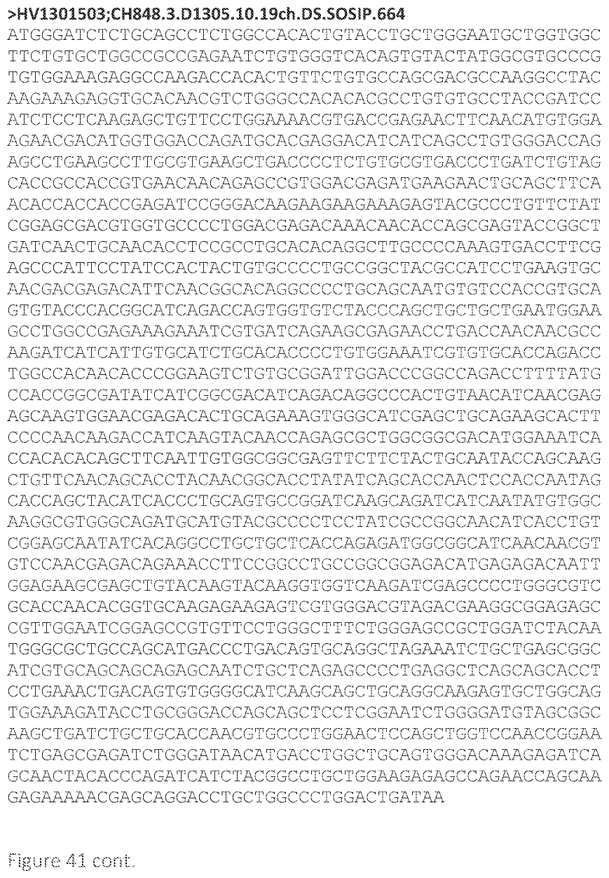

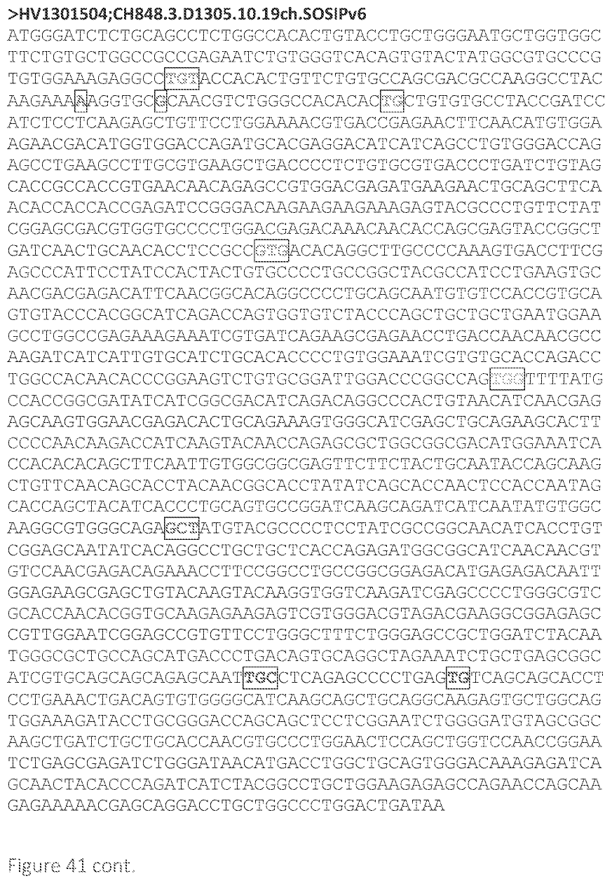

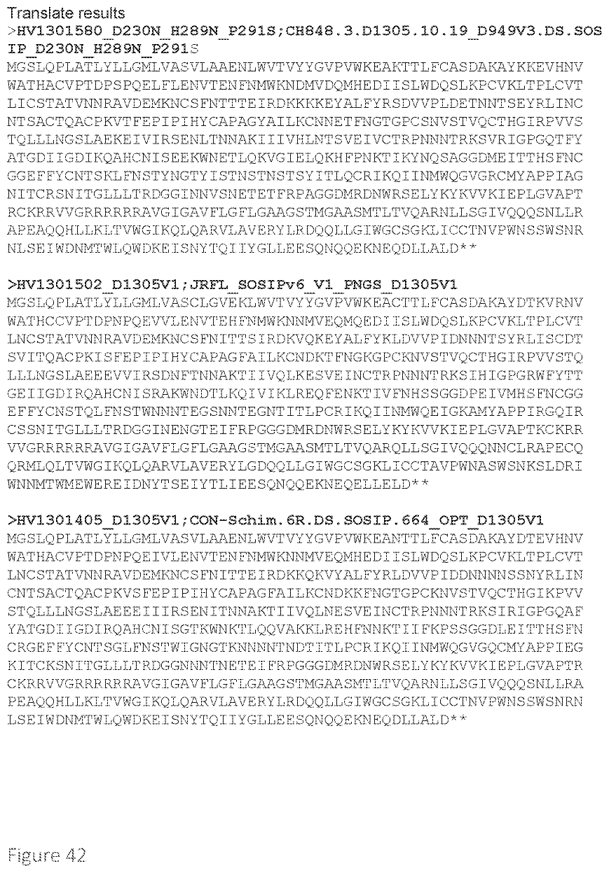

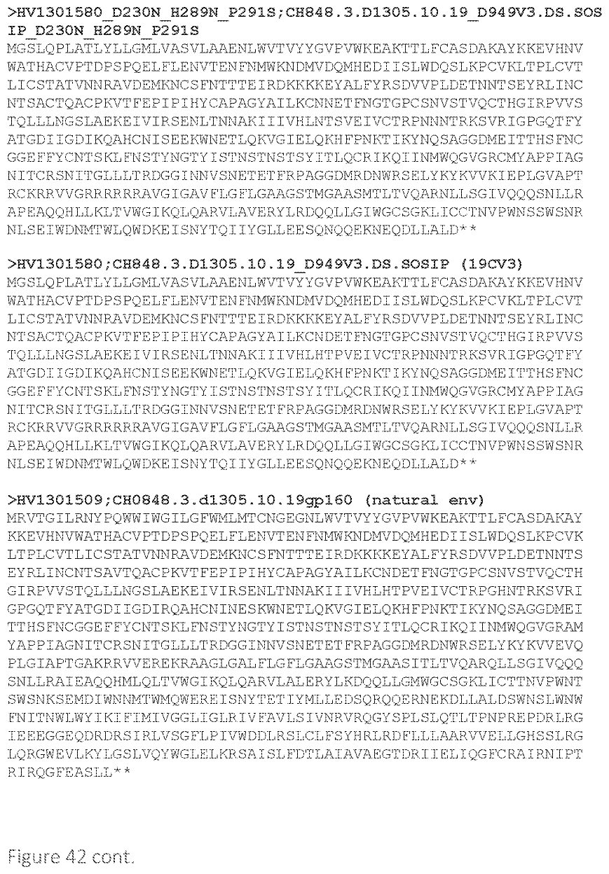

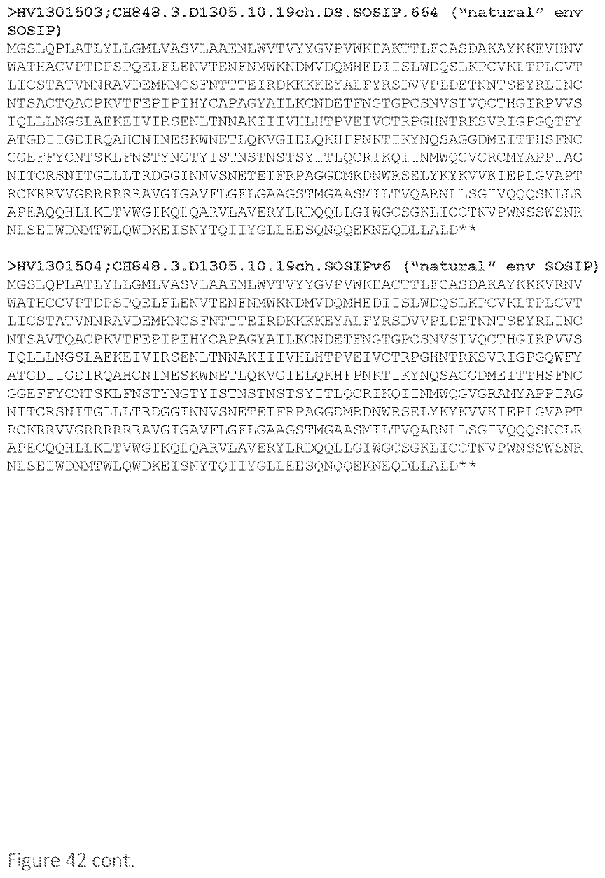

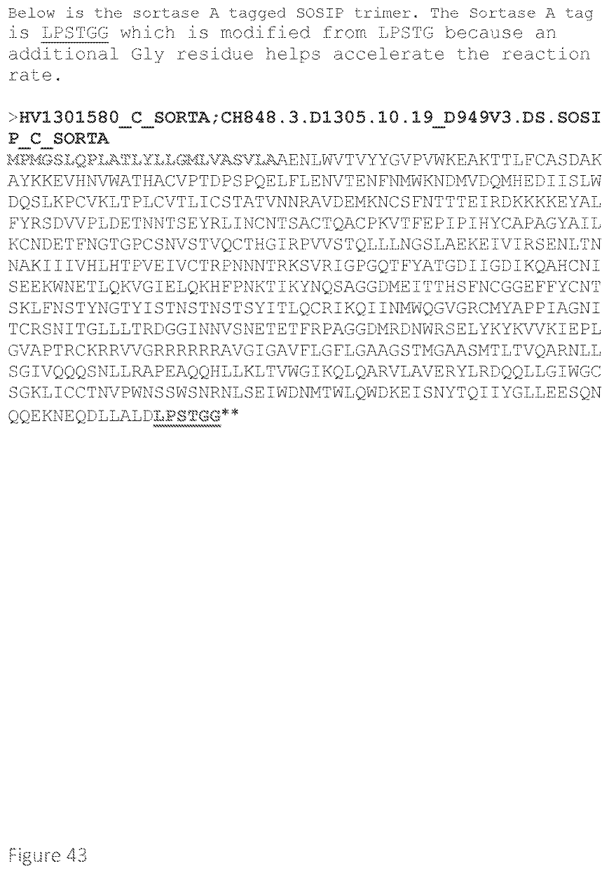

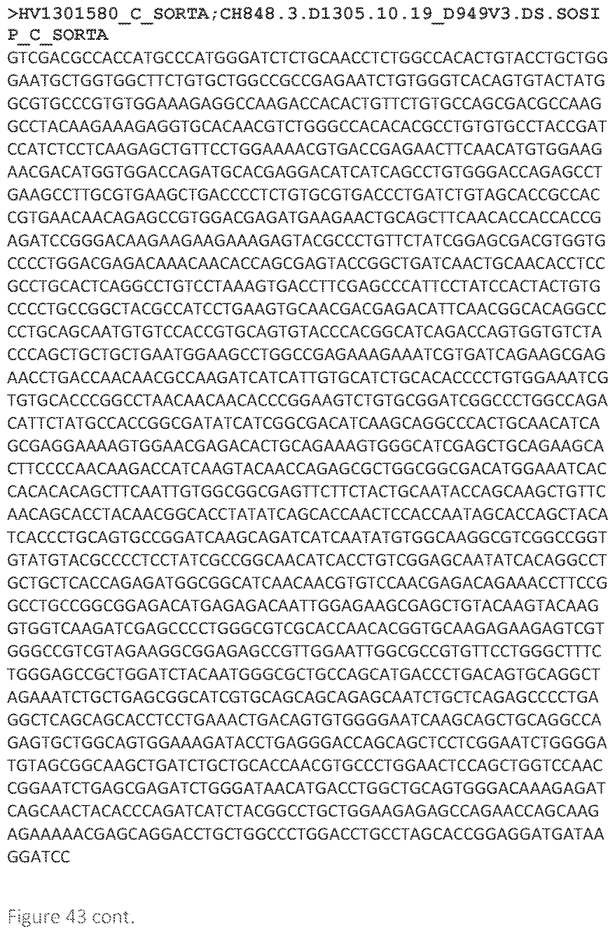

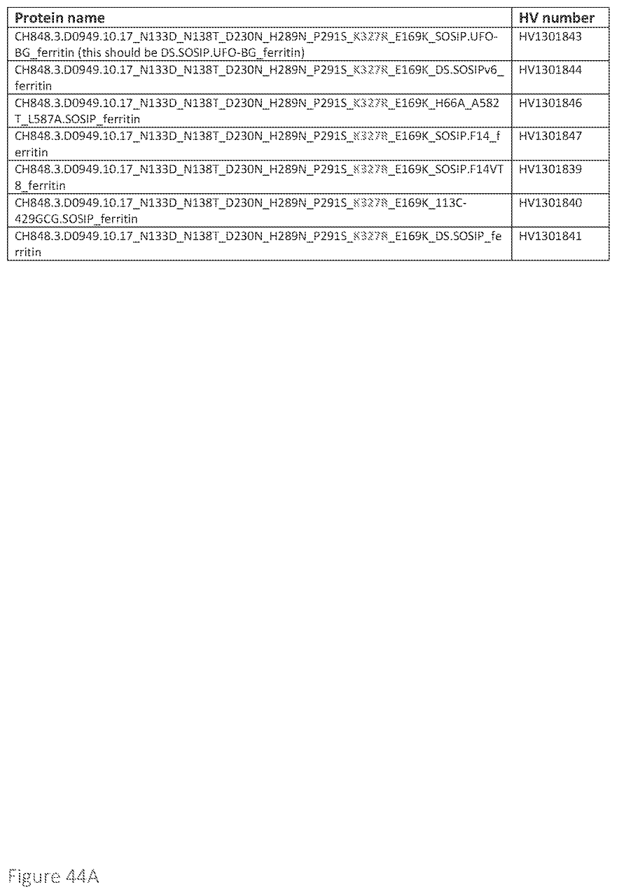

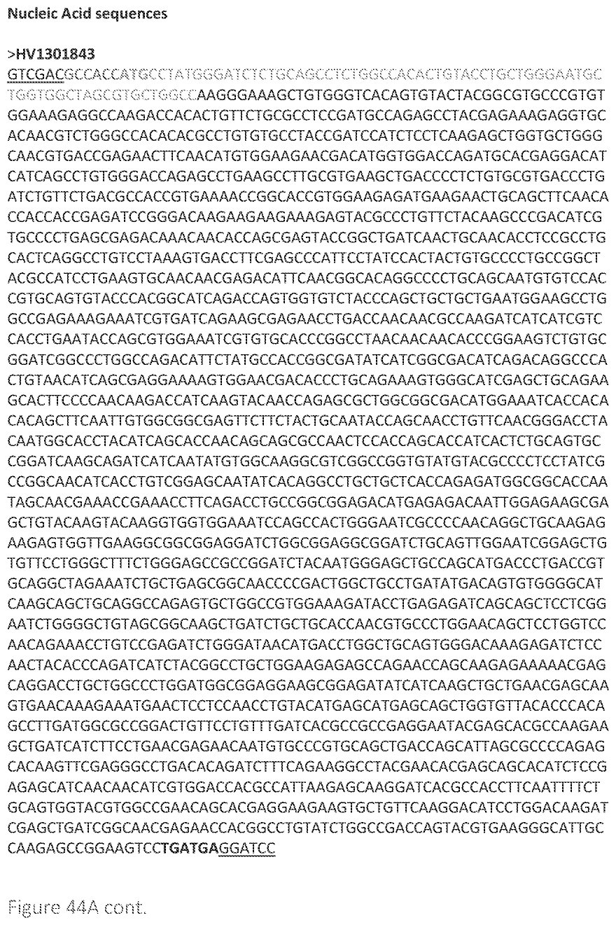

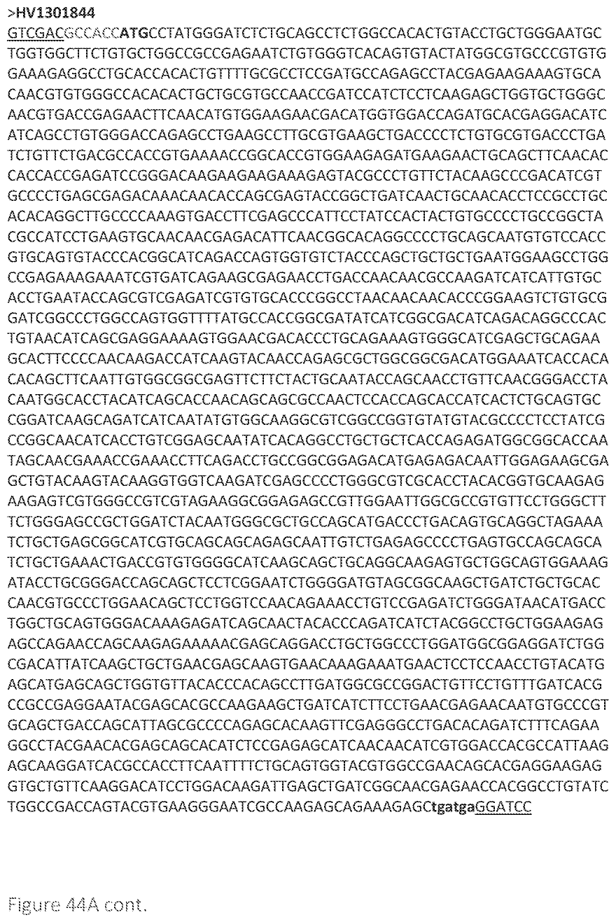

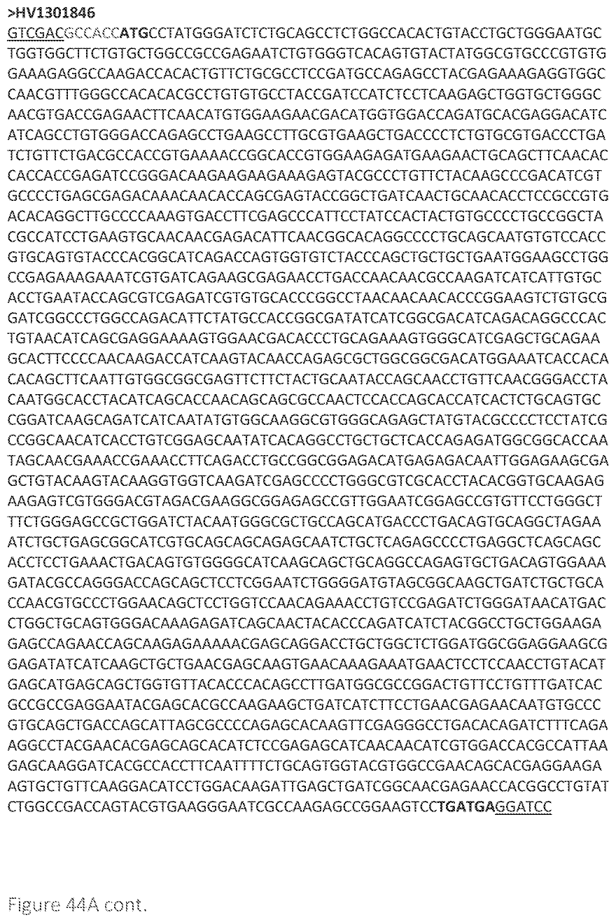

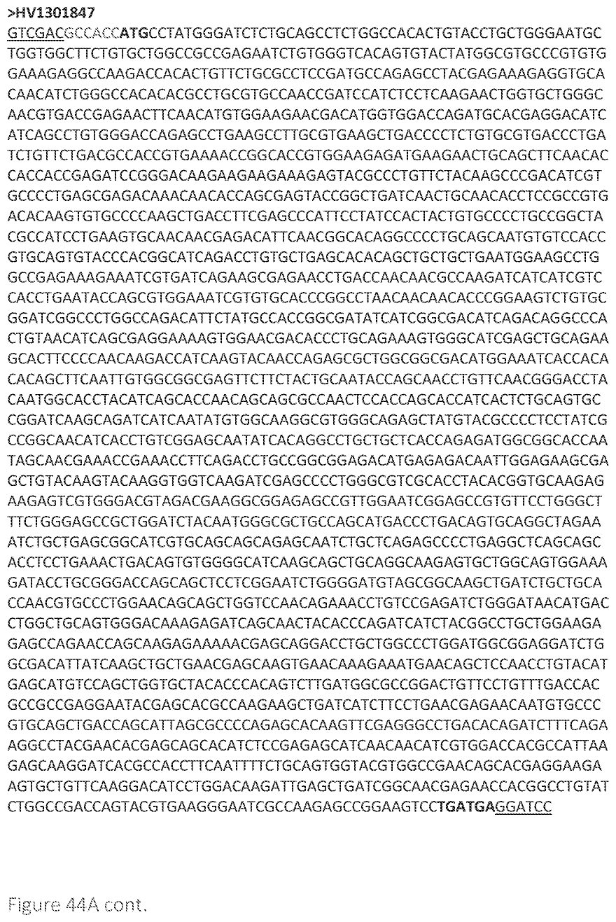

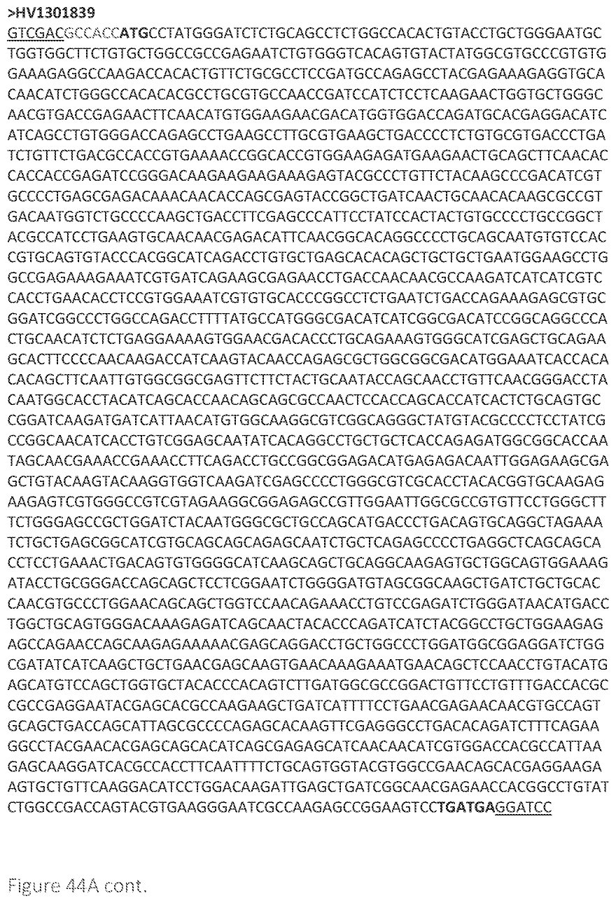

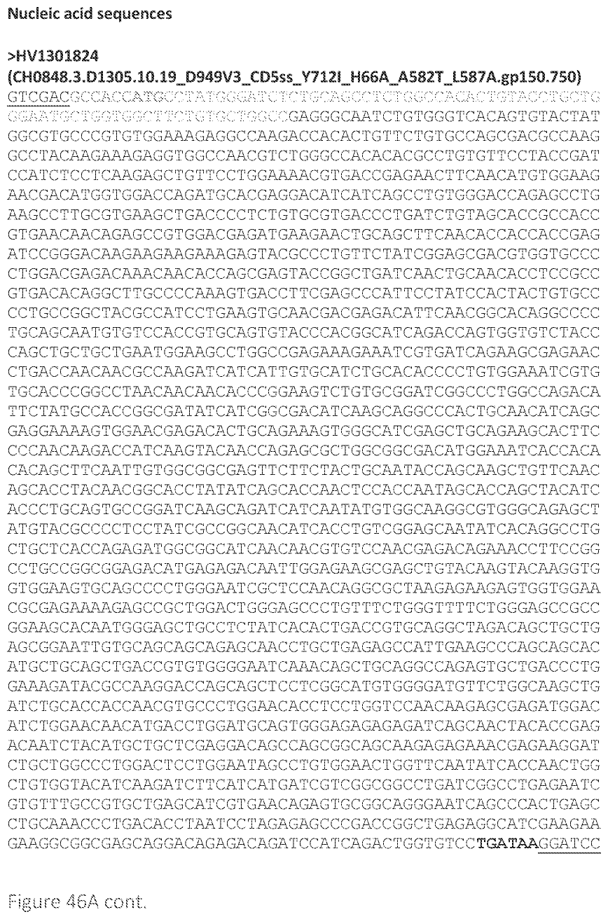

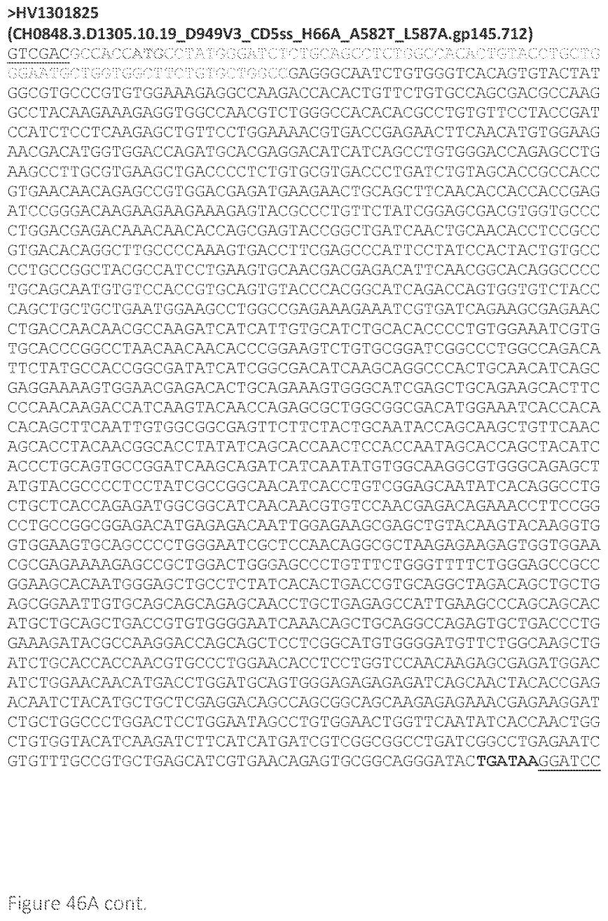

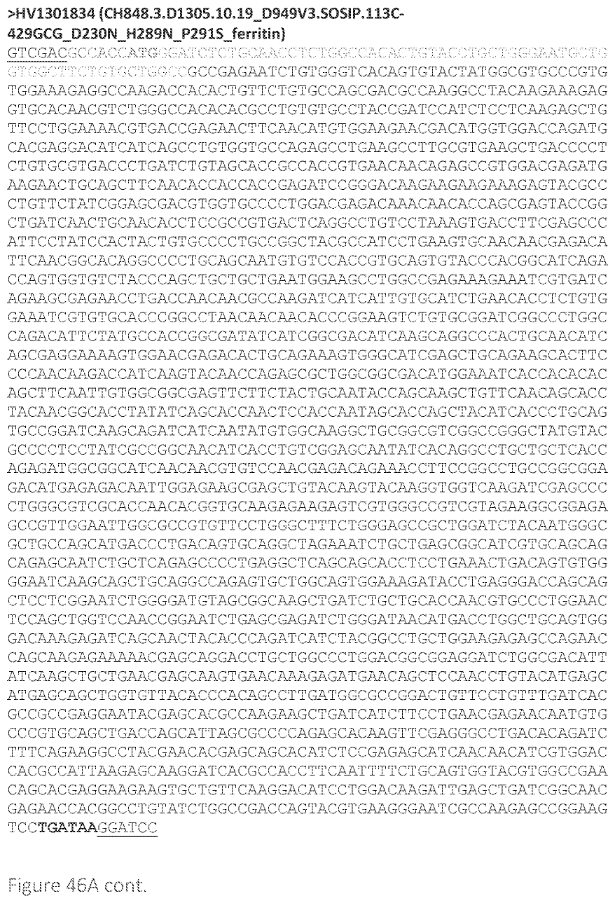

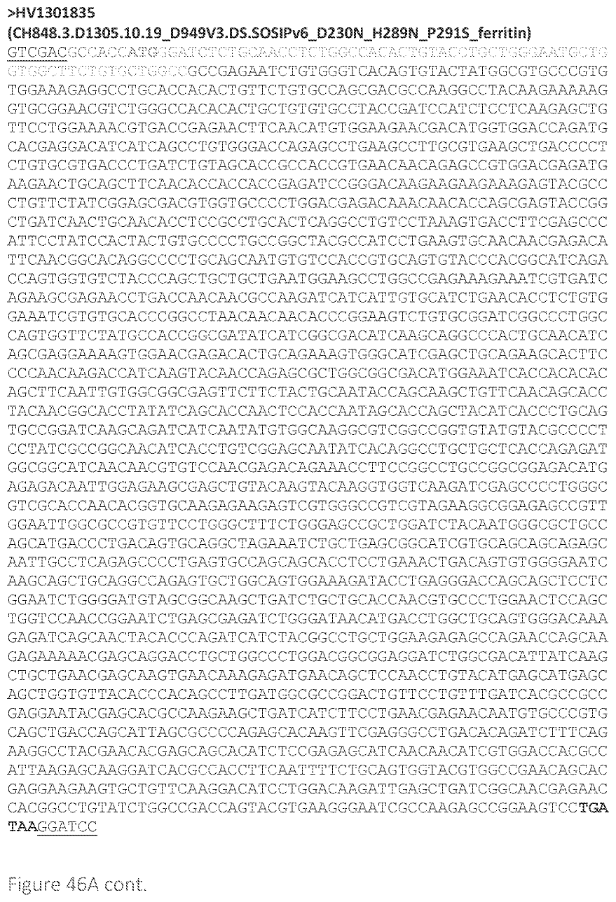

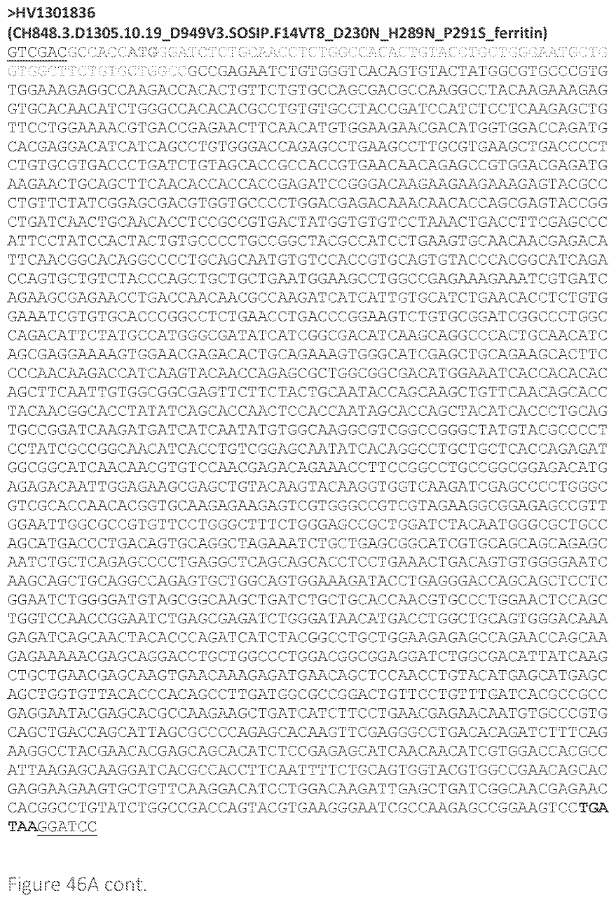

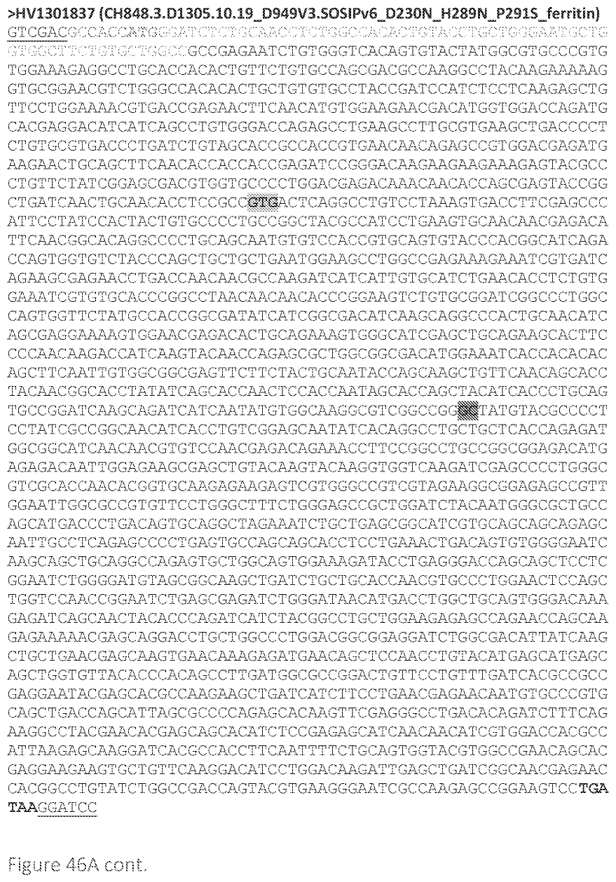

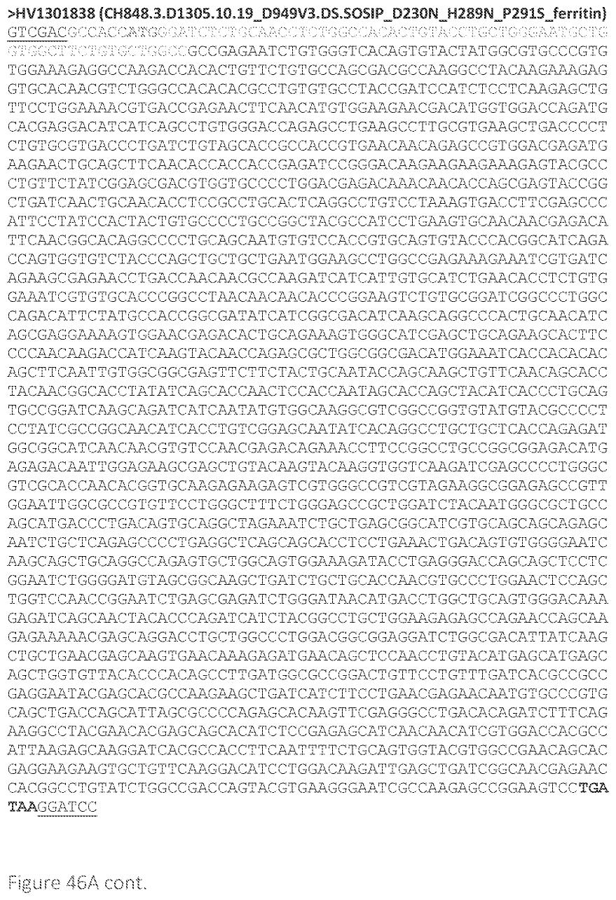

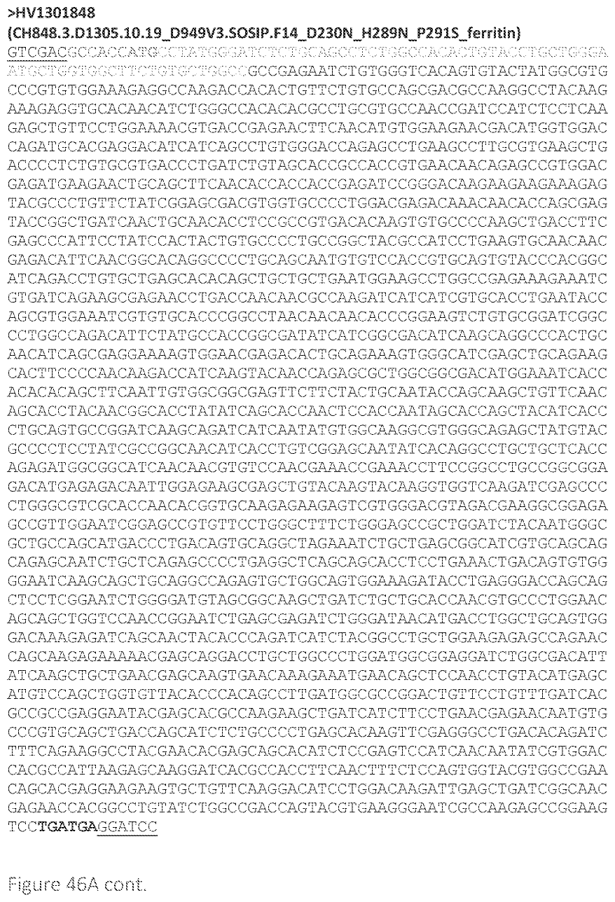

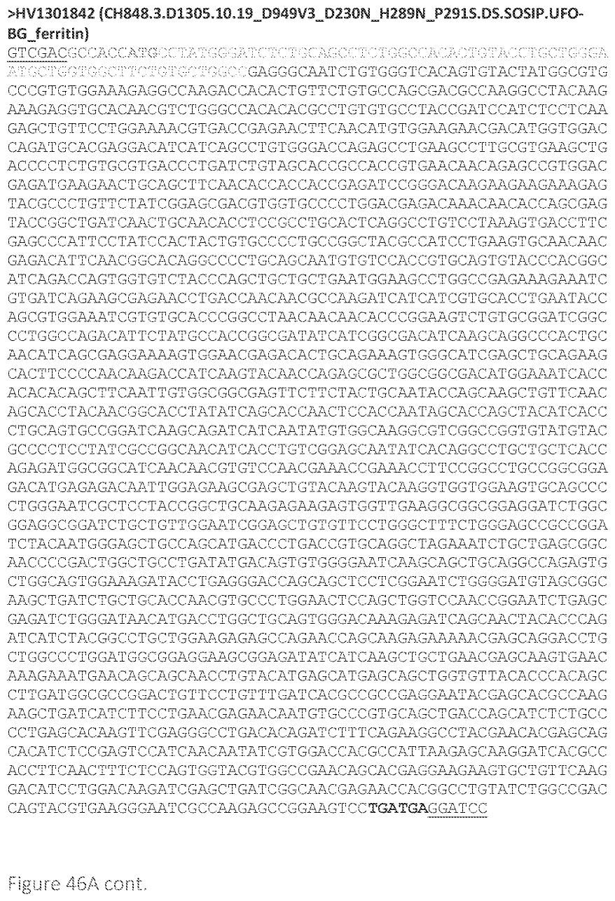

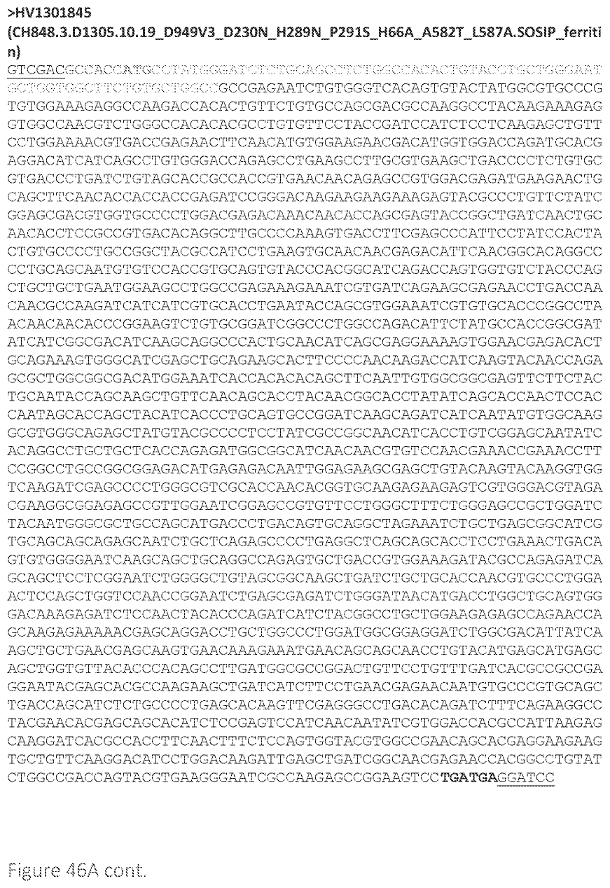

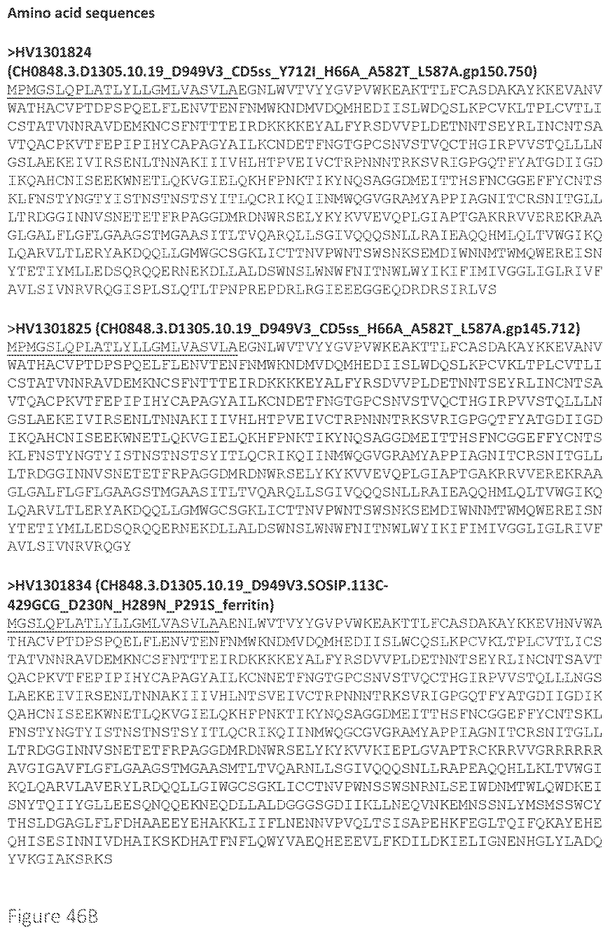

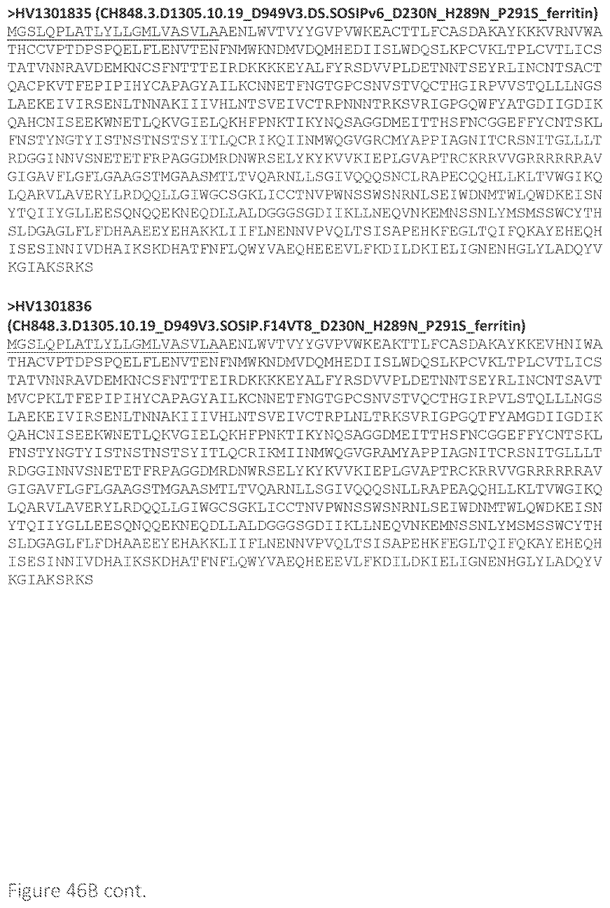

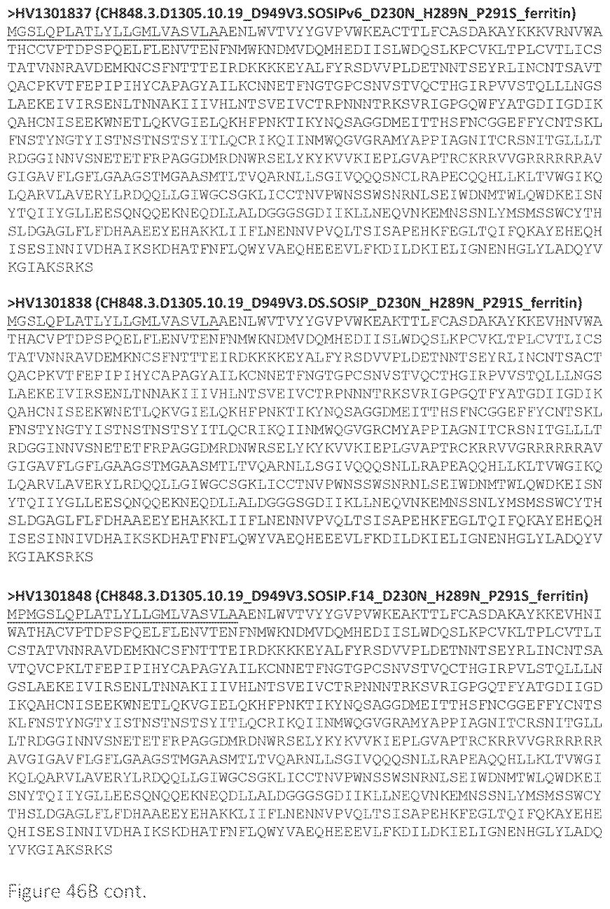

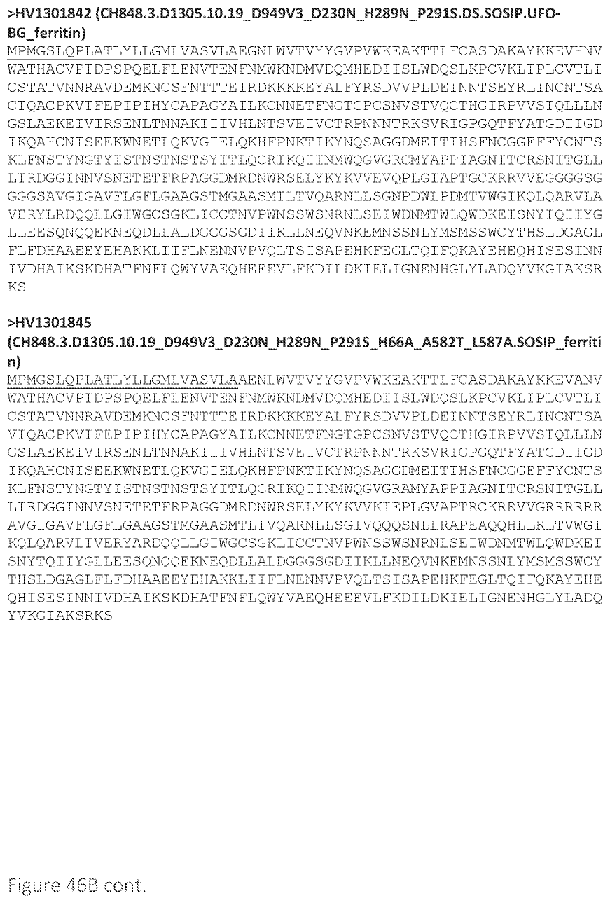

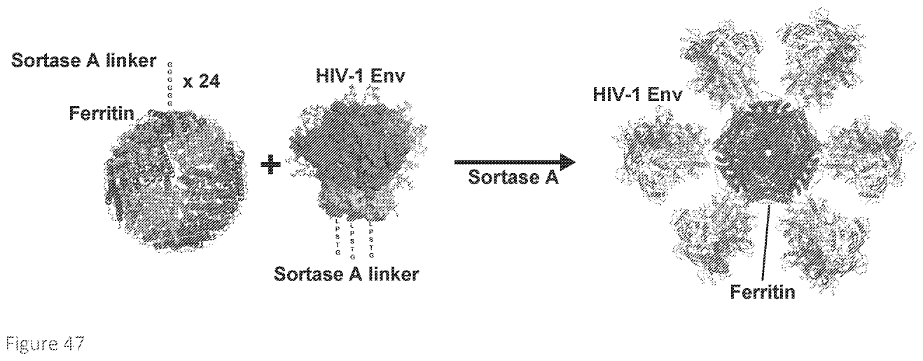

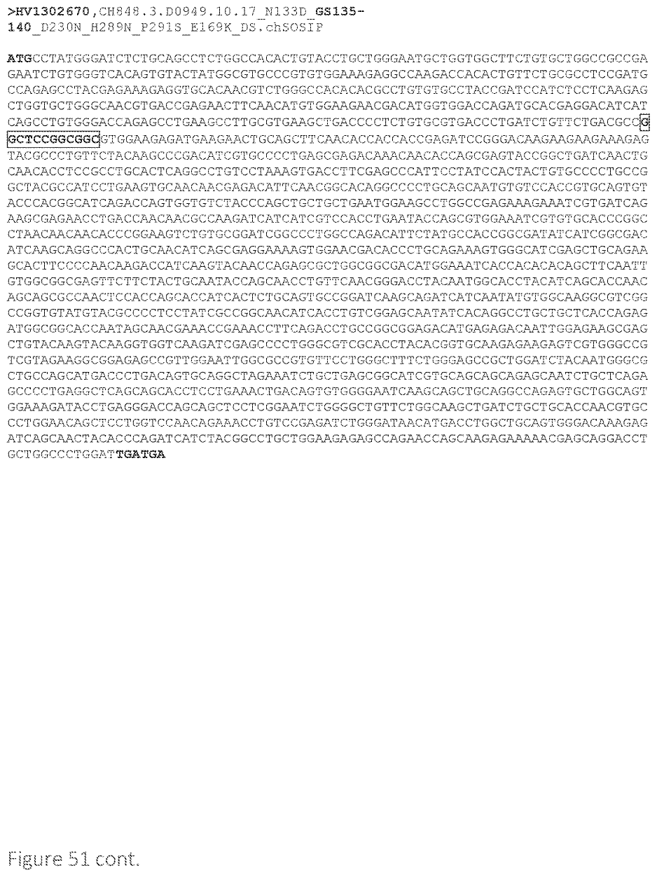

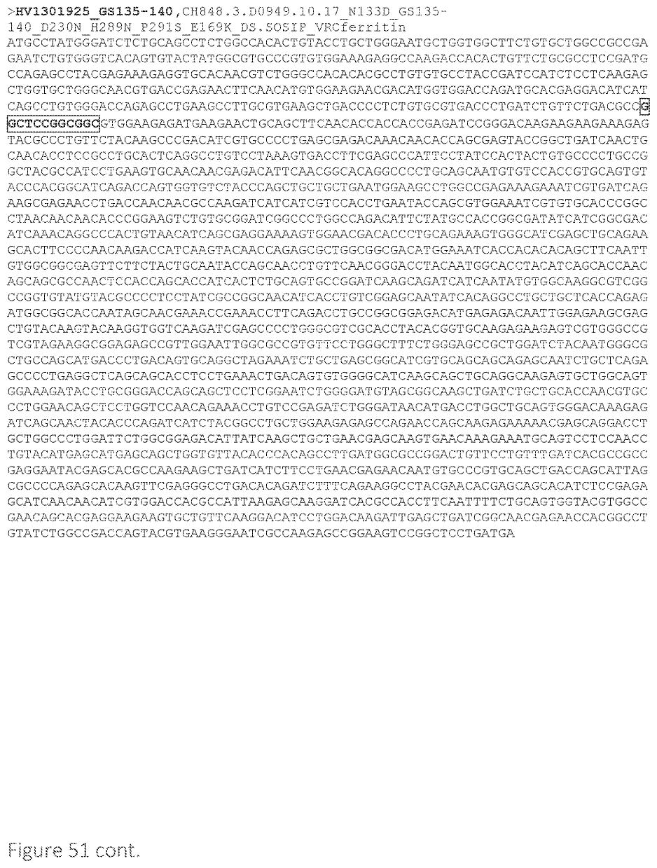

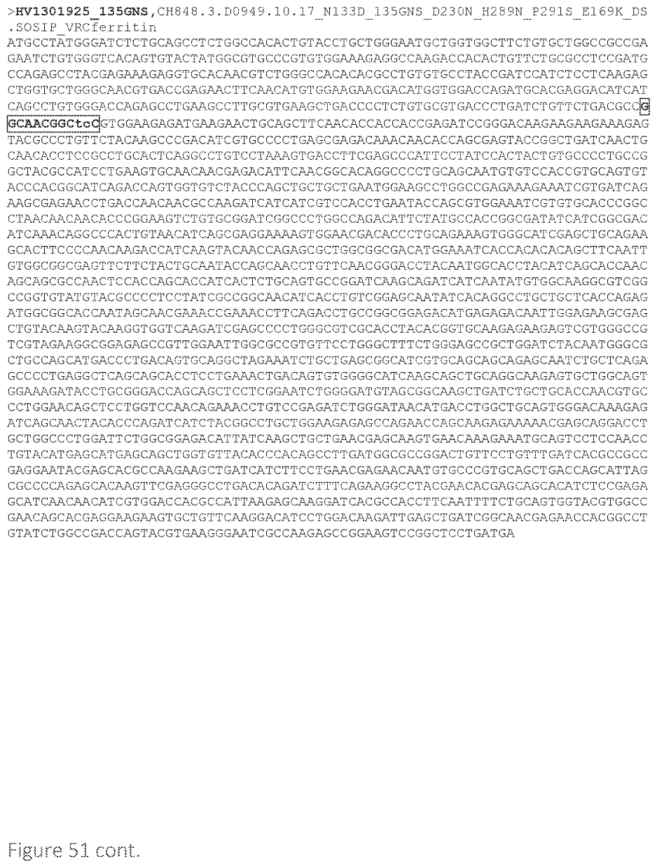

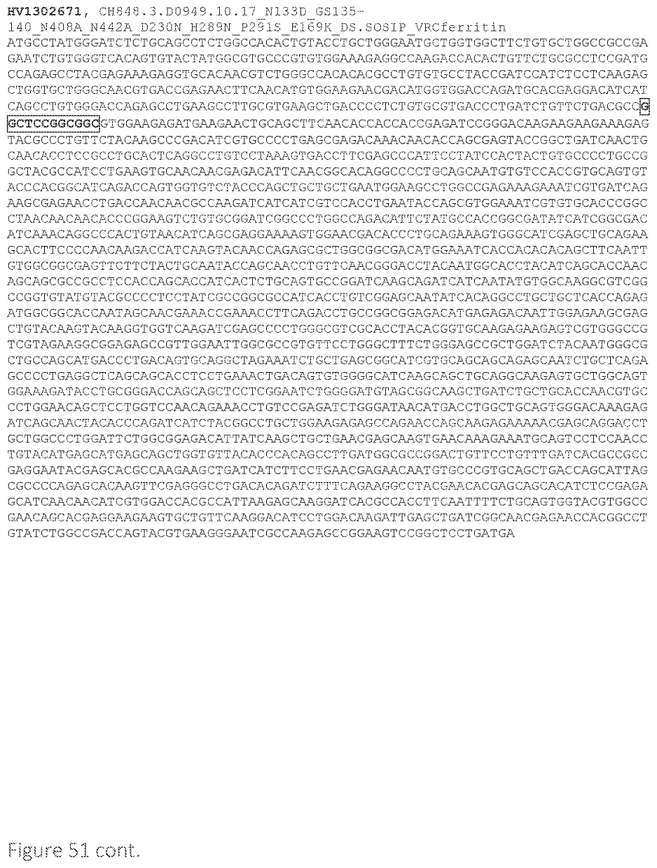

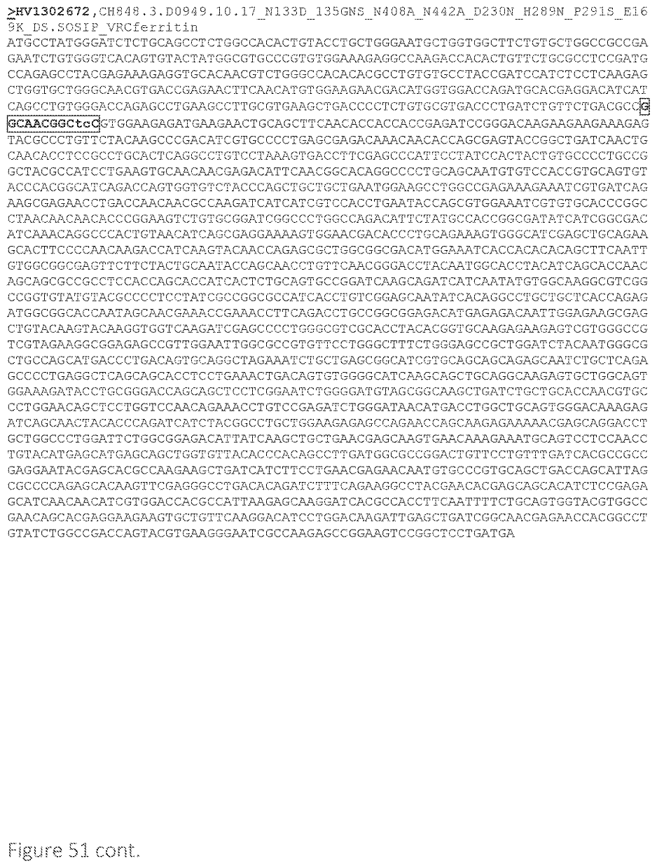

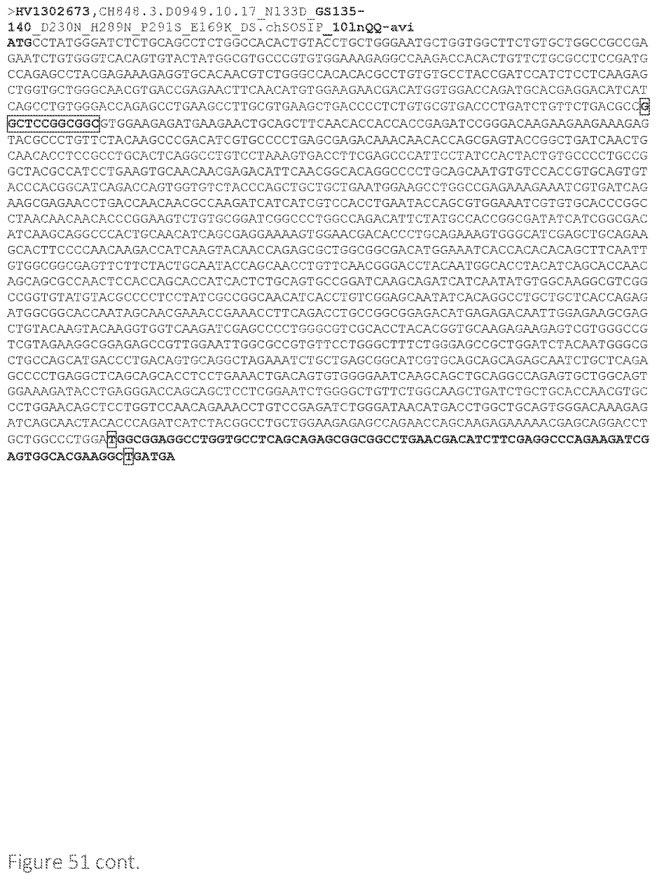

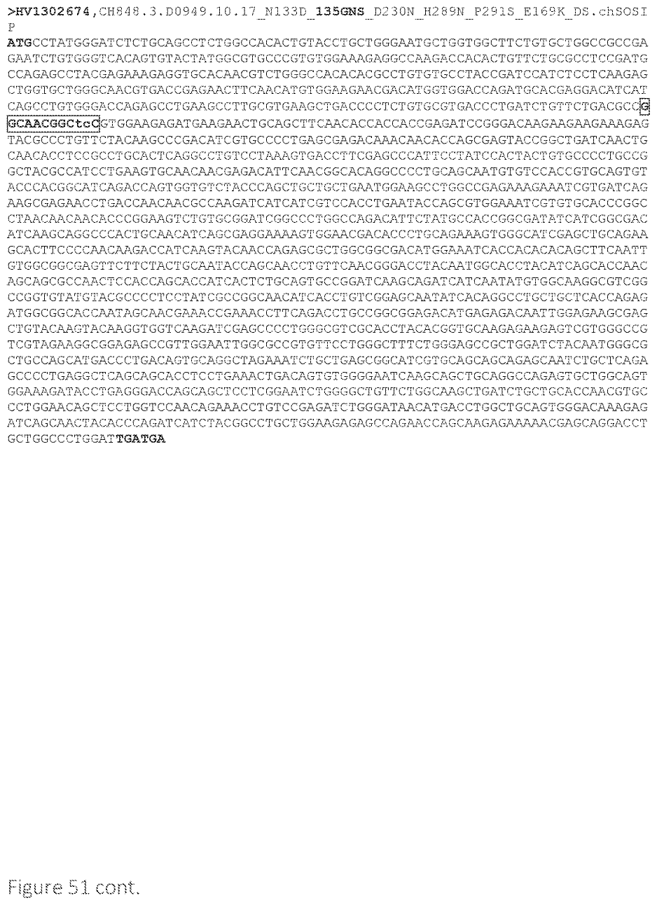

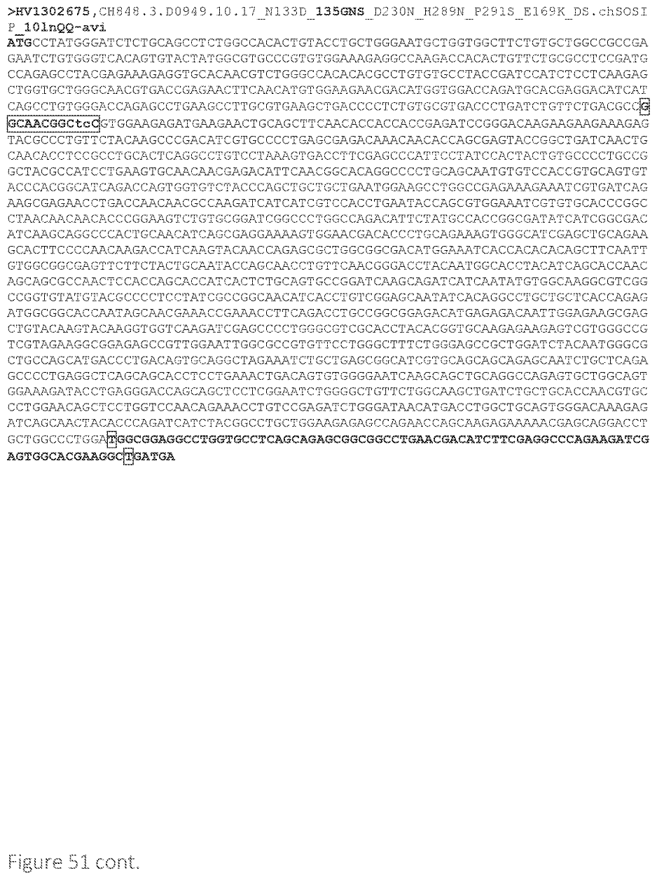

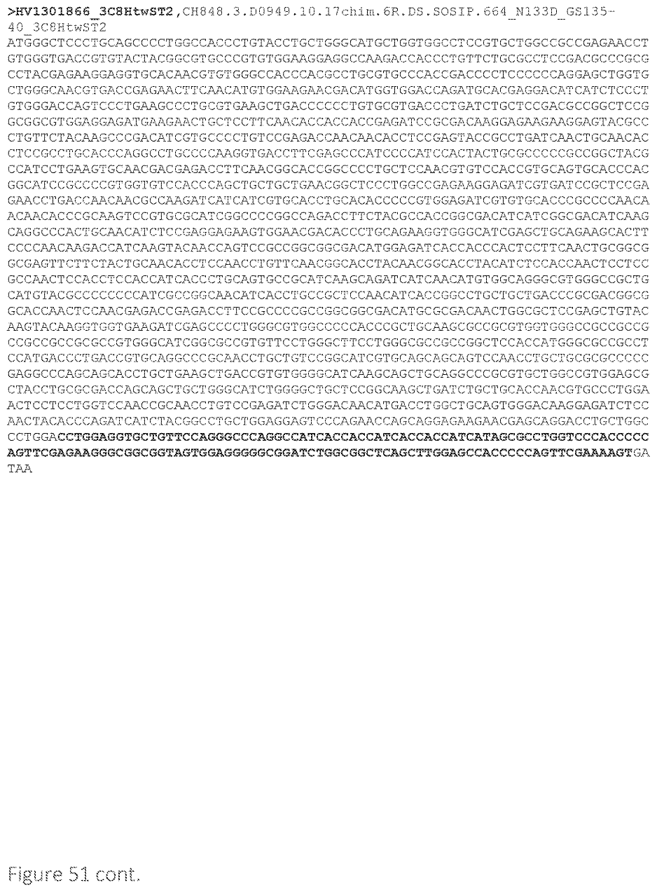

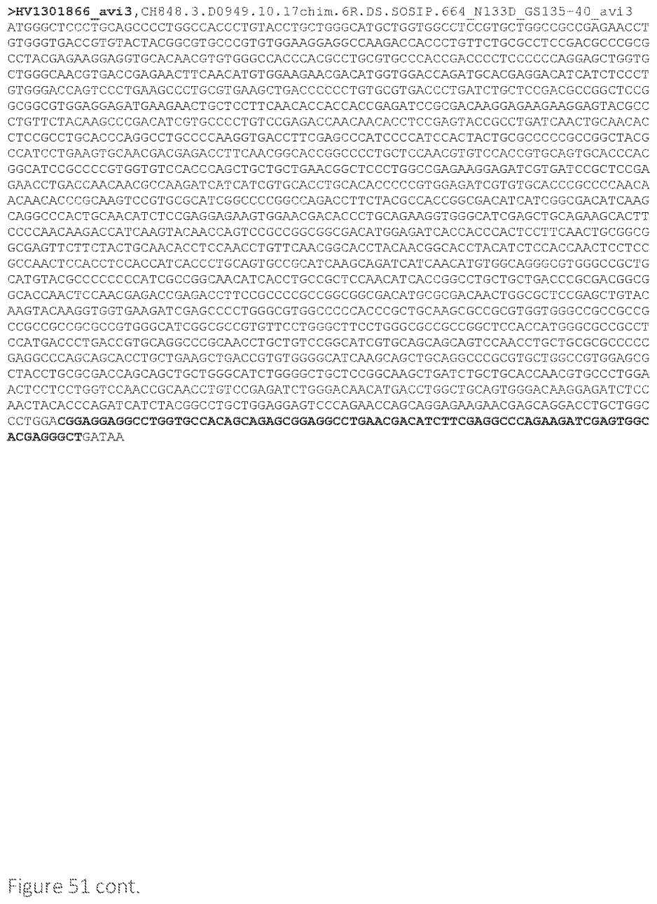

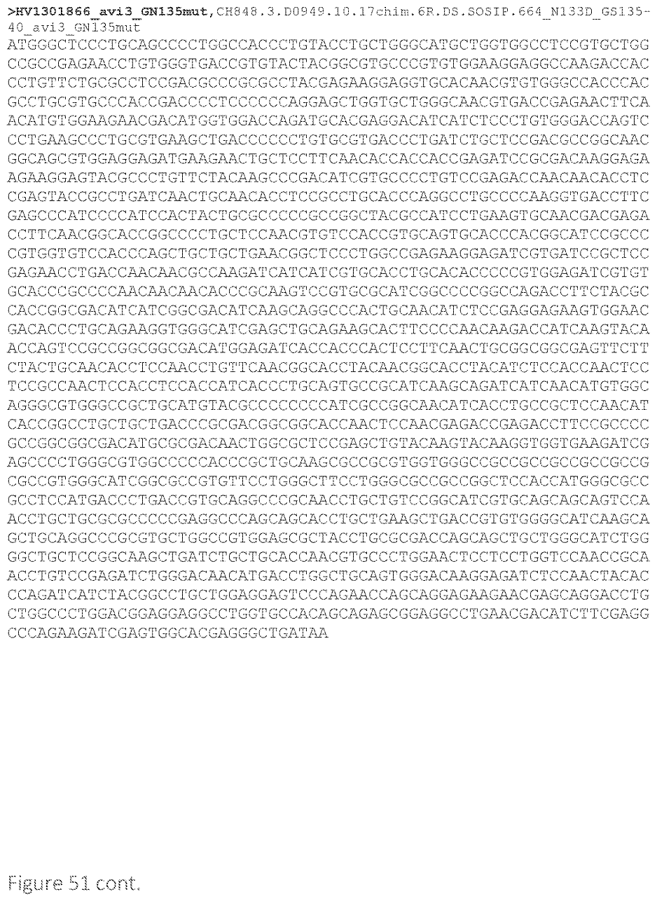

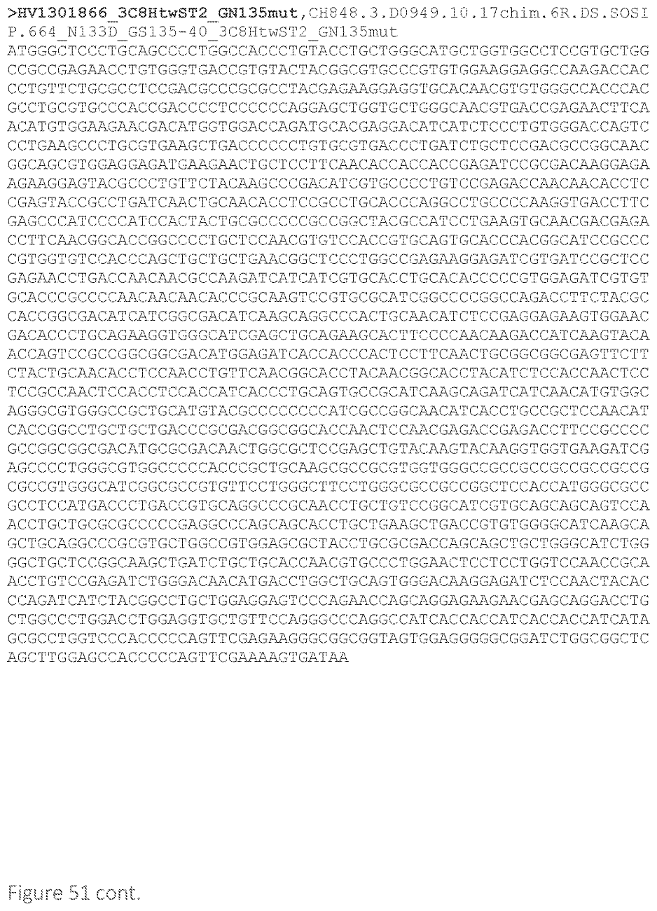

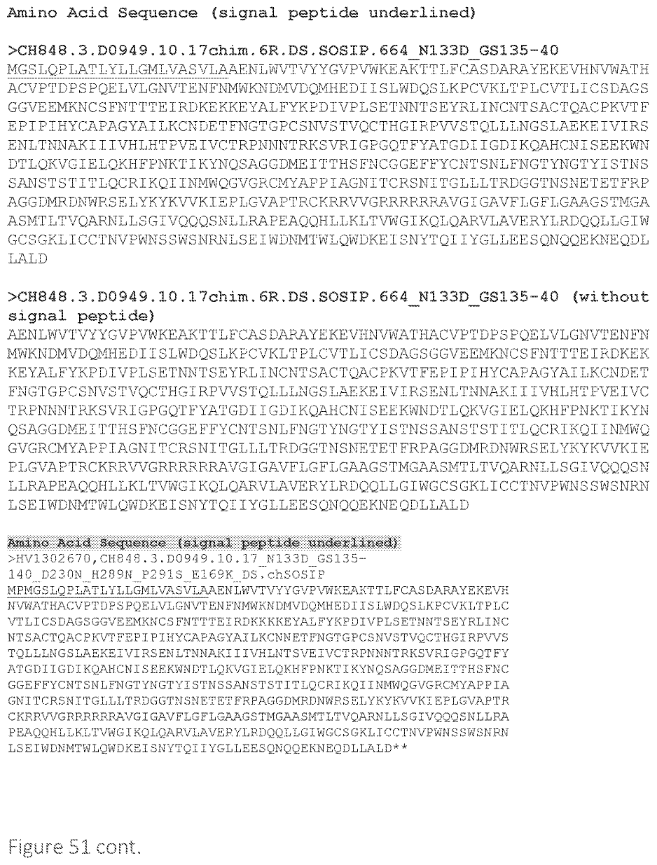

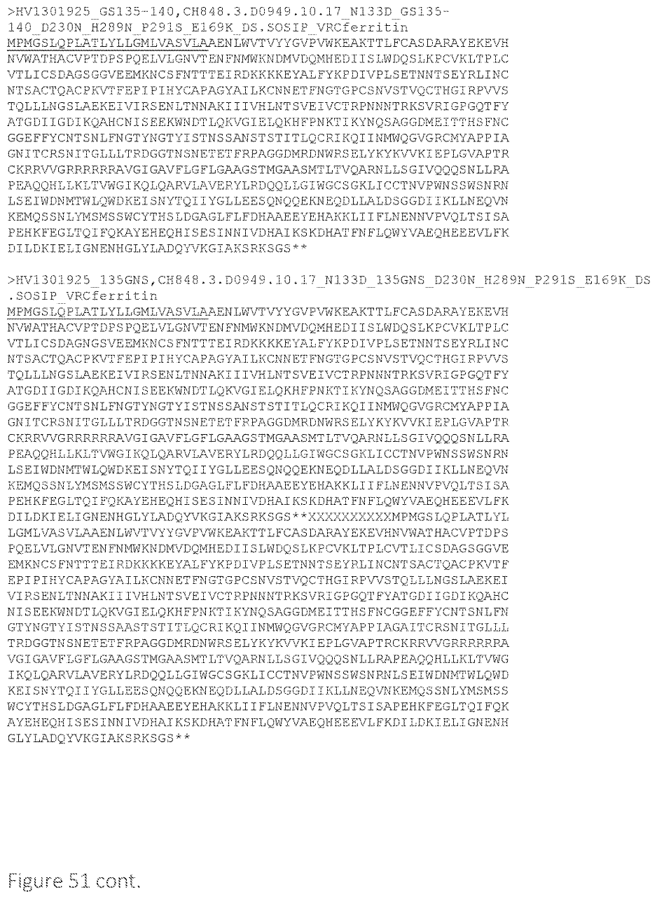

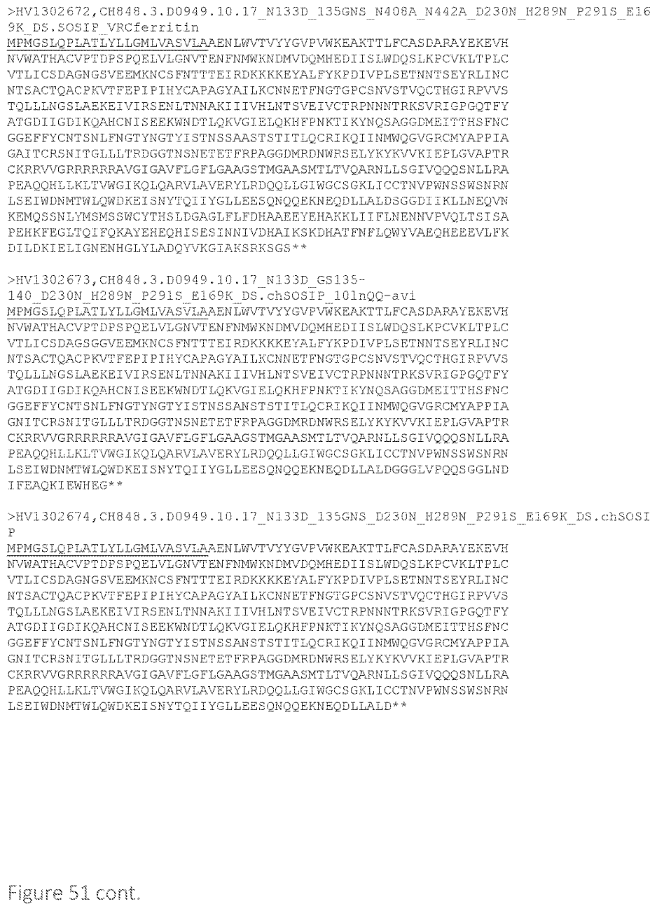

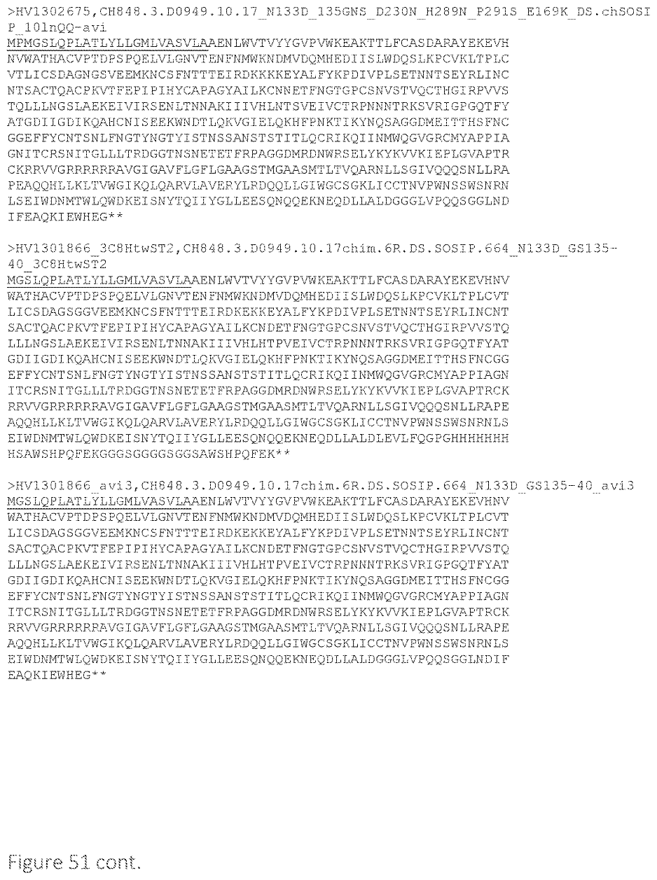

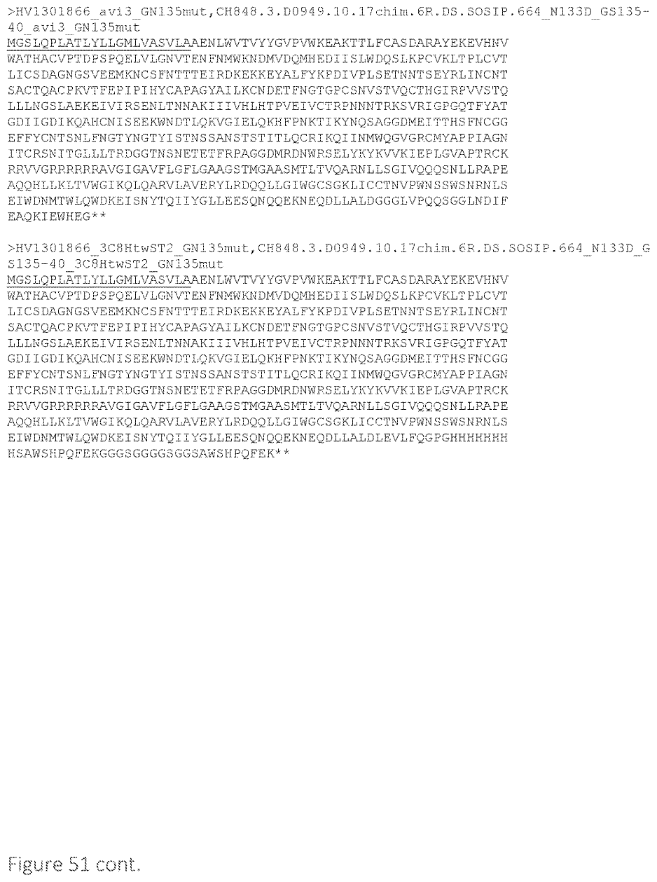

In certain embodiments the envelope is any of the forms of HIV-1 envelope. In certain embodiments the envelope is gp120, gp140, gp145 (i.e. with a transmembrane domain), or gp150. In certain embodiments, gp140 is designed to form a stable trimer. See Table 1, 2, FIGS. 41-51 for non-limiting examples of sequence designs. In certain embodiments envelope protomers form a trimer which is not a SOSIP timer. In certain embodiment the trimer is a SOSIP based trimer wherein each protomer comprises additional modifications. In certain embodiments, envelope trimers are recombinantly produced. In certain embodiments, envelope trimers are purified from cellular recombinant fractions by antibody binding and reconstituted in lipid comprising formulations. See for example WO2015/127108 titled “Trimeric HIV-1 envelopes and uses thereof” and US2020/0002383 which content is herein incorporated by reference in its entirety. In certain embodiments the envelopes of the invention are engineered and comprise non-naturally occurring modifications.

In certain embodiments, the envelope is in a liposome. In certain embodiments the envelope comprises a transmembrane domain with a cytoplasmic tail, wherein the transmembrane domain is embedded in a liposome. In certain embodiments, the nucleic acid comprises a nucleic acid sequence which encodes a gp120, gp140, gp145, gp150, or gp160.

In certain embodiments, where the nucleic acids are operably linked to a promoter and inserted in a vector, the vector is any suitable vector. In some embodiments, the vector comprises DNA. Non-limiting examples include, VSV, replicating rAdenovirus type 4, MVA, Chimp adenovirus vectors, pox vectors, and the like. In certain embodiments, the nucleic acids are administered in NanoTaxi block polymer nanospheres. In certain embodiments, the composition and methods comprise an adjuvant. Non-limiting examples include, 3M052, AS01 B, AS01 E, gla/SE, alum, Poly I poly C (poly IC), polyIC/long chain (LC) TLR agonists, TLR7/8 and 9 agonists, or a combination of TLR7/8 and TLR9 agonists (see Moody et al. (2014) J. Virol. March 2014 vol. 88, no. 6, 3329-3339), or any other adjuvant. Non-limiting examples of TLR7/8 agonist include TLR7/8 ligands, Gardiquimod, Imiquimod and R848 (resiquimod). A non-limiting embodiment of a combination of TLR7/8 and TLR9 agonist comprises R848 and oCpG in STS (see Moody et al. (2014) J. Virol. March 2014 vol. 88, no. 6, 3329-3339). In non-limiting embodiments, the adjuvant is an LNP. See e.g., without limitation Shirai et al. “Lipid Nanoparticle Acts as a Potential Adjuvant for Influenza Split Vaccine without Inducing Inflammatory Responses” Vaccines 2020, 8, 433; doi: 10.3390/vaccines8030433, published 3 Aug. 2020.

In non-limiting embodiments, LNPs used as adjuvants for proteins or mRNA compositions are composed of an ionizable lipid, cholesterol, lipid conjugated with polyethylene glycol, and a helper lipid. Non-limiting embodiments include LNPs without polyethylene glycol.

In certain aspects the invention provides a cell comprising a nucleic acid encoding any one of the envelopes of the invention suitable for recombinant expression. In certain aspects, the invention provides a clonally derived population of cells encoding any one of the envelopes of the invention suitable for recombinant expression. In certain aspects, the invention provides a stable pool of cells encoding any one of the envelopes of the invention suitable for recombinant expression.

In certain aspects, the invention provides a recombinant HIV-1 envelope polypeptide as described here, wherein the polypeptide is a non-naturally occurring protomer designed to form an envelope trimer. The invention also provides nucleic acids encoding these recombinant polypeptides. Non-limiting examples of amino acids and nucleic acids of such protomers are disclosed herein.

In certain aspects the invention provides a recombinant trimer comprising three identical protomers of an envelope. In certain aspects the invention provides an immunogenic composition comprising the recombinant trimer and a carrier, wherein the trimer comprises three identical protomers of an HIV-1 envelope as described herein. In certain aspects the invention provides an immunogenic composition comprising nucleic acid encoding these recombinant HIV-1 envelope and a carrier.

Described herein are nucleic acid and amino acid sequences of HIV-1 envelopes. The sequences for use as immunogens are in any suitable form. In certain embodiments, the described HIV-1 envelope sequences are gp160s. In certain embodiments, the described HIV-1 envelope sequences are gp 120s. Other sequences, for example but not limited to stable SOSIP trimer designs, gp145s, gp140s, both cleaved and uncleaved, gp 140 Envs with the deletion of the cleavage (C) site, fusion (F) and immunodominant (I) region in gp41—named as gp140ΔCFI (gp140CFI), gp140 Envs with the deletion of only the cleavage (C) site and fusion (F) domain—named as gp140ΔCF (gp140CF), gp140 Envs with the deletion of only the cleavage (C)—named gp140ΔC (gp140C) (See e.g. Liao et al. Virology 2006, 353, 268-282), gp150s, gp4Is, can be readily derived from the nucleic acid and amino acid gp 160 sequences. In certain embodiments the nucleic acid sequences are codon optimized for optimal expression in a host cell, for example a mammalian cell, a rBCG cell or any other suitable expression system.

An HIV-1 envelope has various structurally defined fragments/forms: gp160; gp140—including cleaved gp140 and uncleaved gp140 (gp140C), gp140CF, or gp140CFI; gp120 and gp41. A skilled artisan appreciates that these fragments/forms are defined not necessarily by their crystal structure, but by their design and bounds within the full length of the gp160 envelope. While the specific consecutive amino acid sequences of envelopes from different strains are different, the bounds and design of these forms are well known and characterized in the art.

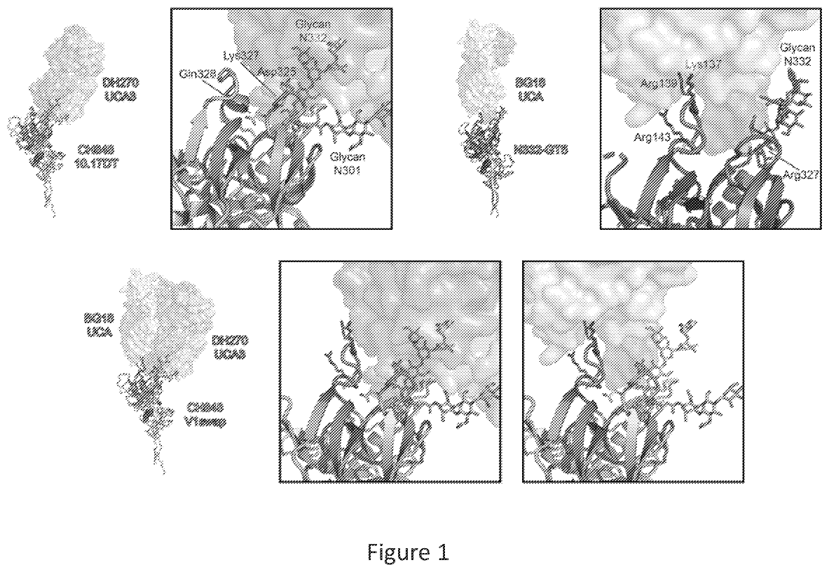

For example, it is well known in the art that during its transport to the cell surface, the gp 160 polypeptide is processed and proteolytically cleaved to gp 120 and gp41 proteins. Cleavages of gp160 to gp120 and gp41 occurs at a conserved cleavage site “REKR” (SEQ ID NO: 5). See Chakrabarti et al. Journal of Virology vol. 76, pp. 5357-5368 (2002); see, e.g., FIG. 1, and second paragraph in the Introduction on p. 5357; Binley et al. Journal of Virology vol. 76, pp. 2606-2616 (2002) for example at Abstract; Gao et al. Journal of Virology vol. 79, pp. 1154-1163 (2005); Liao et al. Virology vol. 353 (2): 268-282 (2006).

The role of the furin cleavage site was well understood both in terms of improving cleavage efficiency, see Binley et al. supra, and eliminating cleavage, see Bosch and Pawlita, Virology 64 (5): 2337-2344 (1990); Guo et al. Virology 174:217-224 (1990); McCune et al. Cell 53:55-67 (1988); Liao et al. J Virol. April;87 (8): 4185-201 (2013).

Likewise, the design of gp 140 envelope forms is also well known in the art, along with the various specific changes which give rise to the gp140C (uncleaved envelope), gp140CF and gp140CFI forms. Envelope gp140 forms are designed by introducing a stop codon within the gp41 sequence. See Chakrabarti et al. at FIG. 1.

Envelope gp140C refers to a gp 140 HIV-1 envelope design with a functional deletion of the cleavage (C) site, so that the gp 140 envelope is not cleaved at the furin cleavage site. The specification describes cleaved and uncleaved forms, and various furin cleavage site modifications that prevent envelope cleavage are known in the art. In some embodiments of the gp 140C form, two of the R residues in and near the furin cleavage site are changed to E, e.g., RRVVEREKR (SEQ ID NO: 6) is changed to ERVVEREKE (SEQ ID NO: 7), and is one example of an uncleaved gp140 form. Another example is the gp 140C form which has the REKR site (SEQ ID NO: 5) changed to SEKS (SEQ ID NO: 8). See supra for references.

Envelope gp140CF refers to a gp140 HIV-1 envelope design with a deletion of the cleavage (C) site and fusion (F) region. Envelope gp 140CFI refers to a gp 140 HIV-1 envelope design with a deletion of the cleavage (C) site, fusion (F) and immunodominant (I) region in gp41. See Chakrabarti et al. Journal of Virology vol. 76, pp. 5357-5368 (2002) at for example FIG. 1, and Second paragraph in the Introduction on p. 5357; Binley et al. Journal of Virology vol. 76, pp. 2606-2616 (2002) for example at Abstract; Gao et al. Journal of Virology vol. 79, pp. 1154-1163 (2005); Liao et al. Virology vol. 353 (2): 268-282 (2006).

In certain embodiments, the envelope design in accordance with the present invention involves deletion of residues (e.g., 5-11, 5, 6, 7, 8, 9, 10, or 11 amino acids) at the N-terminus. For delta N-terminal design, amino acid residues ranging from 4 residues or even fewer to 14 residues or even more are deleted. These residues are between the maturation (signal peptide, usually ending with CXX, wherein X can be any amino acid) and “VPVXXXX . . . ”. In case of CH505 T/F Env as an example, 8 amino acids (italicized and underlined in the below sequence) were deleted:

(SEQ ID NO: 9)
MRVMGIQRNYPQWWIWSMLGFWMLMICNGMWVTVYYGVPVWKEAKTTLFCASDA
KAYEKEVHNVWATHACVPTDPNPQE...(rest of envelope sequence is indicated as “...”).

In other embodiments, the delta N-design described for CH505 T/F envelope can be used to make delta N-designs of other envelopes. In certain embodiments, the invention relates generally to an HIV-1 envelope immunogen, gp160, gp120, or gp140, without an N-terminal Herpes Simplex gD tag substituted for amino acids of the N-terminus of gp120, with an HIV leader sequence (or other leader sequence), and without the original about 4 to about 25, for example 4, 5, 6, 7, 8, 9, 10, 11, 12, 13, 14, 15, 16, 17, 18, 19, 20, 21, 22, 23, 24, 25 amino acids of the N-terminus of the envelope (e.g. gp120). See US2014/0248311, e.g. at paragraphs [0043]-[0050], the contents of which publication is hereby incorporated by reference in its entirety.

The general strategy of deletion of N-terminal amino acids of envelopes results in proteins, for example gp 120s, expressed in mammalian cells that are primarily monomeric, as opposed to dimeric, and, therefore, solves the production and scalability problem of commercial gp120 Env vaccine production. In other embodiments, the amino acid deletions at the N-terminus result in increased immunogenicity of the envelopes.

In certain aspects, the invention provides composition and methods which use a selection of Envs, as gp120s, gp140s cleaved and uncleaved, gp145s, gp150s and gp160s, stabilized and/or multimerized trimers, as proteins, DNA, RNAs, or any combination thereof, administered as primes and boosts to elicit an immune response. Envs as proteins could be co-administered with nucleic acid vectors containing Envs to amplify antibody induction. In certain embodiments, the compositions and methods include any immunogenic HIV-1 sequences to give the best coverage for T cell help and cytotoxic T cell induction. In certain embodiments, the compositions and methods include mosaic and/or consensus HIV-1 genes to give the best coverage for T cell help and cytotoxic T cell induction. In certain embodiments, the compositions and methods include mosaic group M and/or consensus genes to give the best coverage for T cell help and cytotoxic T cell induction. In some embodiments, the mosaic genes are any suitable gene from the HIV-1 genome. In some embodiments, the mosaic genes are Env genes, Gag genes, Pol genes, Nef genes, or any combination thereof. See e.g., U.S. Pat. No. 7,951,377. In some embodiments the mosaic genes are bivalent mosaics. In some embodiments the mosaic genes are trivalent. In some embodiments, the mosaic genes are administered in a suitable vector with each immunization with Env gene inserts in a suitable vector and/or as a protein. In some embodiments, the mosaic genes, for example as bivalent mosaic Gag group M consensus genes, are administered in a suitable vector, for example but not limited to HSV2, would be administered with each immunization with Env gene inserts in a suitable vector, for example but not limited to HSV-2.

In certain aspects the invention provides compositions and methods of Env genetic immunization either alone or with Env proteins to recreate the swarms of evolved viruses that have led to bnAb induction. Nucleotide-based vaccines offer a flexible vector format to immunize against virtually any protein antigen. Currently, two types of genetic vaccination are available for testing—DNAs and mRNAs.

In certain aspects the invention contemplates using immunogenic compositions wherein immunogens are delivered as DNA. See Graham B S, Enama M E, Nason M C, Gordon I J, Peel S A, et al. (2013) DNA Vaccine Delivered by a Needle-Free Injection Device Improves Potency of Priming for Antibody and CD8+ T-Cell Responses after rAd5 Boost in a Randomized Clinical Trial. PLOS ONE 8 (4): e59340, page 9. Various technologies for delivery of nucleic acids, as DNA and/or RNA, so as to elicit immune response, both T-cell and humoral responses, are known in the art and are under developments. In certain embodiments, DNA can be delivered as naked DNA. In certain embodiments, DNA is formulated for delivery by a gene gun. In certain embodiments, DNA is administered by electroporation, or by a needle-free injection technologies, for example but not limited to Biojector® device. In certain embodiments, the DNA is inserted in vectors. The DNA is delivered using a suitable vector for expression in mammalian cells. In certain embodiments the nucleic acids encoding the envelopes are optimized for expression. In certain embodiments DNA is optimized, e.g. codon optimized, for expression. In certain embodiments the nucleic acids are optimized for expression in vectors and/or in mammalian cells. In non-limiting embodiments these are bacterially derived vectors, adenovirus based vectors, rAdenovirus (e.g. Barouch D H, et al. Nature Med. 16:319-23, 2010), recombinant mycobacteria (e.g. rBCG or M smegmatis) (Yu, J S et al. Clinical Vaccine Immunol. 14:886-093,2007; ibid 13:1204-11,2006), and recombinant vaccinia type of vectors (Santra S. Nature Med. 16:324-8, 2010), for example but not limited to ALVAC, replicating (Kibler K V et al., PLOS One 6: e25674, 2011 Nov. 9.) and non-replicating (Perreau M et al. J. virology 85:9854-62, 2011) NYVAC, modified vaccinia Ankara (MVA)), adeno-associated virus, Venezuelan equine encephalitis (VEE) replicons, Herpes Simplex Virus vectors, and other suitable vectors.

In certain aspects the invention contemplates using immunogenic compositions wherein immunogens are delivered as DNA or RNA in suitable formulations. Various technologies which contemplate using DNA or RNA, or may use complexes of nucleic acid molecules and other entities to be used in immunization. In certain embodiments, DNA or RNA is administered as nanoparticles consisting of low dose antigen-encoding DNA formulated with a block copolymer (amphiphilic block copolymer 704). See Cany et al., Journal of Hepatology 2011 vol. 54 j 115-121; Amaoty et al., Chapter 17 in Yves Bigot (ed.), Mobile Genetic Elements: Protocols and Genomic Applications, Methods in Molecular Biology, vol. 859, pp293-305 (2012); Amaoty et al. (2013) Mol Genet Genomics. 2013 August;288 (7-8): 347-63. Nanocarrier technologies called Nanotaxi® for immunogenic macromolecules (DNA, RNA, Protein) delivery are under development. See for example technologies developed by In-Cell-Art.

In certain aspects, the invention provides nucleic acids comprising sequences encoding envelopes of the invention. In certain embodiments, the nucleic acids are DNAs. In certain embodiments, the nucleic acids are mRNAs. In certain aspects, the invention provides expression vectors comprising the nucleic acids of the invention.

In certain aspects, the invention provides a pharmaceutical composition comprising mRNAs encoding the inventive envelopes. In certain embodiments, these are optionally formulated in lipid nanoparticles (LNPs). In certain embodiments, the mRNAs are modified. Modifications include without limitations modified ribonucleotides, poly-A tail, 5′cap.

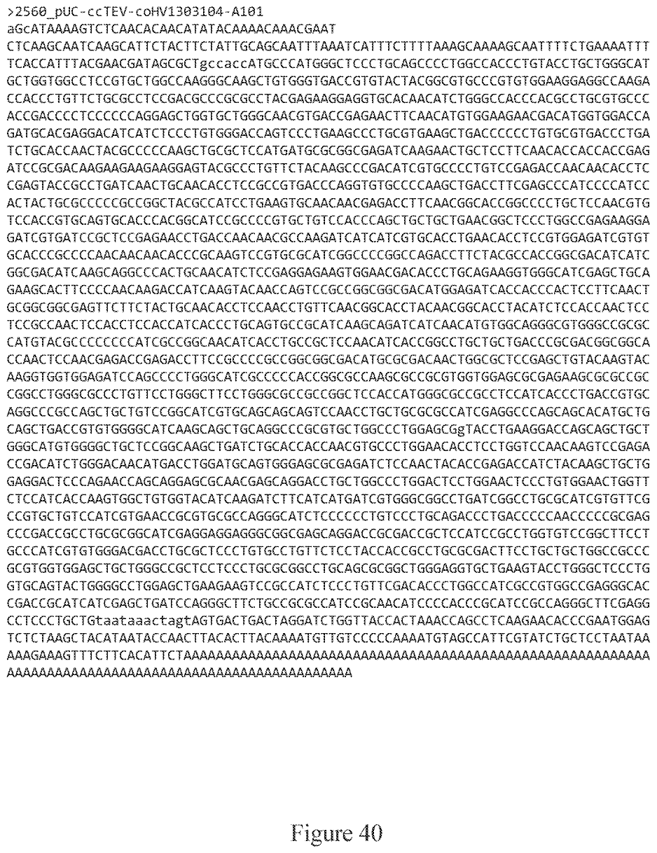

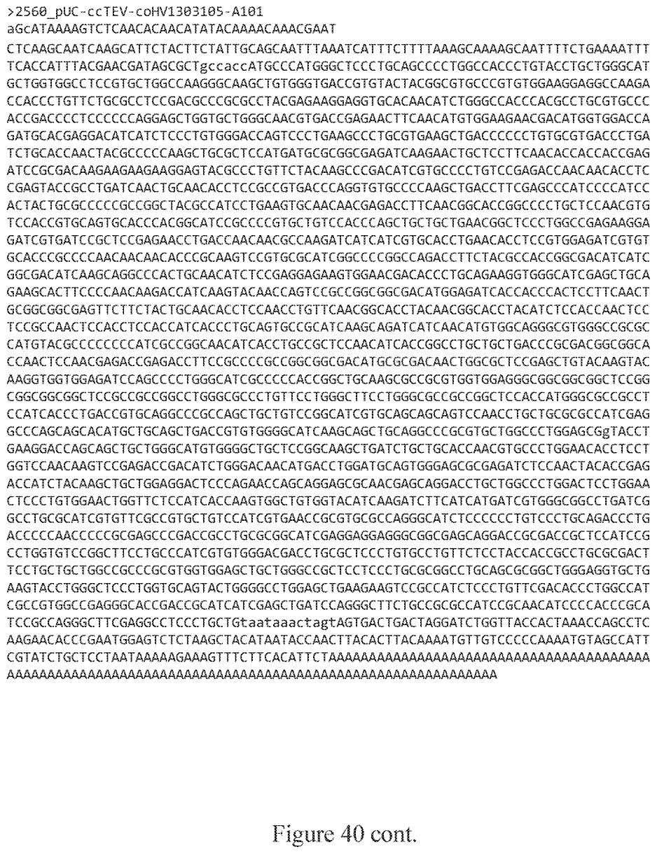

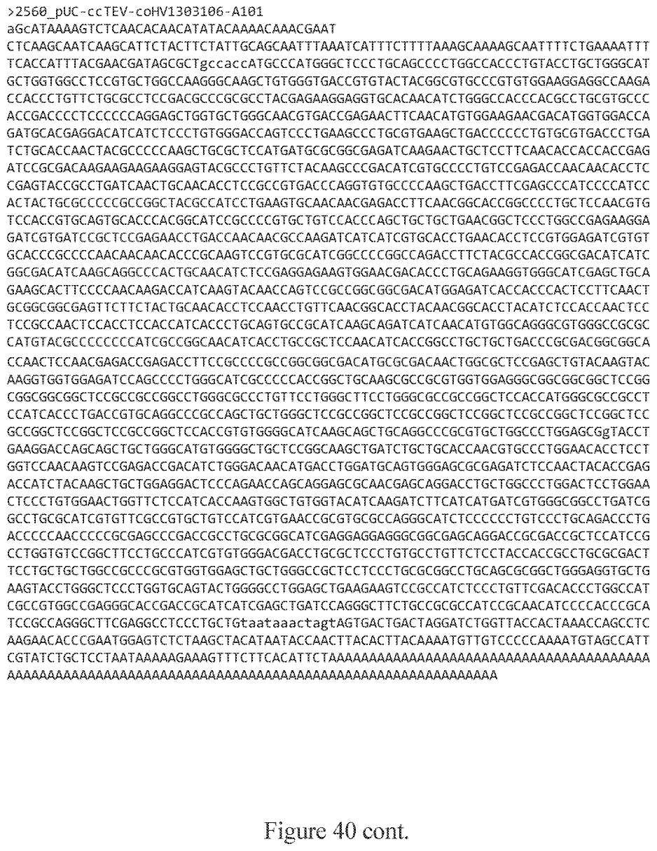

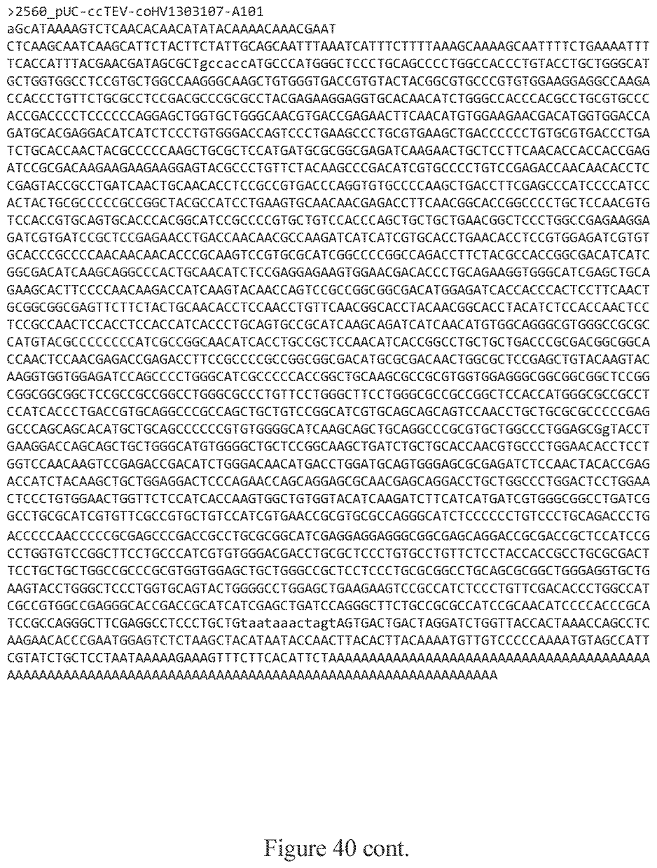

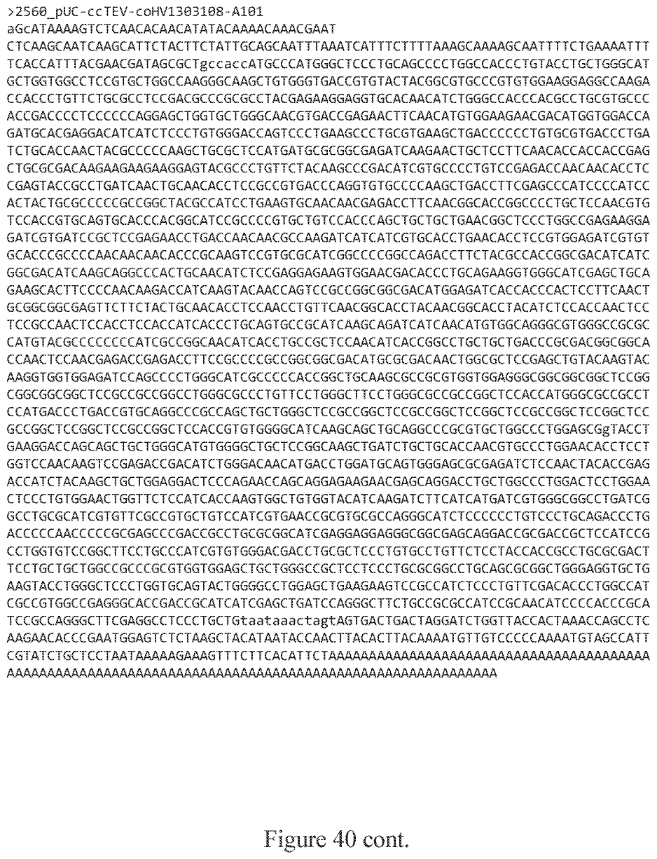

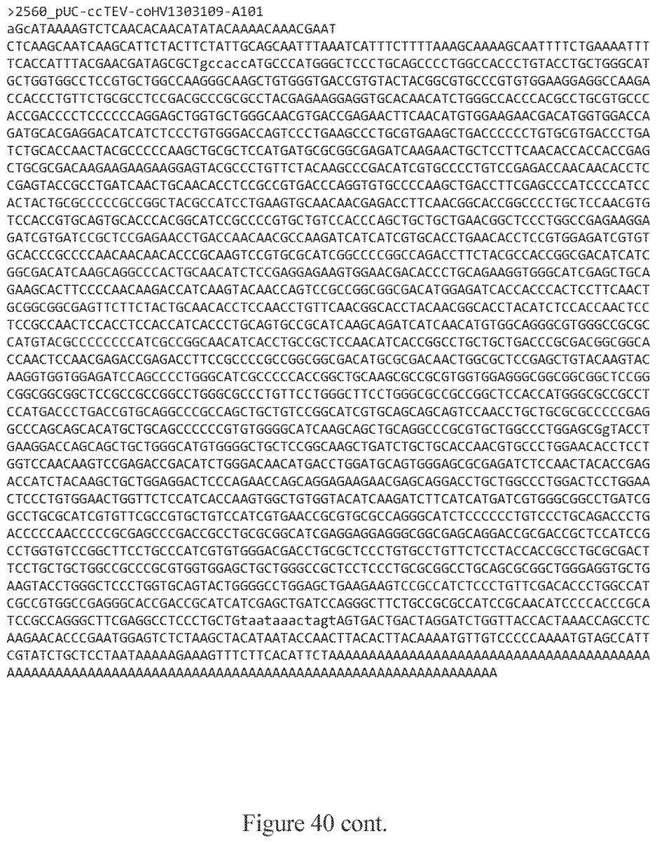

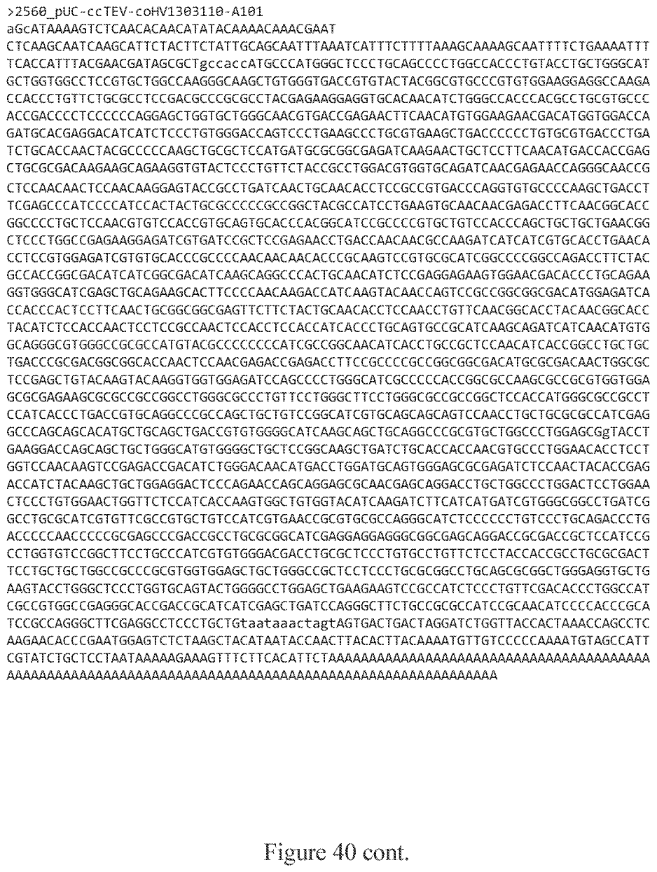

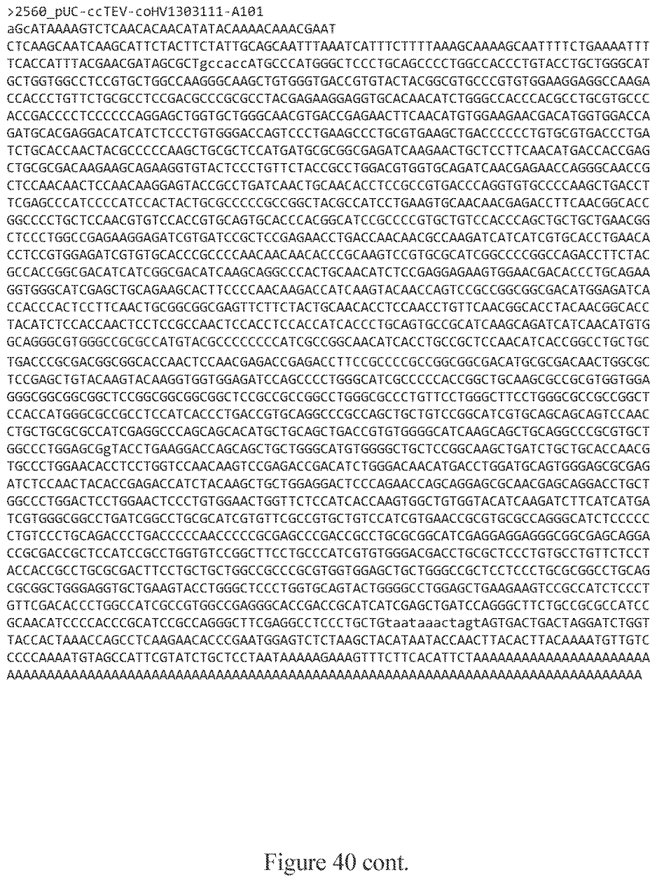

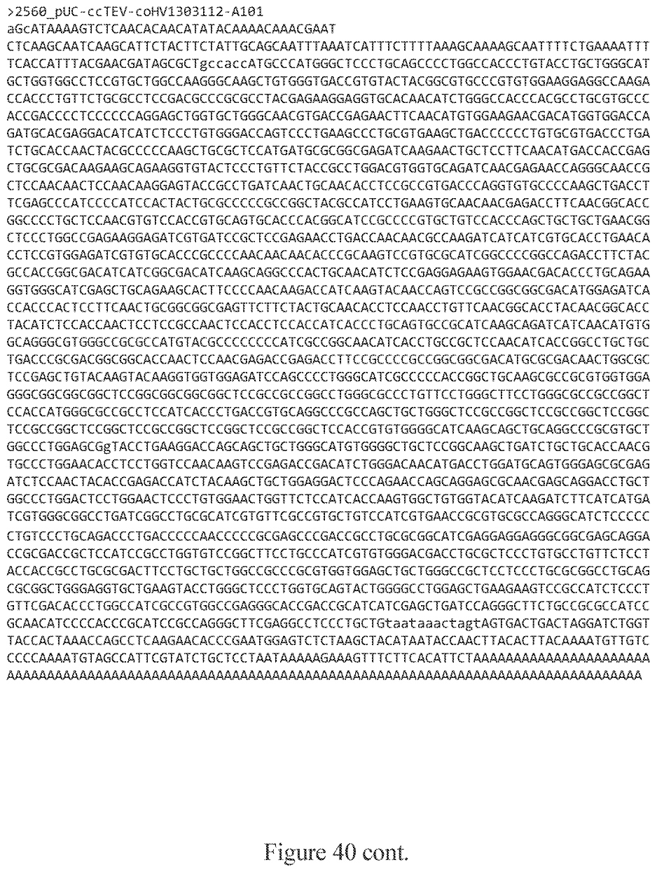

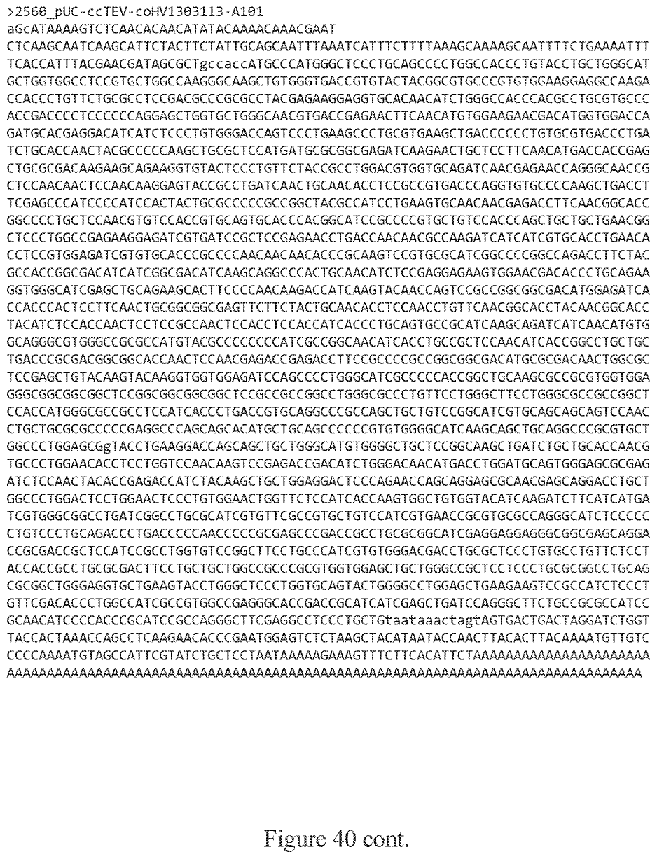

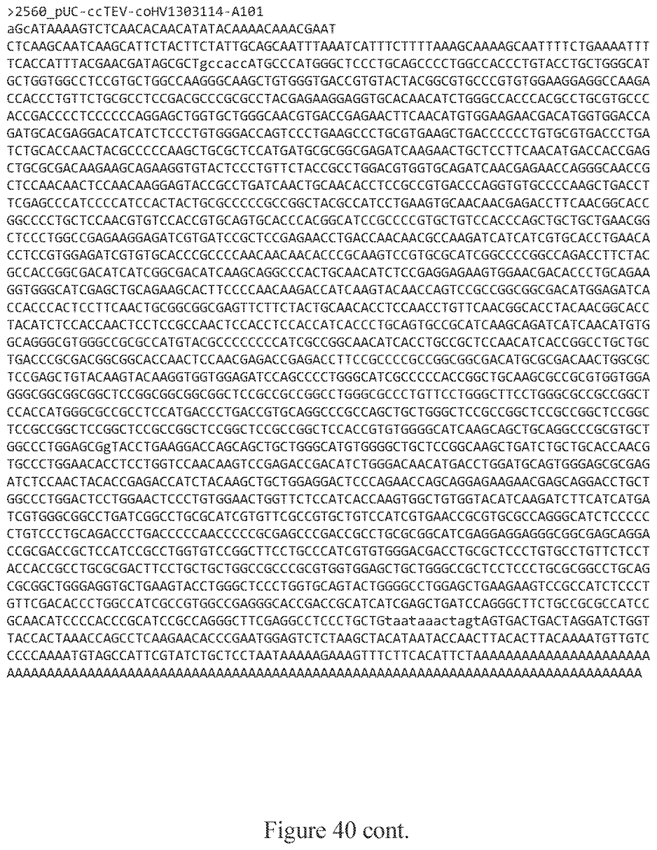

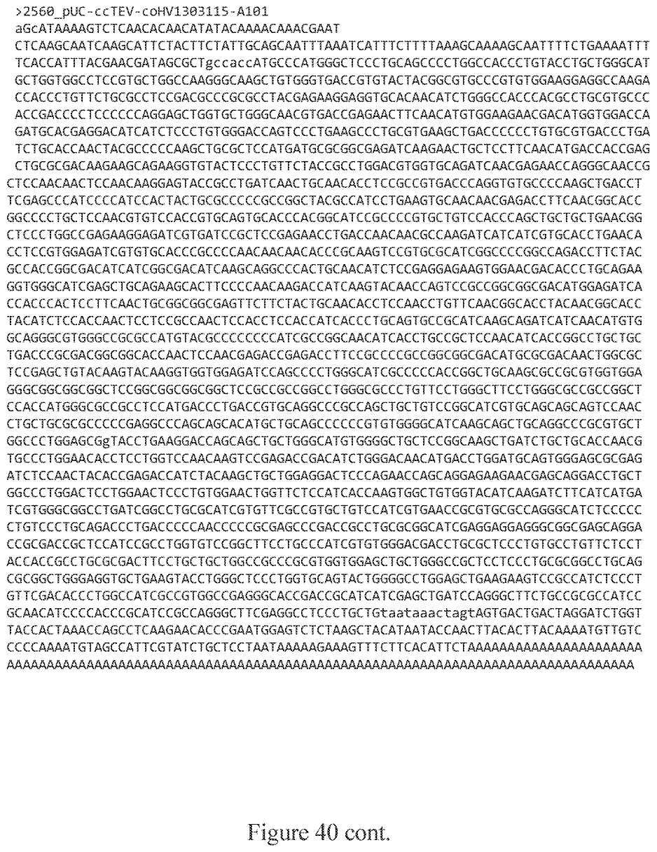

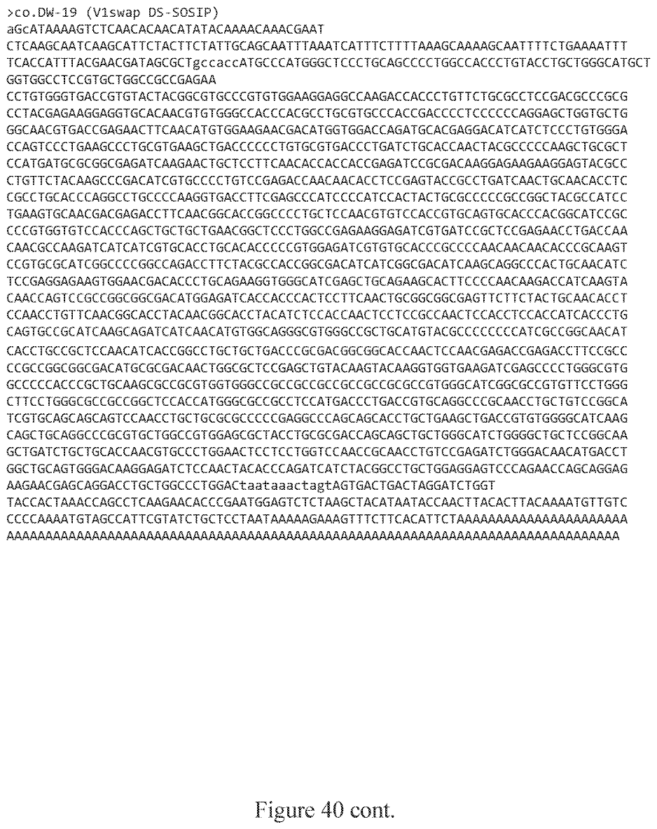

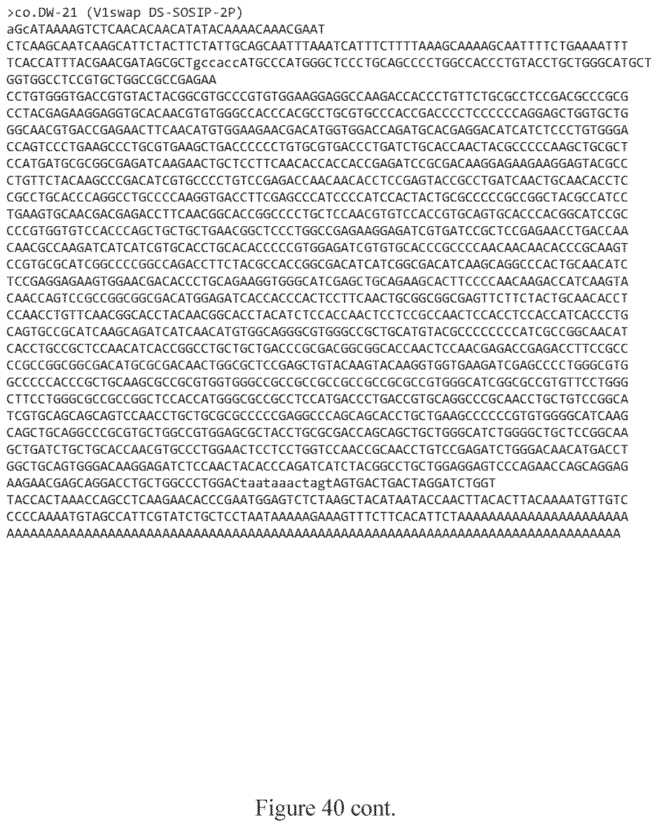

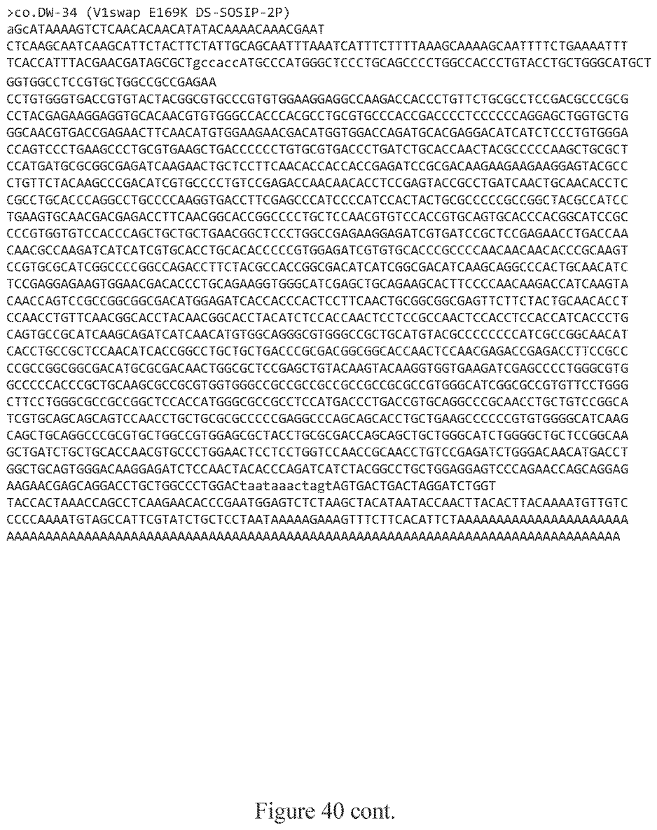

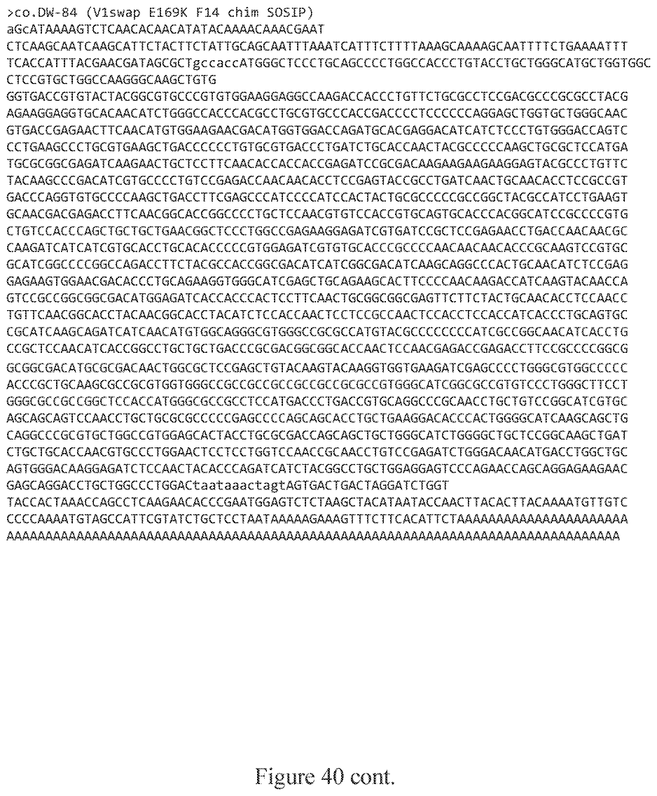

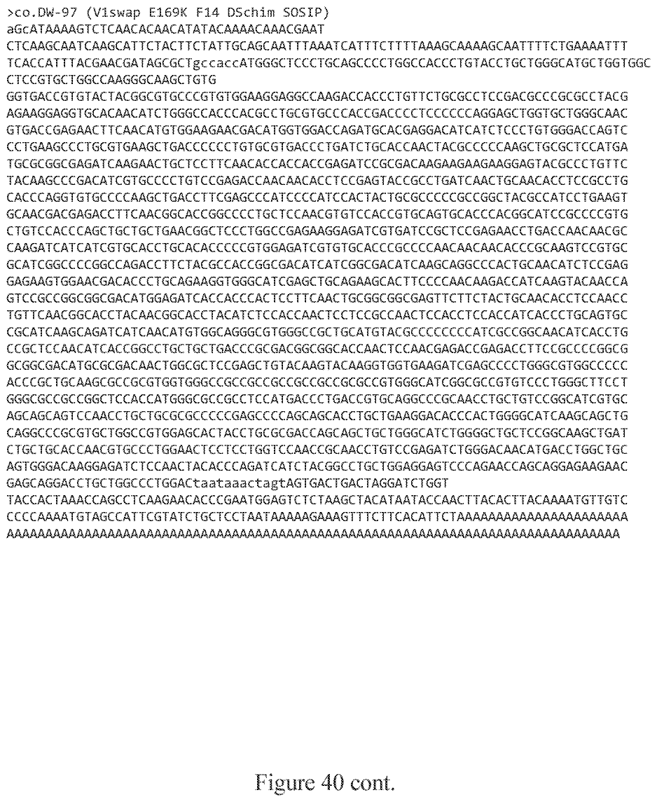

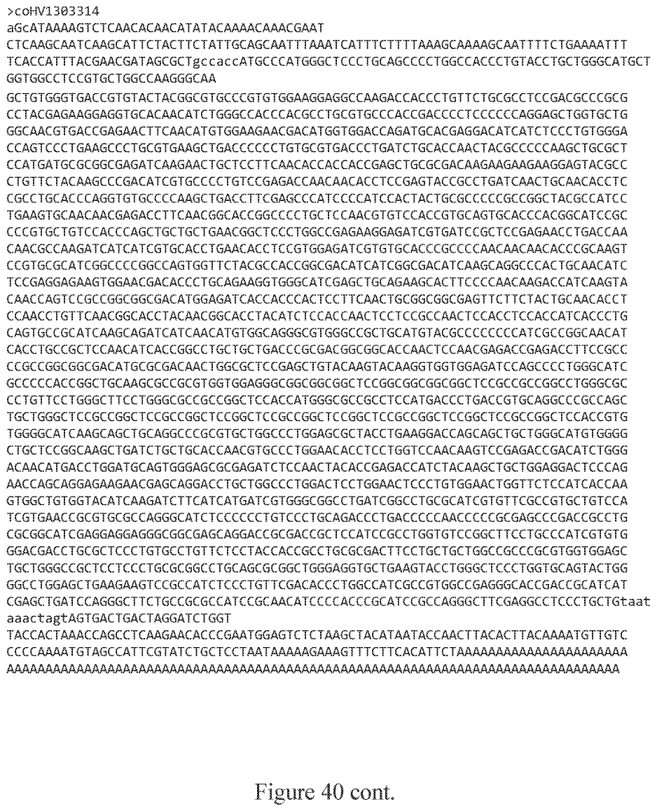

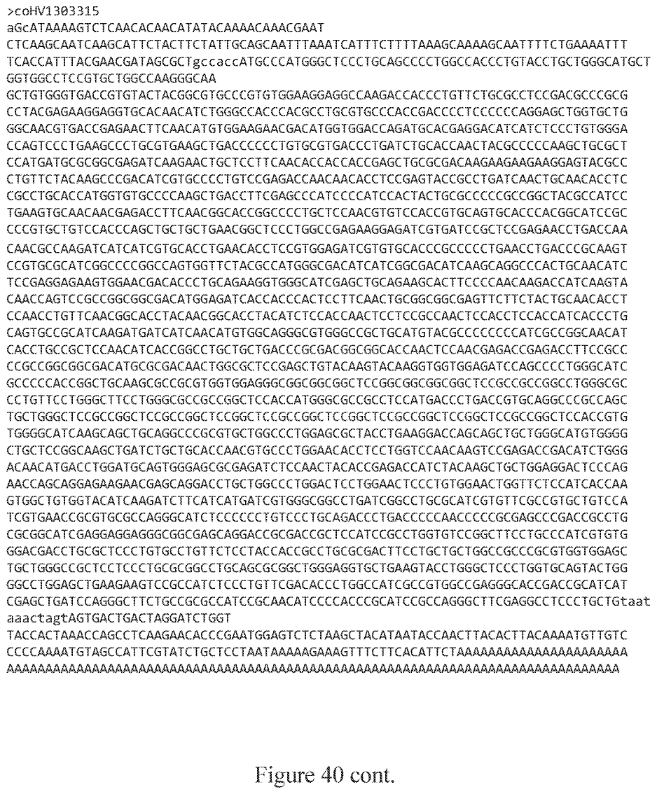

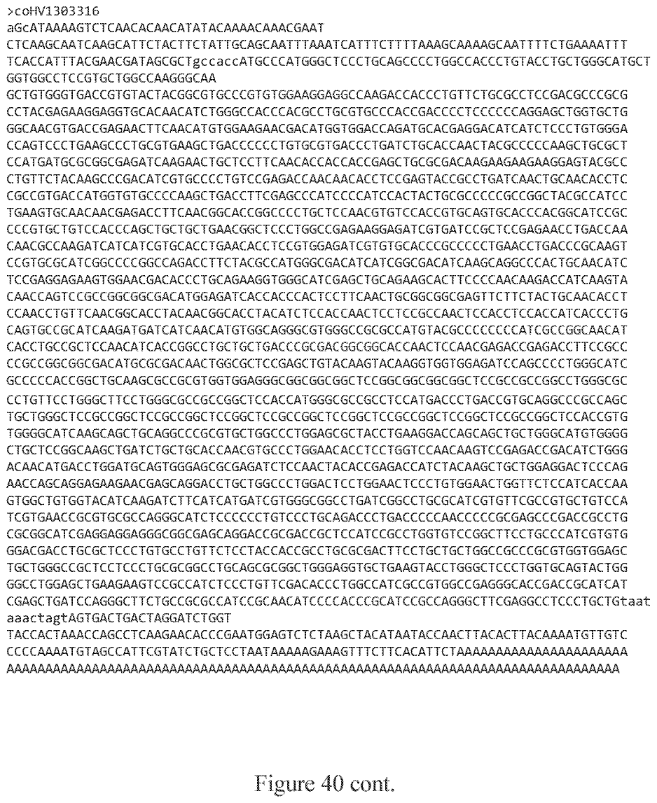

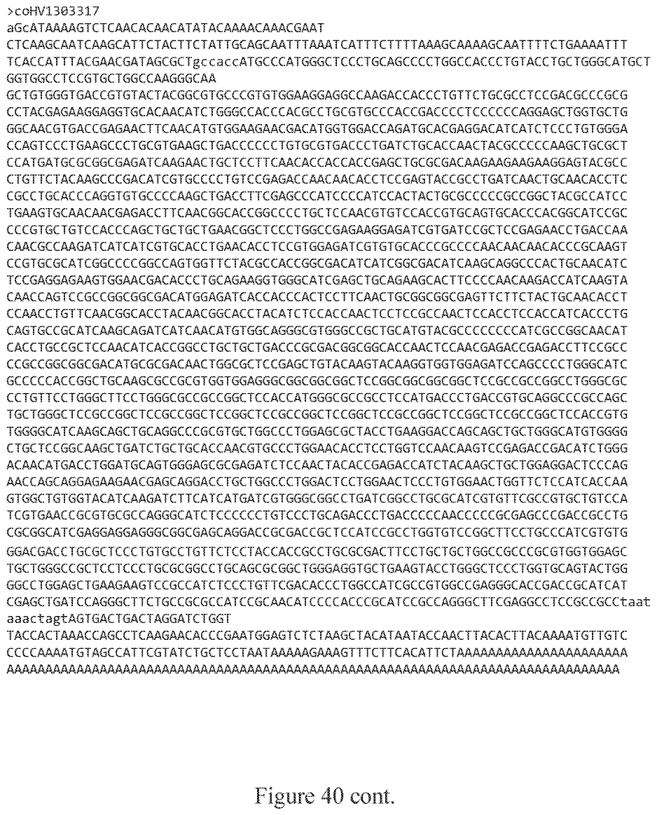

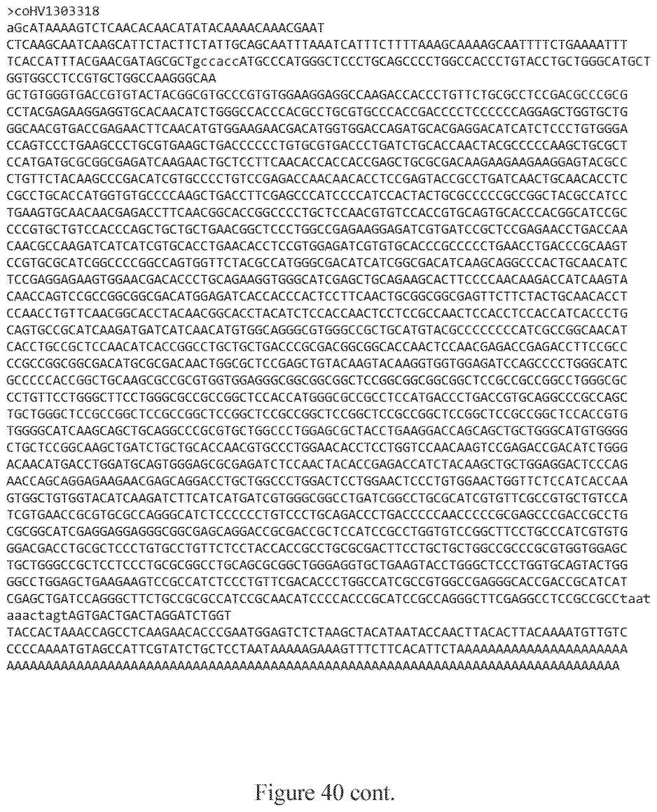

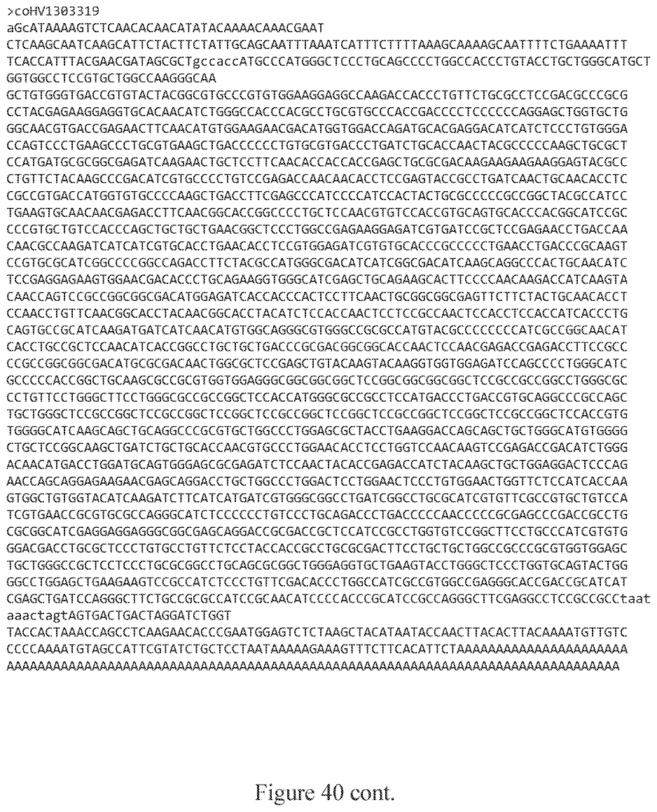

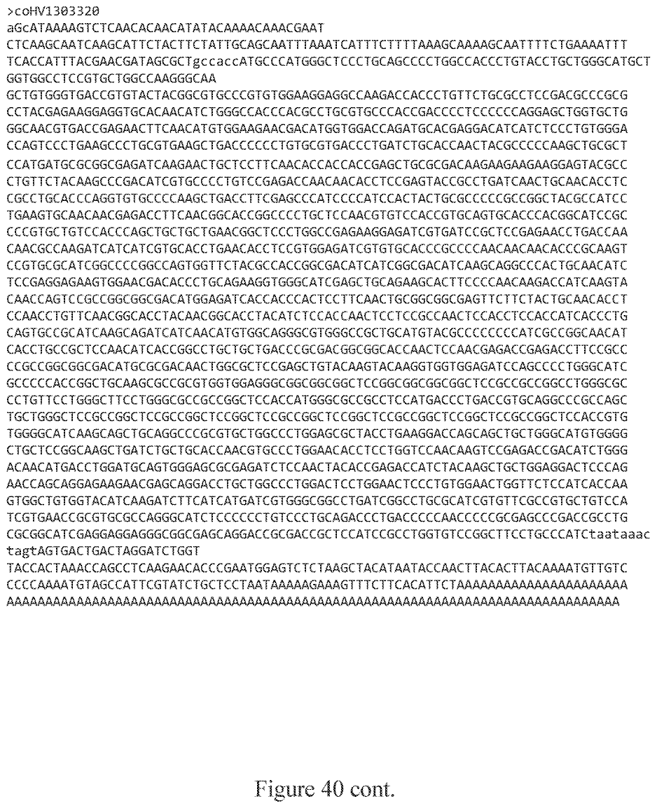

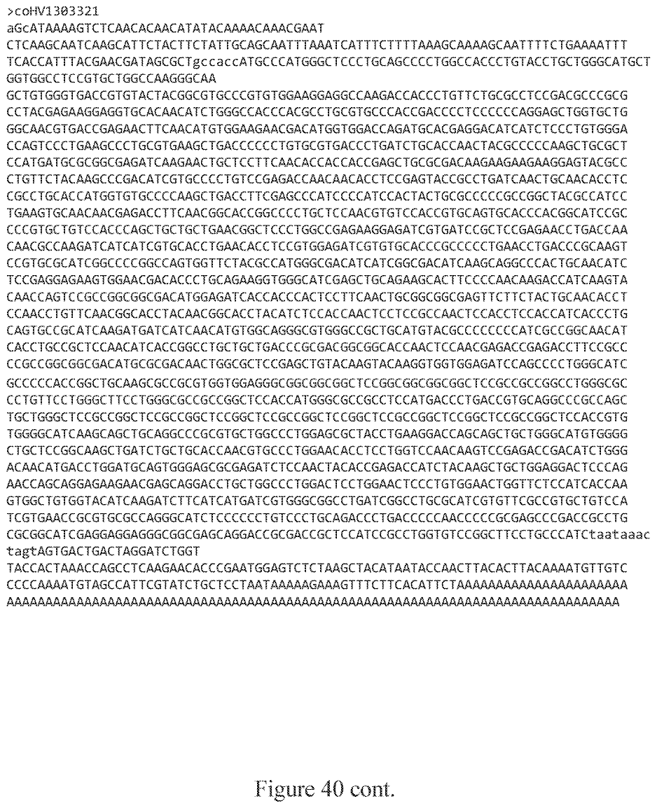

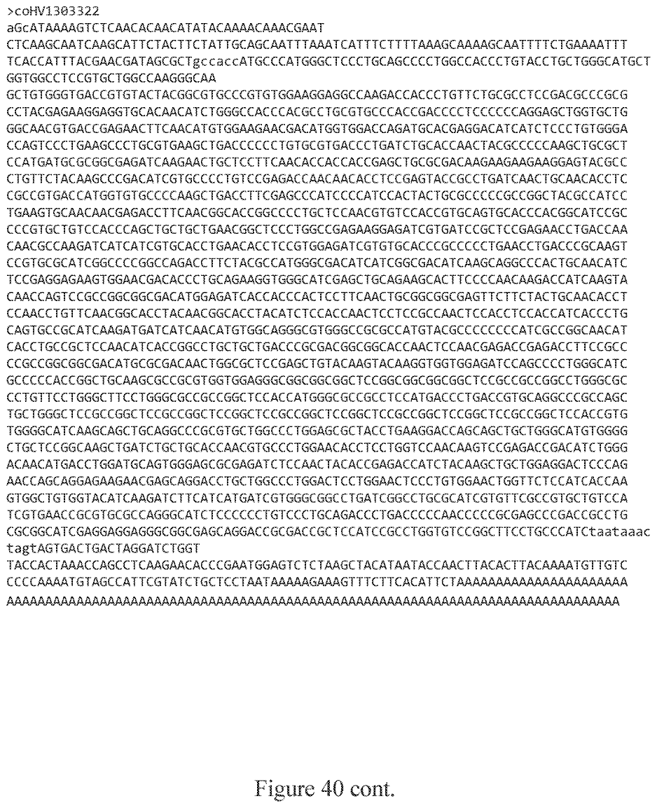

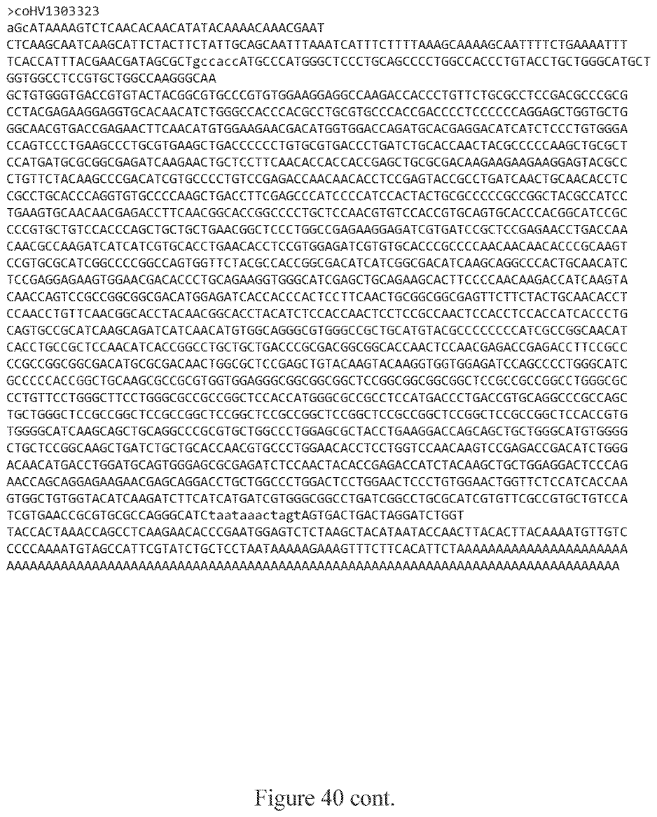

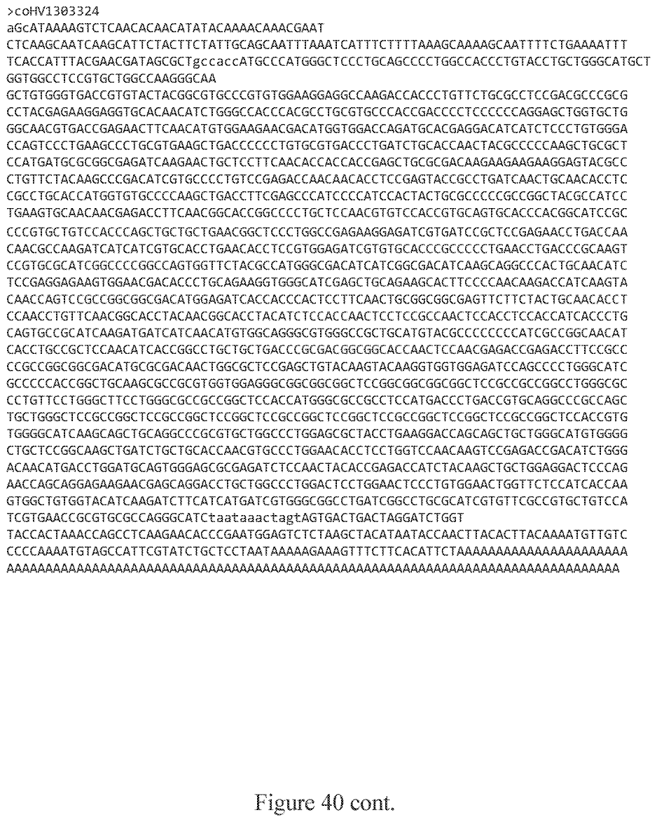

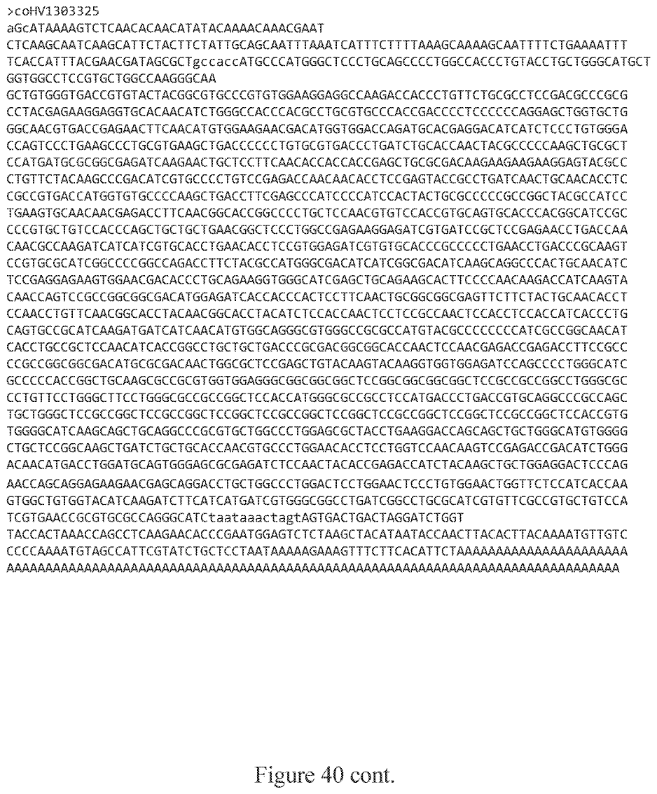

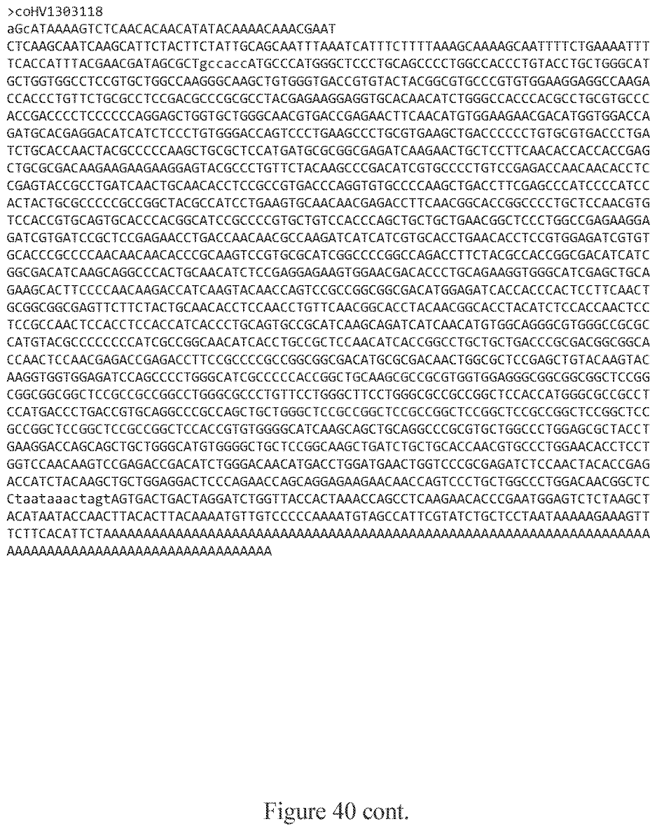

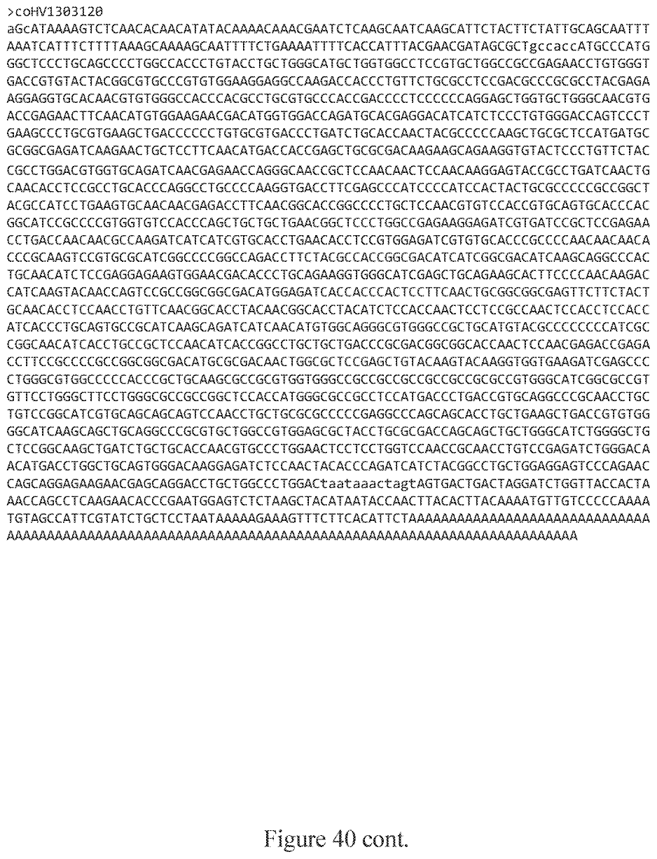

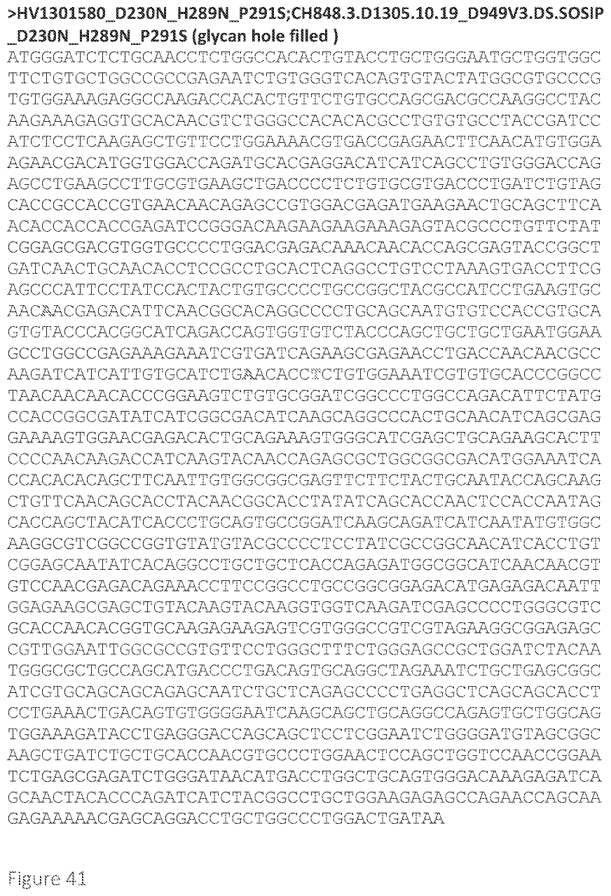

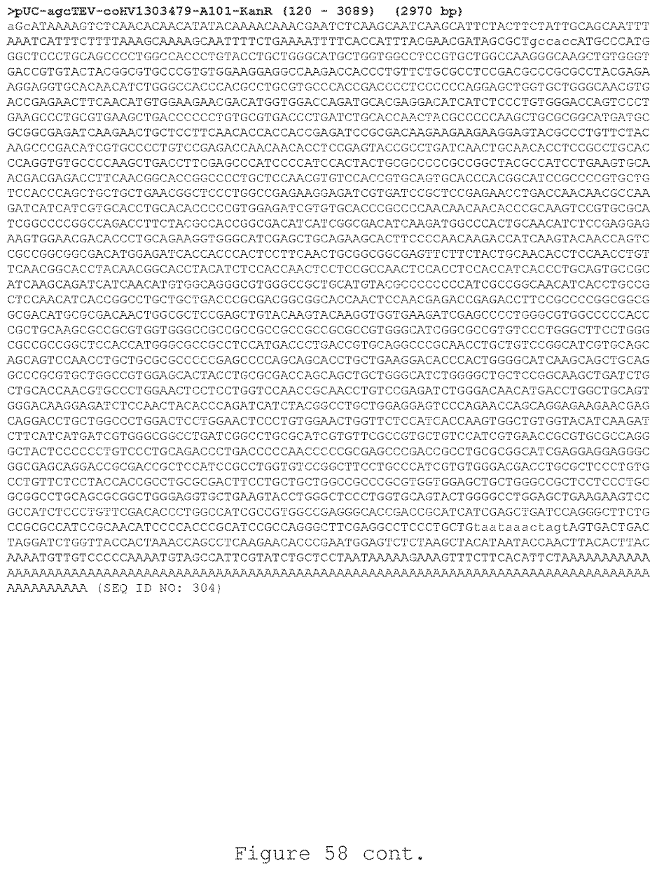

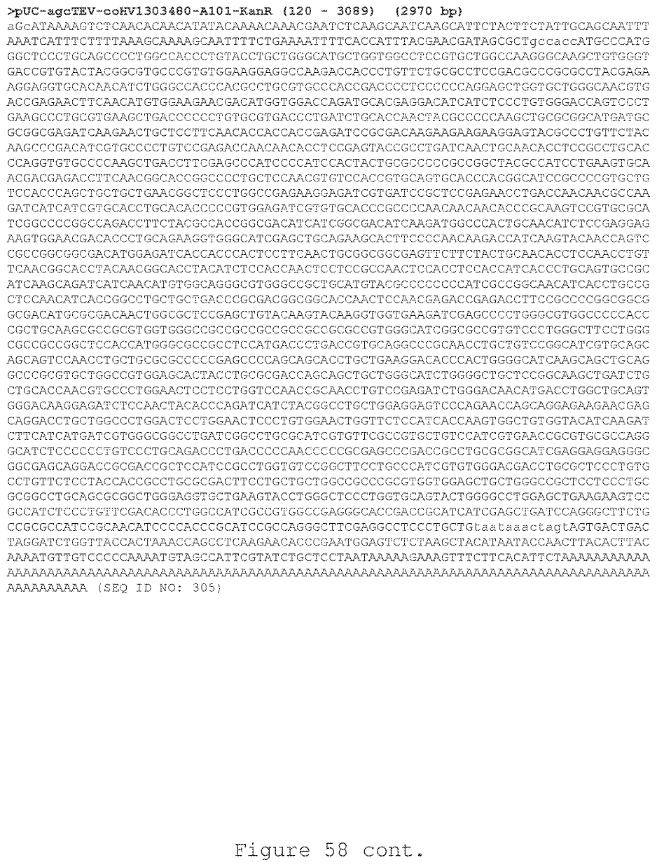

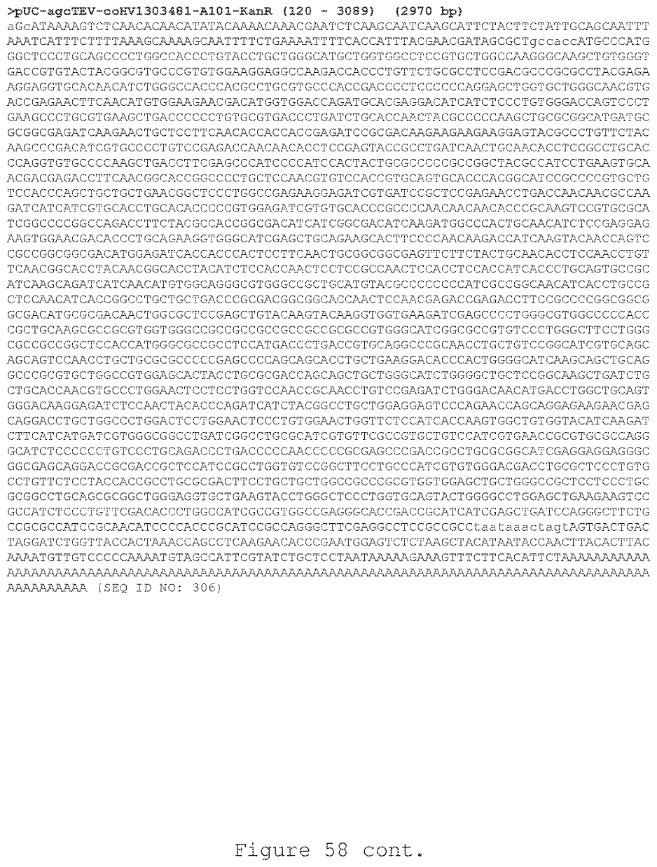

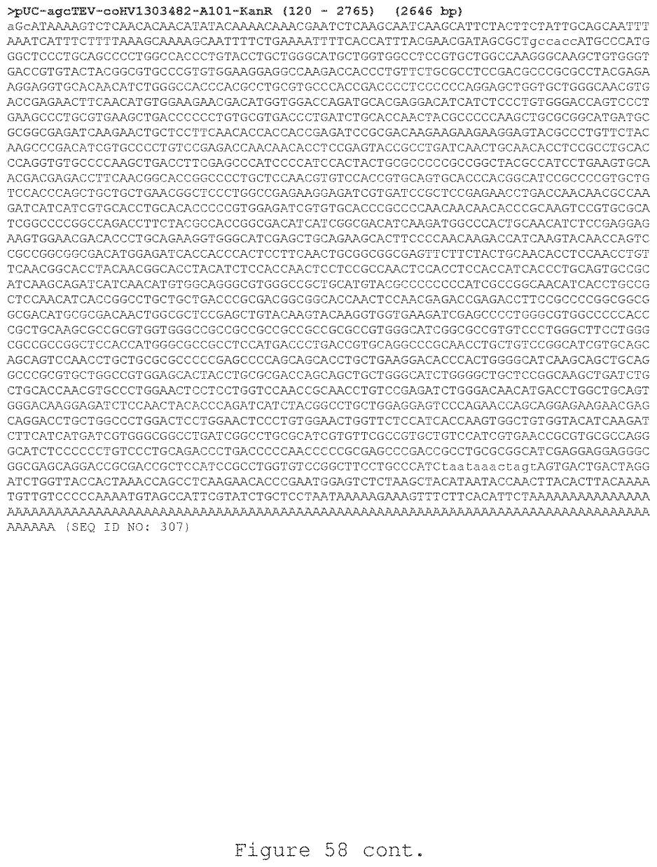

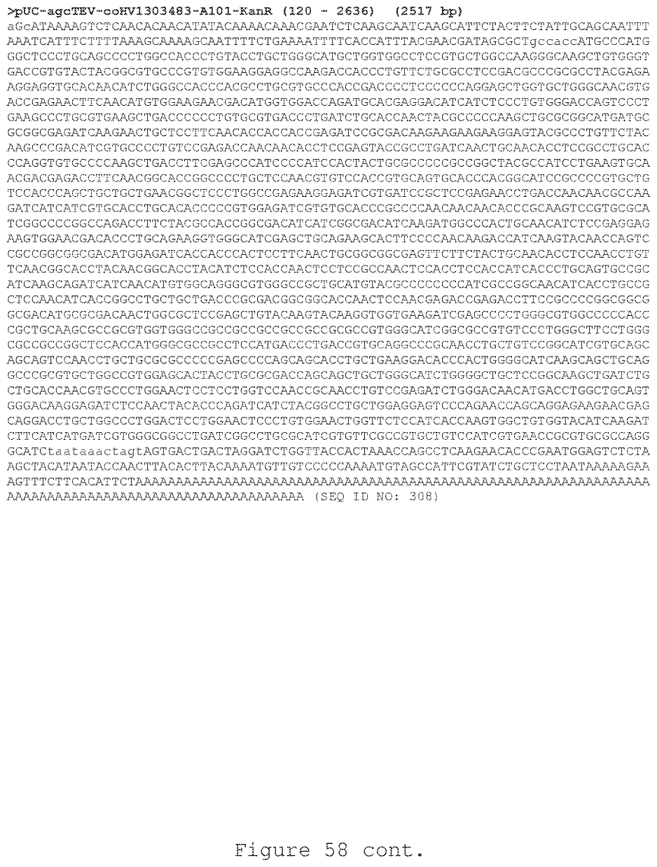

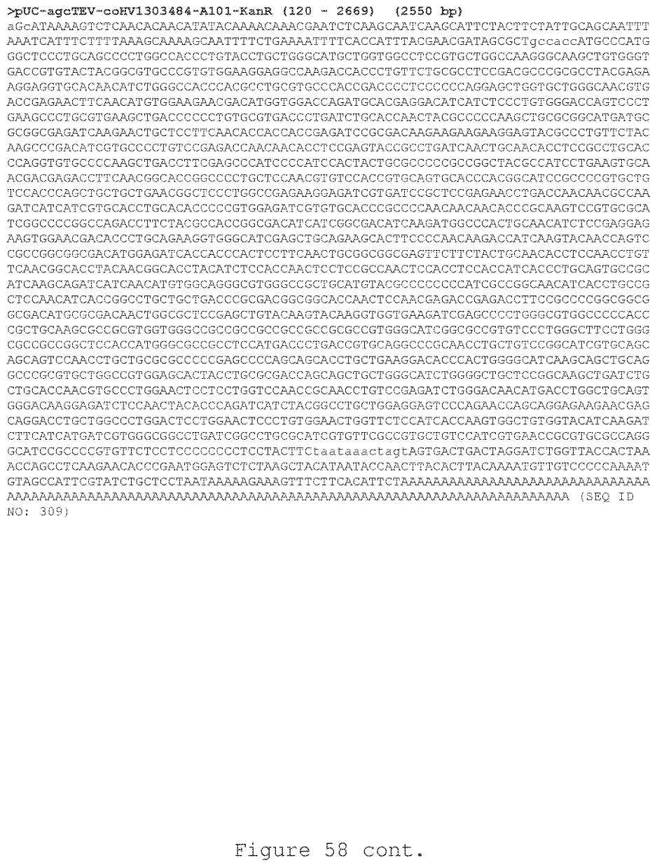

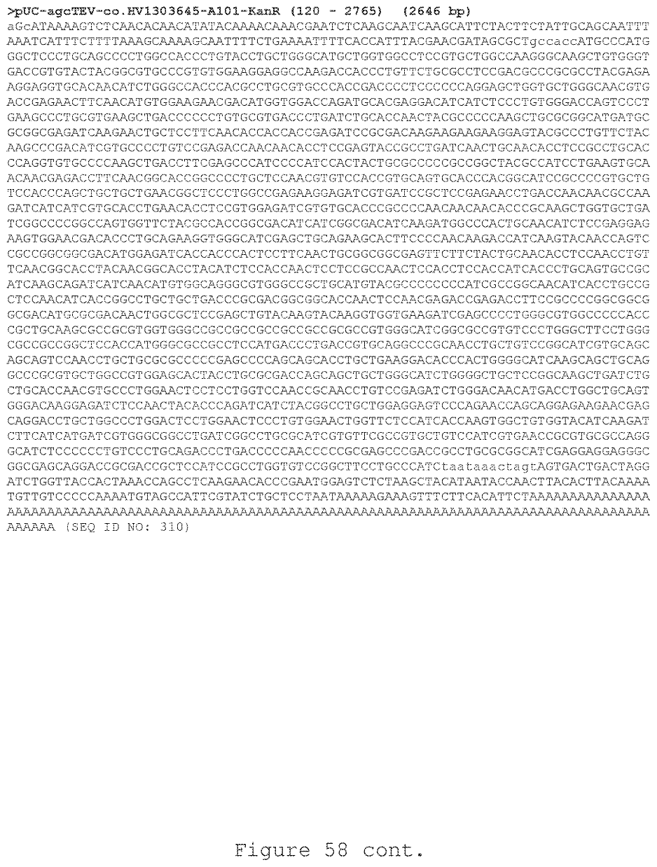

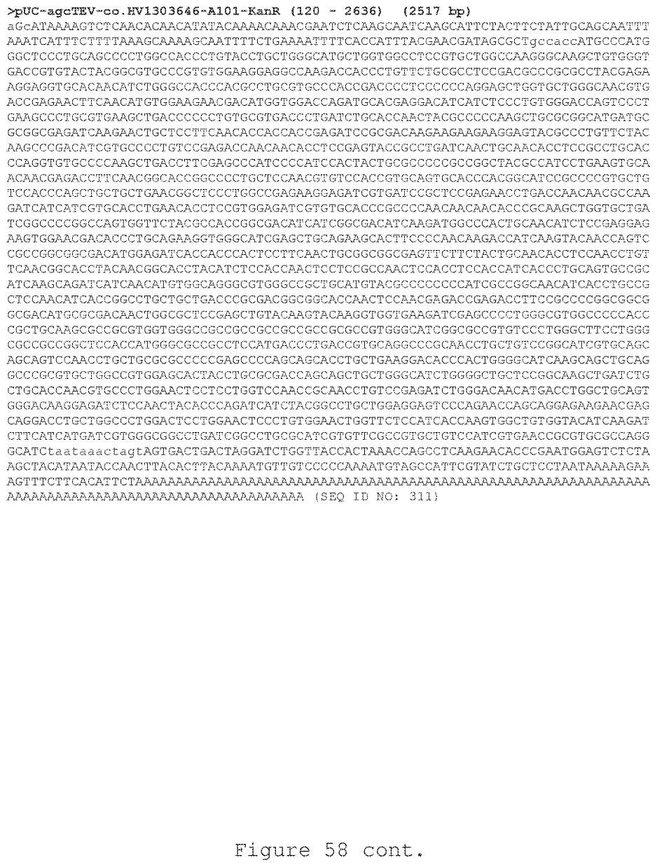

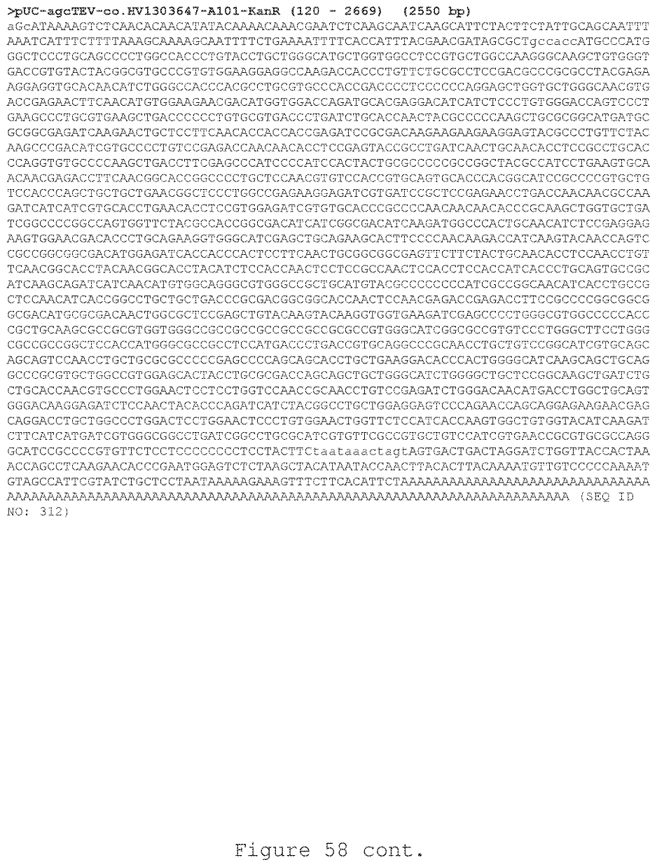

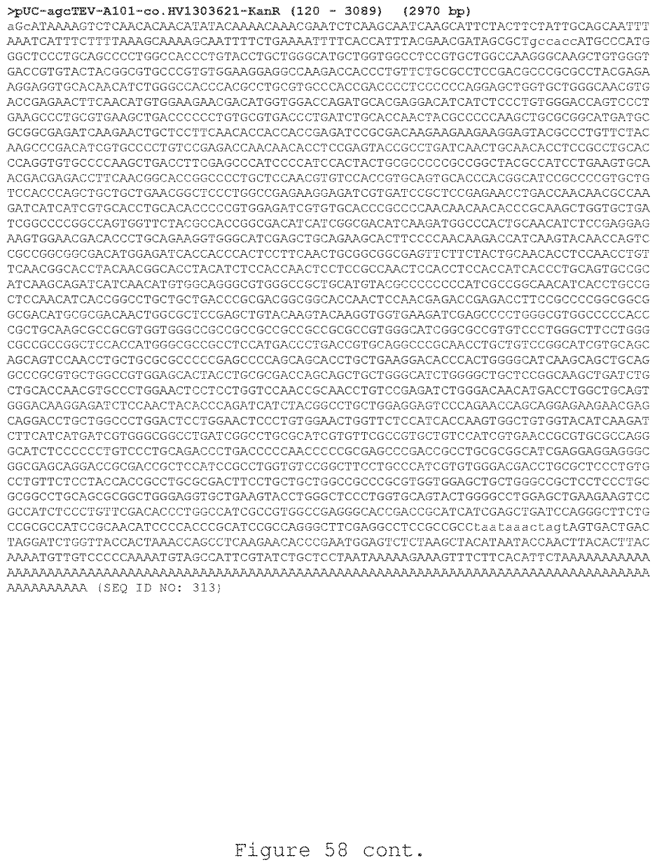

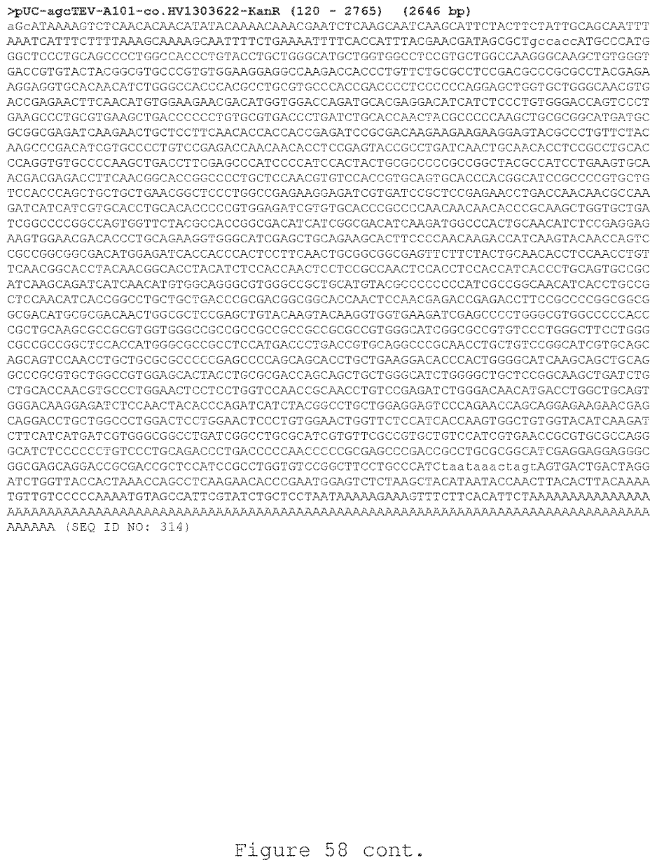

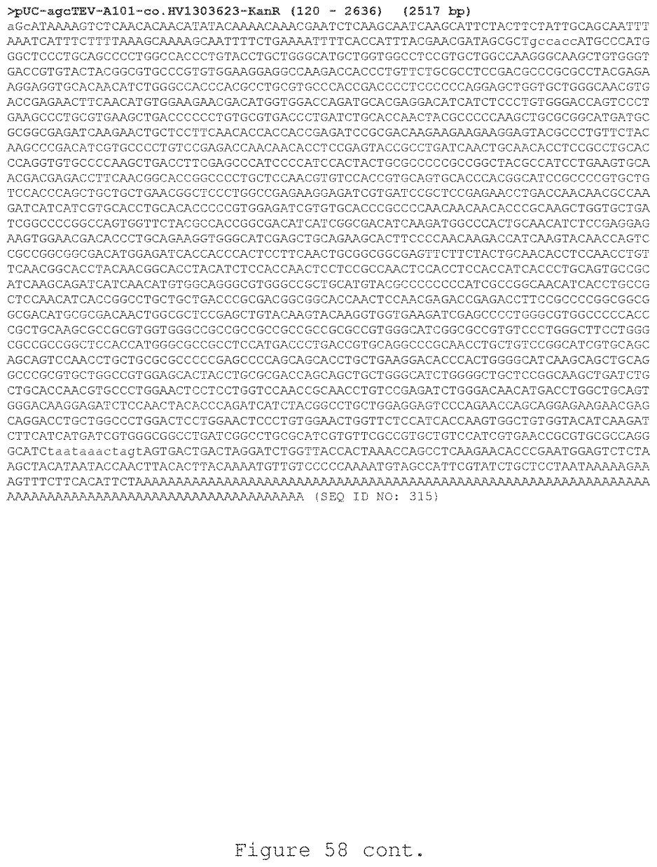

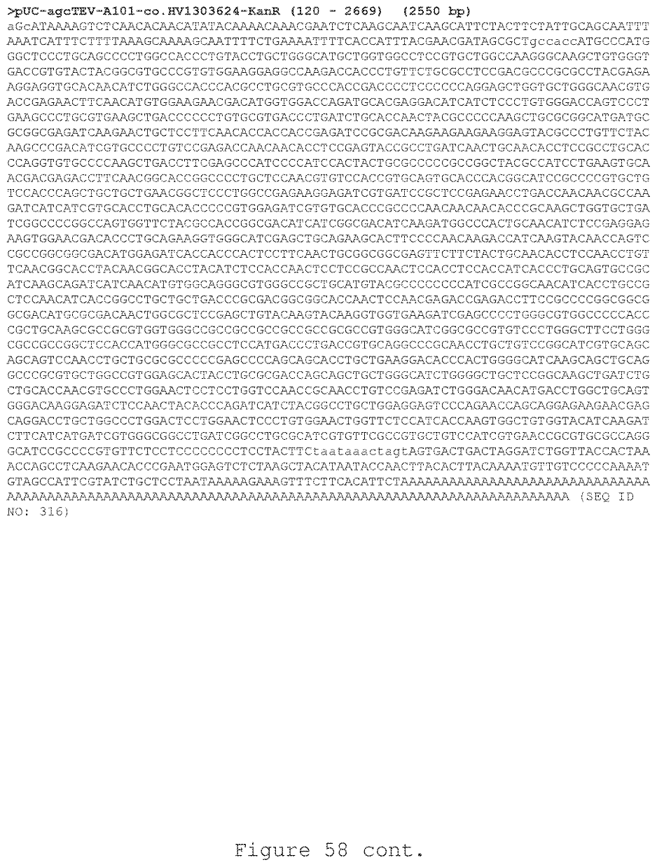

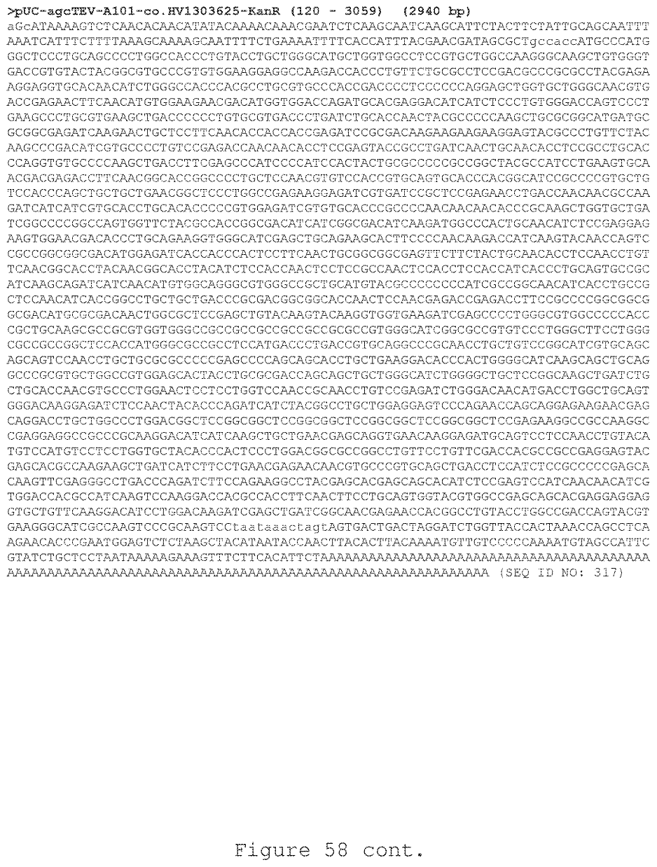

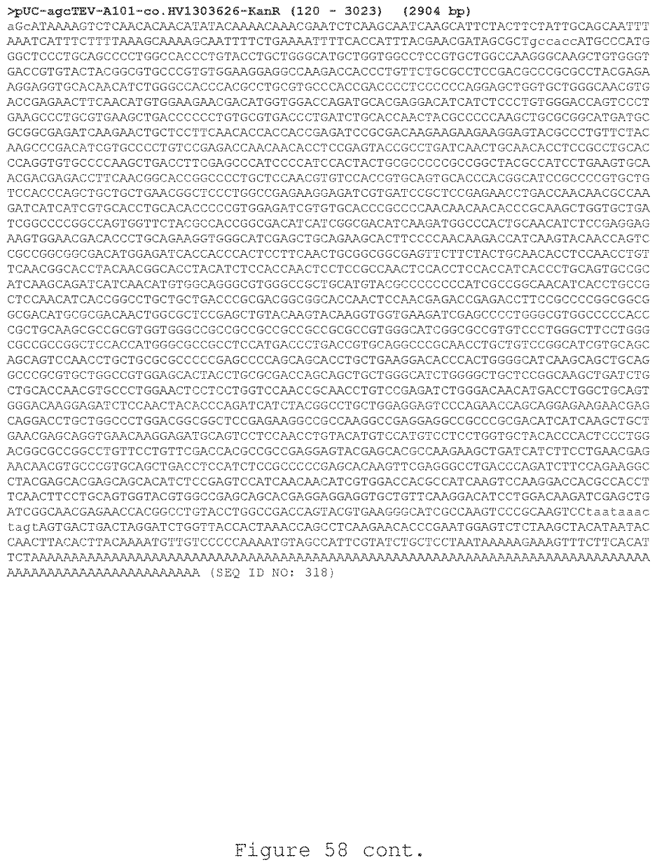

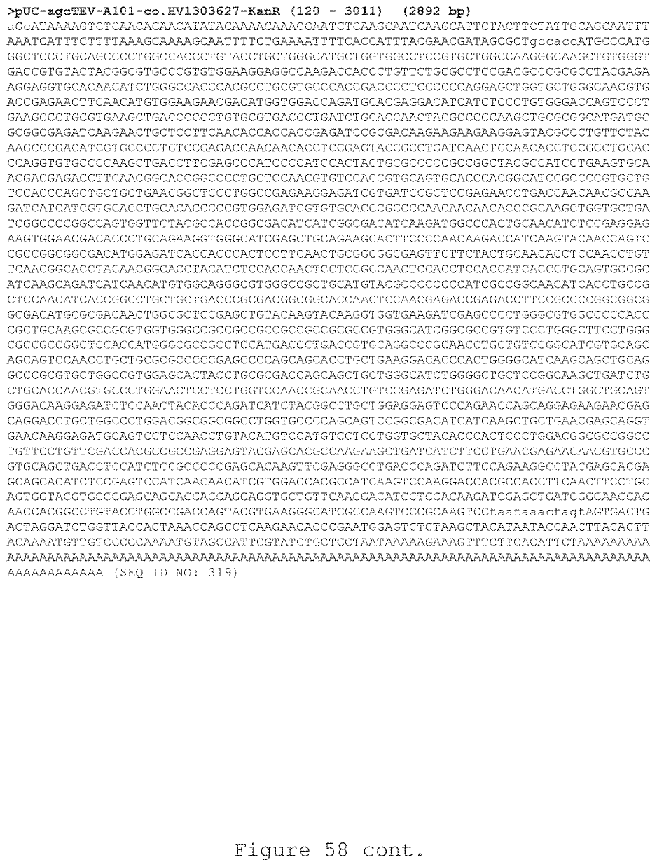

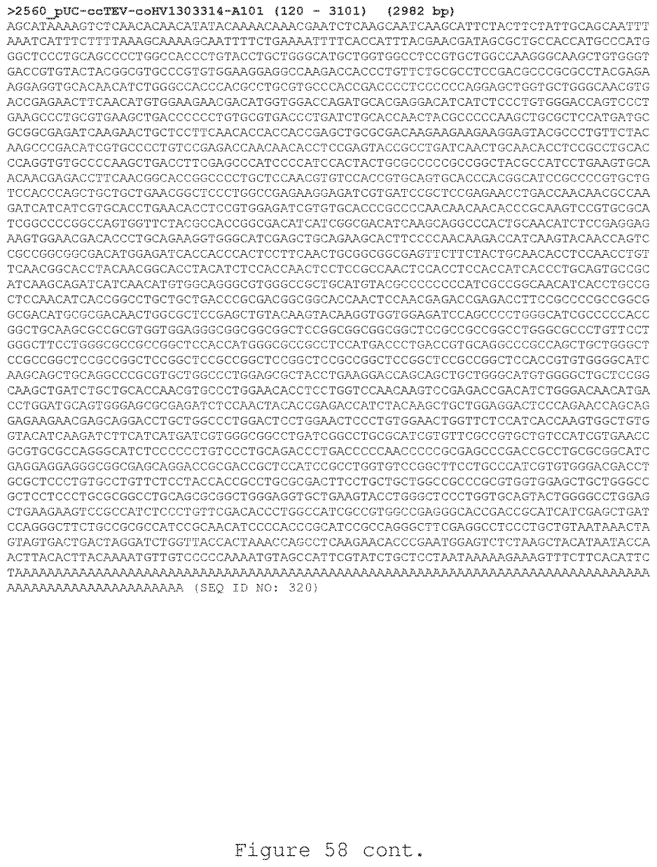

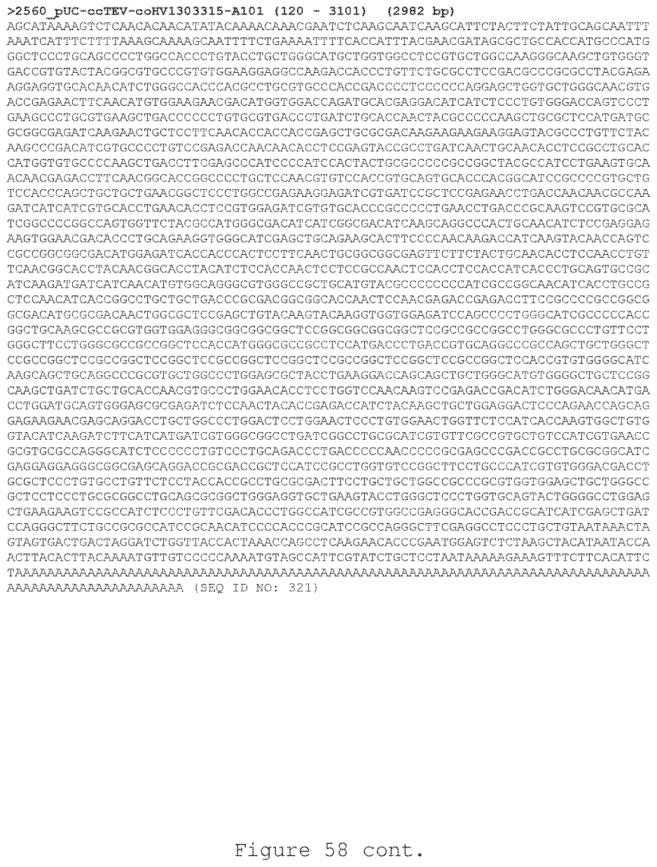

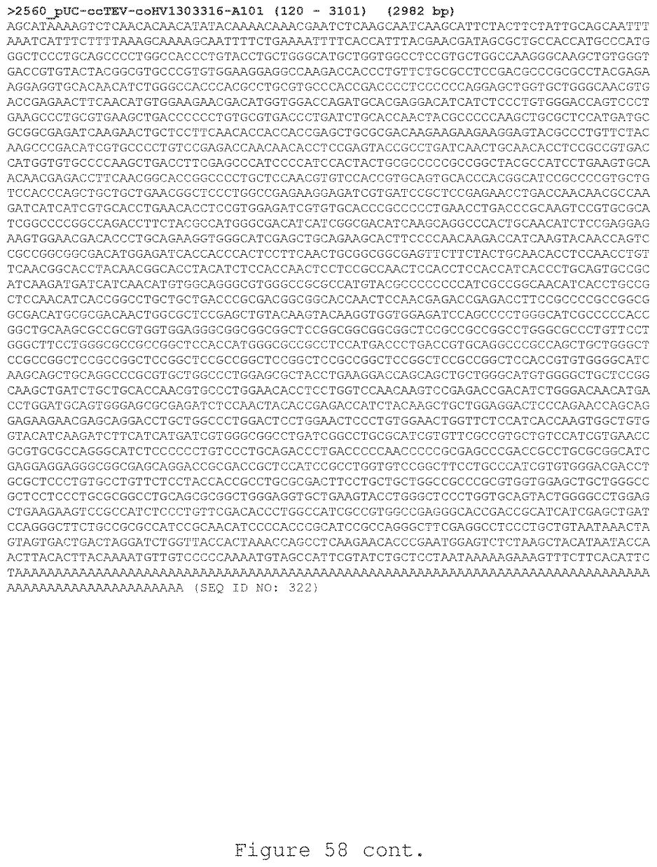

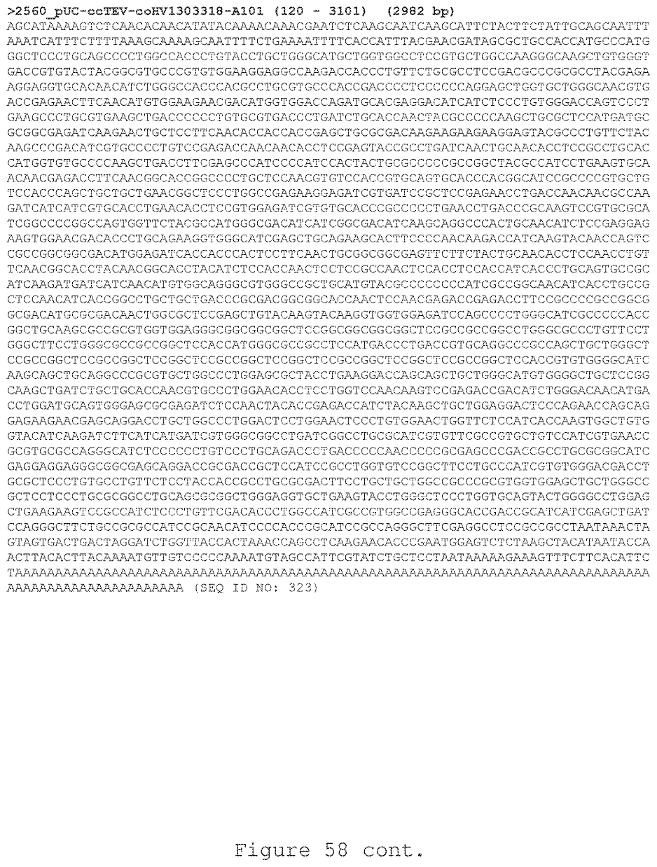

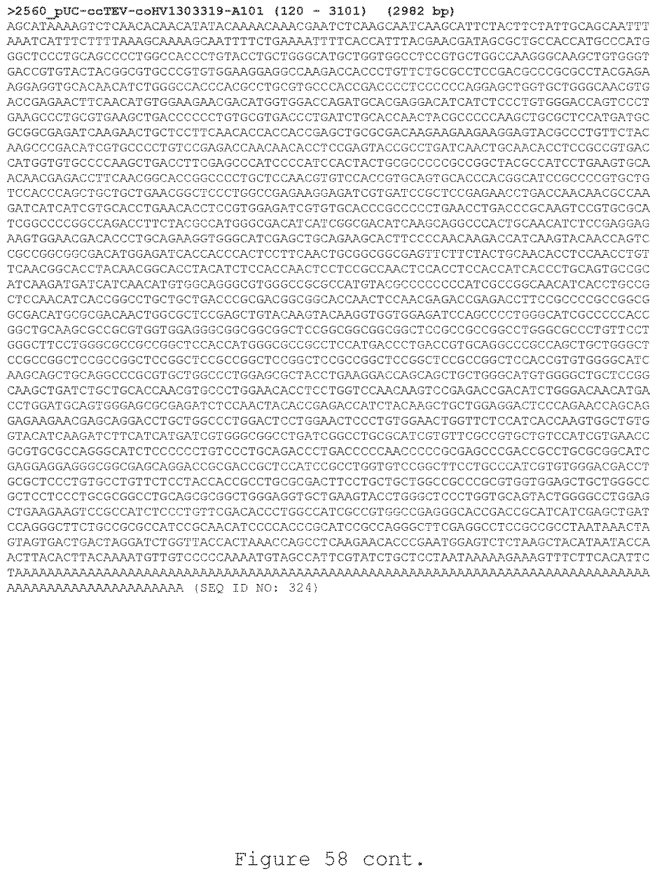

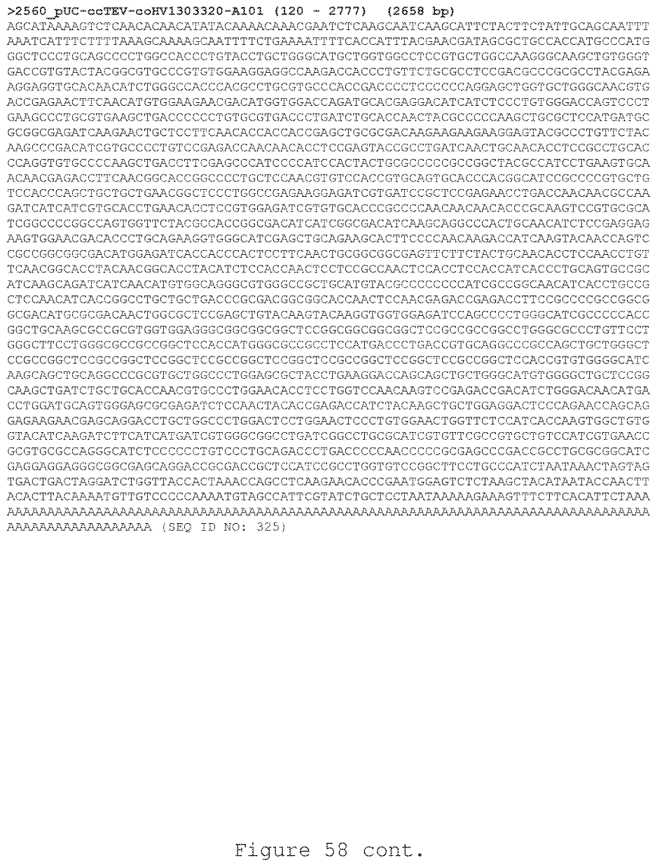

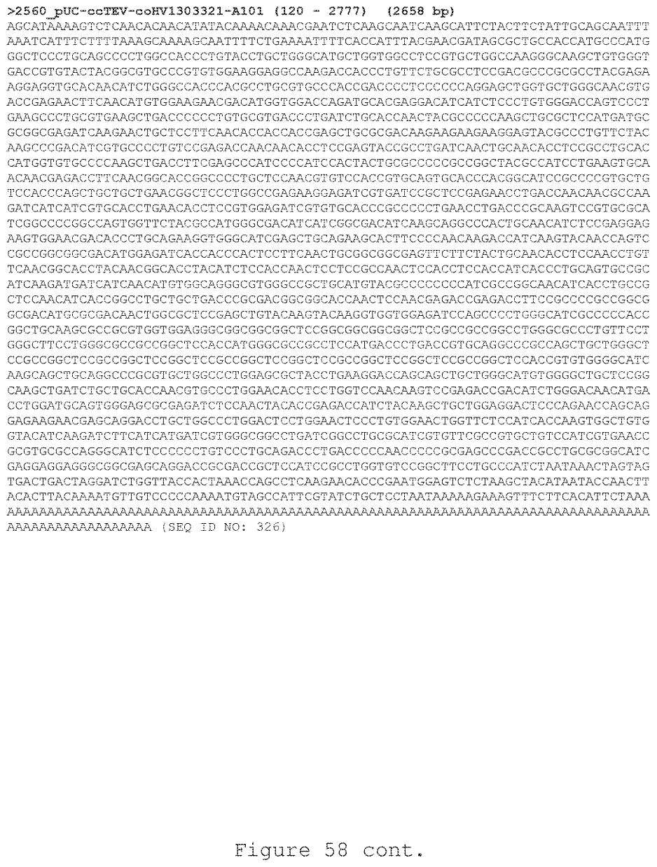

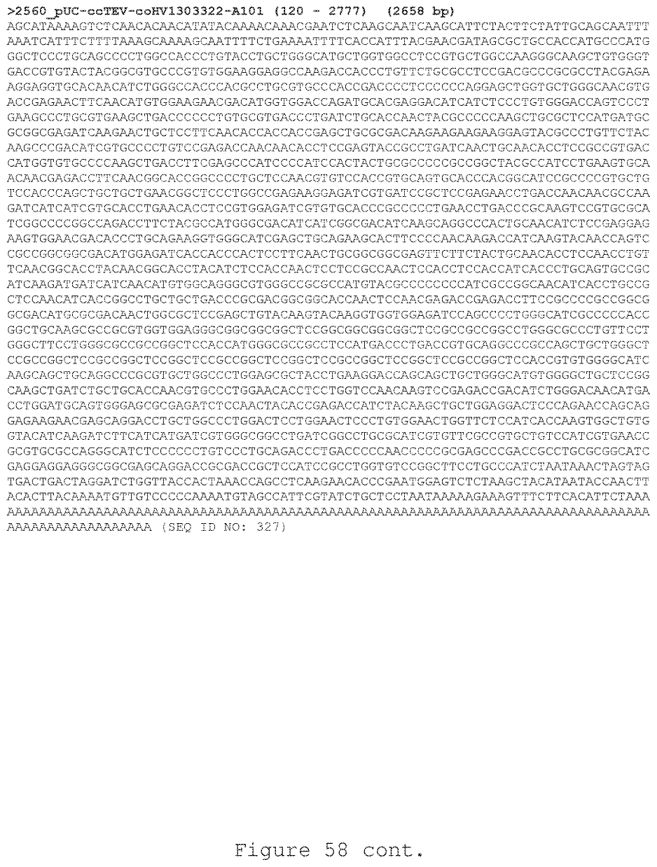

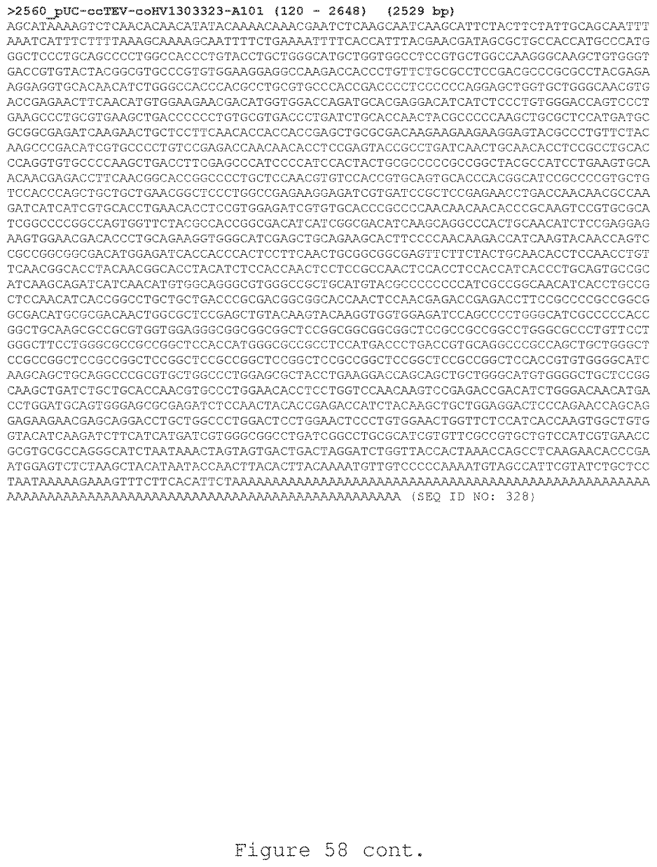

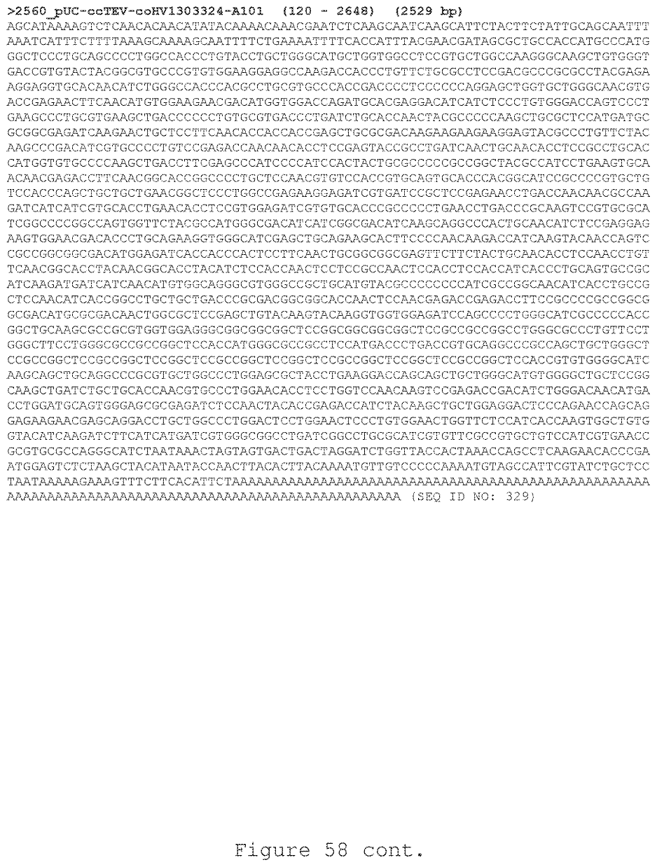

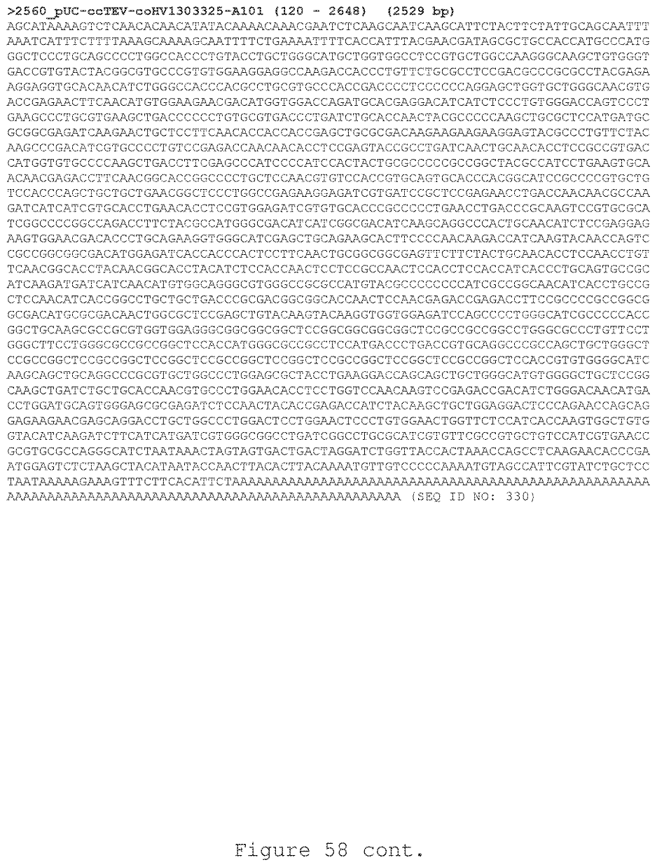

Nucleic acid sequences provided herein, e.g. see FIG. 40 or 58, are provided as DNA sequences. However, it should be understood that such sequences also represent RNA sequences, for example, mRNA. For example, RNA polymerase can be used to make RNA sequences from DNA sequences. In RNA sequences, thymine will be uridine. In some embodiments, uridine will be 1-methyl-pseudouridine. In some embodiments, nucleic acids of the invention, including RNA sequences or mRNAs, can further comprise any type of modified nucleotides, including, but not limited to 5-methyl-cytidine, 6-methyl-adenosine, or modified uridine.

Nucleic acid sequences provided herein, e.g. see FIG. 40 or 58, are provided with a poly A tail length of 101 nucleotides (SEQ ID NO: 10). However, it should be understood that mRNA sequences can comprise different lengths of poly A tail. For example, in some embodiments the poly A tail is about 85 to about 200 nucleotides long. For example, in some embodiments the poly A tail is 85 to 200 nucleotides long (SEQ ID NO: 11). In some embodiments the poly A tail is about 85 to about 110 nucleotides long. In some embodiments the poly A tail is 85 to 110 nucleotides long (SEQ ID NO: 12). In some embodiments the poly A tail is about 90 to about 110 nucleotides long. In some embodiments the poly A tail is 90 to 110 nucleotides long (SEQ ID NO: 13).

In certain aspects the invention provides nucleic acids encoding the inventive envelopes. In non-limiting embodiments, the nucleic acids are mRNA, modified or unmodified, suitable for use any use, for example, but not limited to, use as pharmaceutical compositions. In certain embodiments, the nucleic acids are formulated in lipid, such as but not limited to LNPs.

In some embodiments the immunogens are administered as nucleic acids, including but not limited to mRNAs which could be modified and/or unmodified. See US Pub 20180028645A1, US Pub 20090286852, US Pub 20130111615, US Pub 20130197068, US Pub 20130261172, US Pub 20150038558, US Pub 20160032316, US Pub 20170043037, US Pub 20170327842, U.S. Pat. Nos. 10,006,007, 9,371,511, 9,012,219, US Pub 20180265848, US Pub 20170327842, US Pub 20180344838A1 at least at paragraphs [2060]-[0281], US Pub 20190153425 for non-limiting embodiments of chemical modifications, wherein each content is incorporated by reference in its entirety.

mRNAs delivered in LNP formulations have advantages over non-LNPs formulations. See US Pub 20180028645A1, US Pub 20190274968, US Pub 20180303925, wherein each content is incorporated by reference in its entirety.

In certain embodiments the nucleic acid encoding an envelope is operably linked to a promoter inserted an expression vector. In certain aspects the compositions comprise a suitable carrier. In certain aspects the compositions comprise a suitable adjuvant.

In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects the invention provides nucleic acids comprising any one of the nucleic acid sequences of invention. In certain aspects the invention provides nucleic acids consisting essentially of any one of the nucleic acid sequences of invention. In certain aspects the invention provides nucleic acids consisting of any one of the nucleic acid sequences of invention. In certain embodiments the nucleic acid of the invention, is operably linked to a promoter and is inserted in an expression vector. In certain aspects the invention provides an immunogenic composition comprising the expression vector. In some embodiments, the expression vector comprises DNA.

In certain aspects the invention provides a composition comprising at least one of the nucleic acid sequences of the invention. In certain aspects the invention provides a composition comprising any one of the nucleic acid sequences of invention. In certain aspects the invention provides a composition comprising at least one nucleic acid sequence encoding any one of the polypeptides of the invention.

In one embodiment, the nucleic acid is an RNA molecule. In one embodiment, the RNA molecule is transcribed from a DNA sequence described herein. In some embodiments, the RNA molecule is encoded by one of the inventive sequences. In another embodiment, the nucleotide sequence comprises an RNA sequence transcribed by a DNA sequence encoding any one of the polypeptide sequence of the sequences of the invention, or a variant thereof or a fragment thereof. Accordingly, in one embodiment, the invention provides an RNA molecule encoding one or more of inventive envelopes. The RNA may be plus-stranded. Accordingly, in some embodiments, the RNA molecule can be translated by cells without needing any intervening replication steps such as reverse transcription.

In some embodiments, a RNA molecule of the invention may have a 5′ cap (e.g. but not limited to a 7-methylguanosine, 7 mG(5′)ppp(5′)NImpNp, CleanCap® (e.g., the AG, GG, AU, 3′OMe AG, or 3′OMe GG CleanCap®), or ARCA). This cap can enhance in vivo translation of the RNA. The 5′ nucleotide of an RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. In some embodiments, a RNA molecule useful with the invention may be single-stranded. In some embodiments, a RNA molecule useful with the invention may comprise synthetic RNA.

The recombinant nucleic acid sequence can be an optimized nucleic acid sequence. Such optimization can increase or alter the immunogenicity of the envelope. Optimization can also improve transcription and/or translation. Optimization can include one or more of the following: low GC content leader sequence to increase transcription; mRNA stability and codon optimization; addition of a kozak sequence (e.g., GCC ACC) for increased translation; addition of an immunoglobulin (Ig) leader sequence encoding a signal peptide; and eliminating to the extent possible cis-acting sequence motifs (i.e., internal TATA boxes).

Methods for in vitro transfection of mRNA and detection of envelope expression are known in the art.

Methods for expression and immunogenicity determination of nucleic acid encoded envelopes are known in the art.

In certain aspects the invention contemplates using immunogenic compositions wherein immunogens are delivered as recombinant proteins. Various methods for production and purification of recombinant proteins, including trimers such as, but not limited to, SOSIP based trimers, suitable for use in immunization are known in the art. In certain embodiments recombinant proteins are produced in CHO cells.

It is readily understood that the envelope polypeptides referenced in various examples and figures comprise a signal peptide/leader sequence. It is well known in the art that HIV-1 envelope polypeptide is a secretory protein with a signal peptide or leader sequence that is removed during processing and recombinant expression (without removal of the signal peptide, the protein is not secreted). See for example Li et al. Control of expression, glycosylation, and secretion of HIV-1 gp120 by homologous and heterologous signal sequences. Virology 204 (1): 266-78 (1994) (“Li et al. 1994”), at first paragraph, and Li et al. Effects of inefficient cleavage of the signal sequence of HIV-1 gp120 on its association with calnexin, folding, and intracellular transport. PNAS 93:9606-9611 (1996) (“Li et al. 1996”), at 9609. Any suitable signal peptide sequence could be used. In some embodiments the leader sequence is the endogenous leader sequence. Most of the gp 120 and gp 160 amino acid sequences include the endogenous leader sequence. In other non-limiting examples, the leader sequence is human Tissue Plasminogen Activator (TPA) sequence, human CD5 leader sequence (e.g. MPMGSLQPLATLYLLGMLVASVLA (SEQ ID NO: 19)). Most of the chimeric designs include CD5 leader sequence. A skilled artisan appreciates that when used as immunogens, and for example when recombinantly produced, the amino acid sequences of these proteins do not comprise the signal peptide/leader sequences.

The immunogenic envelopes can also be administered as a protein prime and/or boost alone or in combination with a variety of nucleic acid envelope primes (e.g., HIV-1 Envs delivered as DNA expressed in viral or bacterial vectors).

Dosing of proteins and nucleic acids can be readily determined by a skilled artisan. A single dose of nucleic acid can range from a few nanograms (ng) to hundreds of micrograms (μg) or milligram of a single immunogenic nucleic acid. Recombinant protein dose can range from a few μg micrograms to a few hundred micrograms, or milligrams of a single immunogenic polypeptide.

Administration: The compositions can be formulated in designs that incorporate appropriate carriers such as peptides for enhancing CD4+ T cell help, known as PADRE, GTH1, GTH2, or any combination thereof. In certain embodiments the compositions are delivered via intramuscular (IM), via subcutaneous, via intravenous, via nasal, via mucosal routes, or any other suitable route of immunization.

The compositions can be formulated with appropriate carriers and adjuvants using techniques to yield compositions suitable for immunization. The compositions can include an adjuvant, such as, for example but not limited to 3M052, alum, poly IC, MF-59 or other squalene-based adjuvant, ASOIB, or other liposomal based adjuvant suitable for protein or nucleic acid immunization. In certain embodiments, the adjuvant is GSK AS01E adjuvant containing MPL and QS21. This adjuvant has been shown by GSK to be as potent as the similar adjuvant AS01B but to be less reactogenic using HBsAg as vaccine antigen (Leroux-Roels et al., IABS Conference, April 2013). In certain embodiments, TLR agonists are used as adjuvants. In other embodiment, adjuvants which break immune tolerance are included in the immunogenic compositions.

In certain embodiments, the compositions and methods comprise any suitable agent or immune modulation which could modulate mechanisms of host immune tolerance and release of the induced antibodies. In non-limiting embodiments modulation includes PD-1 blockade; T regulatory cell depletion; CD40L hyperstimulation; soluble antigen administration, wherein the soluble antigen is designed such that the soluble agent eliminates B cells targeting dominant epitopes, or a combination thereof. In certain embodiments, an immunomodulatory agent is administered at a time and in an amount sufficient for transient modulation of the subject's immune response so as to induce an immune response which comprises broad neutralizing antibodies against HIV-1 envelope. Non-limiting examples of such agents is any one of the agents described herein: e.g. chloroquine (CQ), PTPIB Inhibitor-CAS 765317-72-4-Calbiochem or MSI 1436 clodronate or any other bisphosphonate; a Foxol inhibitor, e.g. 344355 Foxol Inhibitor, AS1842856-Calbiochem; Gleevac, anti-CD25 antibody, anti-CCR4 Ab, an agent which binds to a B cell receptor for a dominant HIV-1 envelope epitope, or any combination thereof. In non-limiting embodiments, the modulation includes administering an anti-CTLA4 antibody, OX-40 agonists, or a combination thereof. Non-limiting examples are of CTLA-1 antibody are ipilimumab and tremelimumab. In certain embodiments, the methods comprise administering a second immunomodulatory agent, wherein the second and first immunomodulatory agents are different.

### HIV-1 Envelope Trimers and Other Envelope Designs

Stabilized HIV-1 Env trimer immunogens show enhanced antigenicity for broadly neutralizing antibodies and are not recognized by non-neutralizing antibodies. Described herein are additional envelope modifications and designs. In some embodiments these envelopes, including but not limited to trimers are further multimerized, and/or used as particulate, high-density array in liposomes or other particles, for example but not limited to nanoparticles. Any one of the envelopes of the invention could be designed and expressed as described herein.

A stabilized chimeric SOSIP designs can be used to generate HIV-1 envelope trimers. This design is applicable to diverse viruses from multiple clades. SOSIP designs can be applied to any CH848 envelopes comprising residues 132-154 (HXB2 numbering) of the N332-GT5 V1 loop disclosed herein including those in Table 5 and Table 6.

Elicitation of neutralizing antibodies is one goal for antibody-based vaccines. Neutralizing antibodies target the native trimeric HIV-1 Env on the surface virions. The trimeric HIV-1 envelope protein consists of three protomers each containing a gp 120 and gp41 heterodimer. Recent immunogen design efforts have generated soluble near-native mimics of the Env trimer that bind to neutralizing antibodies but not non-neutralizing antibodies. The recapitulation of the native trimer could be a key component of vaccine induction of neutralizing antibodies. Neutralizing Abs target the native trimeric HIV-1 Env on the surface of viruses (Poignard et al. J Virol. 2003 January;77 (1): 353-65; Parren et al. J Virol. 1998 December;72 (12): 10270-4.; Yang et al. J Virol. 2006 November;80 (22): 11404-8.). The HIV-1 Env protein consists of three protomers of gp120 and gp41 heterodimers that are noncovalently linked together (Center et al. J Virol. 2002 August;76 (15): 7863-7.). Soluble near-native trimers preferentially bind neutralizing antibodies as opposed to non-neutralizing antibodies (Sanders et al. PLOS Pathog. 2013 September; 9 (9): e1003618).

Provided here are non-limiting embodiments of well-folded trimers for Env immunizations. Provided are engineered trimeric immunogens derived from CH848 viruses. These immunogens were designed as chimeric proteins that possess the BG505 gp41 connected to the CH848 gp120, since the BGS05 strain is particularly adept at forming well-folded, closed trimers. This envelope design retains the portion of CH848 that is targeted by the V3 glycan broadly neutralizing antibody lineages.

Recombinant envelopes as trimers could be produced and purified by any suitable method. For a non-limiting example of purification methods see Ringe R P, Yasmeen A, Ozorowski G, Go E P, Pritchard L K, Guttman M, Ketas T A, Cottrell C A, Wilson I A, Sanders R W, Cupo A, Crispin M, Lee K K, Desaire H, Ward A B, Klasse P J, Moore J P. 2015. Influences on the design and purification of soluble, recombinant native-like HIV-1 envelope glycoprotein trimers. J Virol 89:12189-12210. doi: 10.1128/JVI.01768-15.

### Multimeric Envelopes

Presentation of antigens as particulates reduces the B cell receptor affinity necessary for signal transduction and expansion (see Baptista et al. EMBO J. 2000 Feb. 15; 19 (4): 513-520). Displaying multiple copies of the antigen on a particle provides an avidity effect that can overcome the low affinity between the antigen and B cell receptor. The initial B cell receptor specific for pathogens can be low affinity, which precludes vaccines from being able to stimulate and expand B cells of interest. In particular, very few naïve B cells from which HIV-1 broadly neutralizing antibodies arise can bind to soluble HIV-1 Envelope. Provided are envelopes, including but not limited to trimers as particulate, high-density array on liposomes or other particles, for example but not limited to nanoparticles. See, e.g. He et al. Nature Communications 7, Article number: 12041 (2016), doi: 10.1038/ncomms12041; Bamrungsap et al. Nanomedicine, 2012, 7 (8), 1253-1271.

Multimeric nanoparticles that comprise and/or display HIV envelope protein or fragments on their surface can be used a vaccine immunogens.

In some instances, the nucleic acid encoding an antigen (e.g., an HIV-envelope polypeptide) is fused via a linker/spacer to a nucleic acids sequence encoding a protein which can self-assemble. Upon translation, a fusion protein is made that can self-assemble into a multimeric complex—also referred to as a nanoparticle displaying multiple copies of the antigen. In other instances, the protein antigen could be conjugated to the self-assembling protein via an enzymatic reaction, thereby forming a nanoparticle displaying multiple copies of the antigen. Non-limiting embodiments of enzymatic conjugation include without limitation sortase mediated conjugation. In some embodiments, linkers for use in any of the designs of the invention could be 2-50 amino acids long, e.g. 2, 3, 4, 5, 6, 7, 8, 9, 10, 11, 12, 13, 14, 15, 16, 17, 18, 19, 20, 25, 30, 35, 40, 45, or 50 amino acids long. In certain embodiments, these linkers comprise glycine and serine amino acid in any suitable combination, and/or repeating units of combinations of glycine, serine and/or alanine.

Ferritin is a well-known protein that self-assembles into a hollow particle composed of repeating subunits. In some species ferritin nanoparticles are composed of 24 copies of a single subunit, whereas in other species it is composed of 12 copies each of two subunits.

To improve the interaction between the naïve B cell receptor and immunogens, in some embodiments, an envelope design is created so the envelope is presented on particles, e.g. but not limited to nanoparticle. In some embodiments, the HIV-1 envelope trimer could be fused to ferritin. Ferritin protein self assembles into a small nanoparticle with three-fold axis of symmetry. At these axes the envelope protein is fused. Therefore, the assembly of the three-fold axis also clusters three HIV-1 envelope protomers together to form an envelope trimer. Each ferritin particle has 8 axes which equates to 8 trimers being displayed per particle. See e.g. Sliepen et al. Retrovirology 2015 12:82, DOI: 10.1186/s12977-015-0210-4.

Any suitable ferritin sequence could be used. In non-limiting embodiments, ferritin sequences are disclosed in U.S. Pat. No. 10,961,283, the content of which is hereby incorporated by reference in its entirety.

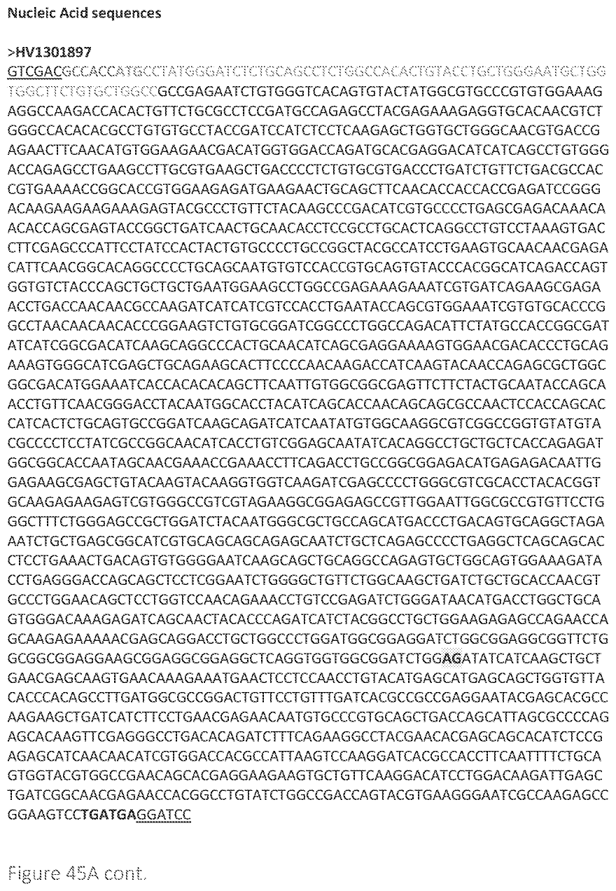

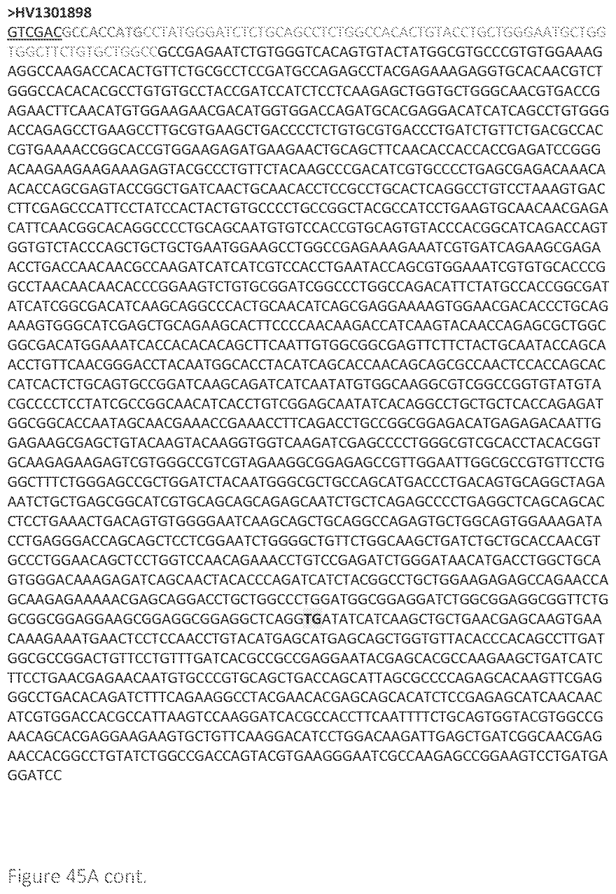

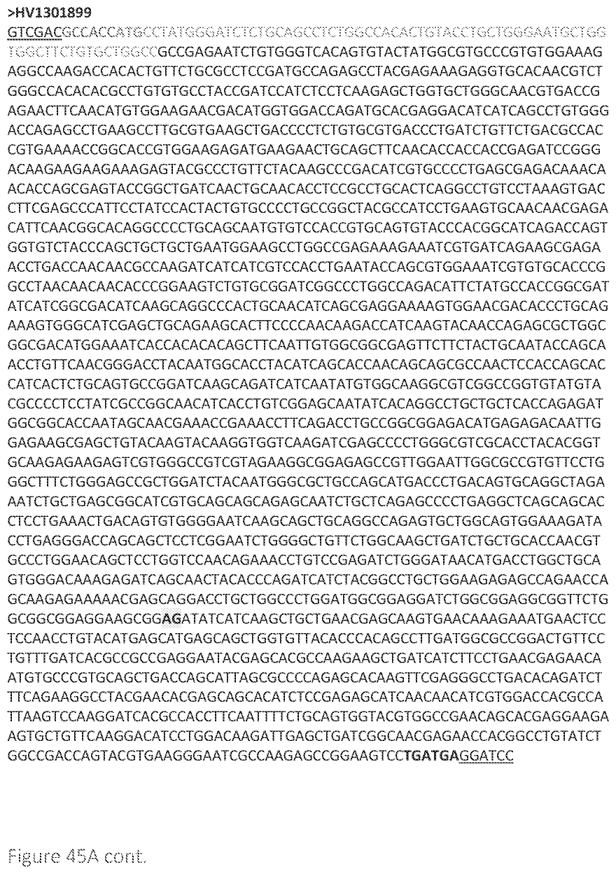

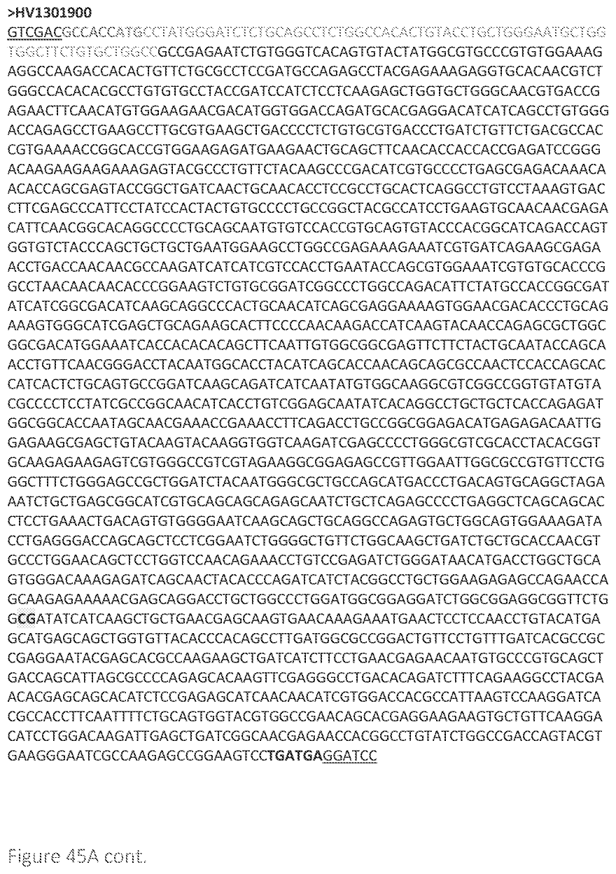

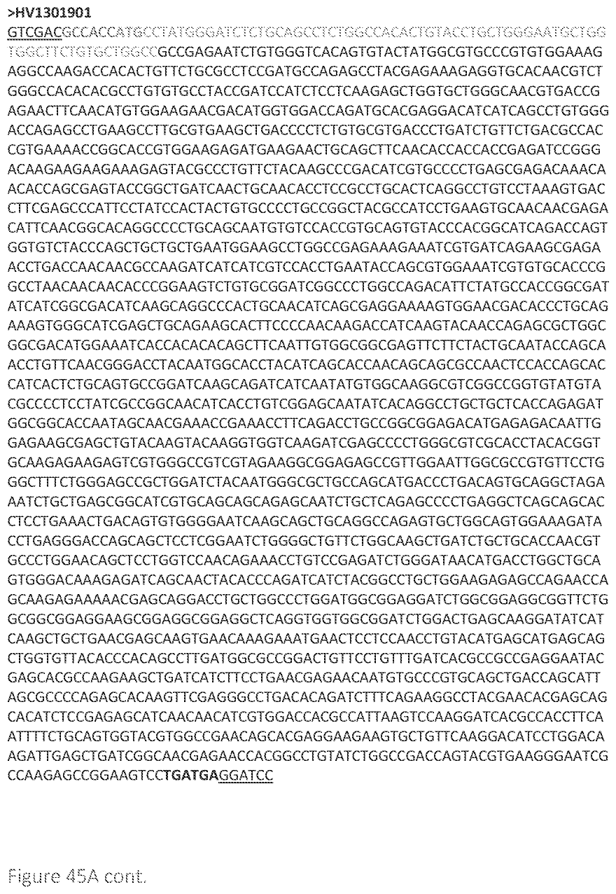

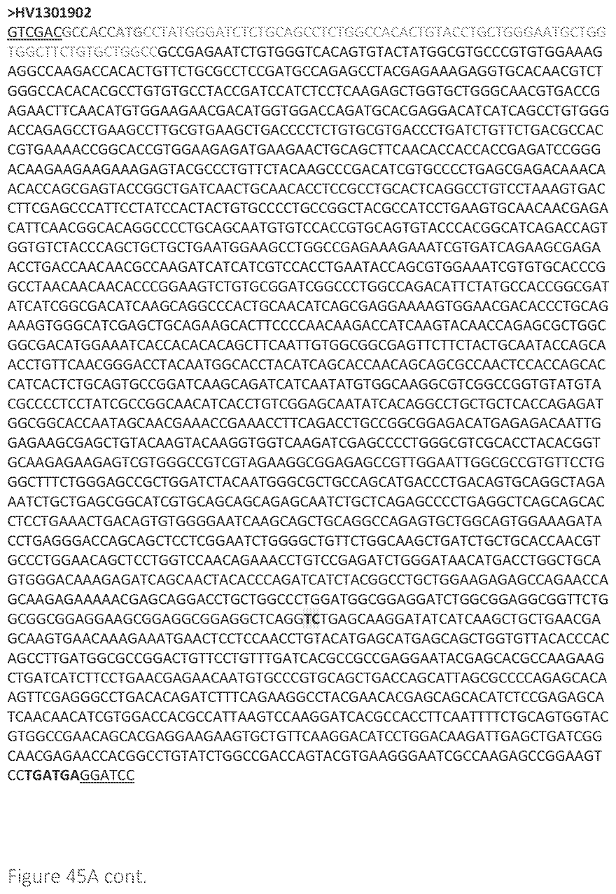

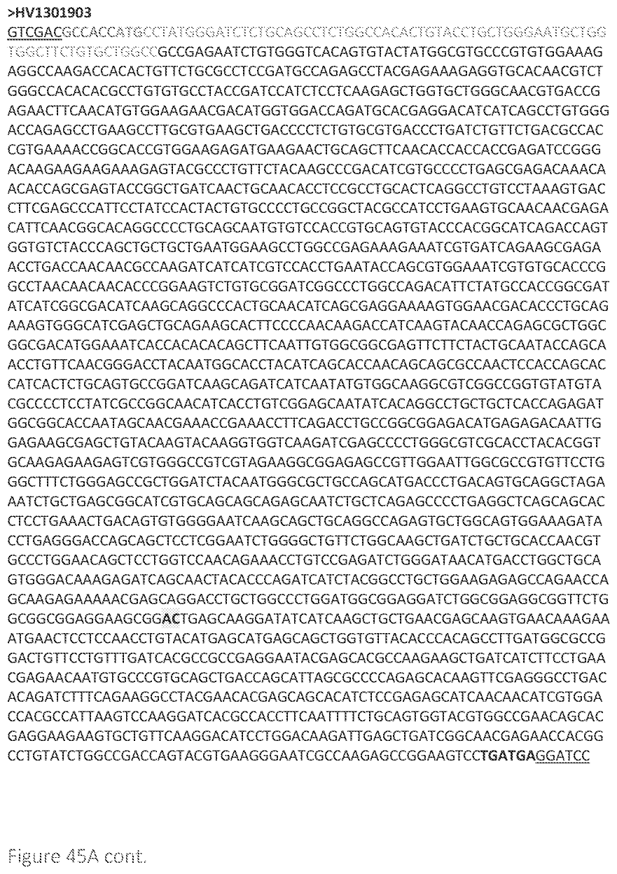

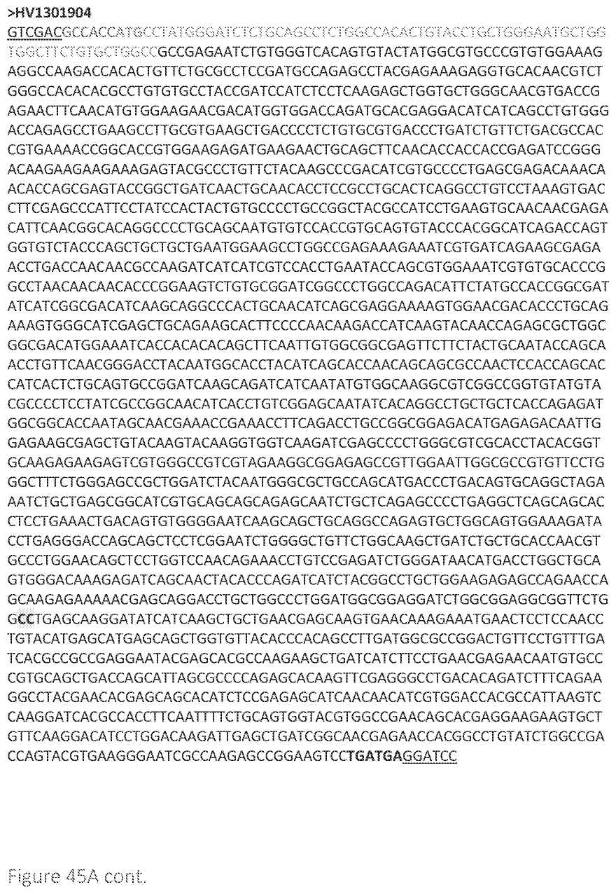

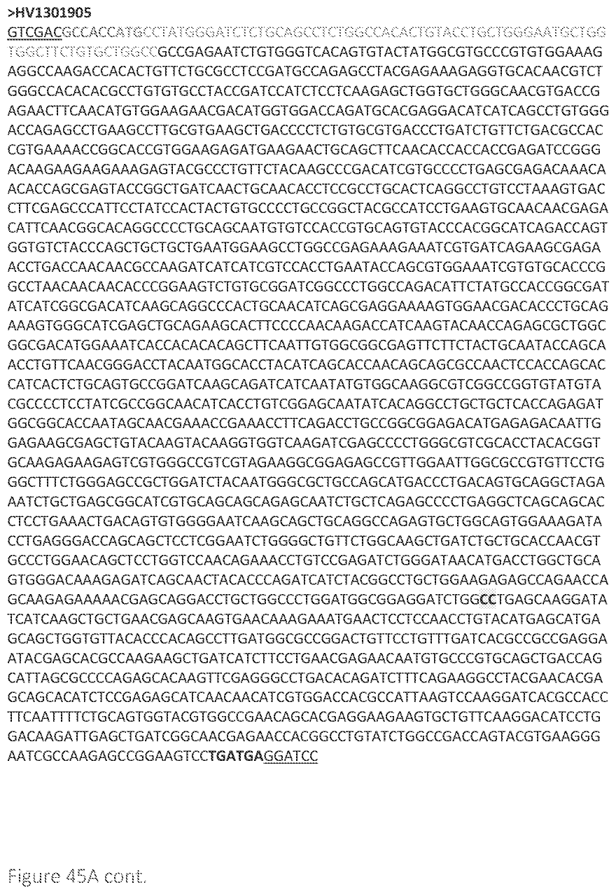

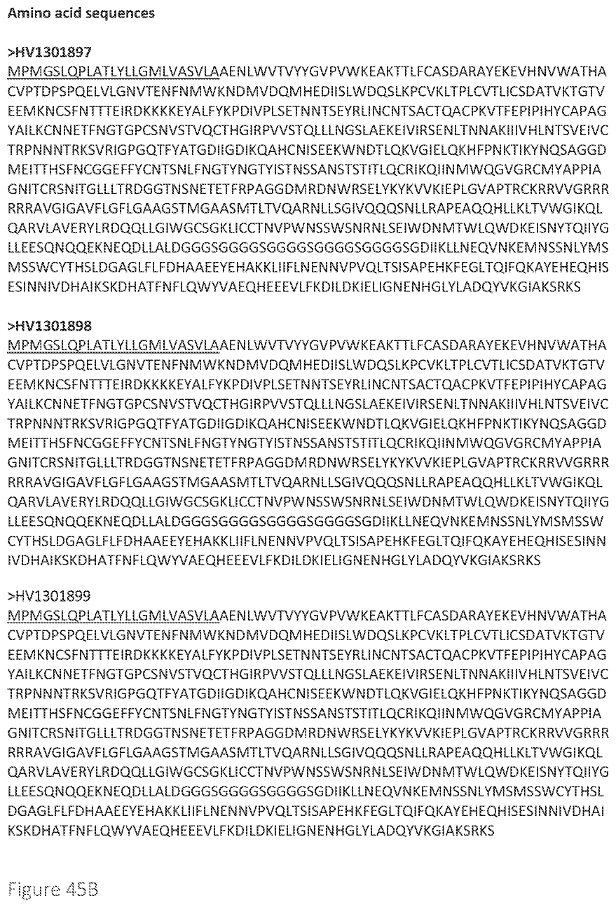

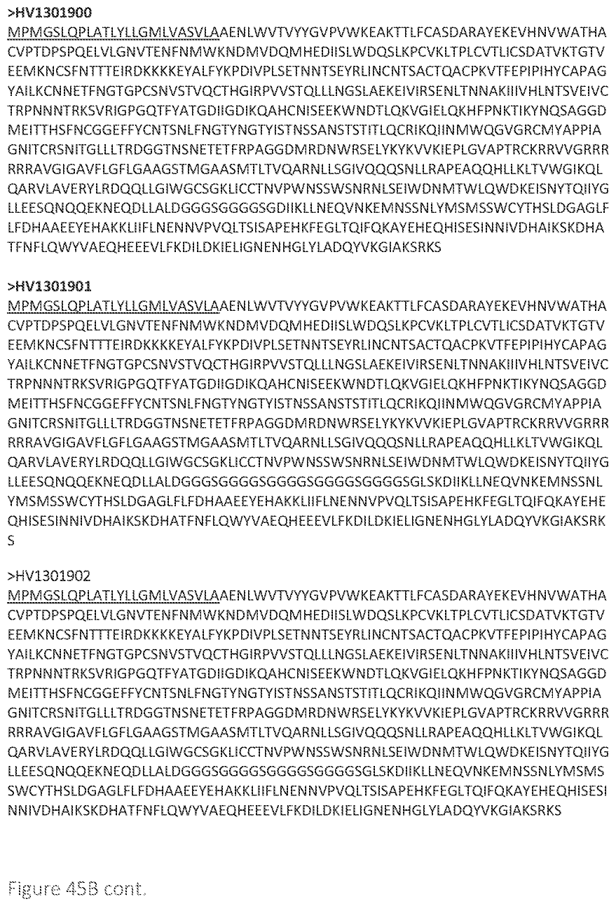

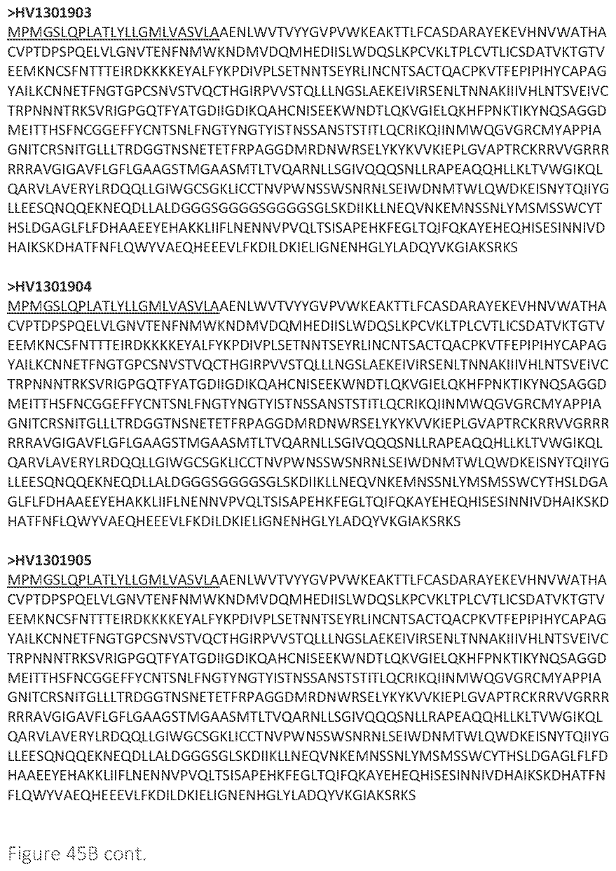

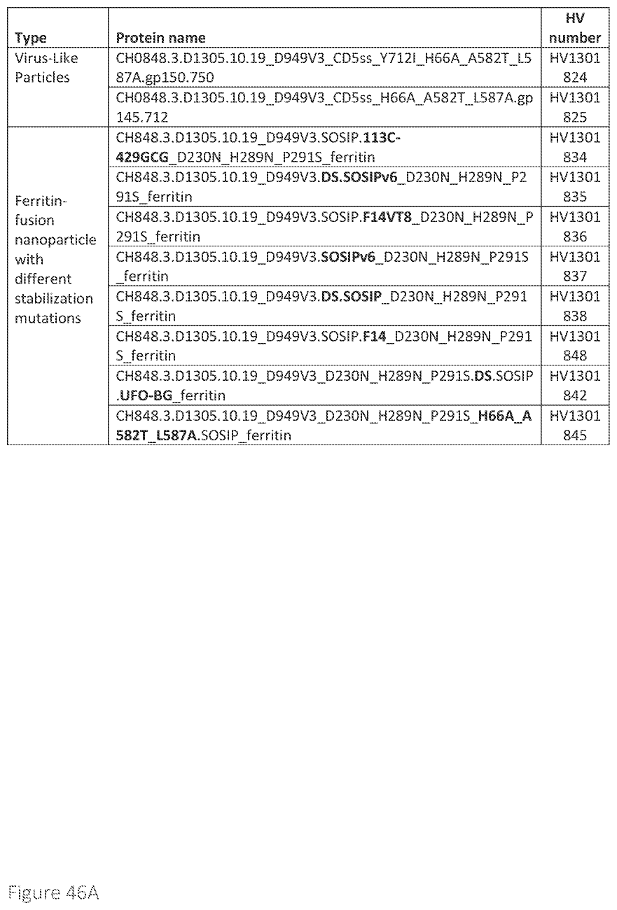

Ferritin nanoparticle linkers: The ability to form HIV-1 envelope ferritin nanoparticles relies self-assembly of 24 ferritin subunits into a single ferritin nanoparticle. The addition of a ferritin subunit to the C-terminus of HIV-1 envelope may interfere with the ability of the ferritin subunit to fold properly and or associate with other ferritin subunits. When expressed alone ferritin readily forms 24-subunit nanoparticles, however appending it to envelope only yields nanoparticles for certain envelopes. Since the ferritin nanoparticle forms in the absence of envelope, the envelope could be sterically hindering the association of ferritin subunits. Thus, ferritin can be designed with elongated glycine-serine linkers to further distance the envelope from the ferritin subunit. To make sure that the glycine linker is attached to ferritin at the correct position, constructs can be created that attach at second amino acid position or the fifth amino acid position. The first four n-terminal amino acids of natural Helicobacter pylori ferritin are not needed for nanoparticle formation but may be critical for proper folding and oligomerization when appended to envelope. Thus, constructs can be designed with and without the leucine, serine, and lysine amino acids following the glycine-serine linker. The goal will be to find a linker length that is suitable for formation of envelope nanoparticles when ferritin is appended to most envelopes. For non-limiting embodiments, linker designs see FIGS. 45A-B. Any suitable linker between the envelope and ferritin could be used, so long as the fusion protein is expressed and the trimer is formed.

The nanoparticle immunogens are composed of various forms of HIV-1 envelope protein, e.g. without limitation envelope trimer, and self-assembling protein, e.g. without limitation ferritin protein. Any suitable ferritin could be used in the immunogens of the invention. In non-limiting embodiments, the ferritin is derived from Helicobacter pylori. In non-limiting embodiments, the ferritin is insect ferritin. In non-limiting embodiments, each nanoparticle displays 24 copies of the envelope protein on its surface.

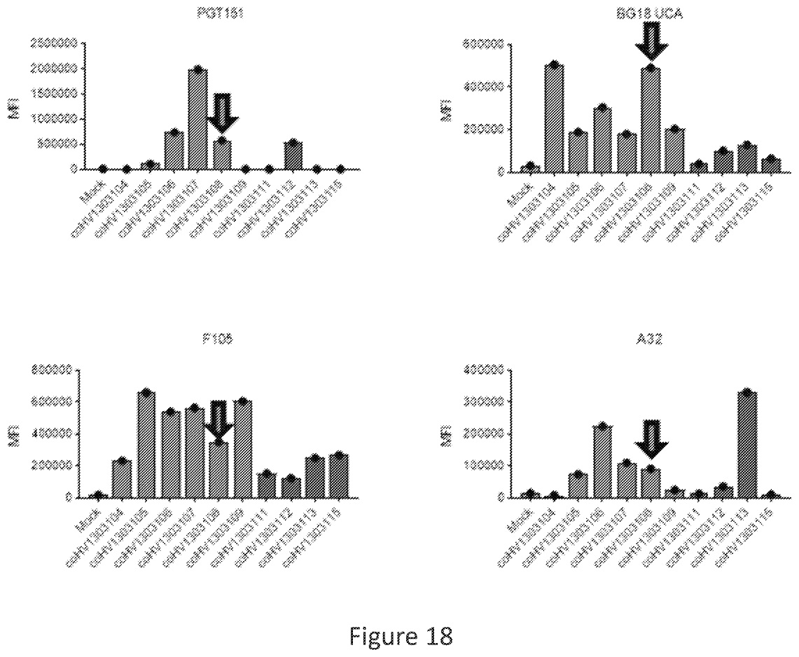

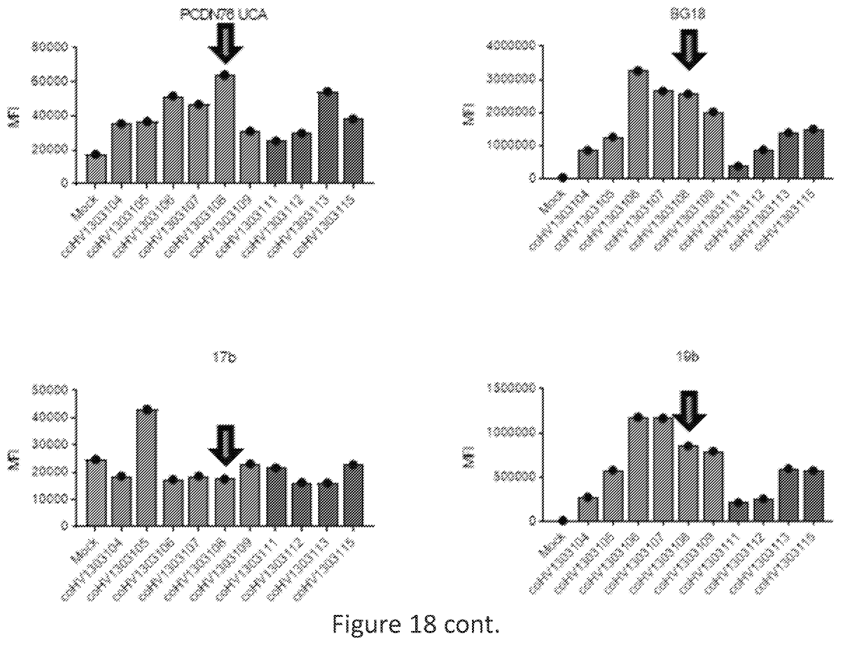

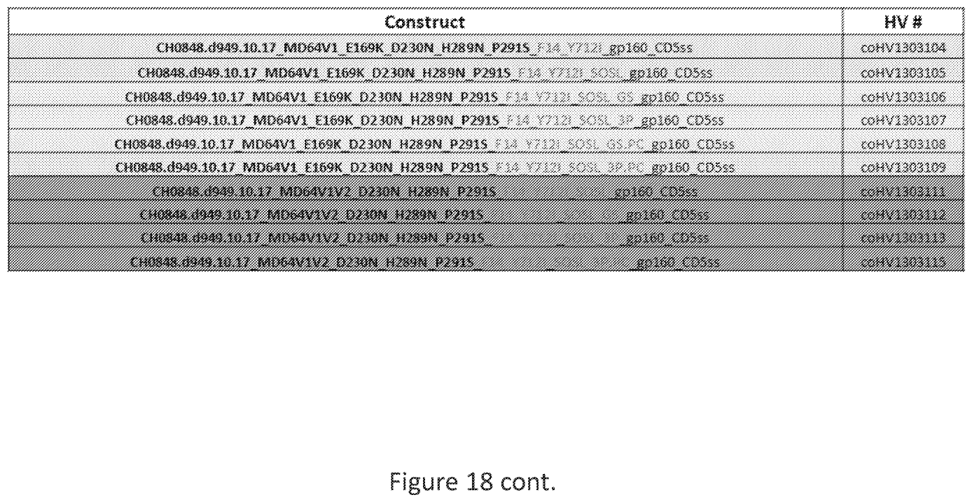

Another approach to multimerize expression constructs uses staphylococcus sortase A transpeptidase ligation to conjugate inventive envelope trimers, for example but not limited to cholesterol. The trimers can then be embedded into liposomes via the conjugated cholesterol. To conjugate the trimer to cholesterol either a C-terminal LPXTG tag (SEQ ID NO: 14) or a N-terminal pentaglycine repeat tag (SEQ ID NO: 18) is added to the envelope trimer gene. Cholesterol is also synthesized with these two tags. In a non-limiting embodiment, a C-terminal tag is LPXTGG, where X signifies any amino acid but most commonly Ala, Ser, Glu (SEQ ID NO: 15). Sortase A is then used to covalently bond the tagged envelope to the cholesterol. The sortase A-tagged trimer protein can also be used to conjugate the trimer to other peptides, proteins, or fluorescent labels. In non-limiting embodiments, the sortase A tagged trimers are conjugated to ferritin to form nanoparticles. See FIG. 18.

The invention provides design of envelopes and trimer designs wherein the envelope comprises a linker which permits addition of a lipid, such as but not limited to cholesterol, via a sortase A reaction. See e.g. Tsukiji, S. and Nagamune, T. (2009), Sortase-Mediated Ligation: A Gift from Gram-Positive Bacteria to Protein Engineering. ChemBioChem, 10:787-798. doi: 10.1002/cbic.200800724; Proft, T. Sortase-mediated protein ligation: an emerging biotechnology tool for protein modification and immobilisation. Biotechnol Lett (2010) 32:1. doi: 10.1007/s10529-009-0116-0; Lena Schmohl, Dirk Schwarzer, Sortase-mediated ligations for the site-specific modification of proteins, Current Opinion in Chemical Biology, Volume 22, October 2014, Pages 122-128, ISSN 1367-5931, dx.doi.org/10.1016/j.cbpa.2014.09.020; Tabata et al. Anticancer Res. 2015 August;35 (8): 4411-7; Pritz et al. J. Org. Chem. 2007, 72, 3909-3912.

The lipid modified envelopes and trimers could be formulated as liposomes. Any suitable liposome composition is contemplated.

The lipid modified and multimerized envelopes and trimers could be formulated as liposomes. Any suitable liposome composition is contemplated.

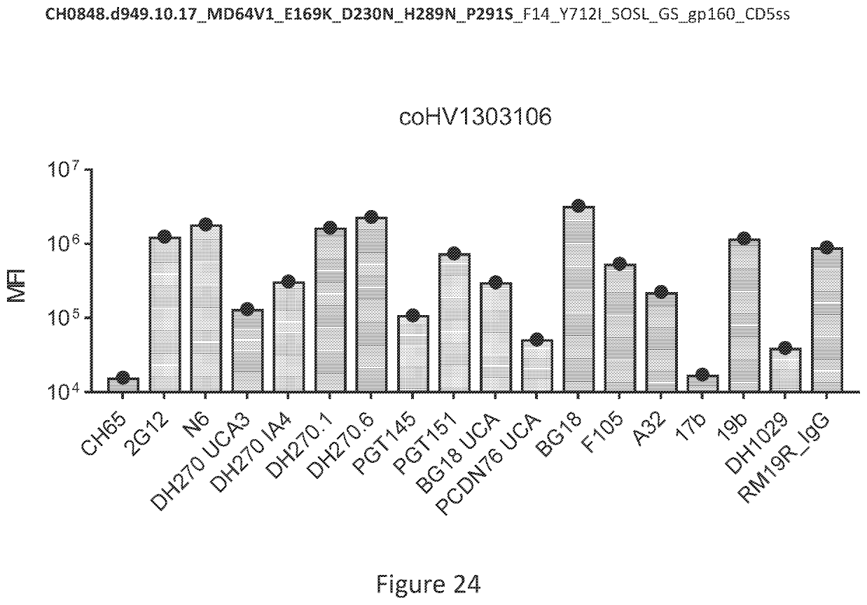

Non-limiting embodiments of envelope designs for use in sortase A reaction are shown in FIG. 24B-D of US2020/0002383, incorporated by reference in its entirety.

Additional sortase linkers could be used so long as their position allows multimerization of the envelopes. In a non-limiting embodiment, a C-terminal tag is LPXTG, where X signifies any amino acid but most commonly Ala, Ser, Glu (SEQ ID NO: 14), or a N-terminal pentaglycine repeat tag (SEQ ID NO: 18) is added to the envelope trimer gene. In a non-limiting embodiment, a C-terminal tag is LPXTGG, where X signifies any amino acid but most commonly Ala, Ser, Glu (SEQ ID NO: 15).

Table 1 shows a summary of sequences described herein.

| | | Amino acid, | |
|---|---|---|---|
| Name | Design | nucleic acid | FIG. |
| HV1301580_D230N_H289N_P291S; | Modified | Nt | 41 (SEQ ID NO: 95) |
| CH848.3.D1305.10.19_D949V3.DS.SOSIP_{—} | CH848.3.D1305.10.19 | aa | 42 (SEQ ID NO: |
| D230N_H289N_P291S (glycan hole | D949 | | 103) |
| filled) | | | |
| >HV1301502_D130SV1; JRFL_SOSIPv6_{—} | Modified | Nt | 41 (SEQ ID NO: 96) |
| V1_PNGS_D1305V1 (V1 loop from | CH848.3.D1305.10.19 | aa | 42 (SEQ ID NO: |
| 10.19) | D949 | | 104) |
| >HV1301405_D1305V1; CON- | Modified | Nt | 41 (SEQ ID NO: 97) |
| Schim.6R.DS.SOSIP.664_OPT_D1305V1 | CH848.3.D1305.10.19 | aa | 42 (SEQ ID NO: |
| (V1 loop from 10.19 isolate) | D949 | | 105) |
| >HV1301580_D230N_H289N_P291S; | Modified | Nt | 41 (SEQ ID NO: 98) |
| CH848.3.D1305.10.19_D949V3.DS.SOSIP_{—} | CH848.3.D1305.10.19 | aa | 42 (SEQ ID NO: |
| D230N_H289N_P291S (glycan holes | D949 | | 106) |
| filled) | | | |
| >HV1301580; CH848.3.D1305.10.19_{—} | 19CV3; DS SOSIP | Nt | 41 (SEQ ID NO: 99) |
| D949V3.DS.SOSIP (19CV3) | soluble trimer | aa | 42 (SEQ ID NO: |
| | | | 107) |
| >HV1301509; CH0848.3.d1305.10.19gp160 | Full length gp160 | Nt | 41 (SEQ ID |
| | | aa | NO: 100) |
| | | | 42 (SEQ ID NO: |
| | | | 108) |
| >HV1301503; | Chimeric (different | Nt | 41 (SEQ ID NO: |
| CH848.3.D1305.10.19ch.DS.SOSIP.664 | gp41) DS SOSIP | aa | 101) |
| | soluble trimer | | 42 (SEQ ID NO: |
| | | | 109) |
| >HV1301504; | v6 modifications | Nt | 41 (SEQ ID |
| CH848.3.D1305.10.19ch.SOSIPv6 | | aa | NO: 102) |
| | | | 42 (SEQ ID NO: |
| | | | 110) |
| >HV1301580_C_SORTA; | Sortase acceptor site | Aa | 43 (SEQ ID NO: |
| CH848.3.D1305.10.19_D949V3.DS.SOSIP_{—} | for nanoparticle | nt | 113) |
| C_SORTA | formation | | 43 (SEQ ID NO: |
| | | | 114) |

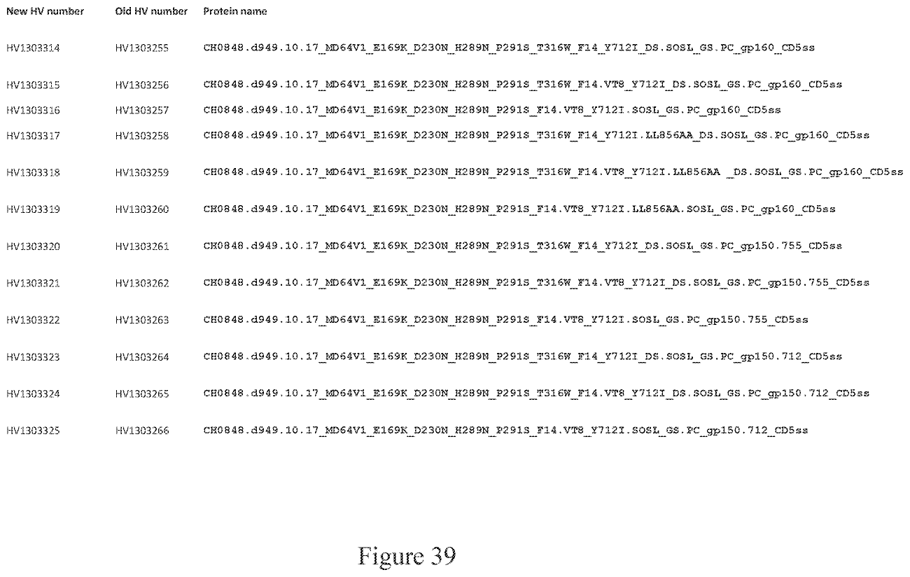

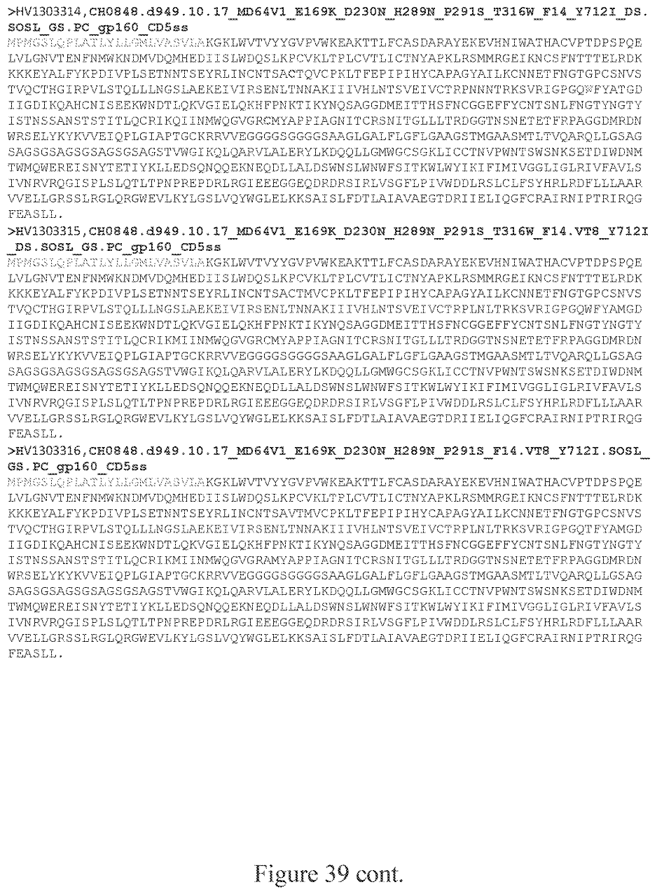

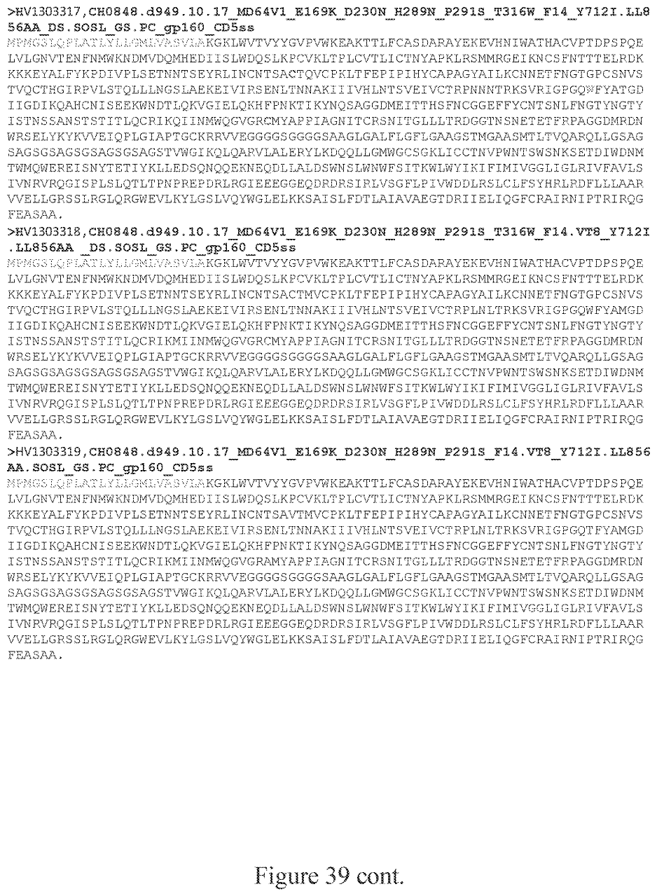

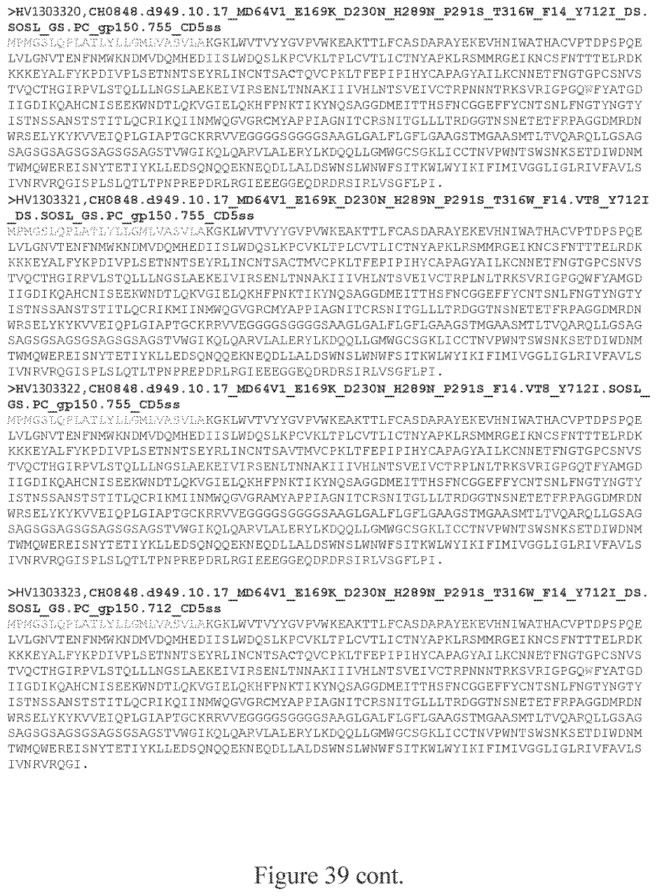

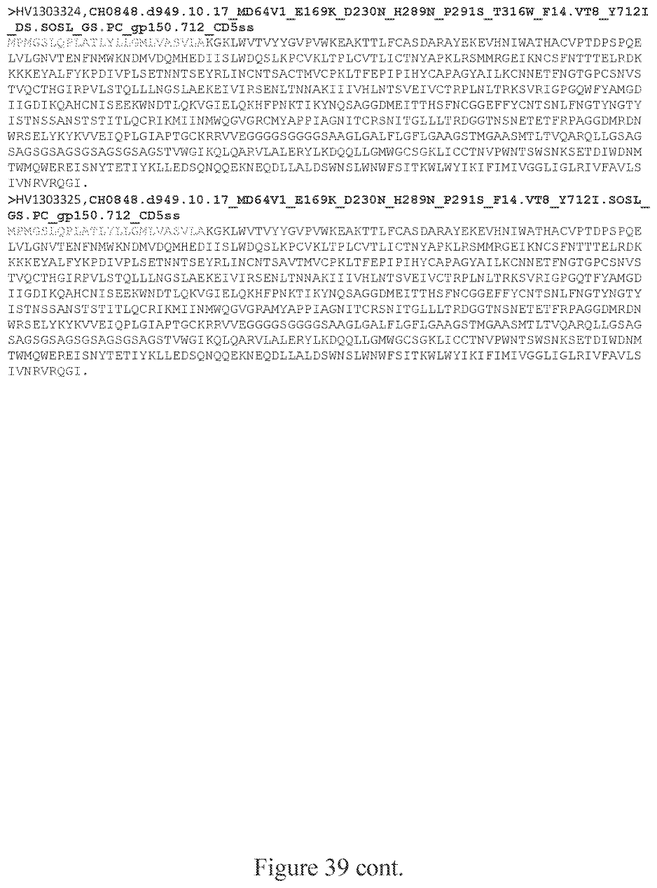

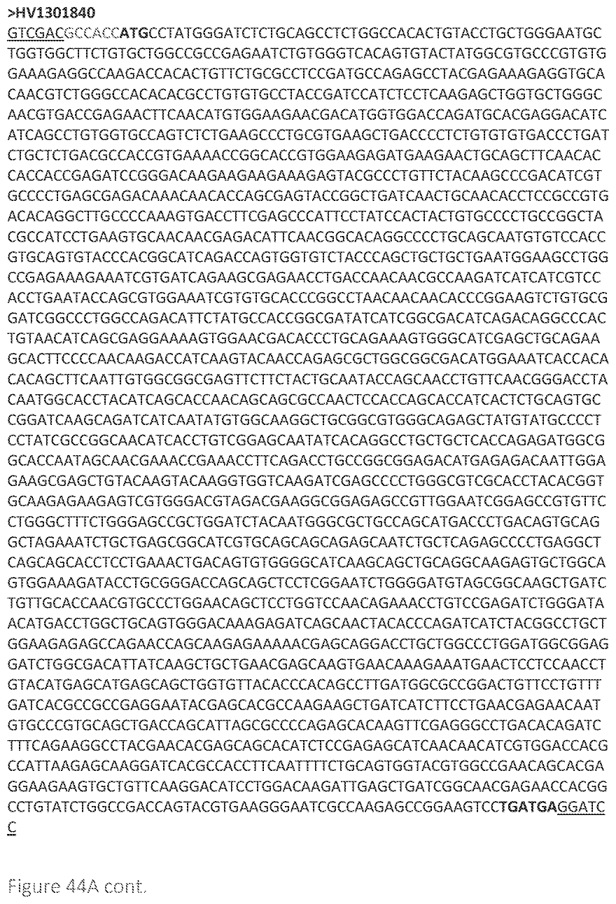

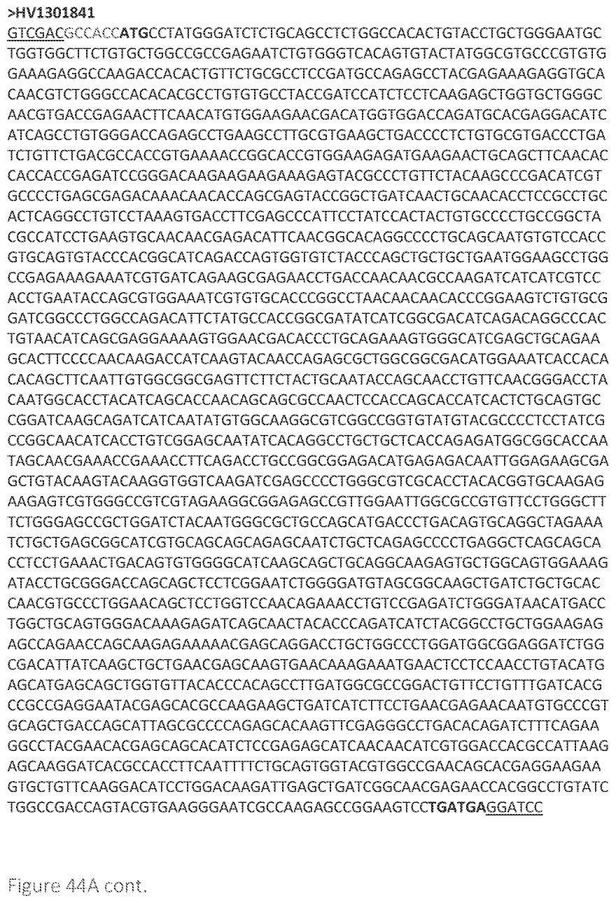

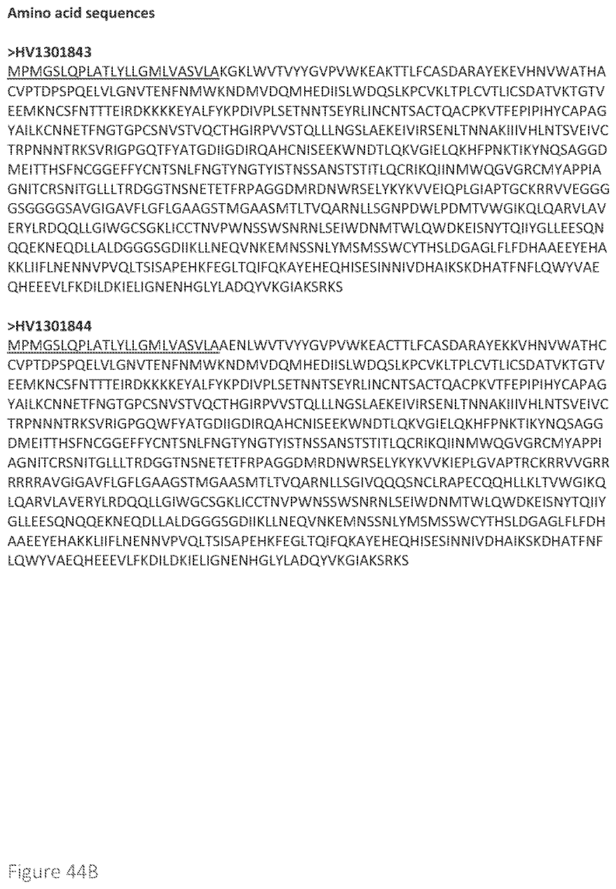

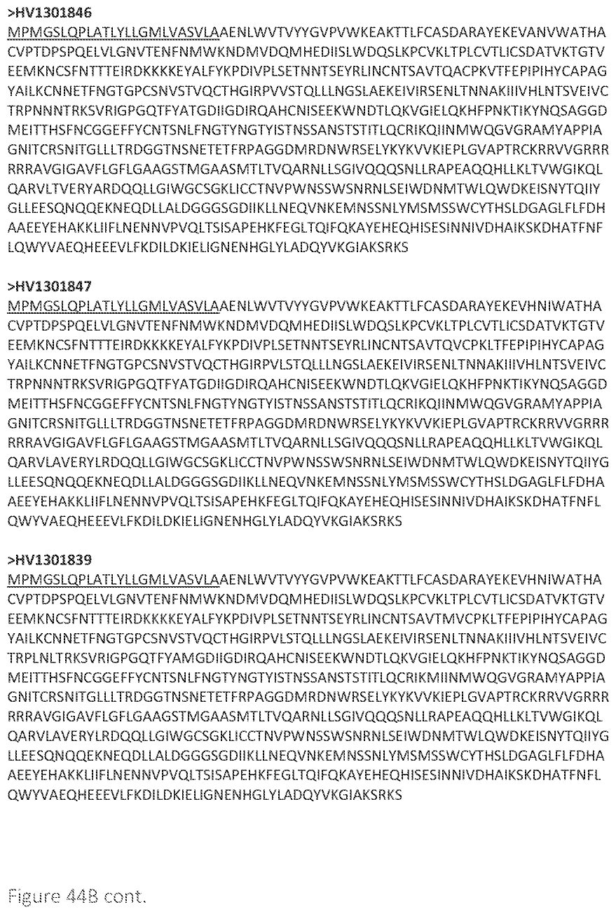

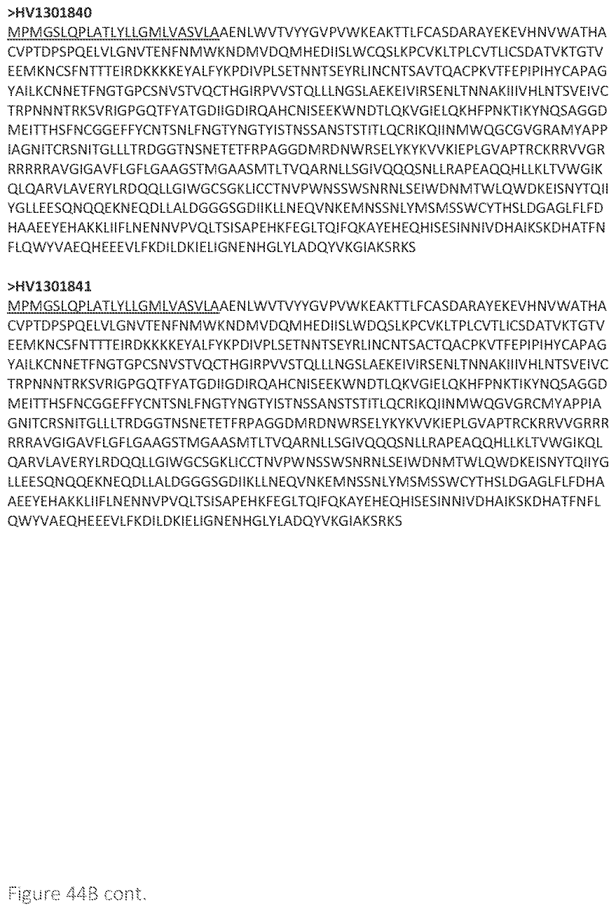

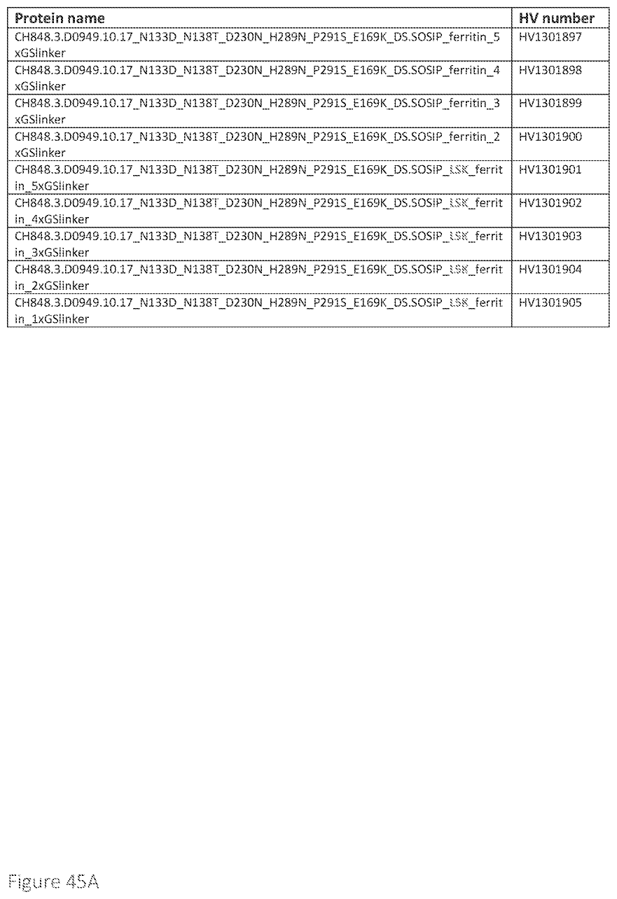

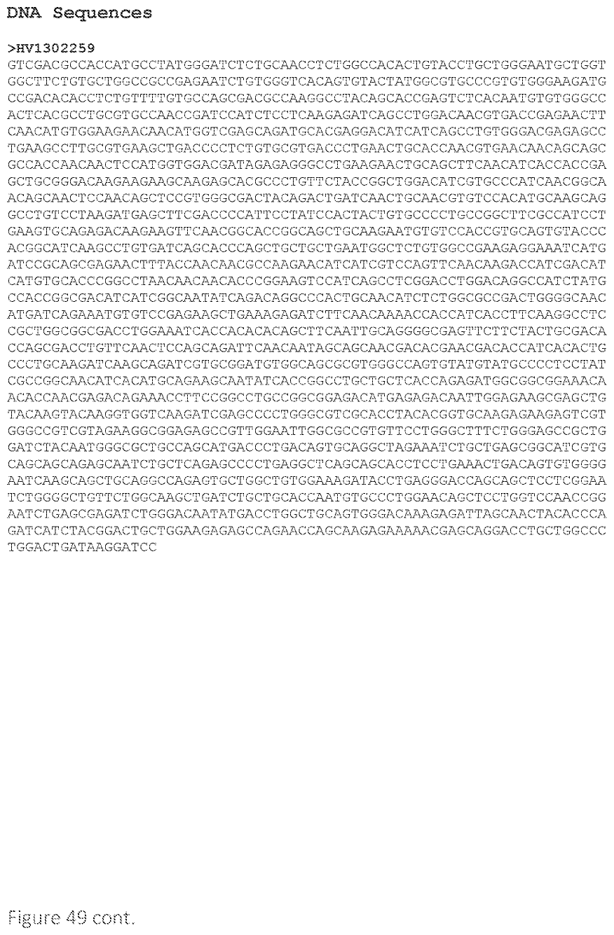

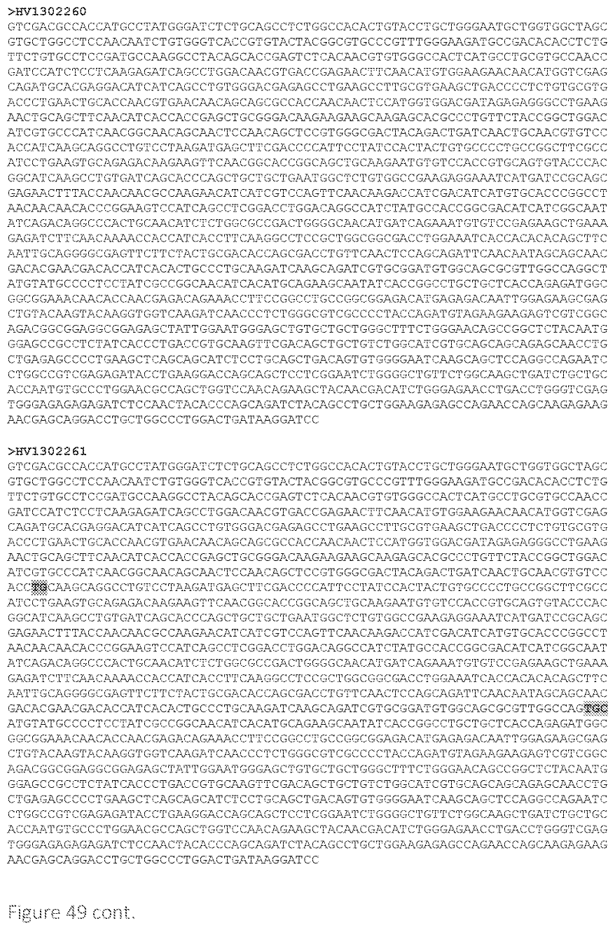

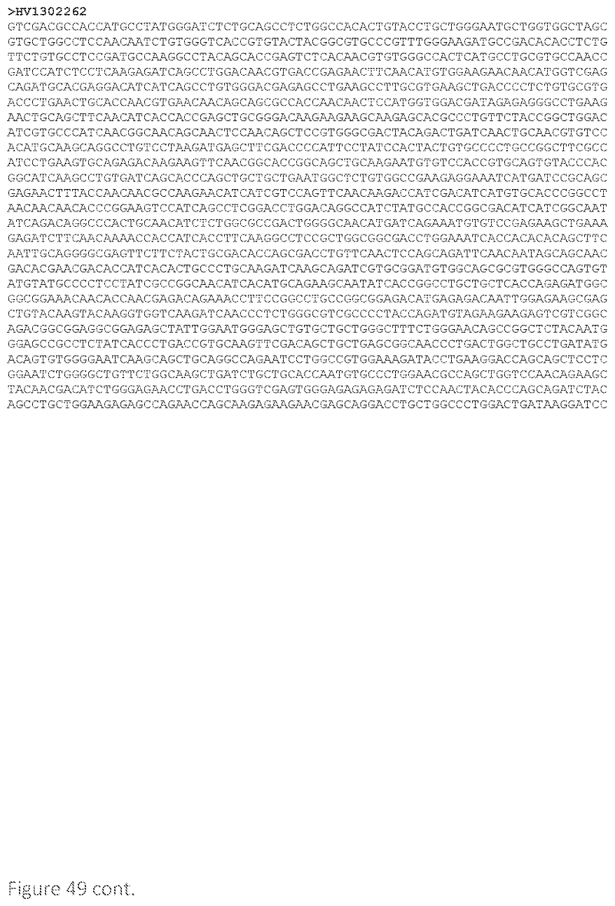

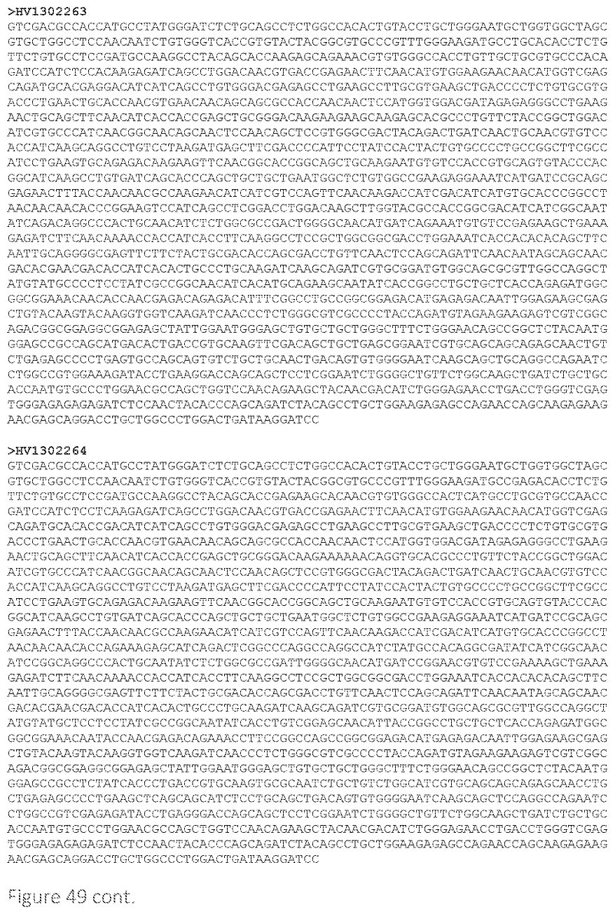

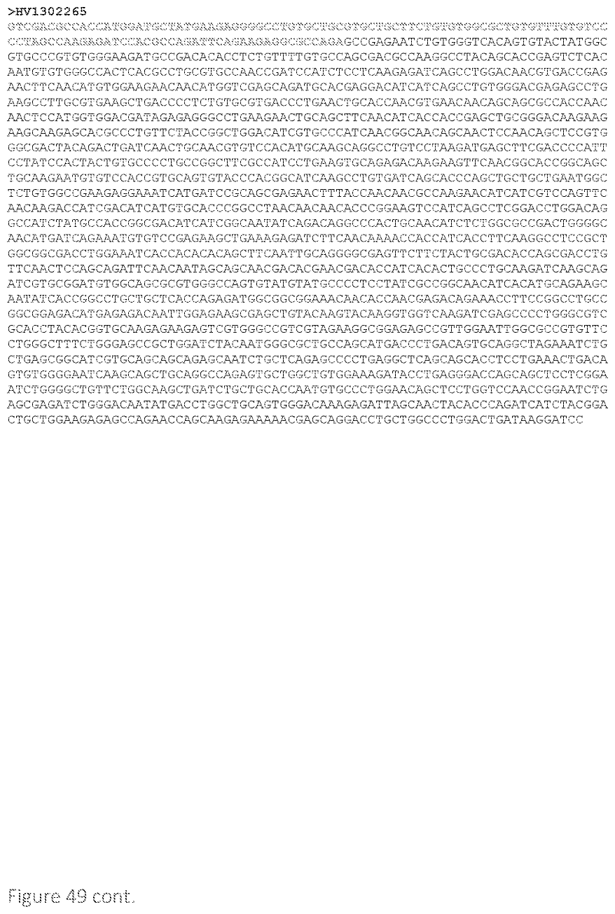

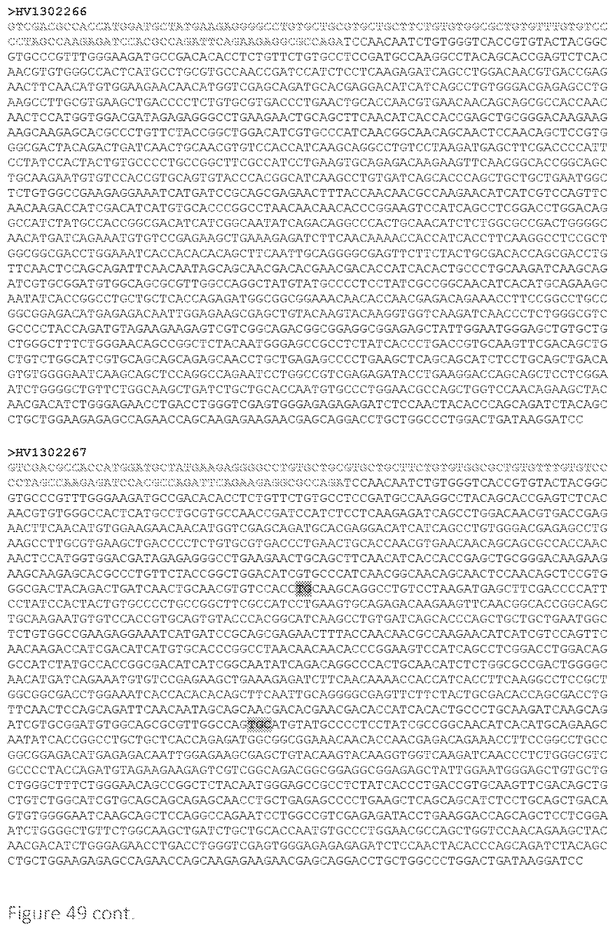

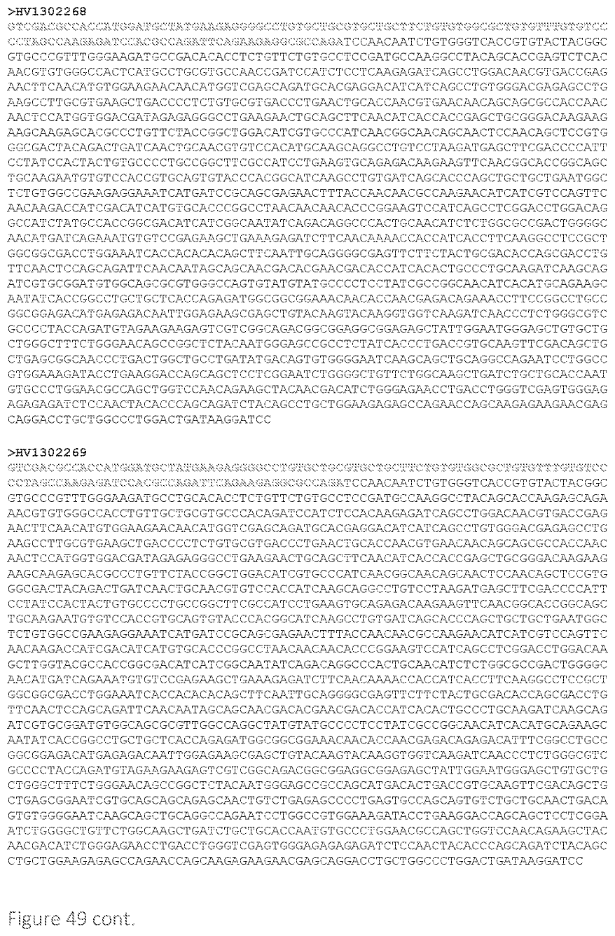

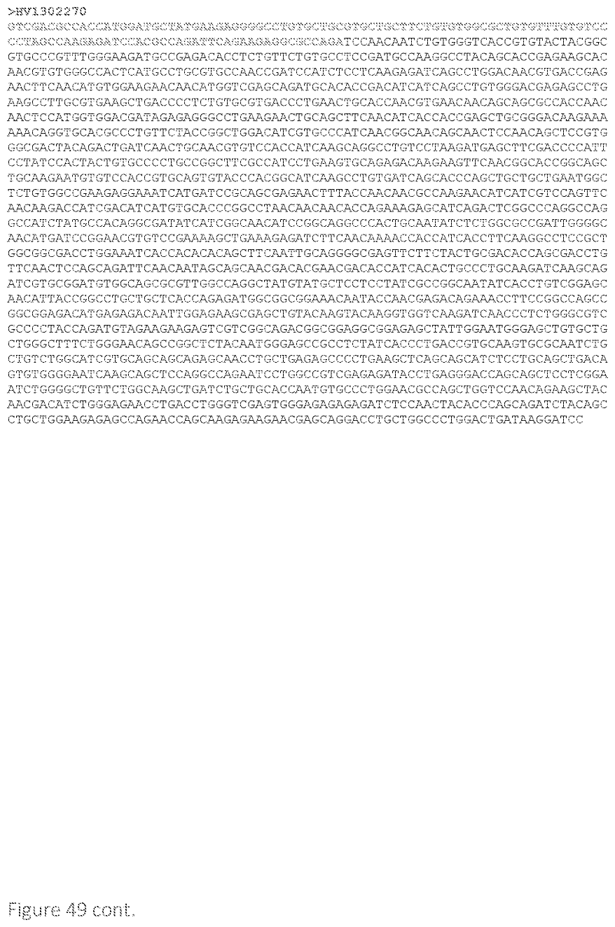

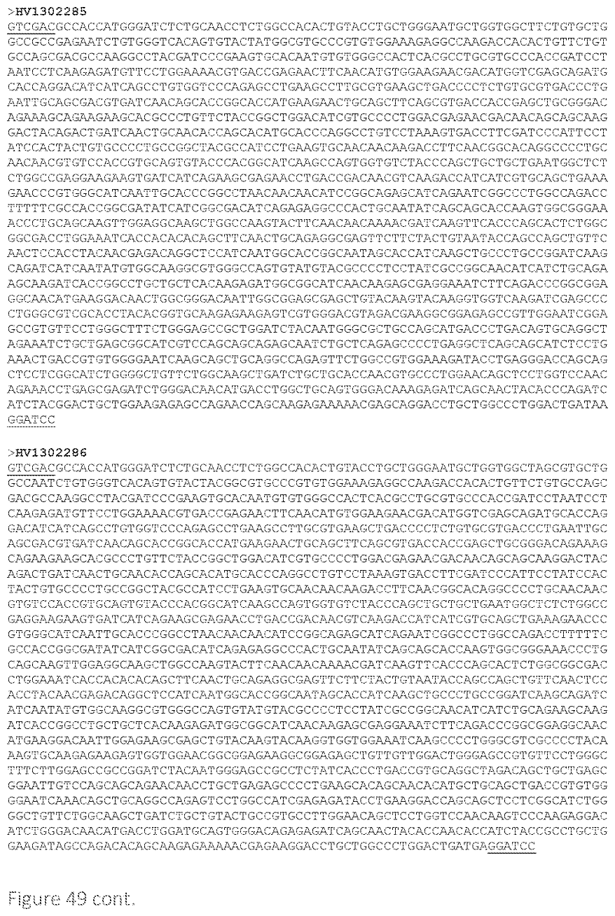

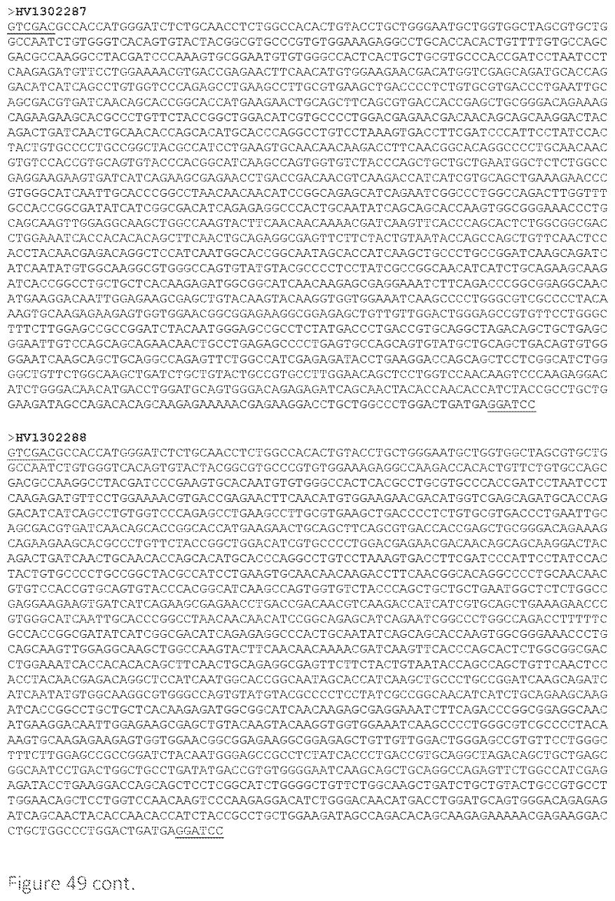

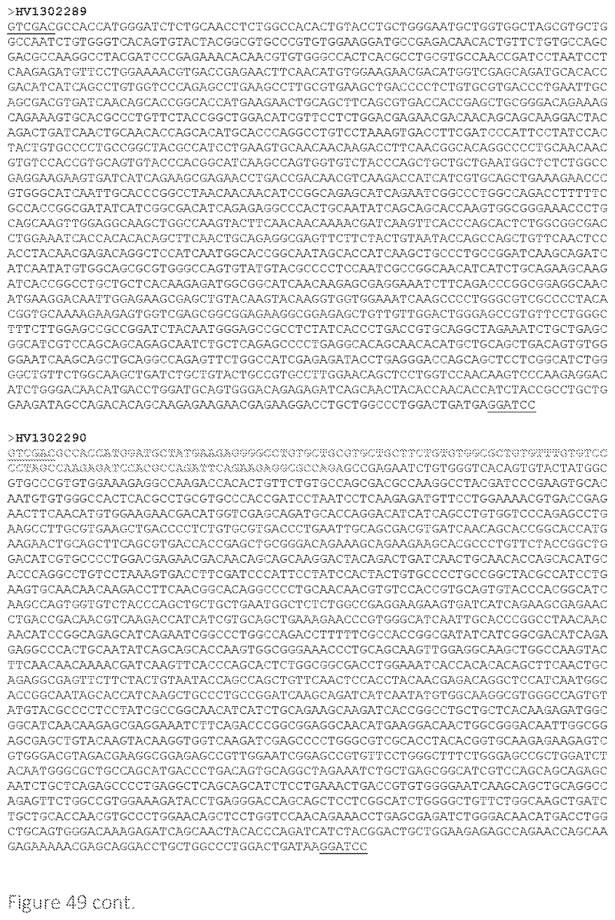

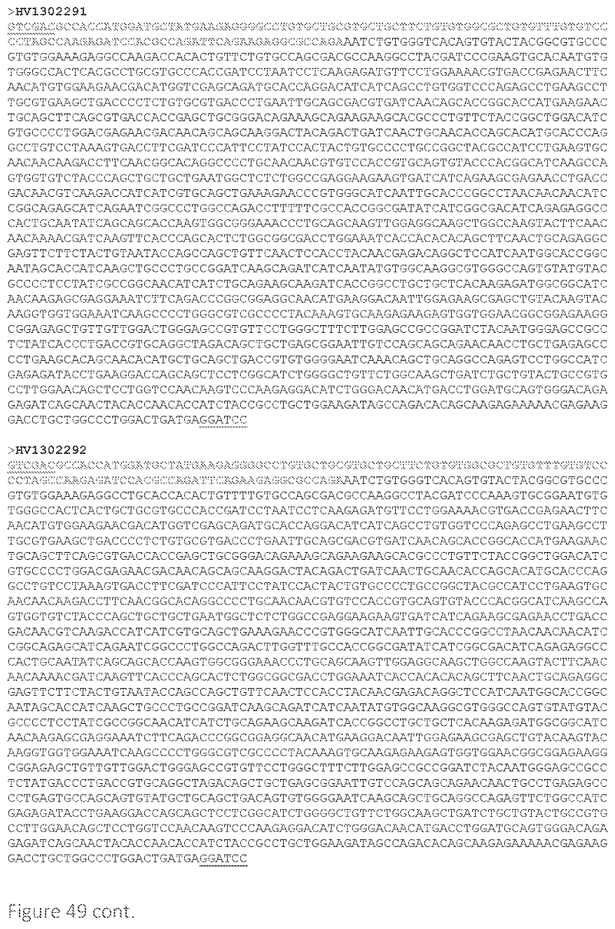

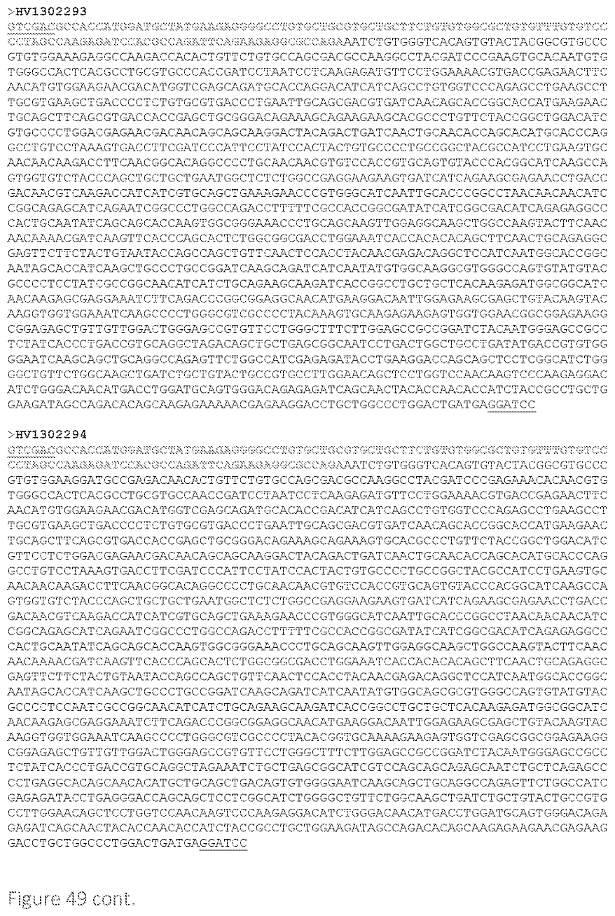

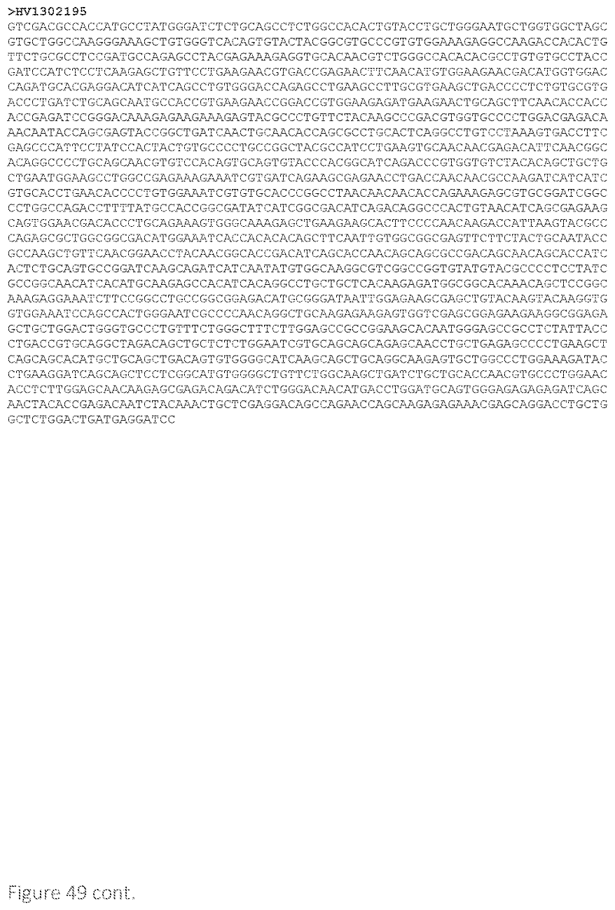

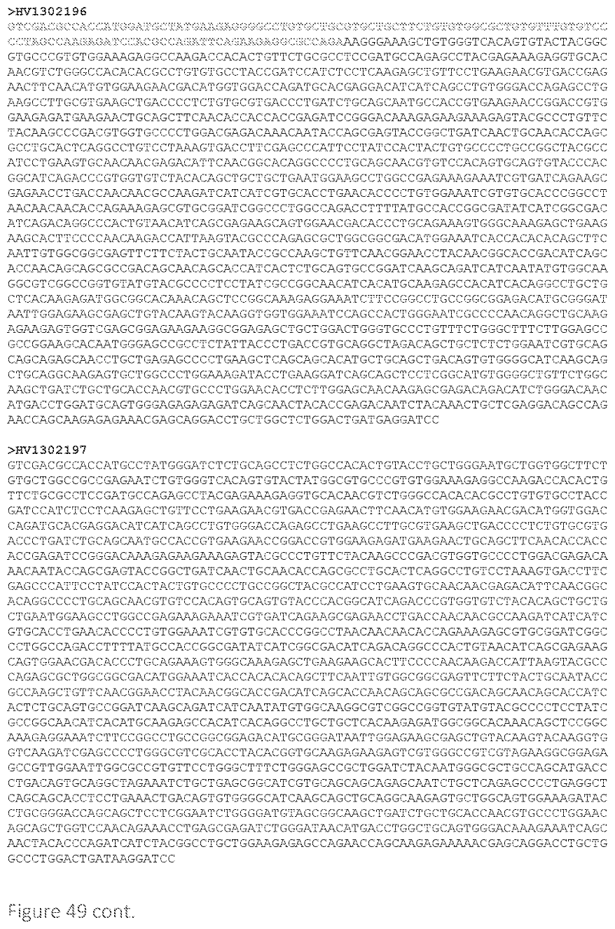

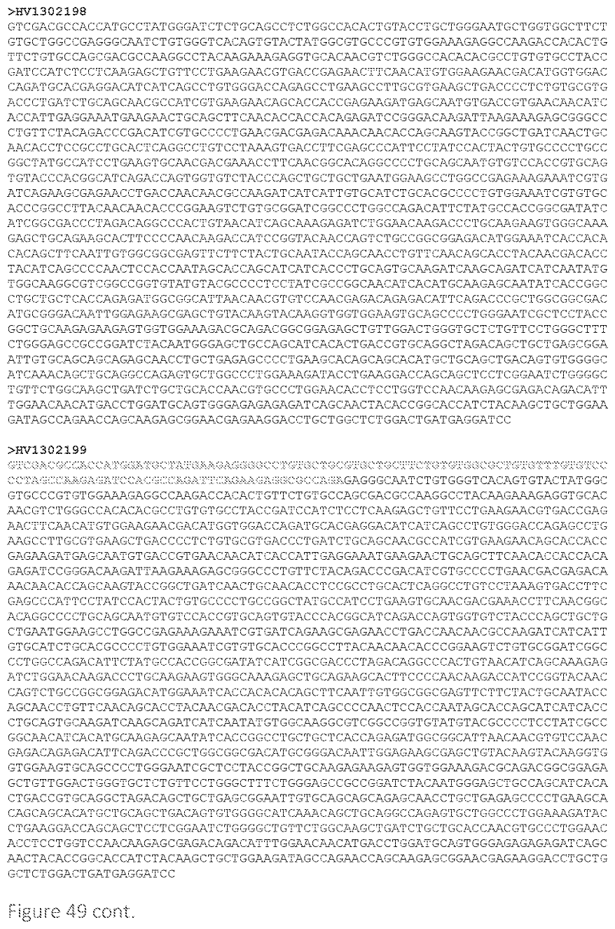

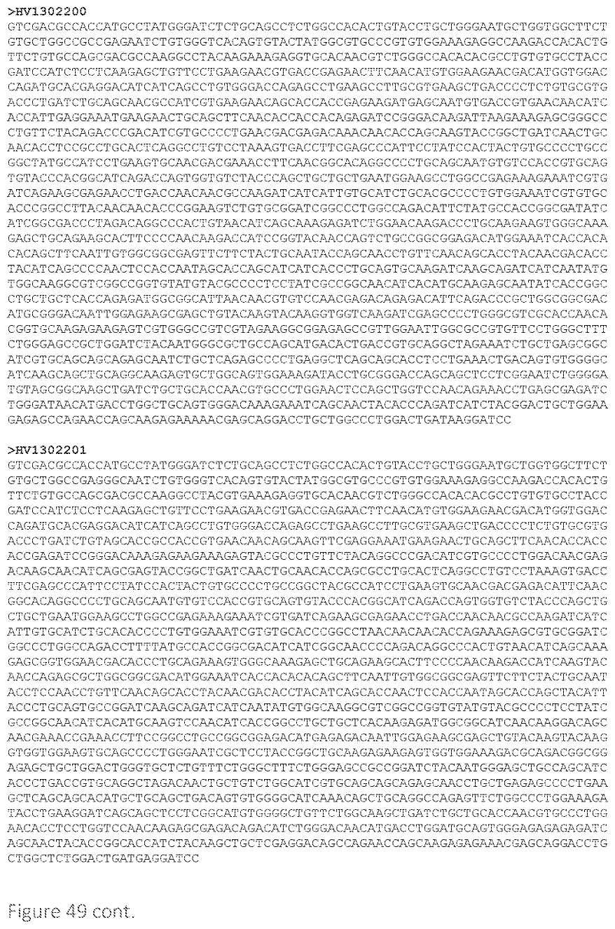

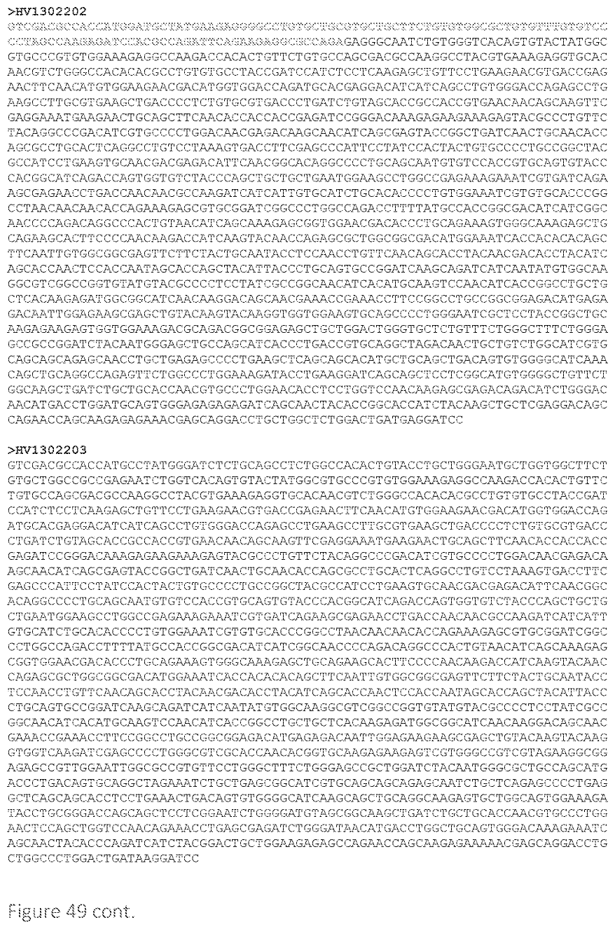

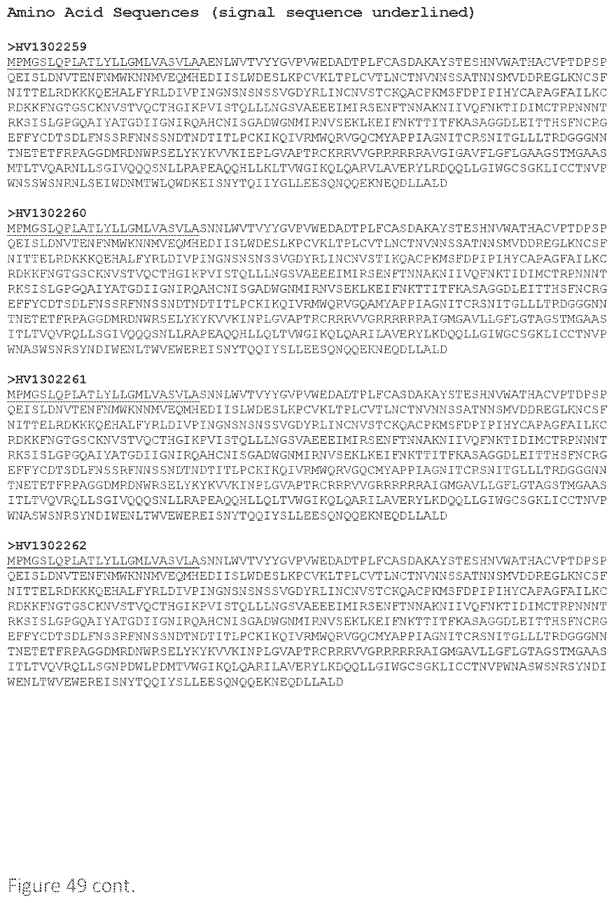

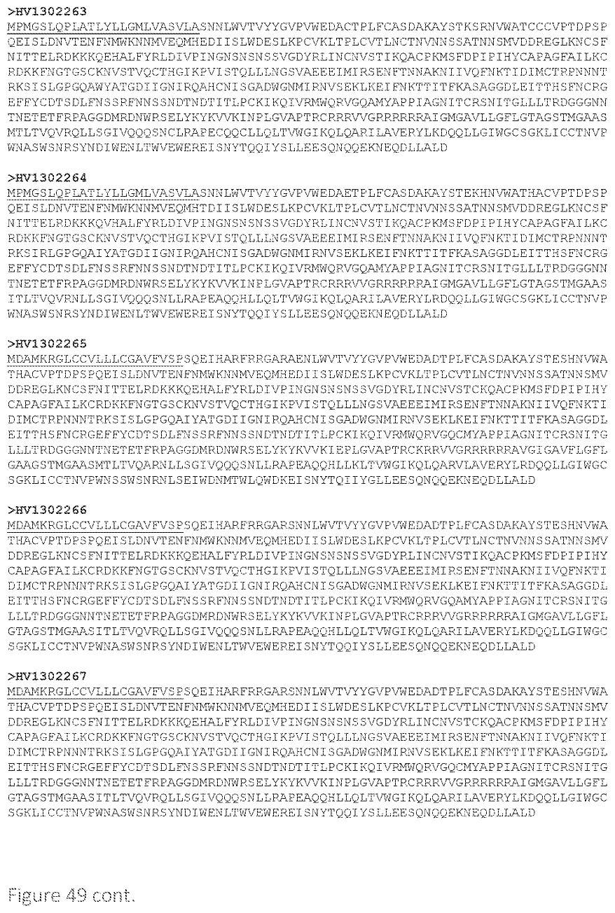

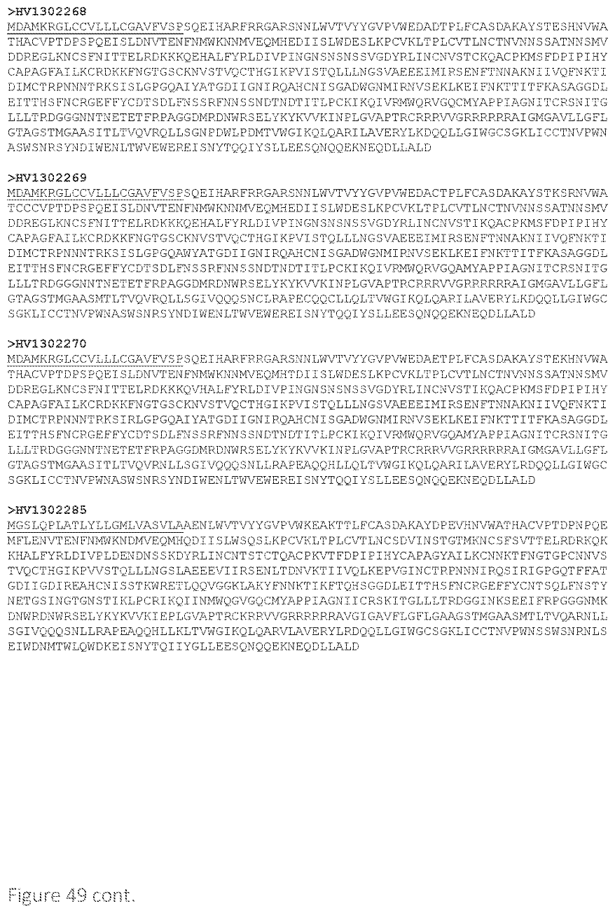

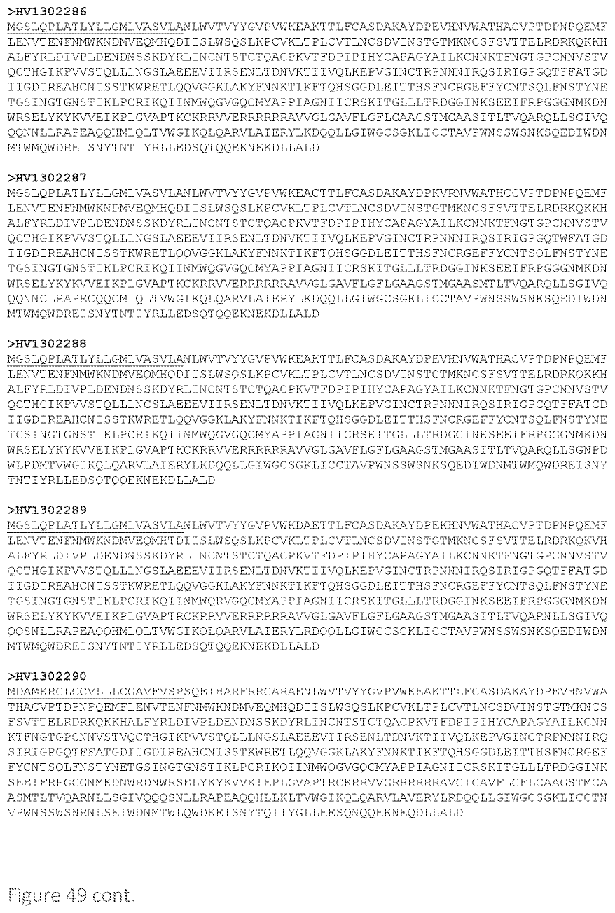

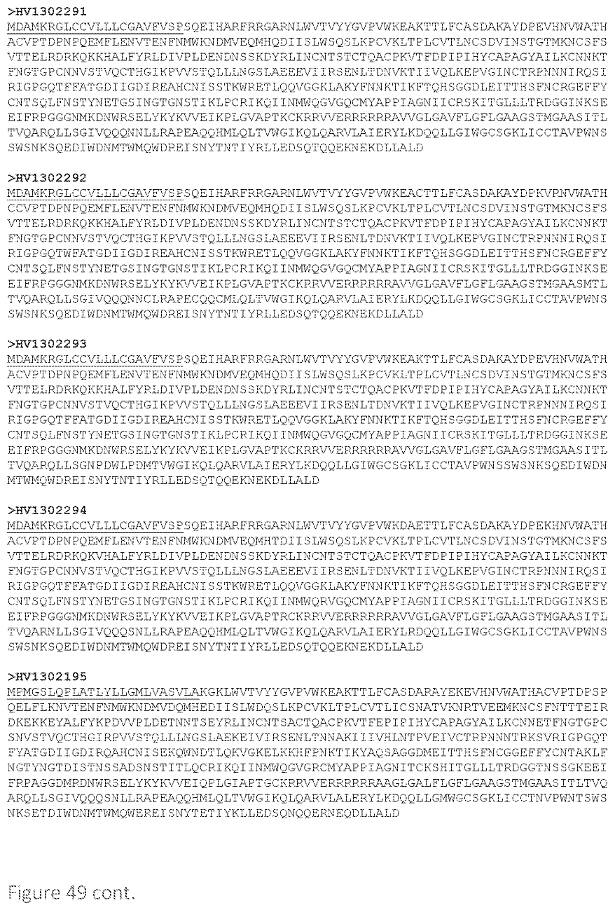

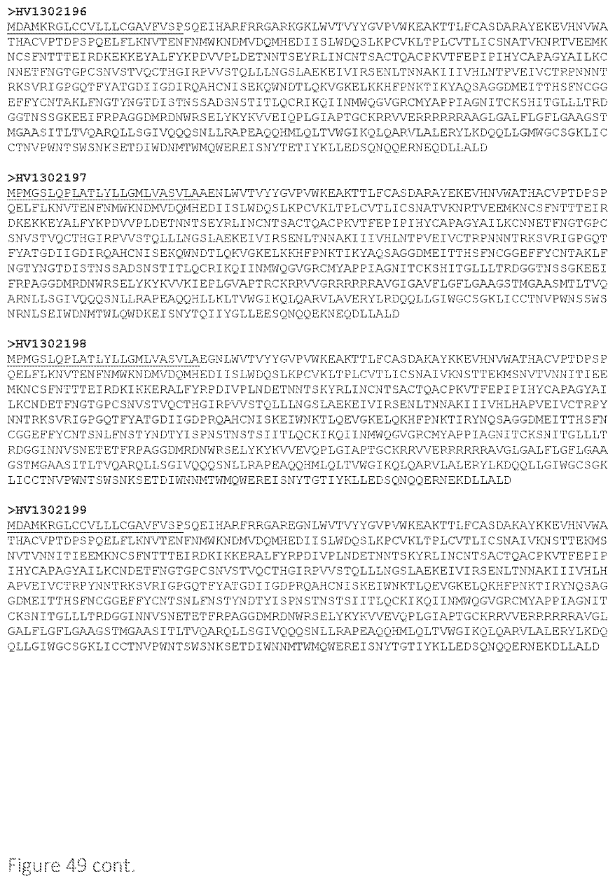

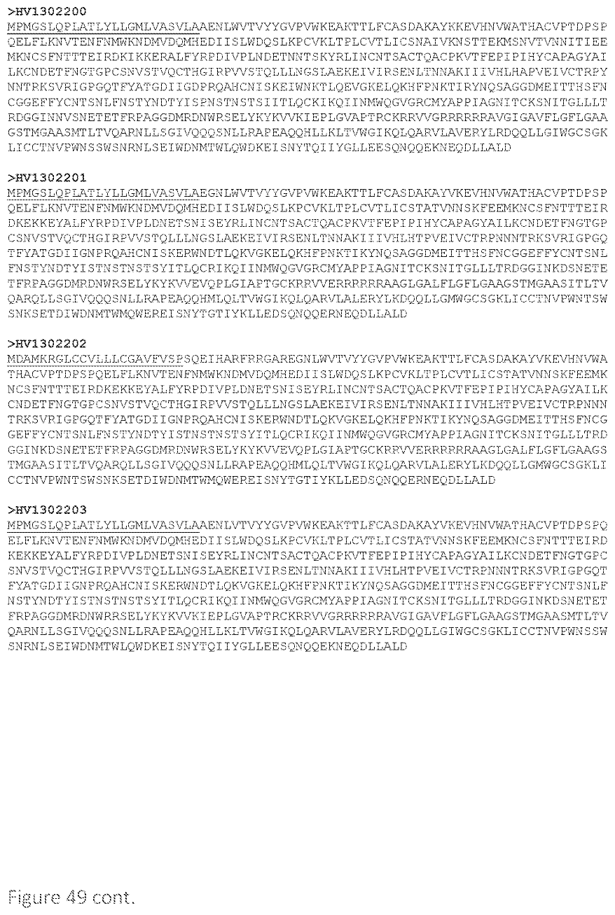

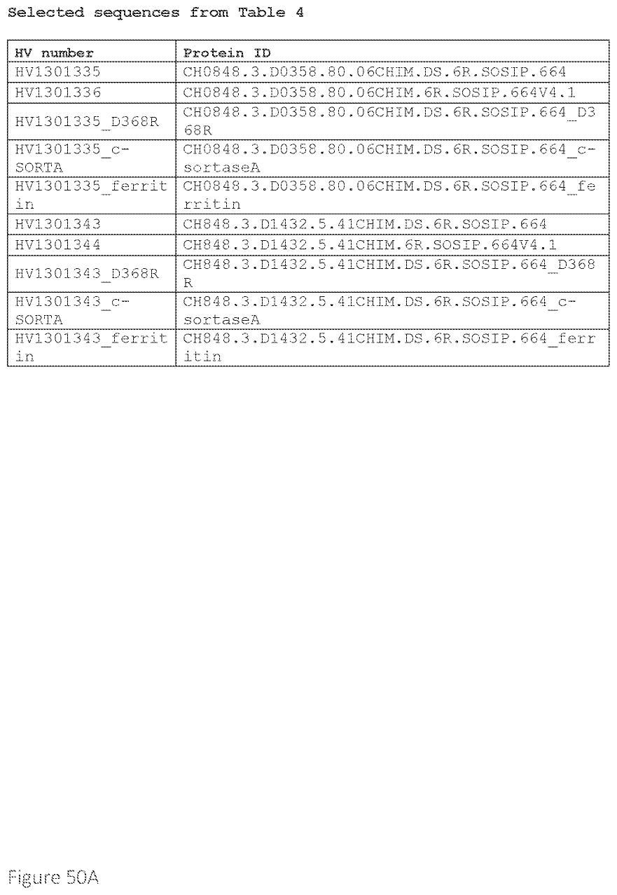

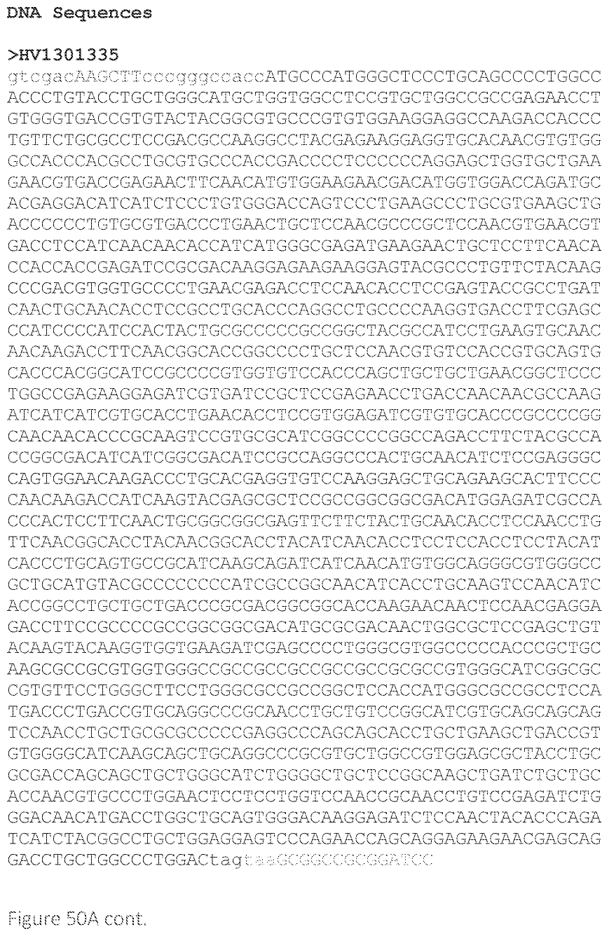

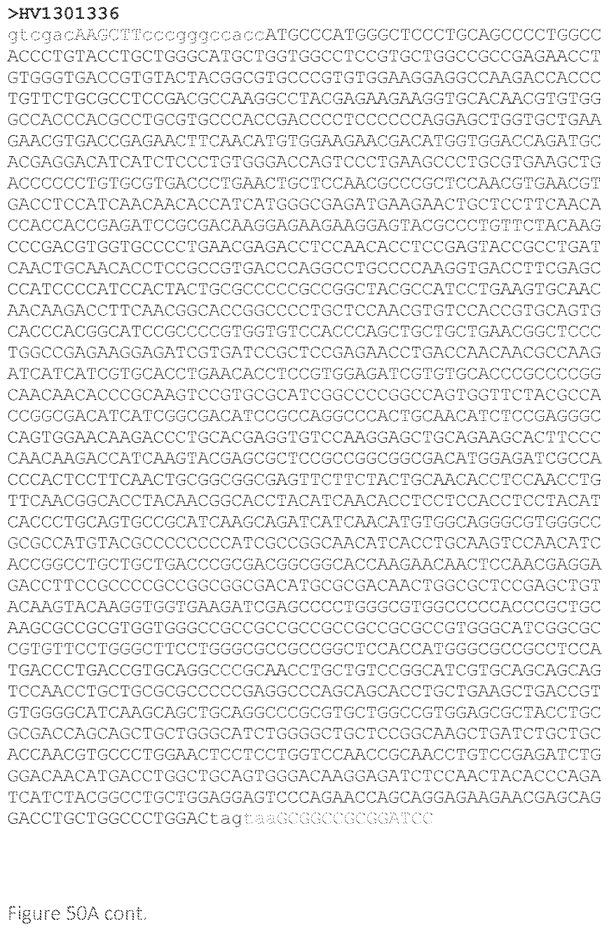

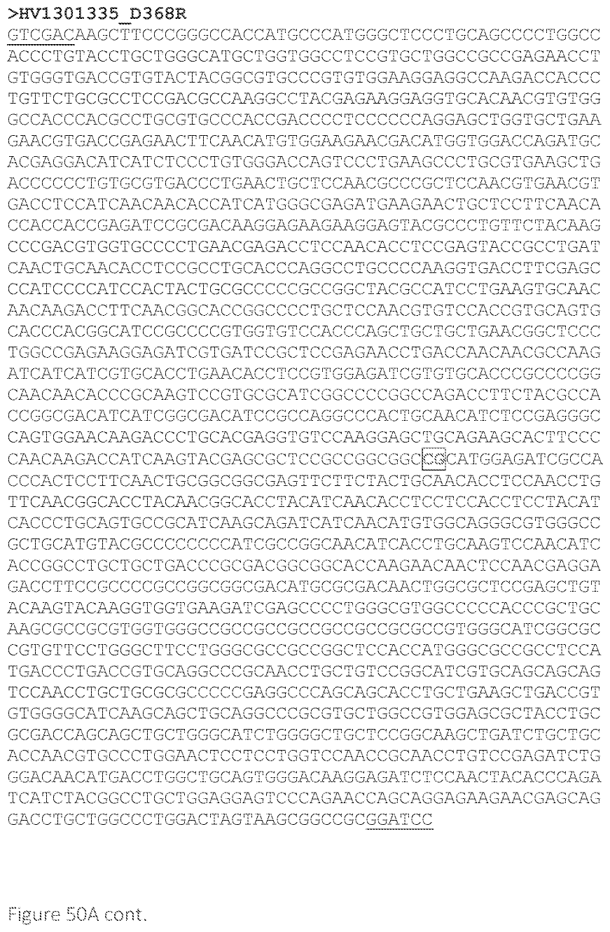

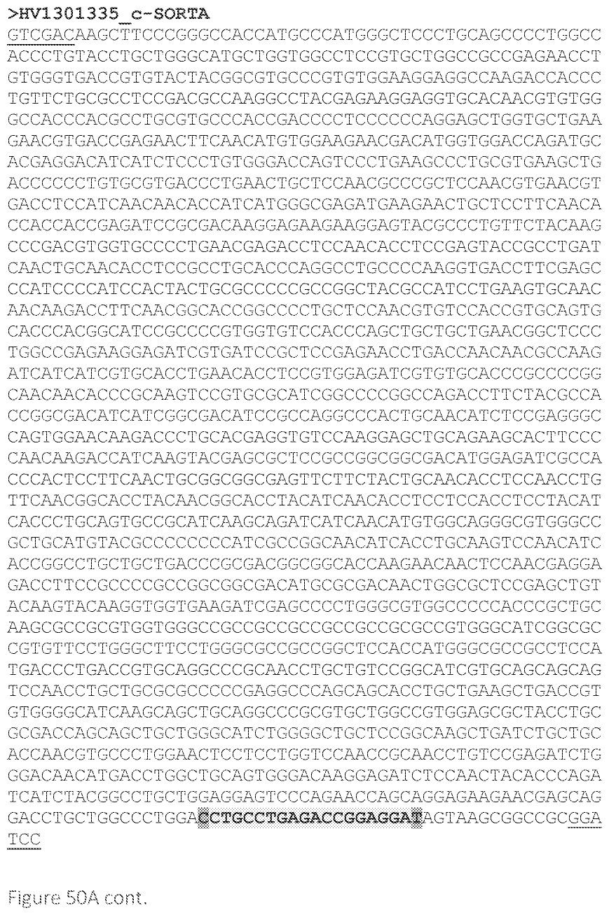

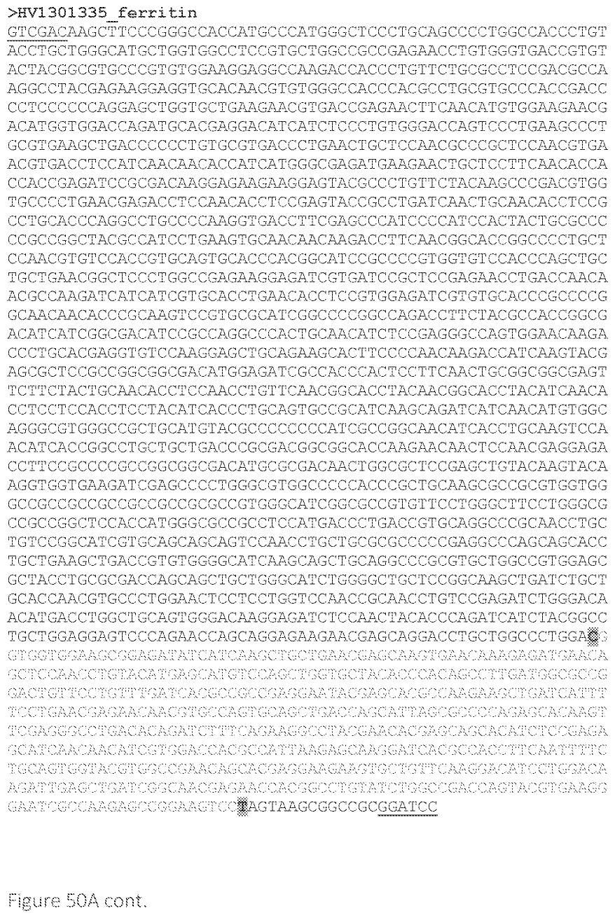

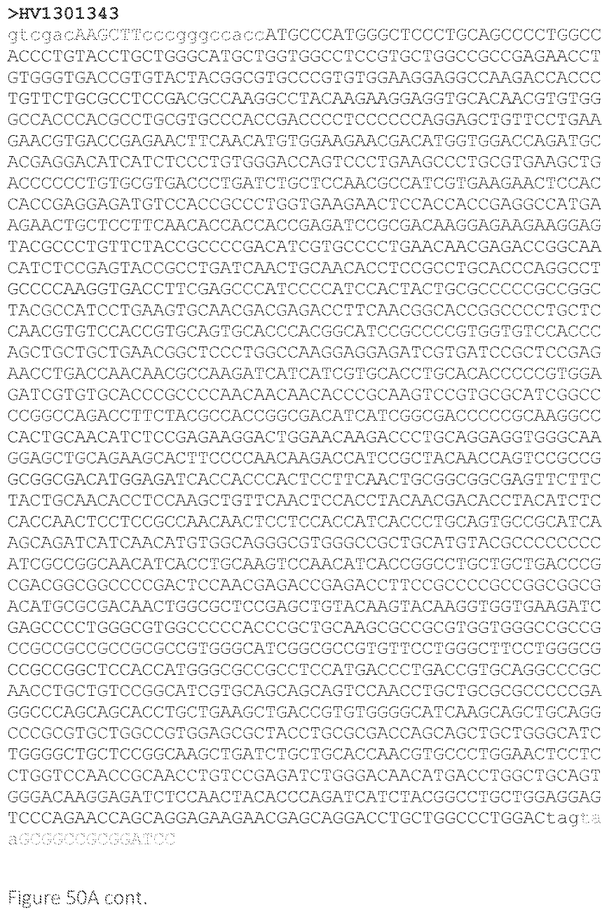

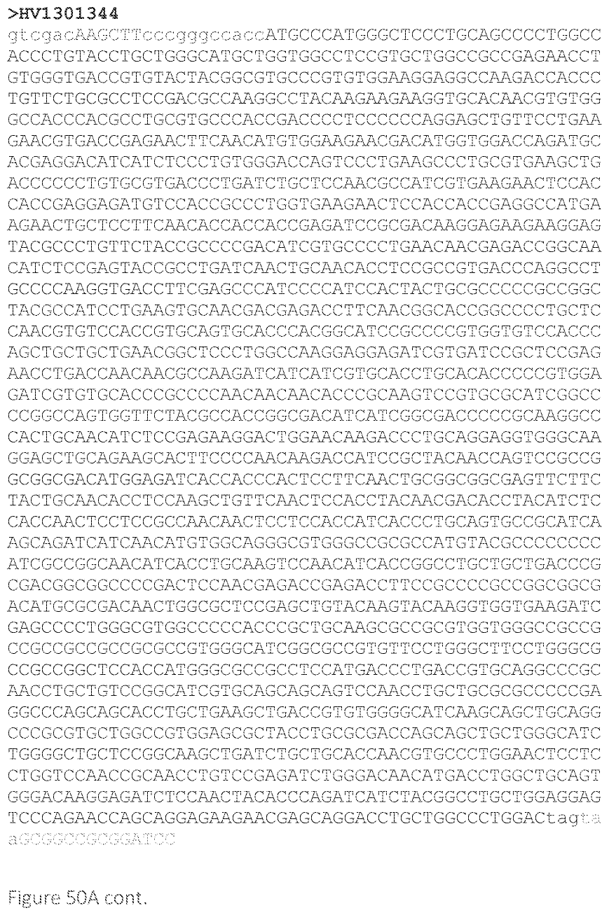

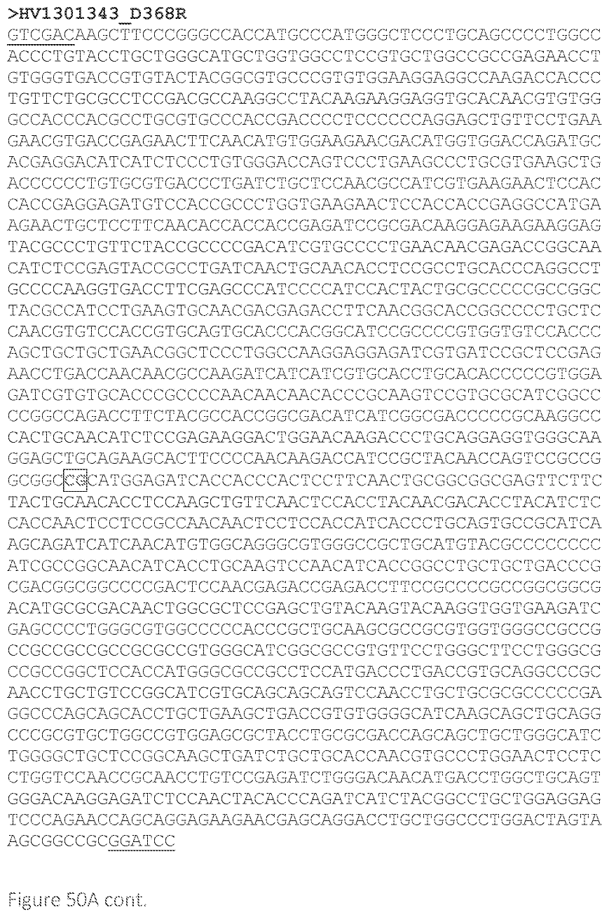

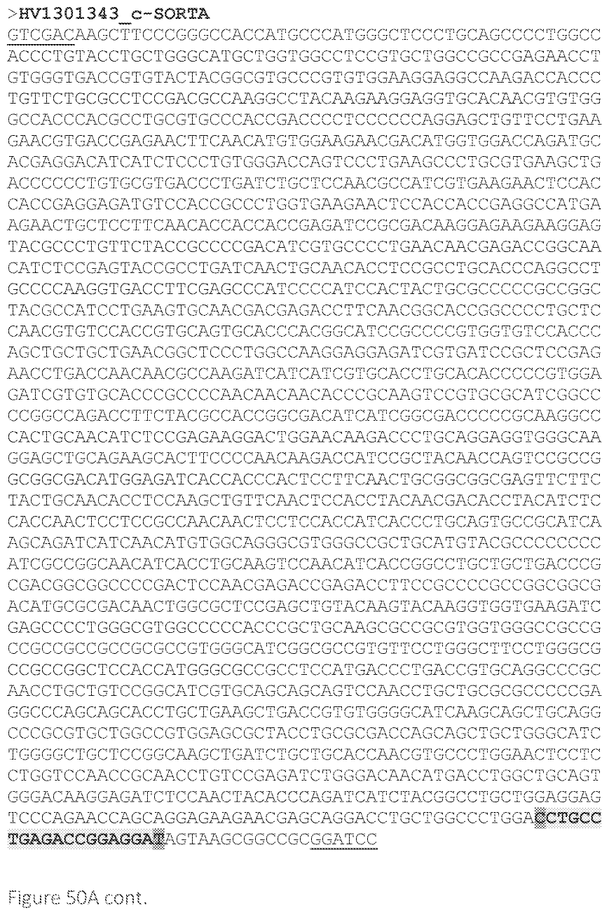

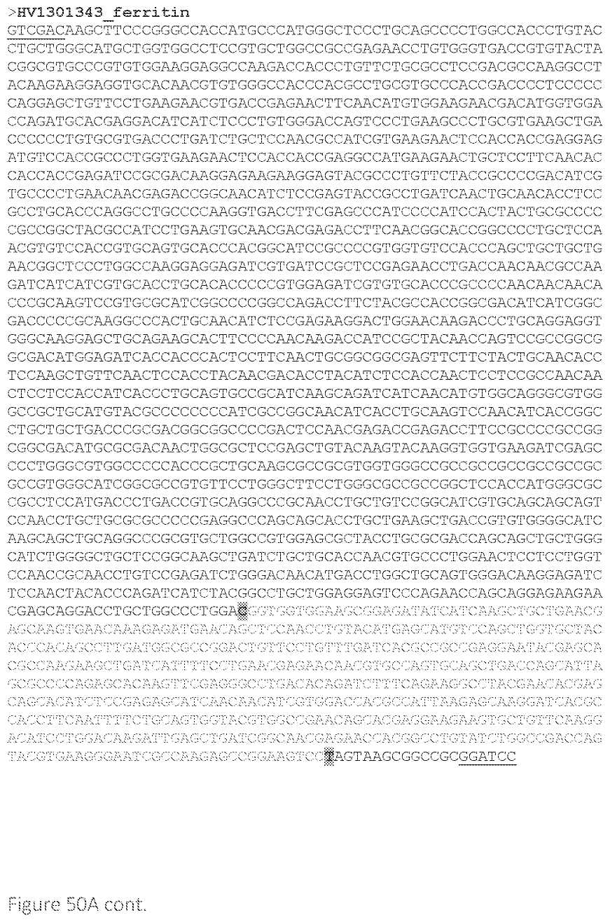

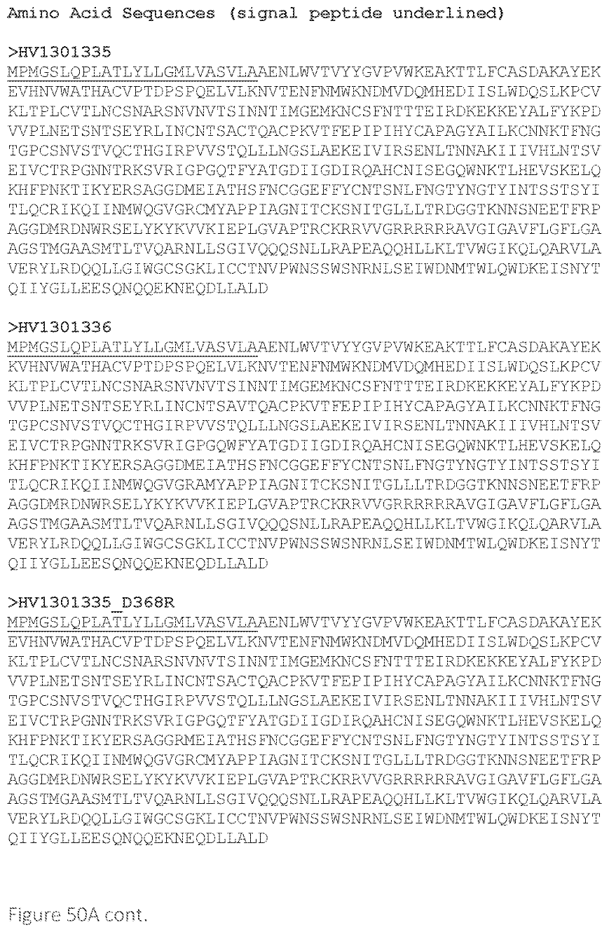

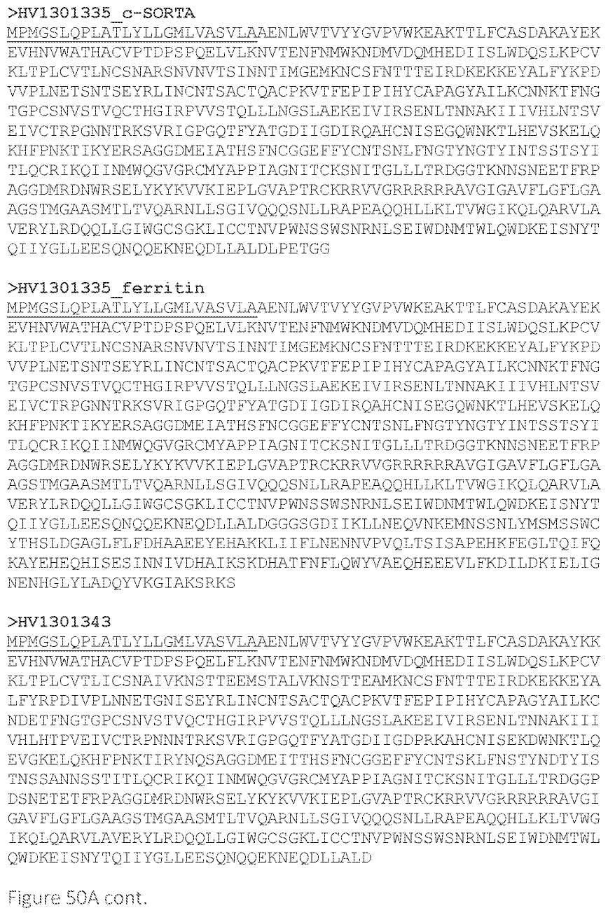

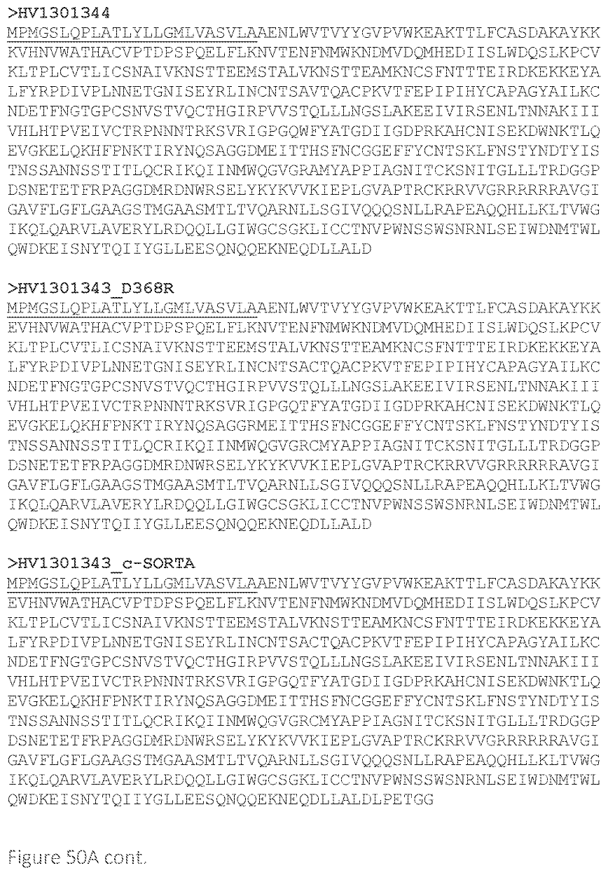

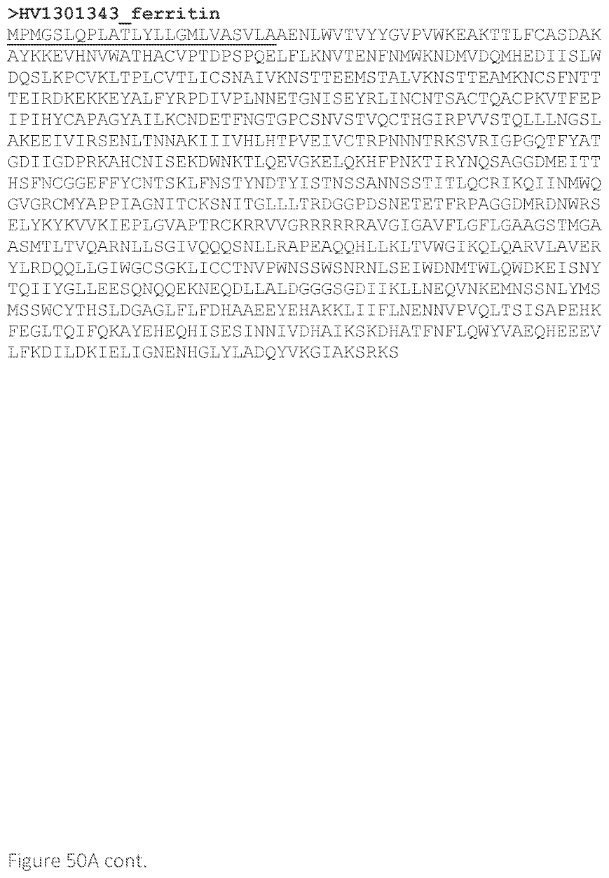

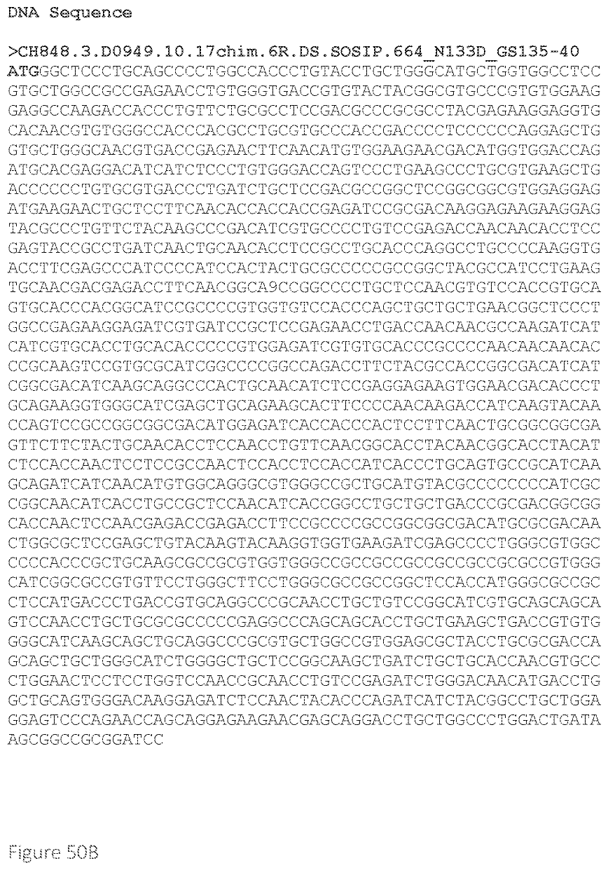

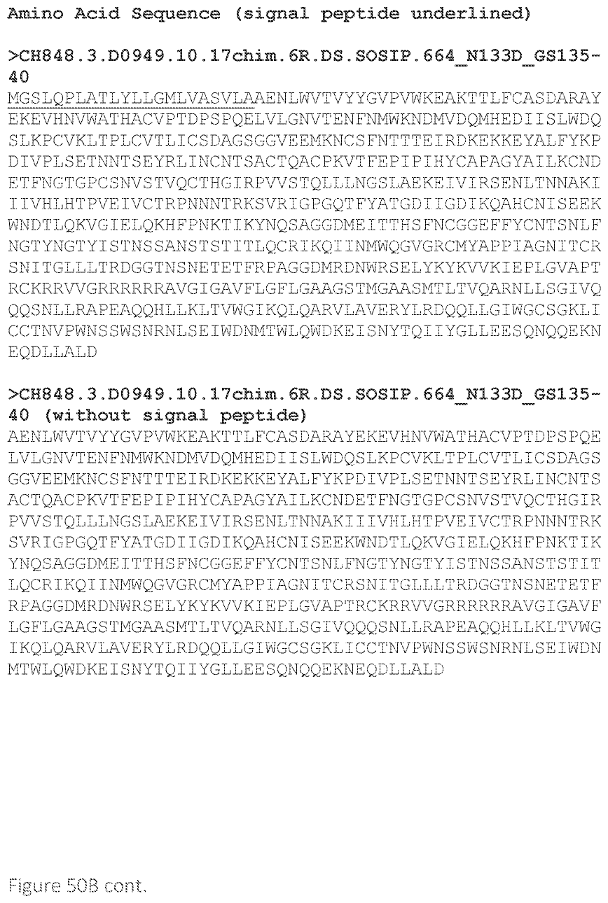

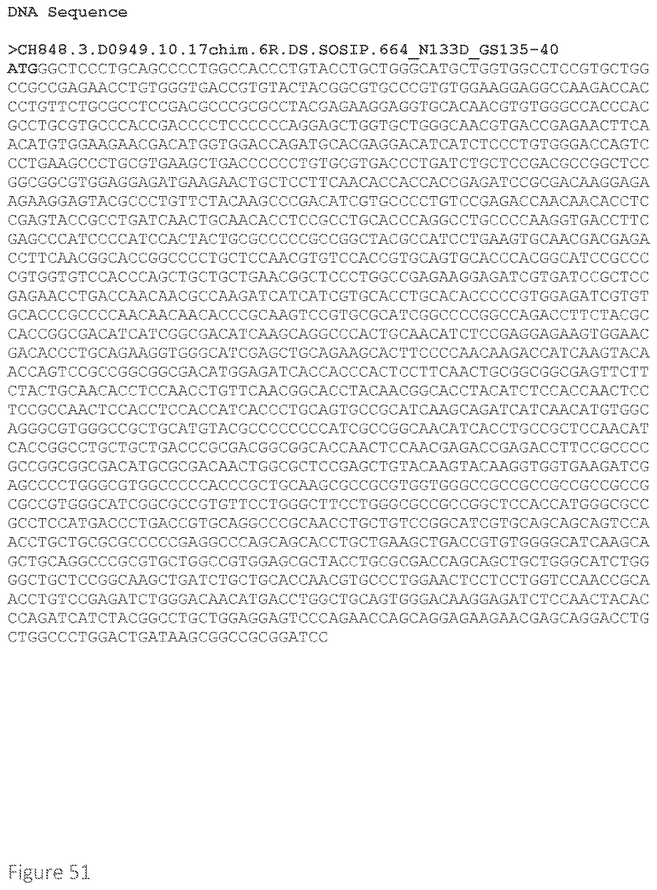

Table 2 shows a summary of modifications to envelopes described herein

| | | | V3 | UCA and |
|---|---|---|---|---|
| | | | glycosylation | other Ab |
| Envelope | FIG./SEQ ID No | V1 region | sites | binding |
| 10.17 | See | 17aa | N301 and N332 | |
| | US2020/0113997 | | | |
| | incorporated | | | |
| | herein by | | | |
| | reference in its | | | |
| | entirety including | | | |
| | FIGS. 39A-B and | | | |
| | SEQ ID NOs | | | |
| | disclosed therein | | | |
| 10.17 DT | See | 17aa N133D | N301 and N332 | DH270UCA |
| | US2020/0113997 | N138T effectively | | |
| | incorporated | lacks | | |
| | herein by | glycosylation sites | | |
| | reference in its | | | |
| | entirety including | | | |
| | FIG. 49A, 50B, | | | |
| | 51, 52A, 53D, 54A- | | | |
| | C, 77D-G, 77I and | | | |
| | SEQ ID NOs | | | |
| | disclosed therein | | | |
| 10.19 | FIG. 41 | 17aa V1 region | No | CH01 UCA |
| | | lacks N133 and | glycosylation | |
| | | N138 | sites at N295, | |
| | | glycosylation sites | N301, N332 | |
| 10.19 plus V3 loop of | FIG. 41, FIG. 43 | 17aa V1 region | Add V3 regions | CH01 UCA |
| 10.17 (19CV3) | | lacks N133 and | from 10.17 has | DH270UCA |
| | | N138 | five aa | VRC26 |
| | | glycosylation sites | difference from | UCA |
| | | | 10.19 | |
| 10.19 env based with fewer | See e.g., FIG. | | At least changes | |
| than five aa changes | 44A-B | | #2, 4, 5, and/or | |
| compared to 19CV3; | | | “GDIR” | |
| “GDIR/K” | | | sequence (SEQ | |
| | | | ID NO: 1) | |
| Ferritin Linker | See e.g., FIG. 45A-B | | | |
| E169K | See e.g., FIG. 44A-B | | | |
| Glycan hole filled | See e.g., FIG. 44A-B. | | | |
| | 44A-B, 44A-B | | | |
| GS design | See e.g. FIG. 50A-B | | | |

DH270 light chain binds to N301 glycan. In some embodiments, a N301 gly site is used (e.g. change #2 in row 5 of Table 2, supra).

DH270 heavy chain binds to N332 glycan. In some embodiments, a N332 gly site is used (e.g. changes #4 and #5 in row 5 of Table 2, supra).

V3 glycan Abs bind GDIR (SEQ ID NO: 1). In some embodiments, a change #3 to “GDIR” (SEQ ID NO: 1) is needed (e.g. “GDIR” (SEQ ID NO: 1) sequence in row 5 of Table 2, supra).

GDIR/K motif: V3-glycan broadly neutralizing antibodies typically contact the c-terminal end of the third variable region on HIV-1 envelope. There are four amino acids, Gly324, Asp325, Ile326, and Arg327, bound by V3-glycan neutralizing antibodies. While Arg327 is highly conserved among HIV-1 isolates, Lys327 also occurs at this site. The CH848.3.D0949.10.17 isolate naturally encodes the less common Lys327. In contrast to CH848.3.D0949.10.17 with the Lys327, the precursor antibody of the DH270 V3-glycan broadly neutralizing antibody lineage barely binds to CH848.3.D0949.10.17 encoding Arg327. Thus, Arg327 is critical for the precursor to bind and the lineage of neutralizing antibodies to begin maturation. However, somatically mutating antibodies on the path to developing neutralization breadth bind better to Env encoding Arg327. Thus, Env must encode Lys327 to initiate DH270 lineage development. However, to best interact with affinity maturing DH270 lineage members the Env should encode Arg327. Thus, a plausible vaccine regimen to initiate and select for developing bnAbs would include a priming immunogen encoding, Lys327 and a boosting immunogen encoding Arg327. The Arg327 boosting immunogen would optimally target the affinity maturing DH270 lineage members, while not optimally binding the DH270 antibodies that lack affinity maturation. Non-limiting embodiments of vaccination regimens could include: priming with CH848.3.D0949.10.17 based envelope design also with Lys327, followed by administering of

CH848.3.D0949.10.17 based envelope design with Arg327. Non-limiting embodiments of vaccination regimens could include: priming with 19CV3 based envelope design also with Lys327, followed by administering of CH848.3.D0949.10.17 based envelope design with Arg327.

E169K modification: One approach to designing a protective HIV-1 vaccine is to elicit broadly neutralizing antibodies (bnAbs). However, bnAbs against two or more epitopes will likely need to be elicited to prevent HIV-1 escape. Thus, optimal HIV-1 immunogens should be antigenic for multiple bnAbs in order to elicit bnAbs to more than one epitope. The CH848.D949.10.17 HIV-1 isolate was antigenic for V3-glycan antibodies but lacked binding to VIV2-glycan antibodies. Not all viruses from the CH848 individual lacked binding to V1V2-glycan antibodies. For example, the CH848.D1305.10.19 isolate bound well to VIV2-glycan antibody PGT145. We compared the sequence of CH848.D949.10.17 and CH848.D1305.10.19 in the region that is contacted by V1V2-glycan antibodies in crystal structures (McLellan J S, Pancera M, Carrico C, Gorman J, Julien J P, Khayat R, et al. Structure of HIV-1 gp120 V1/V2 domain with broadly neutralizing antibody PG9. Nature. 2011; 480 (7377): 336-43). Interestingly, the CH848.D949.10.17 and CH848.D1305.10.19 differed in sequence at a known contact site for VIV2-glycan antibodies-position 169 (Doria-Rose N A, Georgiev I, O'Dell S, Chuang G Y, Staupe R P, Mclellan J S, et al. A short segment of the HIV-1 gp 120 V1/V2 region is a major determinant of resistance to V1/V2 neutralizing antibodies. J Virol. 2012; 86 (15): 8319-23). It has been previously shown that mutation of lysine at position 169 eliminates binding to V1V2-glycan antibody PG9 (Doria-Rose N A, Georgiev I, O'Dell S, Chuang G Y, Staupe R P, McLellan J S, et al. A short segment of the HIV-1 gp120 V1/V2 region is a major determinant of resistance to V1/V2 neutralizing antibodies. J Virol. 2012; 86 (15): 8319-23). CH848.D1305.10.19 sequence encoded a lysine at position 169 whereas CH848.D949.10.17 sequence encoded a glutamate. Thus, we changed the glutamate (E) to lysine (K) at position 169 of CH848.D949.10.17. This single change in CH848.D949.10.17 enabled VIV2-glycan antibody binding to the envelope. Thus, the E169K adds the V1V2-glycan epitope to the other bnAb epitopes present on CH848.D949.10.17-based envelopes. Overall, the result of the E169K is a CH848.D949.10.17 envelope capable of eliciting more different types of bnAbs.

The invention contemplates any other design, e.g. stabilized trimer, of the sequences described here in. For non-limiting embodiments of additional stabilized trimers see US2015/0366961, US2020/0002383, US2021/0187091 and US2020/0113997, F14 and/or VT8 designs (US2021/0379177) all of which are incorporated by reference in their entirety.

In certain embodiments the invention provides an envelope comprising 17aa V1 region without N133 and N138 glycosylation, and N301 and N332 glycosylation sites, and further comprising “GDIR” motif (SEQ ID NO: 1), wherein the envelope binds to UCAs of V1V2 Abs and V3 Abs.

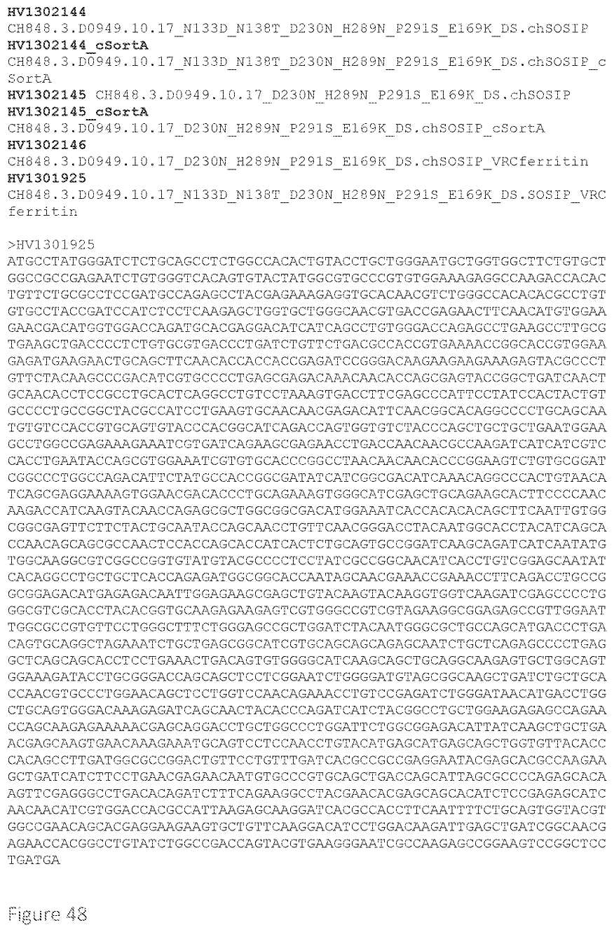

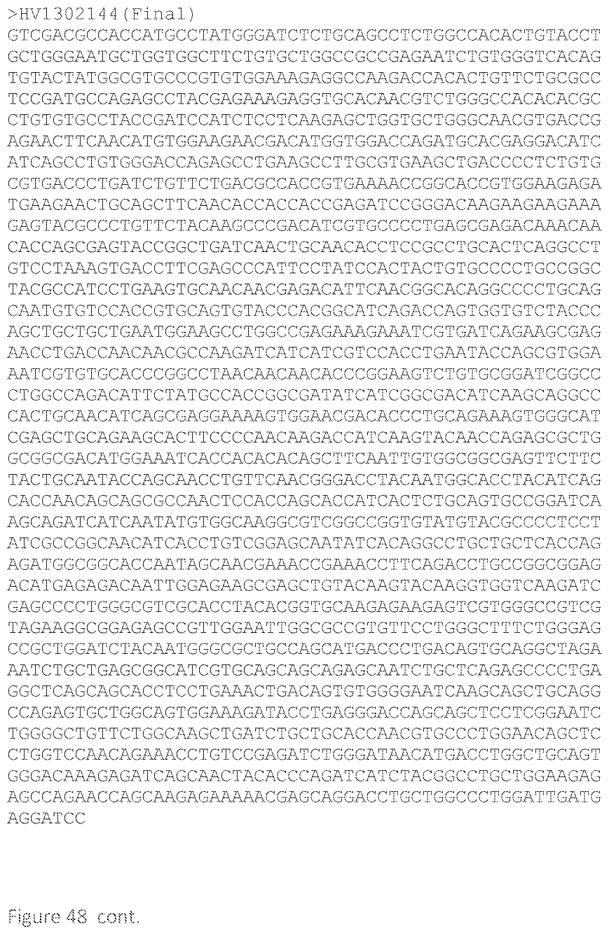

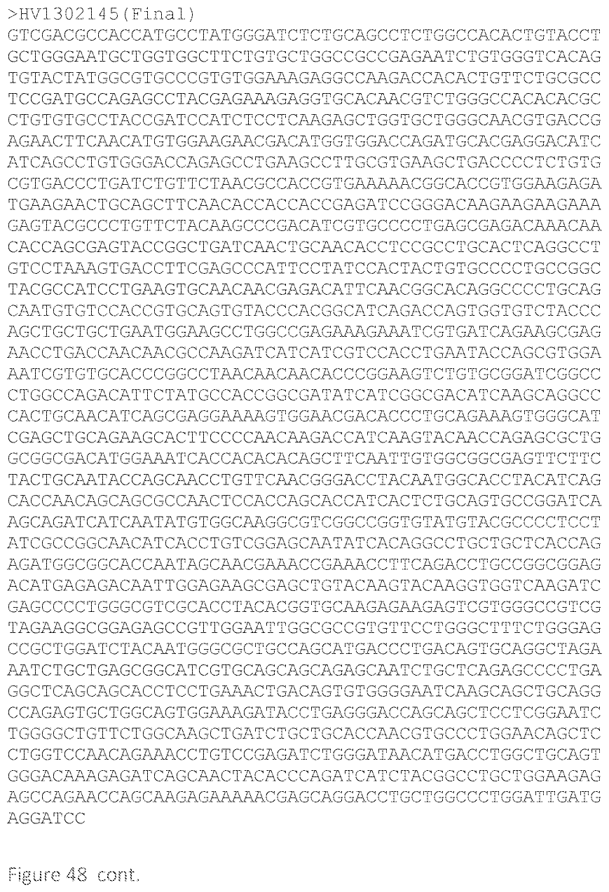

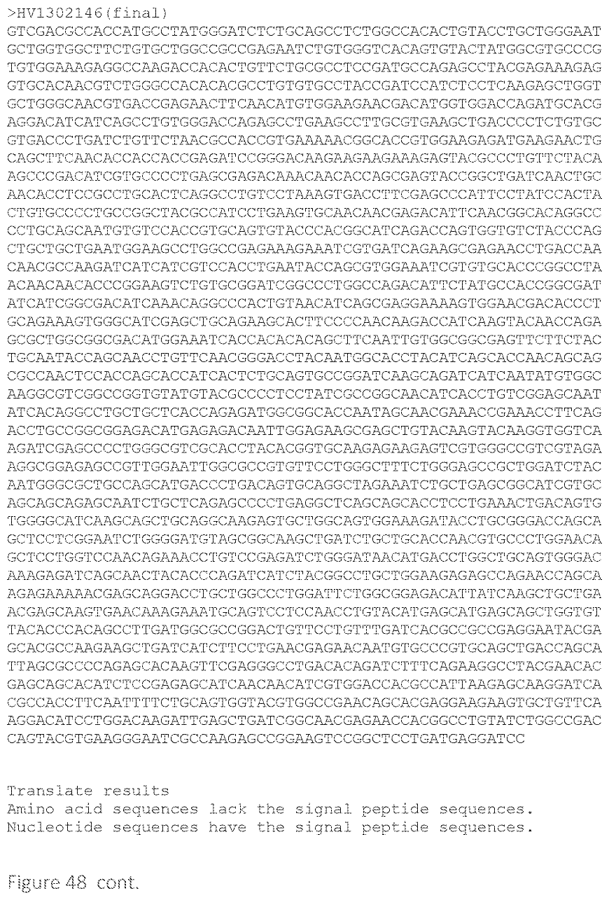

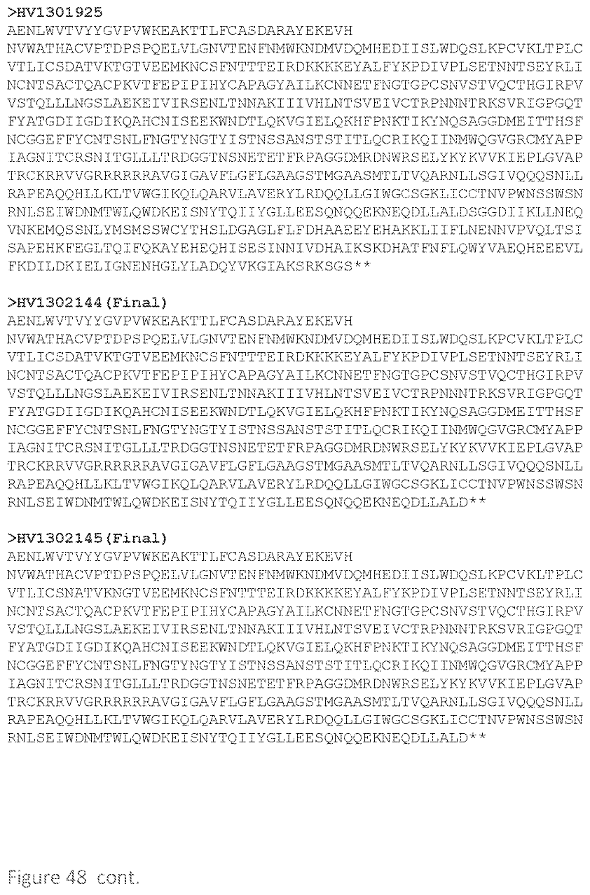

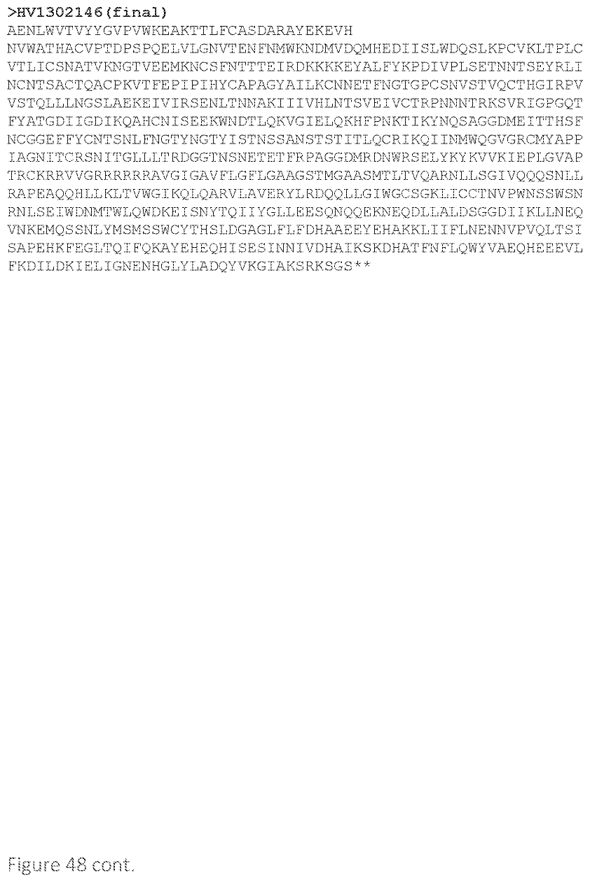

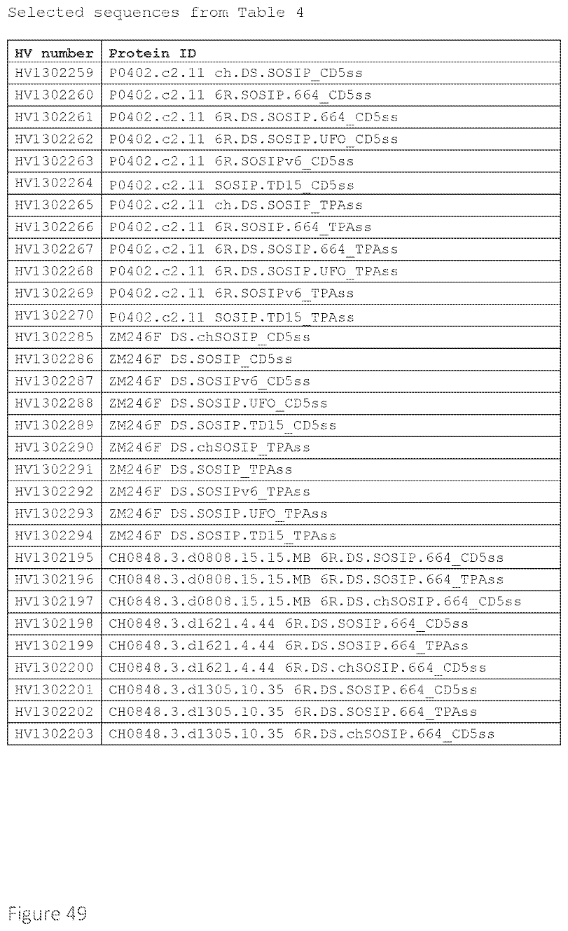

Table 3. Summary of envelope designs for use in prime and boost regimens

| | | Linker | Ferritin for | |
|---|---|---|---|---|
| HV name | Envelope to be used as protein and/or mRNA | sequence | multimerization | FIG. |
| HV1302144 | 10.17 DT protein trimer | no | No | FIG. 48 |
| | CH848.3.D0949.10.17_N133D_N138T_D230N_{—} | | | (SEQ ID |
| | H289N_P291S_E169K_DS.chSOSIP | | | NOS: 170, |
| | | | | 174) |
| HV1302144_{—} | 10.17 DT protein sortaNP | For Sortase | Any Ferritin is | |
| cSortA | CH848.3.D0949.10.17_N133D_N138T_D230N_{—} | conjugation | added via | |
| | H289N_P291S_E169K_DS.chSOSIP_cSortA | | Sortase | |
| | | | conjugation at C- | |
| | | | terminus | |
| HV1301925 | 10.17 DT protein NP | SGG | could be any | FIG. 48 |
| | CH848.3.D0949.10.17_N133D_N138T_D230N_{—} | | suitable ferritin | (SEQ ID |
| | H289N_P291S_E169K_DS.chSOSIP_VRCferritin | | | NOS: 169, |
| | | | | 173) |
| HV1302145 | 10.17 protein trimer | no | | FIG. |
| | CH848.3.D0949.10.17_D230N_H289N_P291S_{—} | | | 48(SEQ |
| | E169K_DS.chSOSIP | | | ID NOS: |
| | | | | 171, 175) |
| HV1302145_{—} | CH848.3.D0949.10.17_D230N_H289N_P291S_{—} | For Sortase | Any Ferritin is | |
| cSortA | E169K_DS.chSOSIP_cSortA | conjugation | added via | |
| | | | Sortase | |
| | | | conjugation at C- | |
| | | | terminus | |
| HV1302146 | CH848.3.D0949.10.17_D230N_H289N_P291S_{—} | SGG | could be any | FIG. 48 |
| | E169K_DS.chSOSIP_VRCferritin | | suitable ferritin | (SEQ ID |
| | | | | NOS: 172, |
| | | | | 176) |

Table 4 Summary of selection of immunogens for induction of neutralizing antibodies.

| | | NEW selection | One embodiment | FIGS. | Envelope protomer |
|---|---|---|---|---|---|
| prime | | CH848.0949.10.17DT | See Table 3, FIG. 48 (e.g., | FIG. 48 (e.g., | Various protomer |
| | | | (SEQ ID NOS: 169, 173, | (SEQ ID | designs, including |
| | | | 170, 174)) | NOS: 169, | without limitation |
| | | | | 173, 170, | various stabilized |
| | | | | 174)) | designs. |
| boost | x | CH848.0949.10.17 | See Table 3, FIG. 48 (e.g., | FIG. 48 (e.g., | Various protomer |
| | | | (SEQ ID NOS: 171, 175, | (SEQ ID | designs, including |
| | | | 172, 176)) | NOS: 171, | without limitation |
| | | | | 175, 172, | various stabilized |
| | | | | 176)) | designs. |
| boost | | CH848.0808.15.15 | CH0848.3.0808.15.15.MB | FIG. 49 (e.g., | CH0848.3.d0808.15.15.MB |
| | | | 6R.DS.SOSIP.664_CD5ss | SEQ ID | 6R.DS.SOSIP.664 |
| | | | | NOs: 199- | With various signal |
| | | | | 201, 230-32 | peptides |
| | | | | | Various protomer |
| | | | | | designs, including |
| | | | | | without limitation |
| | | | | | various stabilized |
| | | | | | designs. |
| boost | x | CH848.0358.80.06 | | FIG. | |
| | | | | 50A(e.g., | |
| | | | | SEQ ID | |
| | | | | NOS: 239- | |
| | | | | 243, 249- | |
| | | | | 253) | |
| boost | x | CH848.1432.5.41 | | FIG. 50A | |
| | | | | (e.g., SEQ | |
| | | | | ID NOS: | |
| | | | | 244-248, | |
| | | | | 254-258) | |
| boost | | CH848.1621.4.44 | CH0848.3.d1621.4.44 | FIG. 49 (e.g., | CH0848.3.d1621.4.44 |
| | | | 6R.DS.SOSIP.664_CD5ss | SEQ ID | 6R.DS.SOSIP.664 |
| | | | | NOS: 202- | With various signal |
| | | | | 204, 233- | peptides |
| | | | | 235) | Various protomer |
| | | | | | designs, including |
| | | | | | without limitation |
| | | | | | various stabilized |
| | | | | | designs |
| boost | | CH848.1305.10.35 | CH0848.3.d1305.10.35 | FIG. 49 (e.g., | CH0848.3.d1305.10.35 |
| | | | 6R.DS.SOSIP.664_CD5ss | SEQ ID | 6R.DS.SOSIP.664 |
| | | | | NOS: 205- | With various signal |
| | | | | 207, 236- | peptides |
| | | | | 238) | Various protomer |
| | | | | | designs, including |
| | | | | | without limitation |
| | | | | | various stabilized |
| | | | | | designs |
| boost | | P0402.c2.11 (G) | | FIG. 49 (e.g., | Various protomer |
| | | | | SEQ ID | designs, including |
| | | | | NOS: 177- | without limitation |
| | | | | 188, 208- | various stabilized |
| | | | | 219) | designs |
| boost | | ZM246F (C) | | FIG. 49 (e.g., | Various protomer |
| | | | | SEQ ID | designs, including |
| | | | | NOS: 189- | without limitation |
| | | | | 198, 220- | various stabilized |
| | | | | 229) | designs |

(x) indicates non-limiting embodiments of boost envelopes described in Table 3.

Throughout the specification, the name CH848.d0949.10.17 DT is interchangeably used as CH848.d0949.10.17.N133D.N138T. Throughout the specification, the name CH848.d0949.10.17 is interchangeably used as CH848.d0949.10.17WT. In certain embodiments, CH848.d0949.10.17DT envelope comprises additional modifications D230N.H289N.P291S.E169K and is referred to as CH848.d0949.10.17 DTe. In certain embodiments, CH848.d0949.10.17 envelope comprises additional modifications D230N.H289N.P291S.E169K and is referred to as CH848.d0949.10.17WTe.

Any suitable signal peptide could be used. In designs comprising ferritin for multimerization, any suitable linker could be used between the envelope sequence and a ferritin sequence.

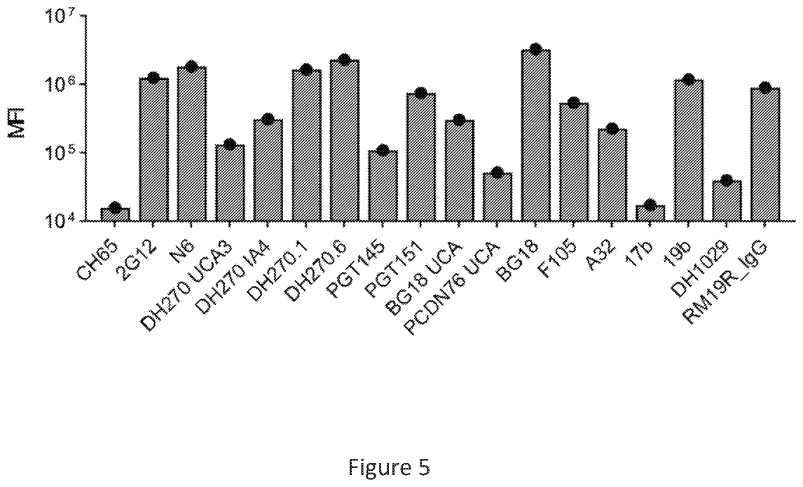

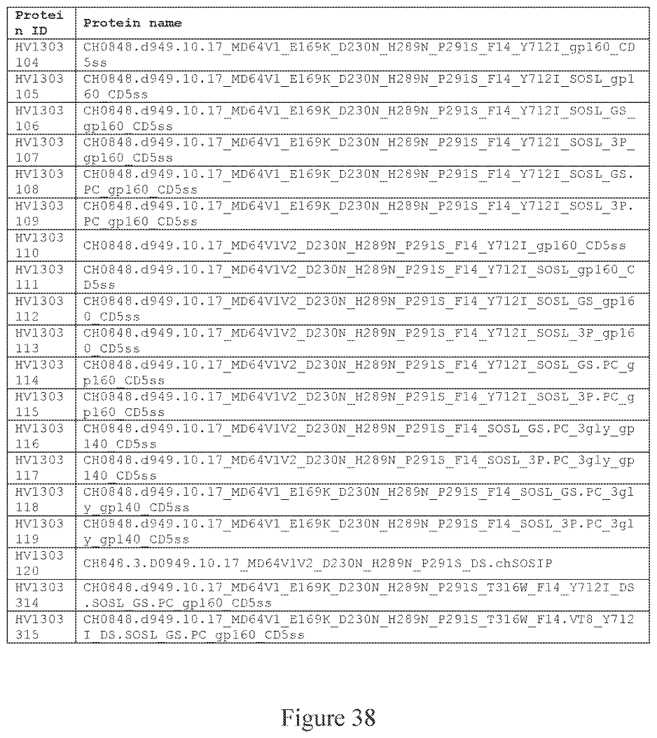

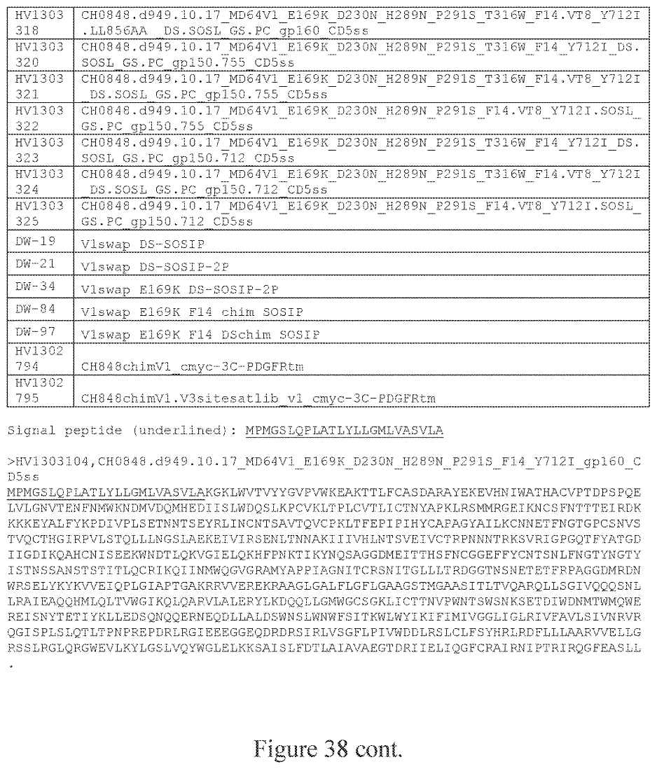

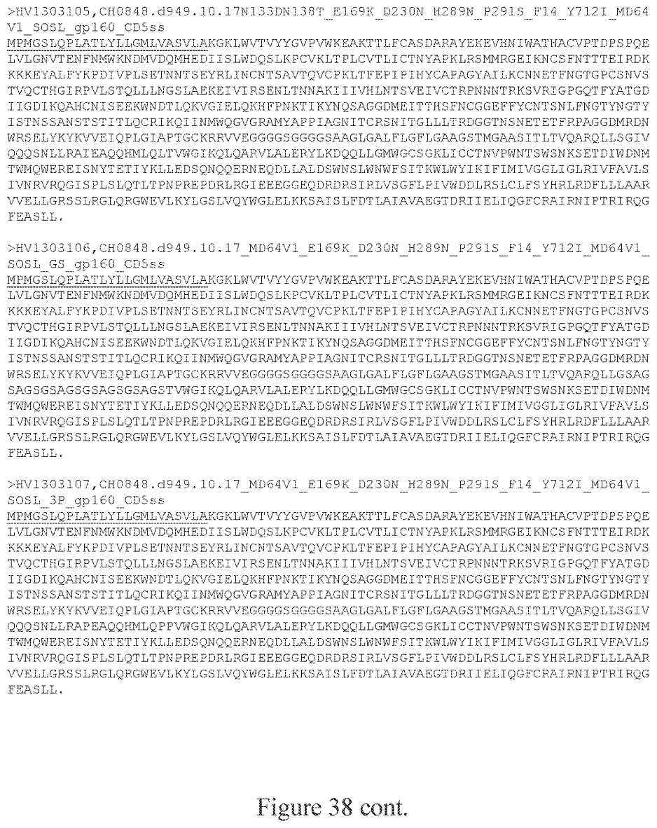

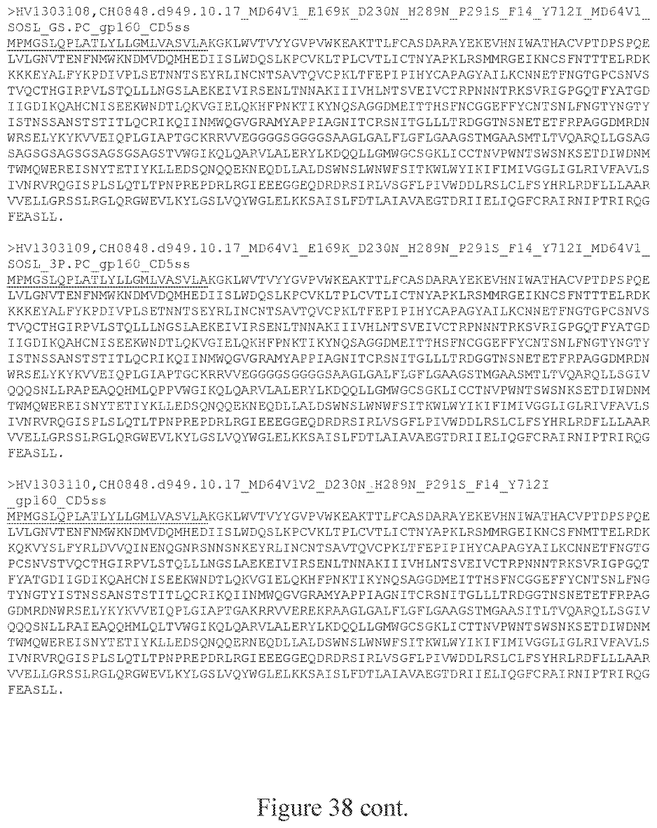

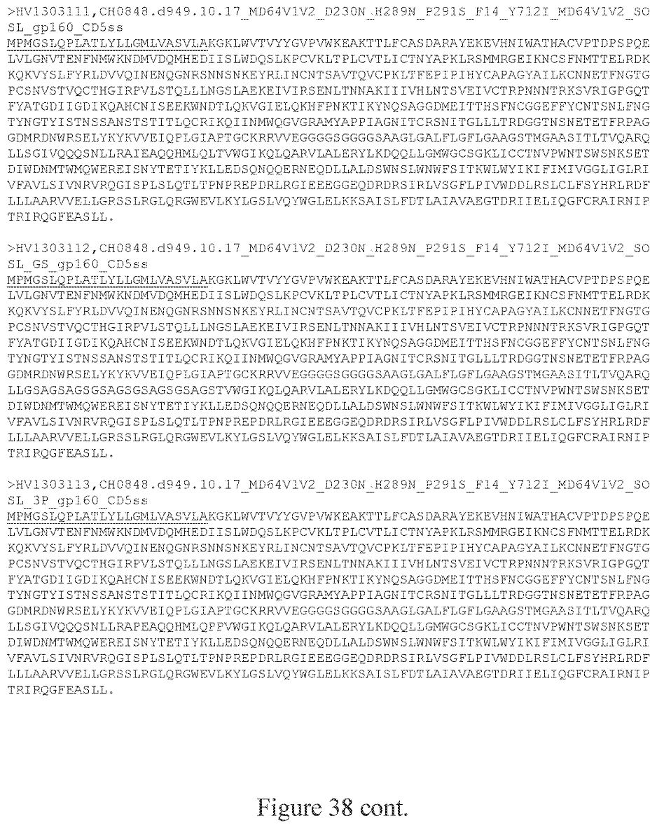

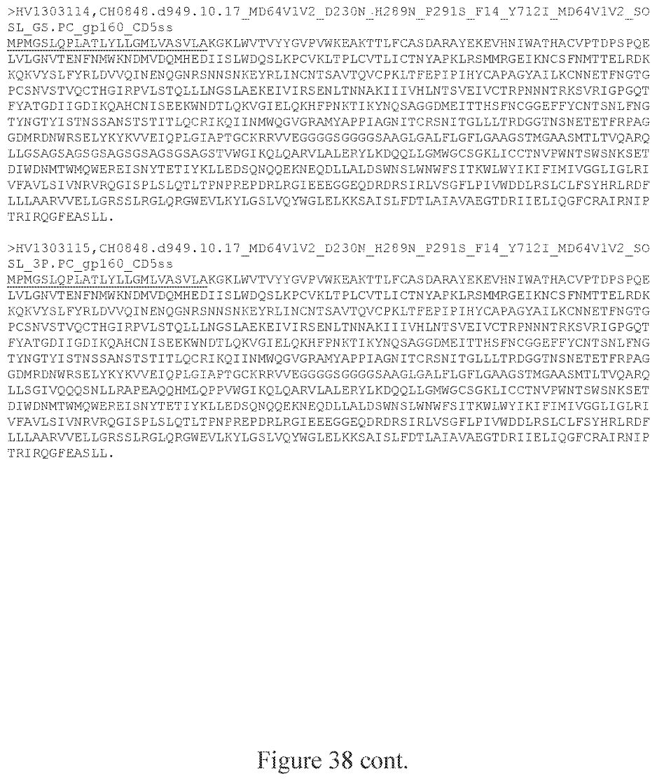

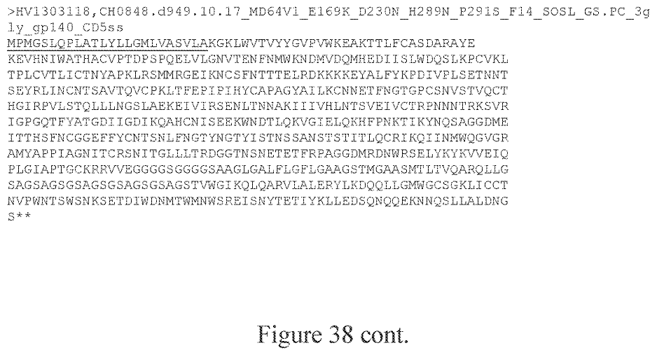

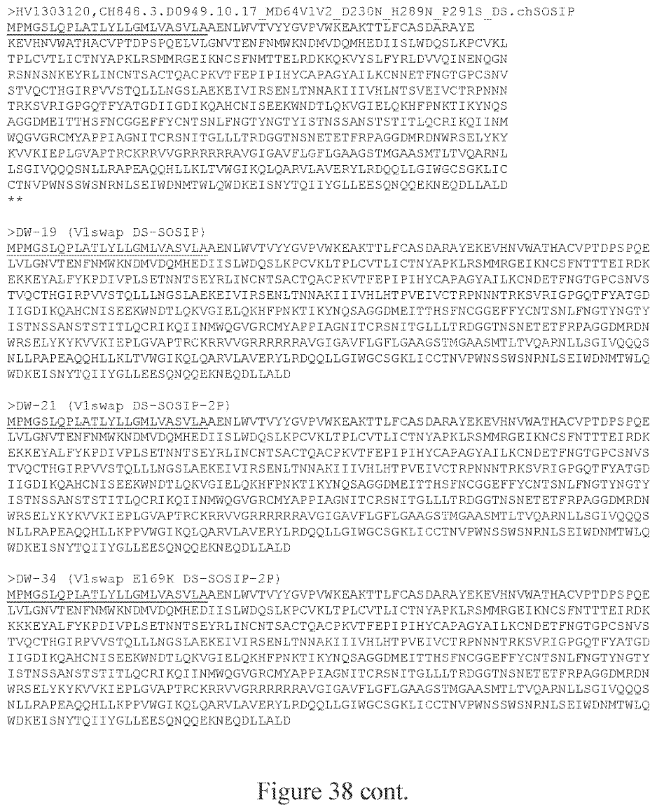

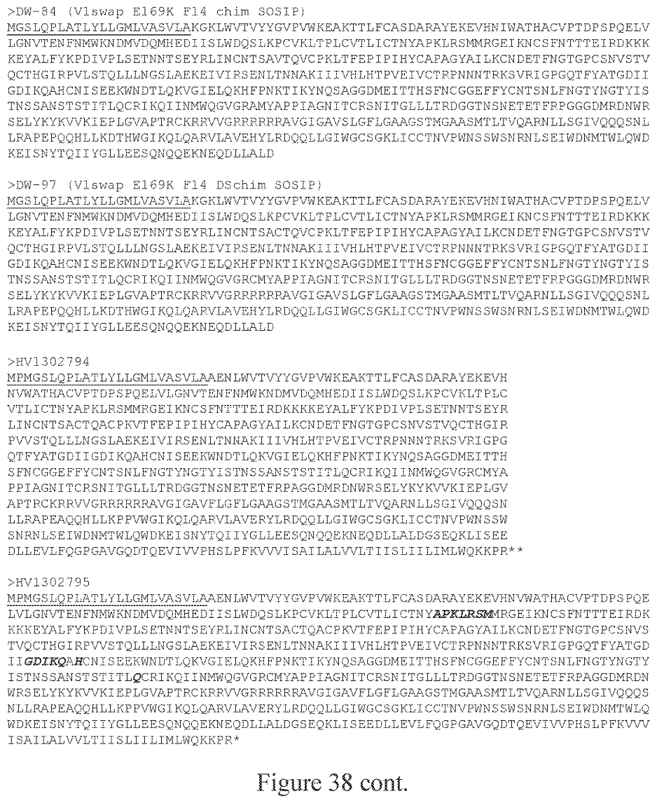

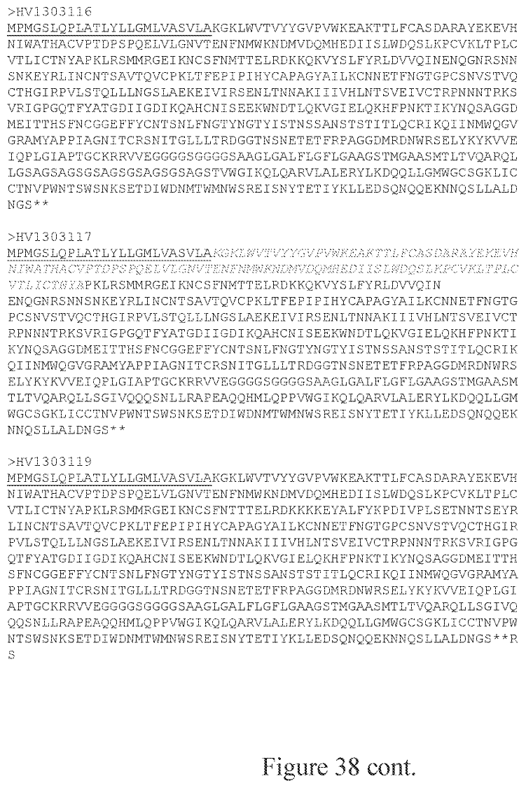

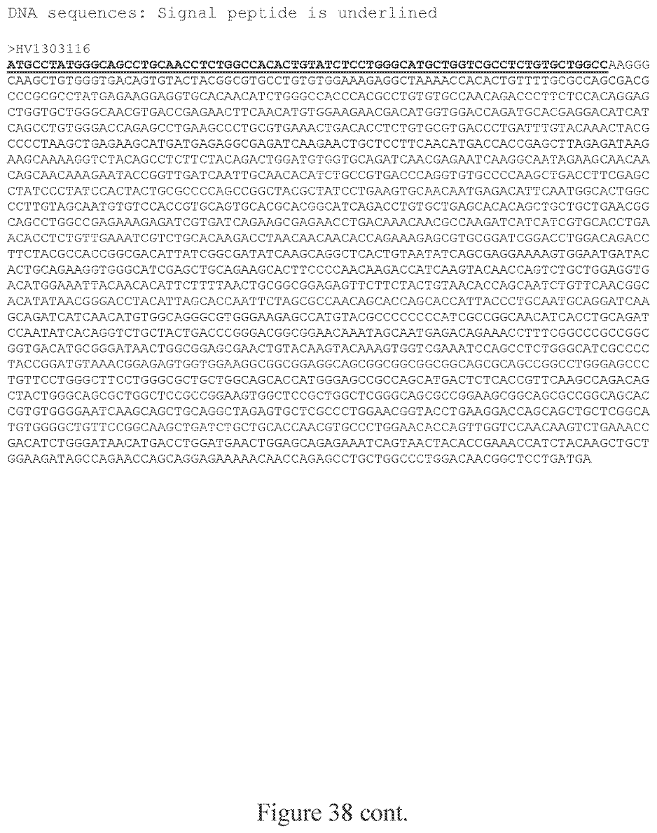

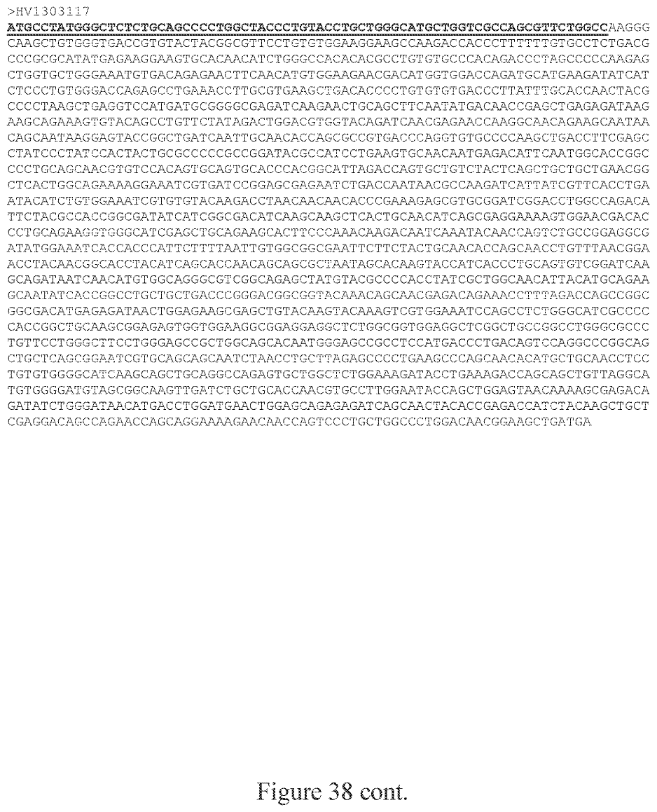

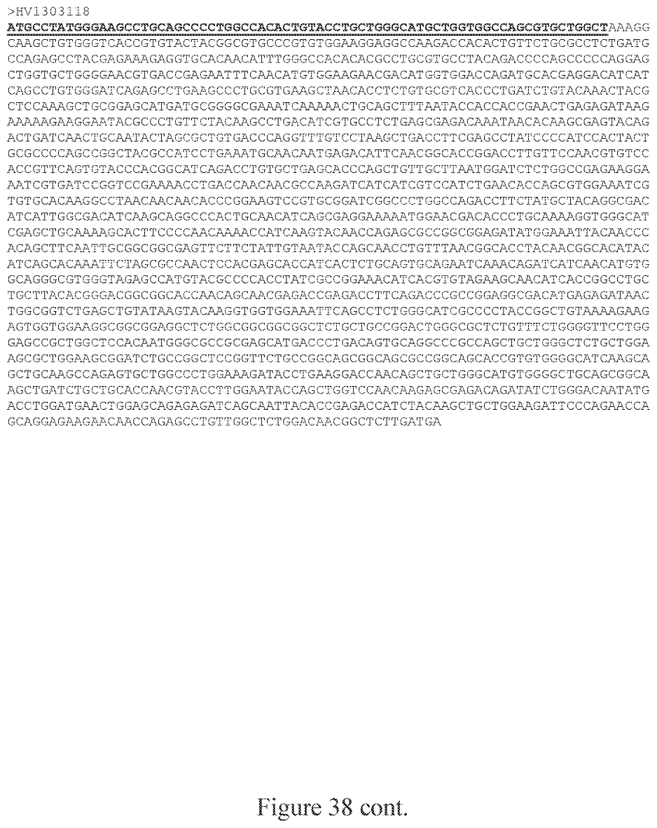

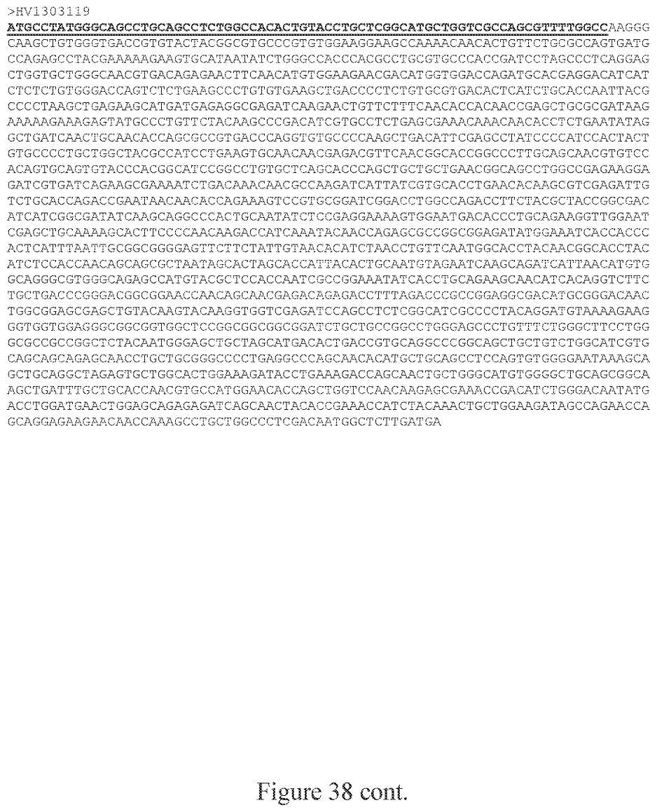

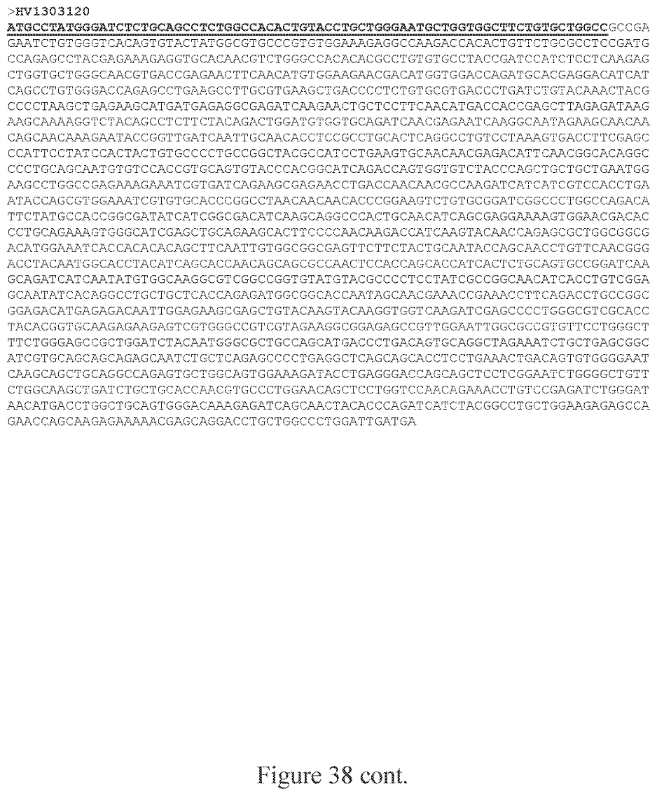

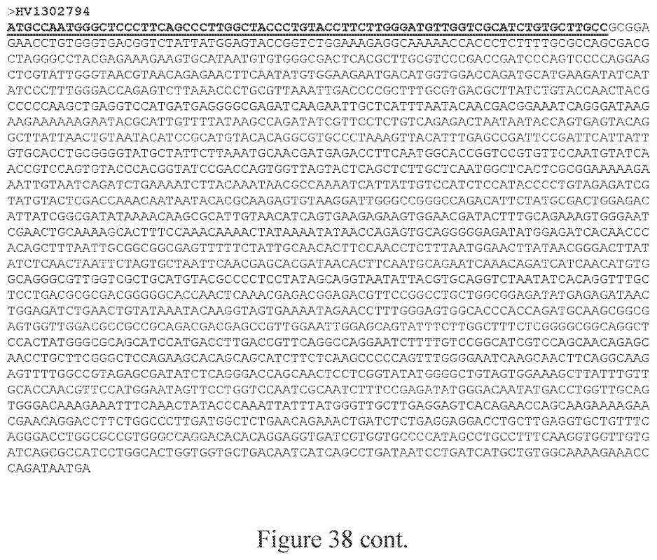

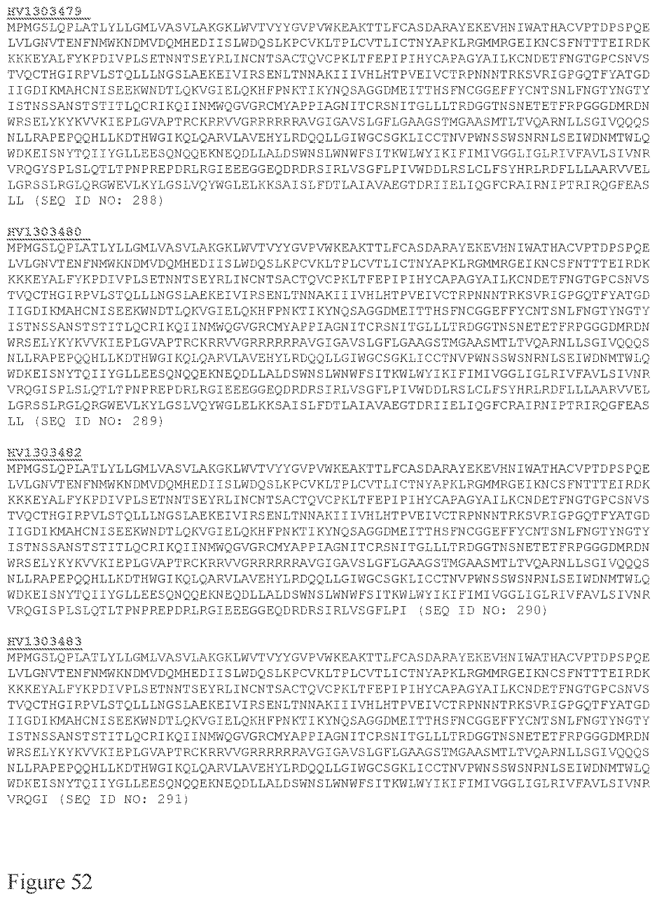

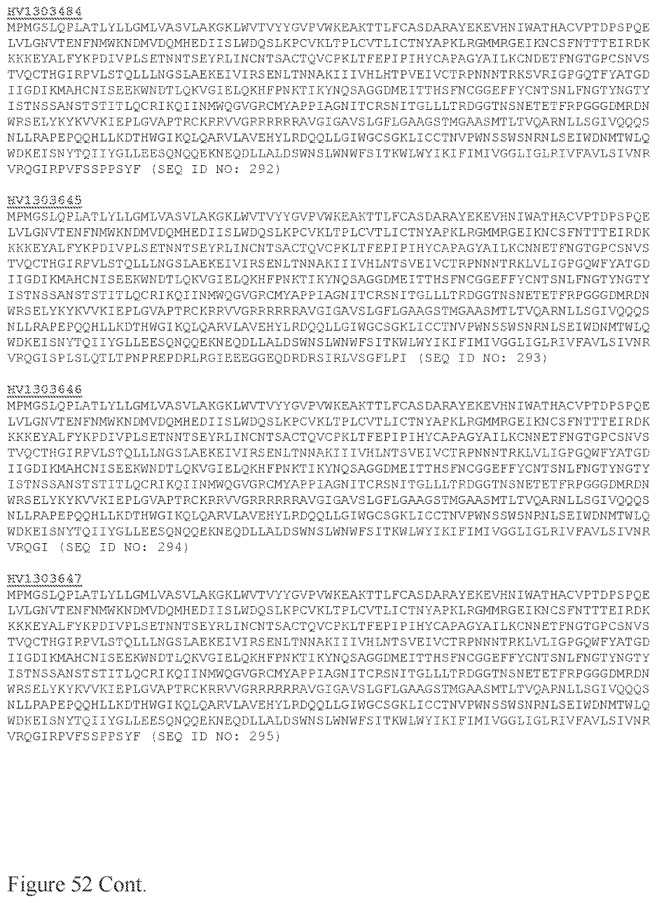

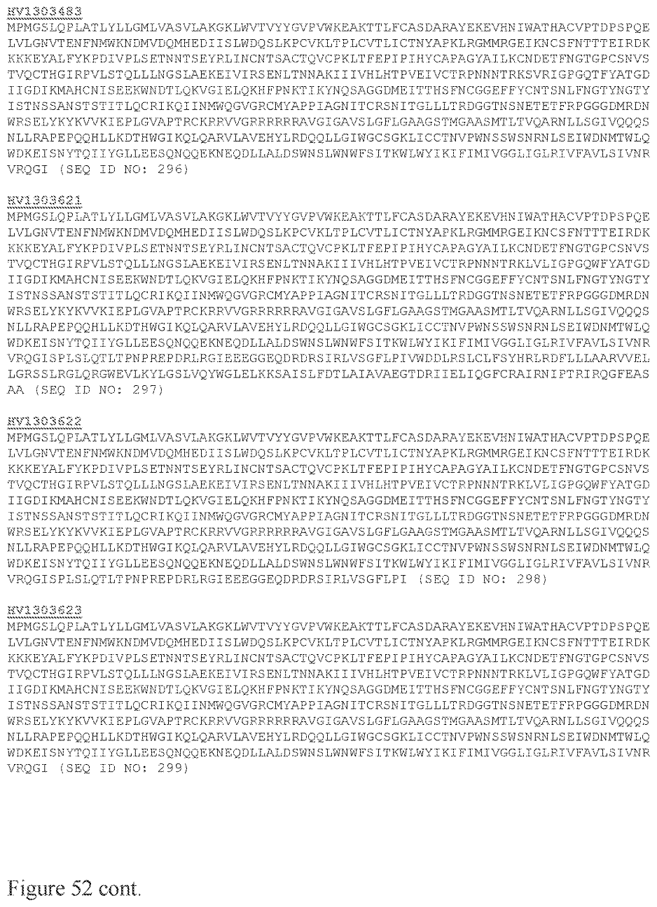

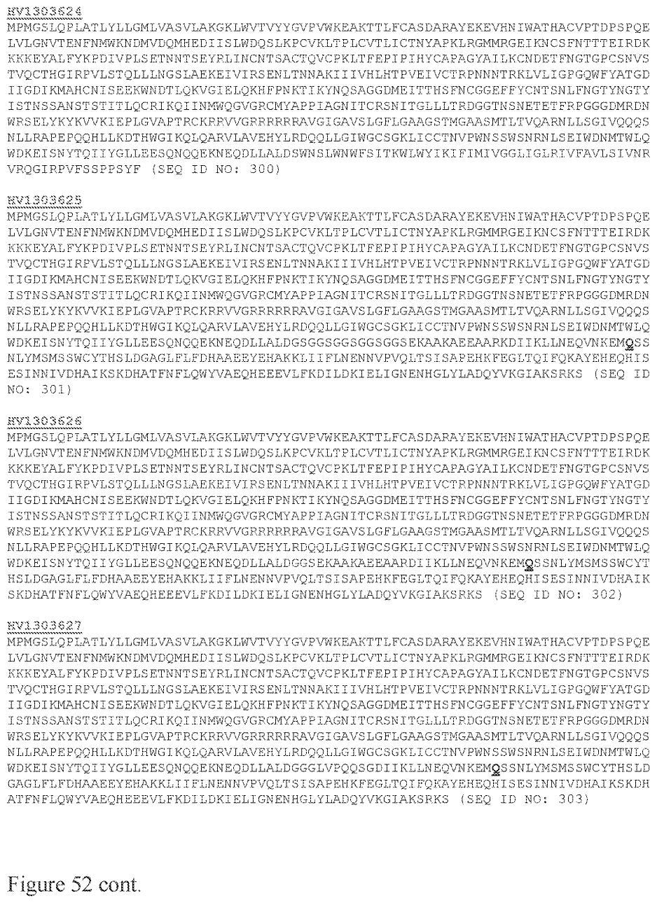

Table 5 shows a summary of amino acid and DNA sequences described herein.

| | | Amino acid, | |
|---|---|---|---|
| Protein ID | Protein name | nucleic acid | FIG. |
| HV1303104 | CH0848.d949.10.17_MD64V1_E169K_D230N_{—} | Aa | FIG. 38 |
| | H289N_P291S_F14_Y712I_gp160_CD5ss | | (SEQ ID NO: |
| | | | 19) |
| HV1303105 | CH0848.d949.10.17_MD64V1_E169K_D230N_{—} | Aa | FIG. 38 |
| | H289N_P291S_F14_Y712I_SOSL_gp160_CD5ss | | (SEQ ID NO: |
| | | | 20) |
| HV1303106 | CH0848.d949.10.17_MD64V1_E169K_D230N_{—} | Aa | FIG. 38 |
| | H289N_P291S_F14_Y712I_SOSL_GS_gp160_CD5ss | | (SEQ ID NO: |
| | | | 21) |
| HV1303107 | CH0848.d949.10.17_MD64V1_E169K_D230N_{—} | Aa | FIG. 38 |
| | H289N_P291S_F14_Y712I_SOSL_3P_gp160_CD5ss | | (SEQ ID NO: |
| | | | 22) |
| HV1303108 | CH0848.d949.10.17_MD64V1_E169K_D230N_{—} | Aa | FIG. 38 |
| | H289N_P291S_F14_Y712I_SOSL_GS.PC_gp160_{—} | | (SEQ ID NO: |
| | CD5ss | | 23) |
| HV1303109 | CH0848.d949.10.17_MD64V1_E169K_D230N_{—} | Aa | FIG. 38 |
| | H289N_P291S_F14_Y712I_SOSL_3P.PC_gp160_{—} | | (SEQ ID NO: |
| | CD5ss | | 24) |
| HV1303110 | CH0848.d949.10.17_MD64V1V2_D230N_H289N_{—} | Aa | FIG. 38 |
| | P291S_F14_Y712I_gp160_CD5ss | | (SEQ ID NO: |
| | | | 25) |
| HV1303111 | CH0848.d949.10.17_MD64V1V2_D230N_H289N_{—} | Aa | FIG. 38 |
| | P291S_F14_Y712I_SOSL_gp160_CD5ss | | (SEQ ID NO: |
| | | | 26) |
| HV1303112 | CH0848.d949.10.17_MD64V1V2_D230N_H289N_{—} | Aa | FIG. 38 |
| | P291S_F14_Y712I_SOSL_GS_gp160_CD5ss | | (SEQ ID NO: |
| | | | 27) |
| HV1303113 | CH0848.d949.10.17_MD64V1V2_D230N_H289N_{—} | Aa | FIG. 38 |
| | P291S_F14_Y712I_SOSL_3P_gp160_CD5ss | | (SEQ ID NO: |
| | | | 28) |
| HV1303114 | CH0848.d949.10.17_MD64V1V2_D230N_H289N_{—} | Aa | FIG. 38 |
| | P291S_F14_Y712I_SOSL_GS.PC_gp160_CD5ss | | (SEQ ID NO: |
| | | | 29) |
| HV1303115 | CH0848.d949.10.17_MD64V1V2_D230N_H289N_{—} | Aa | FIG. 38 |
| | P291S_F14_Y712I_SOSL_3P.PC_gp160_CD5ss | | (SEQ ID NO: |
| | | | 30 |
| HV1303116 | CH0848.d949.10.17_MD64V1V2_D230N_H289N_{—} | Aa | FIG. 38 |
| | P291S_F14_SOSL_GS.PC_3gly_gp140_CD5ss | | (SEQ ID NO: |
| | | | 43) |
| | | Nt (DNA) | FIG. 38 |
| | | | (SEQ ID NO: |
| | | | 46) |
| HV1303117 | CH0848.d949.10.17_MD64V1V2_D230N_H289N_{—} | Aa | FIG. 38 |
| | P291S_F14_SOSL_3P.PC_3gly_gp140_CD5ss | | (SEQ ID NO: |
| | | | 44) |
| | | Nt (DNA) | FIG. 38 |
| | | | (SEQ ID NO: |
| | | | 47) |
| HV1303118 | CH0848.d949.10.17_MD64V1_E169K_D230N_H289N_{—} | Aa | FIG. 38 |
| | P291S_F14_SOSL_GS.PC_3gly_gp140_CD5ss | | (SEQ ID NO: |
| | | | 33) |
| | | Nt (DNA) | FIG. 38 |
| | | | (SEQ ID NO: |
| | | | 48) |
| HV1303119 | CH0848.d949.10.17_MD64V1_E169K_D230N_H289N_{—} | Aa | FIG. 38 |
| | P291S_F14_SOSL_3P.PC_3gly_gp140_CD5ss | | (SEQ ID NO: |
| | | | 45) |
| | | Nt (DNA) | FIG. 38 |
| | | | (SEQ ID NO: |
| | | | 49) |
| HV1303120 | CH848.3.D0949.10.17_MD64V1V2_D230N_H289N_{—} | Aa | FIG. 38 |
| | P291S_DS.chSOSIP | | (SEQ ID NO: |
| | | | 35) |
| | | Nt (DNA) | FIG. 38 |
| | | | (SEQ ID NO: |
| | | | 50) |
| HV1303314 | CH0848.d949.10.17_MD64V1_E169K_D230N_{—} | Aa | FIG. 39 |
| | H289N_P291S_T316W_F14_Y712I_DS.SOSL_GS.PC_{—} | | (SEQ ID NO: |
| | gp160_CD5ss | | 52) |
| HV1303315 | CH0848.d949.10.17_MD64V1_E169K_D230N_{—} | Aa | FIG. 39 |
| | H289N_P291S_T316W_F14.VT8_Y712I_DS.SOSL_{—} | | (SEQ ID NO: |
| | GS.PC_gp160_CD5ss | | 53) |
| HV1303316 | CH0848.d949.10.17_MD64V1_E169K_D230N_{—} | Aa | FIG. 39 |
| | H289N_P291S_F14.VT8_Y712I.SOSL_GS.PC_gp160_{—} | | (SEQ ID NO: |
| | CD5ss | | 54; |
| HV1303317 | CH0848.d949.10.17_MD64V1_E169K_D230N_{—} | Aa | FIG. 39 |
| | H289N_P291S_T316W_F14_Y712I.LL856AA_DS.SOSL_{—} | | (SEQ ID NO: |
| | GS.PC_gp160_CD5ss | | 55) |
| HV1303318 | CH0848.d949.10.17_MD64V1_E169K_D230N_H289N_{—} | Aa | FIG. 39 |
| | P291S_T316W_F14.VT8_Y712I.LL856AA_{—} | | (SEQ ID NO: |
| | DS.SOSL_GS.PC_gp160_CD5ss | | 56) |
| HV1303319 | CH0848.d949.10.17_MD64V1_E169K_D230N_H289N_{—} | Aa | FIG. 39 |
| | P291S_F14.VT8_Y712I.LL856AA.SOSL_GS.PC_{—} | | (SEQ ID NO: |
| | gp160_CD5ss | | 57) |
| HV1303320 | CH0848.d949.10.17_MD64V1_E169K_D230N_{—} | Aa | FIG. 39 |
| | H289N_P291S_T316W_F14_Y712I_DS.SOSL_GS.PC_{—} | | (SEQ ID NO: |
| | gp150.755_CD5ss | | 58) |
| HV1303321 | CH0848.d949.10.17_MD64V1_E169K_D230N_H289N_{—} | Aa | FIG. 39 |
| | P291S_T316W_F14.VT8_Y712I_DS.SOSL_GS.PC_{—} | | (SEQ ID NO: |
| | gp150.755_CD5ss | | 59) |
| HV1303322 | CH0848.d949.10.17_MD64V1_E169K_D230N_H289N_{—} | Aa | FIG. 39 |
| | P291S_F14.VT8_Y712I.SOSL_GS.PC_gp150.755_{—} | | (SEQ ID NO: |
| | CD5ss | | 60) |
| HV1303323 | CH0848.d949.10.17_MD64V1_E169K_D230N_H289N_{—} | Aa | FIG. 39 |
| | P291S_T316W_F14_Y712I_DS.SOSL_GS.PC_{—} | | (SEQ ID NO: |
| | gp150.712_CD5ss | | 61) |
| HV1303324 | CH0848.d949.10.17_MD64V1_E169K_D230N_H289N_{—} | Aa | FIG. 40 |
| | P291S_T316W_F14.VT8_Y712I_DS.SOSL_{—} | | (SEQ ID NO: |
| | GS.PC_gp150.712_CD5ss | | 62) |
| HV1303325 | CH0848.d949.10.17_MD64V1_E169K_D230N_H289N_{—} | Aa | FIG. 40 |
| | P291S_F14.VT8_Y712I.SOSL_GS.PC_gp150.712_{—} | | (SEQ ID NO: |
| | CD5ss | | 63) |
| DW-19 | V1swap DS-SOSIP | Aa | FIG. 38 |
| | | | (SEQ ID NO: |
| | | | 36 |
| DW-21 | V1swap DS-SOSIP-2P | Aa | FIG. 38 |
| | | | (SEQ ID NO: |
| | | | 37) |
| DW-34 | V1swap E169K DS-SOSIP-2P | Aa | FIG. 38 |
| | | | (SEQ ID NO: |
| | | | 38) |
| DW-84 | V1swap E169K F14 chim SOSIP | Aa | FIG. 38 |
| | | | (SEQ ID NO: |
| | | | 39) |
| DW-97 | V1swap E169K F14 DSchim SOSIP | Aa | FIG. 38 |
| | | | (SEQ ID NO: |
| | | | 40) |
| HV1302794 | CH848chimV1_cmyc-3C-PDGFRtm | Aa | FIG. 38 |
| | | | (SEQ ID NO: |
| | | | 41) |
| | | Nt (DNA) | FIG. 38 |
| | | | (SEQ ID NO: |
| | | | 51) |
| HV1302795 | CH848chimV1.V3sitesatlib_v1_cmyc-3C- | Aa | FIG. 38 |
| | PDGFRtm | | (SEQ ID NO: |
| | | | 42) |
| HV1303479 | CH0848.d949.10.17_V1swap_E169K_S148G_{—} | Aa | FIG. 52 |
| | Q328M_F14_DS_SOSIP_MD2_MD33_gp160_CD5ss | | (SEQ ID NO: |
| | | | 288) |
| HV1303480 | CH0848.d949.10.17_V1swap_E169K_S148G_Q328M_{—} | Aa | FIG. 52 |
| | F14_DS_SOSIP_MD2_MD33_Y712I_gp160_{—} | | (SEQ ID NO: |
| | CD5ss | | 289) |
| HV1303482 | CH0848.d949.10.17_V1swap_E169K_S148G_Q328M_{—} | Aa | FIG. 5 |
| | F14_DS_SOSIP_MD2_MD33_Y712I_{—} | | (SEQ ID NO: |
| | gp150.755_CD5ss | | 290) |
| HV1303483 | CH0848.d949.10.17_V1swap_E169K_S148G_Q328M_{—} | Aa | FIG. 52 |
| | F14_DS_SOSIP_MD2_MD33_Y712I_{—} | | (SEQ ID |
| | gp150.712_CD5ss | | NOs: 291 |
| | | | 296) |
| HV1303484 | CH0848.d949.10.17_V1swap_E169K_S148G_Q328M_{—} | Aa | FIG. 52 |
| | F14_DS_SOSIP_MD2_MD33_Y712I_gp150.TM1_{—} | | (SEQ ID NO: |
| | CD5ss | | 292) |
| HV1303645 | CH0848.d949.10.17_V1swap_E169K_S148G_Q328M_{—} | Aa | FIG. 52 |
| | D230N_H289N_P291S_S306L_R308L_T316W_{—} | | (SEQ ID NO: |
| | F14_DS_SOSIP_MD2_MD33_Y712I_gp150.755_{—} | | 293) |
| | CD5ss | | |
| HV1303646 | CH0848.d949.10.17_V1swap_E169K_S148G_Q328M_{—} | Aa | FIG. 52 |
| | D230N_H289N_P291S_S306L_R308L_T316W_{—} | | (SEQ ID NO: |
| | F14_DS_SOSIP_MD2_MD33_Y712I_gp150.712_{—} | | 294) |
| | CD5ss | | |
| HV1303647 | CH0848.d949.10.17_V1swap_E169K_S148G_Q328M_{—} | Aa | FIG. 52 |
| | D230N_H289N_P291S_S306L_R308L_T316W_{—} | | (SEQ ID NO: |
| | F14_DS_SOSIP_MD2_MD33_Y712I_gp150.TM1_{—} | | 295) |
| | CD5ss | | |
| HV1303621 | CH0848.d949.10.17_V1swap_E169K_S148G_Q328M_{—} | Aa | FIG. 52 |
| | S306L_R308L_T316W_F14_DS_SOSIP_MD2_{—} | | (SEQ ID NO: |
| | MD33_Y712I.LL856AA_qp160_CD5ss | | 297) |
| HV1303622 | CH0848.d949.10.17_V1swap_E169K_S148G_Q328M_{—} | Aa | FIG. 52 |
| | S306L_R308L_T316W_F14_DS_SOSIP_MD2_{—} | | (SEQ ID NO: |
| | MD33_Y712I_ gp150.755_CD5ss | | 298) |
| HV1303623 | CH0848.d949.10.17_V1swap_E169K_S148G_Q328M_{—} | Aa | FIG. 52 |
| | S306L_R308L_T316W_F14_DS_SOSIP_MD2_{—} | | (SEQ ID NO: |
| | MD33_Y712I_ gp150.712_CD5ss | | 299) |
| HV1303624 | CH0848.d949.10.17_V1swap_E169K_S148G_Q328M_{—} | Aa | FIG. 52 |
| | S306L_R308L_T316W_F14_DS_SOSIP_MD2_{—} | | (SEQ ID NO: |
| | MD33_Y712I_gp150.TM1_CD5ss | | 300 |
| HV1303625 | CH0848.d949.10.17_V1swap_E169K_S148G_Q328M_{—} | Aa | FIG. 52 |
| | S306L_R308L_T316W_F14_DS_SOSIP_MD2 | | (SEQ ID NO: |
| | MD33_25lnGSERK_VRCFerritin | | 301 |
| HV1303626 | CH0848.d949.10.17_V1swap_E169K_S148G_Q328M_{—} | Aa | FIG. 52 |
| | S306L_R308L_T316W_F14_DS_SOSIP_MD2_{—} | | (SEQ ID NO: |
| | MD33_14lnGSERK_VRCFerritin | | 302) |
| HV1303627 | CH0848.d949.10.17_V1swap_E169K_S148G_Q328M_{—} | Aa | FIG. 52 |
| | S306L_R308L_T316W_F14_DS_SOSIP_MD2_{—} | | (SEQ ID NO: |
| | MD33_10lnQQ_VRCFerritin | | 303) |

Table 6 shows a summary of mRNA sequences described herein.

| | | Amino acid, | |
|---|---|---|---|
| Protein ID | Protein name | nucleic acid | FIG. |
| HV1303104 | CH0848.d949.10.17_MD64V1_E169K_D230N_{—} | Nt(e.g. for | FIG. 40 |
| | H289N_P291S_F14_Y712I_gp160_CD5ss | production | (SEQ ID |
| | | as mRNA) | NO: 64) |
| HV1303105 | CH0848.d949.10.17_MD64V1_E169K_D230N_{—} | Nt(e.g. for | FIG. 40 |
| | H289N_P291S_F14_Y712I_SOSL_gp160_CD5ss | production | (SEQ ID |
| | | as mRNA) | NO: 65) |
| HV1303106 | CH0848.d949.10.17_MD64V1_E169K_D230N_{—} | Nt(e.g. for | FIG. 40 |
| | H289N_P291S_F14_Y712I_SOSL_GS_gp160_CD5ss | production | (SEQ ID |
| | | as mRNA) | NO: 66) |
| V1303107 | CH0848.d949.10.17_MD64V1_E169K_D230N_{—} | Nt(e.g. for | FIG. 40 |
| | H289N_P291S_F14_Y712I_SOSL_3P_gp160_CD5ss | production | (SEQ ID |
| | | as mRNA) | NO: 67) |
| HV1303108 | CH0848.d949.10.17_MD64V1_E169K_D230N_{—} | Nt(e.g. for | FIG. 40 |
| | H289N_P291S_F14_Y712I_SOSL_GS.PC_gp160_{—} | production | (SEQ ID |
| | CD5ss | as mRNA) | NO: 68) |
| HV1303109 | CH0848.d949.10.17_MD64V1_E169K_D230N_{—} | Nt(e.g. for | FIG. 40 |
| | H289N_P291S_F14_Y712I_SOSL_3P.PC_gp160_{—} | production | (SEQ ID |
| | CD5ss | as mRNA) | NO: 69) |
| HV1303110 | CH0848.d949.10.17_MD64V1V2_D230N_H289N_{—} | Nt(e.g. for | FIG. 40 |
| | P291S_F14_Y712I_gp160_CD5ss | production | (SEQ ID |
| | | as mRNA) | NO: 70 |
| HV1303111 | CH0848.d949.10.17_MD64V1V2_D230N_H289N_{—} | Nt(e.g. for | FIG. 40 |
| | P291S_F14_Y712I_SOSL_gp160_CD5ss | production | (SEQ ID |
| | | as mRNA) | NO: 71) |
| HV1303112 | CH0848.d949.10.17_MD64V1V2_D230N_H289N_{—} | Nt(e.g. for | FIG. 40 |
| | P291S_F14_Y712I_SOSL_GS_gp160_CD5ss | production | (SEQ ID |
| | | as mRNA) | NO: 72) |
| HV1303113 | CH0848.d949.10.17_MD64V1V2_D230N_H289N_{—} | Nt(e.g. for | FIG. 40 |
| | P291S_F14_Y712I_SOSL_3P_gp160_CD5ss | production | (SEQ ID |
| | | as mRNA) | NO: 73) |
| HV1303114 | CH0848.d949.10.17_MD64V1V2_D230N_H289N_{—} | Nt(e.g. for | FIG. 40 |
| | P291S_F14_Y712I_SOSL_GS.PC_gp160_CD5ss | production | (SEQ ID |
| | | as mRNA) | NO: 74) |
| HV1303115 | CH0848.d949.10.17_MD64V1V2_D230N_H289N_{—} | Nt(e.g. for | FIG. 40 |
| | P291S_F14_Y712I_SOSL_3P.PC_gp160_CD5ss | production | (SEQ ID |
| | | as mRNA) | NO: 75) |
| HV1303118 | CH0848.d949.10.17_MD64V1_E169K_D230N_{—} | Nt(e.g. for | FIG. 40 |
| | H289N_P291S_F14_SOSL_GS.PC_3gly_gp140_CD5ss | production | (SEQ ID |
| | | as mRNA) | NO: 93) |
| HV1303120 | CH848.3.D0949.10.17_MD64V1V2_D230N_H289N_{—} | Nt(e.g. for | FIG. 40 |
| | P291S_DS.chSOSIP | production | (SEQ ID |
| | | as mRNA) | NO: 94) |
| HV1303314 | CH0848.d949.10.17_MD64V1_E169K_D230N_{—} | Nt(e.g. for | FIGS. 40, |
| | H289N_P291S_T316W_F14_Y712I_DS.SOSL_{—} | production | 58 (SEQ |
| | GS.PC_gp160_CD5ss | as mRNA) | ID NOS: |
| | | | 81, 320) |
| HV1303315 | CH0848.d949.10.17_MD64V1_E169K_D230N_{—} | Nt(e.g. for | FIGS. 40, |
| | H289N_P291S_T316W_F14.VT8_Y712I_DS.SOSL_{—} | production | 58 (SEQ |
| | GS.PC_gp160_CD5ss | as mRNA) | ID NO: 82, |
| | | | 321) |
| HV1303316 | CH0848.d949.10.17_MD64V1_E169K_D230N_{—} | Nt(e.g. for | FIGS. 40, |
| | H289N_P291S_F14.VT8_Y712I.SOSL_GS.PC_{—} | production | 58 (SEQ |
| | gp160_CD5ss | as mRNA) | ID NO: 83, |
| | | | 322) |
| HV1303317 | CH0848.d949.10.17_MD64V1_E169K_D230N_{—} | Nt(e.g. for | FIG. 40 |
| | H289N_P291S_T316W_F14_Y712I.LL856AA_{—} | production | (SEQ ID |
| | DS.SOSL_GS.PC_gp160_CD5ss | as mRNA) | NO: 84) |
| HV1303318 | CH0848.d949.10.17_MD64V1_E169K_D230N_{—} | Nt(e.g. for | FIGS. 40, |
| | H289N_P291S_T316W_F14.VT8_Y712I.LL856AA_{—} | production | 58 (SEQ |
| | DS.SOSL_GS.PC_gp160_CD5ss | as mRNA) | ID NO: 85, |
| | | | 323) |
| HV1303319 | CH0848.d949.10.17_MD64V1_E169K_D230N_{—} | Nt(e.g. for | FIGS. 40, |
| | H289N_P291S_F14.VT8_Y712I.LL856AA.SOSL_{—} | production | 58 (SEQ |
| | GS.PC_gp160_CD5ss | as mRNA) | ID NO: 86, |
| | | | 324) |
| HV1303320 | CH0848.d949.10.17_MD64V1_E169K_D230N_{—} | Nt(e.g. for | FIGS. 40, |
| | H289N_P291S_T316W_F14_Y712I_DS.SOSL_GS.PC_{—} | production | 58 (SEQ |
| | gp150.755_CD5ss | as mRNA) | ID NO: 87, |
| | | | 325) |
| HV1303321 | CH0848.d949.10.17_MD64V1_E169K_D230N_{—} | Nt(e.g. for | FIGS. 40, |
| | H289N_P291S_T316W_F14.VT8_Y712I_DS.SOSL_{—} | production | 58 (SEQ |
| | GS.PC_gp150.755_CD5ss | as mRNA) | ID NO: 88, |
| | | | 326) |
| HV1303322 | CH0848.d949.10.17_MD64V1_E169K_D230N_{—} | Nt(e.g. for | FIGS. 40, |
| | H289N_P291S_F14.VT8_Y712I.SOSL_GS.PC_{—} | production | 58 (SEQ |
| | gp150.755_CD5ss | as mRNA) | ID NOs: 89, |
| | | | 327) |
| HV1303323 | CH0848.d949.10.17_MD64V1_E169K_D230N_{—} | Nt(e.g. for | FIGS. 40, |
| | H289N_P291S_T316W_F14_Y712I_DS.SOSL_{—} | production | 58 (SEQ |
| | GS.PC_gp150.712_CD5ss | as mRNA) | ID NO: 90, |
| | | | 328) |
| HV1303324 | CH0848.d949.10.17_MD64V1_E169K_D230N_{—} | Nt(e.g. for | FIGS. 40, |
| | H289N_P291S_T316W_F14.VT8_Y712I_DS.SOSL_{—} | production | 58 (SEQ |
| | GS.PC_gp150.712_CD5ss | as mRNA) | ID NO: 91, |
| | | | 329) |
| HV1303325 | CH0848.d949.10.17_MD64V1_E169K_D230N_{—} | Nt(e.g. for | FIGS. 40, |
| | H289N_P291S_F14.VT8_Y712I.SOSL_GS.PC_{—} | production | 58 (SEQ |
| | gp150.712_CD5ss | as mRNA) | ID NO: 92, |
| | | | 330) |
| DW-19 | V1swap DS-SOSIP | Nt(e.g. for | FIG. 40 |
| | | production | (SEQ ID |
| | | as mRNA) | NO: 76) |
| DW-21 | V1swap DS-SOSIP-2P | Nt(e.g. for | FIG. 40 |
| | | production | (SEQ ID |
| | | as mRNA) | NO: 77) |
| DW-34 | V1swap E169K DS-SOSIP-2P | Nt(e.g. for | FIG. 40 |
| | | production | (SEQ ID |
| | | as mRNA) | NO: 78) |
| DW-84 | V1swap E169K F14 chim SOSIP | Nt(e.g. for | FIG. 40 |
| | | production | (SEQ ID |
| | | as mRNA) | NO: 79) |
| DW-97 | V1swap E169K F14 DSchim SOSIP | Nt(e.g. for | FIG. 40 |
| | | production | (SEQ ID |
| | | as mRNA) | NO: 80) |
| HV1303479 | CH0848.d949.10.17_V1swap_E169K_S148G_{—} | Nt(e.g. for | FIG. 58 |
| | Q328M_F14_DS_SOSIP_MD2_MD33_gp160_CD5ss | production | (SEQ ID |
| | | as mRNA) | No: 304) |
| HV1303480 | CH0848.d949.10.17_V1swap_E169K_S148G_{—} | Nt(e.g. for | FIG. 58 |
| | Q328M_F14_DS_SOSIP_MD2_MD33_Y712I_{—} | production | (SEQ ID |
| | gp160_CD5ss | as mRNA) | NO: 305) |
| HV1303481 | CH0848.d949.10.17_V1swap_E169K_S148G_{—} | Nt(e.g. for | FIG. 58 |
| | Q328M_F14_DS_SOSIP_MD2_MD33_Y712I.LL856AA_{—} | production | (SEQ ID |
| | gp160_CD5ss | as mRNA) | NO: 306) |
| HV1303482 | CH0848.d949.10.17_V1swap_E169K_S148G_{—} | Nt(e.g, for | FIG. 58 |
| | Q328M_F14_DS_SOSIP_MD2_MD33_Y712I_{—} | production | (SEQ ID |
| | gp150.755_CD5ss | as mRNA) | NO: 307) |
| HV1303483 | CH0848.d949.10.17_V1swap_E169K_S148G_{—} | Nt(e.g. for | FIG. 58 |
| | Q328M_F14_DS_SOSIP_MD2_MD33_Y712I_{—} | production | (SEQ ID |
| | gp150.712_CD5ss | as mRNA) | NO: 308) |
| HV1303484 | CH0848.d949.10.17_V1swap_E169K_S148G_{—} | Nt(e.g. for | FIG. 58 |
| | Q328M_F14_DS_SOSIP_MD2_MD33_Y712I_{—} | production | (SEQ ID |
| | gp150.TM1_CD5ss | as mRNA) | NO: 309) |
| HV1303645 | CH0848.d949.10.17_V1swap_E169K_S148G_{—} | Nt(e.g. for | FIG. 58 |
| | Q328M_D230N_H289N_P291S_S306L_R308L_{—} | production | (SEQ ID |
| | T316W_F14_DS_SOSIP_MD2_MD33_Y712I_{—} | as mRNA) | NO: 310) |
| | gp150.755_CD5ss | | |
| HV1303646 | CH0848.d949.10.17_V1swap_E169K_S148G_{—} | Nt(e.g. for | FIG. 58 |
| | Q328M_D230N_H289N_P291S_S306L_R308L_{—} | production | (SEQ ID |
| | T316W_F14_DS_SOSIP_MD2_MD33_Y712I_{—} | as mRNA) | NO: 311) |
| | gp150.712_CD5ss | | |
| HV1303647 | CH0848.d949.10.17_V1swap_E169K_S148G_{—} | Nt(e.g. for | FIG. 58 |
| | Q328M_D230N_H289N_P291S_S306L_R308L_{—} | production | (SEQ ID |
| | T316W_F14_DS_SOSIP_MD2_MD33_Y712I_{—} | as mRNA) | NO: 312) |
| | gp150.TM1_CD5ss | | |
| HV1303621 | CH0848.d949.10.17_V1swap_E169K_S148G_{—} | Nt(e.g. for | FIG. 58 |
| | Q328M_S306L_R308L_T316W_F14_DS_SOSIP_{—} | production | (SEQ ID |
| | MD2_MD33_Y7121.LL856AA_gp160_CD5ss | as mRNA) | NO: 313) |
| HV1303622 | CH0848.d949.10.17_V1swap_E169K_S148G_{—} | Nt(e.g. for | FIG. 58 |
| | Q328M_S306L_R308L_T316W_F14_DS_SOSIP_{—} | production | (SEQ ID |
| | MD2_MD33_Y712I_gp150.755_CD5ss | as mRNA) | NO: 314) |
| HV1303623 | CH0848.d949.10.17_V1swap_E169K_S148G_{—} | Nt(e.g. for | FIG. 58 |
| | Q328M_S306L_R308L_T316W_F14_DS_SOSIP_{—} | production | (SEQ ID |
| | MD2_MD33_Y712I_gp150.712_CD5ss | as mRNA) | NO: 315) |
| HV1303624 | CH0848.d949.10.17_V1swap_E169K_S148G_{—} | Nt(e.g. for | Fig, 58 |
| | Q328M_S306L_R308L_T316W_F14_DS_SOSIP_{—} | production | (SEQ ID |
| | MD2_MD33_Y712I_gp150.TM1_CD5ss | as mRNA) | NO: 316) |
| HV1303625 | CH0848.d949.10.17_V1swap_E169K_S148G_{—} | Nt(e.g. for | FIG. 58 |
| | Q328M_S306L_R308L_T316W_F14_DS_SOSIP_{—} | production | (SEQ ID |
| | MD2_MD33_25lnGSERK_VRCFerritin | as mRNA) | NO: 317) |
| HV1303626 | CH0848.d949.10.17_V1swap_E169K_S148G_{—} | Nt(e.g. for | FIG. 58 |
| | Q328M_S306L_R308L_T316W_F14_DS_SOSIP_{—} | production | (SEQ ID |
| | MD2_MD33_14lnGSERK_VRCFerritin | as mRNA) | NO: 318) |
| HV1303627 | CH0848.d949.10.17_V1swap_E169K_S148G_{—} | Nt(e.g. for | FIG. 58 |
| | Q328M_S306L_R308L_T316W_F14_DS_SOSIP_{—} | production | (SEQ ID |
| | MD2_MD33_10lnQQ_VRCFerritin | as mRNA) | NO: 319) |

Provided are immunogens derived from the HIV-1 envelope sequences from the CH848 infected individual. Several strategies have been used to develop these immunogens and encoding nucleic acids.

Modifications of nucleic acids encoding the inventive envelopes may include:
S′UTR including
aGcATAAAAGTCTCAACACAACATATACAAAACAAACGAATCTCAAGCAA TCAAGCATTCTACTTCTATTGCAGCAATTTAAATCATTTCTTTTAAAGCAA AAGCAATTTTCTGAAAATTTTCACCATTTACGAACGATAGCGCT (SEQ ID NO: 16). Without being bound by theory, this modification is an improved 5′ UTR sequence for mRNA stability and half-life from screens. See Messenger RNA-Based Vaccines Against Infectious Diseases. Alameh M G, Weissman D, Pardi N.Curr Top Microbiol Immunol. 2020 Apr. 17. Doi: 10.1007/82_2020_202. PMID: 32300916.
3′UTR including
actagtAGTGACTGACTAGGATCTGGTTACCACTAAACCAGCCTCAAGAACAC CCGAATGGAGTCTCTAAGCTACATAATACCAACTTACACTTACAAAATGTT GTCCCCCAAAATGTAGCCATTCGTATCTGCTCCTAATAAAAAGAAAGTTTC TTCACATTCT (SEQ ID NO: 17). Without being bound by theory, this modification is an improved 5′ UTR sequence for mRNA stability and half-life from screens. See Messenger RNA-Based Vaccines Against Infectious Diseases. Alameh M G, Weissman D, Pardi N.Curr Top Microbiol Immunol. 2020 Apr. 17. Doi: 10.1007/82_2020_202. PMID: 32300916.
poly A (immediately after 3′UTR) includes
AAAAAAAAAAAAAAAAAAAAAAAAAAAAAAAAAAAAAAAAAAAAAAA AAAAAAAAAAAAAAAAAAAAAAAAAAAAAAAAAAAAAAAAAAAAAAA AAAAAAA (SEQ ID NO: 10). Without being bound by theory, this modification is an improved poly A tail sequence for mRNA stability and half-life. See Jalkanen et al. Semin Cell Dev Biol. 34:24-32 (2014).
mRNA codon optimization includes a reverse translation of protein amino acid sequence to optimal codons. Without being bound by theory, this modification codon optimization is performed as follow: amino acid sequence is reverse translated into an DNA sequence using a modified mammalian codon usage table. The table increases both the CIA and the GC content of the mRNA. The reverse translated sequence (or mRNA sequence) is modeled into mFold and Delta H/Delta G computed, and the sequence with the lowest free energy is selected. In some cases, the codons can be replaced in specific locations to relax the tridimentional structure of the optimized mRNA. The sequence is then cloned between the S′UTR and 3′UTR above. See Leppek et al. Nature Communications 13:1536 (2022).

The exemplary constructs provided herein, see e.g., FIG. 40 or 58, include various combinations of these modifications. Any modification or combination of the modifications described herein, including but not limited, to different versions of soluble proteins, different versions of membrane expressed proteins, stabilization mutations, furin cleavage site mutations, signal peptides, and/or cytoplasmic tail modifications can be applied to any HIV-1 envelope protein sequence described herein.

### EXEMPLARY EMBODIMENTS

In certain aspects, the invention provides a recombinant HIV-1 envelope comprising all the consecutive amino acids after the signal peptide of SEQ ID NOs: 19-30, 33, 35-45, 52-63 or 288-303. In some embodiments, the recombinant HIV-1 envelope comprises all the consecutive amino acids after the signal peptide of SEQ ID NO: 19, 23, 290 or 291.

In certain aspects, the invention provides a composition comprising any one of the HIV-1 envelopes described herein and a carrier. In some embodiments, the composition is an immunogenic composition. In some embodiments, the HIV-1 envelope is a protomer comprised in a trimer. In some embodiments, the HIV-1 envelope is comprised in a stable trimer.

In certain aspects, the invention provides a composition comprising a nanoparticle and a carrier, wherein the nanoparticle comprises any one of the HIV-1 envelopes described herein. In some embodiments, the nanoparticle is a ferritin self-assembling nanoparticle. In some embodiments, the composition is an immunogenic composition.

In certain aspects, the invention provides a composition comprising a nanoparticle and a carrier, wherein the nanoparticle comprises any one of the trimers described herein. In some embodiments, the nanoparticle is a ferritin self-assembling nanoparticle. In some embodiments, the nanoparticle comprises multimers of trimers. In some embodiments, the nanoparticle comprises 1-8 trimers. In some embodiments, the composition is an immunogenic composition.

In certain aspects, the invention provides a method of inducing an immune response in a subject comprising administering an immunogenic composition comprising any one of the recombinant HIV-1 envelopes described herein or compositions described herein in an amount sufficient to induce an immune response. In some embodiments, the immunogenic composition is administered as a prime. In some embodiments, the immunogenic composition is administered as a boost.

In certain aspects, the invention provides a nucleic acid encoding any of the HIV-1 envelopes described herein. In certain aspects, the invention provides a nucleic acid sequence comprising SEQ ID NOs 64-94 or 304-330. In some embodiments, the nucleic acid is a mRNA. In some embodiments, the mRNA is encapsulated in a lipid nanoparticle.

In certain aspects, the invention provides a composition comprising the nucleic acid described herein and a carrier. In some embodiments, the composition is an immunogenic composition. In some embodiments, the composition further comprises an adjuvant. In some embodiments, the nucleic acid is operably linked to a promoter, and optionally the nucleic acid is inserted in an expression vector.

In certain aspects, the invention provides a composition comprising a nanoparticle and a carrier, wherein the nanoparticle comprises any one of the nucleic acids described herein. In some embodiments, the nucleic acid is a mRNA. In some embodiments, the nanoparticle is a lipid nanoparticle.

In certain aspects, the invention provides a method of inducing an immune response in a subject comprising administering an immunogenic composition comprising the nucleic acid described herein or the composition described herein in an amount sufficient to induce an immune response. In some embodiments, the method further comprises administering an agent which modulates host immune tolerance. In some embodiments, the nucleic acid administered is a mRNA. In some embodiments, the nucleic acid is encapsulated in a lipid nanoparticle. In some embodiments, the method further comprises administering one or more additional HIV-1 immunogens to induce a T cell response. In some embodiments, the immunogenic composition is administered as a prime. In some embodiments, the immunogenic composition is administered as a boost.

In certain aspects, the invention provides a method of inducing an immune response comprising administering an immunogenic composition comprising a prime immunogen comprising all the consecutive amino acids after the signal peptide of SEQ ID NOs: 19-30, 33, 35-45, 52-63 or 288-303 or a nucleic acid sequence encoding all the consecutive amino acids after the signal peptide of SEQ ID NOs: 19-30, 33, 35-45, 52-63 or 288-303 followed by at least one boost immunogen from Table 3 or Table 4, wherein the boost immunogens are administered in the order appearing in Table 4, in an amount sufficient to induce an immune response. In some embodiments, the prime or boost immunogen are administered as a nanoparticle. In some embodiments, the nanoparticle is a ferritin self-assembling nanoparticle. In some embodiments, the prime or boost immunogen are administered as a nucleic acid mRNA-LNP formulation.

In certain aspects, the invention provides an immunogenic composition or composition described herein, wherein the composition comprises at least two different recombinant HIV-1 envelopes or nucleic acids encoding a HIV-1 envelope, or a combination thereof.

In certain aspects, the invention provides an immunogenic composition comprising a first immunogen and a second immunogen, wherein the first immunogen is a HIV-1 envelope comprising all the consecutive amino acids after the signal peptide of SEQ ID NOs: 19-30, 33, 35-45, 52-63 or 288-303, or a nucleic acid sequence comprising SEQ ID NOs: 64-94 or 304-330, and wherein the second immunogen is a different HIV-1 envelope comprising all the consecutive amino acids after the signal peptide of SEQ ID NOs: 19-30, 33, 35-45, 52-63 or 288-303, or a nucleic acid sequence comprising SEQ ID NOs: 64-94 or 304-330. In some embodiments, at least one of the first immunogen and the second immunogen is a recombinant HIV-1 envelope protein sequence. In some embodiments, the first immunogen and the second immunogen are both a recombinant HIV-1 envelope protein sequence. In some embodiments, at least one of the first immunogen and the second immunogen is a nucleic acid. In some embodiments, the first immunogen and the second immunogen are both a nucleic acid. In some embodiments, the nucleic acid is an mRNA. In some embodiments, the mRNA is encapsulated in an LNP. In some embodiments, the immunogenic composition further comprises one or more additional immunogens, wherein the one or more additional immunogens is different to the first and second immunogens. In some embodiments, the immunogenic composition comprises a carrier. In some embodiments, the immunogenic composition further comprises an adjuvant.

In certain aspects, the invention provides a method of inducing an immune response in a subject comprising administering the immunogenic composition as described herein in an amount sufficient to induce an immune response. In some embodiments, the method further comprises administering an agent which modulates host immune tolerance.

In certain aspects, the invention provides a recombinant trimer comprising three identical protomers of an envelope comprising all the consecutive amino acids after the signal peptide of SEQ ID NOs: 19-30, 33, 35-45, 52-63 or 288-303. In some embodiments, the envelope comprises all the consecutive amino acids after the signal peptide of SEQ ID NO: 19. In some embodiments, the envelope comprises all the consecutive amino acids after the signal peptide of SEQ ID NO: 23. In some embodiments, the envelope comprises all the consecutive amino acids after the signal peptide of SEQ ID NO: 290. In some embodiments, the envelope comprises all the consecutive amino acids after the signal peptide of SEQ ID NO: 291.

In certain aspects, the invention provides an immunogenic composition comprising the recombinant trimer described herein and a carrier. In certain aspects, the invention provides a composition comprising a nanoparticle and a carrier, wherein the nanoparticle comprises any one of the trimers described herein. In some embodiments, the nanoparticle is ferritin self-assembling nanoparticle. In some embodiments, the nanoparticle comprises multimers of trimers. In some embodiments, the nanoparticle comprises 1-8 trimers. The invention is further described in the following non-limiting examples.

### EXAMPLES

### Example 1—Engineering Broadly Reactive Env Immunogens To Stimulate Multiple V3 Glycan bnAb Precursors

One of the major impediments to the elicitation of an effective immune response upon HIV vaccination is the low frequency of V3 glycan bnAb precursors which are present in the human repertoire. In an effort to specifically engage and stimulate the proliferation of these rare B cells, HIV Envelopes (Envs) have been engineered which are capable of binding to unmutated B cell receptors that have the potential to mature into V3 glycan bnAbs. For example, CH848 10.17DT is a clade C Env that has been modified to bind to the UCA from the DH270 lineage with high affinity. Similarly, the N332-GT5 Env binds the UCA from the BG18 lineage. These engineered Envs represent significant advances towards being able to stimulate a V3 glycan bnAb response, but unfortunately they are both relatively specific for the B cell lineages that they were designed to target. In other words, CH848 10.17DT is incapable of binding to the BG18 UCA and N332-GT5 is incapable of binding to the DH270 UCA. Therefore, the favorable characteristics from both of these Envs were combined into a single immunogen in an effort to expand the number of V3 glycan UCAs which can be stimulated during vaccination.

FIG. 1 depicts the different modes of binding that the DH270 UCA3 and the BG18 UCA use to bind to their respective targets. A gp120 monomer of CH848 10.17DT in the prefusion conformation is shown in teal ribbons, bound by DH270 UCA3 Fab, shown as a transparent molecular surface with the heavy chain colored blue and the light chain colored gray (FIG. 1, top left). A zoomed-in view of the DH270 UCA3 binding interface is displayed to illustrate that the angle of binding is such that no significant contacts are formed with the neighboring V1 loop. Critical contact residues and glycans are shown as sticks, with oxygen atoms colored red and nitrogen atoms colored blue. This mode of binding can be contrasted with N332-GT5, which is shown in the prefusion conformation as orange ribbons, bound by BG18 UCA Fab, shown as a transparent molecular surface with the heavy chain colored purple and the light chain colored white (FIG. 1, top right). A zoomed-in view of the BG18 UCA binding interface is displayed, and critical contacting residues shown as sticks, with oxygen atoms colored red, nitrogen atoms colored blue and sulfur atoms colored yellow. The BG18 UCA straddles the V1 loop, forming critical contacts with the basic residues at the tip of the N332-GT5 V1 loop. In an effort to generate a single Env that could bind to both UCAs, residues 132-154 (HXB2 numbering) of the N332-GT5 V1 loop were transfected into the CH848 10.17DT Env, thus generating the V1swap trimer (FIG. 1, bottom).

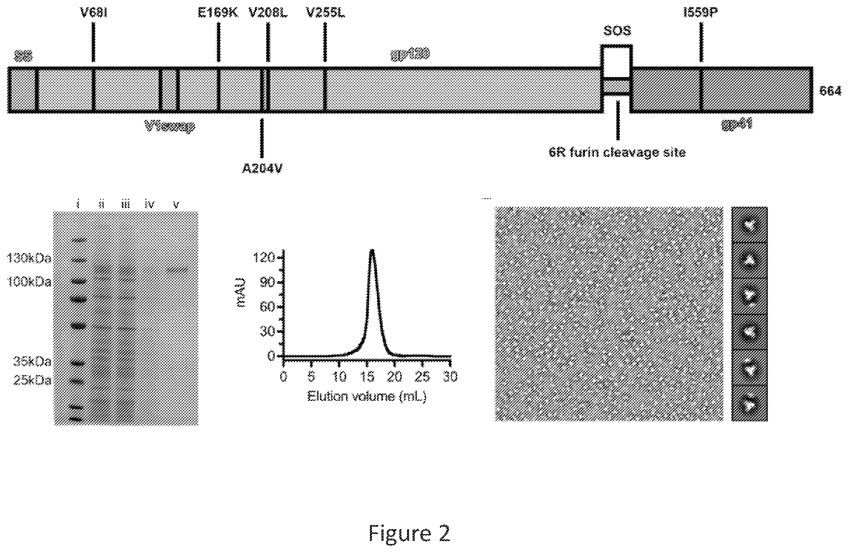

FIG. 2 shows the subsequent expression and purification of this V1swap Env. A primary sequence diagram is shown, depicting the soluble V1swap Env construct. The CH848 10.17 gp 120 is colored teal, the V1swap mutations are colored orange and the chimeric BG505 gp41 is colored pink. Portions of the construct which are post-translationally modified and are not present in the mature Env are colored gray. The various mutations that were made throughout the Env are labeled. “SS”=signal sequence, “SOS”=A501C+T605C, “664” signifies that the construct terminates after residue 664 (FIG. 2, top). An SDS-PAGE gel showing the purification of V1swap Env by PGT145 affinity chromatography is shown (FIG. 2, bottom left). Lane i shows a molecular weight ladder, with relevant standards labeled. Lane ii shows filtered supernatant after transient transfection. Lane iii shows the flowthrough from a PGT145 column. Lane iv shows the wash and lane v corresponds to the elution. The size exclusion chromatogram of V1swap after PGT145 purification is shown, generated using a Superose 6 Increase 10/300 GL column (FIG. 2, bottom middle). A negative stain electron micrograph of purified V1swap is shown, along with corresponding 2D class averages showing prefusion trimers (FIG. 2, bottom right) . . . .

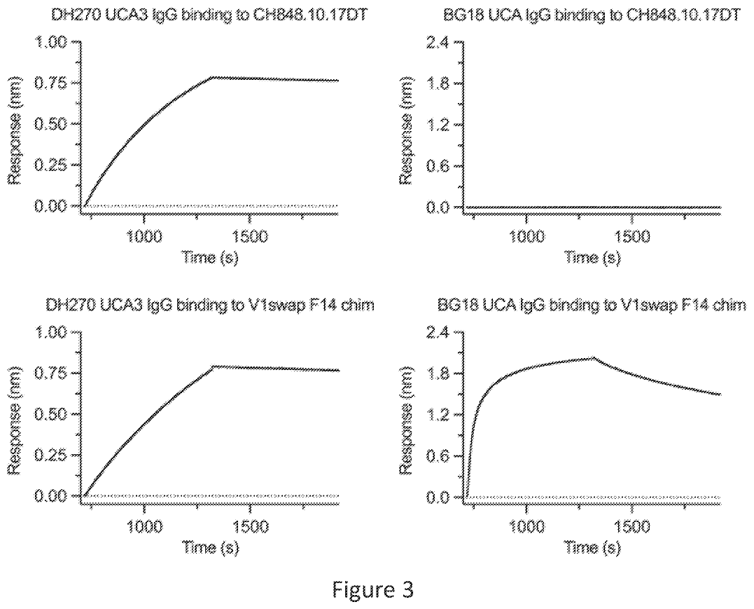

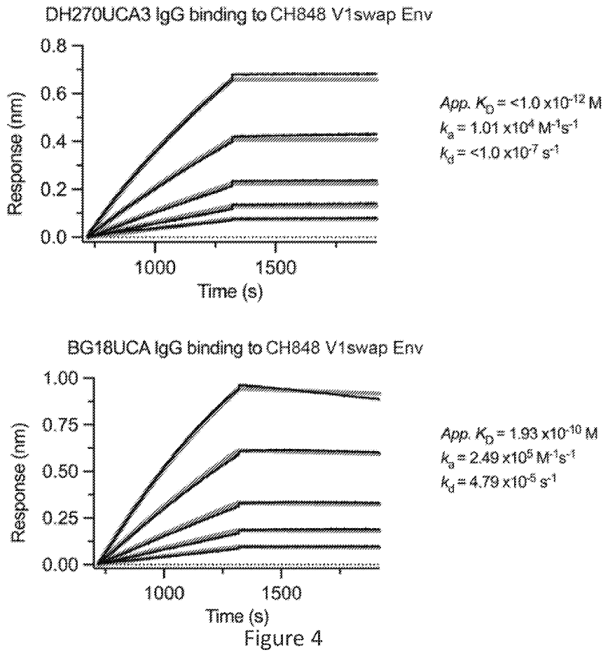

Biolayer interferometry (BLI) was then performed to measure whether the V1swap Env exhibited expanded UCA reactivity relative to CH848 10.17DT (FIG. 3). Biolayer interferometry (BLI) sensorgrams are shown for the interactions between CH848 10.17DT Env and DH270 UCA3 IgG (FIG. 3, top left) and BG18 UCA IgG (FIG. 3, top right). As expected, CH848 10.17DT bound to the DH270 UCA3 with high affinity, but no binding could be detected between CH848 10.17DT and the BG18 UCA. BLI sensorgrams are also shown for the interactions between V1swap Env and DH270 UCA3 IgG (FIG. 3, bottom left) and BG18 UCA IgG (FIG. 3, bottom right). V1swap retains high-affinity binding to the DH270 UCA3, but we can also now observe a robust binding interaction to the BG18 UCA, indicating that transferring in the V1 loop from N332-GT5 was successful in expanding the UCA reactivity. Additional BLI experiments were then performed to more precisely measure the binding interactions between V1swap Env and both the DH270 UCA3 (FIG. 4, top and the BG18 UCA (FIG. 4, bottom). Both UCAs bound with subnanomolar apparent affinities.

The V1swap Env as an mRNA-encoded gp160 was produced. This construct was then transfected into 293F cells, which were then tested for binding against a panel of neutralizing and non-neutralizing Env-directed antibodies by flow cytometry (FIG. 5). The influenza HA-directed CH65 antibody was also included as a negative control. Despite robust binding to both the DH270 UCA3 and the BG18 UCA, along with many of the neutralizing antibodies that were tested, binding of antibodies which are targeted against non-neutralizing epitopes, such as F105, A32 and 19b, was observed, suggesting that the current iteration of the V1swap Env would benefit from additional stabilization to minimize exposure of these unproductive epitopes. Efforts to improve upon this initial construct are underway, in addition to testing the efficacy of several previously described stabilizing mutations using mammalian cell display to identify mutations that enhance the affinity of this current iteration of the V1swap trimer for the DH270 and BG18 UCAs.

Based on these preliminary data, the next generation of the V1swap immunogen was designed. The previously described stabilization strategies were tested to minimize non-neutralizing epitope exposure: DS, VT8, T316W, etc. Mammalian cell display was used to obtain mutants that enhance affinity for the DH270 and BG18 UCAs.

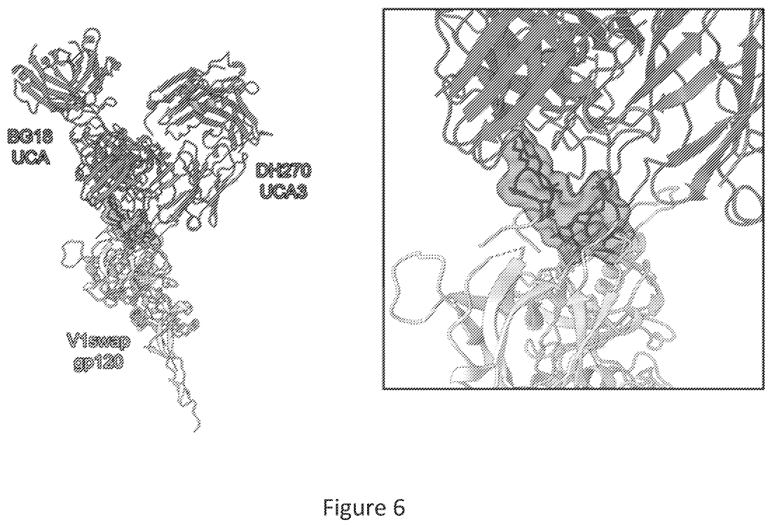

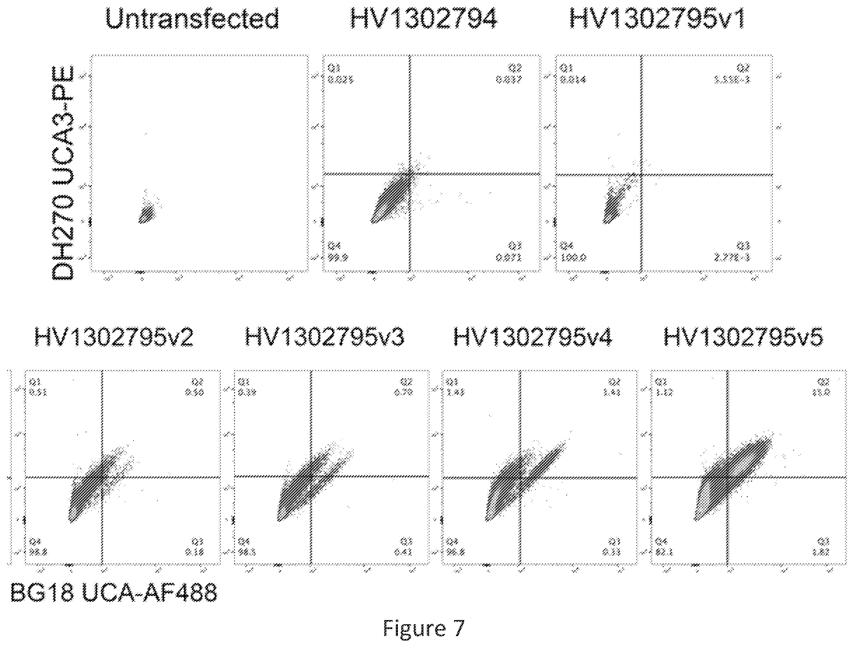

293T cells were transfected with a library of DNA encoding for a transmembrane-bound version of the V1swap Env with mutations to every possible amino acid at positions 135-141, 325-328, 330 and 417 (HXB2 numbering) (FIG. 6). A single gp 120 from the V1swap is shown in gray, with the BG18 UCA in red and the DH270 UCA3 in blue. Residues which have been mutated in the cell display library are shown in purple (FIG. 6). Cells expressing these mutant V1swap Envs were then stained with fluorescently tagged DH270 UCA3 and BG18 UCA and sorted by flow cytometry to isolate mutants that exhibited higher binding to both UCAs (FIG. 7). The y-axis of these flow plots shows binding to DH270 UCA3 conjugated to PE and the x-axis shows binding to BG18 UCA, conjugated to AlexaFluor488. Untransfected 293T cells have been included as a negative control and gates were set based on the unmutated V1swap prototype (HV1302794). HV1302795v1 is the unsorted library, and cells that bound to both the DH270 UCA3 and the BG18 UCA were sorted and enriched to generate HV1302795v2. This process was repeated for a total of four sorts, resulting in HV1302795v5, which now shows dramatic enrichment of cells that bind to DH270 UCA3 and BG18 UCA better than the prototype construct (HV1302794).

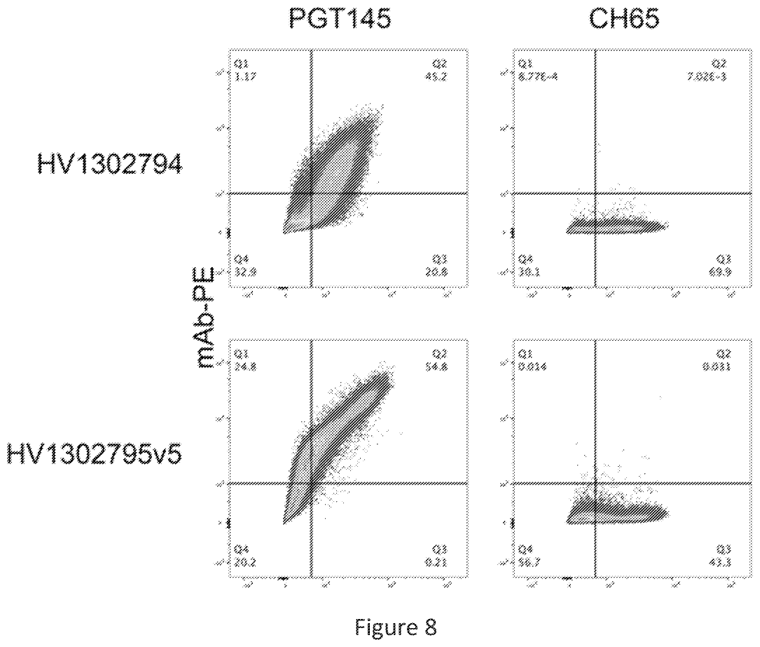

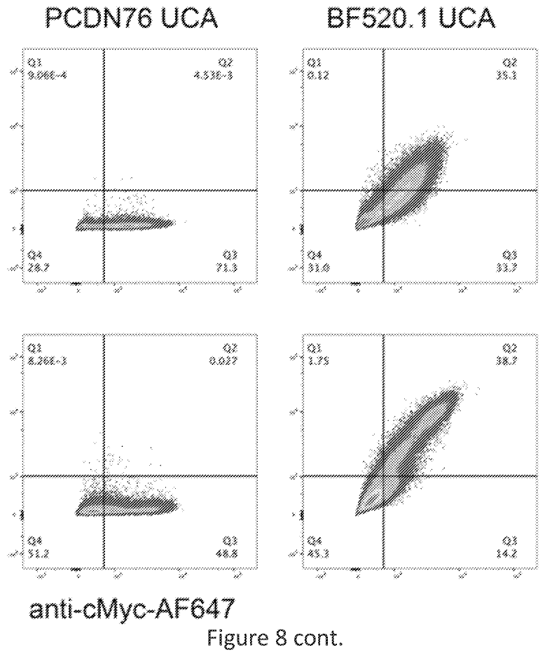

HV1302795v5 cells were then tested for binding to a small panel of monoclonal antibodies and the prototype V1swap Env (HV1302794) was also included as a control (FIG. 8). The y-axis shows binding of the monoclonal antibodies that are listed above and the x-axis shows binding to a c-Myc tag near the C-terminus of the membrane-immobilized Env, acting as a marker for the level of Env expression. In addition to enhanced binding to DH270 UCA3 and BG18 UCA, the HV1302795v5 cells also exhibit enhanced binding to the V2-apex antibody PGT145, suggesting that mutations that favor the folding and stabilization of the V1swap trimer overall may be selected. Furthermore, binding of BF520.1 UCA, a UCA from another V3 glycan bnAb lineage, to both the V1swap Env prototype and to the enriched HV1302795v5 cells was detected. CH65 was once again included as a negative control.

In summary, the existing V1swap immunogen binds to the UCAs from three V3 glycan bnAb lineages: DH270 UCA3, BG18 UCA and BF520.1 UCA. mRNA-encoded V1swap gp160 presented non-neutralizing epitopes, suggesting that additional stabilization is required. The next generation of the V1 swap trimer will incorporate additional, previously described stabilizing mutations and newly determined mutation from the mammalian cell display studies.

### Example 2—Engineering Single Env Immunogens To Stimulate V3 Glycan Precursors From Multiple Lineages

V3 glycan broadly neutralizing antibodies (bnAbs) are heterogenous in their immunogenetic and Env binding modes. This aspect necessitates immunogens that can interact with distinct precursors of V3-glycan bnAbs. HIV Envelopes have been engineered to specifically engage rare V3 glycan bnAb precursors and stimulate proliferation: CH848.10.17DT engages the DH270 UCA (Saunders et al., 2019) and N332-GT5 engages the BG18 UCA (Steichen et al., 2019). However, to date, the engineered envelopes have been shown to bind to the type of bnAb lineage used to design the envelope. Immunogens that can bind to different types of V3-glycan antibody lineages are still needed.

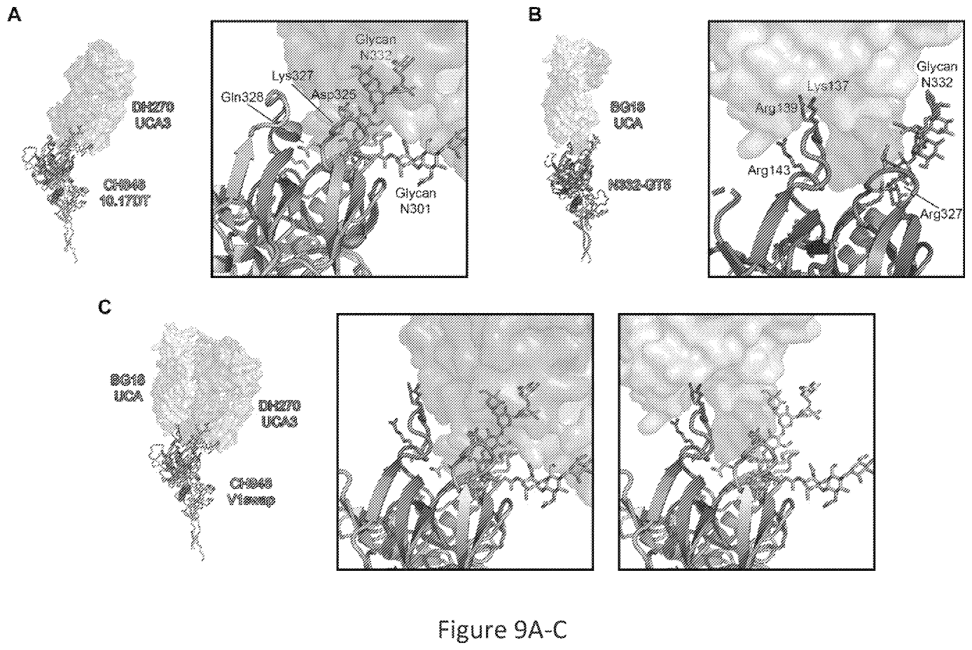

FIGS. 9A-C show the design rationale for CH848 V1swap immunogen.

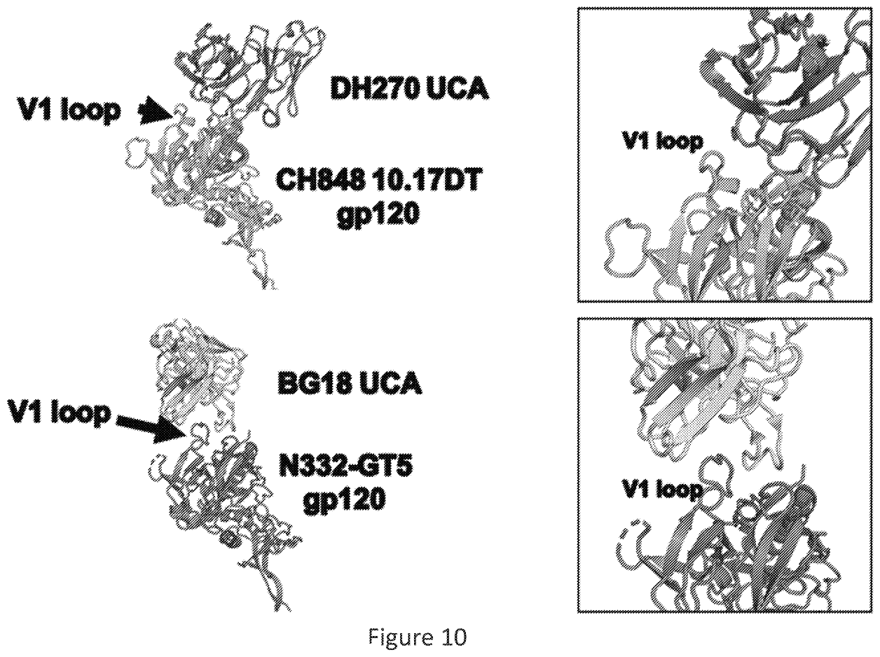

FIG. 10 shows that V3 glycan UCAs can be categorized based on their modes of Env engagement.

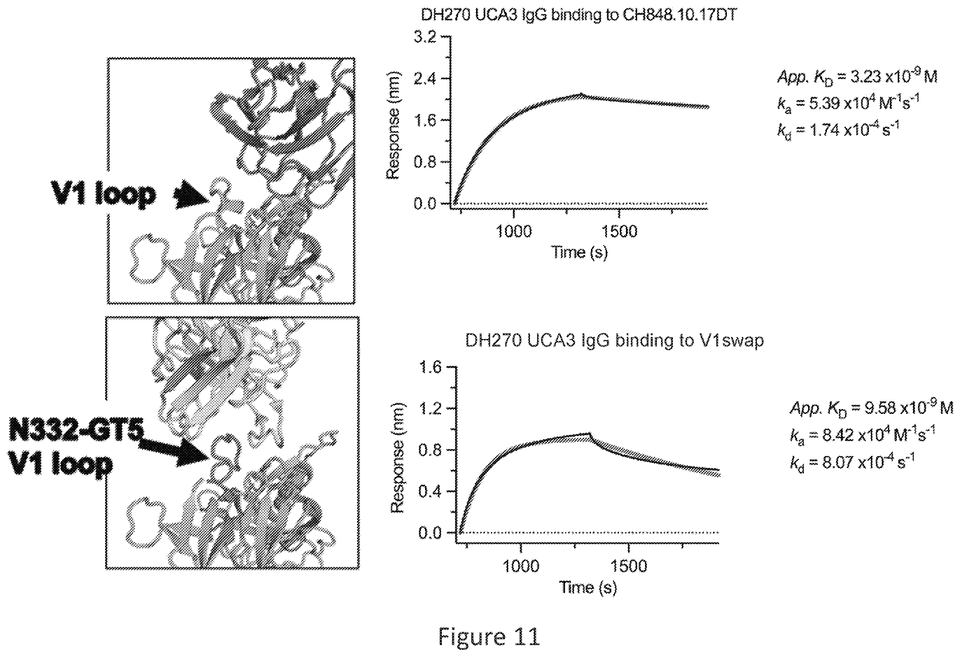

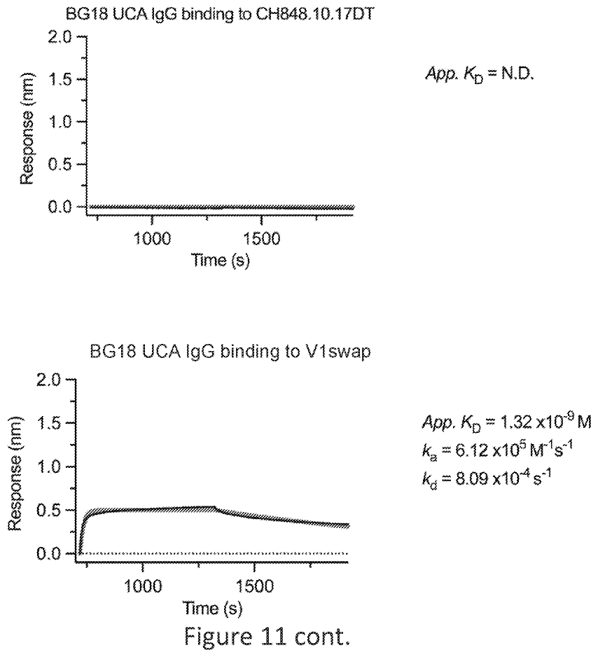

FIG. 11 shows the second generation V3-glycan germline targeting Env. It also shows that V1 transplantation from N332-GT5 into the CH848 10.17DT background permits BG18 UCA reactivity.

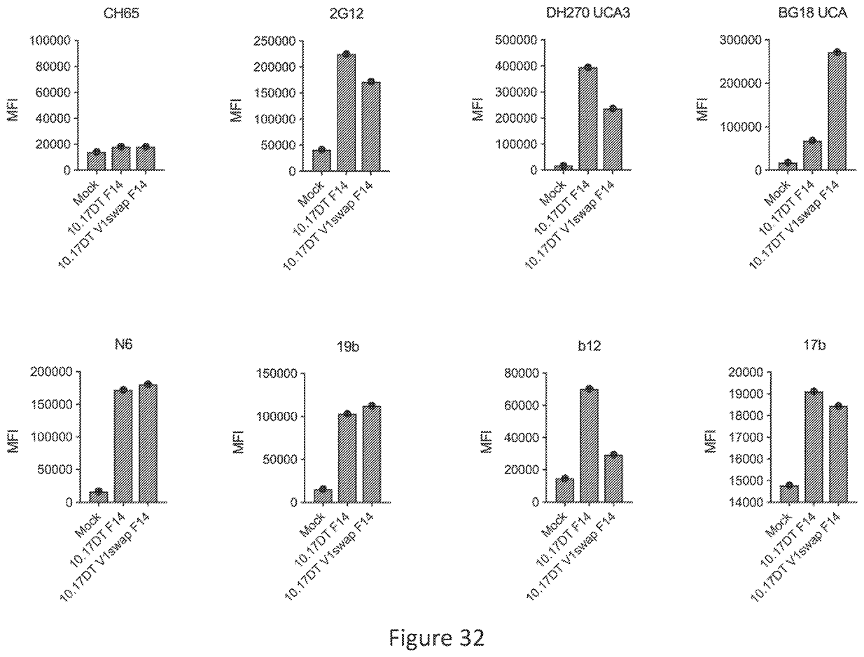

Previous study shows that mRNA-LNP encoding transmembrane stabilized gp160s were equally as immunogenic for expanding bnAb B cell precursors as were mRNA-LNP encoding ferritin-Env nanoparticles. See FIG. 32.

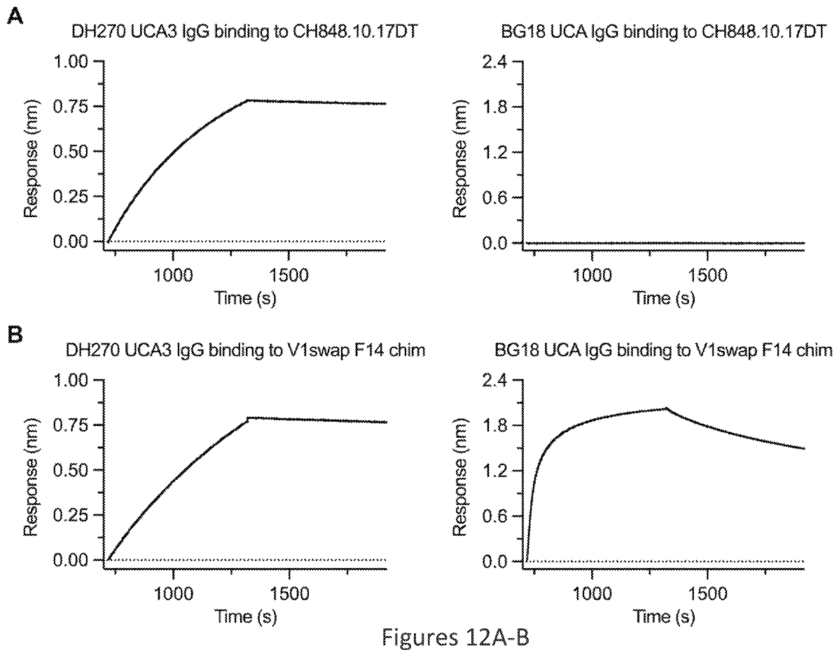

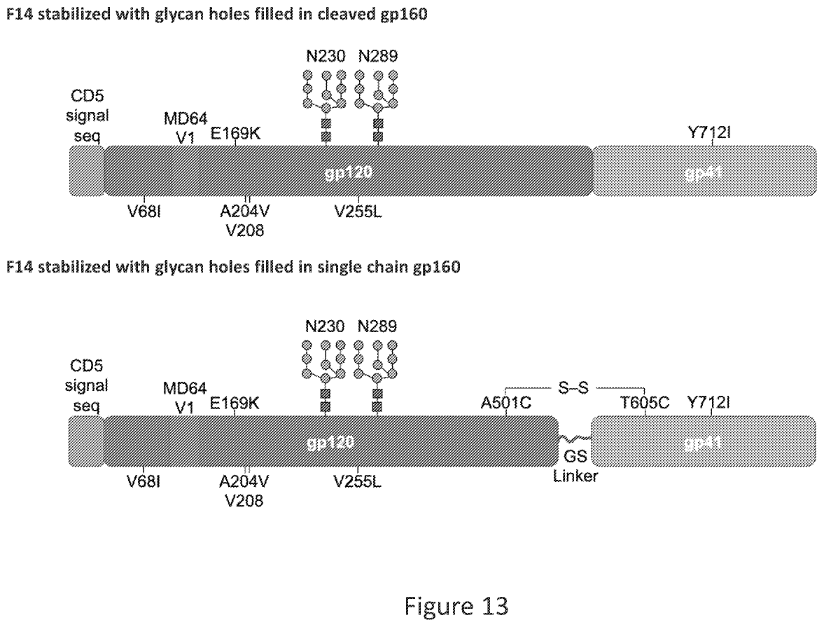

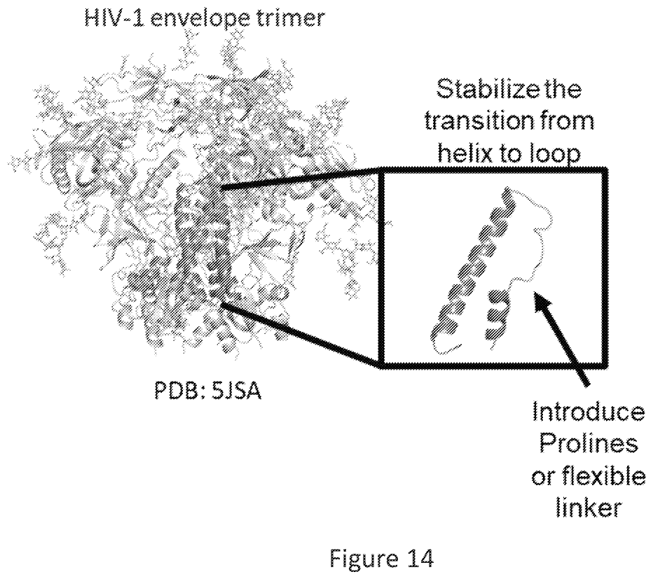

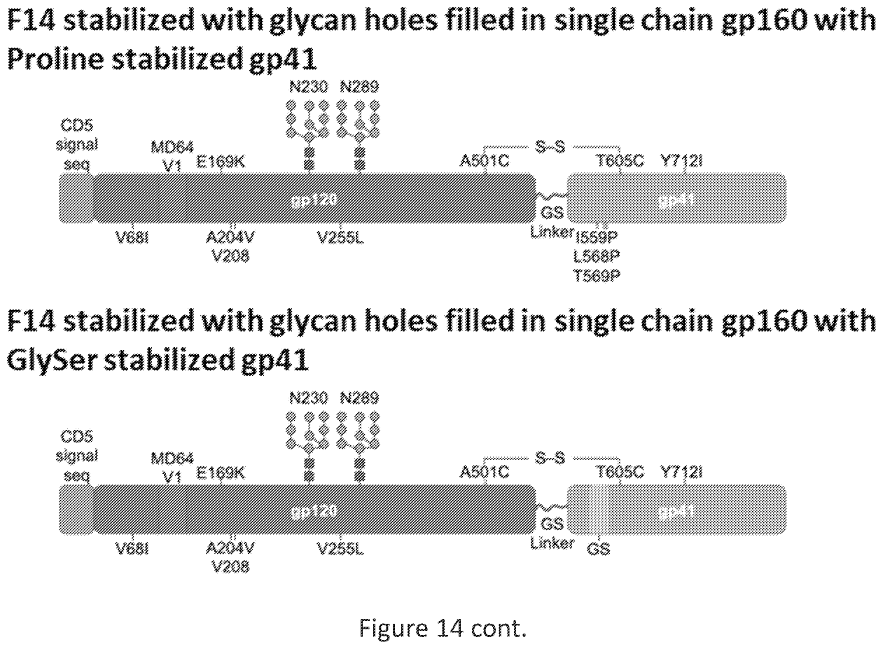

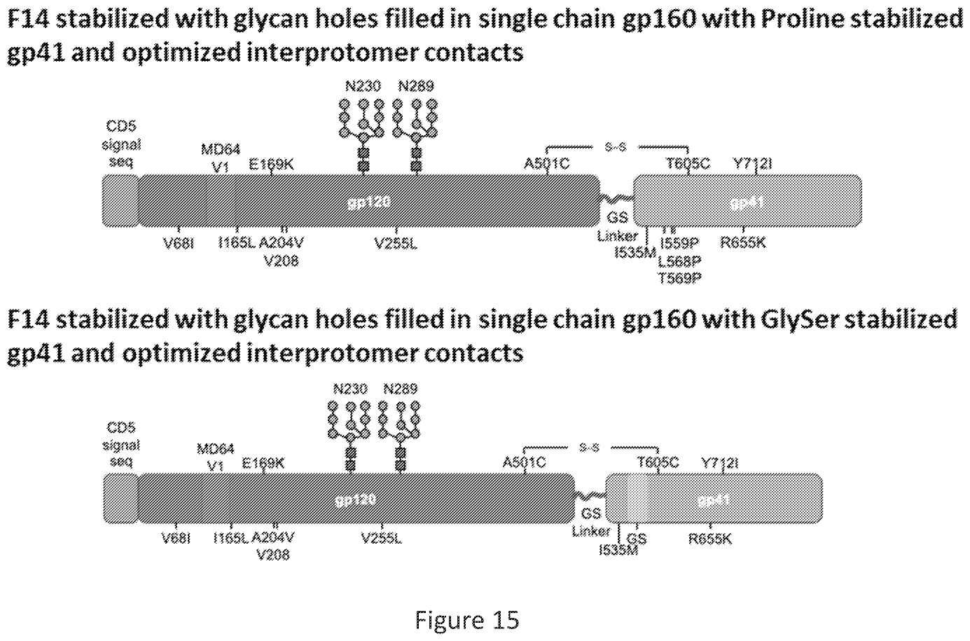

FIGS. 12A-B show that the V1swap mutations expand the UCA reactivity of the CH848 Env. FIG. 13 shows the second generation V3-glycan germline targeting Envs. It shows translation to mRNA expressed gp160s: F14 stabilized with glycan holes filled in cleaved gp160 and F14 stabilized with glycan holes filled in single chain gp160. FIG. 14 shows the optimization of gp41 stability with proline mutations or GlySer linkers to boost expression. Prolines or a flexible linker was introduced to stabilize the transition from helix to loop. FIG. 15 shows the optimization of gp41 stability by strengthening interprotomer interactions: F14 stabilized with glycan holes filled in single chain gp 160 with Proline stabilized gp41 and optimized interprotomer contacts, and F14 stabilized with glycan holes filled in single chain gp160 with GlySer stabilized gp41 and optimized interprotomer contacts.

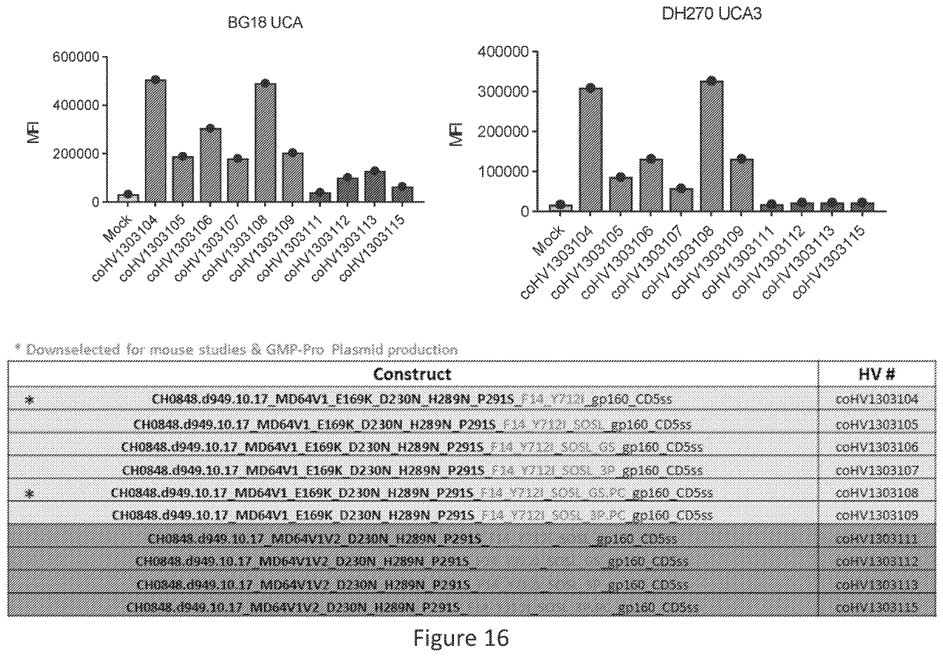

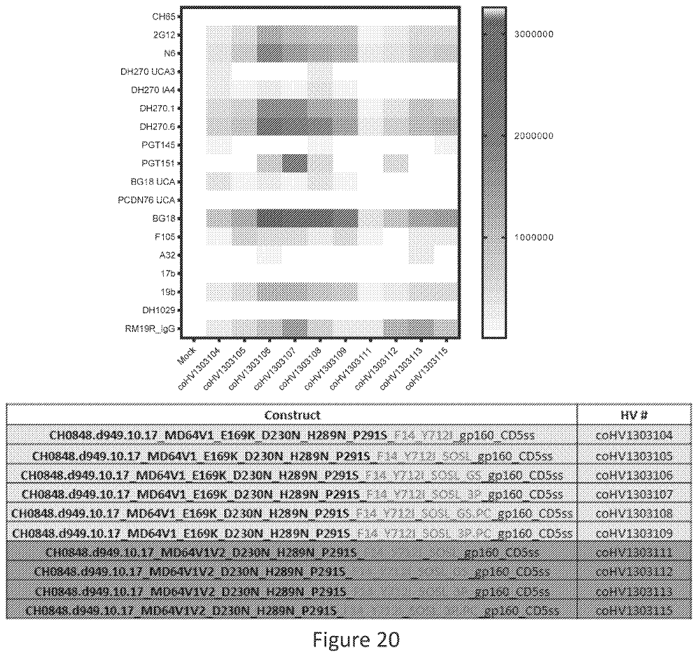

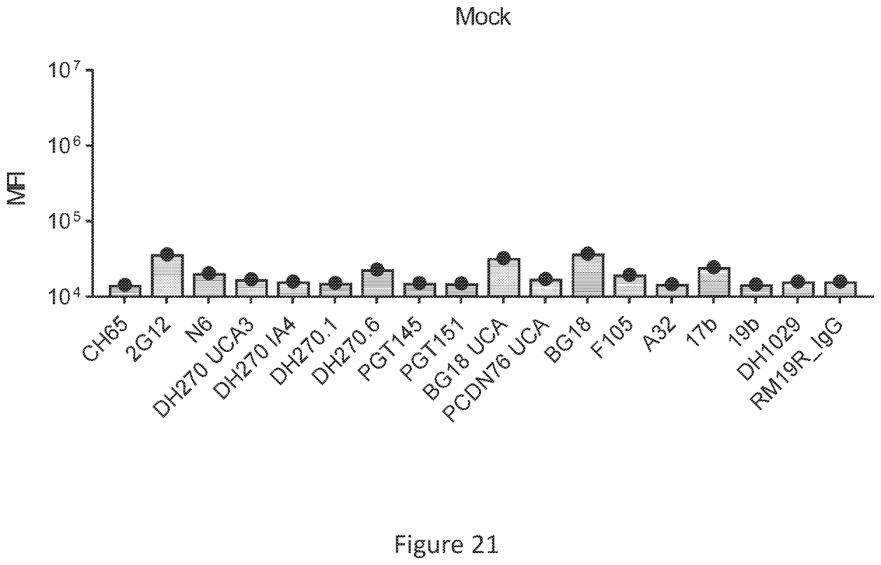

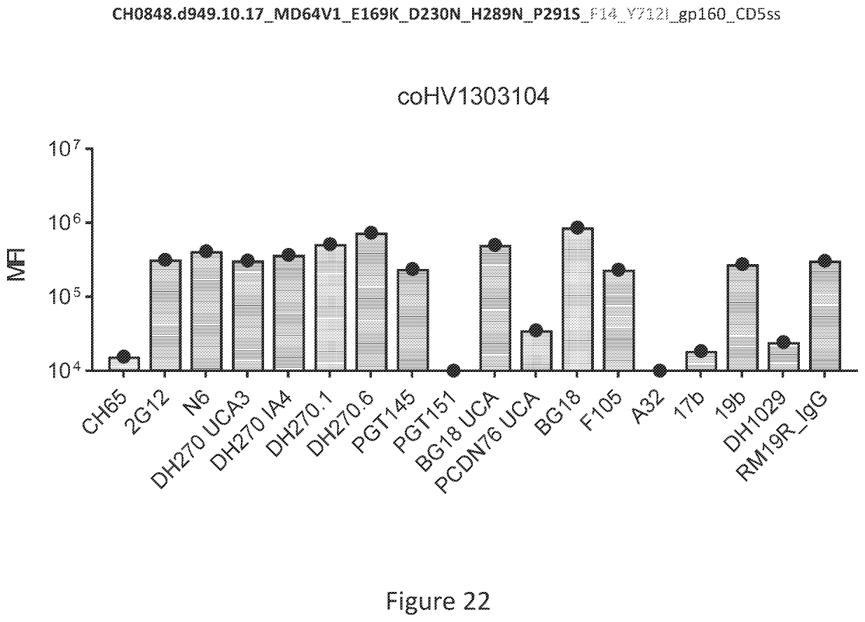

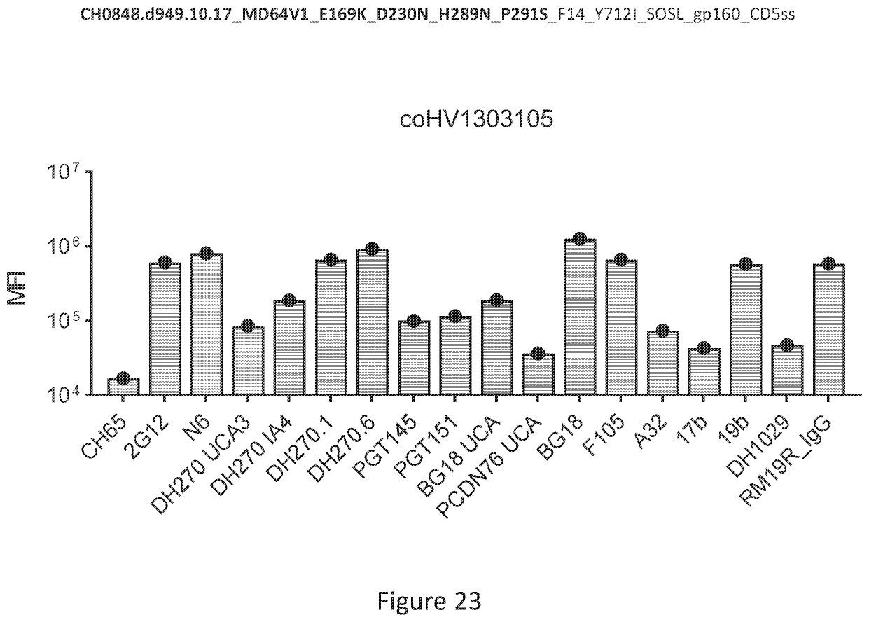

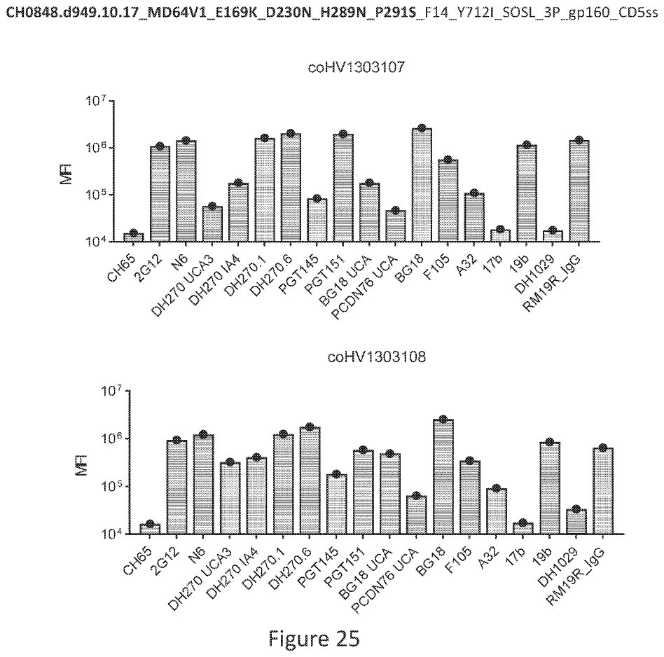

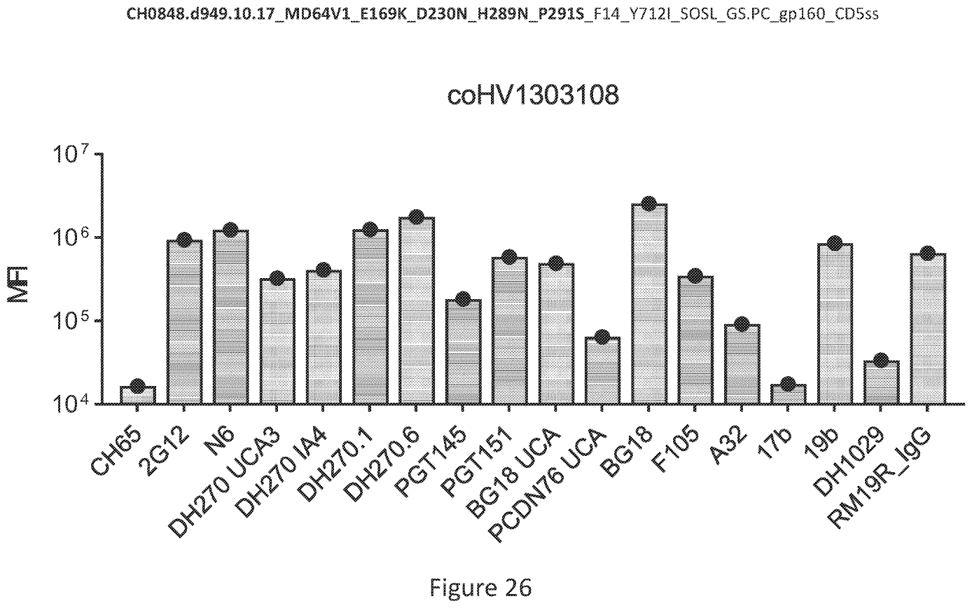

FIG. 16 shows the evaluation of V1swap gp160 mRNA variants by high-throughput flow cytometry. coHV1303104-coHV1303109, coHV1303111-HV1303113 and HV1303115 were tested. FIG. 20 shows the heatmap of all constructs with all antibodies. FIGS. 21-31 show the binding reactions of all constructs. FIG. 32 shows that CH848 V1swap envelope gp160 reacted with V3-glycan UCAs.

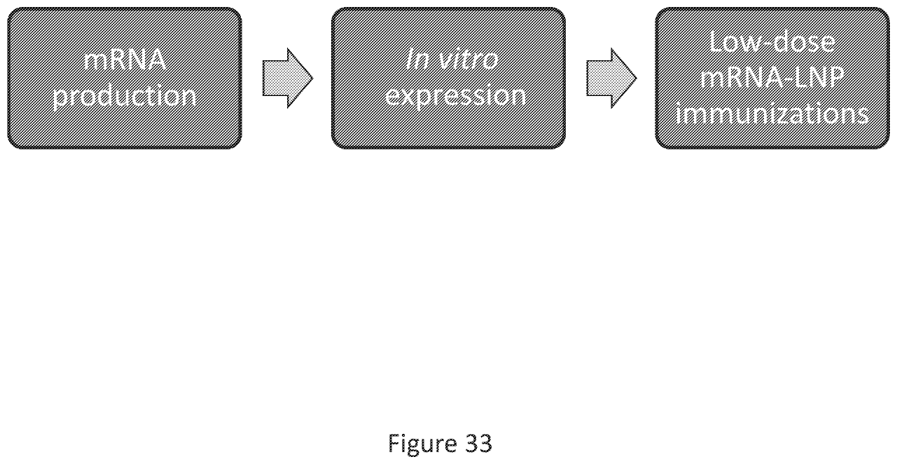

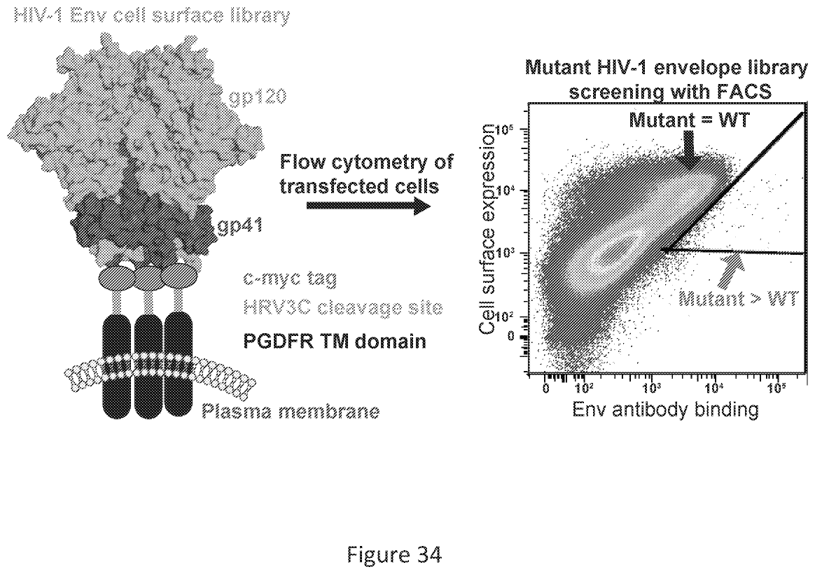

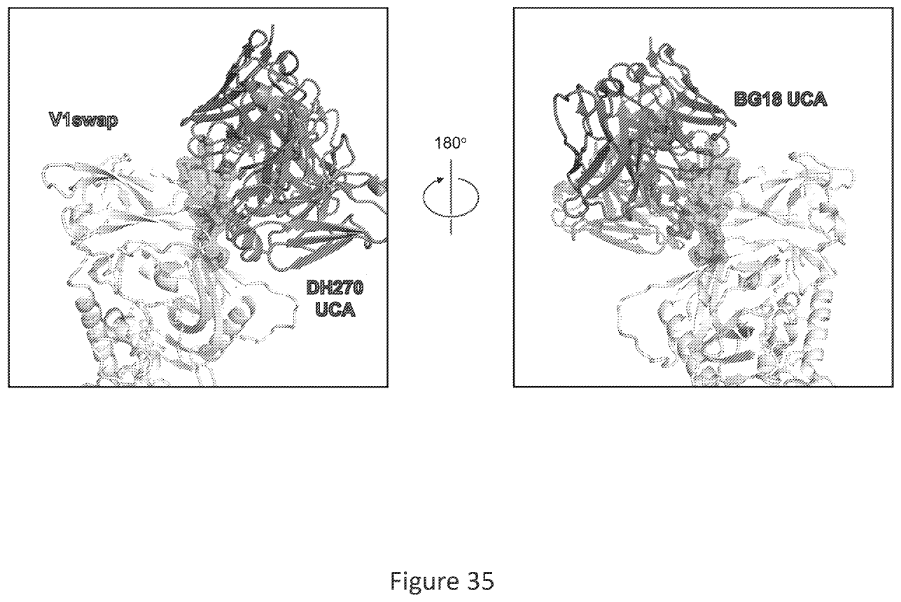

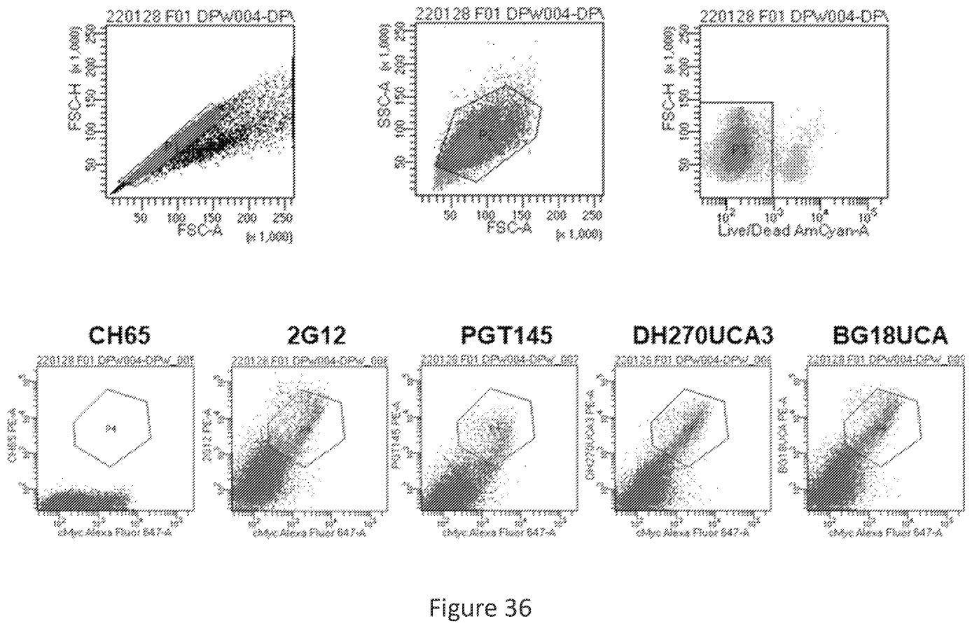

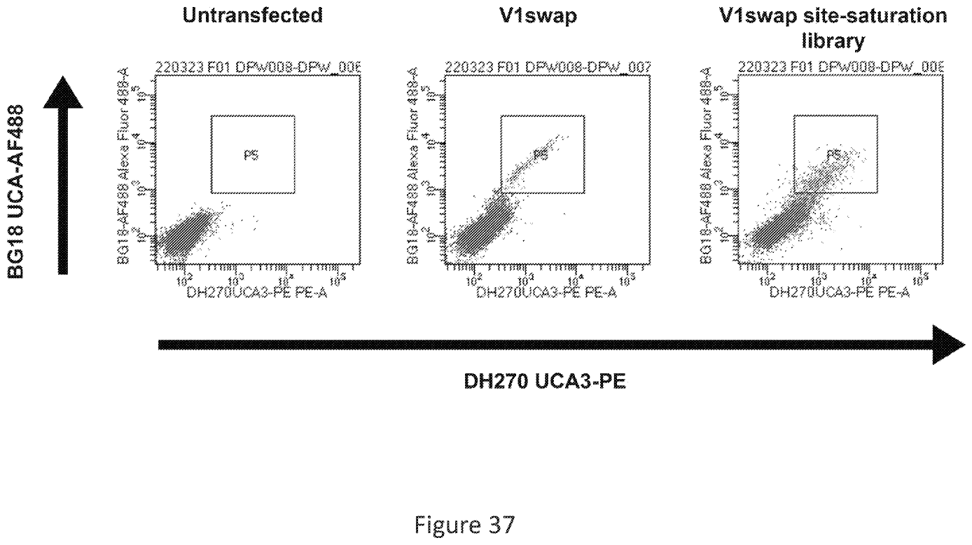

FIG. 33 shows the process of downselection of the gp 160. It first started with mRNA production, in vitro expression and followed by low-dose mRNA-LNP immunizations. The next question was whether the affinity of the V1 chimeric envelope for V3-glycan bnAb precursors can be improved to make a third generation V3-glycan germline-targeting Env. For the third generation V3-glycan germline targeting envelope, the V1 chimeric envelope was tested for improved affinity for V3-glycan bnAb precursors. See FIG. 34. FIG. 35 show that the display library was designed to vary V1-swap residues surrounding the DH270 UCA and BG18 UCA epitopes. FIG. 36 shows the characterization of cell-surfaced expressed V1-swap envelope. FIG. 37 show the verification of expression of V1-swap envelope library.

In summary, for second generation Env immunogens, using structure-based design, we can combine favorable characteristics from CH848 10.17DT and N332-GT5 to generate a single immunogen that is capable of binding to precursors from multiple V3 glycan lineages. The second generation Env immunogen has been designed as stabilized gp 160s for production as an mRNA vaccine. For third generation Env immunogens, mammalian cell display can be used to both increase the affinity of BG18 and DH270 UCAs for the Env and to expand reactivity to additional V3-glycan UCAs. Precursor frequency of V3-glycan bnAbs is expected to be low. Therefore, the second generation CH848 envelope could increase the probably of eliciting V3-glycan neutralizing antibodies by binding to multiple distinct types of V3-glycan bnAb precursors. The previously results support that stabilized gp 160s are an Env format that is amenable to expression by mRNA. For optimal immunogenicity, high expression will be beneficial.

### Example 3

This example describes animal studies with HIV-1 envelopes designed to prime and boost V3 glycan antibodies lineages.

The envelopes described in Table 6, expressed as recombinant proteins or modified mRNA formulated in LNP, are analyzed in animal studies including mouse and NHP animal models. The mouse animal model could be any model, including an animal model comprising a DH270UCA transgene.

Any suitable adjuvant will be used.

The envelopes in Table 6 will be produced under cGMP conditions as a recombinant protein and/or mRNA formulated in LNP for use in Phase I clinical trial.

### Example 4

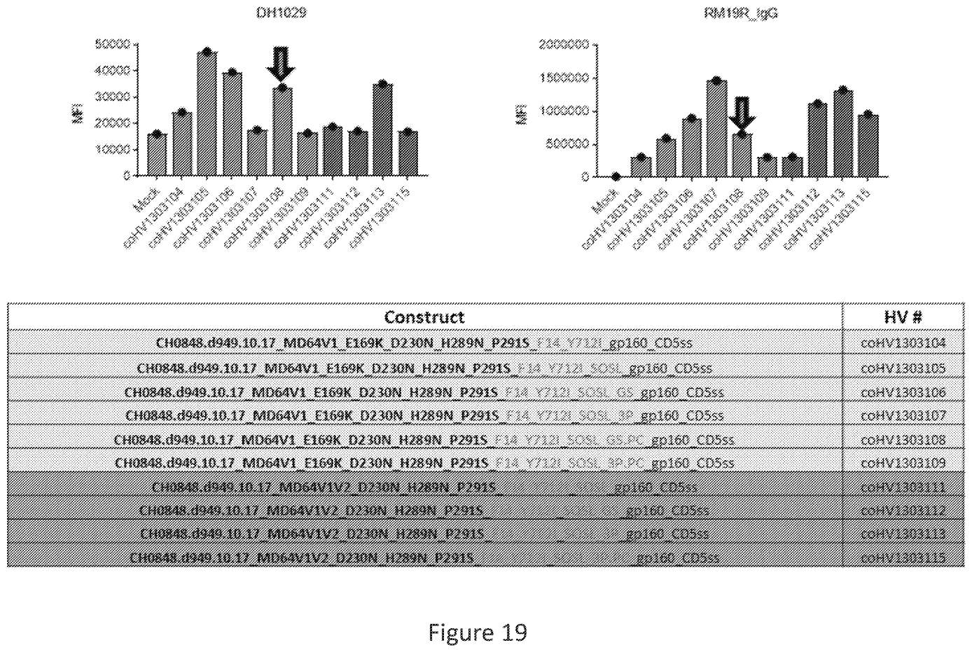

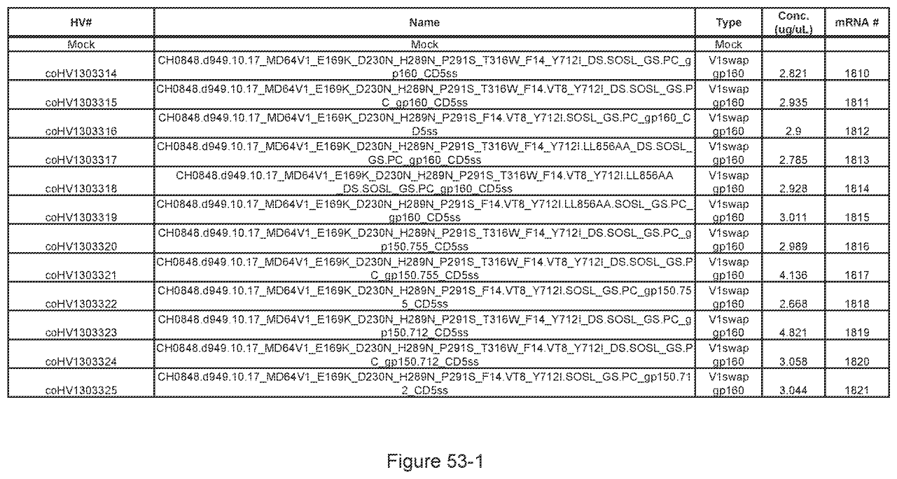

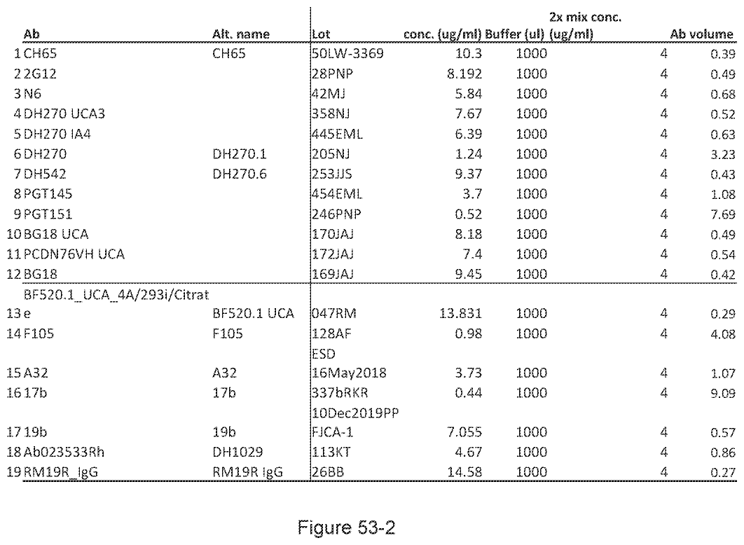

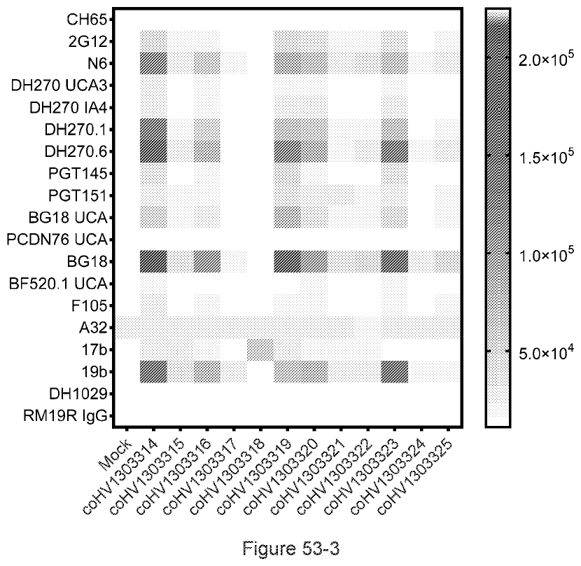

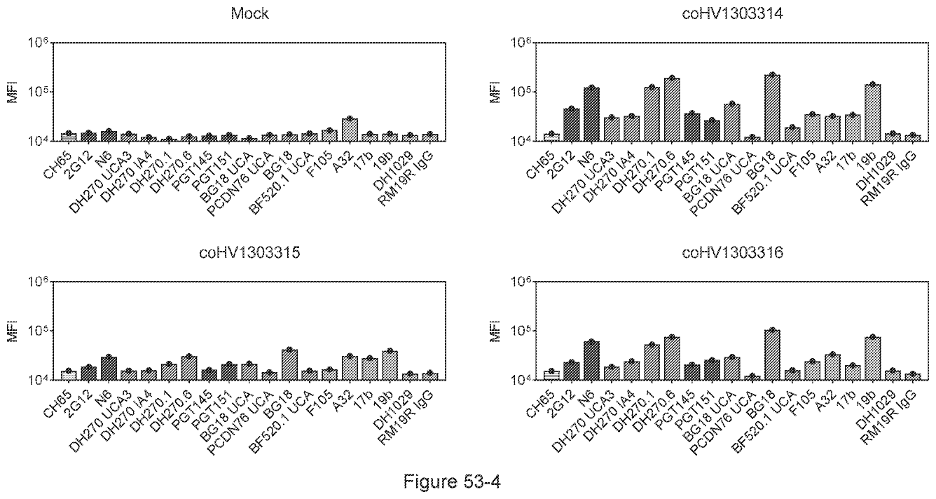

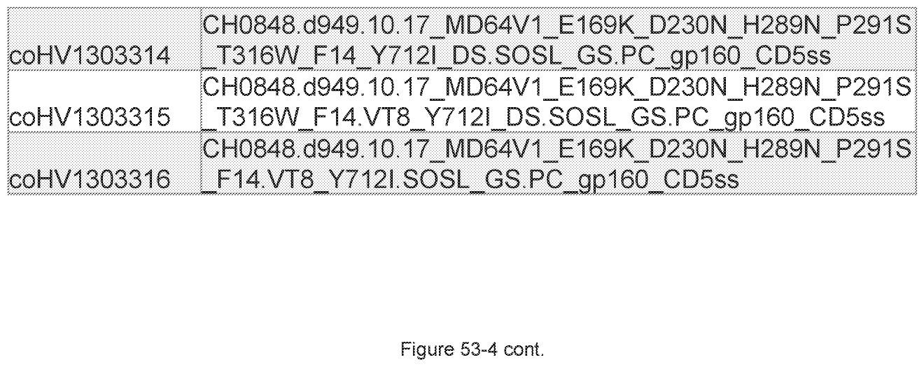

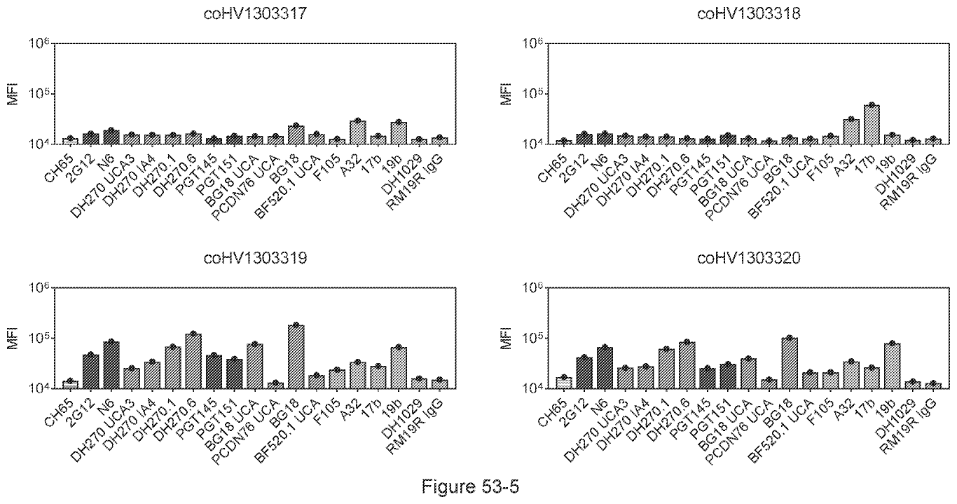

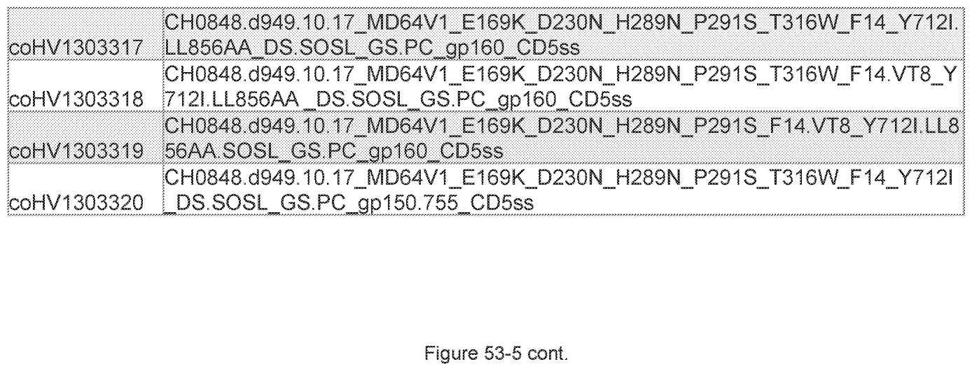

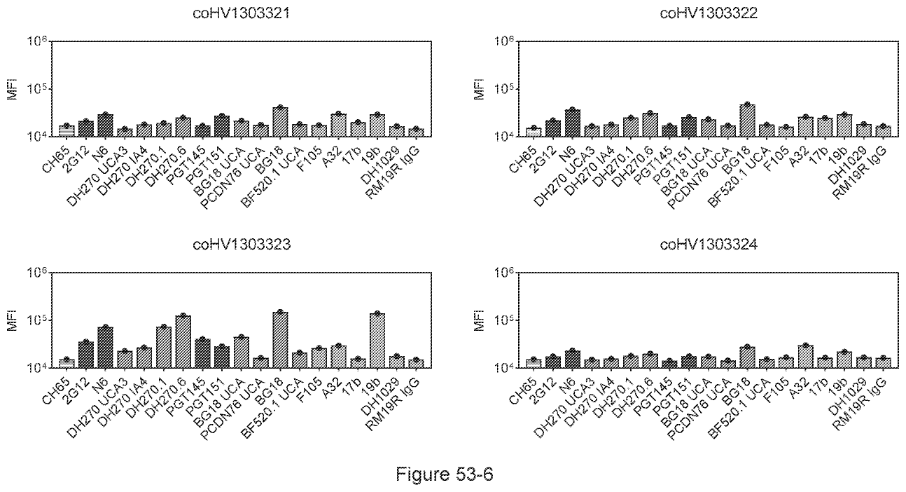

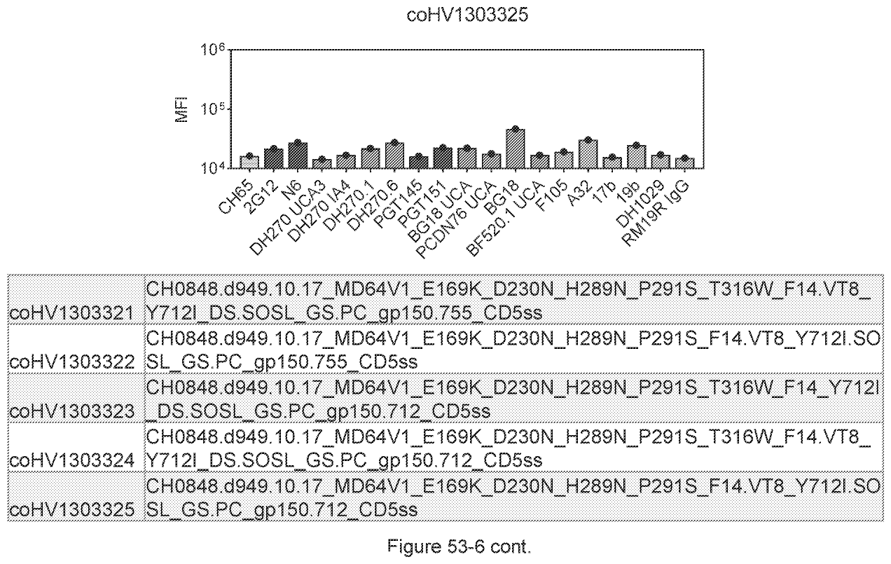

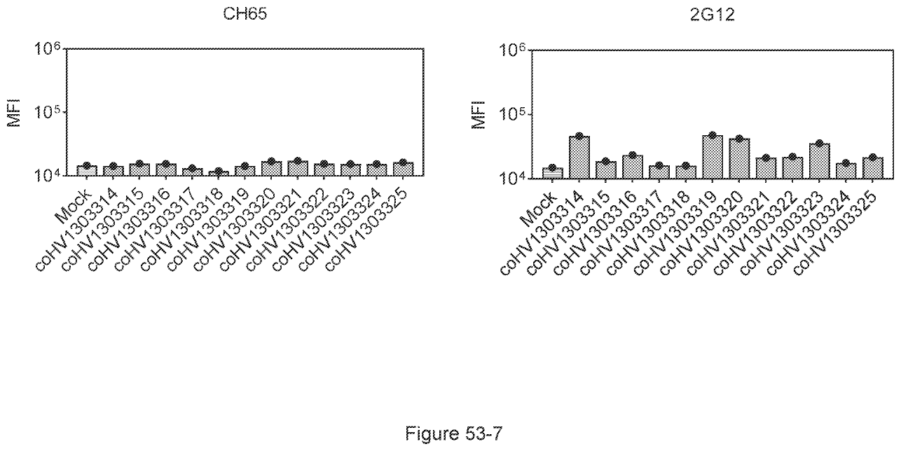

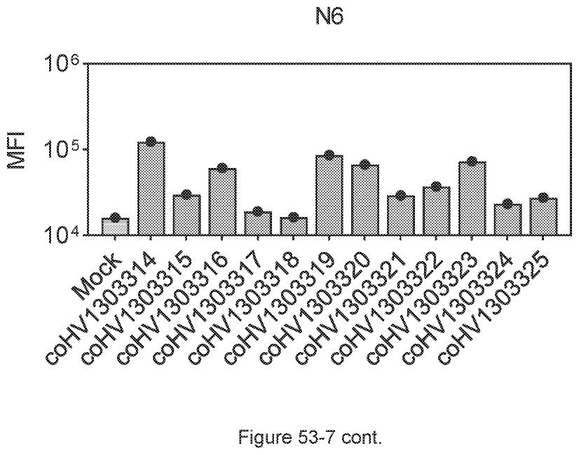

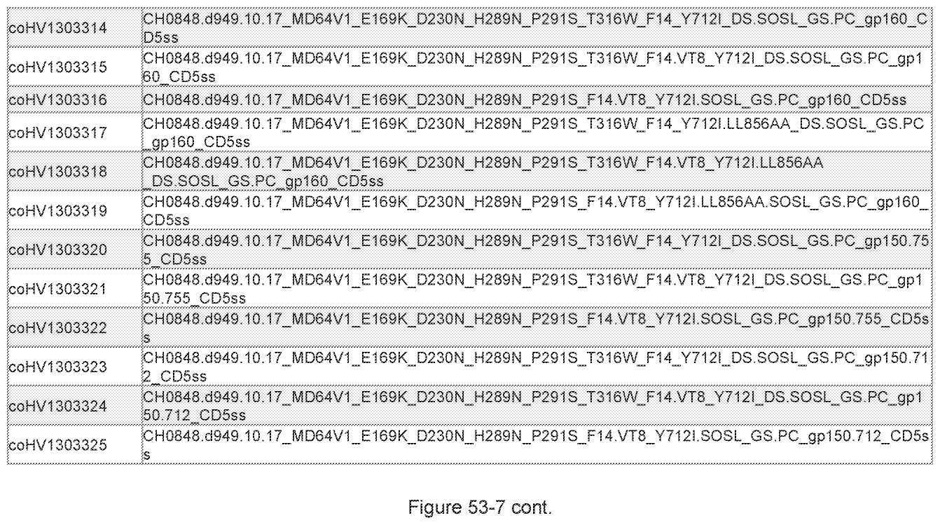

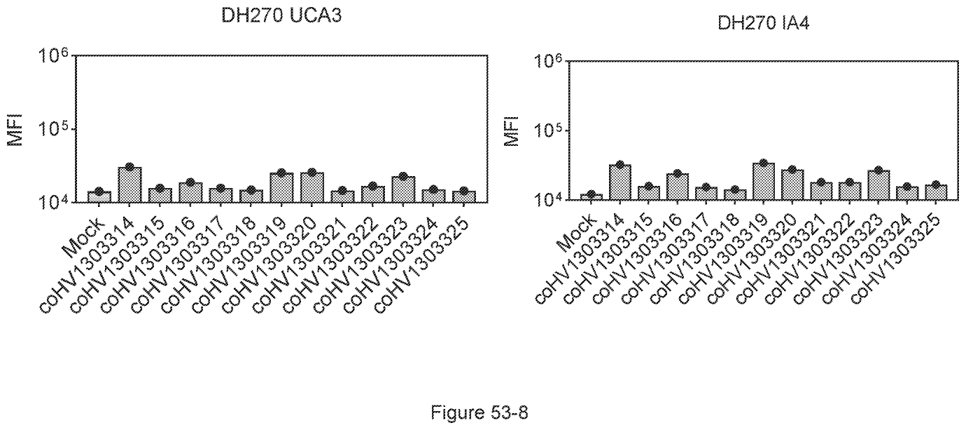

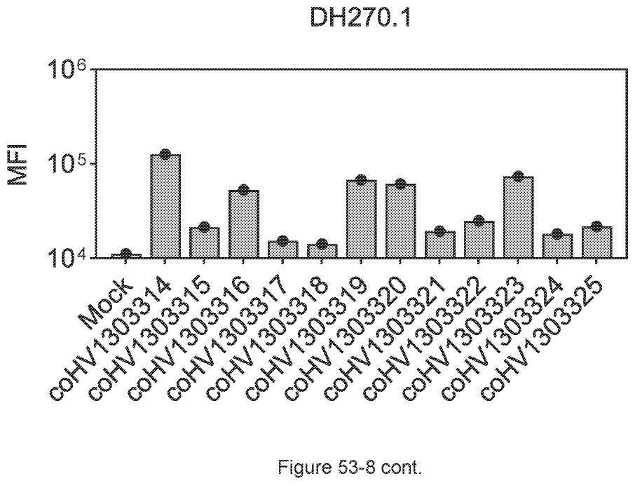

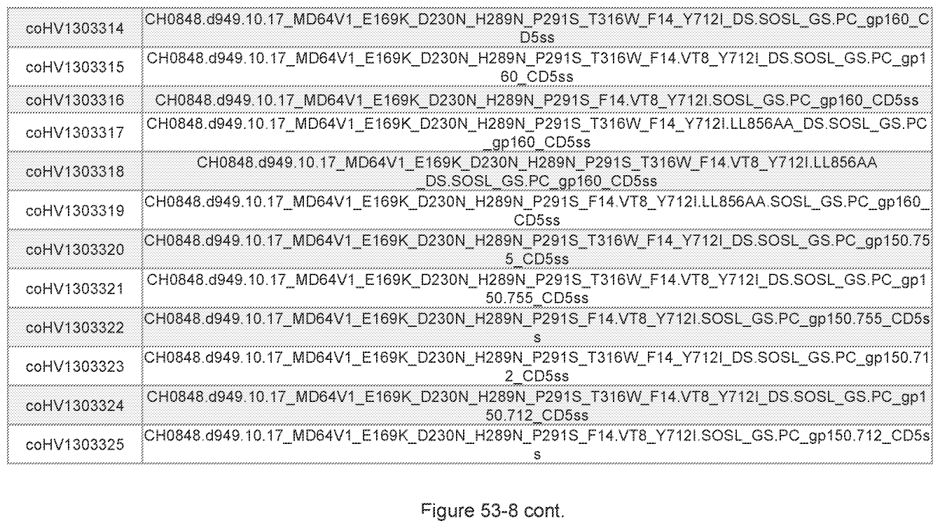

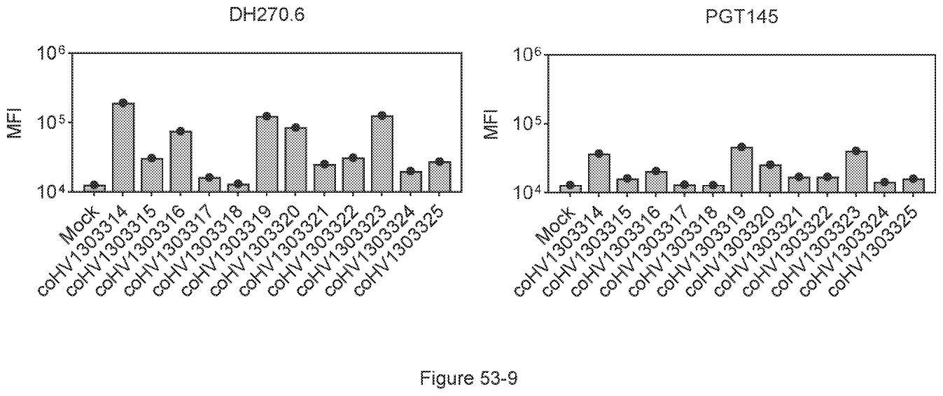

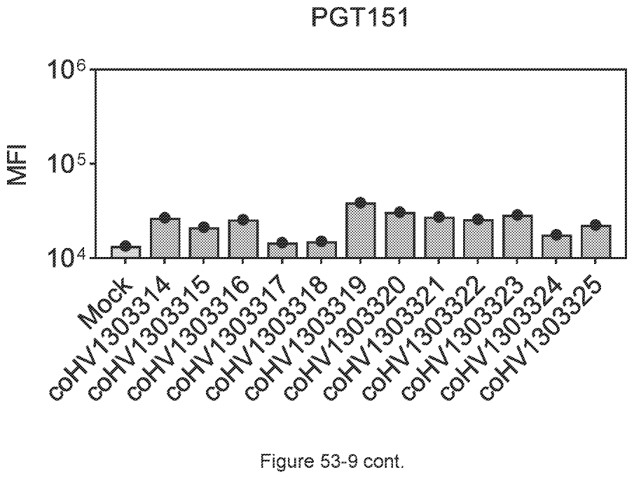

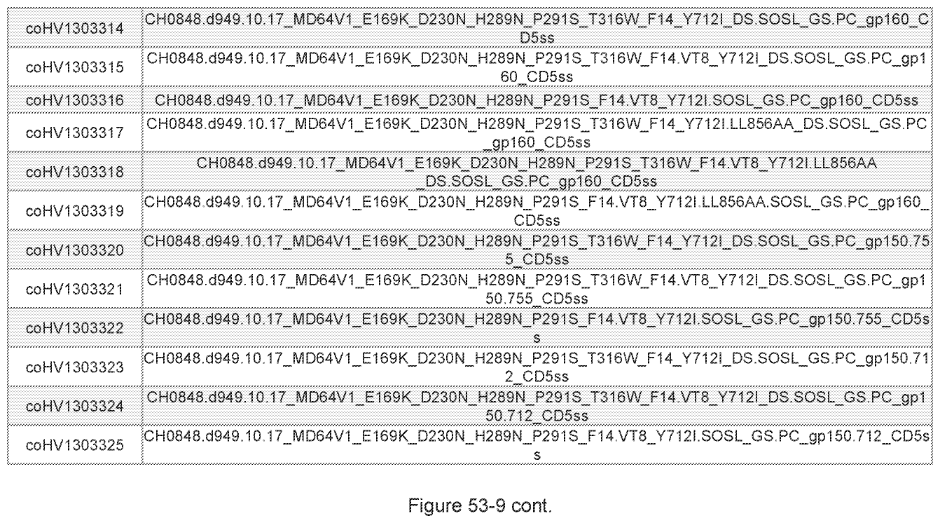

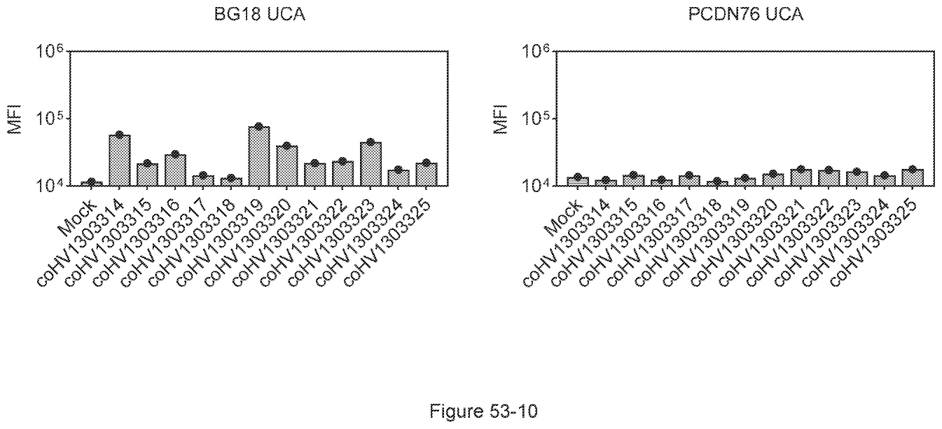

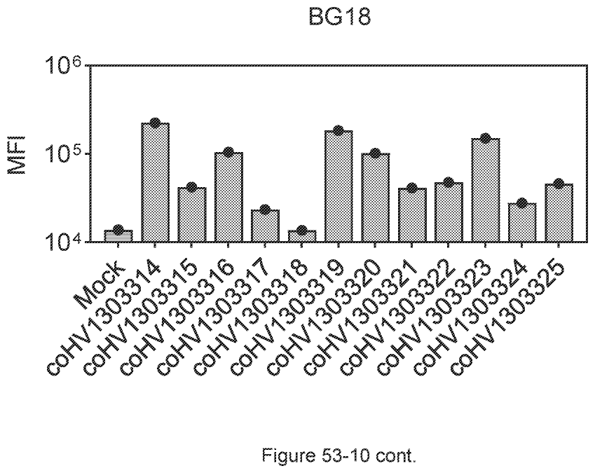

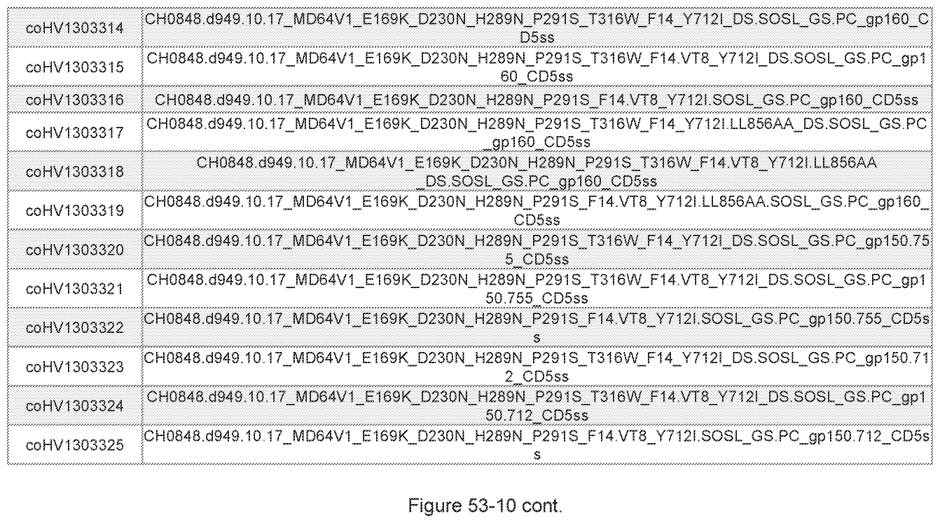

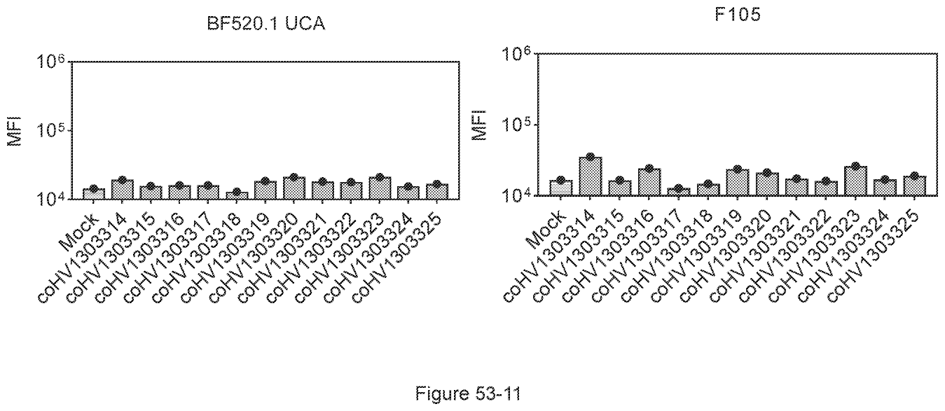

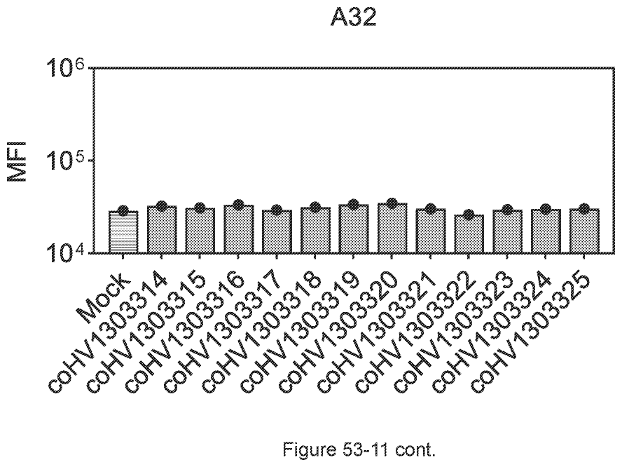

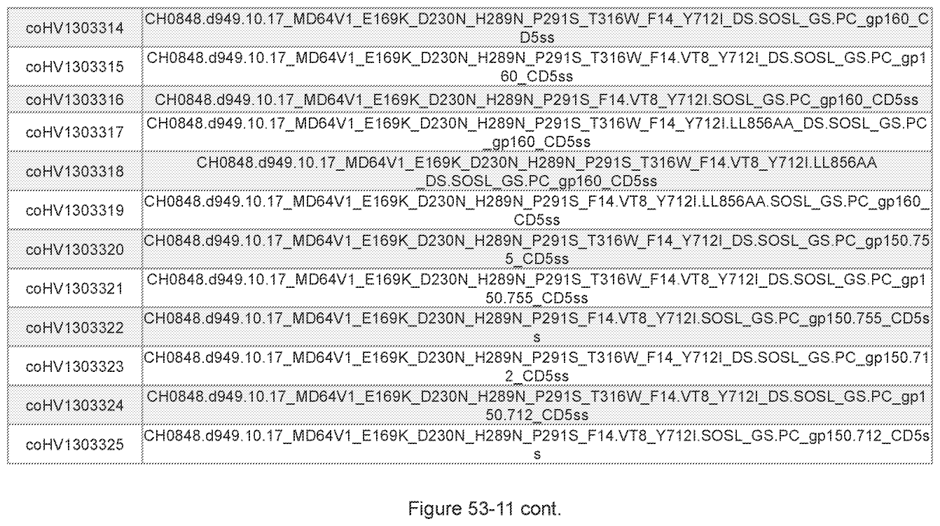

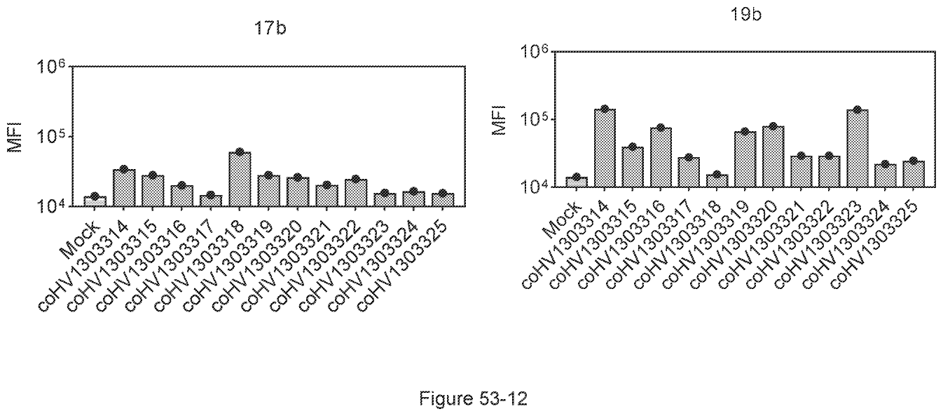

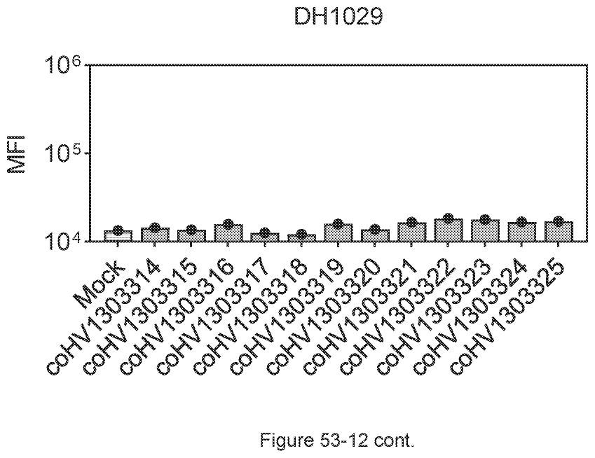

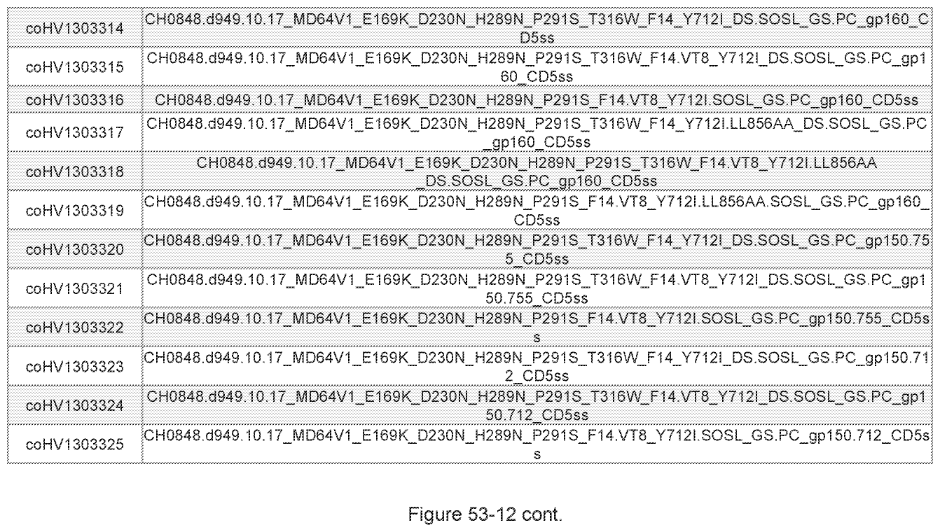

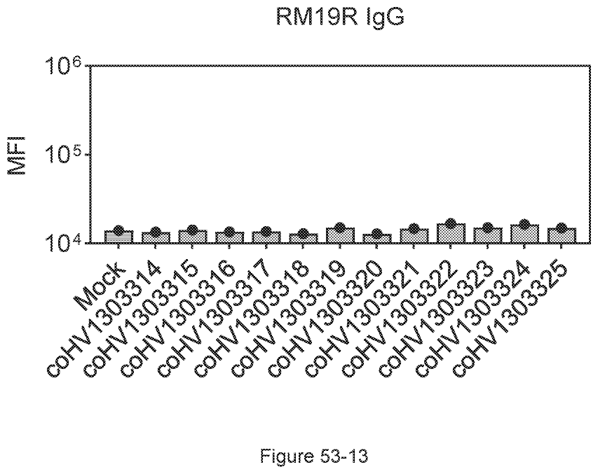

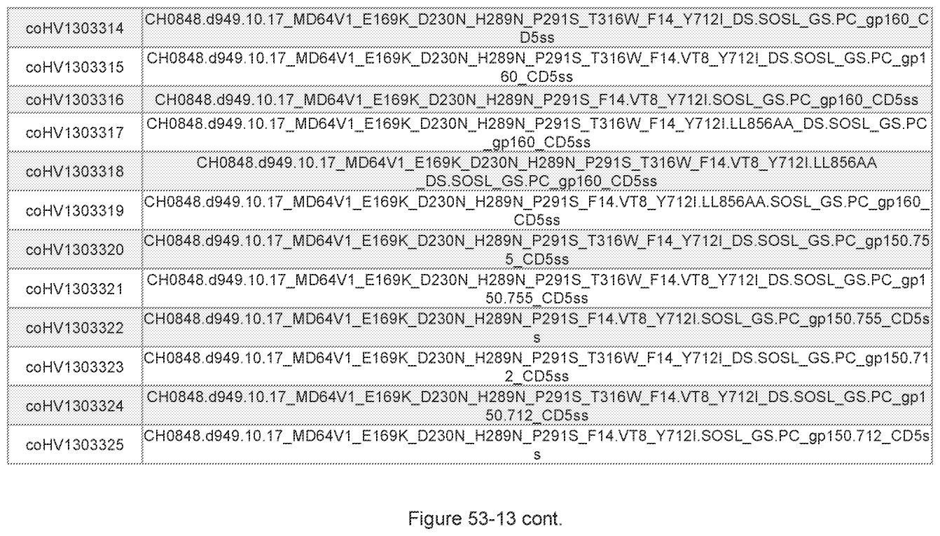

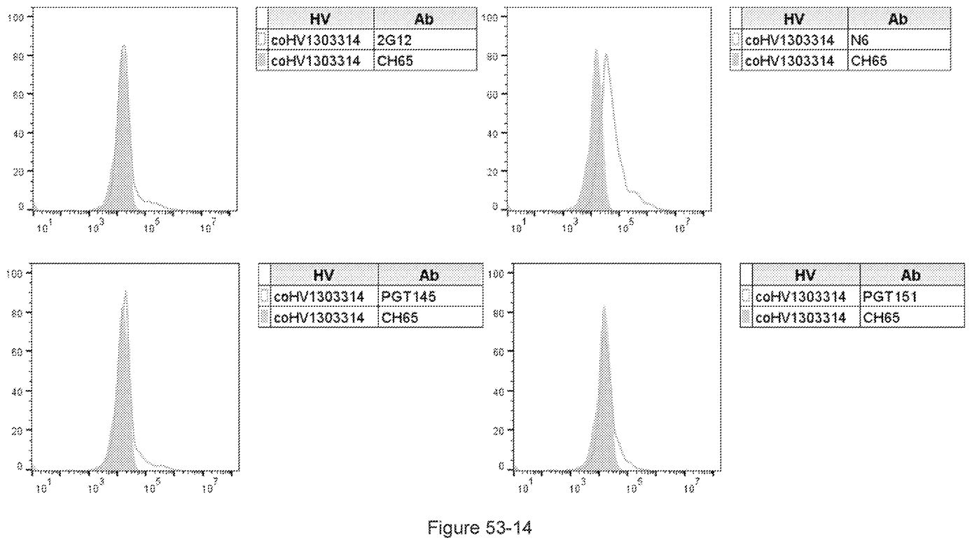

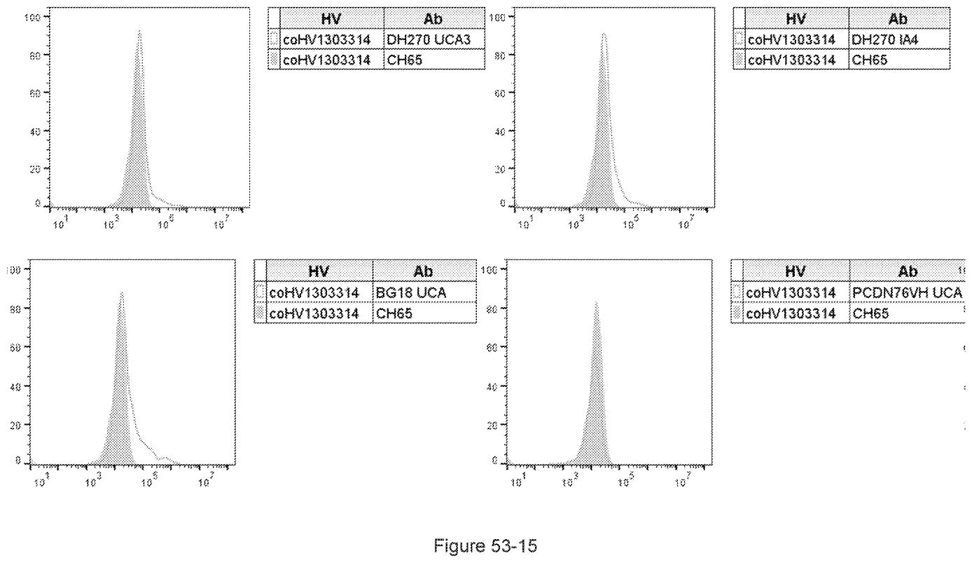

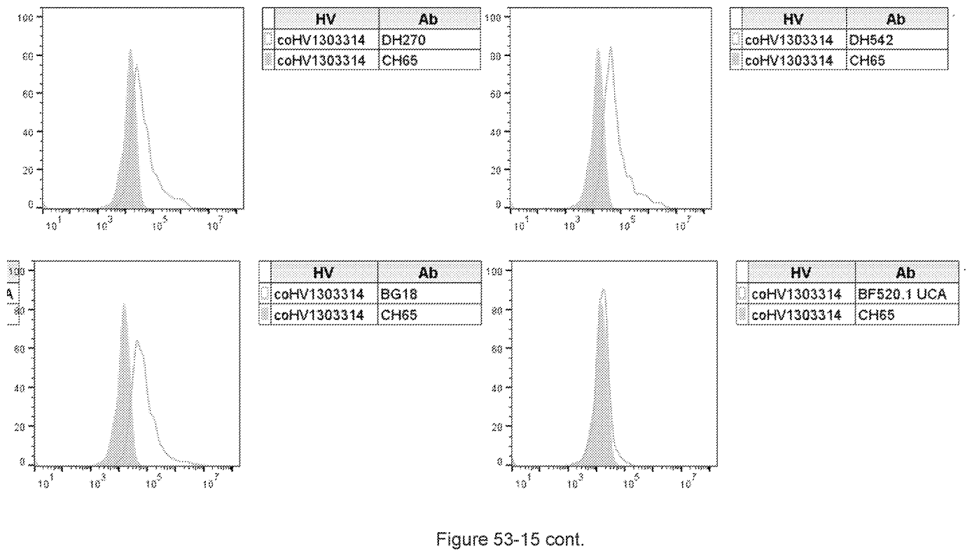

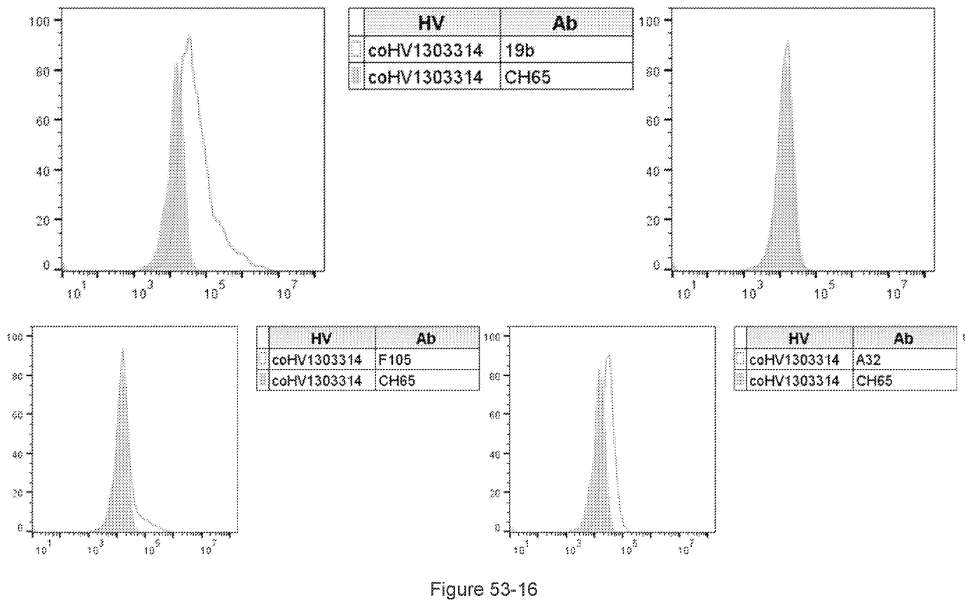

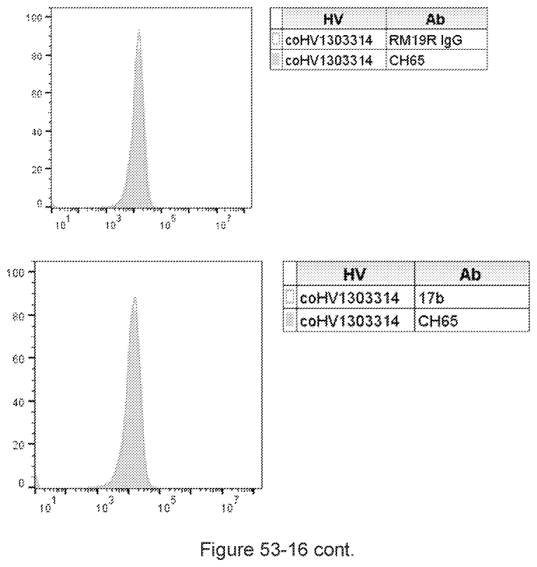

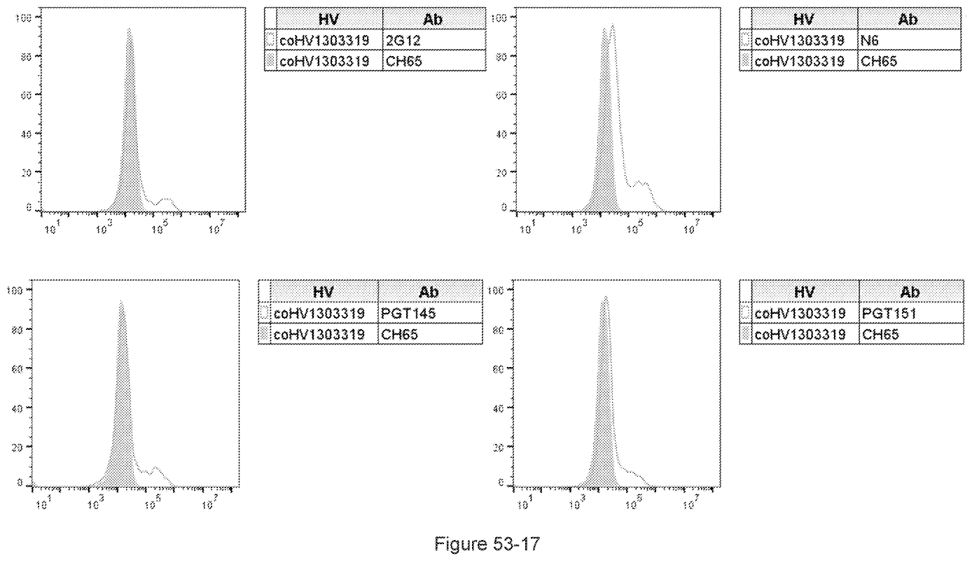

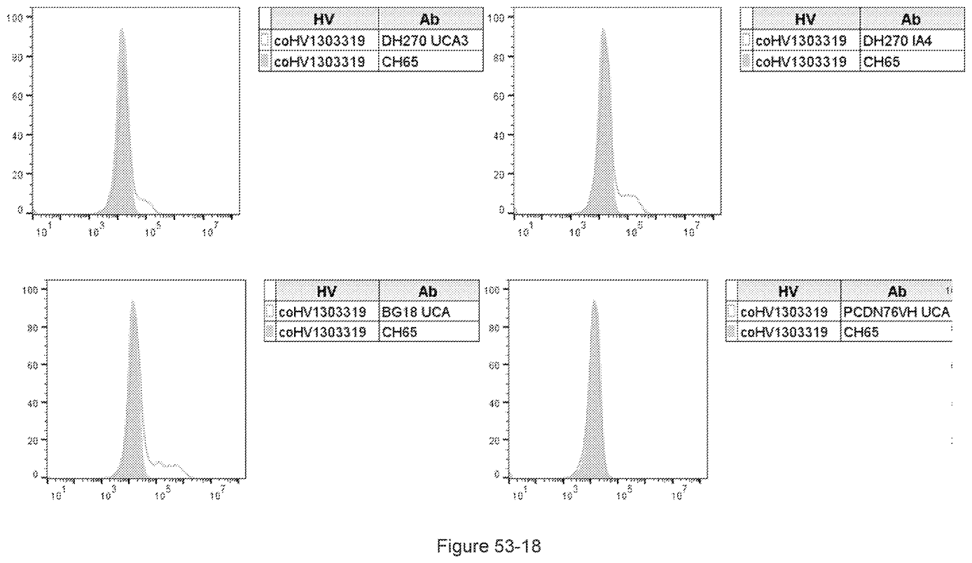

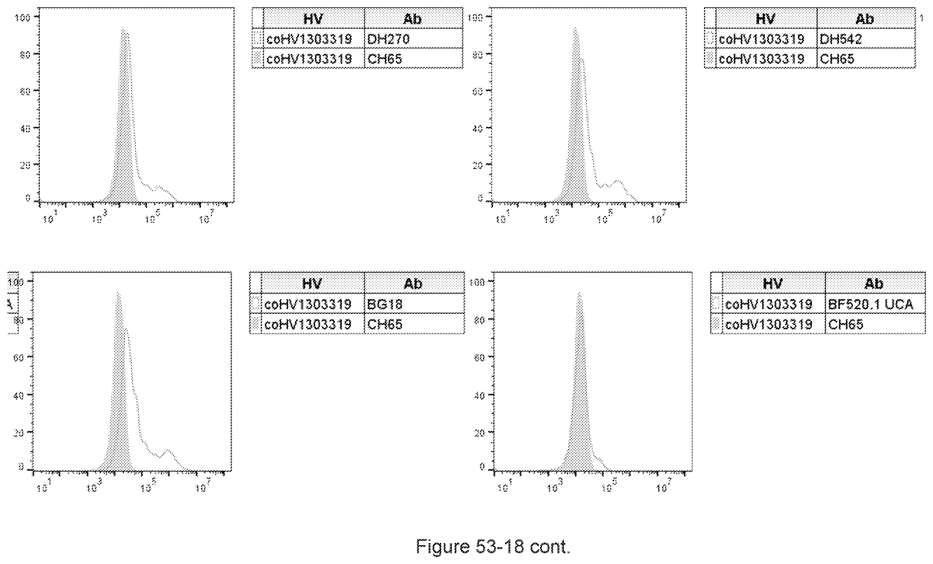

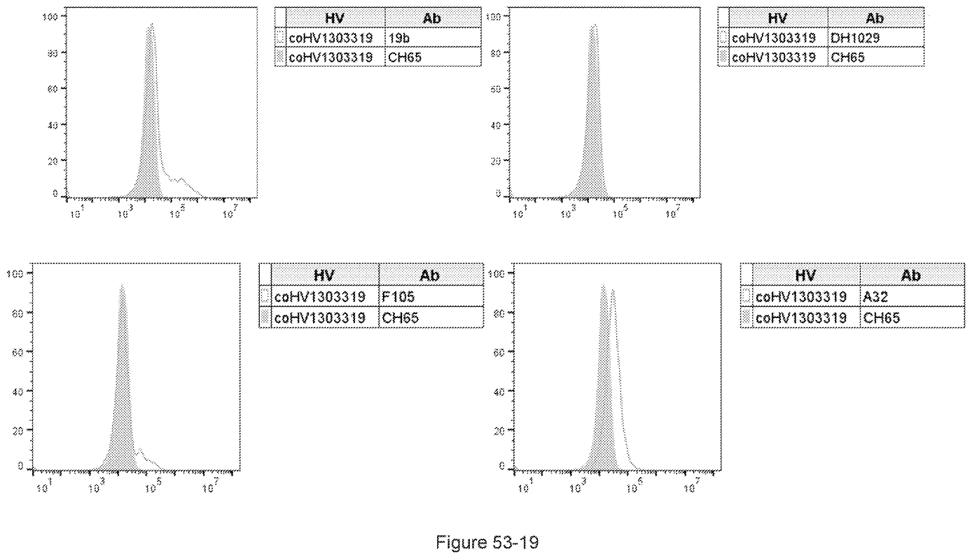

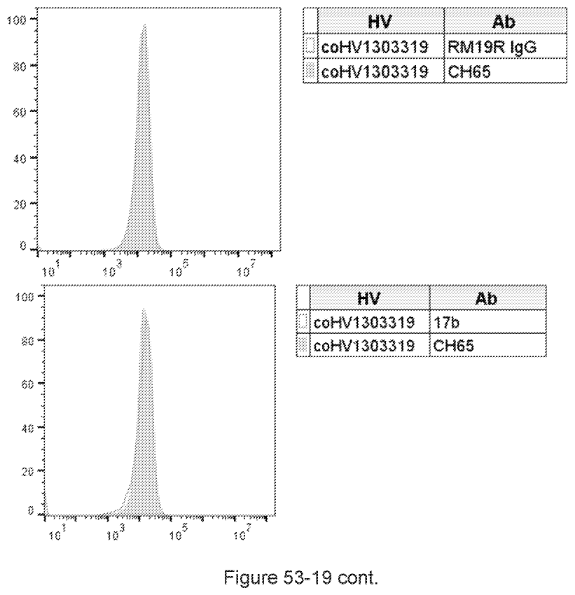

FIG. 53 parts 1-19 show additional CH848 V1swap designs (coHV130314-coHV130325). FIG. 53-2 shows a list of tested antibodies. FIG. 53-3 shows a heatmap. FIGS. 53-4-53-13 show the binding reactions of coHV130314-coHV130325. FIGS. 53-14-53-19 show relative flow cytometric histogram of coHIV130314 and coHV130319.

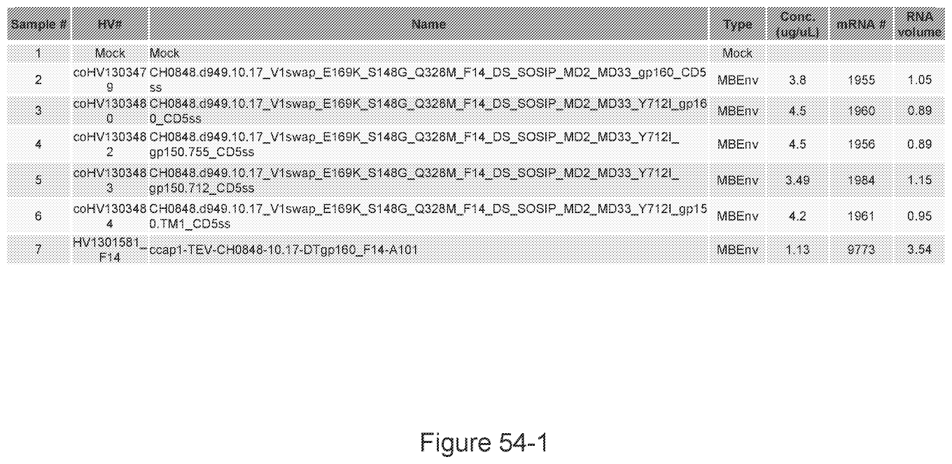

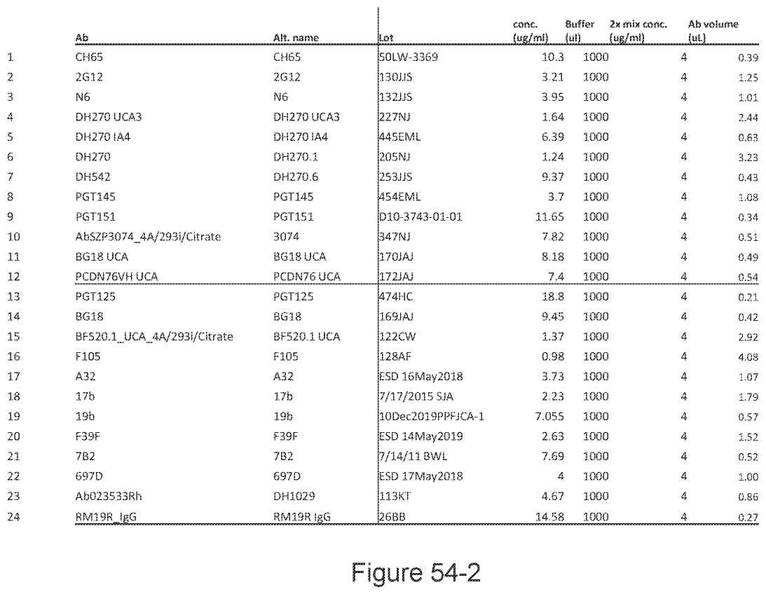

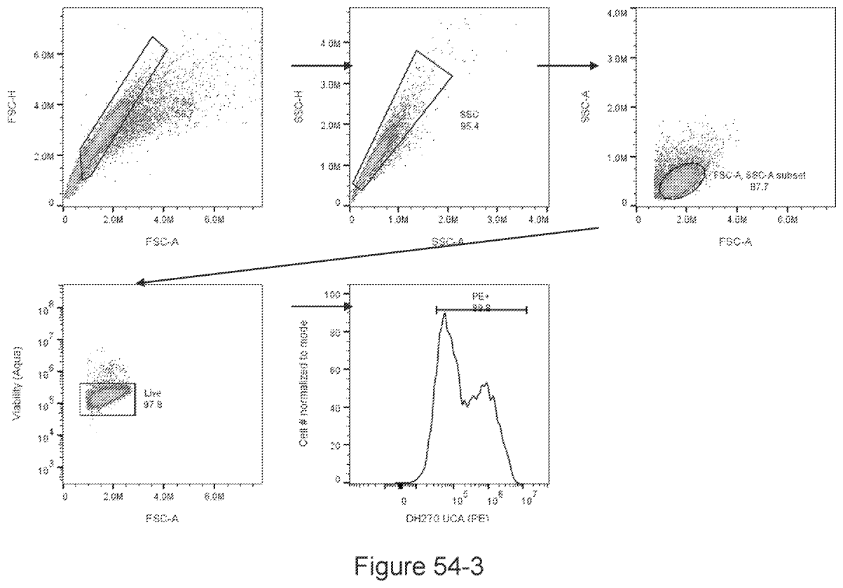

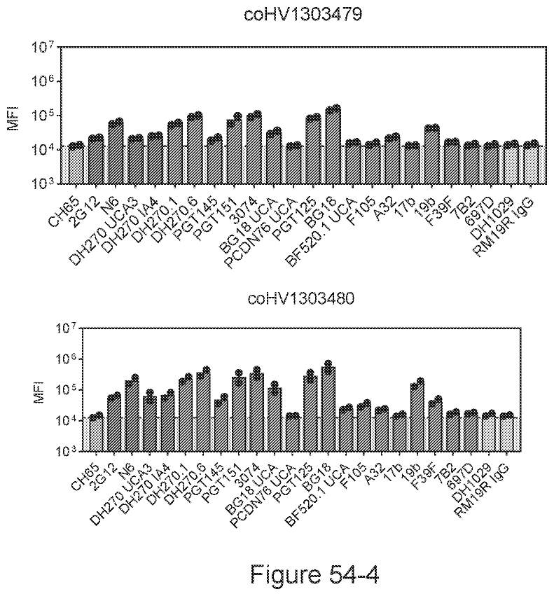

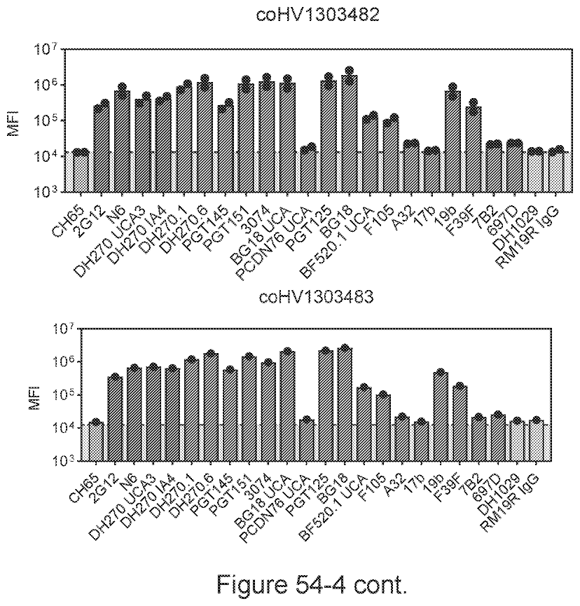

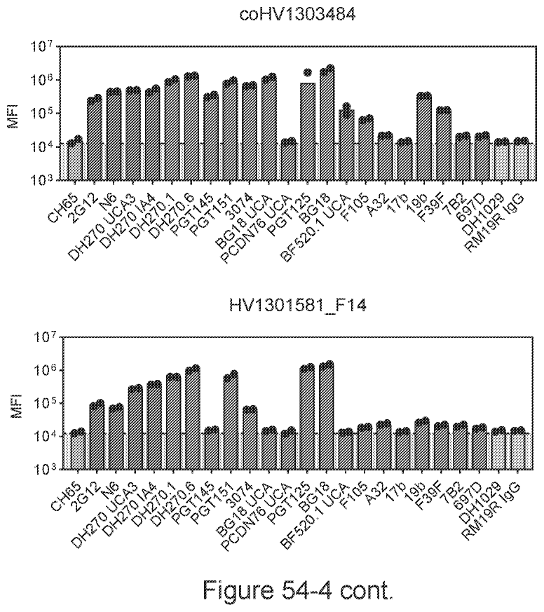

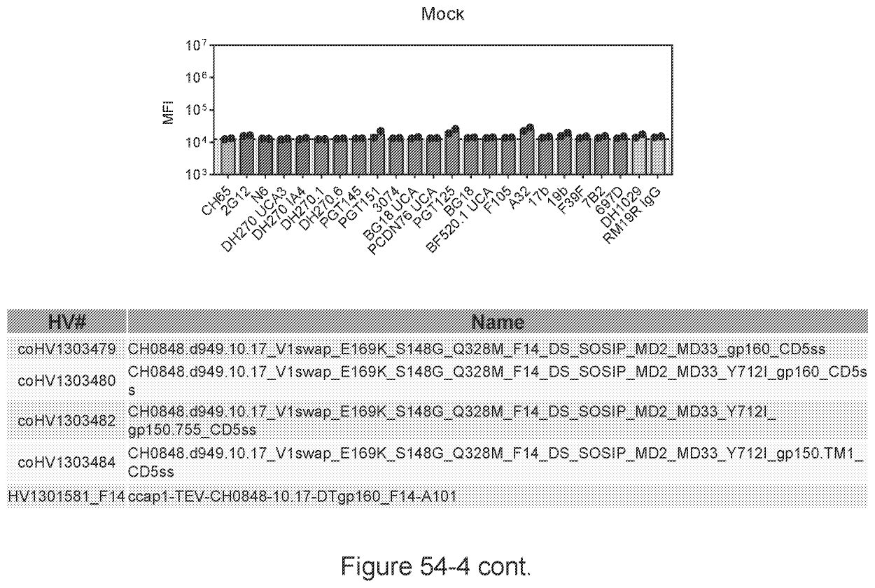

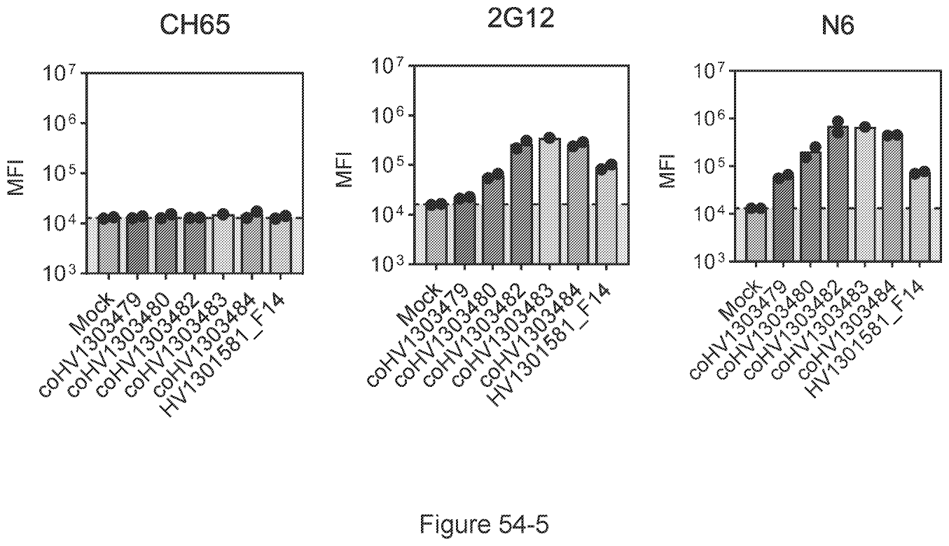

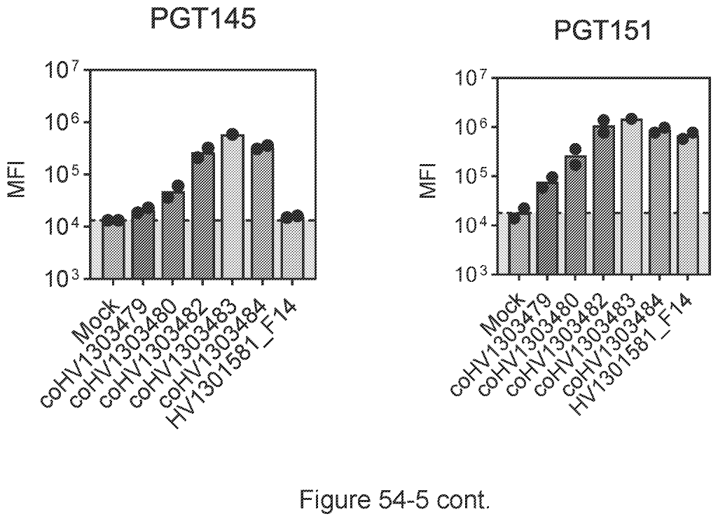

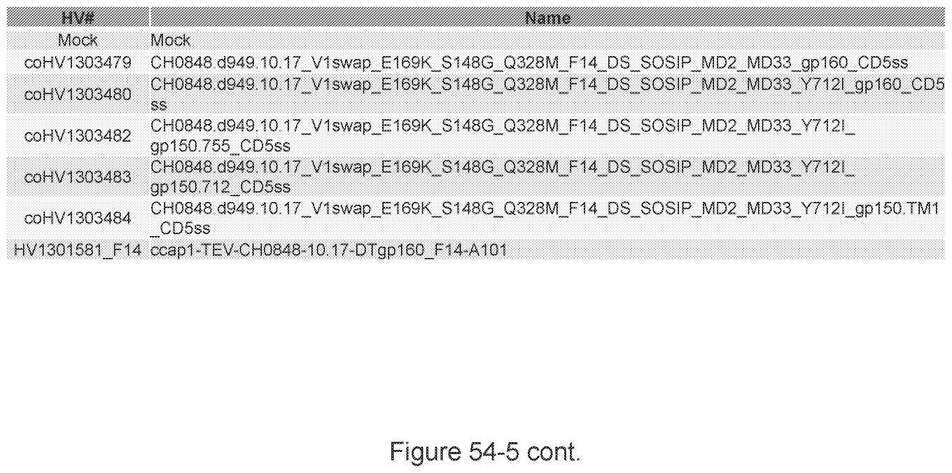

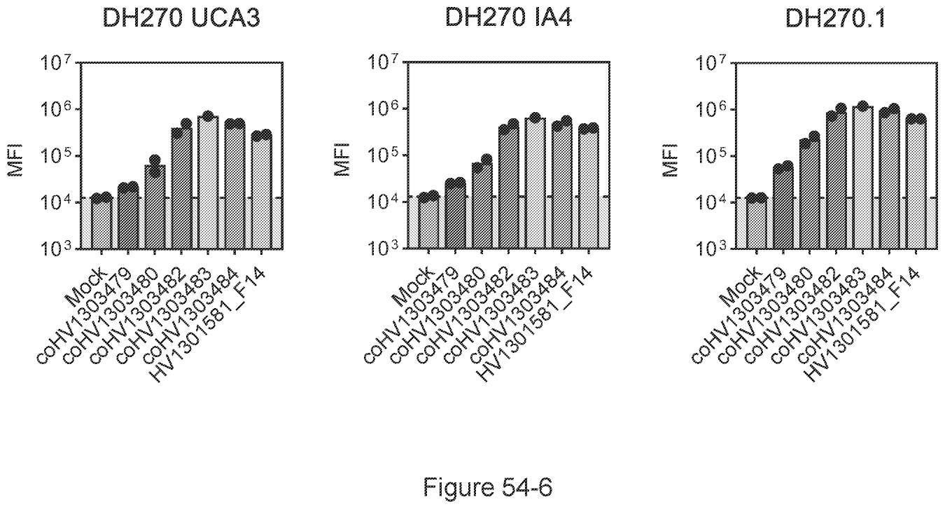

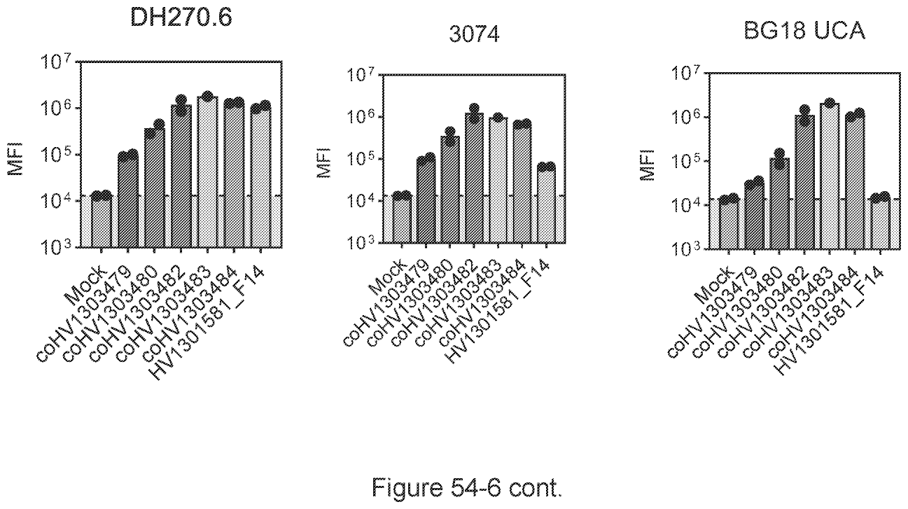

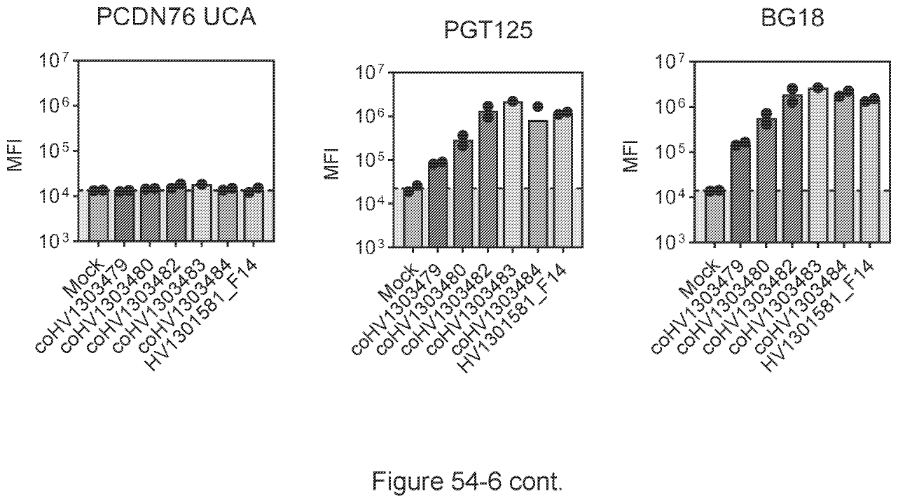

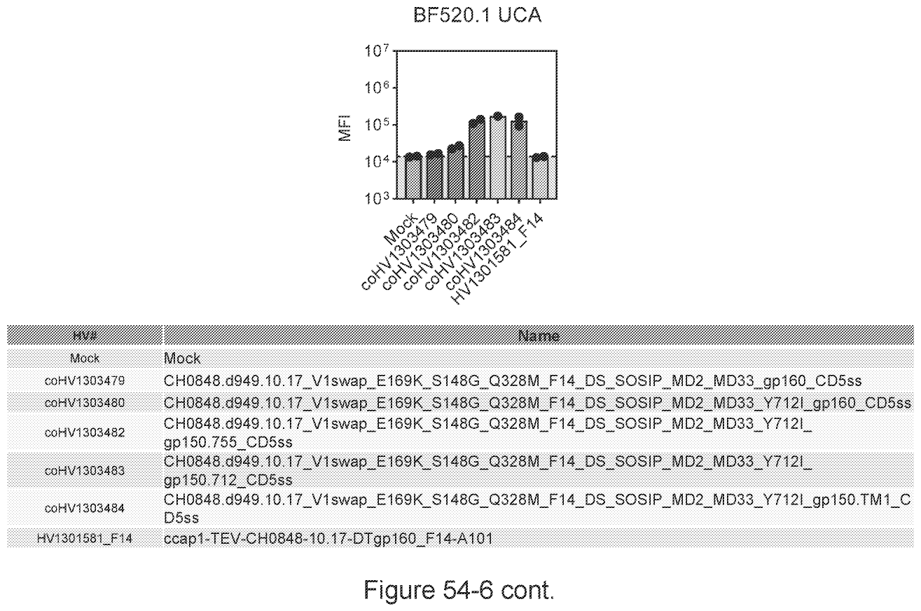

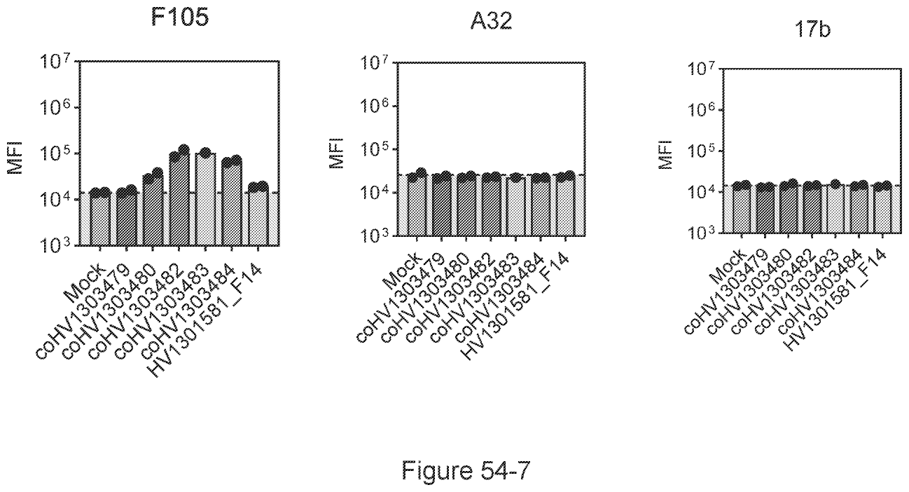

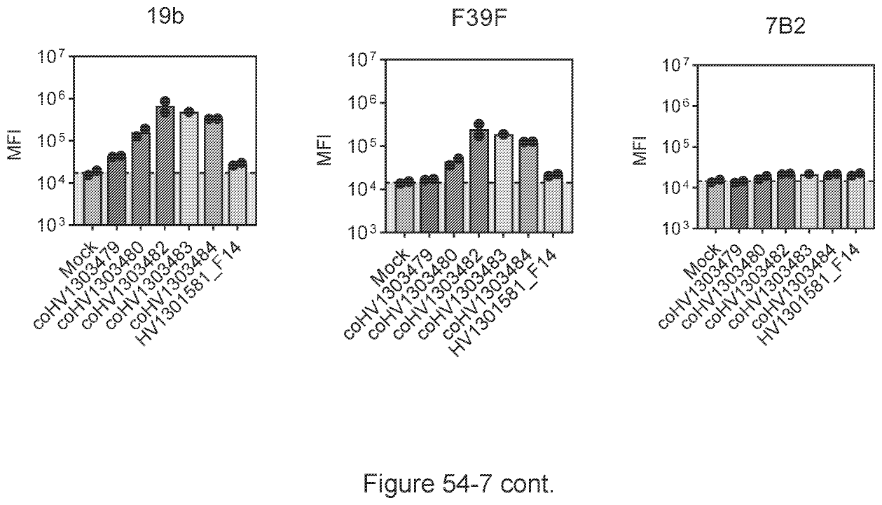

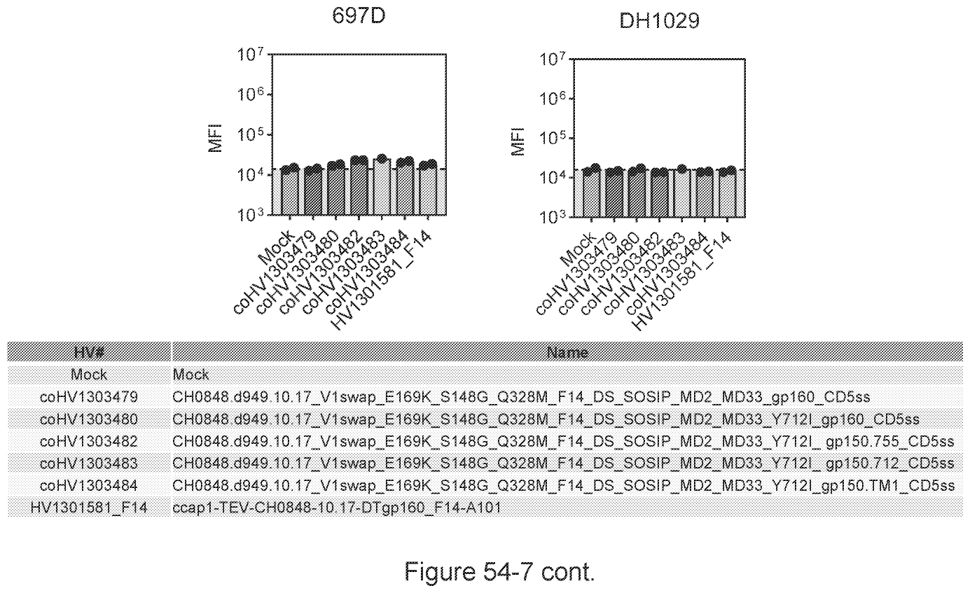

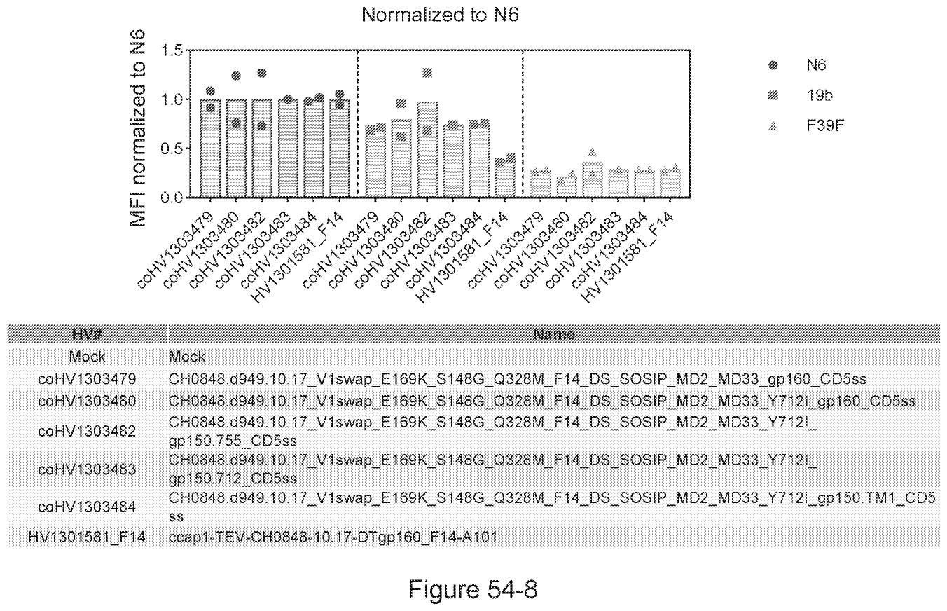

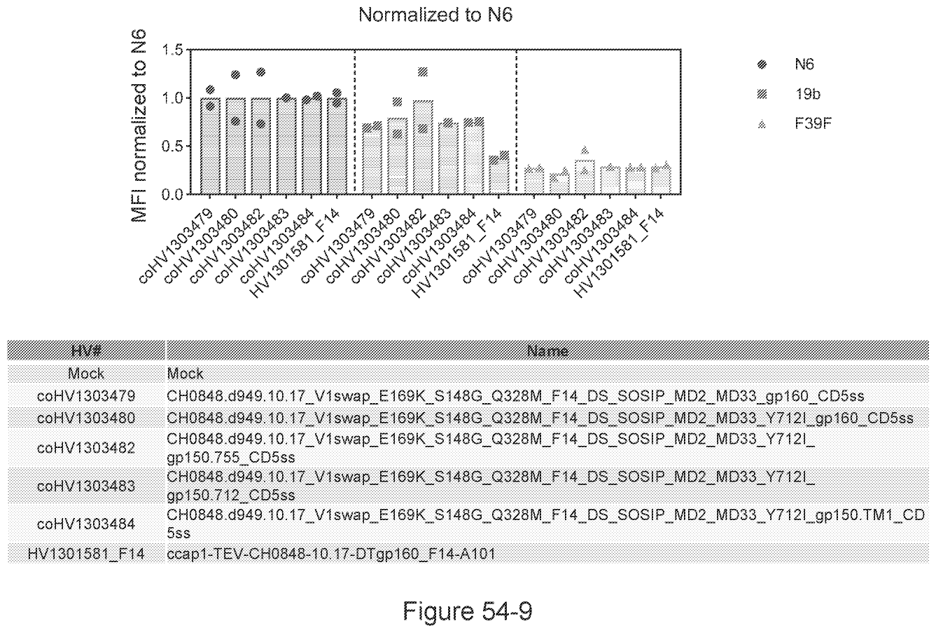

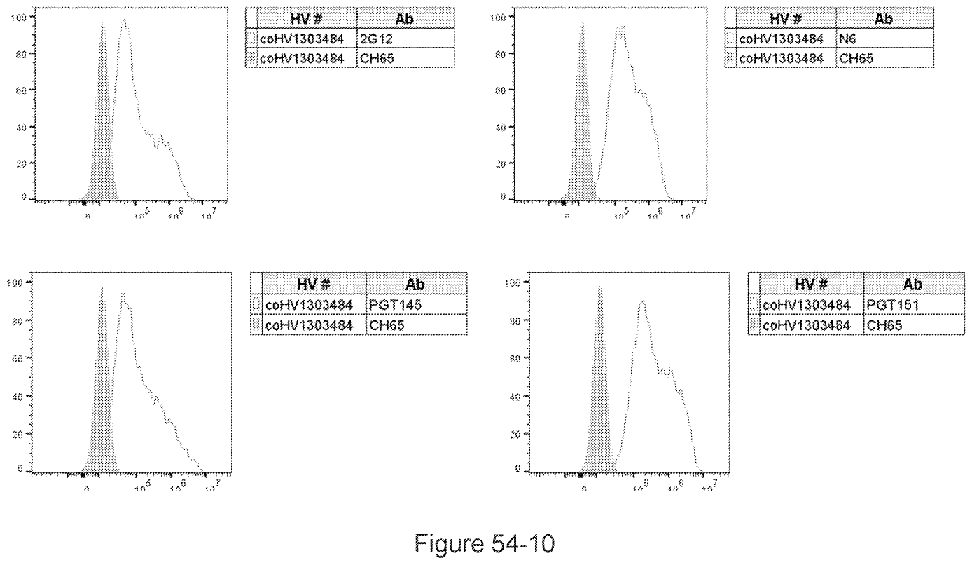

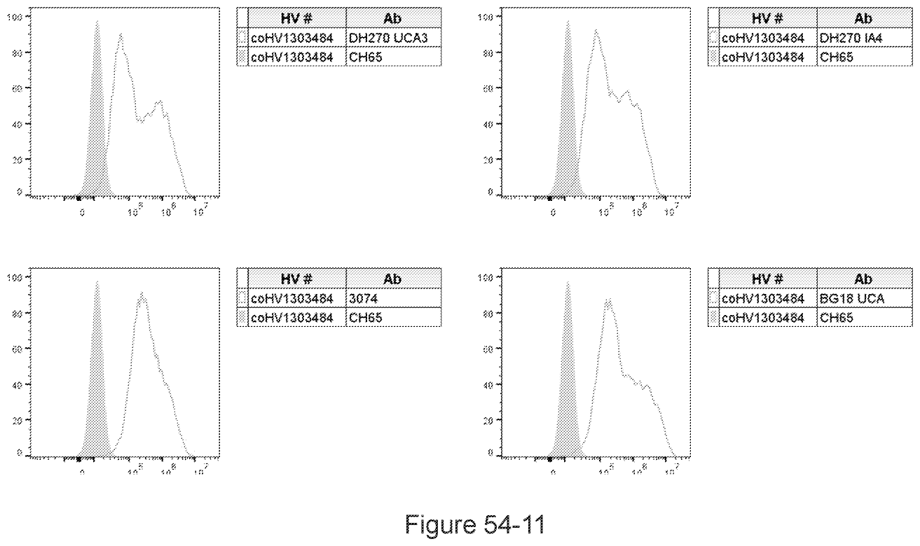

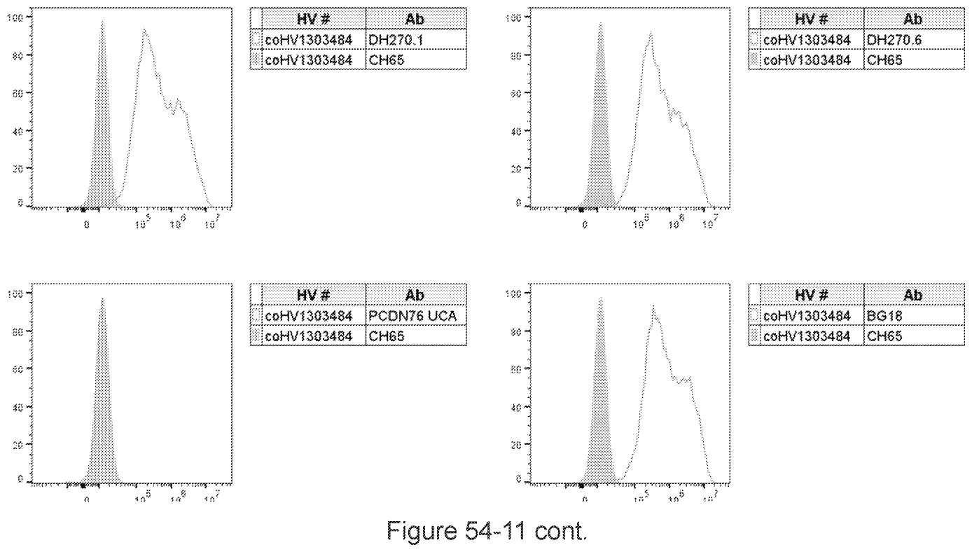

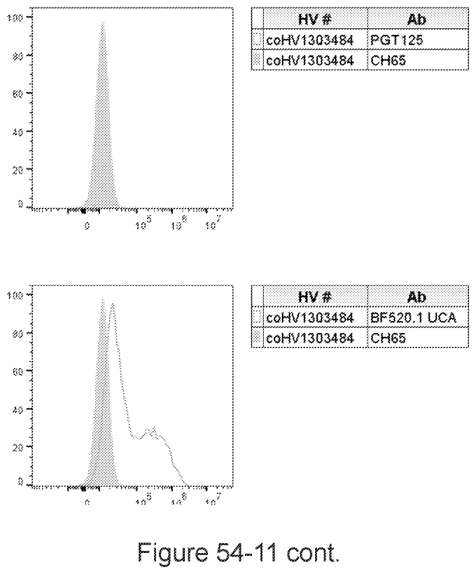

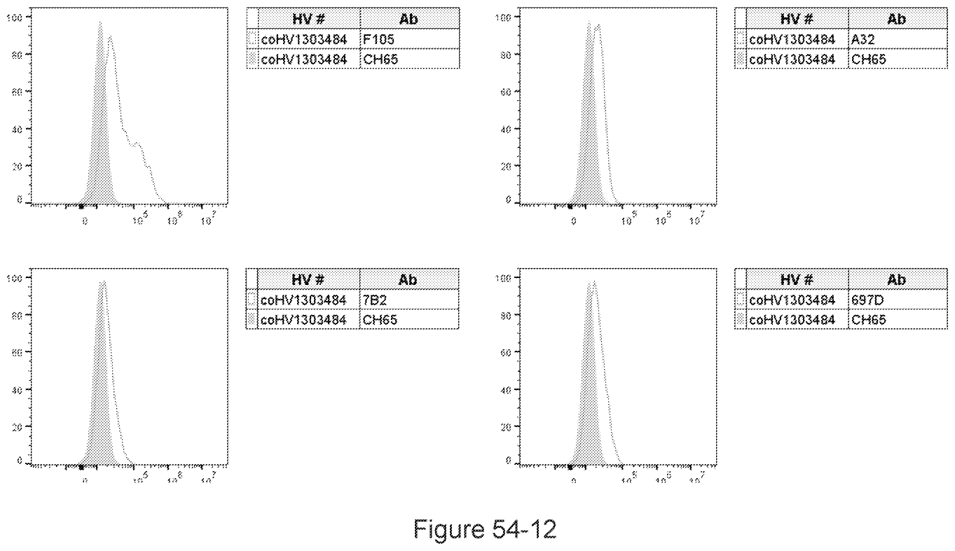

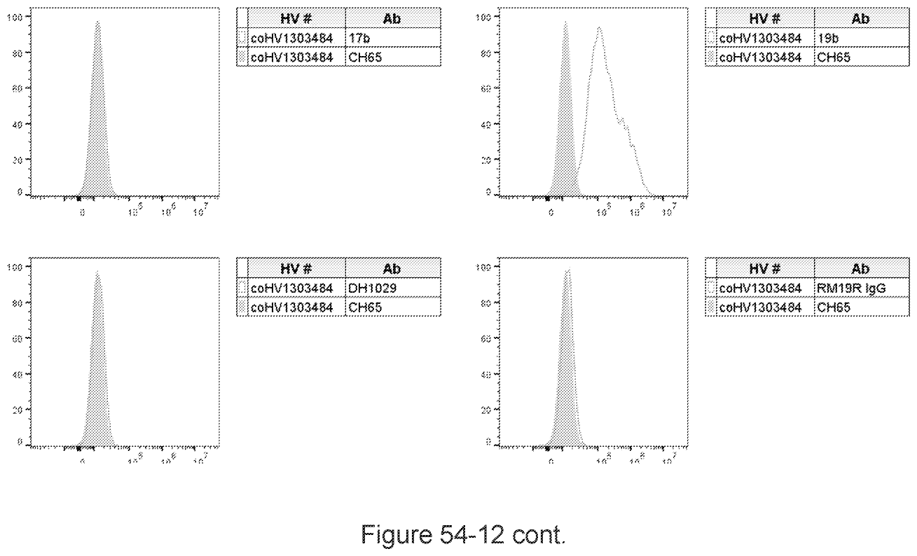

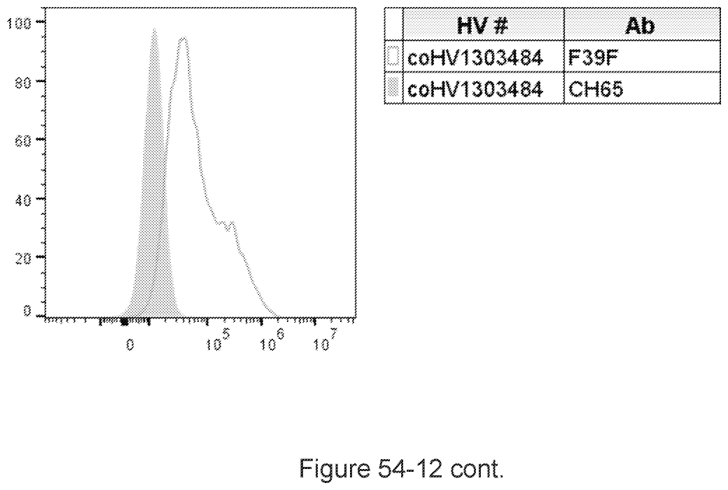

FIG. 54 parts 1-12 show V1swap E169K S148G_Q328M membrane-bound Envs expressed from mRNAs (coHIV1303479, coHV1303480, coHV1303482-coHV1303484 and HV1301581_F14). FIG. 54-2 shows a list of tested antibodies. FIG. 54-3 shows the gating data of V1 swap E169K S148G_Q328M membrane-bound Env designs. FIGS. 54-4-54-9 show the binding reactions of Envs coHV1303479, coHV1303480, coHV1303482-coHV1303484 and HV1301581_F14. FIGS. 54-10-54-12 show relative flow cytometric histogram of coHV1303484.

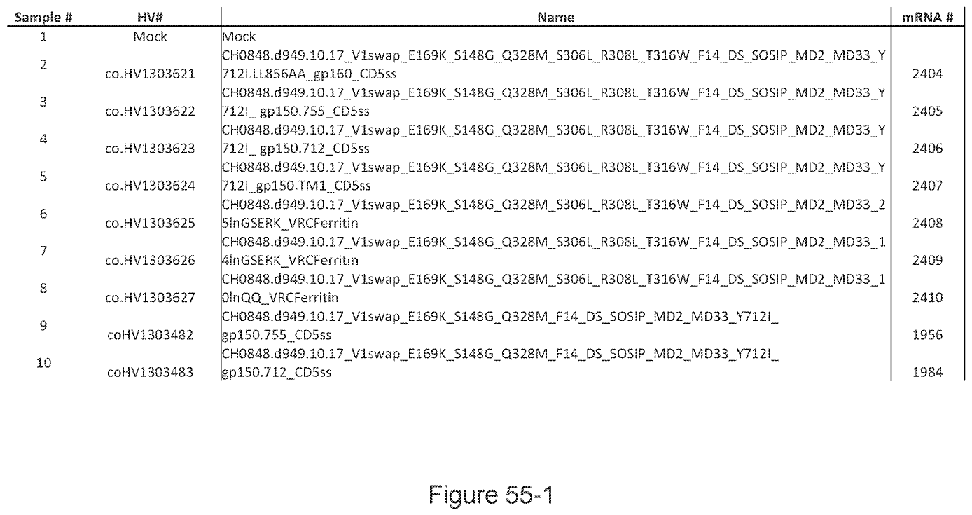

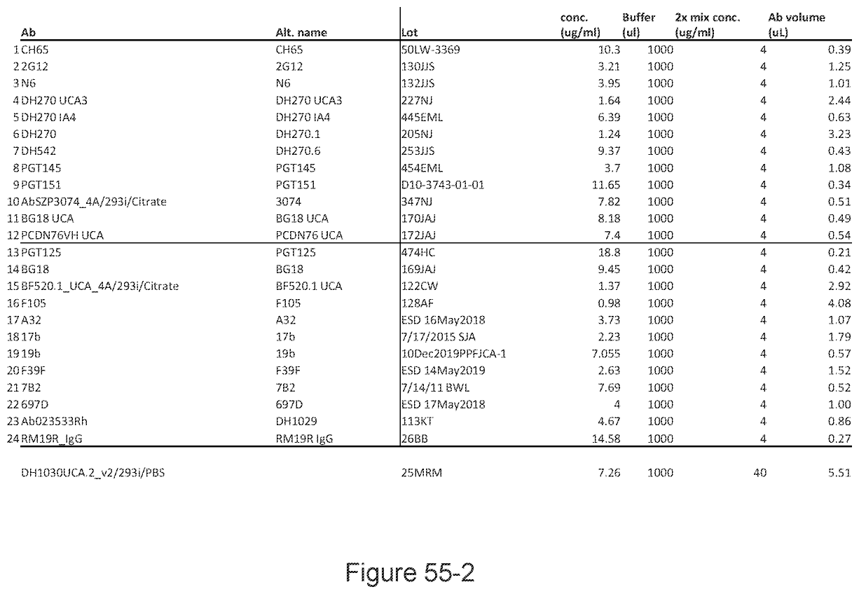

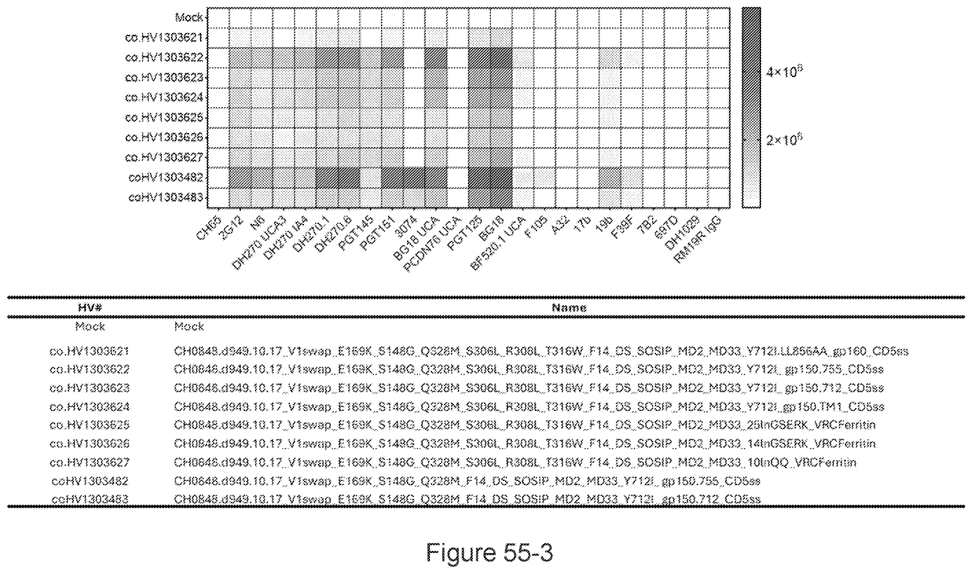

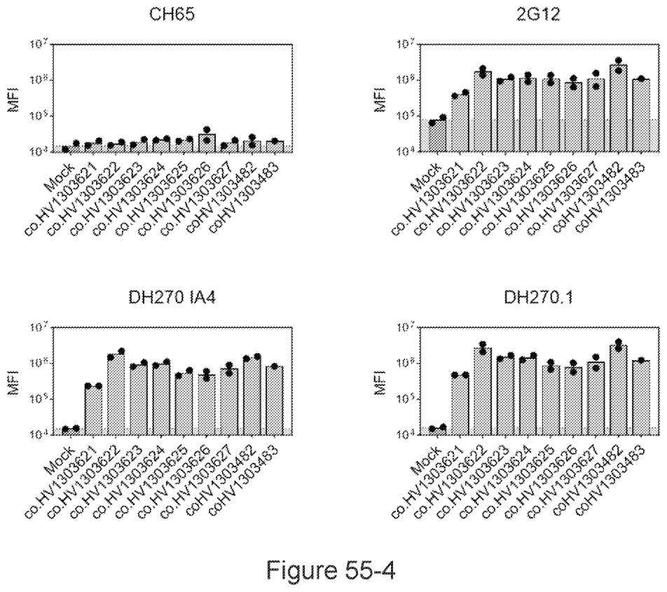

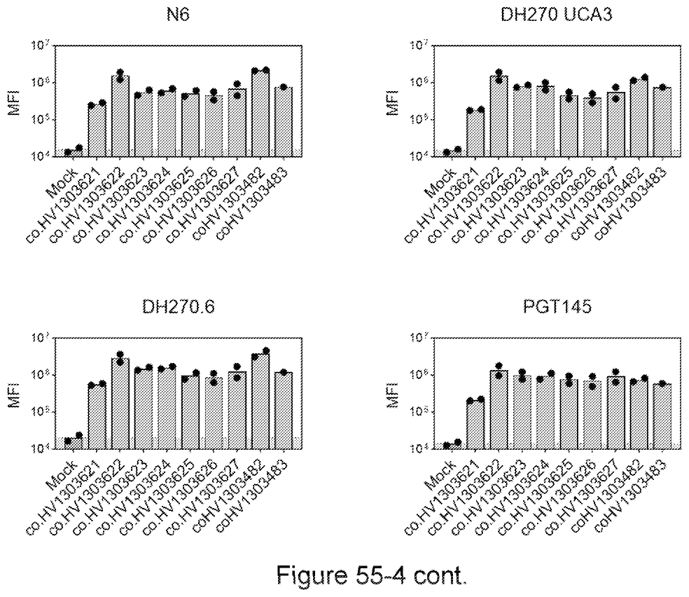

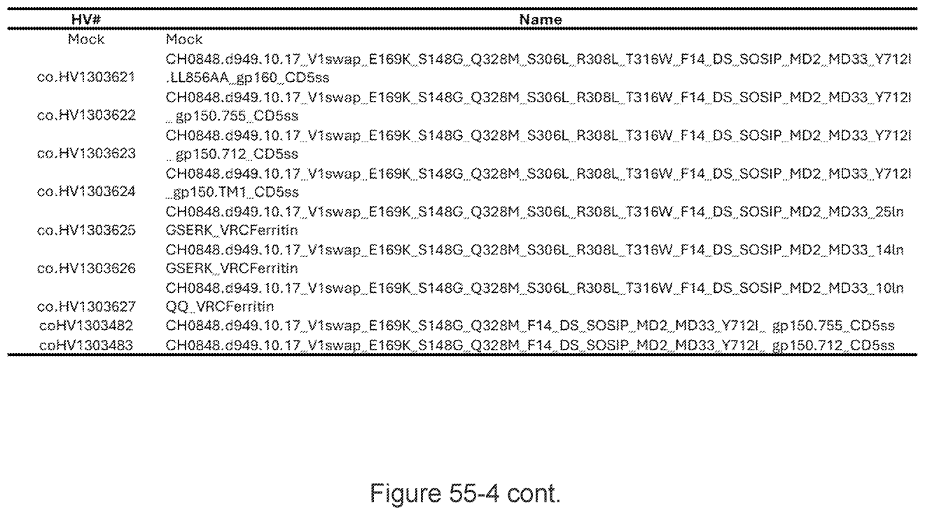

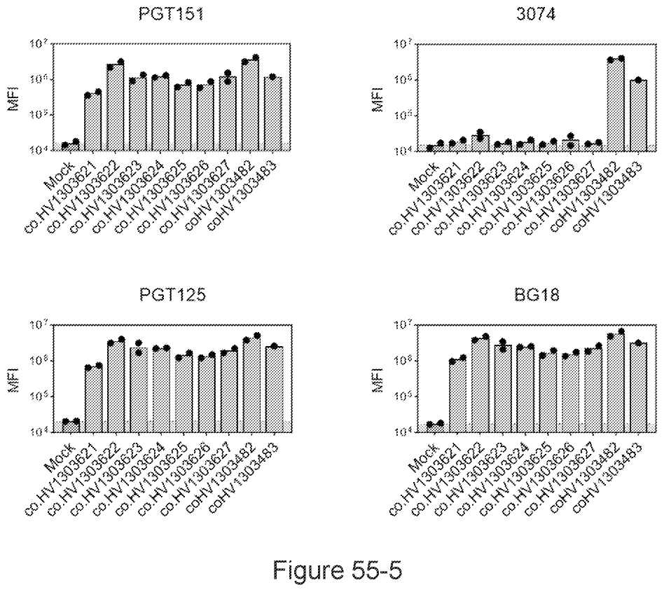

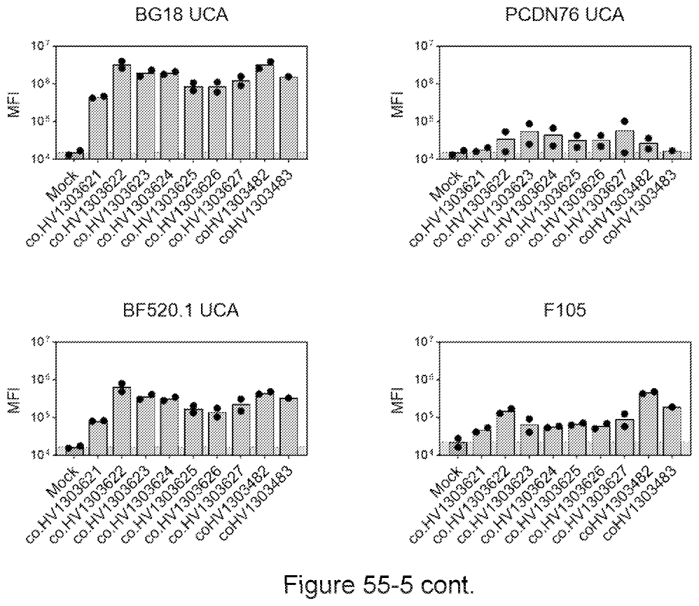

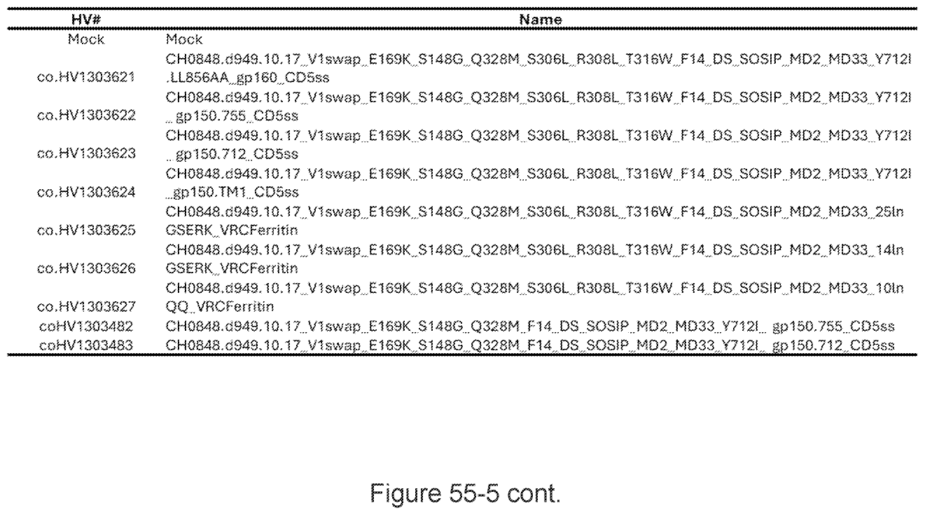

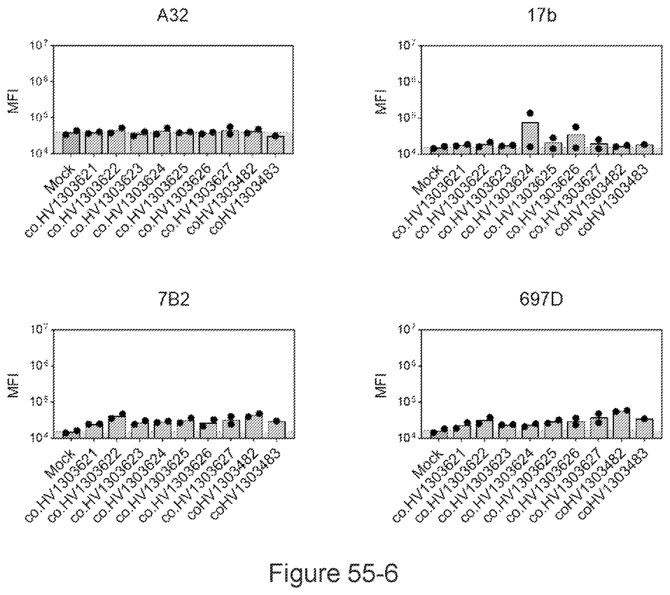

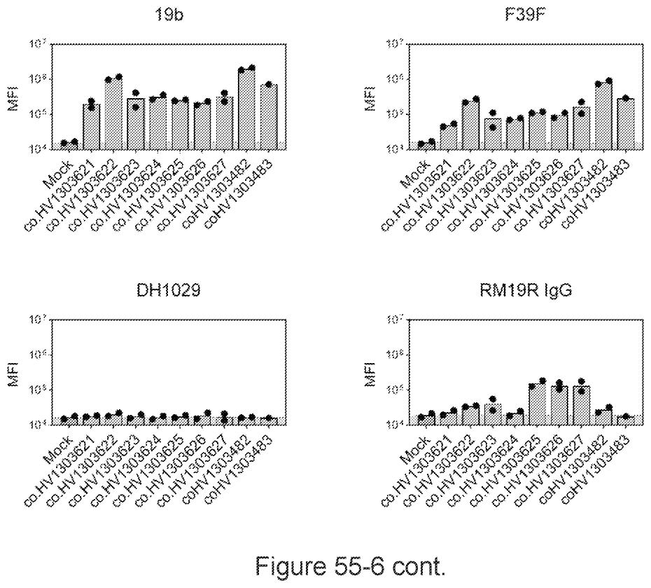

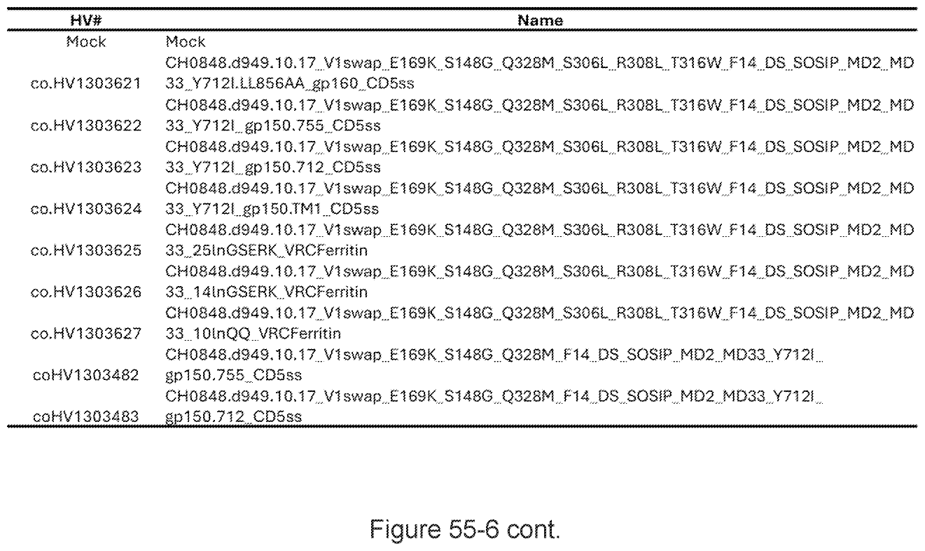

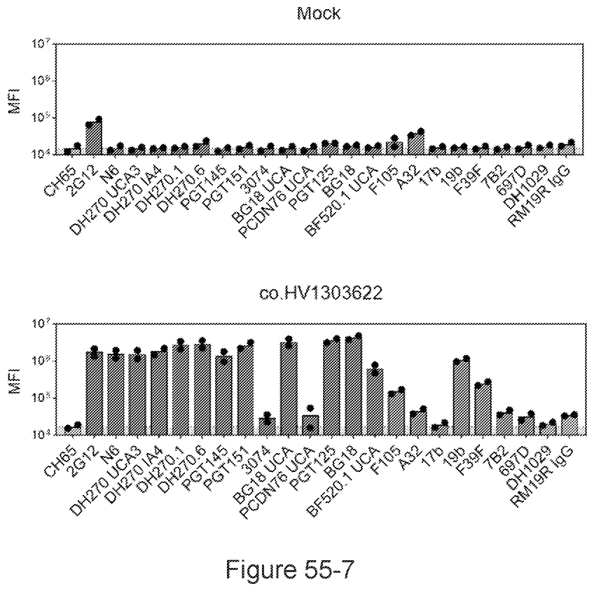

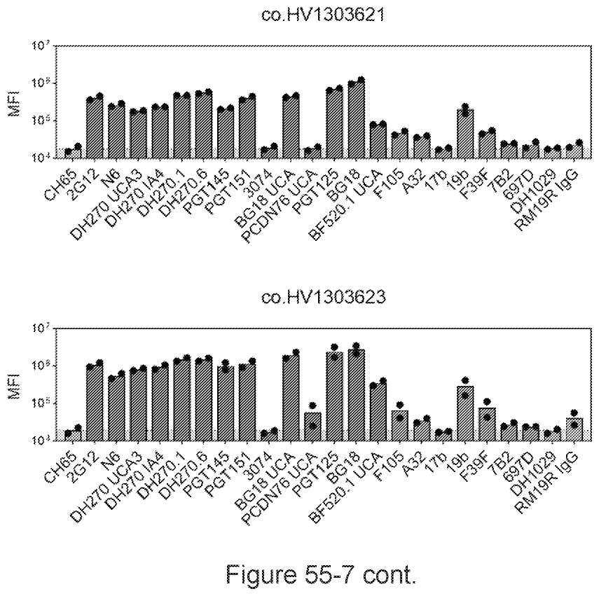

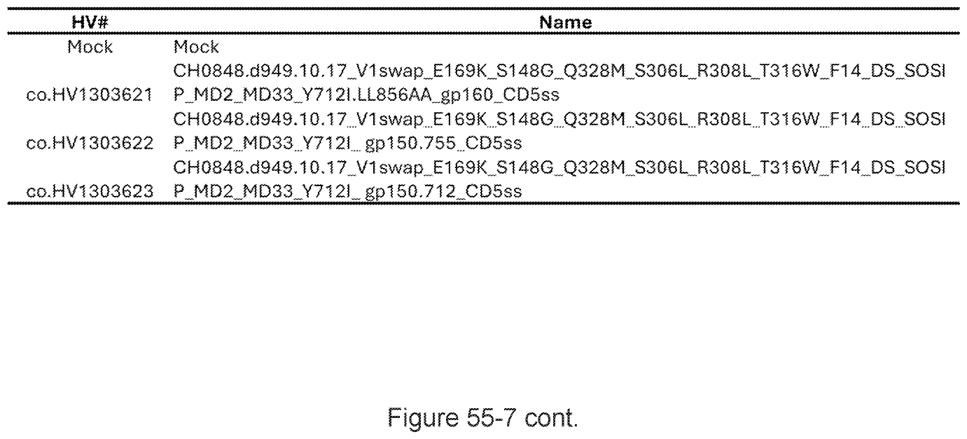

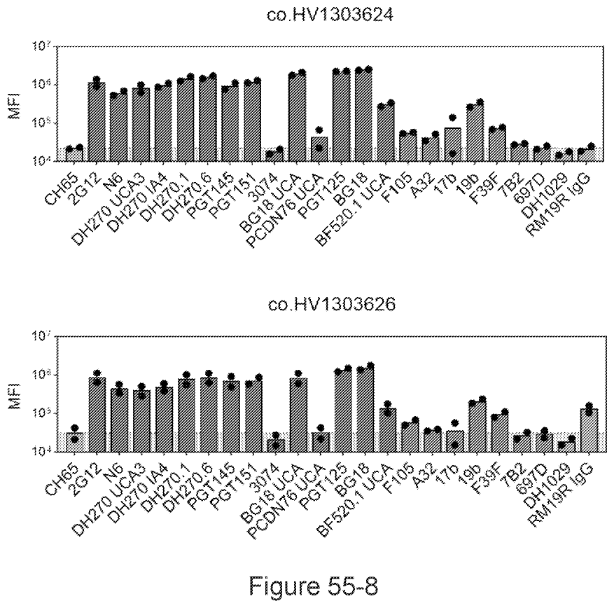

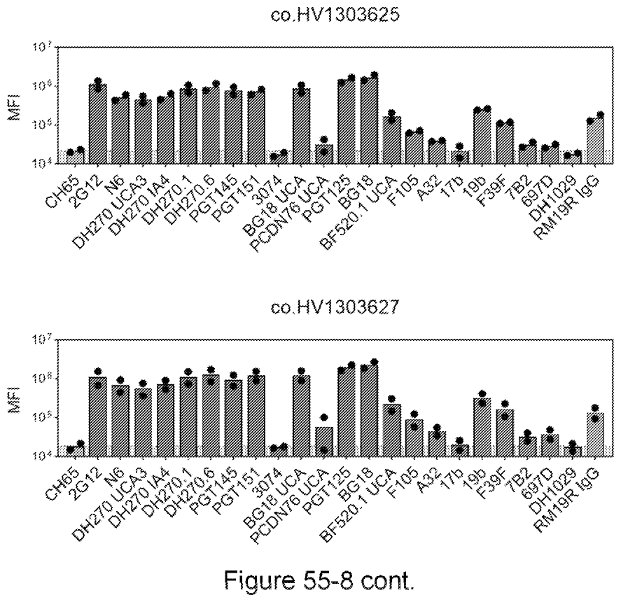

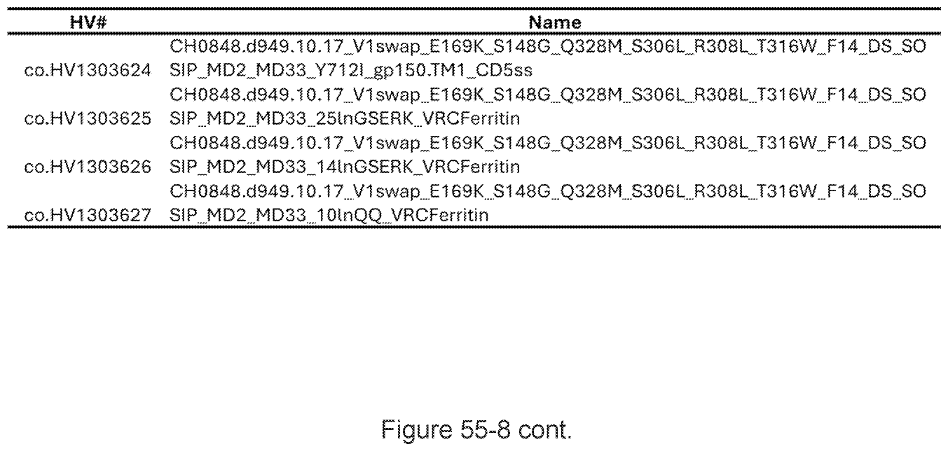

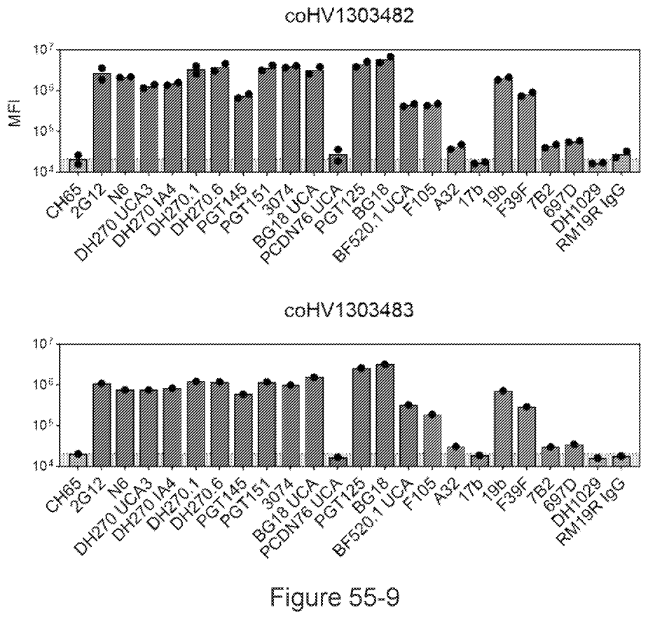

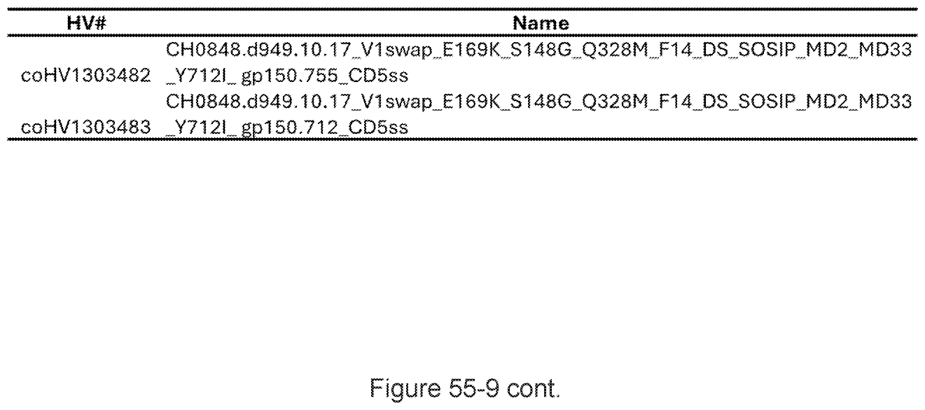

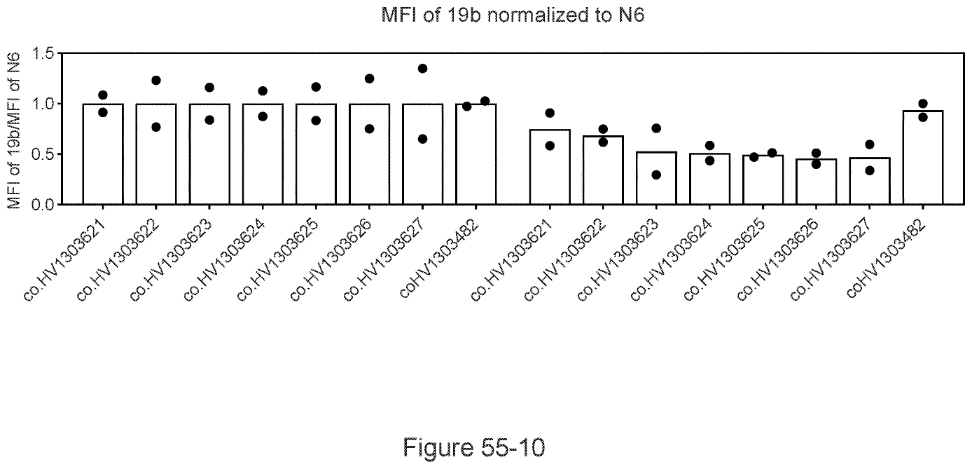

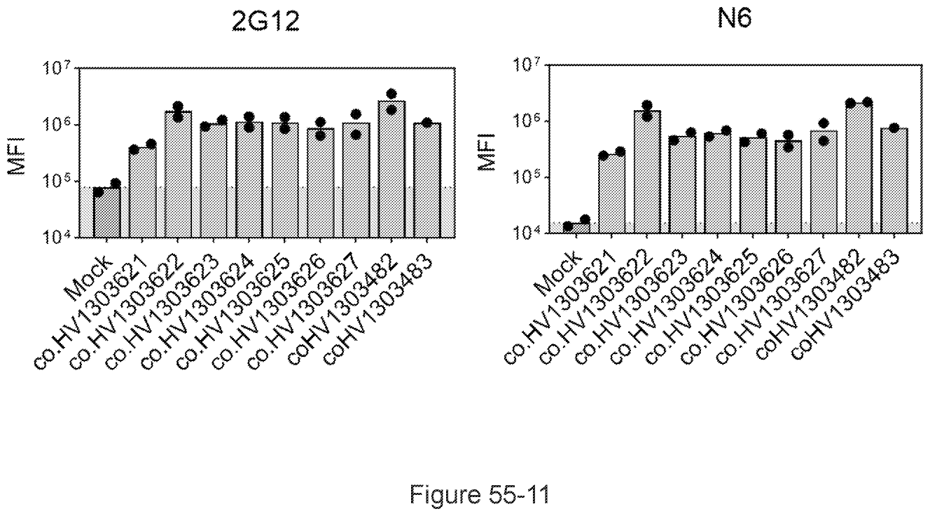

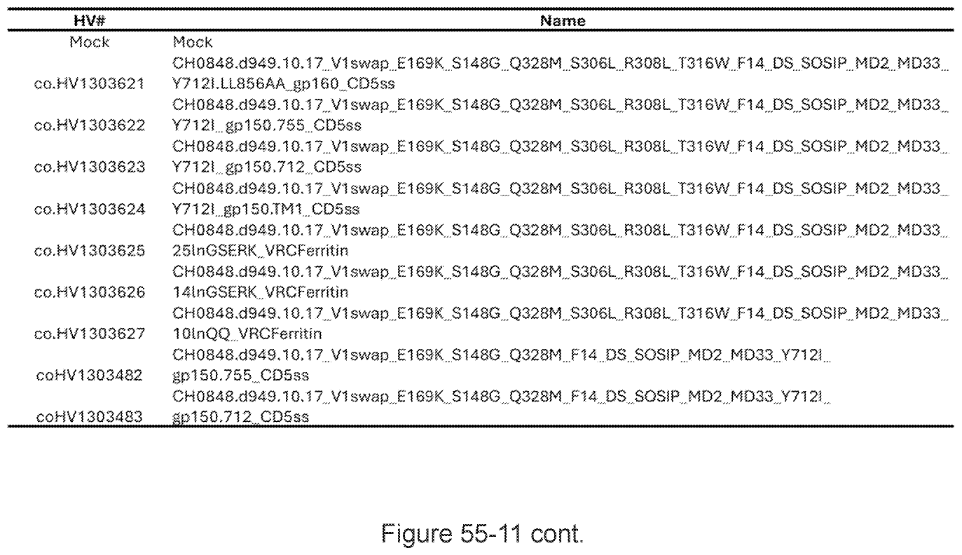

FIG. 55 parts 1-11 show V1swap envelops with V3 stabilizations (co.HV1303621-co.HV1303627, coHV1303482 and coHV1303483). coHV1303482 and coHV1303483 were best candidates from last round of designs. See FIG. 55-1. FIG. 55-2 shows a list of tested antibodies. FIG. 55-3 shows the heatmap of MFI of all antibodies binding to all Envs expressed from mRNA constructs. FIGS. 55-4-55-9 show the binding reactions of Envs co.HV1303621-co.HV1303627, coHV1303482 and coHV1303483. FIG. 55-11 shows 19b binding normalized to N6. FIG. 55-9 shows how tail truncation affected expression levels. 2G12 and N6 binding was used as a surrogate as expression levels (gp150.755>gp150.712 (=gp150.TM1)>gp160).

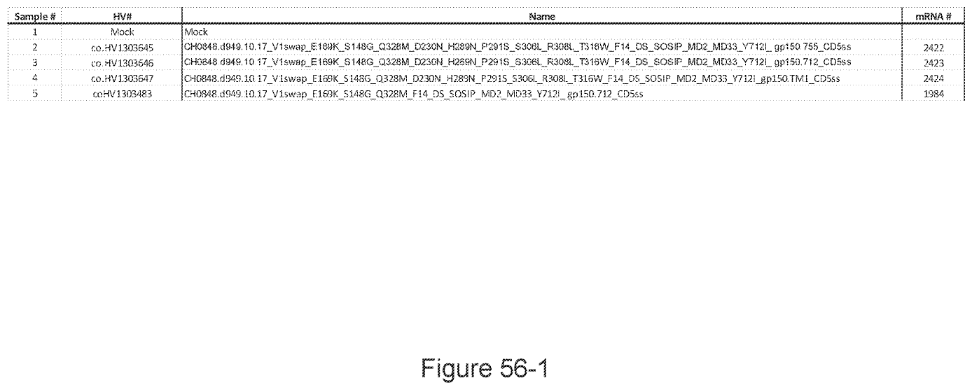

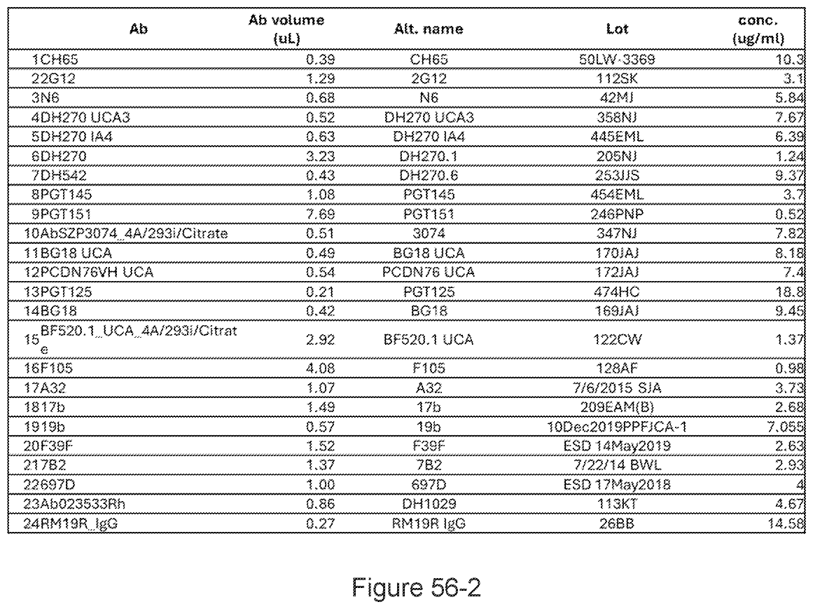

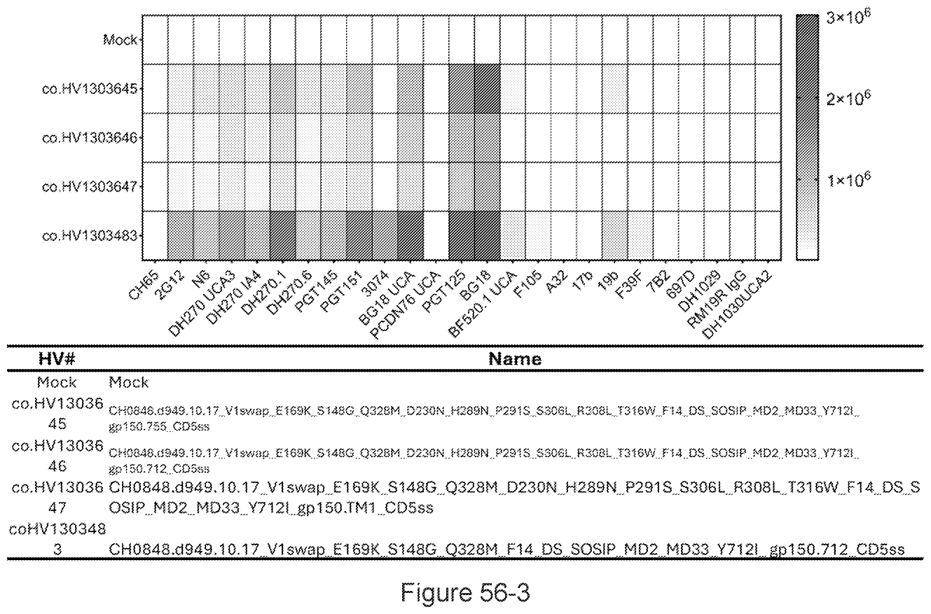

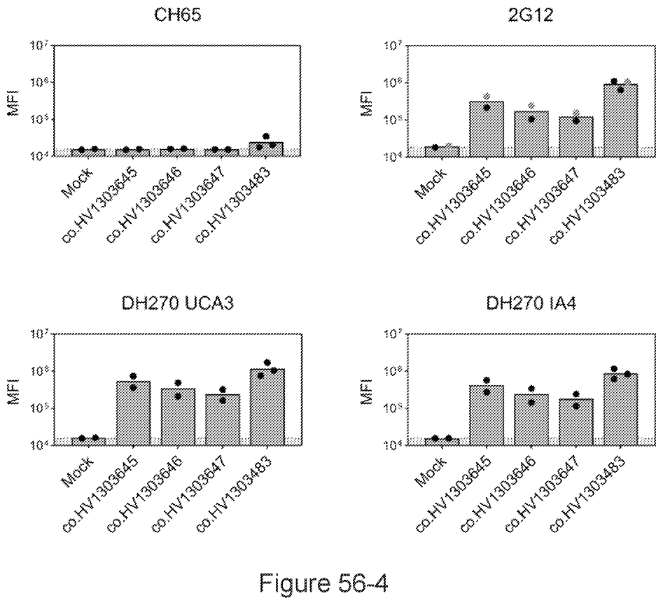

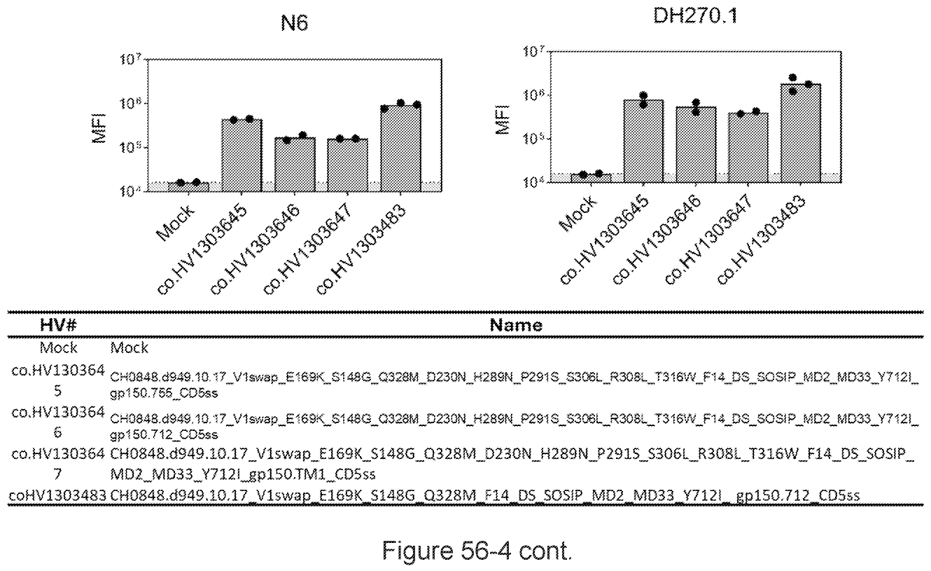

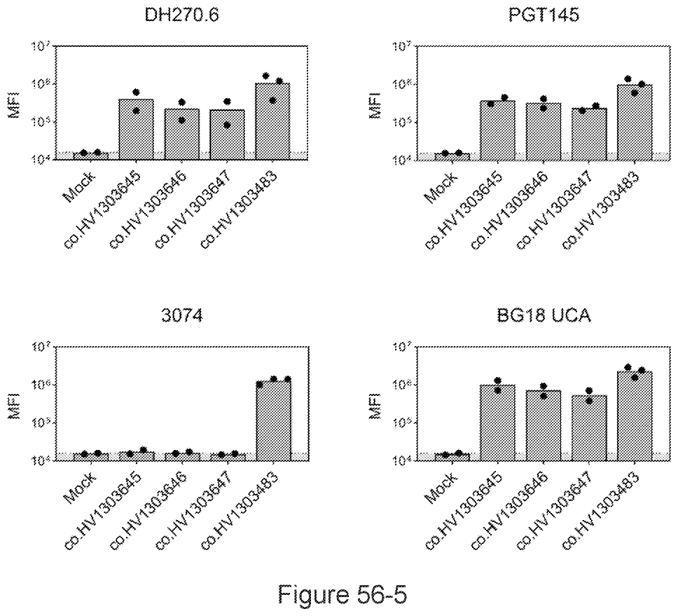

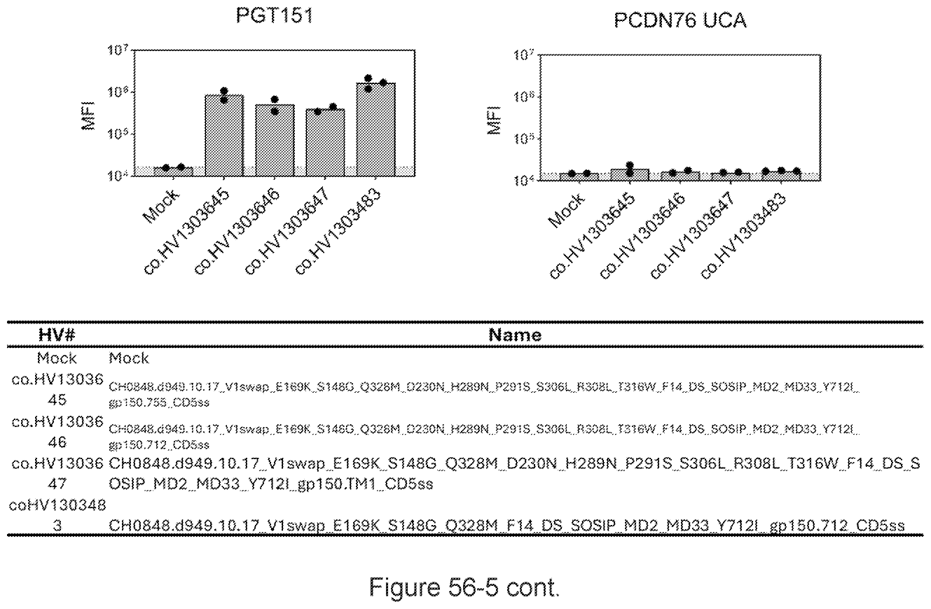

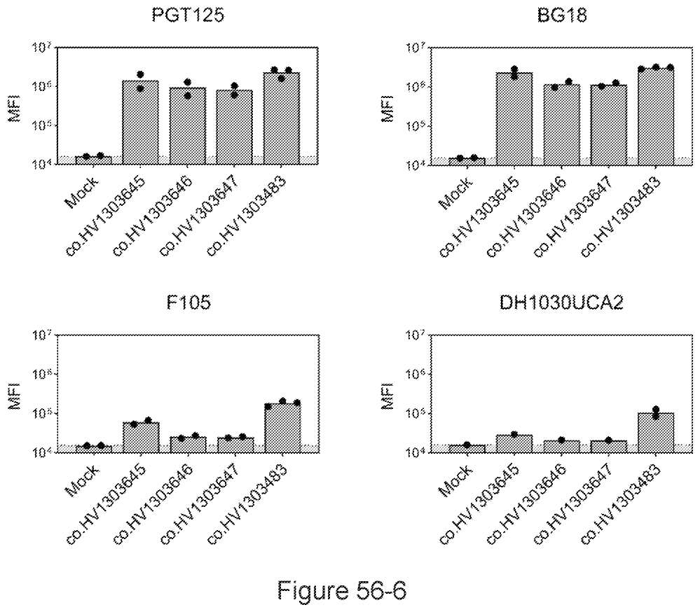

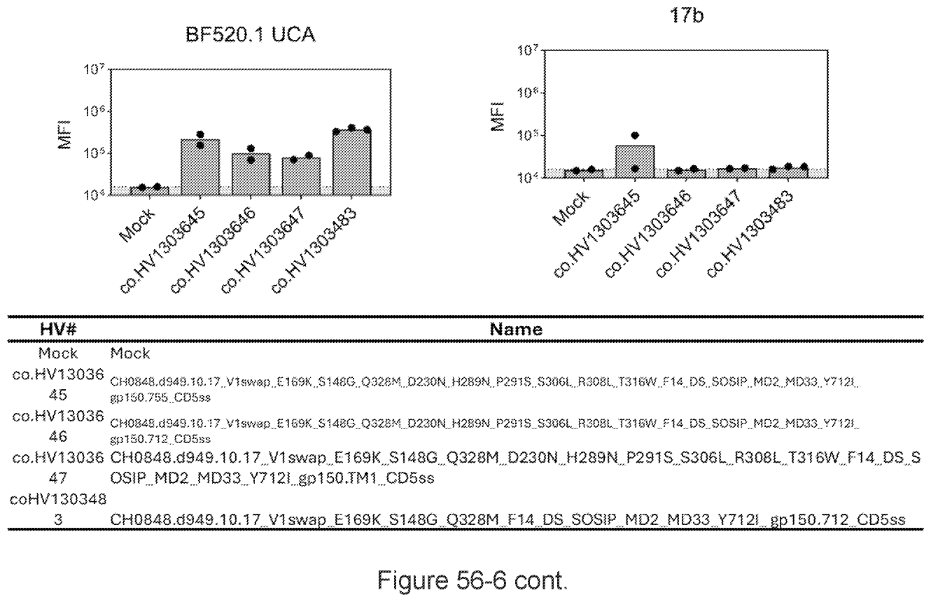

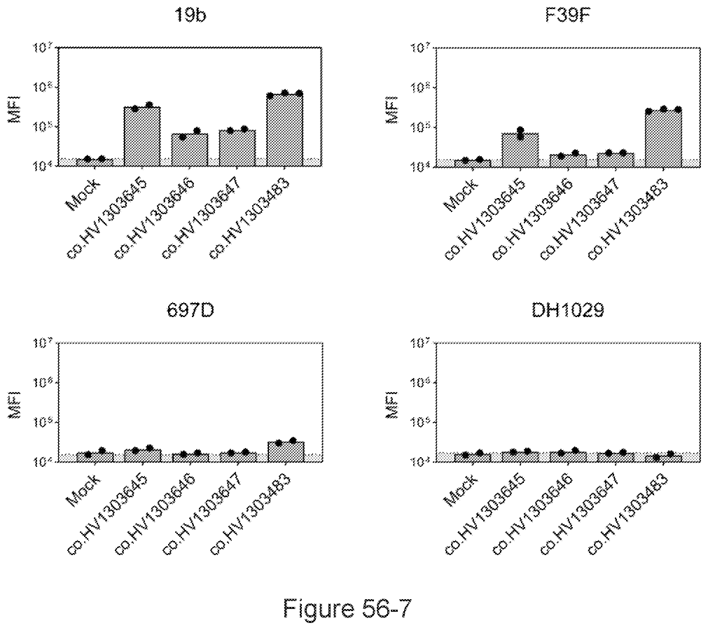

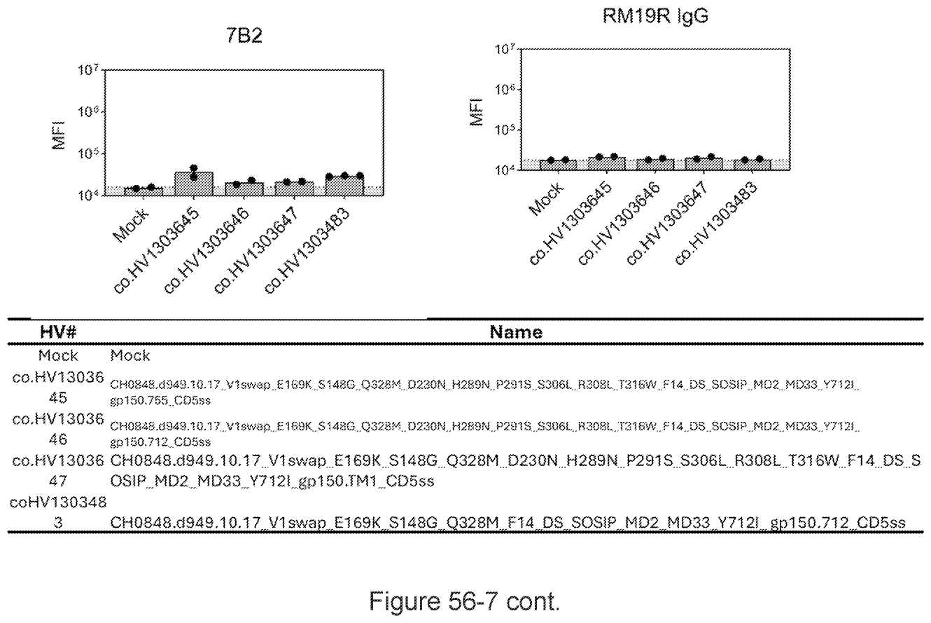

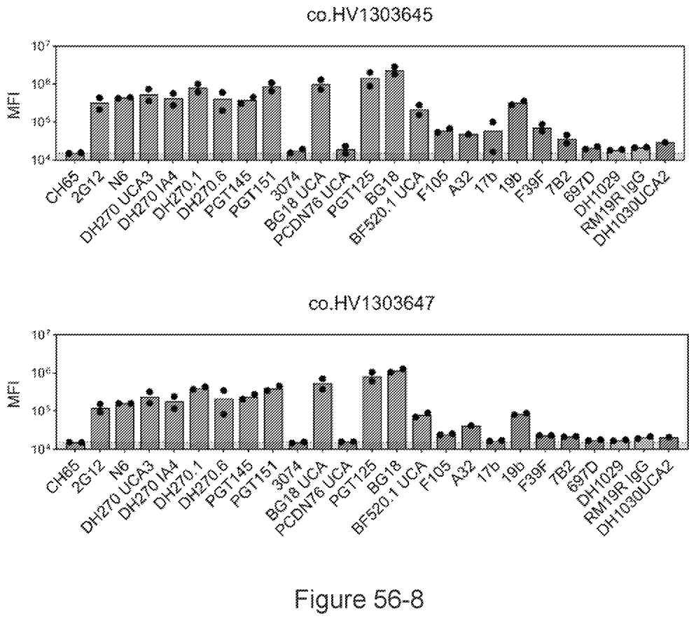

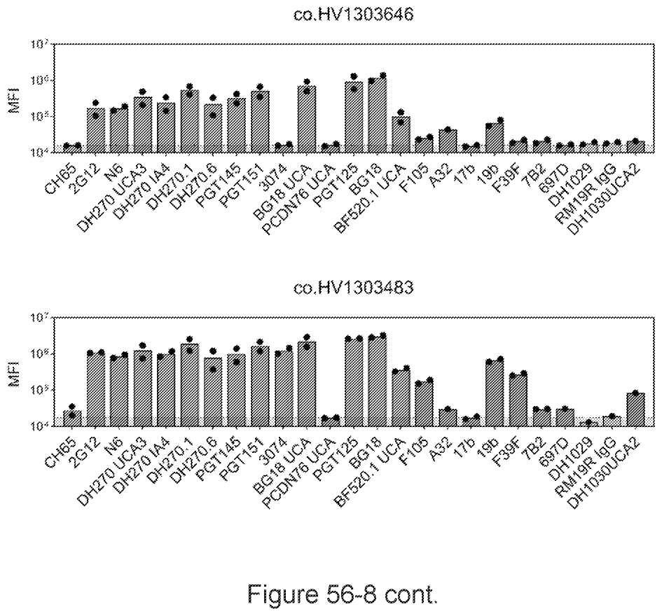

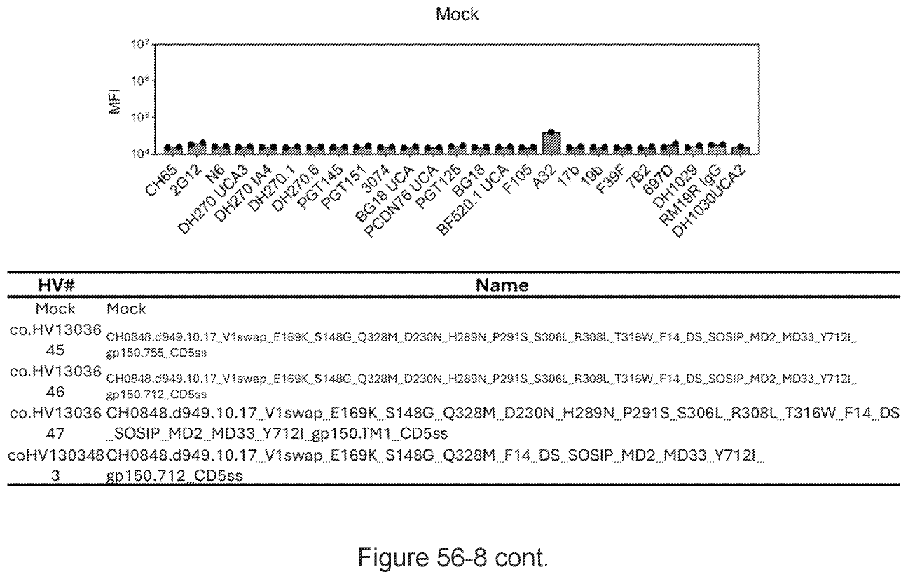

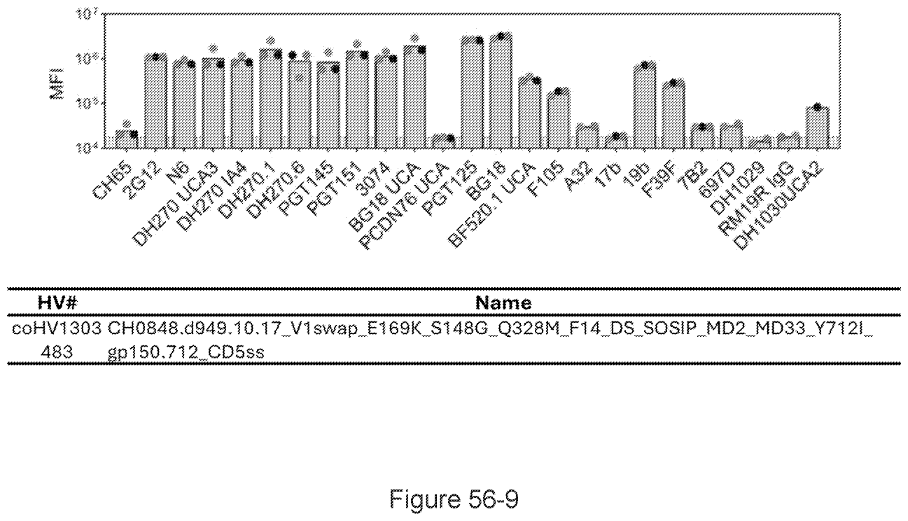

FIG. 56 parts 1-9 show MB Env v1swap envelopes (co.HV1303645-co.HV1303647 and coHV1303483). FIG. 56-2 shows a list of tested antibodies. FIG. 56-3 shows the heatmap of MFI of all antibodies binding to all Envs expressed from mRNA constructs. FIGS. 56-4-56-8 show the binding reactions of Envs co.HV1303645-co.HV1303647 and coHV1303483. FIG. 56-9 shows the binding reactions of Env coHIV1303483.

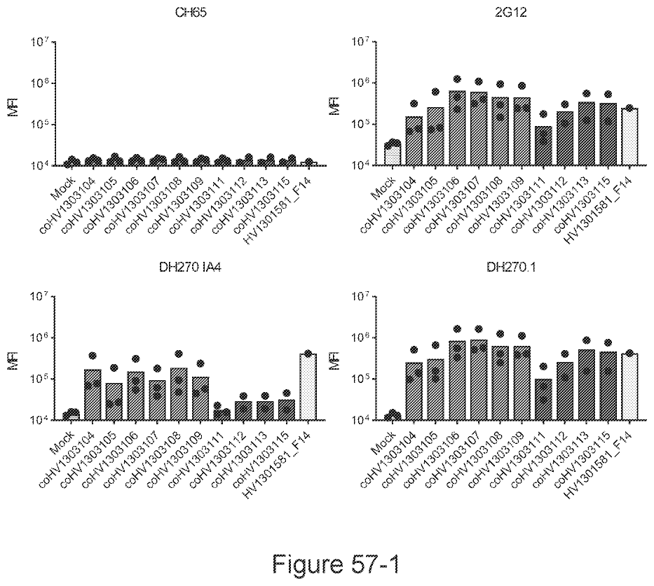

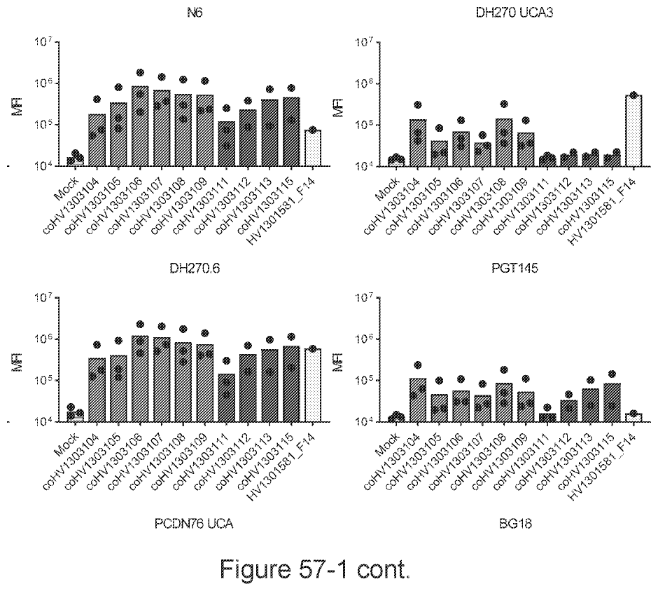

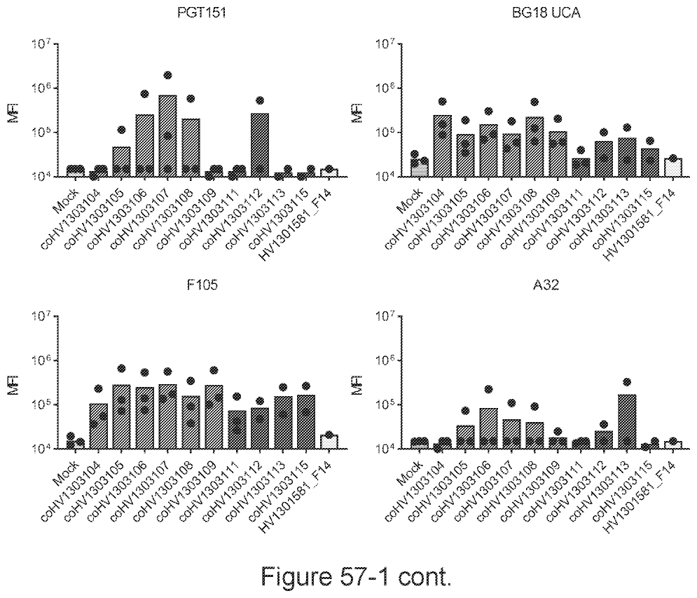

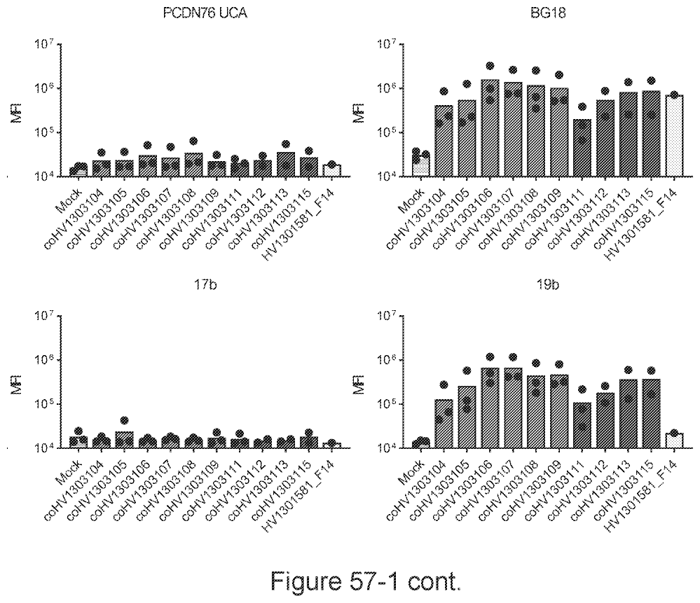

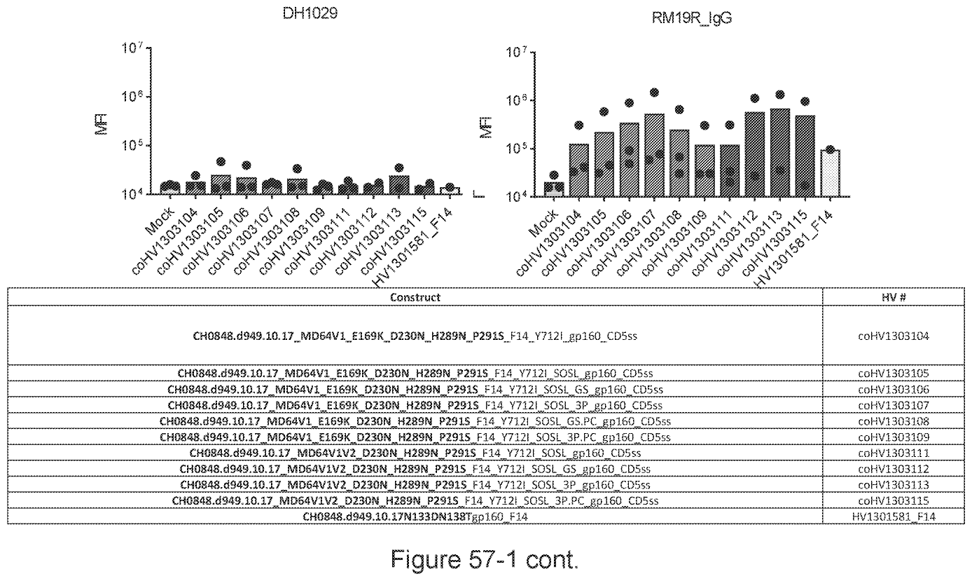

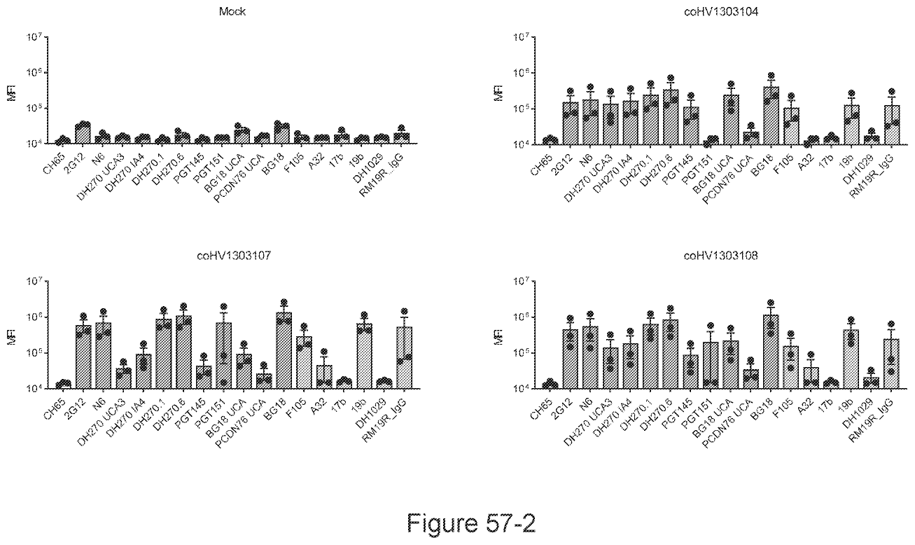

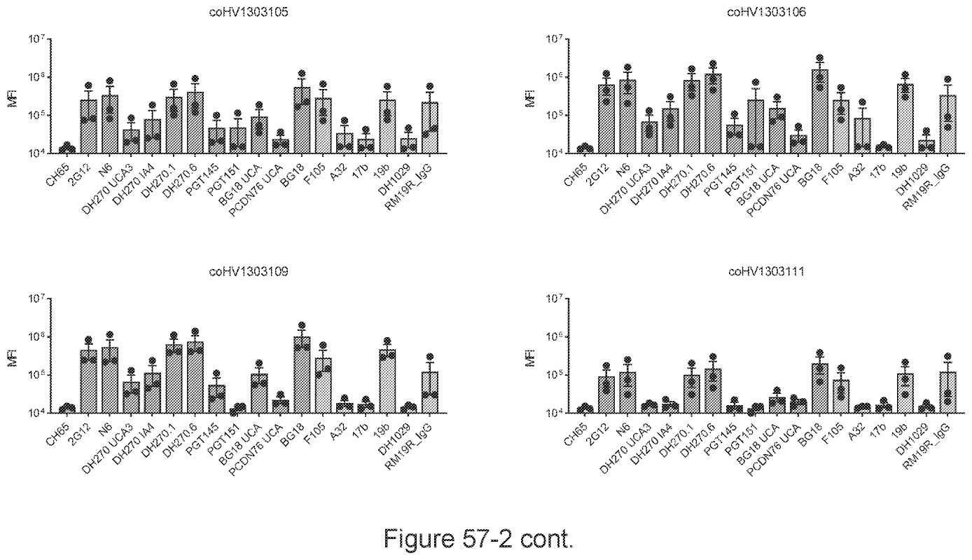

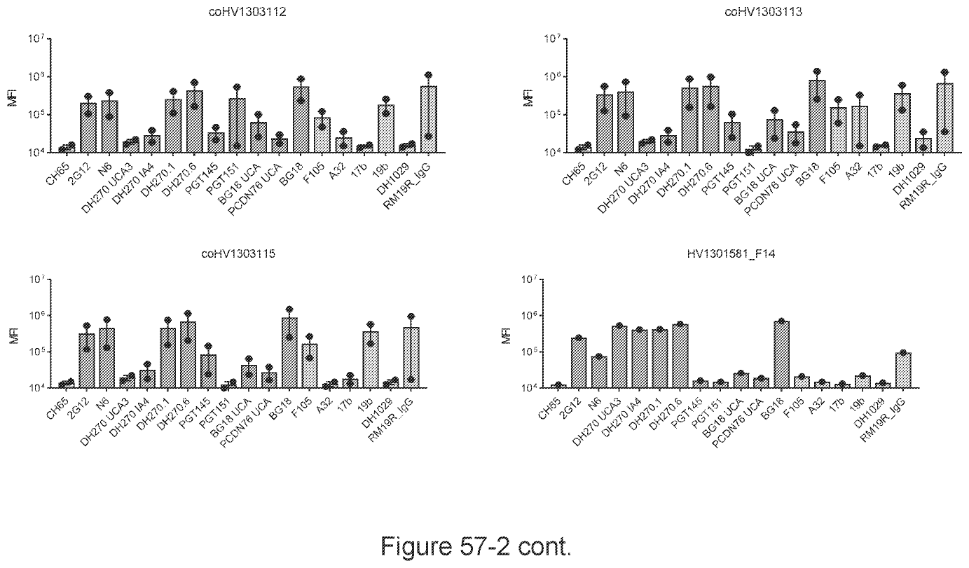

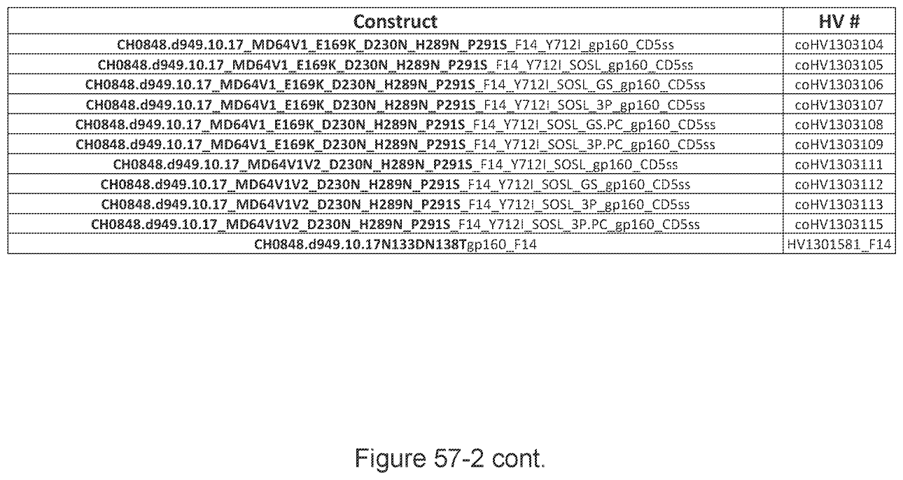

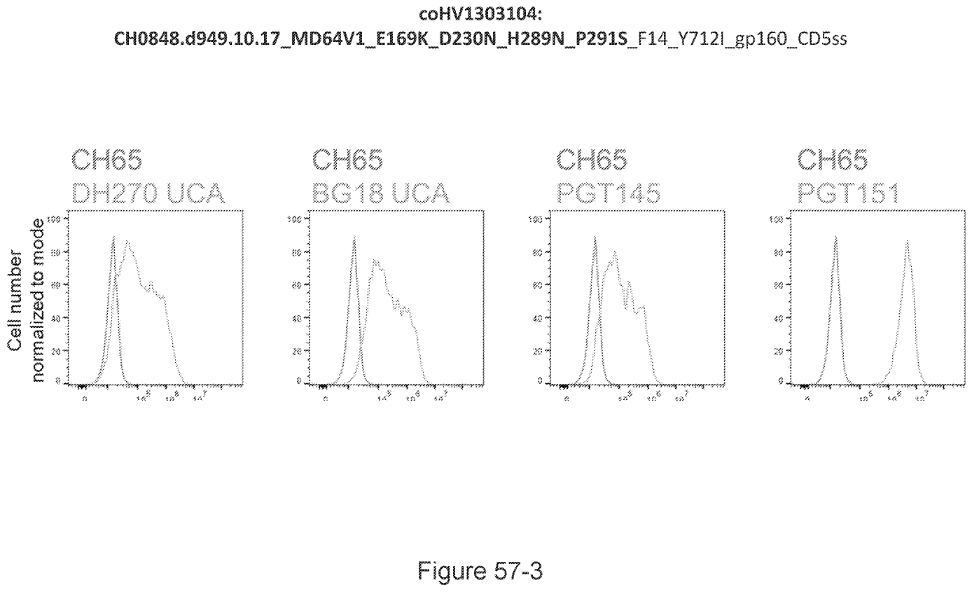

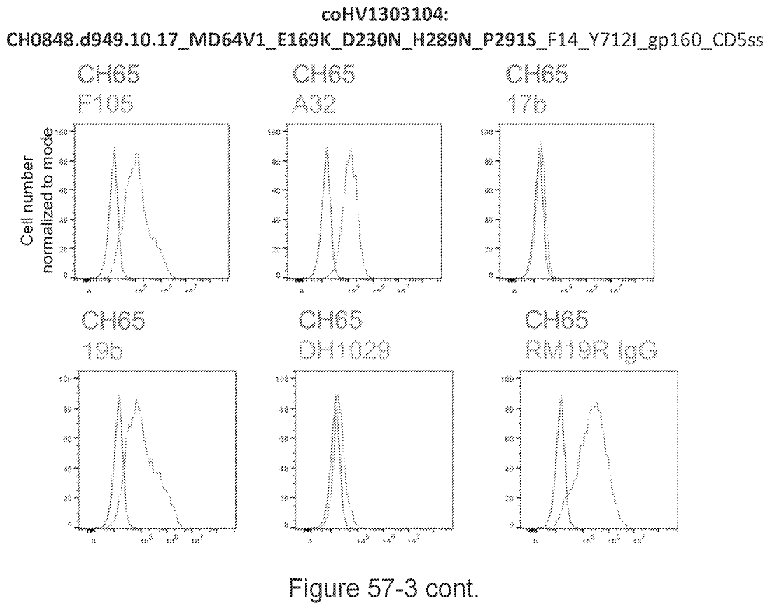

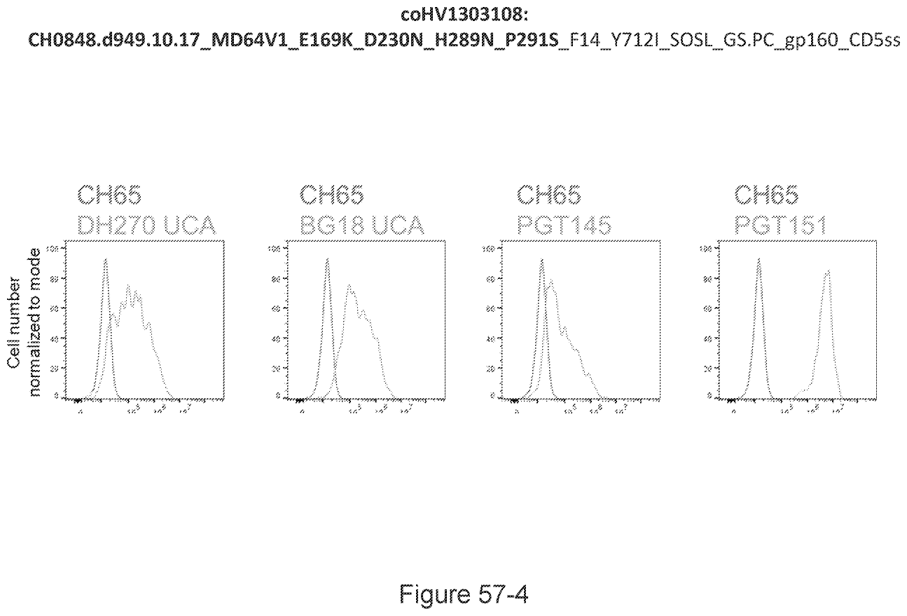

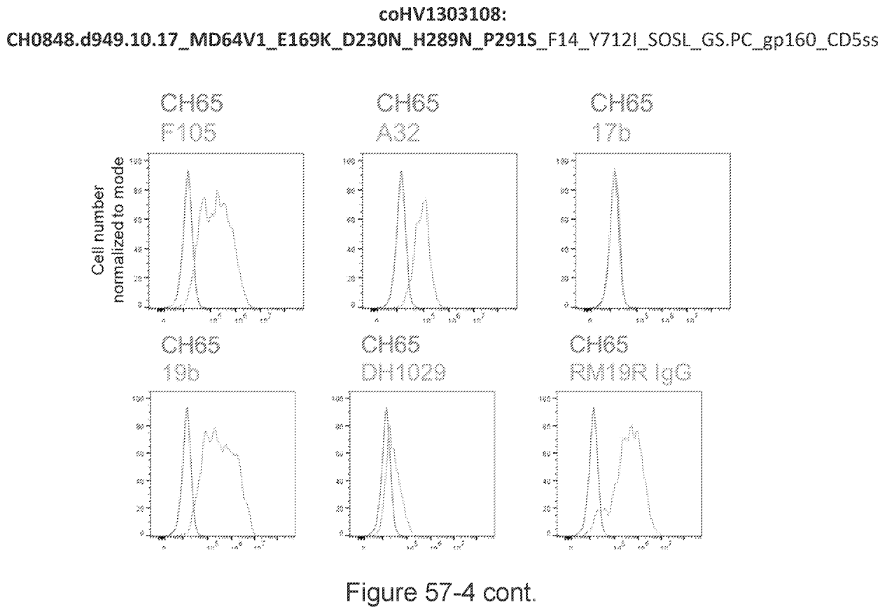

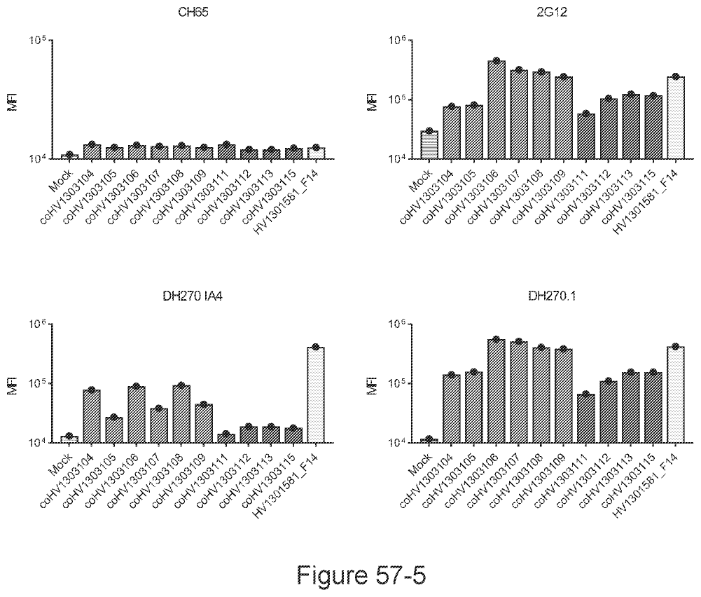

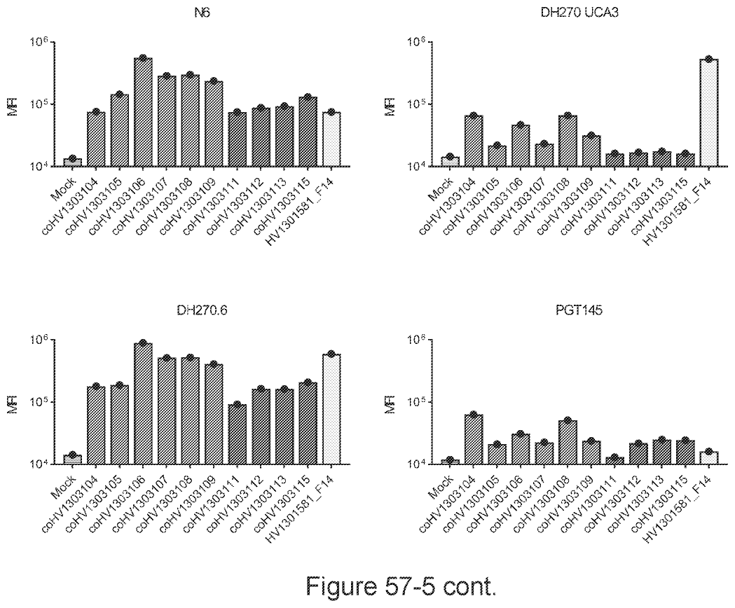

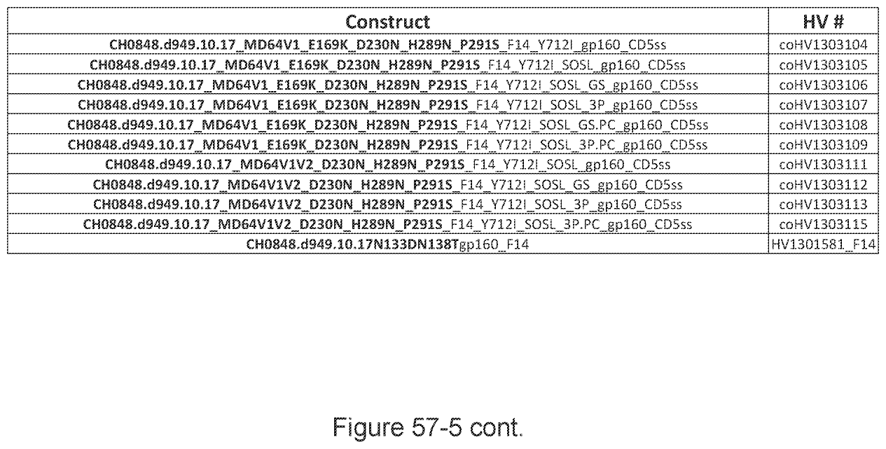

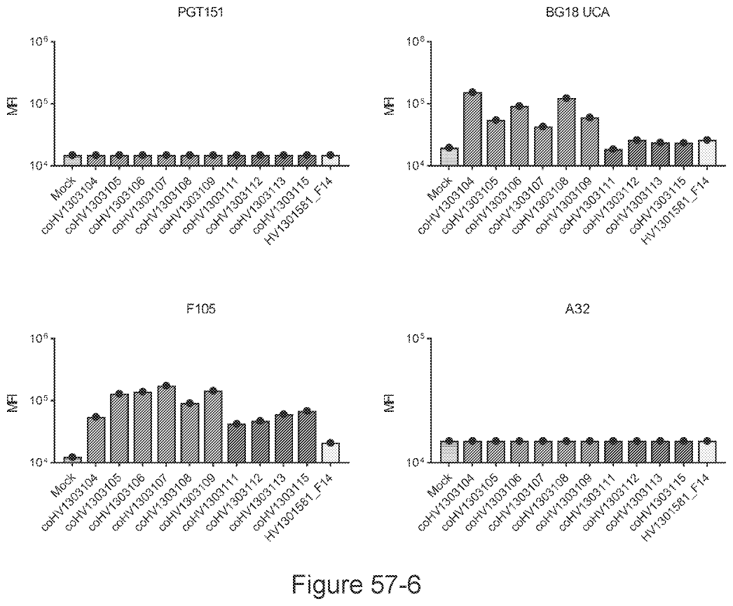

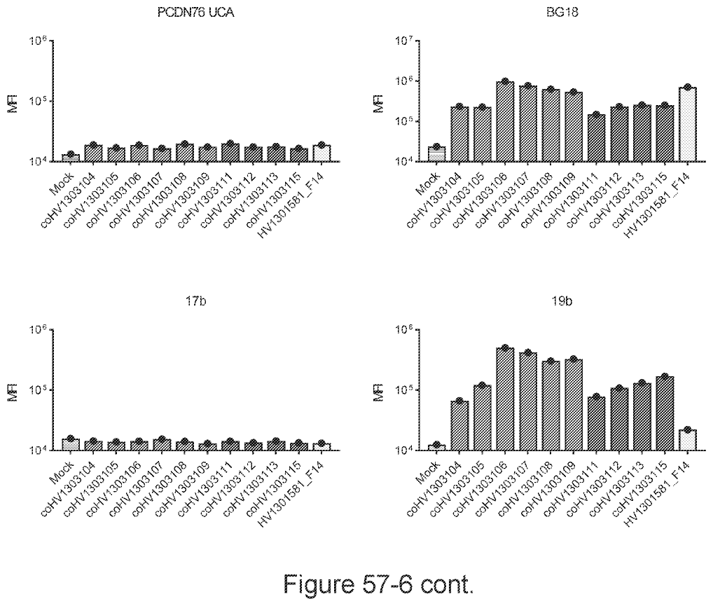

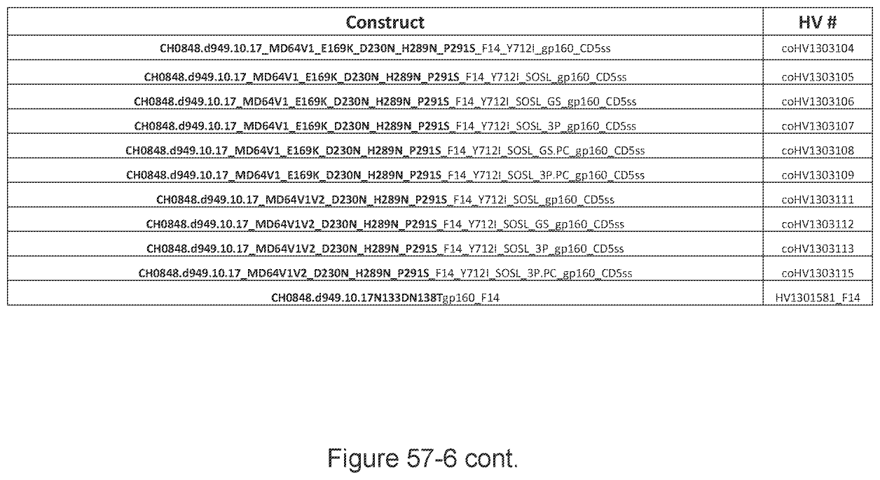

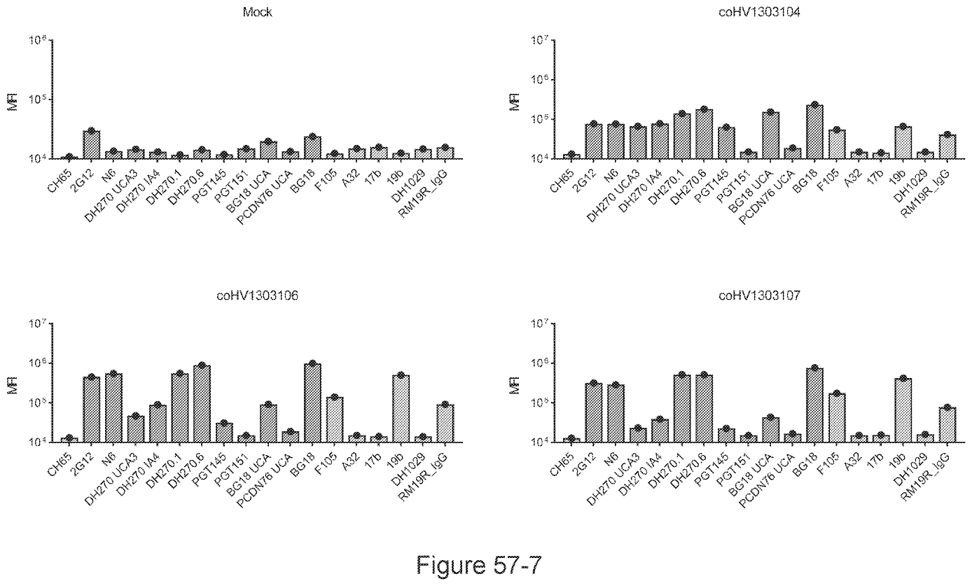

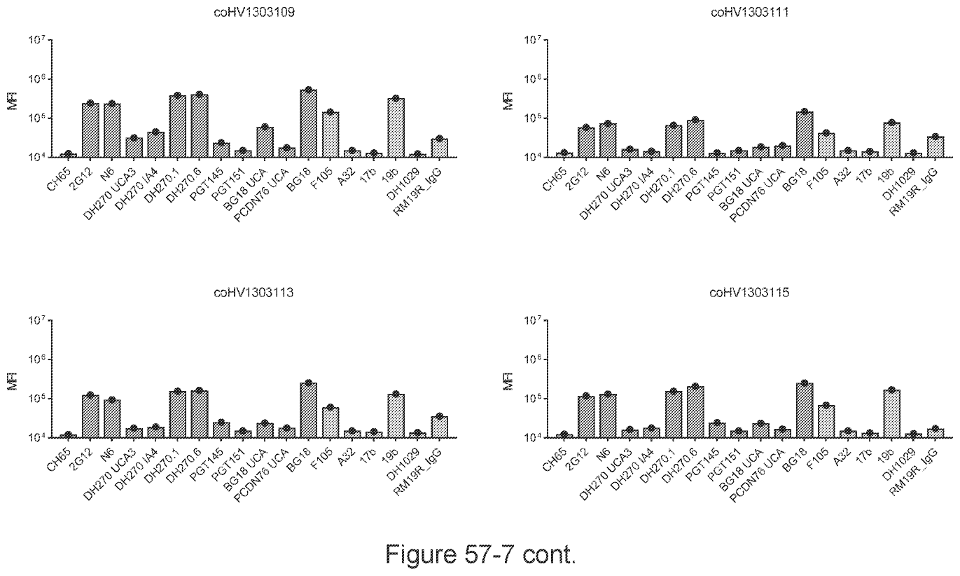

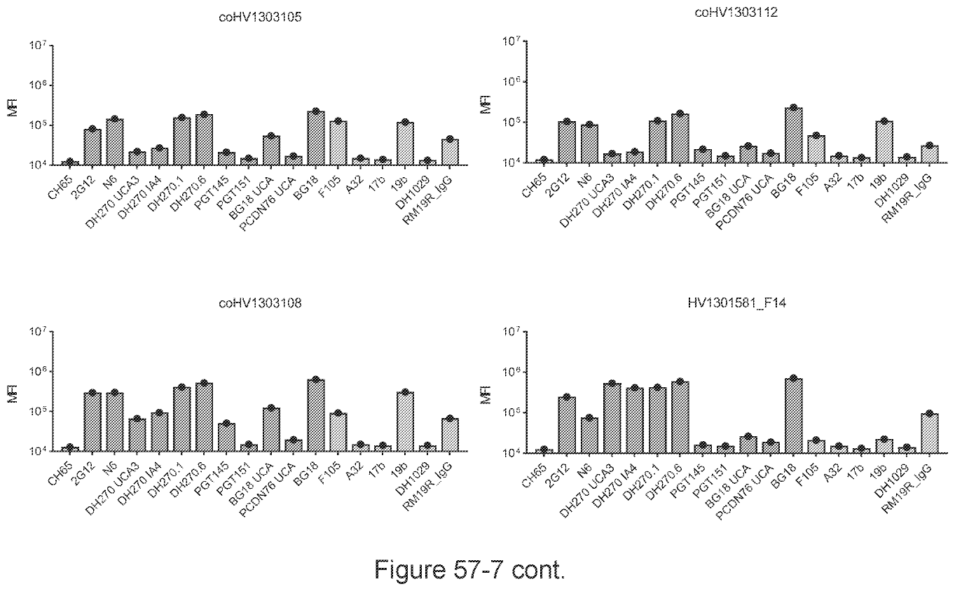

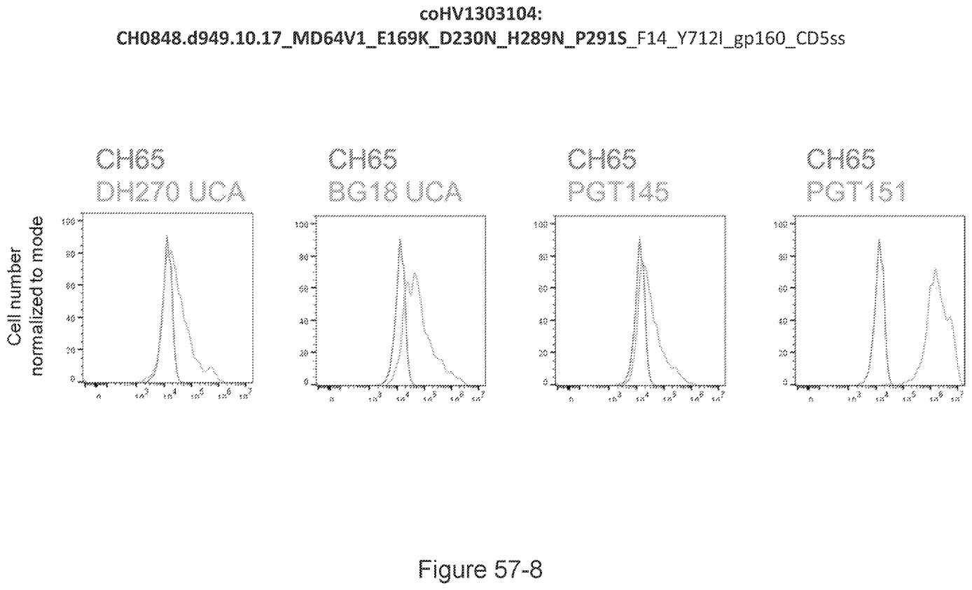

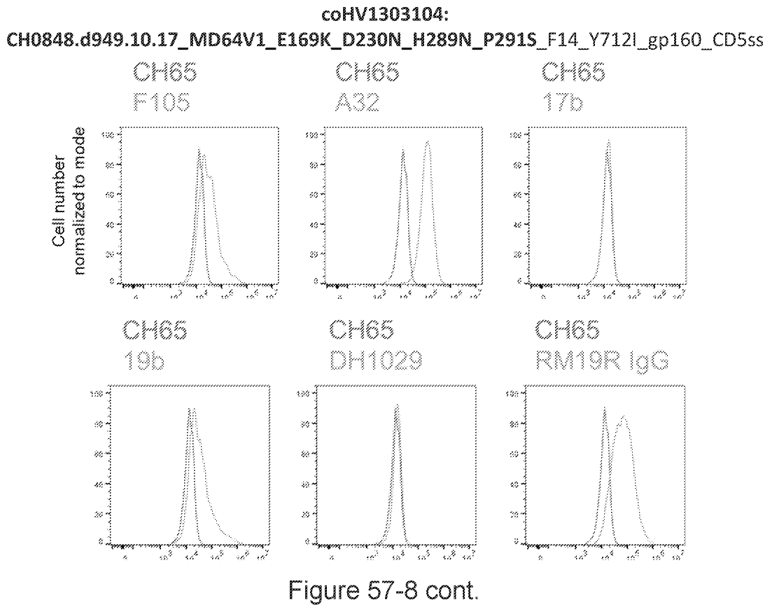

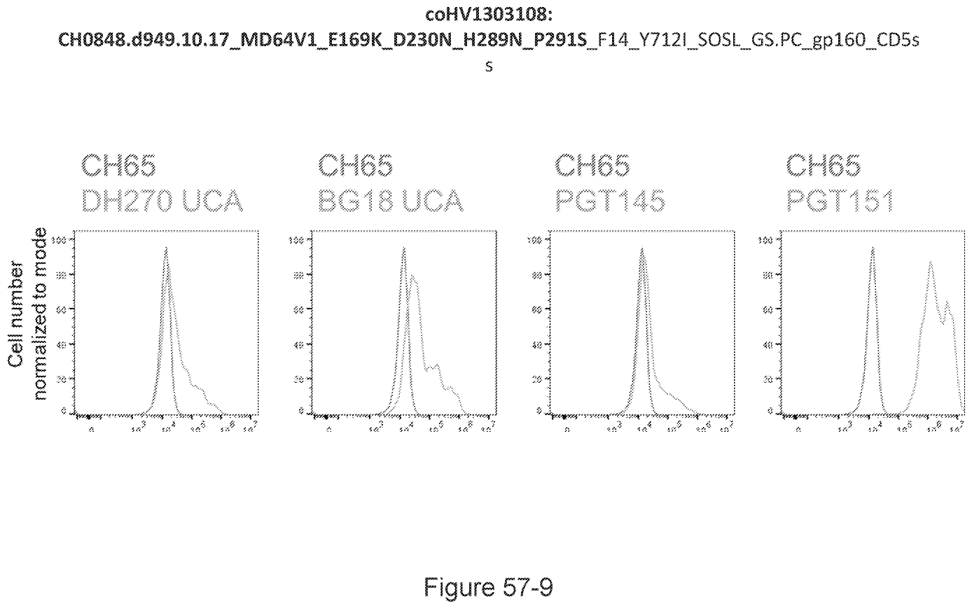

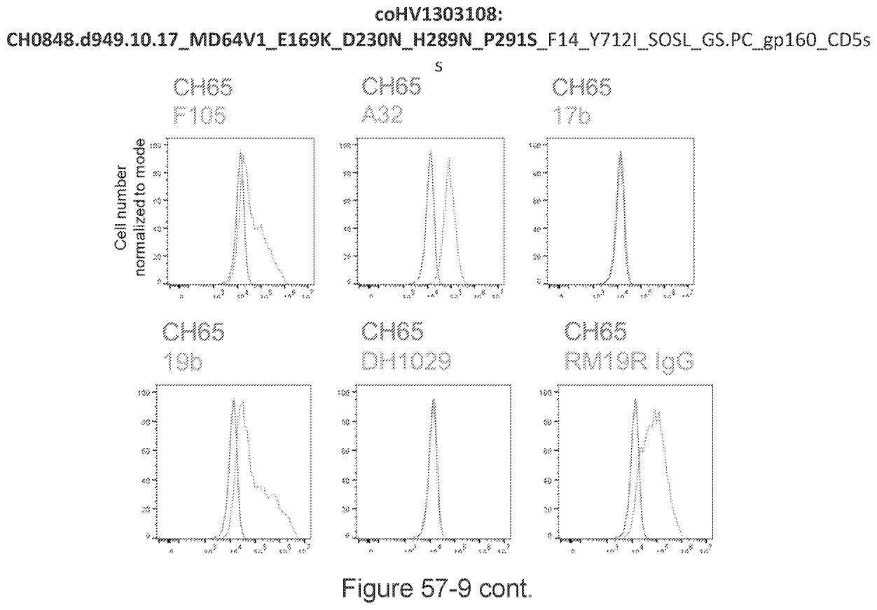

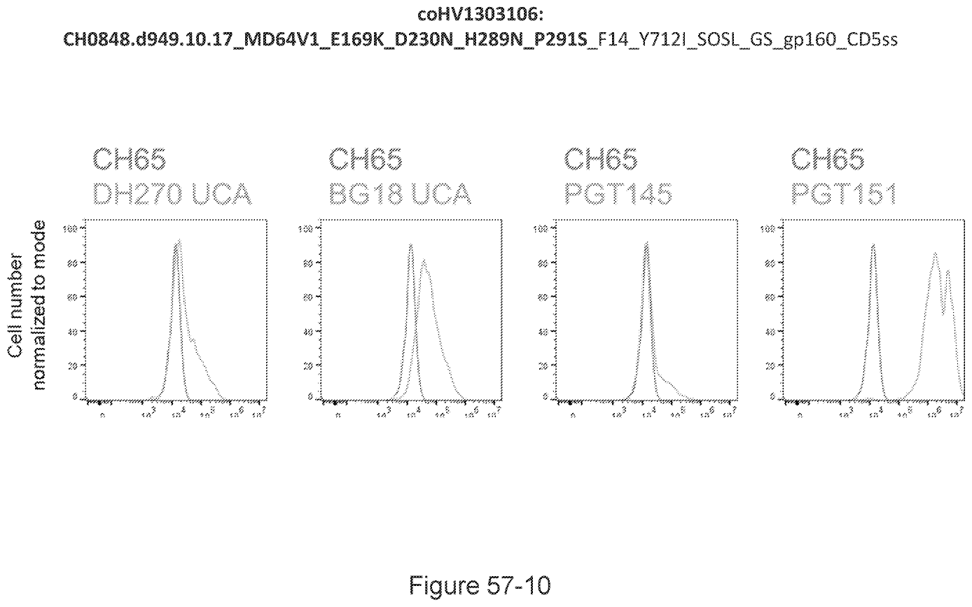

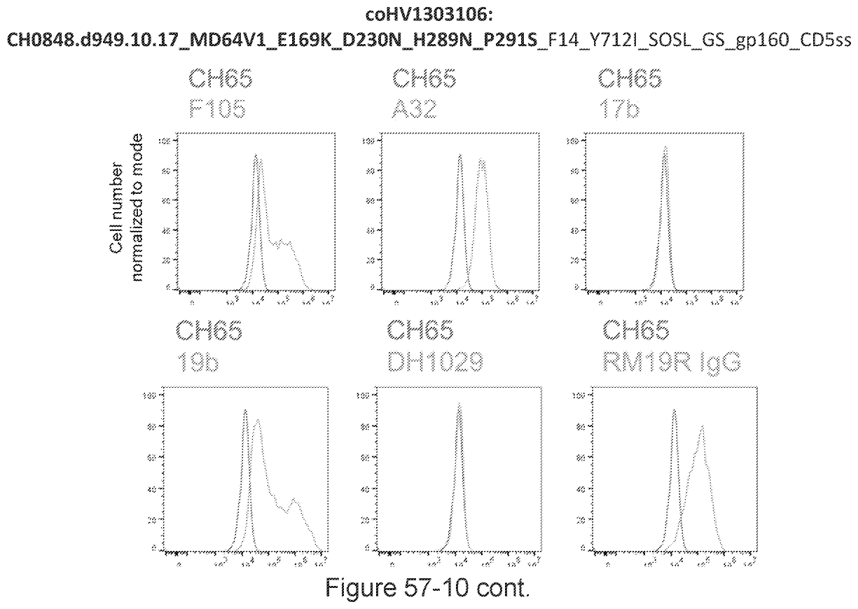

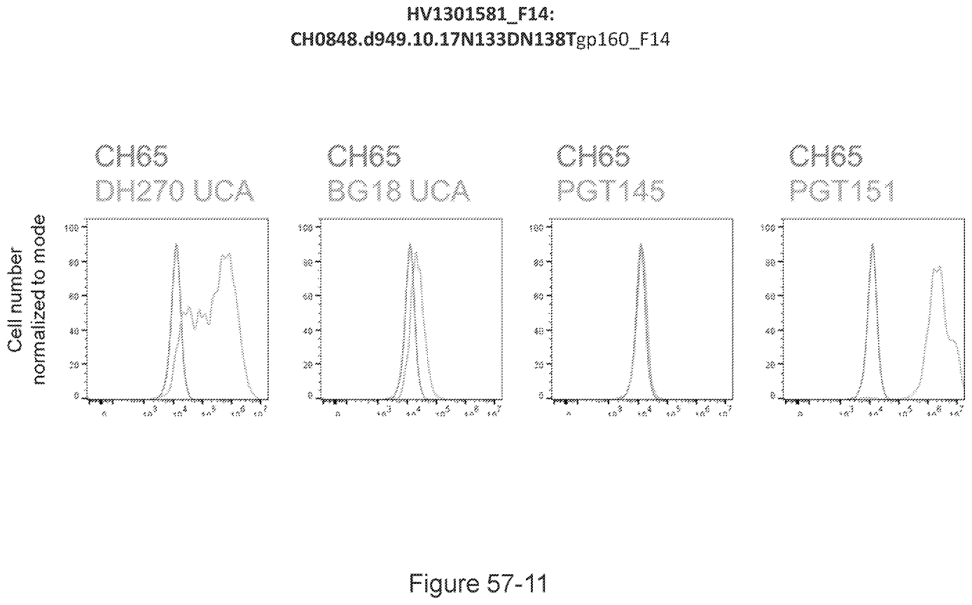

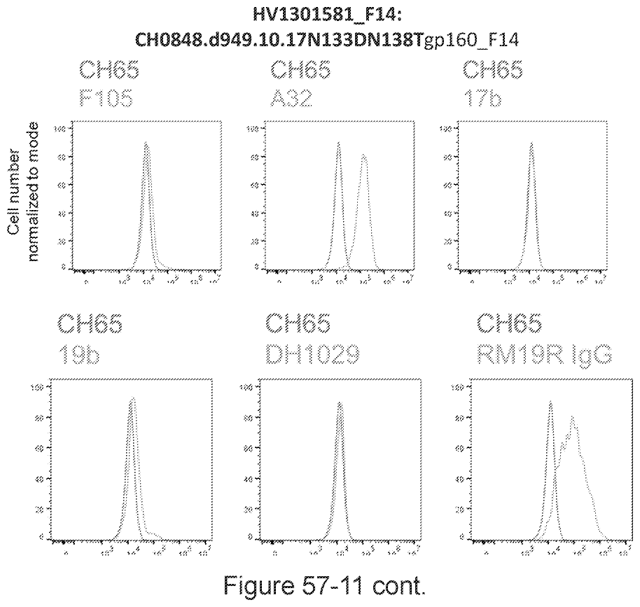

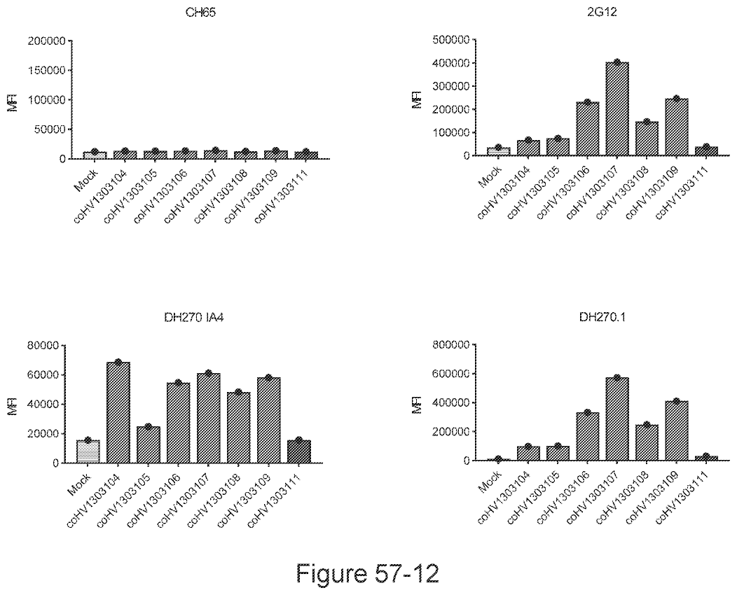

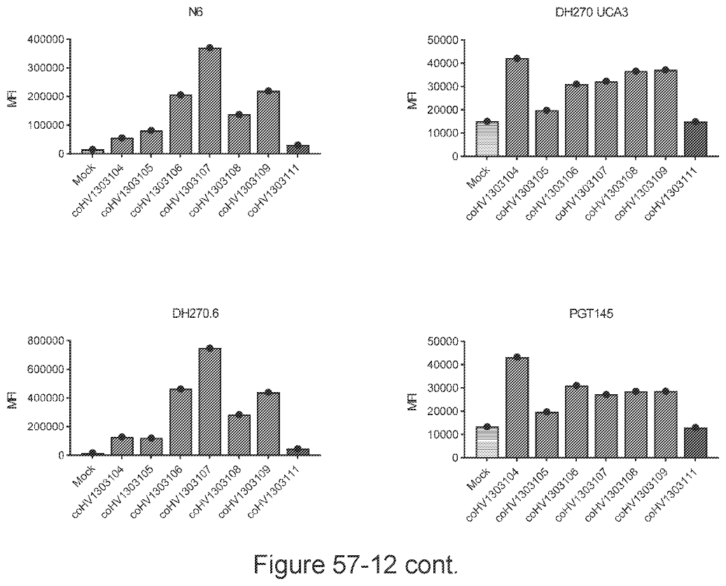

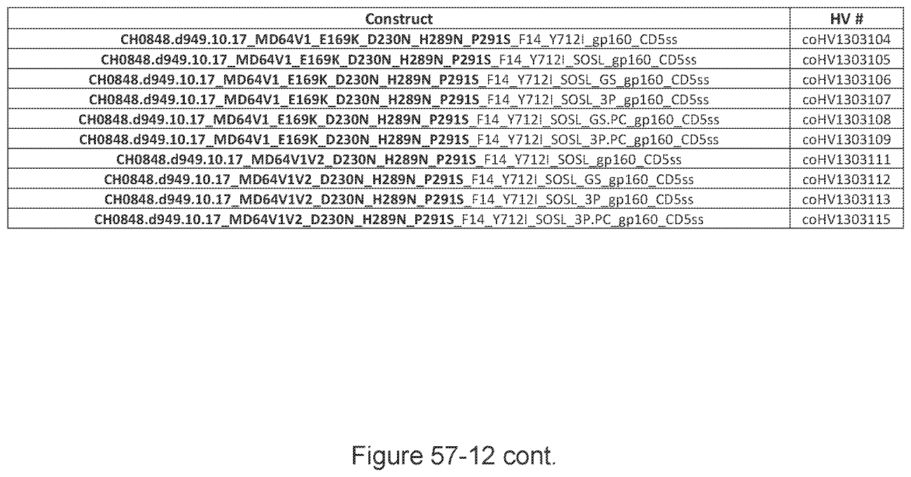

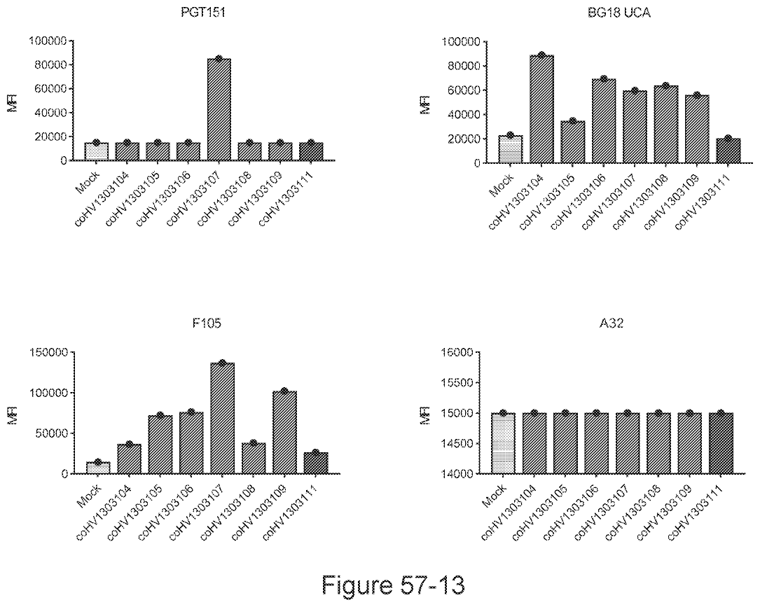

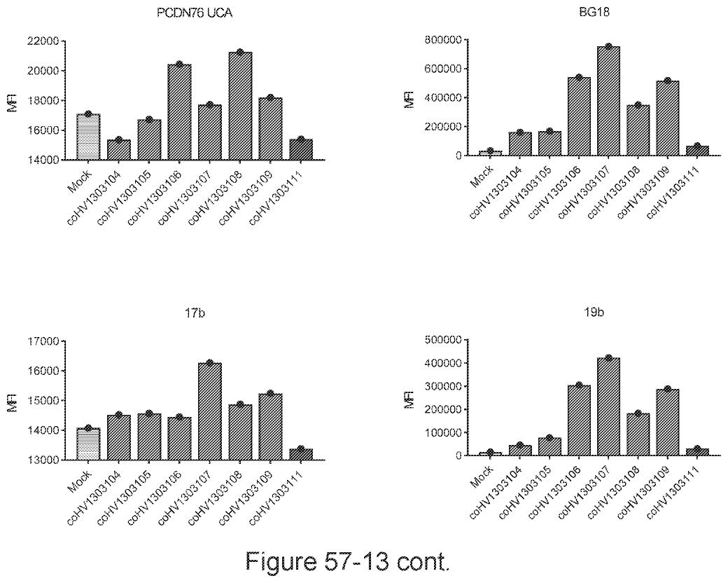

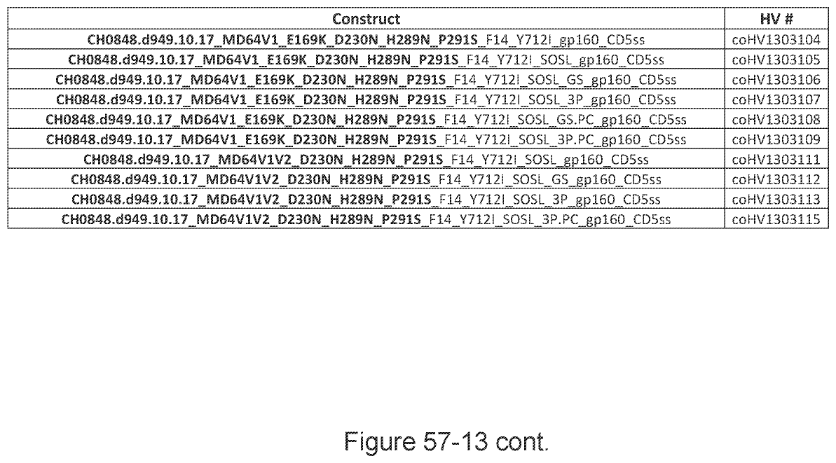

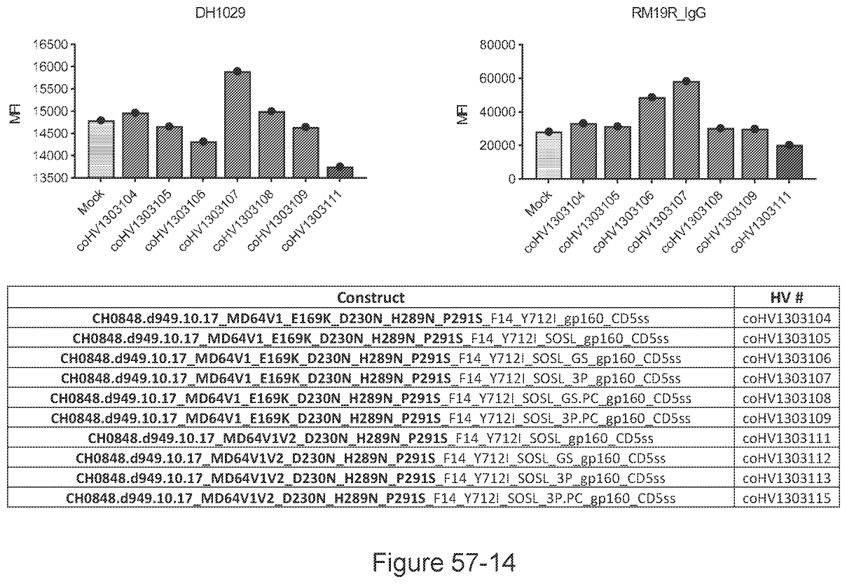

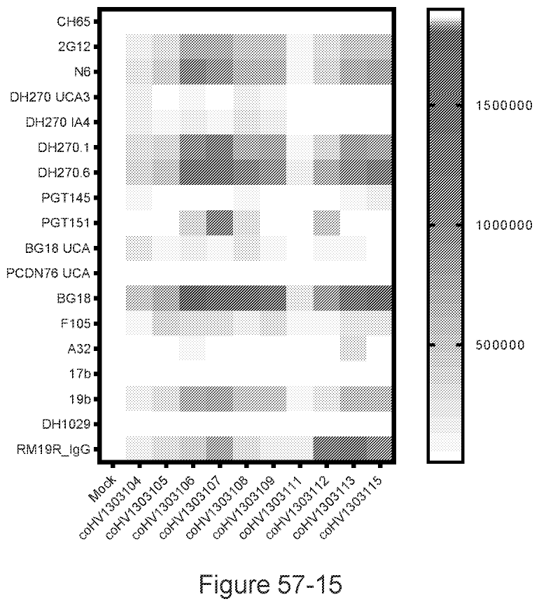

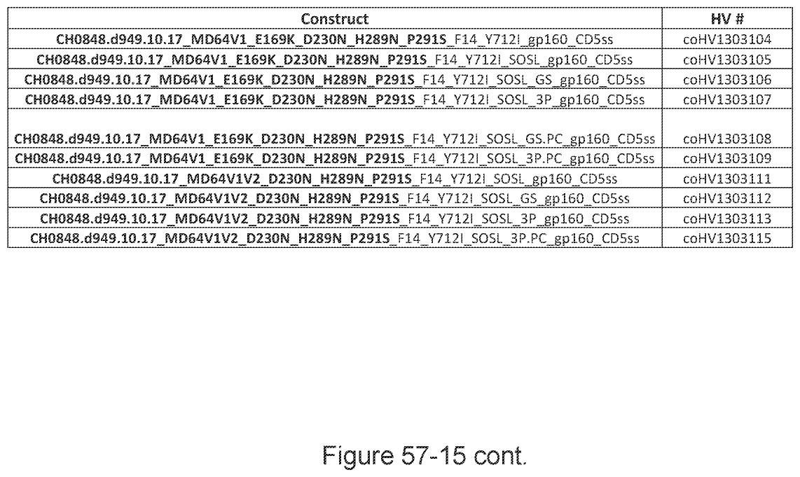

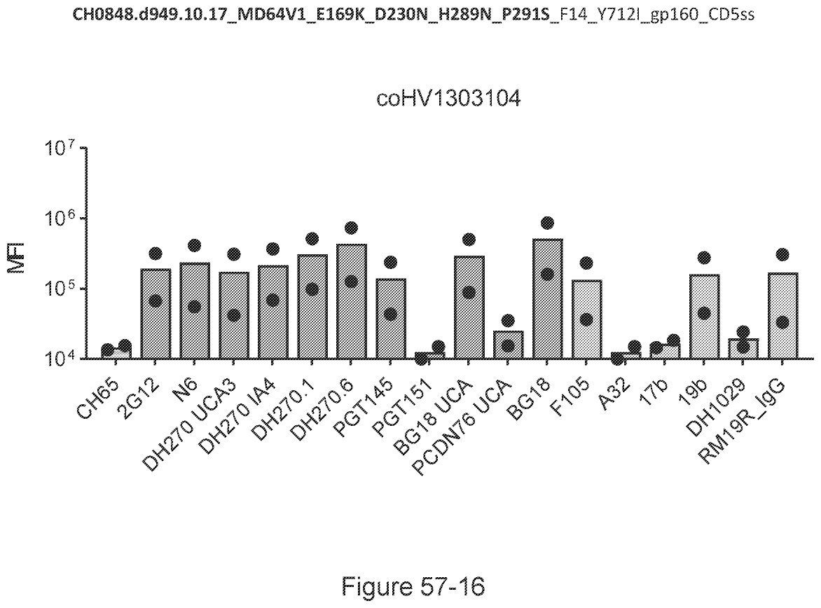

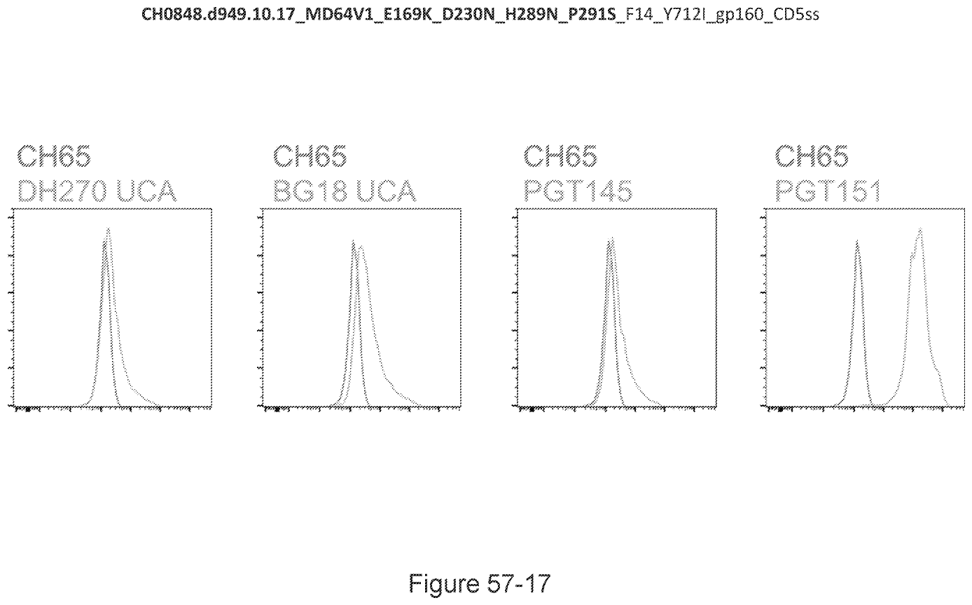

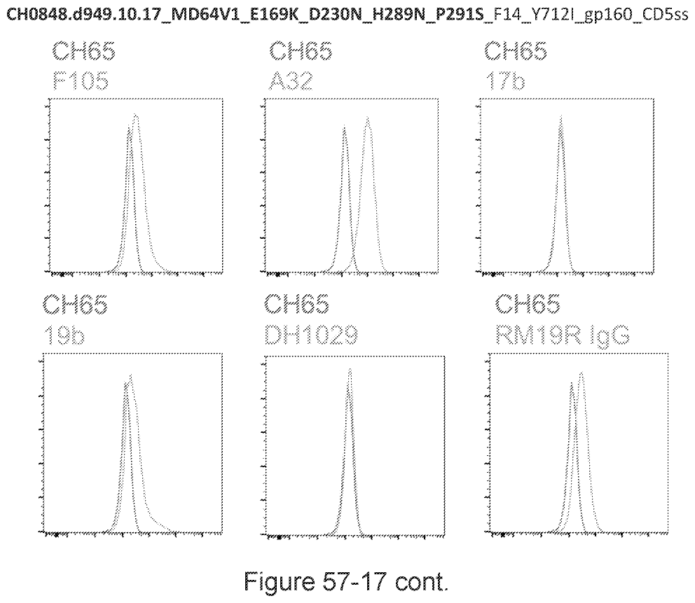

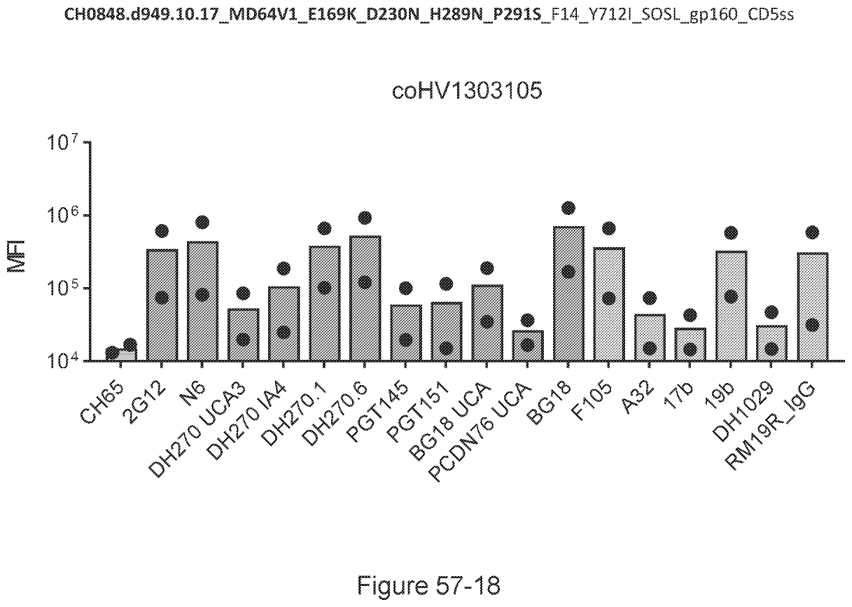

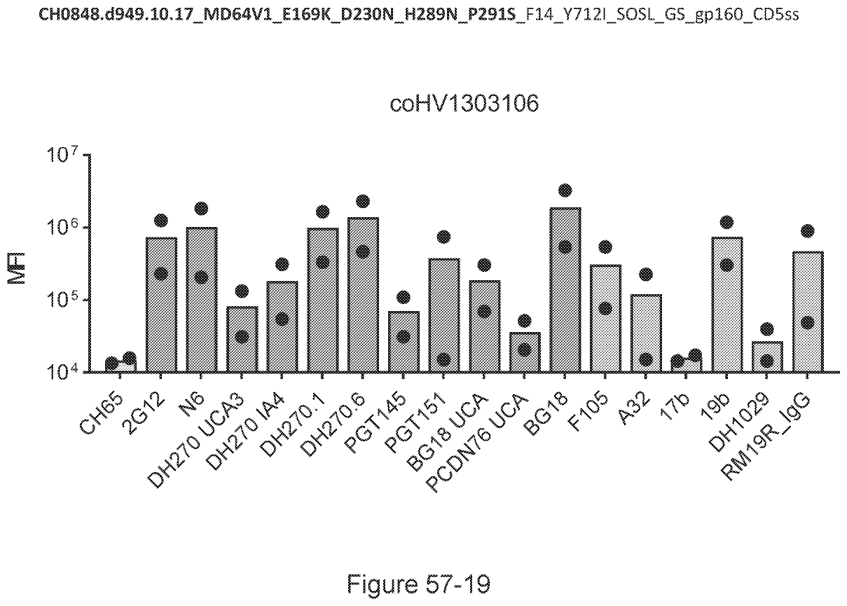

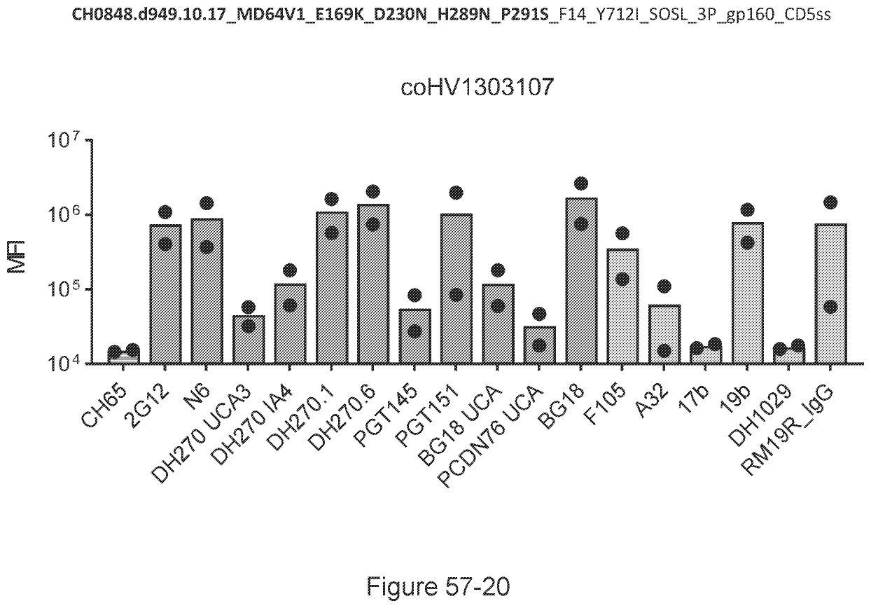

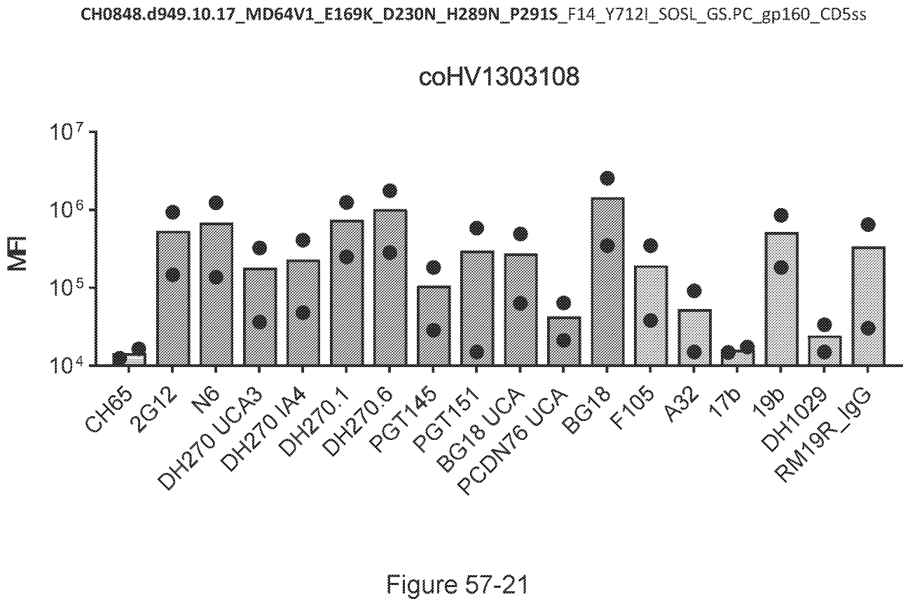

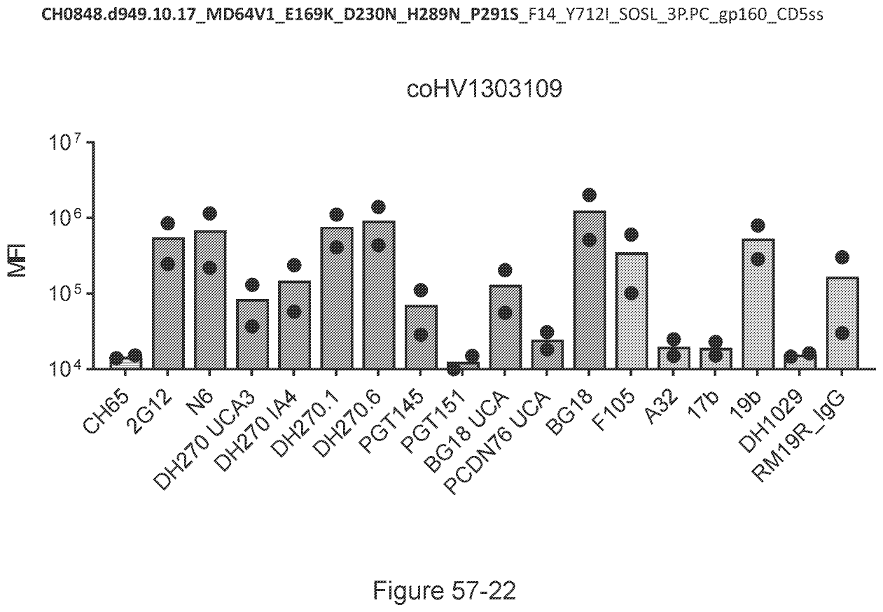

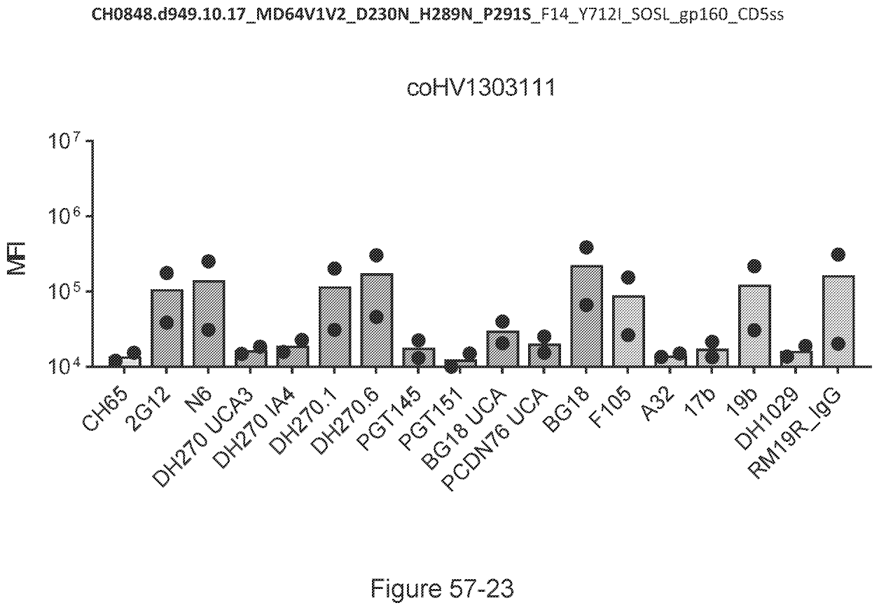

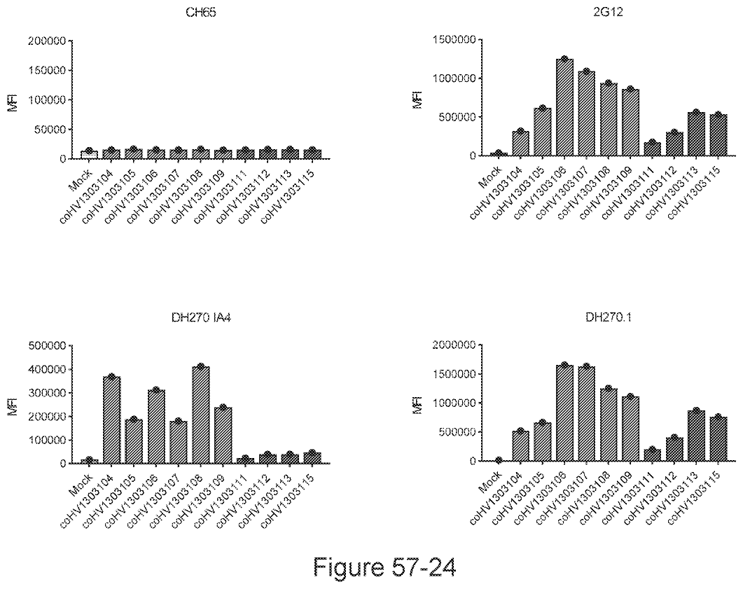

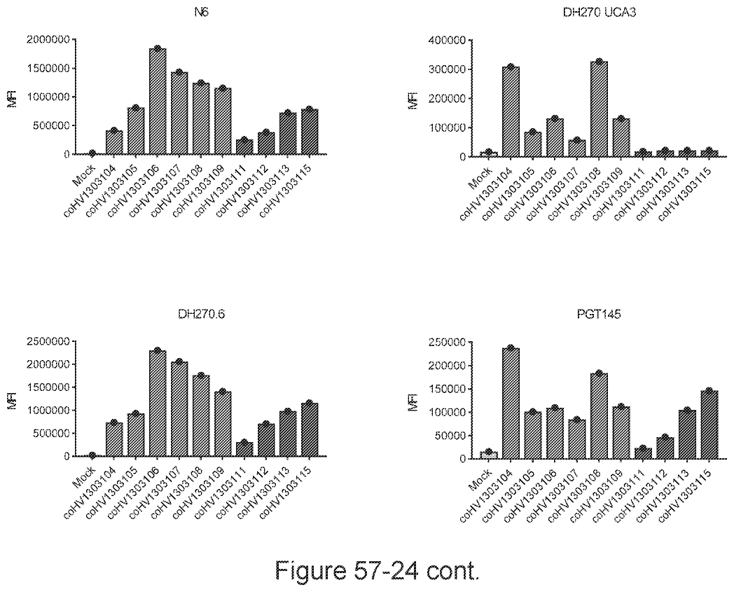

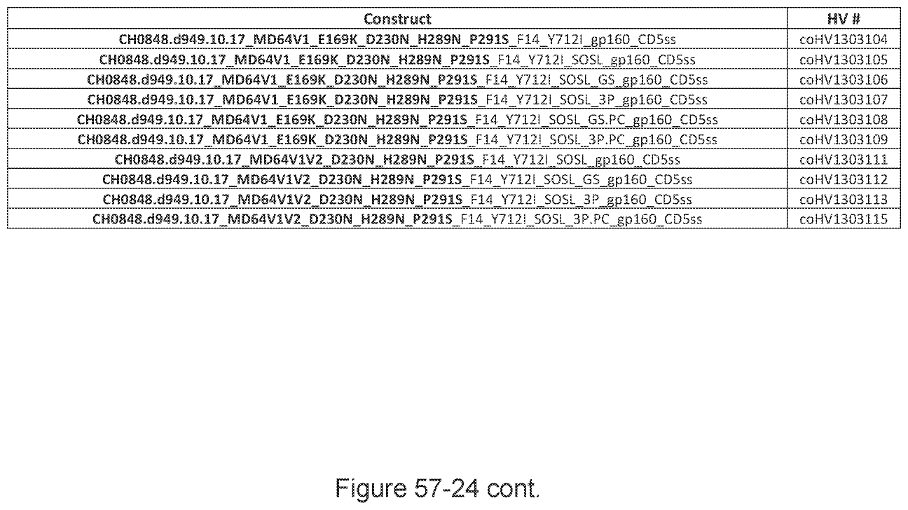

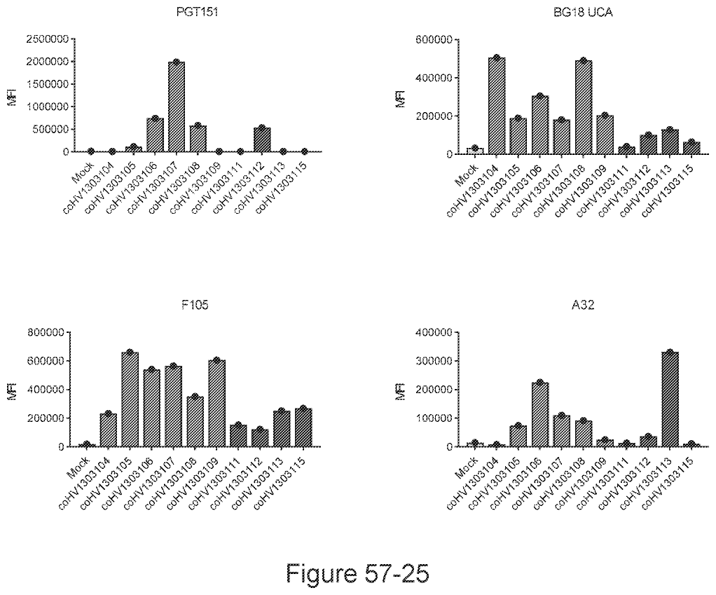

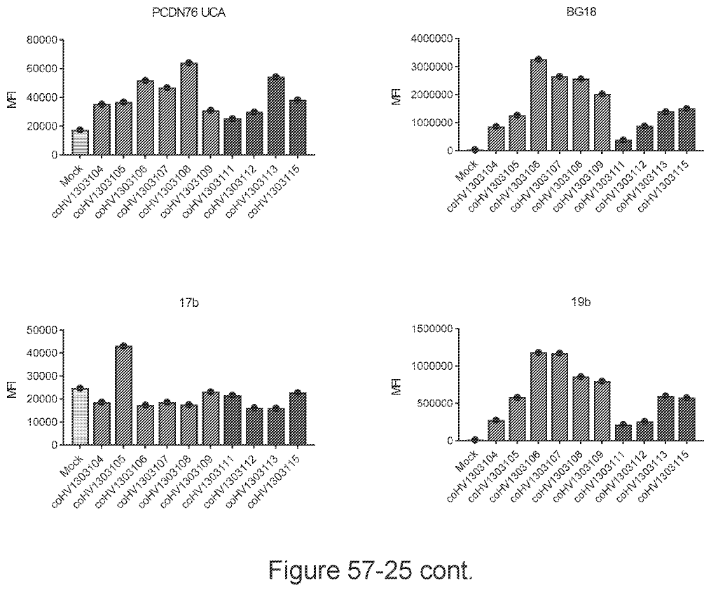

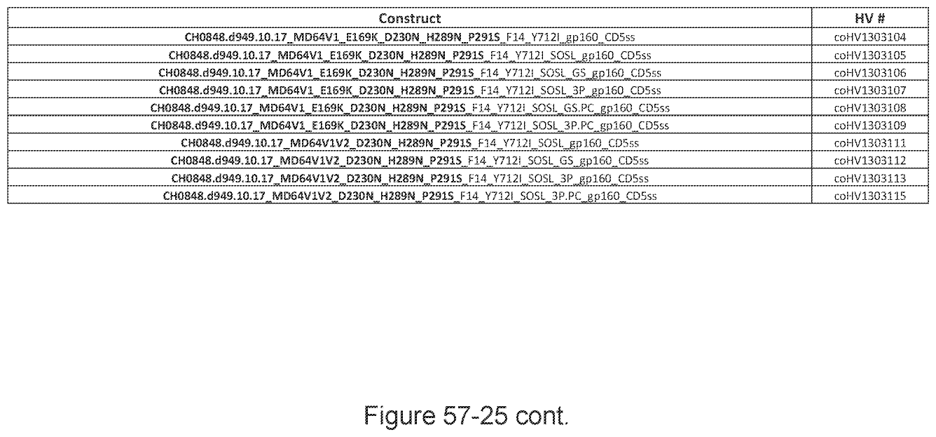

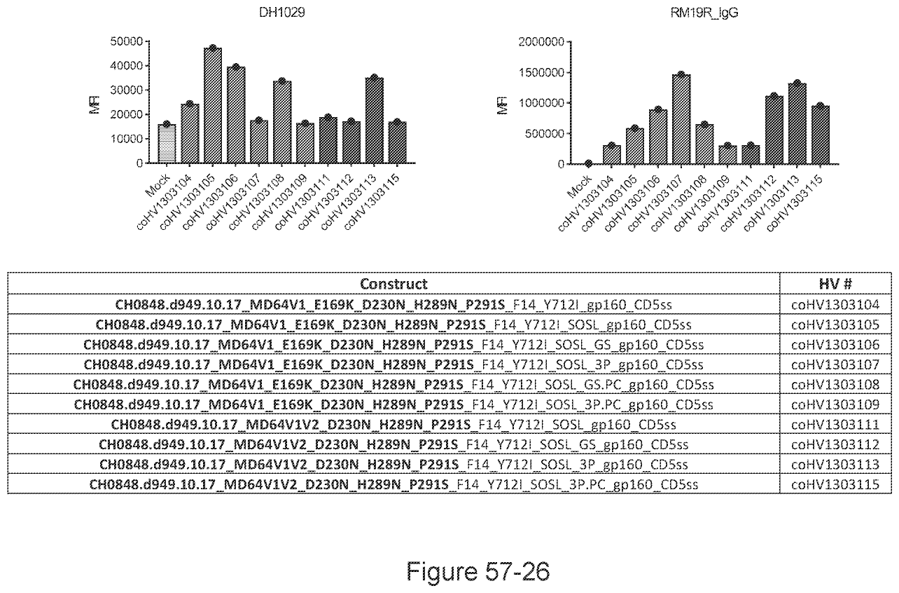

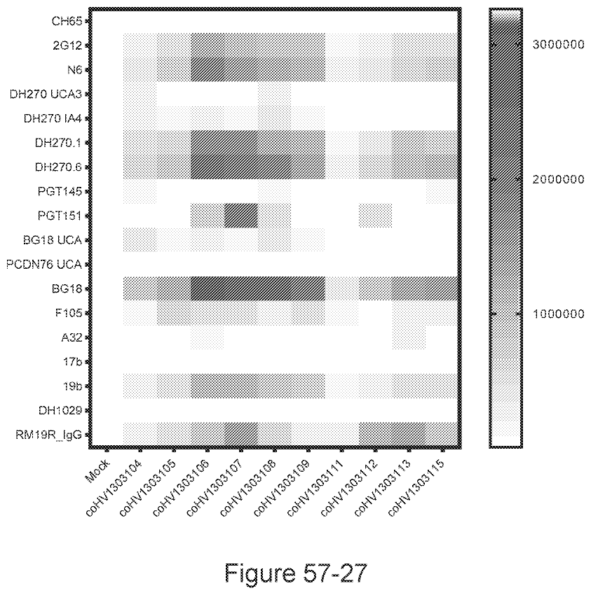

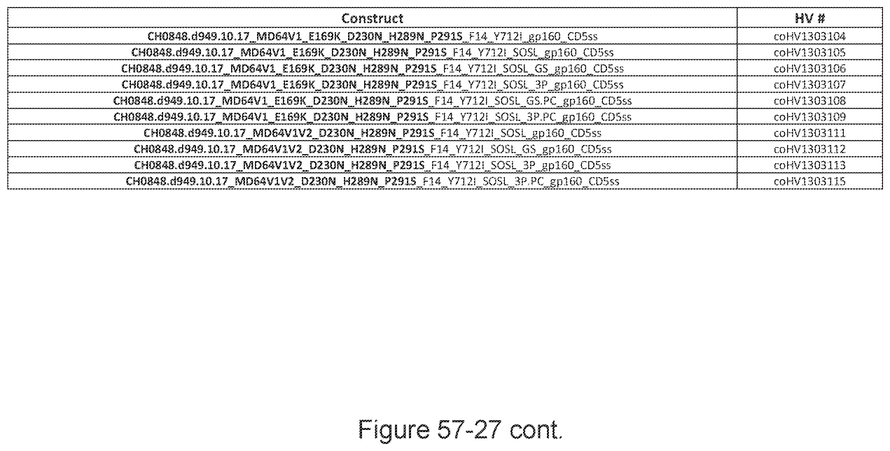

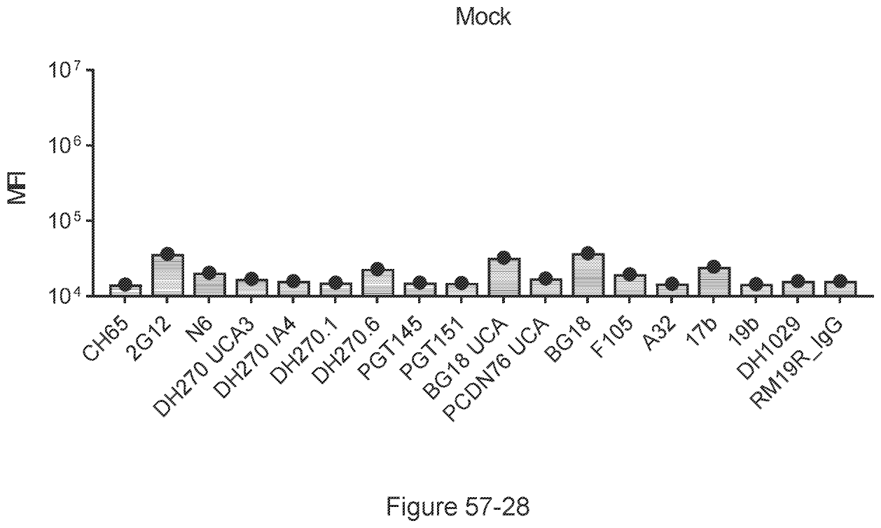

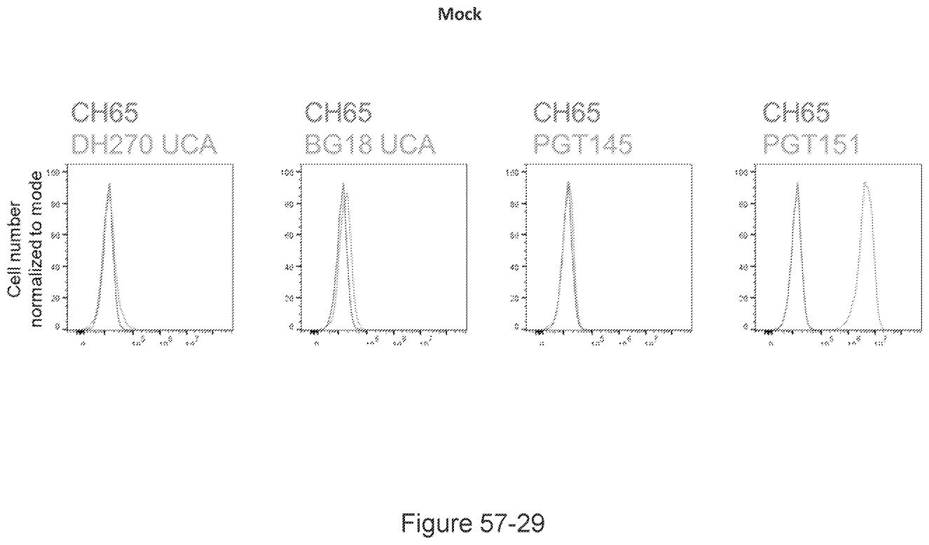

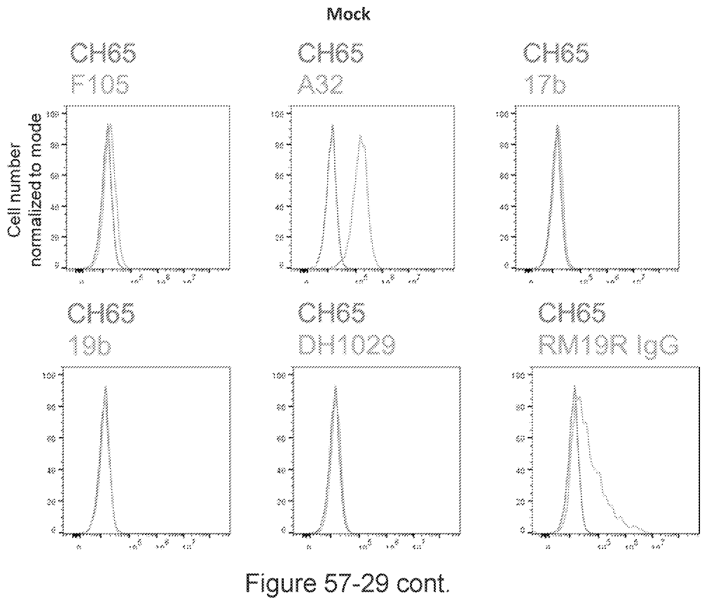

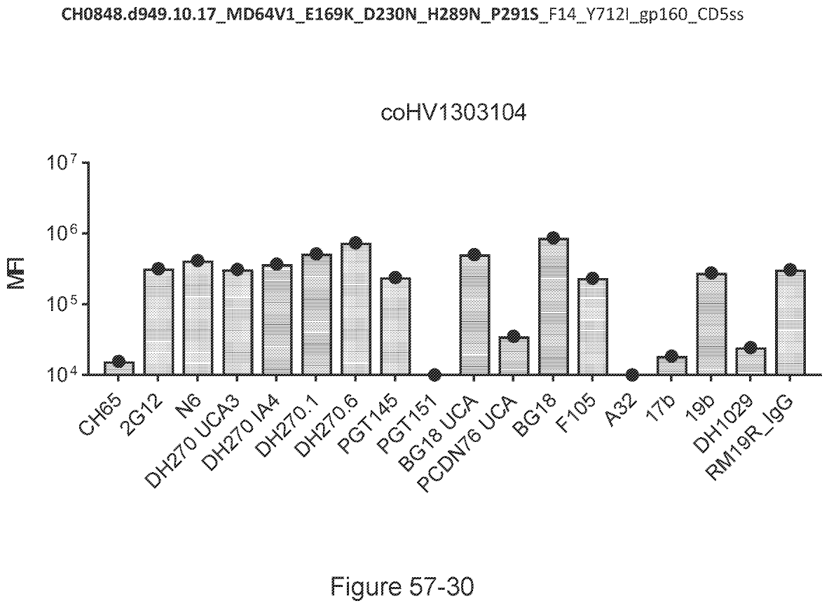

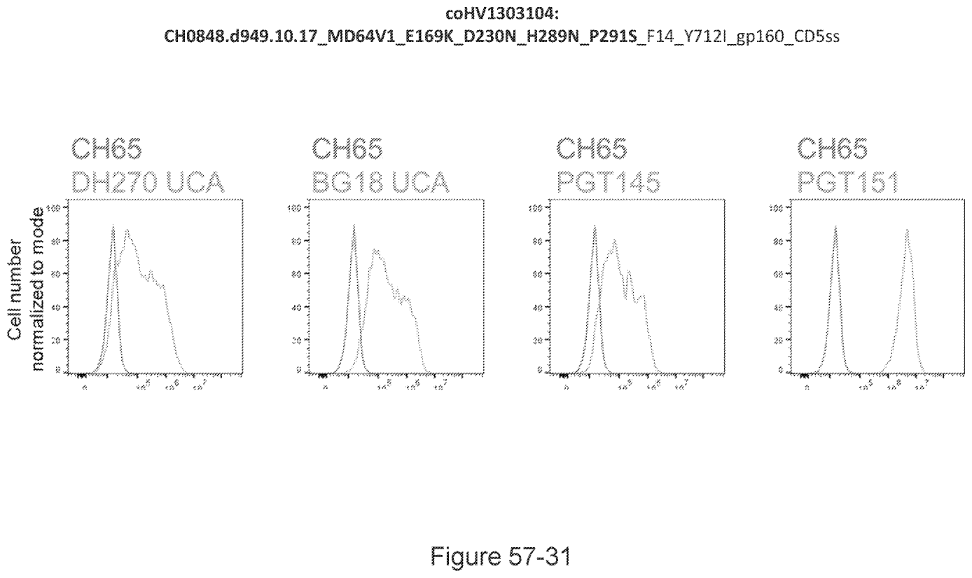

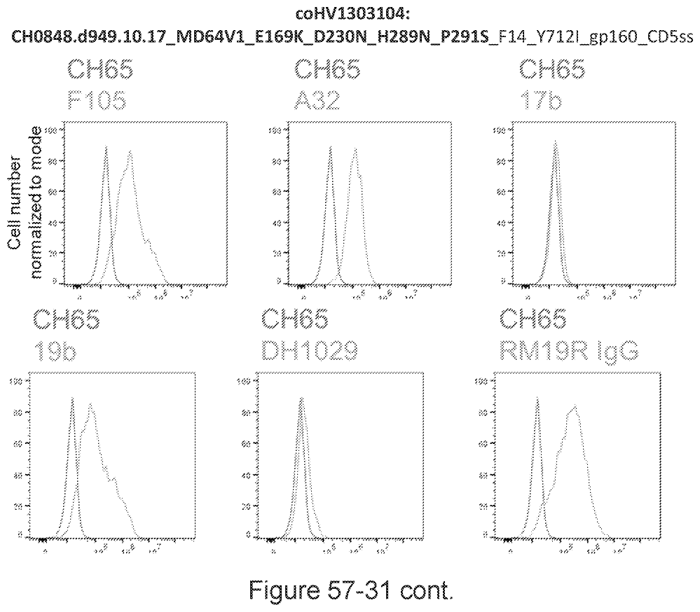

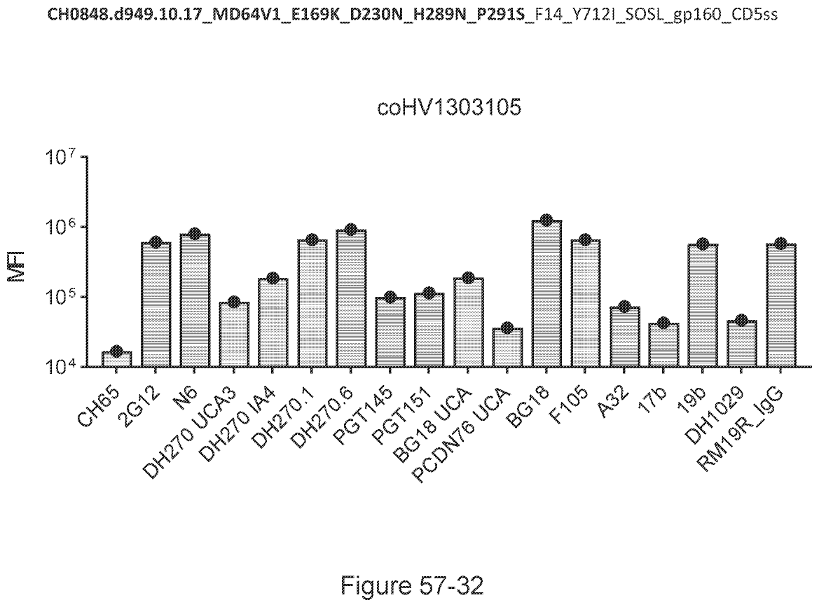

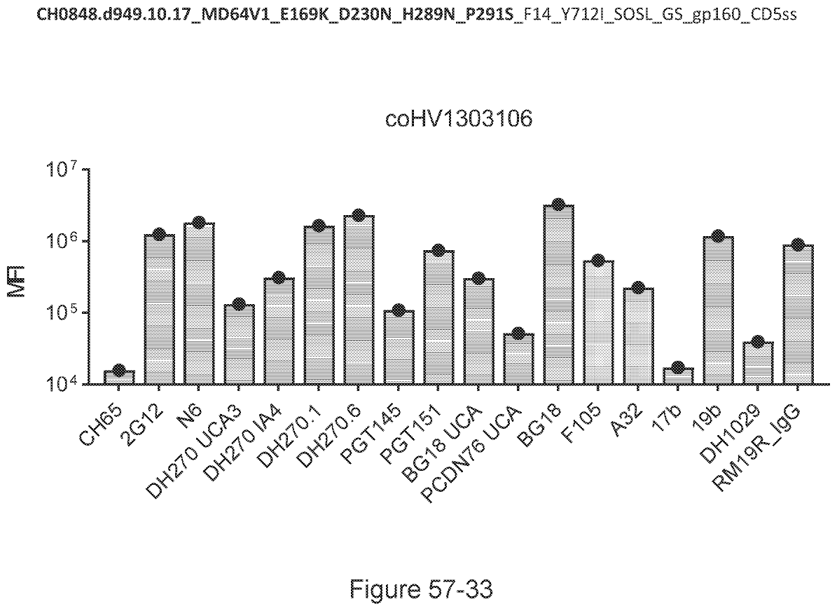

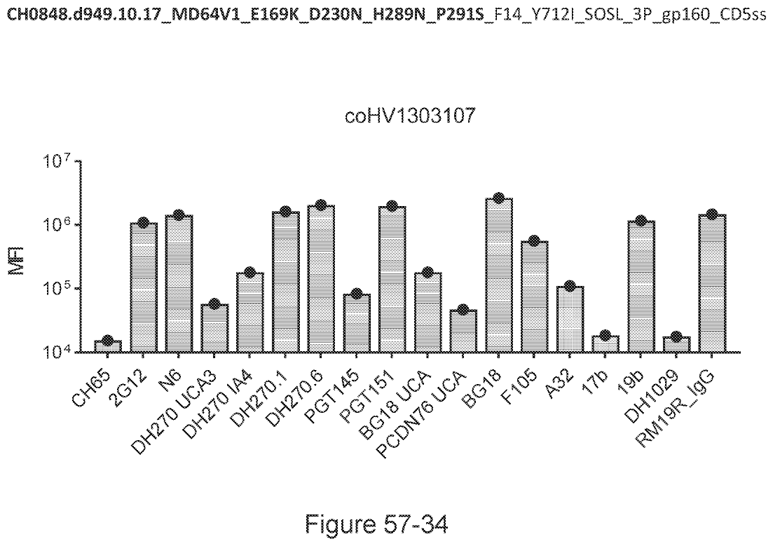

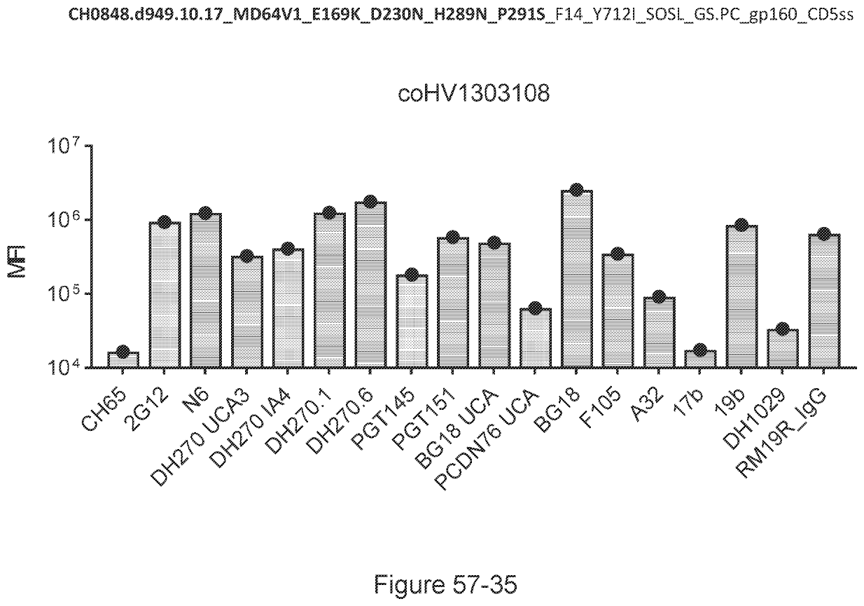

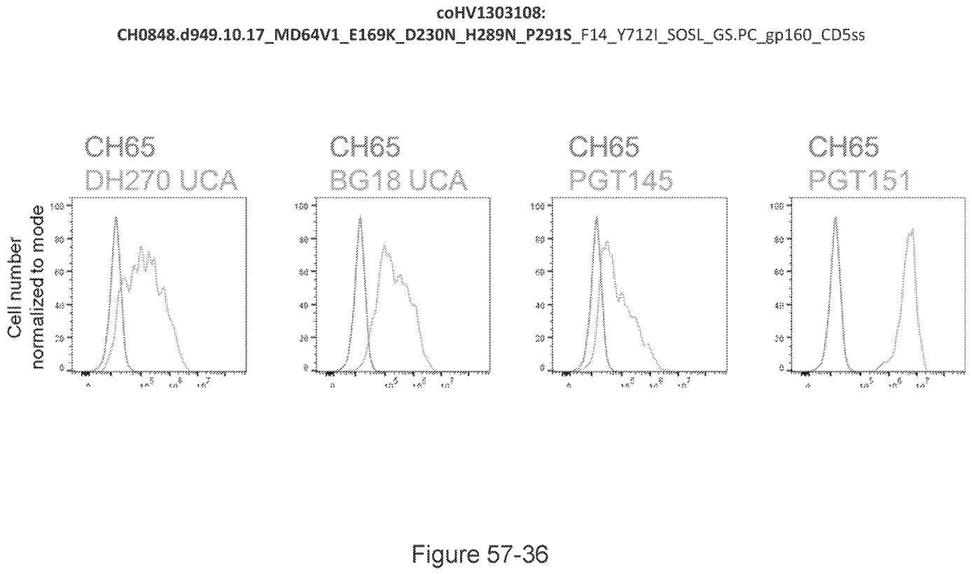

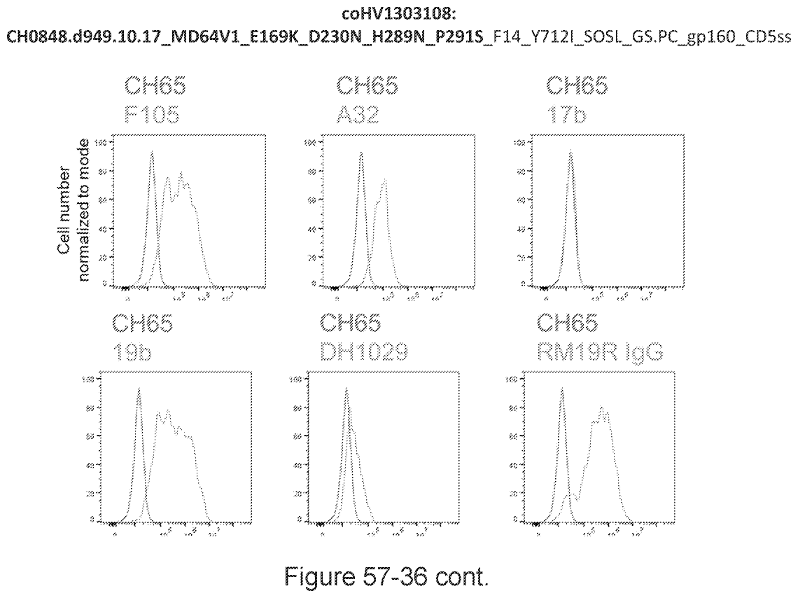

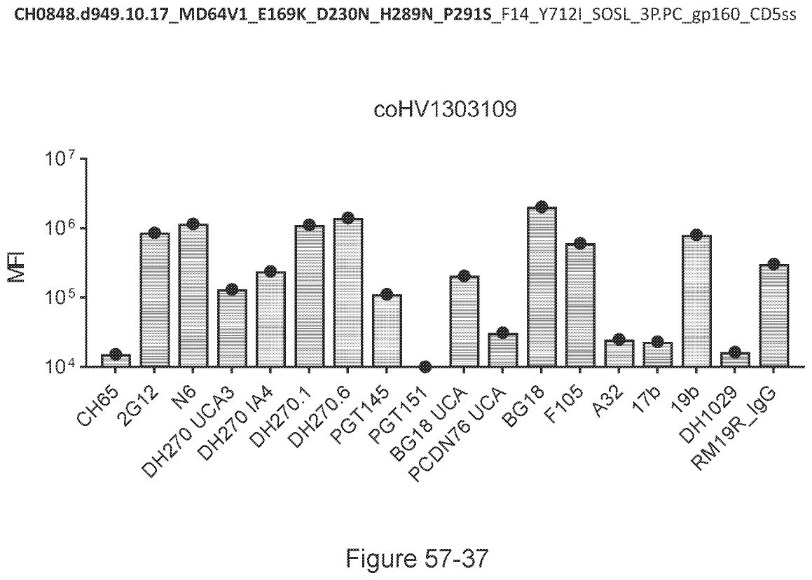

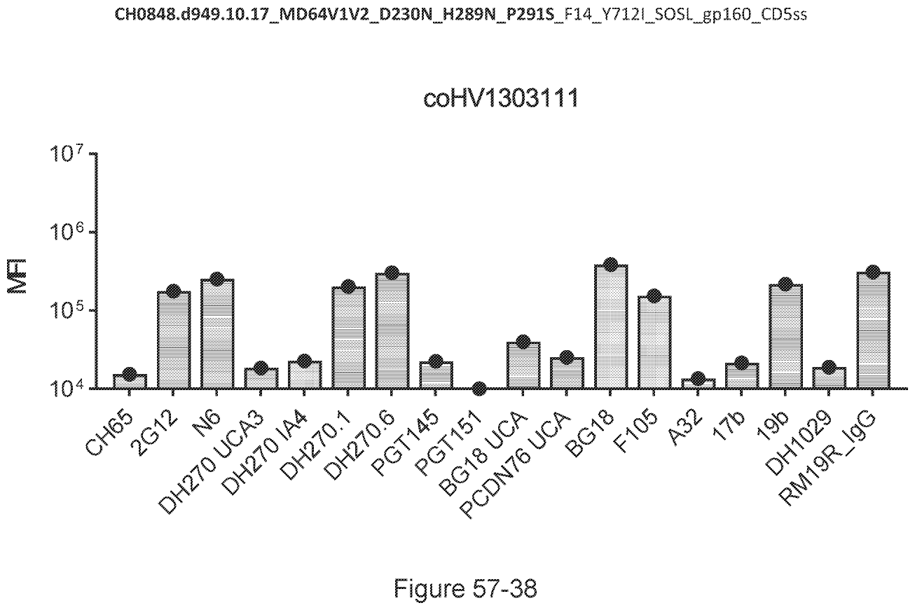

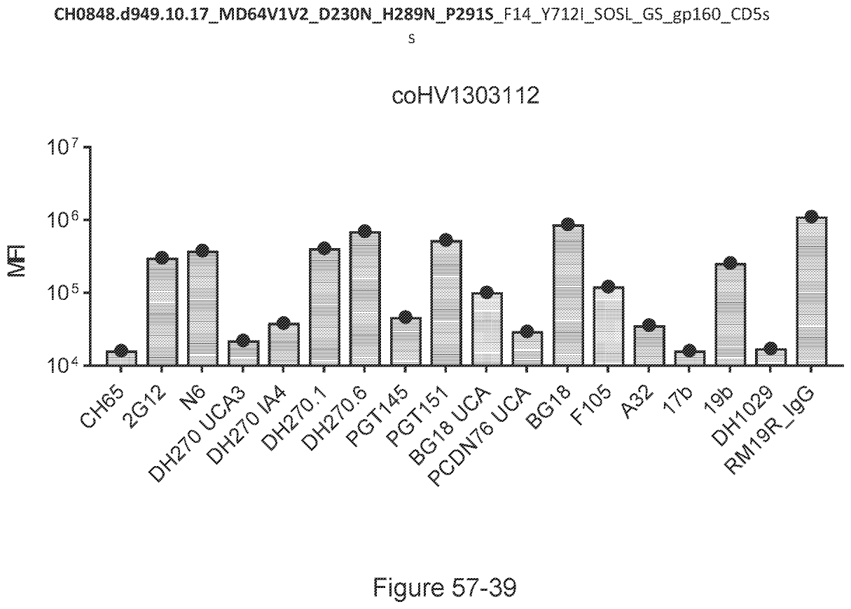

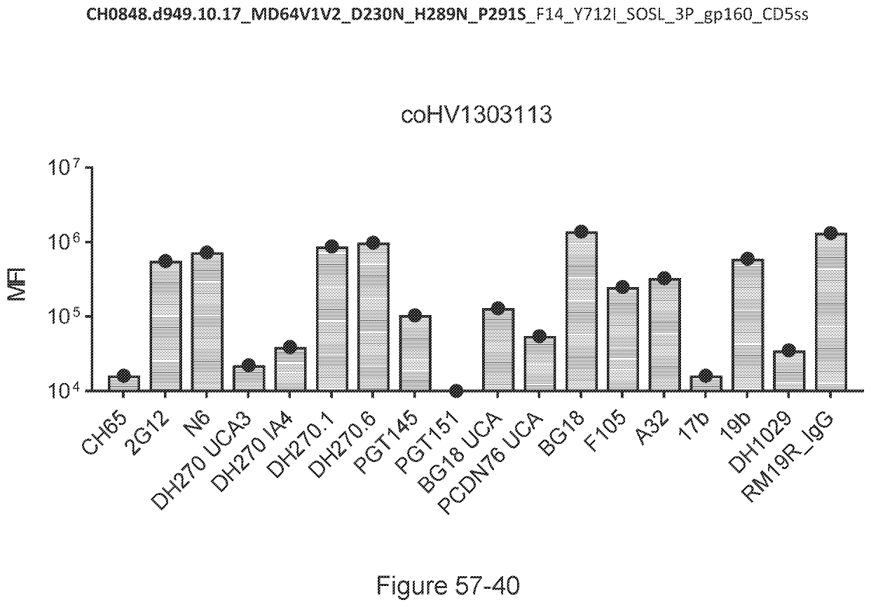

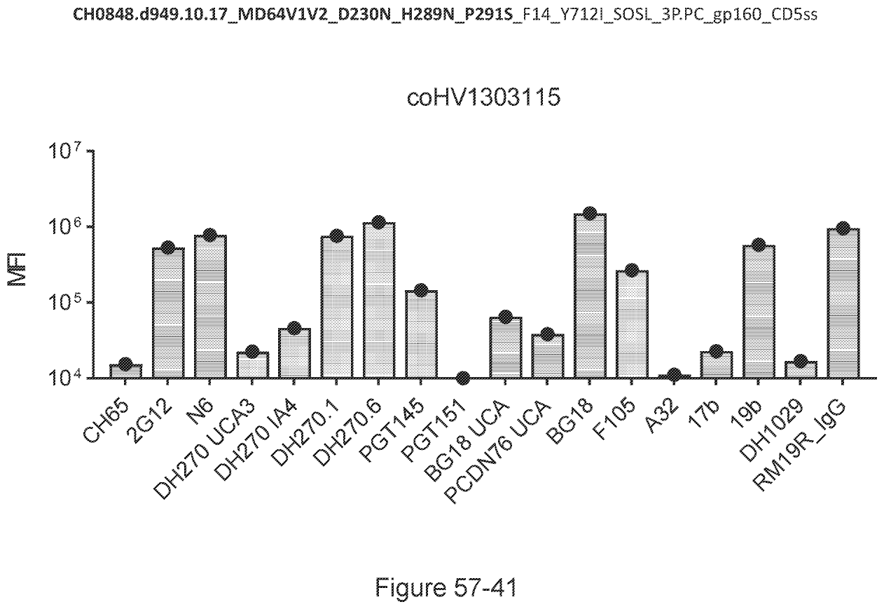

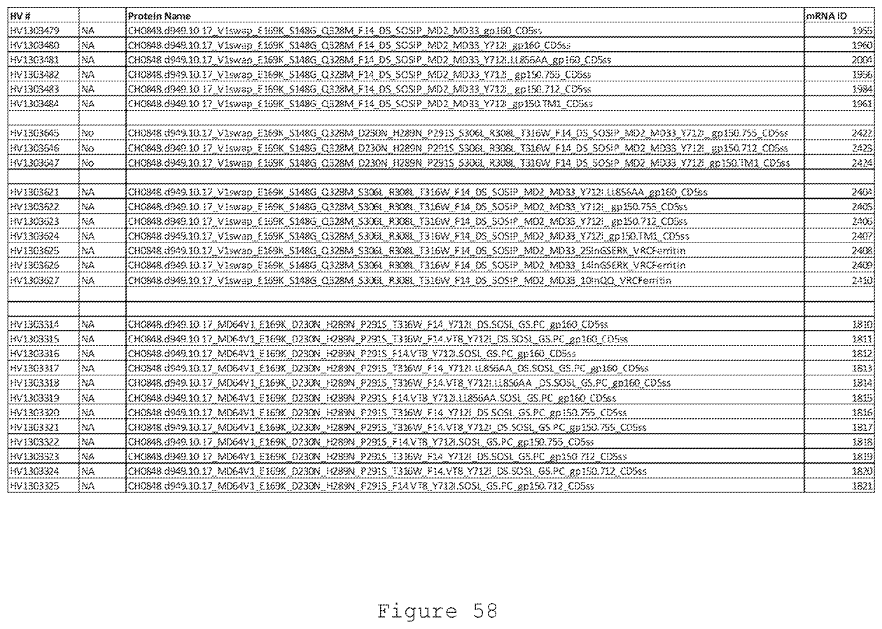

FIG. 57 parts 1-41 show CH848 MD64 gp160 down-selection summary. Based on data from three experiments, these two were down-selected:

HV1303104:CH0848.d949.10.17_MD64V1_E169K_D230N_H289N_P291S_F14_Y712I_g p160_CD5ss, and
HV1303108:CH0848.d949.10.17_MD64V1_E169K_D230N_H289N_P291S_F14_Y712I_S OSL_GS.PC_gp160_CDSss. CH848 10.17 MD64V1V2 constructs did not bind to DH270 UCA. In Trans #164, CH848 10.17DT F14 was also tested side-by-side with these constructs. CH848 10.17DT F14 had the highest DH270 UCA binding, but no BG18 UCA binding. It also showed minimal 19b binding, contrary to the CH848 10.17 MD64 constructs, to which 19b binding was somewhat high. Many Env constructs showed increased binding along the DH270 lineage, the trend held true in all three experiments. This hasn't been observed with CH848 10.17DT F14. FIGS. 57-1-57-2 show the three sets of binding reactions of Envs coHV1303104-coHV1303109, coHV1303111-coHV1303113, coHV1303115 and HV1301581_F14. FIGS. 57-3-57-4 show three sets of relative flow cytometric histogram of Env coHV1303104 and coHV1303108.

FIGS. 57-5-57-7 show the binding reaction of Envs coHV1303104-coHV1303109, coHV1303111-coHV1303113, coHV1303115 and HV1301581_F14. FIGS. 57-8-57-11 show relative flow cytometric histogram of Envs coHV1303104, coHV1303108, coHV1303106.

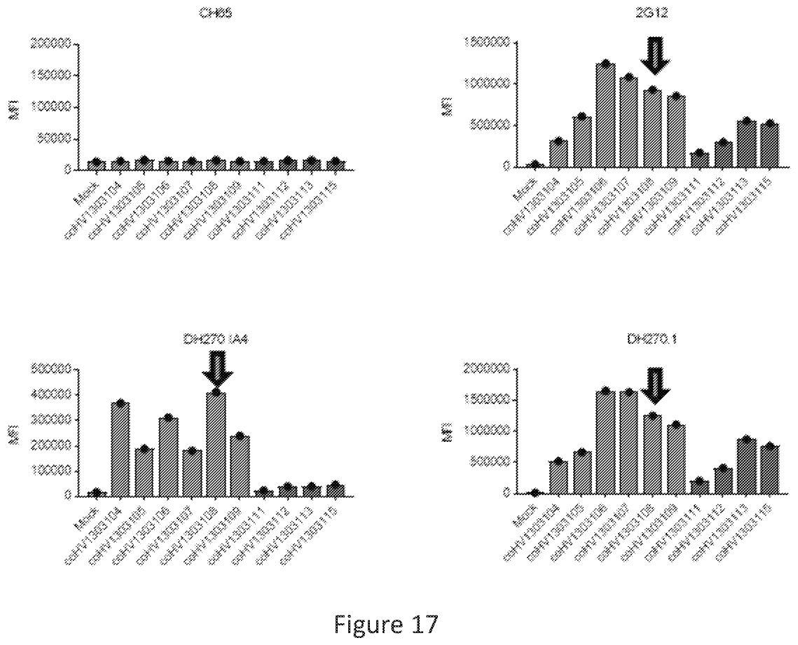

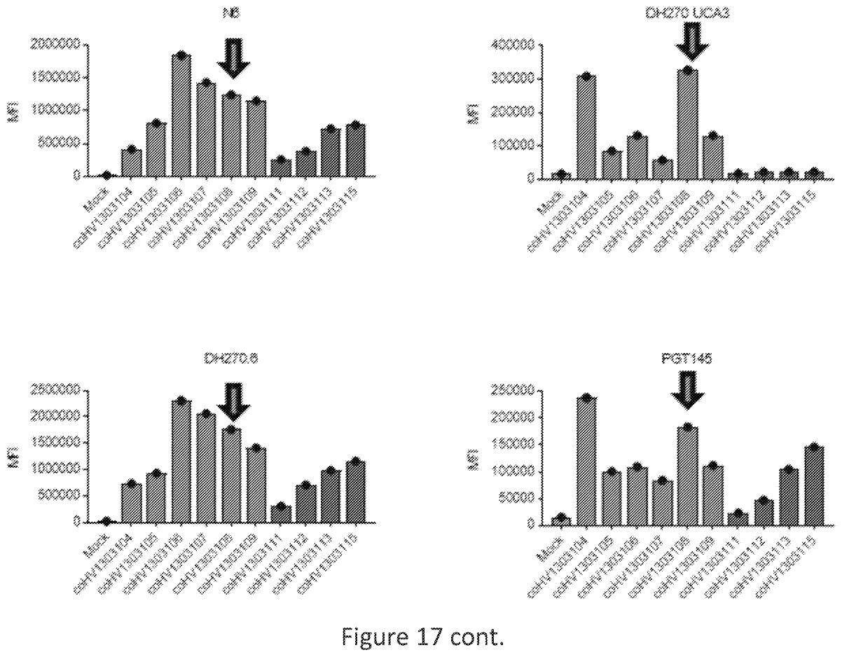

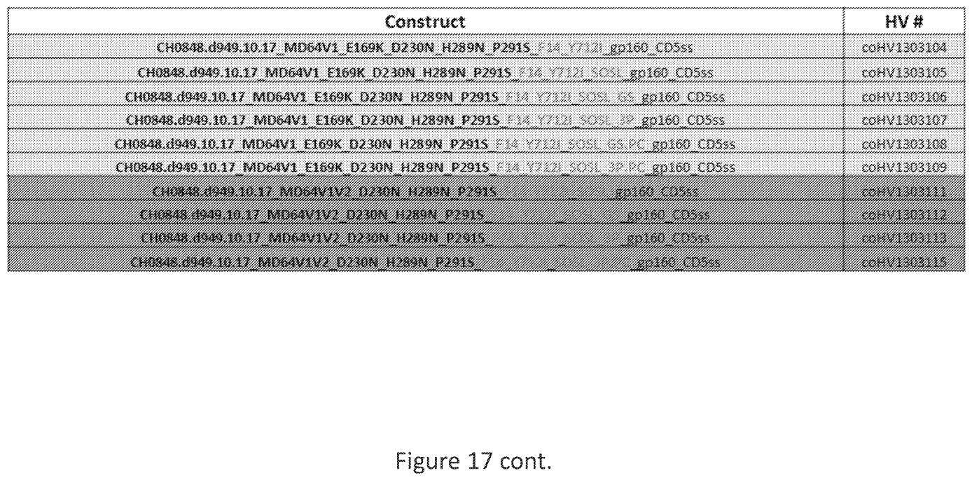

FIGS. 57-12-57-14 show the binding reaction of Enva coHV1303104-coHV1303109, coHV1303111-coHV1303113 and coHV1303115. FIG. 57-15 shows the heatmap of MFI of all antibodies binding to Envs coHV1303104-coHV1303109, coHV1303111-coHV1303113 and coHV1303115. FIG. 57-16 shows the binding reaction of Env coHV1303104. FIGS. 56-17 shows relative flow cytometric histogram of CH0848.d949.10.17_MD64V1_E169K_D230N_H289N_P291S_F14_Y712I_gp160_CD5ss. PGT151 binding on mock transfected cell was equally high. FIGS. 57-18-57-23 show the binding reaction of Envs coHV1303105-coHV1303109 and coHV1303111.

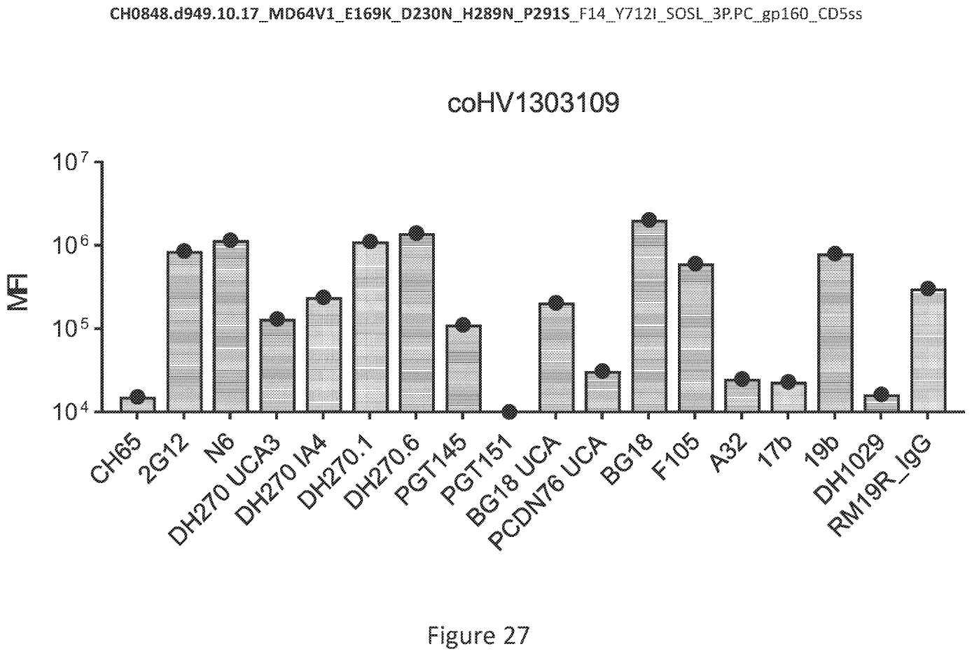

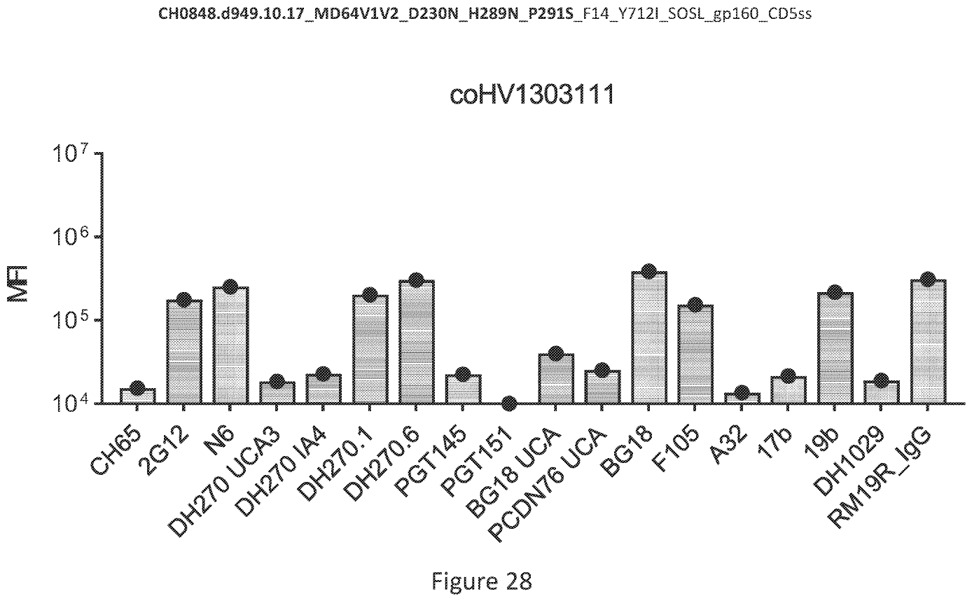

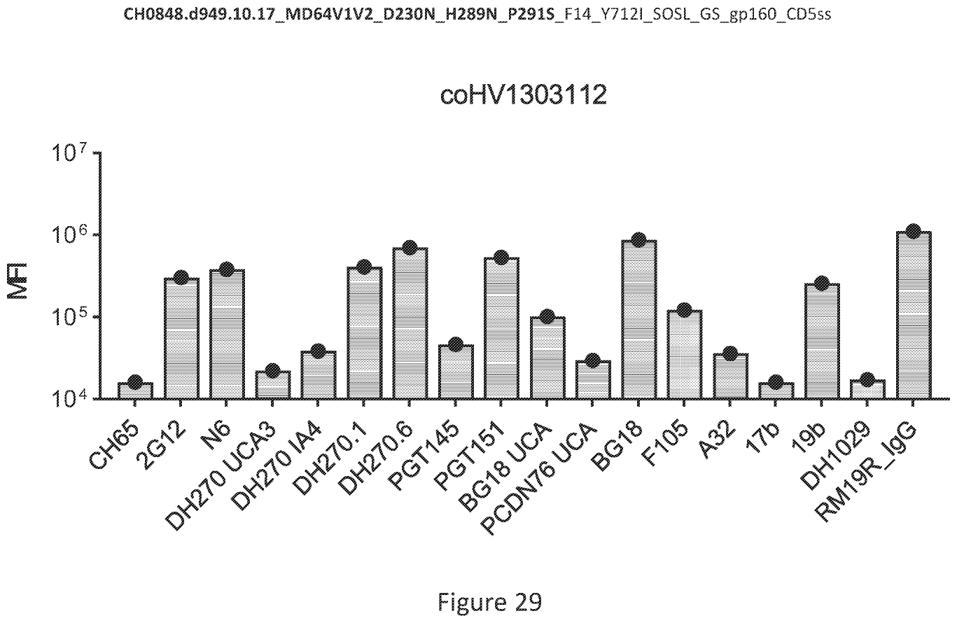

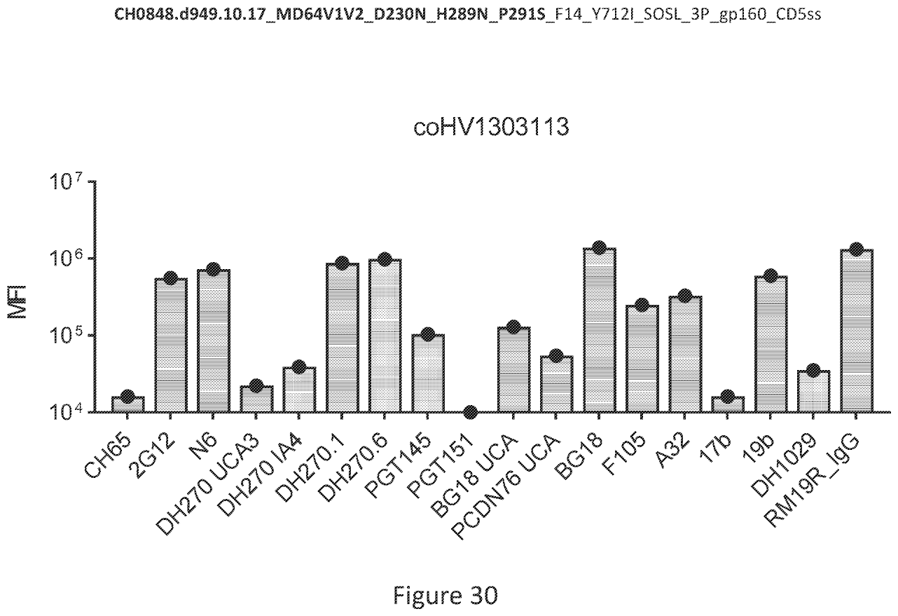

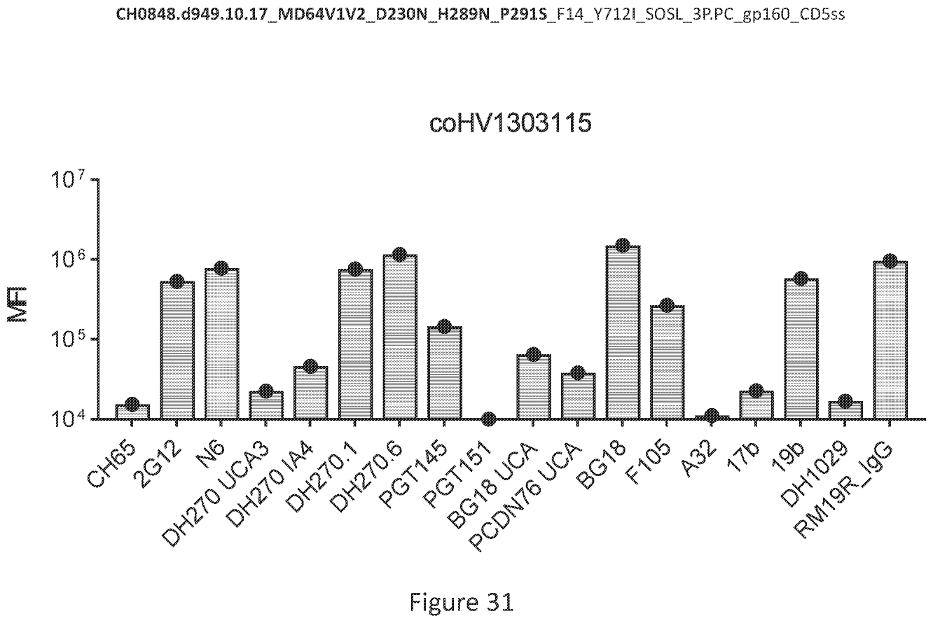

FIGS. 57-24-57-26 show the binding reactions of Envs coHV1303104-coHV1303109, coHV1303111-coHV1303113 and coHV1303115. FIG. 57-27 shows the heatmap of MFI of all antibodies binding to Envs coHV1303104-coHV1303109, coHV1303111-coHV1303113 and coHV1303115. FIG. 57-28 shows the binding reaction of the mock sample. FIG. 57-29 shows relative flow cytometric histogram of the mock sample. FIG. 57-30 shows the binding experiment of Env coHV1303104. FIG. 57-31 shows elative flow cytometric histogram of Env coHIV1303104. FIGS. 57-32-57-35 shows the binding reactions of Envs coHV1303105-coHV1303108. FIG. 57-36 shows relative flow cytometric histogram of Env coHV1303108. FIGS. 57-37-57-41 show the binding reaction of Envs coHV1303109, coHV1303111-coHV1303113 and coHV1303115.

What is claimed is:

1. A recombinant HIV-1 envelope comprising all the consecutive amino acids after the signal peptide of SEQ ID NOs: 19-30, 33, 35-45, 52-63 or 288-303.

2. The recombinant HIV-1 envelope of claim 1, comprising all the consecutive amino acids after the signal peptide of SEQ ID NO: 19, 23, 290 or 291.

3. A composition comprising any one of the HIV-1 envelopes of claim 1 and a carrier.

4. The composition of claim 3, wherein the HIV-1 envelope is a protomer comprised in a trimer.

5. The composition of claim 4, wherein the HIV-1 envelope is comprised in a stable trimer.

6. A composition comprising a nanoparticle and a carrier, wherein the nanoparticle comprises any one of the HIV-1 envelopes of claim 1.

7. The composition of claim 6, wherein the nanoparticle is a ferritin self-assembling nanoparticle.

8. A composition comprising a nanoparticle and a carrier, wherein the nanoparticle comprises any one of the trimers of claim 4 or 5.

9. The composition of claim 8, wherein the nanoparticle is a ferritin self-assembling nanoparticle.

10. The composition of claim 9, wherein the nanoparticle comprises multimers of trimers.

11. The composition of claim 9, wherein the nanoparticle comprises 1-8 trimers.

12. A method of inducing an immune response in a subject comprising administering an immunogenic composition comprising any one of the recombinant HIV-1 envelopes the preceding claims or compositions of the preceding claims, in an amount sufficient to induce an immune response.

13. The method of claim 12, wherein the immunogenic composition is administered as a prime.

14. The method of claim 12, wherein the immunogenic composition is administered as a boost.

15. A nucleic acid encoding any of the HIV-1 envelopes of claim 1.

16. A nucleic acid sequence comprising SEQ ID NOs 64-94 or 304-330.

17. The nucleic acid of claim 15 or 16, wherein the nucleic acid is a mRNA.

18. The nucleic acid of claim 17, wherein the mRNA is encapsulated in a lipid nanoparticle.

19. A composition comprising the nucleic acid of claims 15-18 and a carrier.

20. The composition of claim 3-11 or 19 further comprising an adjuvant.

21. The nucleic acid of claims 15-18 or the composition of claim 19 wherein the nucleic acid is operably linked to a promoter, and optionally wherein the nucleic acid is inserted in an expression vector.

22. A composition comprising a nanoparticle and a carrier, wherein the nanoparticle comprises any one of the nucleic acids of claim 15-18 or 21.

23. The composition of claim 22, wherein the nucleic acid is a mRNA.

24. The composition of claim 22 or 23, wherein the nanoparticle is a lipid nanoparticle.

25. A method of inducing an immune response in a subject comprising administering an immunogenic composition comprising the nucleic acid of claim 15-18 or 21 or the composition of claim 19-21 in an amount sufficient to induce an immune response.

26. The method of claim 12-14 or 25, further comprising administering an agent which modulates host immune tolerance.

27. The method of any of claim 25 or 26, wherein the nucleic acid administered is a mRNA.

28. The method of any of claims 25-27, wherein the nucleic acid is encapsulated in a lipid nanoparticle.

29. The method of any of claim 12-14 or 25-28, further comprising administering one or more additional HIV-1 immunogens to induce a T cell response.

30. The method of claim 25, wherein the immunogenic composition is administered as a prime.

31. The method of claim 25, wherein the immunogenic composition is administered as a boost.

32. A method of inducing an immune response comprising administering an immunogenic composition comprising a prime immunogen comprising all the consecutive amino acids after the signal peptide of SEQ ID NOs: 19-30, 33, 35-45, 52-63 or 288-303 or a nucleic acid sequence encoding all the consecutive amino acids after the signal peptide of SEQ ID NOs: 19-30, 33, 35-45, 52-63 or 288-303 followed by at least one boost immunogen from Table 3 or Table 4, wherein the boost immunogens are administered in the order appearing in Table 4, in an amount sufficient to induce an immune response.

33. The method of claim 32, wherein the prime or boost immunogen are administered as a nanoparticle.

34. The method of claim 32, wherein the nanoparticle is a ferritin self-assembling nanoparticle.

35. The method of claim 32, wherein the prime or boost immunogen are administered as a nucleic acid mRNA-LNP formulation.

36. An immunogenic composition or composition of any of the preceding claims, wherein the composition comprises at least two different recombinant HIV-1 envelopes or nucleic acids encoding a HIV-1 envelope, or a combination thereof.

37. An immunogenic composition comprising a first immunogen and a second immunogen, wherein the first immunogen is a HIV-1 envelope comprising all the consecutive amino acids after the signal peptide of SEQ ID NOs: 19-30, 33, 35-45, 52-63 or 288-303, or a nucleic acid sequence comprising SEQ ID NOs: 64-94 or 304-330, and wherein the second immunogen is a different HIV-1 envelope comprising all the consecutive amino acids after the signal peptide of SEQ ID NOs: 19-30, 33, 35-45, 52-63 or 288-303, or a nucleic acid sequence comprising SEQ ID NOs: 64-94 or 304-330.

38. The immunogenic composition of claim 37, wherein at least one of the first immunogen and the second immunogen is a recombinant HIV-1 envelope protein sequence.

39. The immunogenic composition of claim 38, wherein the first immunogen and the second immunogen are both a recombinant HIV-1 envelope protein sequence.

40. The immunogenic composition of claim 37, wherein at least one of the first immunogen and the second immunogen is a nucleic acid.

41. The immunogenic composition of claim 40, wherein the first immunogen and the second immunogen are both a nucleic acid.

42. The immunogenic composition of claim 40 or 41, wherein the nucleic acid is an mRNA.

43. The immunogenic composition of claim 42, wherein the mRNA is encapsulated in an LNP.

44. The immunogenic composition according to any one of claims 37-43, further comprising one or more additional immunogens, wherein the one or more additional immunogens is different to the first and second immunogens.

45. The immunogenic composition according to any one of claims 37-43, wherein the immunogenic composition comprises a carrier.

46. The immunogenic composition according to any one of claims 37-43, wherein the immunogenic composition further comprises an adjuvant.

47. A method of inducing an immune response in a subject comprising administering the immunogenic composition according to any one of claims 37-46 in an amount sufficient to induce an immune response.

48. The method of claim 47, further comprising administering an agent which modulates host immune tolerance.

49. A recombinant trimer comprising three identical protomers of an envelope comprising all the consecutive amino acids after the signal peptide of SEQ ID NOs: 19-30, 33, 35-45, 52-63 or 288-303.

50. The recombinant trimer of claim 49, wherein the envelope comprises all the consecutive amino acids after the signal peptide of SEQ ID NO: 19.

51. The recombinant trimer of claim 49, wherein the envelope comprises all the consecutive amino acids after the signal peptide of SEQ ID NO: 23.

52. The recombinant trimer of claim 49, wherein the envelope comprises all the consecutive amino acids after the signal peptide of SEQ ID NO: 290.

53. The recombinant trimer of claim 49, wherein the envelope comprises all the consecutive amino acids after the signal peptide of SEQ ID NO: 291.

54. An immunogenic composition comprising the recombinant trimer of any one of claims 49-53 and a carrier.

55. A composition comprising a nanoparticle and a carrier, wherein the nanoparticle comprises any one of the trimers of claims 49-53.

56. The composition of claim 55, wherein the nanoparticle is ferritin self-assembling nanoparticle.

57. The composition of claim 55, wherein the nanoparticle comprises multimers of trimers.

58. The composition of claim 55, wherein the nanoparticle comprises 1-8 trimers.
